SEQUENCE LISTING (716 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 577 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 4 - protein, 556 aa, Homo sapiens
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 5 - protein, 325 aa, Homo sapiens
MAWSLGSWLGGCLLVSALGMVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 6 - protein, 306 aa, Homo sapiens
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPSWMVAVILMASVFMVCLALLGCFALLWCVYKKTKYAFSPRNSLPQHLKEFLGHPHHNTLLFFSFPLSDENDVFDKLSVIAEDSESGKQNPGDSCSLGTPPGQGPQS

SEQ ID NO: 7 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 8 - protein, 157 aa, Homo sapiens [sequence omitted]

SEQ ID NO: 9 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 10 - protein, 171 aa, Homo sapiens
LPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 11 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 12 - protein, 158 aa, Homo sapiens
LIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 13 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 14 - protein, 152 aa, Homo sapiens
QIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 15 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 16 - protein, 180 aa, Homo sapiens
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 17 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 18 - protein, 426 aa, Homo sapiens
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 19 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 20 - protein, 439 aa, Homo sapiens
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 21 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 22 - protein, 245 aa, Homo sapiens
LPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 23 - protein, 335 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 24 - protein, 310 aa, Homo sapiens
QVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 25 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 26 - protein, 393 aa, Homo sapiens
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 27 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 28 - protein, 271 aa, Homo sapiens
VAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 29 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 30 - protein, 405 aa, Homo sapiens
QAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 31 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 32 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 33 - protein, 577 aa, Homo sapiens
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 34 - protein, 180 aa, synthetic construct
MGKRAFVVSVAMALLGIYVITNTVNARHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 35 - protein, 197 aa, synthetic construct
MARRLTVASCGSVSLLAAFAAVLLIGCQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 36 - protein, 186 aa, synthetic construct
MSNNKILVCAVIILTYTLYTDAYCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 37 - protein, 199 aa, synthetic construct
MANVVYVVLVISIMMANIHVSKTYCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 38 - protein, 182 aa, synthetic construct
MQGLQLLRGLLCCGVFAAASSRSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 39 - protein, 187 aa, synthetic construct
MRRRRSFGIVVSGAIRTLLMVAVVAVSVRGHEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 40 - protein, 185 aa, synthetic construct
MGSRRLSRCSFATAVCLVAIVAAVAAKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 41 - protein, 175 aa, synthetic construct
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 42 - protein, 182 aa, synthetic construct
MALAHQLPVWIFSIWILYFTLPLSEERVLPLRGNCKLLLQDTVIPNLLYSMRSIFQDIKPYFQGKDSLNNLLLSGQLLEDLQSPIGCDALSEMIQFYLEEVMPQAEIHHPKHKNSVMQLGETLHTLISQLQECTALFPCKHKSLGAQKIKEEVSKLGQYGIIKAVAEFDIFINYMESYFGVK

SEQ ID NO: 43 - protein, 189 aa, synthetic construct
MRRRRRSFGIIVAGAIGTLLMMAVVVLSAHDHEHKEVPPACDPVHGNLAGIFKELRATYASIREGLQKKDTVYYTSLFNDRVLHEMLSPMGCRVTNELMEHYLDGVLPRASHLDYDNSTLNGLHVFASSMQALYQHMLKCPALACTGKTPAWMYFLEVEHKLNPWRGTAKAAAEADLLLNYLETFLLQF

SEQ ID NO: 44 - protein, 186 aa, synthetic construct
MRRRRSFGIVVAGAIGTLLMMAVVVFSAHEHKEVPPACDPVHGNLAGIFKELRATYASIREGLQKKDTVYYTSLFNDRVLQEMLSPMGCRVTNELMEHYLDGVLPRALHLDYDNSTLNGLHAFASSMQALYQHMLKCPALACTGKTPAWMYFLEVEHKLNPWRGTAKAAAEADLLLNYLETFLLQF

SEQ ID NO: 45 - protein, 185 aa, synthetic construct
MSKNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKP

SEQ ID NO: 46 - protein, 185 aa, synthetic construct
MSNNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKT

SEQ ID NO: 47 - protein, 185 aa, synthetic construct
MPNNKILVCAVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKS

SEQ ID NO: 48 - protein, 184 aa, synthetic construct
MSNKKILVCVVIILTYTLYTDAYCVEYKESEEDRQQCSSSSFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTK

SEQ ID NO: 49 - protein, 185 aa, synthetic construct
MSNNKILVCVAIILTYTLYTDAYCVEYAESDEDKQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 50 - protein, 185 aa, synthetic construct
MSKNKVLVCFVIILTYTLYTDAYCVEYEESEEDKQQCGSNGGPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 51 - protein, 185 aa, synthetic construct
MSNNKILVCAVIILTYTLYTDAYCVEYAESDEDRQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 52 - protein, 186 aa, synthetic construct
MSNNKILLCVAIILTYTLYTDAYCVEYEESEEDKQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 53 - protein, 185 aa, synthetic construct
MSNNKILVCAVIILTYTLYTDAYCIQYEESEEDKQQCSSSNFPASLPHMLRELRAAFGKVKTFFQMKDQLNSMLLTQSLLDDFKGYLGCQALSEMIRFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 54 - protein, 185 aa, synthetic construct
MSKNKFLVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLREKLKTLRLRLRRCHRFLPCENKSKAVEQVKRVFNMLQERGVYKAMSEFDIFINYIESYMTTKT

SEQ ID NO: 55 - protein, 186 aa, synthetic construct
MSKNKILVCFVIILTYTLYTDAYCVEYEESEEDKQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 56 - protein, 185 aa, synthetic construct
MSNNKILVCVAIILTYTLYTDAYCVEYAESDEDKQQCSGSNFPASLPHMLRELRAAFGKVKTFFQMKDQLNSMLLTQSLLDDFKGYLGCQALSEMIQFYLEEVMPRAENHGPDIKEHVNSLGEKLKTLRLRLRRCHRFLPCENKSKAVEQVKRVFNMLQERGVYKAMSEFDIFINYIESYMTTKM

SEQ ID NO: 57 - protein, 186 aa, synthetic construct
MSNNKILVCVVIILTYTLYTDAYCVEYEESEEDRQQCSGSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 58 - protein, 186 aa, synthetic construct
MSNNKILVCAVIILTYTLYTDAYCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 59 - protein, 186 aa, synthetic construct
MSKNKILVCVAIILTYTLYTDAYCVEYEESDEDKQQCSSSTGAPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 60 - protein, 186 aa, synthetic construct
MSKNKILVCVAIILTYTLYTDAYCVEYEETKEDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 61 - protein, 185 aa, synthetic construct
MSKNKILVCVVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGDYKAMSEFDIFINYIESYMTTKS

SEQ ID NO: 62 - protein, 186 aa, synthetic construct
MSNNKILVCVVIILTYTLYTDAYCVEYEESEEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCEDKSKAVEQVKRVFNMLQERGVYKAMSEFDILINYIESYMTTKM

SEQ ID NO: 63 - protein, 186 aa, synthetic construct
MSNNKILVCAVIILTYTLYTDAYCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMATKM

SEQ ID NO: 64 - protein, 179 aa, synthetic construct
MFRALLLCCLALLAGVWADNRYDGQDGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 65 - protein, 186 aa, synthetic construct
MSKNKILVCVAIILTYTLYTDAYCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 66 - protein, 186 aa, synthetic construct
MSKNKILVCVAIILTYTLYTDAYCVEYEESKEDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 67 - protein, 186 aa, synthetic construct
MSKNKILVCVAIILTYTLYTDAYCVEYLESGEDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 68 - protein, 186 aa, synthetic construct
MSKNKILVCVAIILTYTLYTDAYCVEYLESREDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 69 - protein, 178 aa, synthetic construct
MPGAALLYCLFFVTGVWAESENNCTHFPTSLPHMLHELRAAFSRVKTFFQMKDQLDNMLLNGSLLEDFKGYLGCQALSEMIQFYLEEVMPQAENHSGGGGPDIKEHVNSLGEKLKTLRVRLRRCHRFLPCENKSKAVEQVKSAFSKLQEKGVYKAMSEFDIFINYIEAYMTTKMKNKK

SEQ ID NO: 70 - protein, 186 aa, synthetic construct
MSNNKILVCVAIILTYTLYTDAYCVEYLESDEDKQHCSSSNGASASSPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 71 - protein, 185 aa, synthetic construct
MSNNKILVCVAIILTYTLYTNAYCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLAEKLKTLRLRLRRCHRFLPCENKSKAVEQVKRVFNMLQERGVYKAMSEFDIFINYIESYMTTKS

SEQ ID NO: 72 - protein, 185 aa, synthetic construct
MSNNKILVCVAIILTYTLYTNAYCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLGEKLKTLRLRLRRCHRFLPCENKSKAVAQVKRVFNMLQERGVYKAMSEFDIFINYIESYMTTKS

SEQ ID NO: 73 - protein, 174 aa, synthetic construct
ILVCFVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 74 - protein, 174 aa, synthetic construct
ILVCFVIILTYTLYTDAYCVEYEESEEDRQQCSSSNFPASLPHMLRELRAAFGKVKTFFQMKDQLNSMLLTQSLLDDFKGYLGCQAFSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 75 - protein, 174 aa, synthetic construct
LVCVAIILTYTLYTDAYCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 76 - protein, 177 aa, synthetic construct
MGLRSGLTLQCLVILQCLVMLYLAPACKGVSNCGNLPHMLRDLRDAFSRVKTFFQMKDQLDNILLKESLLEDFKGYLGCQALSEMIQFYLEEVMPQAENQDPHAKEHVNSLGENLKTLRLRLRRCHRFLPCENKSKAVEQVKNAFSKLQEKGVYKAMSEFDIFINYIEAYMTMKIRR

SEQ ID NO: 77 - protein, 174 aa, synthetic construct
LVCVAIILTYTLYTDAYCVEYLESREDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 78 - protein, 174 aa, synthetic construct
ILVCFVIILTYTLYTDAYCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSPGEKLKTLRLRLRRCHRFLPCENKSKAVEQVKRVFNMLQERGVYKAMSEFDIFINYIES

SEQ ID NO: 79 - protein, 186 aa, synthetic construct
MANVIYVVLALNILLSQIHVSNPYCTSCSYRDCTEDEDQKQQCEGGLRSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 80 - protein, 174 aa, synthetic construct
ILVCFVIILTYTLYTDAYCVEYEESEEDRQQCSSSNFPASLPHMPRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 81 - protein, 189 aa, synthetic construct
MANVIYVVLALNILLSQFHVSNPYCTSCSHRDCTEDDEQKQQCEGGSGGLGSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 82 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPGAKDHVNSLGENLKTLRLRLRRCHRFLPCENKSKAVEQIKNAFNKLQEKGIYKAMSEFDIFINYIEAYMTIKAR

SEQ ID NO: 83 - protein, 170 aa, synthetic construct
MERRLVVTLQCLVLLYLAPECGSTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 84 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTMKAR

SEQ ID NO: 85 - protein, 171 aa, synthetic construct
MGPRAGLALQCLLLLYLAPACKGVSNCGNLPHMLRDLRDAFSRVKTFFQMKDQLDNILLKESLLEDFKGYLGCQALSEMIQFYLEEVMPQAENQDPNAKEHVNSLGENLKTLRLRLRRCHRFLPCENKSKAVEQVKNAFSKLQEKGVYKAMSEFDIFINYIEAYMTMKTRR

SEQ ID NO: 86 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTIRLRLRRCHRFLPCENKSKAVEQIKNAFNKLQEKGIYKAMSEFDIFINYIEAYMTIKAR

SEQ ID NO: 87 - protein, 170 aa, synthetic construct
MERRLVVTLQCLVLLYLAPECGGTDQCDNFPQMLRDLRDAFSRVKTFFQTKDEVDSLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 88 - protein, 170 aa, synthetic construct
MERRLMVTLQCLVLLYLAPECGSTDQCDNFPQMLRDLRDAFSRVKTFFQTKDEVDNILLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 89 - protein, 170 aa, synthetic construct
MERRLVVTLQCLVLLYLAPECGGTDQCDNFPQMLRDLRDAFSRVKTFFQTKDEVDNLFLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 90 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEKVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 91 - protein, 170 aa, synthetic construct
MERRLMVTLQCLVLLYLAPECGSTDQCDNFPQMLRDLRDAFSRVKTFFQTKDAVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 92 - protein, 177 aa, synthetic construct
MELRSGLTLQCLVMLQCLVMLYLAPACKGASNCGNLPHMLRDLRDAFSRVKTFFQMKDQLDNILLKESLLEDFRGYLGCQALSEMIQFYLEEVMPQAENQDPHSKEHVNSLGENLKTLRLRLRRCHRFLPCENKGKAVEQVKNAFSKLQEKGVYKAMSEFDIFINYIEAYMTMKLRR

SEQ ID NO: 93 - protein, 170 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFHIFINYIEAYMTIKAR

SEQ ID NO: 94 - protein, 161 aa, synthetic construct
MERRLVVTLQCLVLLYLAPECGEMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 95 - protein, 171 aa, synthetic construct
MELSLGLTLHFLVFLCLAPACGRAETCGNIPHMLRDLRDAFSRVKTFFQMKDQLDNILLKESLLEDFKGYLGCQALSEMIQFYLEEVMPQAEAMSLKSQEHVNFLGENLNTLRLRLRRCHRFLPCENKSKAVEQVKNAFSKLQEKGVYKAMSEFDIFINYIEAYMTMKLRR

SEQ ID NO: 96 - protein, 204 aa, synthetic construct
MAYGKKIVAASLLVIPAYVVFTNATANNRAQKCFCFDGSNAGNSEETNTAAFQKKCDSEIPESLPYMLRDLRNSSVQTRRYFQEKDEENSPLLTQKLLEDFKGYLGCQALSEMIQFYLEEVMPQAEDSNPSAKDSVTSLGEKLKTLRLRLRRCHRFLPCENKSKAVENLKSKFGDLGNQGVHKAMSEFDIFINYIETYMTTKMK

SEQ ID NO: 97 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLETVFPAGDHVYPGLKTELHSMRSTLESIYKDMRQCPLLGCGDKSVISRLSQEAERKSDNGTRKGLSELDTLFSRLEEYLHSRK

SEQ ID NO: 98 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 99 - protein, 175 aa, synthetic construct
MLSVMVSSSLVLIVFFLGASEEAKPATTTTKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 100 - protein, 176 aa, synthetic construct
MLSVMVSSSLVLIVFFLGASEEAKPATTTTIKNTKPQCRPEDYATRLQDLRVTFHRVKPTLQREDDYSVWLDGTMVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 101 - protein, 176 aa, synthetic construct
MLSVMVSSSLVLIVFFLGASEEAKPATTTTIKNTKPQCRPEDYATRLQDLRVTFHRVKSTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 102 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDMLFSRLEEYLHSRK

SEQ ID NO: 103 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVLPAGDHVYPGLKTELHSMRSTLESIYKDMRQCPLLGCGDKSVISRLSQEAERKSDNGTRKGLSELDTLFSRLEEYLHSRK

SEQ ID NO: 104 - protein, 176 aa, synthetic construct
MLSVMVSSSLVLIVFFLGASEEAKPAATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 105 - protein, 176 aa, synthetic construct
MLSVMVSSSLVLIVFFLGASEEAKPATTTTINNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 106 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMHSTLESIYKDMRQCPLLGCGDKSVISRLSQEAERKSDNGTRKGLSELDTLFSRLEEYLHSRK

SEQ ID NO: 107 - protein, 176 aa, synthetic construct
MLSVMVSSSLVLIIFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 108 - protein, 175 aa, synthetic construct; identical to SEQ ID NO: 41

SEQ ID NO: 109 - protein, 176 aa, synthetic construct
MLSVMVSSSLVLIVFFLGASEEAKPATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 110 - protein, 176 aa, synthetic construct
MLSVMVSSSLVLIVFFLGASEEAKPAATTTIKNTKPQCRPEDYATRLQDLRVTFHRVKPTLQHEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 111 - protein, 176 aa, synthetic construct
MLSVMVSSSLVLIVFFLGASEEAKPATTTTIKNTKPQCRPEDYATRLQDLRVTFHRVKPTLQREDDYSVWLDGMVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 112 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVVDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 113 - protein, 176 aa, synthetic construct
MLSVMVSSSLVLIVFFLGASEEAKPATTTTIKNTKPQCRPEDYATRLQDLRITFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 114 - protein, 175 aa, synthetic construct
MLSVMVSSSLVLIVFFLGASEEAKPATTTIKNTKPQCRPEDYATRLQDLRVTFHRIKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 115 - protein, 176 aa, synthetic construct
MLSVMVSSSLVLIVFFLGASEEAKPATTTTIKNTKPQCRPEDYATRLQDLRVTFHRVKPTLQCEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 116 - protein, 177 aa, synthetic construct
MLSVMVFSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 117 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 118 - protein, 176 aa, synthetic construct
MLSVMVSSSLVLIVFFLGASEEAKPATTTTIKNTKPQCRPEDYATRLQDLRVTFHRVKPTLQREDDYSVWLDGTVVKGCWGCSVMDWLLRRYLEILFPAGDHVYPGLKTELHSMRSTLESIYKDMRQCPLLGCGDKSVISRLSQEAERKSDNGTRKGLSELDTLFSRLEEYLHSRK

SEQ ID NO: 119 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 120 - protein, 175 aa, synthetic construct
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPRLKTELHSMRSTLESIYKDMQQCPLLGCGDKSVISRLSQEAERKSDNGTRKGLSELDTLFSRLEEYLHSRK

SEQ ID NO: 121 - protein, 177 aa, synthetic construct
MLSVMVSSSLVLIVFFLGASEEAKPAATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 122 - protein, 178 aa, synthetic construct
MLSVMVSSSLVLIVFFLGASEEAKPAATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 123 - protein, 177 aa, synthetic construct
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 124 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIMFPAGDHVYPGLKTELHSMRSTLESIYKDMRQCPLLGCGDKSVISRLSQEAERKSDNGTRKGLSELDTLFSRLEEYLHSRK

SEQ ID NO: 125 - protein, 175 aa, synthetic construct
MLSVMVSSSLVLIVFFLGASEEAKSATTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 126 - protein, 176 aa, synthetic construct
MLSVMVSSSLVLIIFFLGASEEAKPATTTTIKNTKPQCRPEDYATRLQDLRVTFHRVKPTLQREDDYSVWLDGTVVKGCWGCSVMDWLLRRYLEIVFPAGDYVYPGLKTELHSMRSTLESIYKDMRQCPLLGCGDKSVISRLSQEAERKSDNGMRKGLSELDTLFSRLEEYLHSRK

SEQ ID NO: 127 - protein, 175 aa, synthetic construct
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 128 - protein, 177 aa, synthetic construct
MLSVMVSSSLVLIVFFLGASEEAKPATTTTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 129 - protein, 178 aa, synthetic construct
MLSVMVSSSLVLIVFFLGASEEAKPAATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 130 - protein, 178 aa, synthetic construct
MLSVMVSSSLVLIIFFLGASEEAKPATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 131 - protein, 176 aa, synthetic construct
MLSVMVSSSLVLIVFFLGASEEAKPAATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLNELDTLFSRLEEYLHSRK

SEQ ID NO: 132 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 133 - protein, 176 aa, synthetic construct
MLSVMVSSSLVLIVFFLGASEEAKPATTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 134 - protein, 179 aa, synthetic construct
MLSVMVSSSLVLIVFFLGASEEAKPATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 135 - protein, 177 aa, synthetic construct
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKSLSELDTLFSRLEEYLHSRK

SEQ ID NO: 136 - protein, 175 aa, synthetic construct
MLSVMVSSSLVLIVFFLGASEKAKSATTTIKNTKPQCRPEDYATRLQDLRVTFHRVKPTLQREDDYSVWLDGTVVKGCWGCSVMDWLLRRYLEIVFPAGDHVYSGLKTELHSMRSTLESIYKDMRQCPLLGCGDKSVISRLSQEAERKSDNGTRKGLSELDTLFSRLEEYLHSRK

SEQ ID NO: 137 - protein, 179 aa, synthetic construct
MLSVMVSSSLVLIVFFLGASEEAKPAATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 138 - protein, 179 aa, synthetic construct
MLSVMVSSSLVLIVFFLGASEEAKPAATTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 139 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMWQCPLLGCGDKSVISRLSQEAERKSDNGTRKGLSELDTLFSRLEEYLHSRK

SEQ ID NO: 140 - protein, 177 aa, synthetic construct
MLSVMVSSSLVLIVFFLGASEEAKPAATTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 141 - protein, 173 aa, synthetic construct
MLSVMVSSSLVLIVFFLGASEEAKPATIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 142 - protein, 180 aa, synthetic construct
MLSVMVSSSLVLIVFFLGASEEAKPAATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 143 - protein, 180 aa, synthetic construct
MLSVMVSSSLVLIVFFLGASEEAKPAATTTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 144 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLRELDTLFSRLEEYLHSRK

SEQ ID NO: 145 - protein, 181 aa, synthetic construct
MLSVMVSSSLVLIVFFLGASEEAKPAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 146 - protein, 179 aa, synthetic construct
MLSVMVSSSLVLIVFFLGASEEAKPAATTTTTTIKNTKPQCRPEDYATRLQDLRVTFHRVKPTLQREDDYSVWLDGTVVKGCWRCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 147 - protein, 177 aa, synthetic construct
MLSVMVSSSLVMIVFFLGASEEAKPATTTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 148 - protein, 182 aa, synthetic construct
MLSVMVSSSLVLIVFFLGASEEAKPAAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 149 - protein, 180 aa, synthetic construct
MLSVMVSSSLVLIVFFLGASEEAKPAATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFIRLEEYLHSRK

SEQ ID NO: 150 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGRSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 151 - protein, 177 aa, synthetic construct
MLSVMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESICKDMRQRPLLGCGDKSVISRLSQEAERKSDNGTRKGLSELDTLFSRLEEYLHSRK

SEQ ID NO: 152 - protein, 182 aa, synthetic construct
MLSVMVSSSLVLIVFFLGASEEAKPAATTTTTTTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 153 - protein, 176 aa, synthetic construct
MLSVMVSSSLVLIVFFLGASEEAKPATIKNTKPQCRPEDYATRLQDLRVTFHRVKPTLPGHQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 154 - protein, 170 aa, synthetic construct
MKTNTKIILFCYVILSLYVFSCAIASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 155 - protein, 170 aa, synthetic construct
MKTNTKIILFCYVILSLYVFSCAIASAKKCNDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 156 - protein, 171 aa, synthetic construct
MKTNTKIILFCYVIFLSLYVFSCVVASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 157 - protein, 170 aa, synthetic construct
MKTNTKIILFCYVILSLYVFSCVVAYAKKCDDVSFDYILKDLRSEFSKIKSFVQNNDQENMMLLSQSMLNKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 158 - protein, 170 aa, synthetic construct
MKTSTKIILFCYVILSLYVFSCVVASAKKCDDVSFDYILKDLRSEFIKIKSFVQNNDQENMMLLSQSMLDKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 159 - protein, 168 aa, synthetic construct
MKTNTKIILFCYVILYVFSCTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 160 - protein, 170 aa, synthetic construct
MKTSTKIILFCYVILSLYVFSCVVASAKKCDDVSFDYILKDLRSEFIKIKSFVQNNDQENMMLLSQSMLDKLTSRIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 161 - protein, 168 aa, synthetic construct
MKTNTKIILFCYVILYLFSCTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 162 - protein, 174 aa, synthetic construct
MLSVMVSSSLVLIVFFLGASEEAKPATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKAVISRLSQEAERKSDNGTRKGLSELDTLFSRLEEYLHSRK

SEQ ID NO: 163 - protein, 168 aa, synthetic construct
MKTNTKIILFCYVILYLFSCTVASAKKCDDVSFDYILKDLRSEFSKIKSFVQNNDKENMMLLSQSMLDKLTRCIGCKSLSDMIKFYLNDVLPNAEKIEHIKNKITSIGEKLKSLKERLISCDFLHCENHDEIKAVKTIFNELKDKGIYKAMGEFDIFINHLEKYIVKK

SEQ ID NO: 164 - protein, 150 aa, synthetic construct
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKMPLTSCRKKEFTKP

SEQ ID NO: 165 - protein, 185 aa, synthetic construct
MGSRRLSRCSLATAVCLVAIVVAVAAKGRDSKPSPACDPMHGALAGIFKELRTTYRSVRETLQTKDTVYYVSLFHEQLLQEMLSPVGCRVTNELMQHYLDGVLPRAFHCGYDNTTLNALHFLSSSLSTLYQHMLKCPALACTGQTPAWTQFLDTEHKLDPWKGTVKATAEMDLLLNYLETFLLQS

SEQ ID NO: 166 - protein, 159 aa, synthetic construct
MLSVMVSSSLVLIVFLLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCEAERKSDNGTRKGLSELDTLFSRLEEYLHSRK

SEQ ID NO: 167 - protein, 187 aa, synthetic construct
MRRRRSFGVVVVGAIGTLLMMAVVVLSAHDHEHKVPPACDPVHGNLAGIFKELRTIYTSIREGLQKKDTVYYTSLFNDRVLQEMLSPMGCRVTNEIMEHYLDGVLPRASHLDYDNSTLNGLHAFASSMQALYQHMLKCPALACTGKTPAWMYFLEVEHKLNPWRGTAKAAAEADLLLNYLETFLLQF

SEQ ID NO: 168 - protein, 188 aa, synthetic construct
MGSRPARMCGLSNLLCLLLVVLVAVVIHRGCGASKPPVDCDPIHGTLSRIIKEVRTGYGSIKQALQSKDTVYYVSLFHENLLNEMLSPVGCRVTNELMQHYLDGVLPRAFQCGYDNTTLDGLHSLVSSLDALYKHMLKCPALACTGQTPAWTQFLETEHKLDPWKGTIKATAEMDLLVNYLETFLAQS

SEQ ID NO: 169 - protein, 142 aa, synthetic construct
MLSMMVSSSLVLIVFFLGASEEAKPATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISR

SEQ ID NO: 170 - protein, 139 aa, synthetic construct
MLSVMVSSSLVLIVFFLGASEEAKPATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCVSVSVAALSAQR

SEQ ID NO: 171 - protein, 190 aa, synthetic construct
MININILSLLILILSIYANAIIDTCYDDQERERTKSNSISSVTPEMCKGLKQLVSTKLKDARQKEKSVRDYFTSRDNDLDFMLLQGVKETHKKTCGCYVLYLLLSFYGKTIRDTIQSNKHKNLNTELTNLAVSVLSLEDLLEACGITCNPKKDSLLKRIEEYMKEHGDDAIYKVIGEIEFLFQAIEKHVY

SEQ ID NO: 172 - protein, 192 aa, synthetic construct
MINISINILSLLILILSIYANSIIDMCYDDQERERTKSNSISSITPDMCKGLKQLVATKLKDARQKEKLVNSYFTSRDNDLTYMLLQGVRETHKKPCGCYVLYLLLTFYRKTIKDTIQSKKHESINTELTNLAVTVLSLEDLLEACGITCNPKKDSLLKRIEGYTKEHGDDAIYKVIGEIDFLFQAIERHVY

SEQ ID NO: 173 - protein, 191 aa, synthetic construct
MININILSLLILILSIYANAIIDTCYDDQERERTKSNSISSVTPEMCKGLKQLVATKLKDARQKEKLVNDYFTGRDNDLSYMLLQGVRETHKKPCGCYVLYLLLSFYRKTIRDTIQSNKHASINAELTNLAVSVLSLEDLLDACGITCNPKKDSLLKRIEEYMKEHGDDAIYKLIGEIEFLFQAIERHVYT

SEQ ID NO: 174 - protein, 191 aa, synthetic construct
MININILSLLILILSIYANAIIDTYDEDEDEDSIKLSSIGSITPEMCKNLKQLVASKLKDIRQKEKSLRDYFTNLDDELDYMLLQGVGENHKKKCGCYILHLLLKFYSKTIRNTIQSEKHKNVNLELTNIAVSMLALEDLLEKCGITCNPKKDPLLKRIEDYMKQHGDDGVNKAIAELEFLFQMIEKQVYI

SEQ ID NO: 175 - protein, 165 aa, synthetic construct
MARFIYVVLLCLVFDAAQSAAQCRKGTITSRLKMLRTAFEKVREFYEDRDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 176 - protein, 170 aa, synthetic construct
MMLALAMMLMALGPLSTNAMYVQHGSDYCTTTVHADIASAISGMRAEYDSGLGHYFKSLVPHPDNPYDTDDYKYMINNTNSYNCHALQSTINALLGMYGYVDIDEPHQLAMMKLATHTMQAAMLLNKCAKQLGCYHIPFDVETLHEAHPDDVMASLDTALNLMSMVTNEI

SEQ ID NO: 177 - protein, 165 aa, synthetic construct
MARFIYVVLLCLVFDAAQSAAQCRKGTITSRLKMLRTAFEKVREFYEDSDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 178 - protein, 168 aa, synthetic construct
MFQLEGIVLLVYLANWVSPATIKCVGMSTLFNPELIQLRRLFGDGIKDFFQNKDEDLDNAFLNEDVQRELASDCGCDHLMDMLSLYVNDTIPKGMKTEDAPSGLGQMGQLMSSLYRKMDMCWSELGCSHNTRLTLQEYADKKGGWDNKALGESDILFDALELFFSKIK

SEQ ID NO: 179 - protein, 169 aa, synthetic construct
MMTTLALVMTLMALSTNAMYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPNDVMASLDTALNLMSMVTNEI

SEQ ID NO: 180 - protein, 171 aa, synthetic construct
MITLALVMTLMALGPLSTNAMYARRSGDYCTTTVRADIASAISGMRAEYNSGLRDYFKSLVPHPDNPYDTDDYKYMLNNTNSYNCHALQSTINALLGMYGYVDIDESHQLAMMKLATHTMQTAMMLNKCAAQLGCYHIPFDLETLHEAHPDDVMASLDTALNLMSMITNEI

SEQ ID NO: 181 - protein, 169 aa, synthetic construct
MMTTLALVMTLMALATNAMYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPDDVMASLDTALNLMSMVTNEI

SEQ ID NO: 182 - protein, 177 aa, synthetic construct
MSGTSNKKFVFLVAIAVAICMMSSVSSNVHSGTEDNPCTNSKTVLNTLLNQIKQEYINNLLPYYKALTPKPVDVFDDSYTYSIQSTDYNCYTIYETLNFLLGDVFPRATTDATVRLSLAKIATSSQQASMLMNLCKKELACGPAPFDMIKLYHDTKEYGADNIMGTLDTPFQYFVIV

SEQ ID NO: 183 - protein, 170 aa, synthetic construct
MLALAMVLMALGPLSTNAMYVQHGRGDYCTTTVRADIASAISGMRAEYDSGLGHYFKSLVPHPDNPYDTDDYKYMINNTNSYNCHALQSTINALLGMYGYVDIDEPHQLAMMKLATHTMQTAMLLNKCAEQLGCYHIPFDVEILHEAHPDDVMASLDTALNLMSMVTNEI

SEQ ID NO: 184 - protein, 179 aa, synthetic construct
MFLAVLLTATIFFEARGAPATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCRDFLKCEDLPHWHQMAENYKEKPMQGFSEMDFVFQSVEKFLVAKDVKNMKTKRKH

SEQ ID NO: 185 - protein, 170 aa, synthetic construct
MLKQIIVVCIVAMAAVFADDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCRQQLGCGPPPFDAKTLYDDRAEYGADDIMATLDTALAKFKLVLESENVV

SEQ ID NO: 186 - protein, 170 aa, synthetic construct
MLKQIIVVCIVAMAAVFADDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCREQLGCGPPPFDAKTLYDDRAEYGADDIMATLDTALAKFKLVLESENVV

SEQ ID NO: 187 - protein, 171 aa, synthetic construct
MITLALVMTLMALGPLSTNGVHARRRGDYCTTTVRADIASAISGMRAEYNSGLGDYFKSLVPHPDNPYDTDDYKHMIDNANSYNCHALQSTINALLGMYGYVDIDEPHQLAMMKLATHTMQTAMLLNKCAAQLGCYHIPFDLETLREAPPADVMASLDTALNLMSMITNEI

SEQ ID NO: 188 - protein, 180 aa, synthetic construct
MDAQFLLLIVLALPASFAASLSTHYNNYDLTRIATIDKDVCKRVAQHINDDFVNMRKLYETQLKNYFQQLVPNPTDVFKDDSYMYMINGTDYNCHIIYETMRFLSGDVFPFATETEAELQYMWKMMLGVSQLSAYIGNCYQYFKCGPAPFDPQVLYHDRELFHADTVMAYLDTAFSHFTL

SEQ ID NO: 189 - protein, 156 aa, synthetic construct
MKLYFYCIFFYKIIVTISLNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNTVNVLEIKKLLLIDNNCKKLFSEIDIFLTWVMAKI

SEQ ID NO: 190 - protein, 156 aa, synthetic construct
MKLYFYCIFFYKIIVTISLNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNNVNVLEIKKLLLIDNNCKKLFSEIDIFLTWVMAKI

SEQ ID NO: 191 - protein, 134 aa, synthetic construct
MFLAVLLTATIFFEARGAPATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCVSRTQHLCYD

SEQ ID NO: 192 - protein, 179 aa, synthetic construct
MFRASLLCCLVLLAGVWADNKYDSESGDDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 193 - protein, 179 aa, synthetic construct
MFRASLLCCLVLLAGVWADNKYDSESGNDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 194 - protein, 179 aa, synthetic construct
MFRASLLCCLVLLAGVWADNKYDSESGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 195 - protein, 179 aa, synthetic construct
MFRASLLCCLVLLAGVWADNKYDSESGDDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 196 - protein, 179 aa, synthetic construct
MFRALLLCCLALLAGVWADNRYDGQDGNDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 197 - protein, 175 aa, synthetic construct
MLSVMVSSSLVLIVFFLGASEEAKPATTAIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 198 - protein, 176 aa, synthetic construct
MLSVMVSSSLVLIVFFLGAFEEAKPATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 199 - protein, 177 aa, synthetic construct
MLSVMVSSSLVLIVFFLGASEEAKPATTTTTIKNTKPQCRPEDYATRLQDLRVTFYRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 200 - protein, 174 aa, synthetic construct
MLSVMVSSSLVLIVFFLGASEEAKPATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 201 - protein, 171 aa, synthetic construct
MKTNTKIILFCYVIFLSLYVFSCVVASTKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 202 - protein, 148 aa, synthetic construct
MLSVMVSSSLVLIVFFLGASEEAKPATTTIKNTKPQCRPEDYATRLQDLRVTFHRVKPTLQREDDYSVWLDGDHVYPGLKTELHSMRSTLESIYKDMRQCPLLGCGDKSVISRLSQEAERKSDNGTRKGLSELDTLFSRLEEYLHSRK

SEQ ID NO: 203 - protein, 190 aa, synthetic construct
MSNFRILSLLIFSLIVHVNAMIGTCYDEDEEIERLKSNSISSITPGMCRNLKHSVMIRLIDARQIEASIRSYFTDGDNNLSFMLLQGIREISKKKCGCYILNLMLRFYIQTIKHTILSNKHKDMNLELTNLAVTILSLESLLEKCGVTCNPVKDPLLTRIEEYTRKHGDNAIYKTIGELEFLFDAIEKFV

SEQ ID NO: 204 - protein, 165 aa, synthetic construct
MARFIYVVLLCLVFDAAQSAAQCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVIGM

SEQ ID NO: 205 - protein, 165 aa, synthetic construct
MARFIYVVLLCLVFDAAQSAAQCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVNGM

SEQ ID NO: 206 - protein, 165 aa, synthetic construct
MARFIYVVLLCLVFDAAQSAAQCRKGTITIRLKMLRTAFEKVREFYEDRDEEETALASTEHLHGPESCSVIDELITHYTKCVIPAANEEEGADLLSLDTLQFALENVKGLLANCQEEFGCKPPFSMRDYKKQYRQLNKEKNAGMIKAMGELGMLFNGIEERVIGM

SEQ ID NO: 207 - protein, 165 aa, synthetic construct
MARFIYVVLLCLVFDAAQSAAQCRKGTITSRLKMLRTAFEKVREFYEDRDEEETALASTEHLHGPESCSVIDELITHHTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 208 - protein, 125 aa, synthetic construct
MLSVMVSSSLVLIVFLLGASEEAKPATTTIKNTKPQCRPEDYATRLQDLRVTFHRVKPTLVGHVGDHVYPGLKTELHSMRSTLESIYKDMRQCEAERKSDNGTRKGLSELDTLFSRLEEYLHSRK

SEQ ID NO: 209 - protein, 178 aa, synthetic construct; identical to SEQ ID NO: 1

SEQ ID NO: 210 - protein, 178 aa, synthetic construct
MHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 211 - protein, 178 aa, synthetic construct
MHSSALLCCLVLLTGVRASPGQGTQSENSCTHFPGNLPNMLRALRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 212 - protein, 154 aa, synthetic construct
RHCMFGDSLRNSPDMKNMLQDLRGGYSGSGIKRTFQGKDTLDSMLLTQSLLDDFKGYLGCQALSEMIQFYLEEVMPQAENHGPTDSVKQLGEKLHTLNQKFGECPRWFPCYYNTTPAVENVKSVFSKLQERGVYKAMSEFDIFINYIETYTTMK

SEQ ID NO: 213 - protein, 171 aa, synthetic construct
CQLESGEALPLGSRSADSRSVDGQRVPAPQNNYPGLLRDLRLGYEGFKQKVTDSHPDETLLGSSRLAGDLKGPLRCQALSEMIQFLLQVVLPDAENSRQDLRSQFSTLGDRITGLRQQLRRDPTVFPCESRSDGVSDLRSAYTRLGSTGAEKVLSEFDIFINYIEAYVTSV

SEQ ID NO: 214 - protein, 164 aa, synthetic construct
YCVEYAESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 215 - protein, 176 aa, synthetic construct
YCTSCSHHQCTEDENQKQDCEDANHSLPHMLRELRAAFGKVKTFFQMKDQLHSLLLTQSLLDDFKGYLGCQALSEMIQFYLEEVMPQAENHGPEEHDNSLSEHGPDVKEHVNSLGEKLKTLRLRLRRCHRFLPCENKSKAVEKVKRVFSELQERGVYKAMSEFDIFINYIETYMTT

SEQ ID NO: 216 - protein, 161 aa, synthetic construct
RSPKNKPSIDCNPQTGDFVNMLKSMRQDYSRIRDTLHDRDKLHSSLLTGALLDEMMGYSGCRTTLLLMEHYLDTWYPAAYRHHLYDNQTLVVVDRMGSTLVALLKAMVQCPMLACGAPSPAMDKMLQQEAKMKKYTGVYKGISETDLLLGYLELYMMKFKR

SEQ ID NO: 217 - protein, 157 aa, synthetic construct
HEHKVPPACDPVHGNLAGIFKELRAIYASIREALQKKDTVYYTSLFNDRVLQEMLSPMGCRVTNELMEHYLDGVLPRAAHFDYDNSTLNGLHAFTSSMQALYQHMLKCPALACTGKTPAWMYFLEVEHKLNPWRGTAKAAAEADLLLNYLETFLLQF

SEQ ID NO: 218 - protein, 159 aa, synthetic construct
KGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 219 - protein, 150 aa, synthetic construct
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 220 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKP

SEQ ID NO: 221 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKT

SEQ ID NO: 222 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKS

SEQ ID NO: 223 - protein, 162 aa, synthetic construct
YCVEYKESEEDRQQCSSSSFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTK

SEQ ID NO: 224 - protein, 163 aa, synthetic construct
YCVEYAESDEDKQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 225 - protein, 163 aa, synthetic construct
YCVEYEESEEDKQQCGSNGGPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 226 - protein, 163 aa, synthetic construct
YCVEYAESDEDRQQCSGSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 227 - protein, 164 aa, synthetic construct
YCVEYEESEEDKQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 228 - protein, 163 aa, synthetic construct
YCIQYEESEEDKQQCSSSNFPASLPHMLRELRAAFGKVKTFFQMKDQLNSMLLTQSLLDDFKGYLGCQALSEMIRFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 229 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLREKLKTLRLRLRRCHRFLPCENKSKAVEQVKRVFNMLQERGVYKAMSEFDIFINYIESYMTTKT

SEQ ID NO: 230 - protein, 164 aa, synthetic construct
YCVEYEESEEDKQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 231 - protein, 163 aa, synthetic construct
YCVEYAESDEDKQQCSGSNFPASLPHMLRELRAAFGKVKTFFQMKDQLNSMLLTQSLLDDFKGYLGCQALSEMIQFYLEEVMPRAENHGPDIKEHVNSLGEKLKTLRLRLRRCHRFLPCENKSKAVEQVKRVFNMLQERGVYKAMSEFDIFINYIESYMTTKM

SEQ ID NO: 232 - protein, 164 aa, synthetic construct
YCVEYEESEEDRQQCSGSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 233 - protein, 164 aa, synthetic construct
YCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 234 - protein, 164 aa, synthetic construct
YCVEYEESDEDKQQCSSSTGAPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 235 - protein, 164 aa, synthetic construct
YCVEYEETKEDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 236 - protein, 163 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGDYKAMSEFDIFINYIESYMTTKS

SEQ ID NO: 237 - protein, 164 aa, synthetic construct
YCVEYEESEEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCEDKSKAVEQVKRVFNMLQERGVYKAMSEFDILINYIESYMTTKM

SEQ ID NO: 238 - protein, 164 aa, synthetic construct
YCVEYEESDEDRQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMATKM

SEQ ID NO: 239 - protein, 161 aa, synthetic construct
DNRYDGQDGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 240 - protein, 164 aa, synthetic construct
YCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 241 - protein, 164 aa, synthetic construct
YCVEYEESKEDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 242 - protein, 164 aa, synthetic construct
YCVEYLESGEDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 243 - protein, 164 aa, synthetic construct
YCVEYLESREDEQQCSGSNGASASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 244 - protein, 160 aa, synthetic construct
ESENNCTHFPTSLPHMLHELRAAFSRVKTFFQMKDQLDNMLLNGSLLEDFKGYLGCQALSEMIQFYLEEVMPQAENHSGGGGPDIKEHVNSLGEKLKTLRVRLRRCHRFLPCENKSKAVEQVKSAFSKLQEKGVYKAMSEFDIFINYIEAYMTTKMKNKK

SEQ ID NO: 245 - protein, 164 aa, synthetic construct
YCVEYLESDEDKQHCSSSNGASASSPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIESYMTTKM

SEQ ID NO: 246 - protein, 163 aa, synthetic construct
YCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLAEKLKTLRLRLRRCHRFLPCENKSKAVEQVKRVFNMLQERGVYKAMSEFDIFINYIESYMTTKS

SEQ ID NO: 247 - protein, 163 aa, synthetic construct
YCVEYLESEEDKQQCGSNGASSSSPHMLRELRAAFGKVKTFFQMKDQLNSMLLTQSLLDDFKGYLGCQALSEMIQFYLEEVMPQAENHGPDVKEHVNSLGEKLKTLRLRLRRCHRFLPCENKSKAVAQVKRVFNMLQERGVYKAMSEFDIFINYIESYMTTKS

SEQ ID NO: 248 - protein, 157 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 249 - protein, 157 aa, synthetic construct
YCVEYEESEEDRQQCSSSNFPASLPHMLRELRAAFGKVKTFFQMKDQLNSMLLTQSLLDDFKGYLGCQAFSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 250 - protein, 158 aa, synthetic construct
YCVEYLESREDEQQCSSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 251 - protein, 148 aa, synthetic construct
VSNCGNLPHMLRDLRDAFSRVKTFFQMKDQLDNILLKESLLEDFKGYLGCQALSEMIQFYLEEVMPQAENQDPHAKEHVNSLGENLKTLRLRLRRCHRFLPCENKSKAVEQVKNAFSKLQEKGVYKAMSEFDIFINYIEAYMTMKIRR

SEQ ID NO: 252 - protein, 158 aa, synthetic construct
YCVEYLESREDEQQCGSSSNFPASLPHMLRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 253 - protein, 157 aa, synthetic construct
YCVEYEESEEDRQQCSSSNFPASLPHMLRELRAAFGKVKTFFQMKDQLNSMLLTQSLLDDFKGYLGCQALSEMIQFYLEEVMPQAENHGPDIKEHVNSPGEKLKTLRLRLRRCHRFLPCENKSKAVEQVKRVFNMLQERGVYKAMSEFDIFINYIES

SEQ ID NO: 254 - protein, 163 aa, synthetic construct
YCTSCSYRDCTEDEDQKQQCEGGLRSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 255 - protein, 157 aa, synthetic construct
YCVEYEESEEDRQQCSSSNFPASLPHMPRELRAAFGKVKTFFQMKDQLNSMLLTQSLLDDFKGYLGCQALSEMIQFYLEEVMPQAENHGPDIKEHVNSLGEKLKTLRLRLRRCHRFLPCENKSKAVEQVKRVFNMLQERGVYKAMSEFDIFINYIES

SEQ ID NO: 256 - protein, 166 aa, synthetic construct
YCTSCSHRDCTEDDEQKQQCEGGSGGLGSLPHMLRELRAAFGKVKTFFQMKDQLHSLLLTQSLLDDFKGYLGCQALSEMIQFYLEEVMPQAENHGPDIKEHVNSLGEKLKTLRLRLRRCHRFLPCENKSKAVEKVKRVFSELQERGVYKAMSEFDIFINYIETYMT

SEQ ID NO: 257 - protein, 147 aa, synthetic construct
TDQCDNFPQMLRDLRDAFSRVKTFFQTKDEVDNLLLKESLLEDFKGYLGCQALSEMIQFYLEEVMPQAENQDPGAKDHVNSLGENLKTLRLRLRRCHRFLPCENKSKAVEQIKNAFNKLQEKGIYKAMSEFDIFINYIEAYMTIKAR

SEQ ID NO: 258 - protein, 147 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 259 - protein, 147 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTMKAR

SEQ ID NO: 260 - protein, 148 aa, synthetic construct
VSNCGNLPHMLRDLRDAFSRVKTFFQMKDQLDNILLKESLLEDFKGYLGCQALSEMIQFYLEEVMPQAENQDPNAKEHVNSLGENLKTLRLRLRRCHRFLPCENKSKAVEQVKNAFSKLQEKGVYKAMSEFDIFINYIEAYMTMKTRR

SEQ ID NO: 261 - protein, 147 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTIRLRLRRCHRFLPCENKSKAVEQIKNAFNKLQEKGIYKAMSEFDIFINYIEAYMTIKAR

SEQ ID NO: 262 - protein, 147 aa, synthetic construct
TDQCDNFPQMLRDLRDAFSRVKTFFQTKDEVDSLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 263 - protein, 147 aa, synthetic construct
TDQCDNFPQMLRDLRDAFSRVKTFFQTKDEVDNILLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 264 - protein, 147 aa, synthetic construct
TDQCDNFPQMLRDLRDAFSRVKTFFQTKDEVDNLFLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 265 - protein, 147 aa, synthetic construct
TDQCDNFPQMLRDLRDAFSRVKTFFQTKDEVDNLLLKESLLEDFKGYLGCQALSEMIQFYLEKVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 266 - protein, 147 aa, synthetic construct
TDQCDNFPQMLRDLRDAFSRVKTFFQTKDAVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 267 - protein, 148 aa, synthetic construct
ASNCGNLPHMLRDLRDAFSRVKTFFQMKDQLDNILLKESLLEDFRGYLGCQALSEMIQFYLEEVMPQAENQDPHSKEHVNSLGENLKTLRLRLRRCHRFLPCENKGKAVEQVKNAFSKLQEKGVYKAMSEFDIFINYIEAYMTMKLRR

SEQ ID NO: 268 - protein, 147 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFHIFINYIEAYMTIKAR

SEQ ID NO: 269 - protein, 139 aa, synthetic construct
EMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 270 - protein, 147 aa, synthetic construct
ETCGNIPHMLRDLRDAFSRVKTFFQMKDQLDNILLKESLLEDFKGYLGCQALSEMIQFYLEEVMPQAEAMSLKSQEHVNFLGENLNTLRLRLRRCHRFLPCENKSKAVEQVKNAFSKLQEKGVYKAMSEFDIFINYIEAYMTMKLRR

SEQ ID NO: 271 - protein, 180 aa, synthetic construct
TANNRAQKCFCFDGSNAGNSEETNTAAFQKKCDSEIPESLPYMLRDLRNSSVQTRRYFQEKDEENSPLLTQKLLEDFKGYLGCQALSEMIQFYLEEVMPQAEDSNPSAKDSVTSLGEKLKTLRLRLRRCHRFLPCENKSKAVENLKSKFGDLGNQGVHKAMSEFDIFINYIETYMTTKMK

SEQ ID NO: 272 - protein, 151 aa, synthetic construct
ATTTTIKNTKPQCRPEDYATRLQDLRVTFHRVKPTLQREDDYSVWLDGTVVKGCWGCSVMDWLLRRYLETVFPAGDHVYPGLKTELHSMRSTLESIYKDMRQCPLLGCGDKSVISRLSQEAERKSDNGTRKGLSELDTLFSRLEEYLHSRK

SEQ ID NO: 273 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 274 - protein, 150 aa, synthetic construct
ATTTTKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 275 - protein, 151 aa, synthetic construct
ATTTTIKNTKPQCRPEDYATRLQDLRVTFHRVKPTLQREDDYSVWLDGTMVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 276 - protein, 151 aa, synthetic construct
ATTTTIKNTKPQCRPEDYATRLQDLRVTFHRVKSTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 277 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDMLFSRLEEYLHSRK

SEQ ID NO: 278 - protein, 151 aa, synthetic construct
ATTTTIKNTKPQCRPEDYATRLQDLRVTFHRVKPTLQREDDYSVWLDGTVVKGCWGCSVMDWLLRRYLEIVLPAGDHVYPGLKTELHSMRSTLESIYKDMRQCPLLGCGDKSVISRLSQEAERKSDNGTRKGLSELDTLFSRLEEYLHSRK

SEQ ID NO: 279 - protein, 151 aa, synthetic construct
AATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 280 - protein, 151 aa, synthetic construct
ATTTTINNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 281 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMHSTLESIYKDMRQCPLLGCGDKSVISRLSQEAERKSDNGTRKGLSELDTLFSRLEEYLHSRK

SEQ ID NO: 282 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 283 - protein, 150 aa, synthetic construct; identical to SEQ ID NO: 219

SEQ ID NO: 284 - protein, 151 aa, synthetic construct
ATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 285 - protein, 151 aa, synthetic construct
AATTTIKNTKPQCRPEDYATRLQDLRVTFHRVKPTLQHEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 286 - protein, 151 aa, synthetic construct
ATTTTIKNTKPQCRPEDYATRLQDLRVTFHRVKPTLQREDDYSVWLDGMVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 287 - protein, 151 aa, synthetic construct
ATTTTIKNTKPQCRPEDYATRLQDLRVTFHRVKPTLQREDDYSVWLDGTVVKGCWGCSVVDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 288 - protein, 151 aa, synthetic construct
ATTTTIKNTKPQCRPEDYATRLQDLRITFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 289 - protein, 150 aa, synthetic construct
ATTTIKNTKPQCRPEDYATRLQDLRVTFHRIKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 290 - protein, 151 aa, synthetic construct
ATTTTIKNTKPQCRPEDYATRLQDLRVTFHRVKPTLQCEDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 291 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 292 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 293 - protein, 151 aa, synthetic construct
ATTTTIKNTKPQCRPEDYATRLQDLRVTFHRVKPTLQREDDYSVWLDGTVVKGCWGCSVMDWLLRRYLEILFPAGDHVYPGLKTELHSMRSTLESIYKDMRQCPLLGCGDKSVISRLSQEAERKSDNGTRKGLSELDTLFSRLEEYLHSRK

SEQ ID NO: 294 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGMRKGLSELDTLFSRLEEYLHSRK

SEQ ID NO: 295 - protein, 150 aa, synthetic construct
ATTTIKNTKPQCRPEDYATRLQDLRVTFHRVKPTLQREDDYSVWLDGTVVKGCWGCSVMDWLLRRYLEIVFPAGDHVYPRLKTELHSMRSTLESIYKDMQQCPLLGCGDKSVISRLSQEAERKSDNGTRKGLSELDTLFSRLEEYLHSRK

SEQ ID NO: 296 - protein, 152 aa, synthetic construct
AATTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 297 - protein, 153 aa, synthetic construct
AATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 298 - protein, 152 aa, synthetic construct
AATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 299 - protein, 152 aa, synthetic construct
ATTTTTIKNTKPQCRPEDYATRLQDLRVTFHRVKPTLQREDDYSVWLDGTVVKGCWGCSVMDWLLRRYLEIMFPAGDHVYPGLKTELHSMRSTLESIYKDMRQCPLLGCGDKSVISRLSQEAERKSDNGTRKGLSELDTLFSRLEEYLHSRK

SEQ ID NO: 300 - protein, 150 aa, synthetic construct
ATTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 301 - protein, 151 aa, synthetic construct
ATTTTIKNTKPQCRPEDYATRLQDLRVTFHRVKPTLQREDDYSVWLDGTVVKGCWGCSVMDWLLRRYLEIVFPAGDYVYPGLKTELHSMRSTLESIYKDMRQCPLLGCGDKSVISRLSQEAERKSDNGMRKGLSELDTLFSRLEEYLHSRK

SEQ ID NO: 302 - protein, 150 aa, synthetic construct
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 303 - protein, 152 aa, synthetic construct
ATTTTTIKNTKPRCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 304 - protein, 153 aa, synthetic construct
AATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 305 - protein, 153 aa, synthetic construct
ATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 306 - protein, 151 aa, synthetic construct
AATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLNELDTLFSRLEEYLHSRK

SEQ ID NO: 307 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDALFSRLEEYLHSRK

SEQ ID NO: 308 - protein, 151 aa, synthetic construct
ATTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 309 - protein, 154 aa, synthetic construct
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 310 - protein, 152 aa, synthetic construct
AATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKSLSELDTLFSRLEEYLHSRK

SEQ ID NO: 311 - protein, 150 aa, synthetic construct
ATTTIKNTKPQCRPEDYATRLQDLRVTFHRVKPTLQREDDYSVWLDGTVVKGCWGCSVMDWLLRRYLEIVFPAGDHVYSGLKTELHSMRSTLESIYKDMRQCPLLGCGDKSVISRLSQEAERKSDNGTRKGLSELDTLFSRLEEYLHSRK

SEQ ID NO: 312 - protein, 153 aa, synthetic construct
ATTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQKAERKSDNGTRKGLSELDTLFSRLEEYLHSRK

SEQ ID NO: 313 - protein, 153 aa, synthetic construct
ATTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 314 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMWQCPLLGCGDKSVISRLSQEAERKSDNGTRKGLSELDTLFSRLEEYLHSRK

SEQ ID NO: 315 - protein, 152 aa, synthetic construct
AATTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 316 - protein, 148 aa, synthetic construct
ATIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 317 - protein, 154 aa, synthetic construct
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 318 - protein, 154 aa, synthetic construct
ATTTTTTMIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 319 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLRELDTLFSRLEEYLHSRK

SEQ ID NO: 320 - protein, 159 aa, synthetic construct
AKPAATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 321 - protein, 153 aa, synthetic construct
ATTTTTTIKNTKPQCRPEDYATRLQDLRVTFHRVKPTLQREDDYSVWLDGTVVKGCWRCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 322 - protein, 152 aa, synthetic construct
ATTTTTIKNTKPQCRPEDYATRLQDLCVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 323 - protein, 155 aa, synthetic construct
ATTTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 324 - protein, 154 aa, synthetic construct
ATTTTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFIRLEEYLHSRK

SEQ ID NO: 325 - protein, 152 aa, synthetic construct
ATTTTTIKNTKPQCRPEDYATRLQDLRVTFHRVKPTLQREDDYSVWLDGTVVKGCWGRSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 326 - protein, 152 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESICKDMRQRPLLGCGDKSVISRLSQEAERKSDNGTRKGLSELDTLFSRLEEYLHSRK

SEQ ID NO: 327 - protein, 160 aa, synthetic construct
AKPAATTTTTTTTTIKNTKPQCRPEDYATRLQDFRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 328 - protein, 151 aa, synthetic construct
ATIKNTKPQCRPEDYATRLQDLRVTFHRVKPTLPGHQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 329 - protein, 149 aa, synthetic construct
CAIASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 330 - protein, 149 aa, synthetic construct
CAIASAKKCNDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 331 - protein, 149 aa, synthetic construct
CVVASAKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 332 - protein, 149 aa, synthetic construct
CVVAYAKKCDDVSFDYILKDLRSEFSKIKSFVQNNDQENMMLLSQSMLNKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 333 - protein, 149 aa, synthetic construct
CVVASAKKCDDVSFDYILKDLRSEFIKIKSFVQNNDQENMMLLSQSMLDKLTSCIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 334 - protein, 143 aa, synthetic construct
KKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 335 - protein, 149 aa, synthetic construct
CVVASAKKCDDVSFDYILKDLRSEFIKIKSFVQNNDQENMMLLSQSMLDKLTSRIGCKSLSDMIKFYLNDVLPNAEKIEQIKNIITSIGEKLKSLKEKLISCDFLHCENNDEIKTVKAIFNKLKDKGIYKAMGEFDIFINYVEKYIVKT

SEQ ID NO: 336 - protein, 149 aa, synthetic construct
CTVASAKKCDDVSFDYILKDLRSEFSKIKSFVQNNDKENMMLLSQSMLDKLTSCIGCKSLSDMIKFYLNDVLPNAEKIEHIKNKITSIGEKLKSLKEKLISCDFLHCENHDEIKAVKTIFNKLKDKGIYKAMGEFDIFINHLEKYIVKK

SEQ ID NO: 337 - protein, 149 aa, synthetic construct
ATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKAVISRLSQEAERKSDNGTRKGLSELDTLFSRLEEYLHSRK

SEQ ID NO: 338 - protein, 149 aa, synthetic construct
CTVASAKKCDDVSFDYILKDLRSEFSKIKSFVQNNDKENMMLLSQSMLDKLTRCIGCKSLSDMIKFYLNDVLPNAEKIEHIKNKITSIGEKLKSLKERLISCDFLHCENHDEIKAVKTIFNELKDKGIYKAMGEFDIFINHLEKYIVKK

SEQ ID NO: 339 - protein, 127 aa, synthetic construct
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKMPLTSCRKKEFTKP

SEQ ID NO: 340 - protein, 159 aa, synthetic construct
KGRDSKPSPACDPMHGALAGIFKELRTTYRSVRETLQTKDTVYYVSLFHEQLLQEMLSPVGCRVTNELMQHYLDGVLPRAFHCGYDNTTLNALHFLSSSLSTLYQHMLKCPALACTGQTPAWTQFLDTEHKLDPWKGTVKATAEMDLLLNYLETFLLQS

SEQ ID NO: 341 - protein, 134 aa, synthetic construct
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCEAERKSDNGTRKGLSELDTLFSRLEEYLHSRK

SEQ ID NO: 342 - protein, 160 aa, synthetic construct
AHDHEHKVPPACDPVHGNLAGIFKELRTIYTSIREGLQKKDTVYYTSLFNDRVLQEMLSPMGCRVTNEIMEHYLDGVLPRASHLDYDNSTLNGLHAFASSMQALYQHMLKCPALACTGKTPAWMYFLEVEHKLNPWRGTAKAAAEADLLLNYLETFLLQF

SEQ ID NO: 343 - protein, 156 aa, synthetic construct
ASKPPVDCDPIHGTLSRIIKEVRTGYGSIKQALQSKDTVYYVSLFHENLLNEMLSPVGCRVTNELMQHYLDGVLPRAFQCGYDNTTLDGLHSLVSSLDALYKHMLKCPALACTGQTPAWTQFLETEHKLDPWKGTIKATAEMDLLVNYLETFLAQS

SEQ ID NO: 344 - protein, 117 aa, synthetic construct
ATTTTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISR

SEQ ID NO: 345 - protein, 114 aa, synthetic construct
ATTTIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCVSVSVAALSAQR

SEQ ID NO: 346 - protein, 170 aa, synthetic construct
IIDTCYDDQERERTKSNSISSVTPEMCKGLKQLVSTKLKDARQKEKSVRDYFTSRDNDLDFMLLQGVKETHKKTCGCYVLYLLLSFYGKTIRDTIQSNKHKNLNTELTNLAVSVLSLEDLLEACGITCNPKKDSLLKRIEEYMKEHGDDAIYKVIGEIEFLFQAIEKHVY

SEQ ID NO: 347 - protein, 172 aa, synthetic construct
NSIIDMCYDDQERERTKSNSISSITPDMCKGLKQLVATKLKDARQKEKLVNSYFTSRDNDLTYMLLQGVRETHKKPCGCYVLYLLLTFYRKTIKDTIQSKKHESINTELTNLAVTVLSLEDLLEACGITCNPKKDSLLKRIEGYTKEHGDDAIYKVIGEIDFLFQAIERHVY

SEQ ID NO: 348 - protein, 171 aa, synthetic construct
IIDTCYDDQERERTKSNSISSVTPEMCKGLKQLVATKLKDARQKEKLVNDYFTGRDNDLSYMLLQGVRETHKKPCGCYVLYLLLSFYRKTIRDTIQSNKHASINAELTNLAVSVLSLEDLLDACGITCNPKKDSLLKRIEEYMKEHGDDAIYKLIGEIEFLFQAIERHVYT

SEQ ID NO: 349 - protein, 171 aa, synthetic construct
IIDTYDEDEDEDSIKLSSIGSITPEMCKNLKQLVASKLKDIRQKEKSLRDYFTNLDDELDYMLLQGVGENHKKKCGCYILHLLLKFYSKTIRNTIQSEKHKNVNLELTNIAVSMLALEDLLEKCGITCNPKKDPLLKRIEDYMKQHGDDGVNKAIAELEFLFQMIEKQVYI

SEQ ID NO: 350 - protein, 146 aa, synthetic construct
AAQCRKGTITSRLKMLRTAFEKVREFYEDRDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 351 - protein, 151 aa, synthetic construct
MYVQHGSDYCTTTVHADIASAISGMRAEYDSGLGHYFKSLVPHPDNPYDTDDYKYMINNTNSYNCHALQSTINALLGMYGYVDIDEPHQLAMMKLATHTMQAAMLLNKCAKQLGCYHIPFDVETLHEAHPDDVMASLDTALNLMSMVTNEI

SEQ ID NO: 352 - protein, 146 aa, synthetic construct
AAQCRKGTITSRLKMLRTAFEKVREFYEDSDEEETALASTEHLHGPESCSVIDELITHYTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 353 - protein, 149 aa, synthetic construct
ATIKCVGMSTLFNPELIQLRRLFGDGIKDFFQNKDEDLDNAFLNEDVQRELASDCGCDHLMDMLSLYVNDTIPKGMKTEDAPSGLGQMGQLMSSLYRKMDMCWSELGCSHNTRLTLQEYADKKGGWDNKALGESDILFDALELFFSKIK

SEQ ID NO: 354 - protein, 151 aa, synthetic construct
MYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPNDVMASLDTALNLMSMVTNEI

SEQ ID NO: 355 - protein, 151 aa, synthetic construct
MYARRSGDYCTTTVRADIASAISGMRAEYNSGLRDYFKSLVPHPDNPYDTDDYKYMLNNTNSYNCHALQSTINALLGMYGYVDIDESHQLAMMKLATHTMQTAMMLNKCAAQLGCYHIPFDLETLHEAHPDDVMASLDTALNLMSMITNEI

SEQ ID NO: 356 - protein, 151 aa, synthetic construct
MYVQHGSDYCTTTVRADIASAISGMRAEYNNGLGDYFKSLAPHPNNPYDTDDYKYMINSTNSYNCHALQSTINALLGMYGYVDIDEPHQLAMMKLATHTMQTAMLLNKCAAQLGCYHIPFDVETLHEAHPDDVMASLDTALNLMSMVTNEI

SEQ ID NO: 357 - protein, 150 aa, synthetic construct
NVHSGTEDNPCTNSKTVLNTLLNQIKQEYINNLLPYYKALTPKPVDVFDDSYTYSIQSTDYNCYTIYETLNFLLGDVFPRATTDATVRLSLAKIATSSQQASMLMNLCKKELACGPAPFDMIKLYHDTKEYGADNIMGTLDTPFQYFVIV

SEQ ID NO: 358 - protein, 152 aa, synthetic construct
MYVQHGRGDYCTTTVRADIASAISGMRAEYDSGLGHYFKSLVPHPDNPYDTDDYKYMINNTNSYNCHALQSTINALLGMYGYVDIDEPHQLAMMKLATHTMQTAMLLNKCAEQLGCYHIPFDVEILHEAHPDDVMASLDTALNLMSMVTNEI

SEQ ID NO: 359 - protein, 162 aa, synthetic construct
APATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCRDFLKCEDLPHWHQMAENYKEKPMQGFSEMDFVFQSVEKFLVAKDVKNMKTKRKH

SEQ ID NO: 360 - protein, 152 aa, synthetic construct
DDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCRQQLGCGPPPFDAKTLYDDRAEYGADDIMATLDTALAKFKLVLESENVV

SEQ ID NO: 361 - protein, 152 aa, synthetic construct
DDDPCTNVKTQLNTLFNQIKTEYDTNLKTYYQSIAPSAFDPFNNTNYLYSVQGNDYKCYTIFETLSFLMGDVYPRATTNESVRLSLAKVATSSTQGAMVMNLCREQLGCGPPPFDAKTLYDDRAEYGADDIMATLDTALAKFKLVLESENVV

SEQ ID NO: 362 - protein, 148 aa, synthetic construct
RRRGDYCTTTVRADIASAISGMRAEYNSGLGDYFKSLVPHPDNPYDTDDYKHMIDNANSYNCHALQSTINALLGMYGYVDIDEPHQLAMMKLATHTMQTAMLLNKCAAQLGCYHIPFDLETLREAPPADVMASLDTALNLMSMITNEI

SEQ ID NO: 363 - protein, 162 aa, synthetic construct
ASLSTHYNNYDLTRIATIDKDVCKRVAQHINDDFVNMRKLYETQLKNYFQQLVPNPTDVFKDDSYMYMINGTDYNCHIIYETMRFLSGDVFPFATETEAELQYMWKMMLGVSQLSAYIGNCYQYFKCGPAPFDPQVLYHDRELFHADTVMAYLDTAFSHFTL

SEQ ID NO: 364 - protein, 138 aa, synthetic construct
LNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNTVNVLEIKKLLLIDNNCKKLFSEIDIFLTWVMAKI

SEQ ID NO: 365 - protein, 138 aa, synthetic construct
LNCGIEHNELNNIKNIFFKVRNVVQADDVDHNLRILTPALLNNITVSETCFFIYDMFELYLNDVFVKYTNTALKLNILKSLSSVANNFLAIFNKVKKRRVKKNNVNVLEIKKLLLIDNNCKKLFSEIDIFLTWVMAKI

SEQ ID NO: 366 - protein, 117 aa, synthetic construct
APATTPKDSCVYLIGQTPQLLRQLRNAYQAIIGADGSGVDEDDMPIYPSDVMNELASTSVACDAIKKVLTMNIGILPNVTAAYPDKKSEVDEIGDNLSRLHQNIVNCVSRTQHLCYD

SEQ ID NO: 367 - protein, 161 aa, synthetic construct
DNKYDSESGDDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 368 - protein, 161 aa, synthetic construct
DNKYDSESGNDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 369 - protein, 161 aa, synthetic construct
DNKYDSESGNDCPTLPTSLPHMLHELRAAFSRVKTFFQMKDQLDNMLLDGSLLEDFKGYLGCQALSEMIQFYLEEVMPQAENHSTDQEKDKVNSLGEKLKTLRVRLRRCHRFLPCENKSKAVEQVKSAFSKLQEKGVYKAMSEFDIFINYIEAYMTTKMKN

SEQ ID NO: 370 - protein, 161 aa, synthetic construct
DNKYDSESGDDCPTLPTSLPHMLHELRAAFSRVKTFFQMKDQLDNMLLDGSLLEDFKGYLGCQALSEMIQFYLEEVMPQAENHSTGQEKDKVNSLGEKLKTLRVRLRRCHRFLPCENKSKAVEQVKSAFSKLQEKGVYKAMSEFDIFINYIEAYMTTKMKN

SEQ ID NO: 371 - protein, 161 aa, synthetic construct
DNRYDGQDGNDCPTLPTSLPHMLHELRAAFSRVKTFFQMKDQLDNMLLDGSLLEDFKGYLGCQALSEMIQFYLEEVMPQAENHSPDQDKNKVNSLGEKLKTLRVRLRRCHRFLPCENKSKAVEQVKSAFSKLQEKGVYKAMSEFDIFINYIEAYMTTKMKN

SEQ ID NO: 372 - protein, 150 aa, synthetic construct
ATTAIKNTKPQCRPEDYATRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 373 - protein, 151 aa, synthetic construct
ATTTTIKNTKPQCRPEDYATRLQDLRVTFDRVKPTLQREDDYSVWLDGTVVKGCWGCSVMDWLLRRYLEIVFPAGDHVYPGLKTELHSMRSTLESIYKDMRQCPLLGCGDKSVISRLSQEAEKKSDNGTRKGLSELDTLFSRLEEYLHSRK

SEQ ID NO: 374 - protein, 152 aa, synthetic construct
ATTTTTIKNTKPQCRPEDYATRLQDLRVTFYRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 375 - protein, 149 aa, synthetic construct
ATTTTTIKNTKPQCRPEDYATRLQDLRVTFHRVKPTLDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 376 - protein, 145 aa, synthetic construct
STKKCDDVSFDYILKDLRSEFSKIKSFVQDNDQENMMLLSQSMLDKLTSRIGCKSLSDMIKFYLNDVLPNAEKIEHMKNKITSIGEKLKSLKEKLISCDFLHCENHDEIKTVKTIFNKLKDKGIYKAMGEFDIFINYLEKYIVKK

SEQ ID NO: 377 - protein, 123 aa, synthetic construct
ATTTIKNTKPQCRPEDYATRLQDLRVTFHRVKPTLQREDDYSVWLDGDHVYPGLKTELHSMRSTLESIYKDMRQCPLLGCGDKSVISRLSQEAERKSDNGTRKGLSELDTLFSRLEEYLHSRK

SEQ ID NO: 378 - protein, 170 aa, synthetic construct
MIGTCYDEDEEIERLKSNSISSITPGMCRNLKHSVMIRLIDARQIEASIRSYFTDGDNNLSFMLLQGIREISKKKCGCYILNLMLRFYIQTIKHTILSNKHKDMNLELTNLAVTILSLESLLEKCGVTCNPVKDPLLTRIEEYTRKHGDNAIYKTIGELEFLFDAIEKFV

SEQ ID NO: 379 - protein, 144 aa, synthetic construct
QCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVIGM

SEQ ID NO: 380 - protein, 144 aa, synthetic construct
QCRKGTITIRLKMLRTAFEKVREFYEDRDEEETALASTEHLHGPESCSVIDELITHYTKCVIPAANEEEGADLRSLDTLQFALENVKGLLANCQEEFGCKPPFSMRDYKKQYRQLNKEKNAGMIKAMGELGMLFNGIEERVNGM

SEQ ID NO: 381 - protein, 144 aa, synthetic construct
QCRKGTITIRLKMLRTAFEKVREFYEDRDEEETALASTEHLHGPESCSVIDELITHYTKCVIPAANEEEGADLLSLDTLQFALENVKGLLANCQEEFGCKPPFSMRDYKKQYRQLNKEKNAGMIKAMGELGMLFNGIEERVIGM

SEQ ID NO: 382 - protein, 146 aa, synthetic construct
AAQCRKGTITSRLKMLRTAFEKVREFYEDRDEEETALASTEHLHGPESCSVIDELITHHTKCVIPAANEEEGADLLSLDTLQVALENVKGLLANCQEEFGCKPPFSMRDYKKQYRQLNKEKNAGMIKAMGELGMLFNGIEERVIGM

SEQ ID NO: 383 - protein, 100 aa, synthetic construct
ATTTIKNTKPQCRPEDYATRLQDLRVTFHRVKPTLVGHVGDHVYPGLKTELHSMRSTLESIYKDMRQCEAERKSDNGTRKGLSELDTLFSRLEEYLHSRK

SEQ ID NO: 384 - protein, 160 aa, synthetic construct; identical to SEQ ID NO: 2

SEQ ID NO: 385 - protein, 160 aa, synthetic construct
SPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 386 - protein, 160 aa, synthetic construct
SPGQGTQSENSCTHFPGNLPNMLRALRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 387 - protein, 95 aa, synthetic construct
TVYVVKSGVNKGKICDTCPPGTYKKRDCDRSLPTVCEPCGDGEYTSMNNSLPECLSCNHCYDPTEIEITPCNATTNTVCSCKEGYTFDSSIQGCI

SEQ ID NO: 388 - protein, 91 aa, synthetic construct
STYRVRSSGLTCSTCPPGTHKERDCSLNTETICKACGEGEYTAHNNSLPKCLACKSCFNATEIETKSCDPTSDTICACREGYSINNLGECN

SEQ ID NO: 389 - protein, 91 aa, synthetic construct
STYRVRSSGLTCSMCPPGTYKERDCSLNTETLCKACGEGEYTAHKNSLPKCLACKSCFNATEIETKSCDPISDTICTCREGYSINNLGECN

SEQ ID NO: 390 - protein, 90 aa, synthetic construct
RSKINSSLICDMCPPGYYKNKDCTSTSTTICLPCGEGEYTAYNNSLTKCIRCKDCYEENEKIFKPCNSTSDTICTCIDGYTKDETTDSCI

SEQ ID NO: 391 - protein, 91 aa, synthetic construct
DTFTDEETGLVCEKCPPGTYREADCTETTKTVCKACGEGTFTDHPNNLPECKSCSTCDEATEVEVQKCTATANTVCACKDGFTVNEKGECV

SEQ ID NO: 392 - protein, 91 aa, synthetic construct
DTYTDEKTGLTCEKCPPGTYREADCGENSKTVCKACGEGTYTDAPNHLPECKKCNTCDAETEVEVKACTATSDTVCACKEGFTVNENGECV

SEQ ID NO: 393 - protein, 91 aa, synthetic construct
STYTDSATGRTCSKCPPGTYRSADCGATTSTVCTACGAGTYTDKDNNLSACLACSTCNAATETEVSACTATANTVCACKAGYTLDANGKCV

SEQ ID NO: 394 - protein, 91 aa, synthetic construct
STYKDEKTGLTCQKCPPGTYRKADCGLFSKTECAACGEGTYTDKDNNLSKCLKCSTCNEETEVEVQKCTATADTVCACKEGFTKNEKGECQ

SEQ ID NO: 395 - protein, 91 aa, synthetic construct
PTYRDEATGRVCQECPPGTYLEAHCGENTSTVCKACGEGTFTDKPNHLPQCLSCSTCEAATEVEVRACTATANTVCACREGFTRNEQGKCN

SEQ ID NO: 396 - protein, 91 aa, synthetic construct
DTFTDEATGLTCAKCPPGTYREADCGLFSKTVCKACGEGTFTAEPNNLPTCKACNTCDAATEVEVQACTATADTVCACKEGFTKNENGECN

SEQ ID NO: 397 - protein, 91 aa, synthetic construct
ATYTDTATGLTCAKCPPGTFRKADCGATTATVCEACGAGTYTAADNHLSACLACTTCDAATETETAACTATANTVCACKAGFTVDANGKCG

SEQ ID NO: 398 - protein, 91 aa, synthetic construct
PTYTDTATGLTCAKCPPGTYRERDCGLFSDTVCKACGEGTYTDTPNHLPKCLSCSTCDAATETEVQACTATANTVCACKDGFTVDSNGKCQ

SEQ ID NO: 399 - protein, 91 aa, synthetic construct
DTYTDTATGLTCAKCPPGTYREADCGLFSETVCKACGAGTYTDKPNHLPACLACGTCDAATEVEVQACTATANTVCACKAGFTKNEKGECV

SEQ ID NO: 400 - protein, 91 aa, synthetic construct
PTYTDTATGLTCEQCPPGTYRRADCGATTATVCAACGAGTYTSAPNNLPTCLACPTCDAATEVEVQPCVATANTVCACRAGFTVDAQGRCQ

SEQ ID NO: 401 - protein, 91 aa, synthetic construct
ATYTDTATGLTCTKCPPGTHRKADCGLLTSTVCEACGEGTYTSEDNNLSSCLSCSTCNEATETEVQACTATSDTVCECKEGFTVDANGKCG

SEQ ID NO: 402 - protein, 91 aa, synthetic construct
ATYKDEKTGLTCEKCPPGTYREADCTETTKTVCKACGEGTYTDKDNNLPKCLECNTCDEKTEVEVQKCTATSNTVCACKEGFTKNEKGECK

SEQ ID NO: 403 - protein, 91 aa, synthetic construct
KTYKDPETGLTCEKCPPGTYLEANCTPTSPTVCKPCGEGTYTEEENALTACKPCSSCDPATEEVVQPCTPTSDTVCSCKPGYSFNSNGLCV

SEQ ID NO: 404 - protein, 91 aa, synthetic construct
ETYTDSSTGLTCAKCPPGYYLSQTCTETSPTVCKACGSGTYTTTPNALTSCKACSTCDSSTEVTTKSCTPTSDTVCSCKSGYSKNSSGKCV

SEQ ID NO: 405 - protein, 91 aa, synthetic construct
QTYTDPATGLTCDLCPPGTYLEAACTATSPTVCKPCPSGTYTTAPNALTSCRKCSTCNPATEVTTQPCTPTSDTVCSCKSGYTLDSNGQCV

SEQ ID NO: 406 - protein, 91 aa, synthetic construct
KTYVDPTTGLTCDMCPPGTYLKKTCSLNSRTVCKPCGKGTYTSTYNNLKKCKKCSTCDPRTEVVSQPCTQTSDTVCECRPGYSRDSNGRCV

SEQ ID NO: 407 - protein, 91 aa, synthetic construct
ATYTDPETGLTCEKCPPGTYLEAPCTETTPTVCKPCGEGEYTTVPNALRKCRECTTCNPATEEVSADCTPTSNTVCTCKPGYVKDANGKCV

SEQ ID NO: 408 - protein, 91 aa, synthetic construct
QTYVDPATGLTCTLCPPGTYLKKPCTATSPTVCKPCGSGTYTSKPNALTKCQECTTCDPATERVVRPCTPTHNTVCECKPGYKRNSKGQCV

SEQ ID NO: 409 - protein, 91 aa, synthetic construct
ATYTDPATGLVCELCPPGSYLAAPCTATTPTVCKPCPEGYYTTEPNALPRCLPCTTCNPETEVVIEPCTPTSDTVCECKPGYSLNENGECV

SEQ ID NO: 410 - protein, 91 aa, synthetic construct
DTYTDPTTGLTCKKCPPGTYLKKNCTATSPTVCKKCGPGQYTTKYNNLKKCNKCSTCDPTTEEVSQACTPTHDTVCKCKPGYSLNSKGRCV

SEQ ID NO: 411 - protein, 91 aa, synthetic construct
STYTNPSTGLTCSMCPPGTYLERDCSSSSGTVCKPCGPGTYTTAYNNLKSCKKCTKCDPKTEKVVKECTPTSNTVCECKPGFKFDSNGKCR

SEQ ID NO: 412 - protein, 91 aa, synthetic construct
KTFVDDKTGLTCEQCPPGTYLESTCSETSPTVCKPCGPGEYTTEYNNLTKCKKCSTCDPATEVVVSACTPTSDTVCACKPGFSFNEEGKCV

SEQ ID NO: 413 - protein, 91 aa, synthetic construct
STYVNPTTGLTCSKCPPGTYLKASCTATSKTVCTPCGSGTYTTTANNLSSCKQCSTCDPATETVSQPCTPTSDTVCSCKSGYTFDSNGKCV

SEQ ID NO: 414 - protein, 91 aa, synthetic construct
ETYKDPSTGLLCEKCPPGTYLSSPCTTTSGTVCKPCGSGTYTTQPNALSSCSSCSTCDSATQTVSKACTSTSDTVCSCKSGYSLDSNGNCV

SEQ ID NO: 415 - protein, 91 aa, synthetic construct
ETYTDPATGRVCEKCPPGTYVAAPCTATSPTVCRPCGPGTYTSAPNALTSCLACTSCDPATEAVSQPCTPTANTVCVCKPGFSFNANGQCV

SEQ ID NO: 416 - protein, 91 aa, synthetic construct
ETYTNASTGLTCNKCPPGTYLAKDCTSTSDTVCQDCGPGTYTTSPNAKSSCNACSTCDPATEEVITPCTPTSDTVCACKPGFTWNQNGQCV

SEQ ID NO: 417 - protein, 91 aa, synthetic construct
ETYTDPATGLTCQKCPPGTYLKAPCTSTSPTVCTPCGSGYYTSSSNNLTSCSKCNTCDPSTESVSSACTPTSNTVCSCKSGYTLDSNGNCV

SEQ ID NO: 418 - protein, 95 aa, synthetic construct
MSTYNTKTSPSLKCDMCPPGYYKHEDCTSNTKTVCSPCGEGEYTAYNNSLTKCLRCSDCYGENEITTKQCTNTSNTVCECMDGYTKDETIDACIK

SEQ ID NO: 419 - protein, 100 aa, synthetic construct
ASTYTSKIDASLICDMCPPGSYKYKDCTYDSKTVCLPCGDGEYTSYNNSLAKCLRCDDCYDENEVTAKPCDSTSNTICKCMDGYTKDNTISACVKTSHIT

SEQ ID NO: 420 - protein, 258 aa, synthetic construct
KVPTWTDPVTGLTCDRCPAGTHVVKQCTATTPTECGACPANHYTEFWNYLNRCLYCGVRCTEQQVEKSTCSATHNRVCECKPGYHIYADDFCLRHSTCPPGQGLLVAGTPTSNTRCGPCQAGYFSAEDSTEACKPHTPCKDTERSVPGTATQDTFCTSCQARKCDITAPPAPDKAVCDDAFIDFVERYPLNAKKAKNLMRKLRKMGKGKTIRESFQAIMAKRNDQPLACEVLSTLNDVDSDMGCRIKTFFLGWNEDEC

SEQ ID NO: 421 - protein, 112 aa, synthetic construct
MIITLLALLLVVSTESSTVYVVKSGVNKGKICDTCPPGTYKKRDCDRSLPTVCEPCGDGEYTSMNNSLPECLSCNHCYDPTEIEITPCNATTNTVCSCKEGYTFDSSIQGCI

SEQ ID NO: 422 - protein, 116 aa, synthetic construct
MGKNSLVLTIGVLMTLITLRSSFSASTYRVRSSGLTCSTCPPGTHKERDCSLNTETICKACGEGEYTAHNNSLPKCLACKSCFNATEIETKSCDPTSDTICACREGYSINNLGECN

SEQ ID NO: 423 - protein, 116 aa, synthetic construct
MGKNSLVLMIGVLMTLITLRSSFSASTYRVRSSGLTCSMCPPGTYKERDCSLNTETLCKACGEGEYTAHKNSLPKCLACKSCFNATEIETKSCDPISDTICTCREGYSINNLGECN

SEQ ID NO: 424 - protein, 117 aa, synthetic construct
MHYKNKMSVNILIITVLIGISFQASTYRSKINSSLICDMCPPGYYKNKDCTSTSTTICLPCGEGEYTAYNNSLTKCIRCKDCYEENEKIFKPCNSTSDTICTCIDGYTKDETTDSCI

SEQ ID NO: 425 - protein, 116 aa, synthetic construct
MGKNSLVLTIGVLMTLITLRSSFSADTFTDEETGLVCEKCPPGTYREADCTETTKTVCKACGEGTFTDHPNNLPECKSCSTCDEATEVEVQKCTATANTVCACKDGFTVNEKGECV

SEQ ID NO: 426 - protein, 116 aa, synthetic construct
MGKNSLVLTIGVLMTLITLRSSFSADTYTDEKTGLTCEKCPPGTYREADCGENSKTVCKACGEGTYTDAPNHLPECKKCNTCDAETEVEVKACTATSDTVCACKEGFTVNENGECV

SEQ ID NO: 427 - protein, 116 aa, synthetic construct
MGKNSLVLTIGVLMTLITLRSSFSASTYTDSATGRTCSKCPPGTYRSADCGATTSTVCTACGAGTYTDKDNNLSACLACSTCNAATETEVSACTATANTVCACKAGYTLDANGKCV

SEQ ID NO: 428 - protein, 116 aa, synthetic construct
MGKNSLVLTIGVLMTLITLRSSFSASTYKDEKTGLTCQKCPPGTYRKADCGLFSKTECAACGEGTYTDKDNNLSKCLKCSTCNEETEVEVQKCTATADTVCACKEGFTKNEKGECQ

SEQ ID NO: 429 - protein, 116 aa, synthetic construct
MGKNSLVLTIGVLMTLITLRSSFSAPTYRDEATGRVCQECPPGTYLEAHCGENTSTVCKACGEGTFTDKPNHLPQCLSCSTCEAATEVEVRACTATANTVCACREGFTRNEQGKCN

SEQ ID NO: 430 - protein, 116 aa, synthetic construct
MGKNSLVLTIGVLMTLITLRSSFSADTFTDEATGLTCAKCPPGTYREADCGLFSKTVCKACGEGTFTAEPNNLPTCKACNTCDAATEVEVQACTATADTVCACKEGFTKNENGECN

SEQ ID NO: 431 - protein, 116 aa, synthetic construct
MGKNSLVLTIGVLMTLITLRSSFSAATYTDTATGLTCAKCPPGTFRKADCGATTATVCEACGAGTYTAADNHLSACLACTTCDAATETETAACTATANTVCACKAGFTVDANGKCG

SEQ ID NO: 432 - protein, 116 aa, synthetic construct
MGKNSLVLTIGVLMTLITLRSSFSAPTYTDTATGLTCAKCPPGTYRERDCGLFSDTVCKACGEGTYTDTPNHLPKCLSCSTCDAATETEVQACTATANTVCACKDGFTVDSNGKCQ

SEQ ID NO: 433 - protein, 116 aa, synthetic construct
MGKNSLVLTIGVLMTLITLRSSFSADTYTDTATGLTCAKCPPGTYREADCGLFSETVCKACGAGTYTDKPNHLPACLACGTCDAATEVEVQACTATANTVCACKAGFTKNEKGECV

SEQ ID NO: 434 - protein, 116 aa, synthetic construct
MGKNSLVLTIGVLMTLITLRSSFSAPTYTDTATGLTCEQCPPGTYRRADCGATTATVCAACGAGTYTSAPNNLPTCLACPTCDAATEVEVQPCVATANTVCACRAGFTVDAQGRCQ

SEQ ID NO: 435 - protein, 116 aa, synthetic construct
MGKNSLVLTIGVLMTLITLRSSFSAATYTDTATGLTCTKCPPGTHRKADCGLLTSTVCEACGEGTYTSEDNNLSSCLSCSTCNEATETEVQACTATSDTVCECKEGFTVDANGKCG

SEQ ID NO: 436 - protein, 116 aa, synthetic construct
MGKNSLVLTIGVLMTLITLRSSFSAATYKDEKTGLTCEKCPPGTYREADCTETTKTVCKACGEGTYTDKDNNLPKCLECNTCDEKTEVEVQKCTATSNTVCACKEGFTKNEKGECK

SEQ ID NO: 437 - protein, 116 aa, synthetic construct
MGKNSLVLTIGVLMTLITLRSSFSAKTYKDPETGLTCEKCPPGTYLEANCTPTSPTVCKPCGEGTYTEEENALTACKPCSSCDPATEEVVQPCTPTSDTVCSCKPGYSFNSNGLCV

SEQ ID NO: 438 - protein, 116 aa, synthetic construct
MGKNSLVLTIGVLMTLITLRSSFSAETYTDSSTGLTCAKCPPGYYLSQTCTETSPTVCKACGSGTYTTTPNALTSCKACSTCDSSTEVTTKSCTPTSDTVCSCKSGYSKNSSGKCV

SEQ ID NO: 439 - protein, 116 aa, synthetic construct
MGKNSLVLTIGVLMTLITLRSSFSAQTYTDPATGLTCDLCPPGTYLEAACTATSPTVCKPCPSGTYTTAPNALTSCRKCSTCNPATEVTTQPCTPTSDTVCSCKSGYTLDSNGQCV

SEQ ID NO: 440 - protein, 116 aa, synthetic construct
MGKNSLVLTIGVLMTLITLRSSFSAKTYVDPTTGLTCDMCPPGTYLKKTCSLNSRTVCKPCGKGTYTSTYNNLKKCKKCSTCDPRTEVVSQPCTQTSDTVCECRPGYSRDSNGRCV

SEQ ID NO: 441 - protein, 116 aa, synthetic construct
MGKNSLVLTIGVLMTLITLRSSFSAATYTDPETGLTCEKCPPGTYLEAPCTETTPTVCKPCGEGEYTTVPNALRKCRECTTCNPATEEVSADCTPTSNTVCTCKPGYVKDANGKCV

SEQ ID NO: 442 - protein, 116 aa, synthetic construct
MGKNSLVLTIGVLMTLITLRSSFSAQTYVDPATGLTCTLCPPGTYLKKPCTATSPTVCKPCGSGTYTSKPNALTKCQECTTCDPATERVVRPCTPTHNTVCECKPGYKRNSKGQCV

SEQ ID NO: 443 - protein, 116 aa, synthetic construct
MGKNSLVLTIGVLMTLITLRSSFSAATYTDPATGLVCELCPPGSYLAAPCTATTPTVCKPCPEGYYTTEPNALPRCLPCTTCNPETEVVIEPCTPTSDTVCECKPGYSLNENGECV

SEQ ID NO: 444 - protein, 116 aa, synthetic construct
MGKNSLVLTIGVLMTLITLRSSFSADTYTDPTTGLTCKKCPPGTYLKKNCTATSPTVCKKCGPGQYTTKYNNLKKCNKCSTCDPTTEEVSQACTPTHDTVCKCKPGYSLNSKGRCV

SEQ ID NO: 445 - protein, 116 aa, synthetic construct
MGKNSLVLTIGVLMTLITLRSSFSASTYTNPSTGLTCSMCPPGTYLERDCSSSSGTVCKPCGPGTYTTAYNNLKSCKKCTKCDPKTEKVVKECTPTSNTVCECKPGFKFDSNGKCR

SEQ ID NO: 446 - protein, 116 aa, synthetic construct
MGKNSLVLTIGVLMTLITLRSSFSAKTFVDDKTGLTCEQCPPGTYLESTCSETSPTVCKPCGPGEYTTEYNNLTKCKKCSTCDPATEVVVSACTPTSDTVCACKPGFSFNEEGKCV

SEQ ID NO: 447 - protein, 116 aa, synthetic construct
MGKNSLVLTIGVLMTLITLRSSFSASTYVNPTTGLTCSKCPPGTYLKASCTATSKTVCTPCGSGTYTTTANNLSSCKQCSTCDPATETVSQPCTPTSDTVCSCKSGYTFDSNGKCV

SEQ ID NO: 448 - protein, 116 aa, synthetic construct
MGKNSLVLTIGVLMTLITLRSSFSAETYKDPSTGLLCEKCPPGTYLSSPCTTTSGTVCKPCGSGTYTTQPNALSSCSSCSTCDSATQTVSKACTSTSDTVCSCKSGYSLDSNGNCV

SEQ ID NO: 449 - protein, 116 aa, synthetic construct
MGKNSLVLTIGVLMTLITLRSSFSAETYTDPATGRVCEKCPPGTYVAAPCTATSPTVCRPCGPGTYTSAPNALTSCLACTSCDPATEAVSQPCTPTANTVCVCKPGFSFNANGQCV

SEQ ID NO: 450 - protein, 116 aa, synthetic construct
MGKNSLVLTIGVLMTLITLRSSFSAETYTNASTGLTCNKCPPGTYLAKDCTSTSDTVCQDCGPGTYTTSPNAKSSCNACSTCDPATEEVITPCTPTSDTVCACKPGFTWNQNGQCV

SEQ ID NO: 451 - protein, 116 aa, synthetic construct
MGKNSLVLTIGVLMTLITLRSSFSAETYTDPATGLTCQKCPPGTYLKAPCTSTSPTVCTPCGSGYYTSSSNNLTSCSKCNTCDPSTESVSSACTPTSNTVCSCKSGYTLDSNGNCV

SEQ ID NO: 452 - protein, 117 aa, synthetic construct
MSNNLCQIITICIITMVCISYQMSTYNTKTSPSLKCDMCPPGYYKHEDCTSNTKTVCSPCGEGEYTAYNNSLTKCLRCSDCYGENEITTKQCTNTSNTVCECMDGYTKDETIDACIK

SEQ ID NO: 453 - protein, 123 aa, synthetic construct
MTNYLYISANIFVIILLAEITFQASTYTSKIDASLICDMCPPGSYKYKDCTYDSKTVCLPCGDGEYTSYNNSLAKCLRCDDCYDENEVTAKPCDSTSNTICKCMDGYTKDNTISACVKTSHIT

SEQ ID NO: 454 - protein, 276 aa, synthetic construct
MFRGTLMVLACVAACFSEKVPTWTDPVTGLTCDRCPAGTHVVKQCTATTPTECGACPANHYTEFWNYLNRCLYCGVRCTEQQVEKSTCSATHNRVCECKPGYHIYADDFCLRHSTCPPGQGLLVAGTPTSNTRCGPCQAGYFSAEDSTEACKPHTPCKDTERSVPGTATQDTFCTSCQARKCDITAPPAPDKAVCDDAFIDFVERYPLNAKKAKNLMRKLRKMGKGKTIRESFQAIMAKRNDQPLACEVLSTLNDVDSDMGCRIKTFFLGWNEDEC

SEQ ID NO: 455 - protein, 103 aa, synthetic construct
DSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 456 - protein, 108 aa, synthetic construct
SLQCKNNTYYNSQYVKCCKLCEPGTFYSKKCDEKNDTICEKCPDGSYTSVYNHSPACVSCRGYCDYNQVETTSCTPTSNRICKCKLSSYCLVKGYNENCRVCVRKKMN

SEQ ID NO: 457 - protein, 102 aa, synthetic construct
DIAPHAPSDGKCKDNEYKRHNLCPGTYASRLCDSKTNTQCTPCGSGTFTSRNNHLPACLSCNGRRDRVTRLTIESVNALPDIIVFSKDHPDARHVFPKQNVE

SEQ ID NO: 458 - protein, 99 aa, synthetic construct
DIAPHAPSDGKCKDNEYKRHNLCPGTYASDSKTNTRCTPCGSGTFTSRNNHLPACLSCNGRRDRVTRLTIESVNALPDIIVFSKDHPDARHVFPKQNVE

SEQ ID NO: 459 - protein, 103 aa, synthetic construct
DNNCGELEYYNKVHDVCCKLCPAGFYAKQLCTKDMDTVCNPCATETFLSIPNYTSKCLSCRGKCTKDQVEVRPCTITRNRTCKCKDGYICILKTDDNSCRVCV

SEQ ID NO: 460 - protein, 124 aa, synthetic construct
MYKYSNYILYLLILVTVARSSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 461 - protein, 127 aa, synthetic construct
MVKLIVFIIGLLINSTYSLSLQCKNNTYYNSQYVKCCKLCEPGTFYSKKCDEKNDTICEKCPDGSYTSVYNHSPACVSCRGYCDYNQVETTSCTPTSNRICKCKLSSYCLVKGYNENCRVCVRKKMN

SEQ ID NO: 462 - protein, 122 aa, synthetic construct
MKSVLYSYILFLSCIIINGRDIAPHAPSDGKCKDNEYKRHNLCPGTYASRLCDSKTNTQCTPCGSGTFTSRNNHLPACLSCNGRRDRVTRLTIESVNALPDIIVFSKDHPDARHVFPKQNVE

SEQ ID NO: 463 - protein, 119 aa, synthetic construct
MKSVLYSYILFLSCIIINGRDIAPHAPSDGKCKDNEYKRHNLCPGTYASDSKTNTRCTPCGSGTFTSRNNHLPACLSCNGRRDRVTRLTIESVNALPDIIVFSKDHPDARHVFPKQNVE

SEQ ID NO: 464 - protein, 124 aa, synthetic construct
MYKHCYYILYLFISLTVVCSSDNNCGELEYYNKVHDVCCKLCPAGFYAKQLCTKDMDTVCNPCATETFLSIPNYTSKCLSCRGKCTKDQVEVRPCTITRNRTCKCKDGYICILKTDDNSCRVCV

SEQ ID NO: 465 - protein, 88 aa, synthetic construct
KTCPADYYLEPEDGLCTACVTCLSNMVEIQPCGPDKPRKCQCGPGLKCTLPAVNSCARCTPDTTTKKVQKEQCCTTPDNTKLCYHKYS

SEQ ID NO: 466 - protein, 89 aa, synthetic construct
KTCPADYYLEPEDGLCTACVTCLSNMVEIQPCGPDKPRKCQCGPGLKCTVPAVNSCARCTPDTTTKKIEPIGQCCTTPDNTKLCYHKYS

SEQ ID NO: 467 - protein, 92 aa, synthetic construct
KTCPADYYLNPENGLCTACVTCLSNMVEIQPCGPDKPRKCECGSGFKCTLPAVNSCARCTPDTTTKKVQKEQKEQCCNTPDNTKLCYHKYSS

SEQ ID NO: 468 - protein, 89 aa, synthetic construct
KTCPNDYYLEPEDGLCTACVTCLSNMVEIQPCGPDKPRKCKCGPGLKCTVPAVNSCARCTPDTTTKKIEPIGQCCTTPDNTKLCYHKYS

SEQ ID NO: 469 - protein, 89 aa, synthetic construct
KTCPNDYYLEPEDGLCTACVTCLSNMVEIQPCGPDKPRKCQCGPGLKCTVPAVNSCARCTPDTTTKKIEPIGQCCTTPDNTKLCYHKYS

SEQ ID NO: 470 - protein, 88 aa, synthetic construct
KTCPADYYLEPEDGLCTACVTCLSNMVETQSCGPDKPRKCQCGPGLKCTVPAVNSCARCTPDTTTKKVQKEQCCTTPDNTKLCYHKYS

SEQ ID NO: 471 - protein, 86 aa, synthetic construct
TCPNDYYLEPEDGLCTACVTCLSNMVEIQPCGPDKPRKCQCGPGLKCTVPAVNSCARCTPDTTIKKIKPTDQCCTTPDNTKLCYHK

SEQ ID NO: 472 - protein, 89 aa, synthetic construct
TCPNDYYLEPEDGLCTACVTCLSNMVEIQPCGPDKPRKCQCGPGLKCTVPAVNSCARCTPDTTIKKIEPTDQCCTTPDNTKLCYHKYSP

SEQ ID NO: 473 - protein, 88 aa, synthetic construct
TCPADYYLEPEDGLCTACVTCLSNMVEIQPCGPDKPRKCQCGPGLKCTVPAVNSCARCTPDTTTKKVQKDQCCTTPDNTKLCYHKYSS

SEQ ID NO: 474 - protein, 97 aa, synthetic construct
TCPVDHYYDREDGGLCTACVTCLQNMVEIQPCGPDKPRKCQCAPGFKCTLPAVNSCARCTPDSTYKPPPKPATKPKSHEDNCCITTGNTKLCYTQLN

SEQ ID NO: 475 - protein, 83 aa, synthetic construct
DCEKDYYRDPKTGRCTACVTCTGDTVEKSPCGPTTPRKCECGPGLKCTVSATNTCARCEEHIPDNCCKTKDNTKLCYQKLRGS

SEQ ID NO: 476 - protein, 88 aa, synthetic construct
TCPADYYLEPEDGLCTACVTCLSNMVETQSCGPDKPRKCQCGPGLKCTVPAVNSCARCTPDTTTKKVQKEQCCTTPDNTKLCYHKYSS

SEQ ID NO: 477 - protein, 69 aa, synthetic construct
MKCEQCVSYYNTQELKCCKLSKPGTYSDHRCDKYSDTICGHCPSDTFTSIYNRSPRCHSCRGHTLYTYH

SEQ ID NO: 478 - protein, 69 aa, synthetic construct
MKCEQCVSYYNTQELKCCKLSKPGTYSDHRCDKYSDTICGHCPSDTFTSIYNRSPRCYSCRGHTLYTYH

SEQ ID NO: 479 - protein, 77 aa, synthetic construct
TCTEGSFLNSLDKKCYDCPYNTWQASRRHSEIVCNMCTQCKPGYTLTQKCTPTTDTVCMCRKPFVEKNGFCMLFHKP

SEQ ID NO: 480 - protein, 78 aa, synthetic construct
YYKDPTTNRCTACVTCGGDMVETAPCGPNTPRKCECGPGLKCALAVQNSCARCEEYIPDNCCKTKDKTKLCYEKLRGS

SEQ ID NO: 481 - protein, 88 aa, synthetic construct
TCPNDYYLEPEDGLCKACVTCLSNMVEIQPCGPDKPRKCQCGPGLKCTVPVVNSCARCTPDTTTKKVQKEQCCTTPDNTKLCYHKYSS

SEQ ID NO: 482 - protein, 88 aa, synthetic construct
TCPADYYLEPEDGLCTACVTCLSNMVEIQPCGPDKPRKCQCGPGLKCTVPAVNSCARCTPDTTTKKVQKEQCCTTPDNTKLCYHKYSS

SEQ ID NO: 483 - protein, 88 aa, synthetic construct
MVCCRLYPAGSYAEQLCTKDNDTVCNPCATETFLSIPNYTSKCLSCRGKCTKDQVEVRPCTITRNRTCKCKDGYICILKTDDNSCRVC

SEQ ID NO: 484 - protein, 89 aa, synthetic construct
TCPNDYYLEPEDGLCTACVTCLSNMVEIQPCGPNKPRKCQCGPGLKCTVPAVNSCARCTPDTTIKKIEPTDQCCTTPDNTKLCYHKYSS

SEQ ID NO: 485 - protein, 89 aa, synthetic construct
TCPNDYYLEPEDGLCTACVTCLSNMVEIQPCGPDKPRKCQCGPGLKCTVPAVNSCARCTPDTTIKKIEPTDQCCTTPDNTKLCYHKYSS

SEQ ID NO: 486 - protein, 89 aa, synthetic construct
TCPNDYYLEPEDGLCKACVTCLSNMVEIQSCGPDKPRKCQCGPGLKCMLPAVNSCARCTPDTTTKKIEPTEQCCTTPDNTKLCYHKYSS

SEQ ID NO: 487 - protein, 89 aa, synthetic construct
TCPADYYLEPEDGLCTACVTCLSNMVETQPCGPDKPRKCQCGPGLKCTLPAVNSCARCTPDTTTKKVQKEQCCTTPDNTKLCYHKYSSS

SEQ ID NO: 488 - protein, 89 aa, synthetic construct
TCPNDYYLEPEDGLCTACVTCLSNMVEIQPCGPDKPRKCQCGPGLKCTVPAVNSCARCTPDTTTKKIEPIGQCCTTPDNTKLCYHKYSS

SEQ ID NO: 489 - protein, 89 aa, synthetic construct
TCPNDYYLEPEDGLCKACVTCLSNMVEIQSCGPDKPRKCQCGPGLKCTLPAVNSCARCTPDTTTKKIEPTEQCCTTPDNTKLCYHKYSS

SEQ ID NO: 490 - protein, 89 aa, synthetic construct
TCPADYYLEPEDGLCTACVTCLSNMVEIQPCGPDKPRKCQCGPGLKCTVPAVNSCARCTPDTTTKKIEPIGQCCTTPDNTKLCYHKYSS

SEQ ID NO: 491 - protein, 89 aa, synthetic construct
TCPNDYYLEPEDGLCTACVTCLSNMVEIQPCGPDKPRKCKCGPGLKCTVPAVNSCARCTPDTTTKKIEPIGQCCTTPDNTKLCYHKYSS

SEQ ID NO: 492 - protein, 89 aa, synthetic construct
YASRLCDSKTNTQCTPCGSGTFTSRNNHLPACLSCNGRCDSNQVETRSCNTTHNRICECSPGYYCILKGSSGCKACVSQNKCGIGYGVS

SEQ ID NO: 493 - protein, 91 aa, synthetic construct
TCPADYYLNPENGLCTACVTCLSNMVEIQPCGPDKPRKCECGSGFKCTLPAVNSCARCTPDTTTKKVQKEQKEQCCNTPDNTKLCYHKYSS

SEQ ID NO: 494 - protein, 96 aa, synthetic construct
KICQNDYYFNKETGHCTACVTCVGNTKETHPCGPDEPRTCECDSGFKCDLPVANSCARCIVIPPTKSTKNKHIKEQCCNTTDNVKLCYTIQNSFIK

SEQ ID NO: 495 - protein, 98 aa, synthetic construct
TCPTDYYYNREDDGLCTACVTCLKNMVEIQPCGPDKPRKCQCAPGFKCTVPAVNSCARCTPDSTYKPPIPKPASKPKSPDDNCCVTTSNIKLCYTQLN

SEQ ID NO: 496 - protein, 99 aa, synthetic construct
DIAPHAPSDGKCKDNEYKRHNLCPGTYASRLCDSKTNTQCTPCTFTSRNNHLPACLSCNGRRDRVTRLTIESVNALPDIIVFSKDHPDARHVFPKQNVE

SEQ ID NO: 497 - protein, 99 aa, synthetic construct; identical to SEQ ID NO: 458

SEQ ID NO: 498 - protein, 99 aa, synthetic construct
RDIAPHAPSDGKCKDNEYKRHNLCPGTYASRLCDSKTNTQCTPCTFTSRNNHLPACLSCNGRRDRVTRLTIESVNALPDIIVFSKDHPDARHVFPKQNV

SEQ ID NO: 499 - protein, 99 aa, synthetic construct
RDIAPHAPSDGKCKDNEYKRHNLCPGTYASRLCDSKTNTQCTPCTFTSRNNHLPACLSCNGRRDRVTLLTIESVNALPDIIVFSKDHPDARHVFPKQNV

SEQ ID NO: 500 - protein, 100 aa, synthetic construct
EVKLGNQCCPPCGSGQKVTKVCTENSGITCTLCPNGTYLTGLYNCTNCTQCNDTQITVRNCTSTNNTICASKNHTLFSTPGVQHHKQRQQNHTAHVTVKQ

SEQ ID NO: 501 - protein, 102 aa, synthetic construct
DIAPHAPSNGKCKDNEYKRHNLCPGTYASRLCDSKTNTRCTPCGSGTFTSRNNHLPACLSCNGRRDRVTLLTIESVNALPDIIVFSKDHPDARHVFPKQNVE

SEQ ID NO: 502 - protein, 102 aa, synthetic construct; identical to SEQ ID NO: 457

SEQ ID NO: 503 - protein, 102 aa, synthetic construct
DIAPHAPSDGKCKDNEYKRHNLCPGTYASRLCDSKTNTRCTPCGSGTFTSRNNHLPACLSCNGRRDRVTRLTIESVNALPDIIVFSKDHPDARHVFPKQNVE

SEQ ID NO: 504 - protein, 102 aa, synthetic construct
RDIAPHAPSDGKCKDNEYKRHNLCPGTYASRLCDSKTNTQCTPCGSGTFTSRNNHLPACLSCNGRRDRVTRLTIESVNALPDIIVFSKDHPDARHVFPKQNV

SEQ ID NO: 505 - protein, 102 aa, synthetic construct
RDIAPHAPSDGKCKDNEYKRHNLCPGTYASRLCDSKTNTQCTPCGSGTFTSRNNHLPACLSCNGRCDRVTLLTIESVNALPDIIVFSKDHPDARHVFPKQNV

SEQ ID NO: 506 - protein, 102 aa, synthetic construct
RDIAPHAPSNGKCKDNEYKRHNLCPGTYASRLCDSKTNTRCTPCGSGTFTSRNNHLPACLSCNGRRDRVTRLTIESVNALPDIIVFSKDHPDARHVFPKQNV

SEQ ID NO: 507 - protein, 102 aa, synthetic construct
RDTAPHAPSDGKCKGNEYKRHNLWPGTYASRLCDSKTNTQCTPCGSGTFTSRNNHLPARLSCNGRRDRVTRLTIESVDALPDIIVFSKDHPDARHVFPKQNV

SEQ ID NO: 508 - protein, 102 aa, synthetic construct
RDIAPHAPSDGKCKDNEYKRHNLCPGTYASRLCDSKTNTQCTPCGSGTFTSRNNHLPACLSCNGRRDRVTLLTIESVNALPDIIVFSKDHPDARHVFPKPNV

SEQ ID NO: 509 - protein, 102 aa, synthetic construct
RDIAPHAPSDGKCKDNEYKRHNLCPGTYASRLCDSKTNTRCTPCGSGTFTSRNNHLPACLSCNGRRDRVTLLTIESVNALPDIIVFSKDHPDARHVFPKQNV

SEQ ID NO: 510 - protein, 103 aa, synthetic construct; identical to SEQ ID NO: 459

SEQ ID NO: 511 - protein, 107 aa, synthetic construct
RDIAPHAPSNGKCKDNEYKRHNLCCLSCPPGTYASRLCDSKTNTQCTPCGSGTFTSRNNHLPACLSCNGRRDRVTLLTIESVNALPDIIVFSKDHPDARHVFPKQNV

SEQ ID NO: 512 - protein, 108 aa, synthetic construct
PTRHSSCGETEYYETELQKCCQKCQPGTKFKSVCTKDSPTVCEACVAGSYSATHNYYENCFSCDKCSGGRKVVKEQCTPTKNTVCGCQGDYEMKTVGGTNMCRKPKRS

SEQ ID NO: 513 - protein, 109 aa, synthetic construct
PTRHSSCGETEYYETELQKCCQKCQPGTKFKSVCTKDSPTVCEACVAGSTYSATHNYYENCFSCDKCSGGRKVVKEQCTPTKNTVCGCQGDYEMKTVGGTNMCRKPKRS

SEQ ID NO: 514 - protein, 109 aa, synthetic construct
KPTRHSSCGETEYYETELQKCCQKCQPGTKFKSVCTKDSPTVCEACVAGSTYSATHNYYENCFSCDKCSGGRKVVKEQCTPTKNTVCGCQGDYEMKTVGGTNMCRKPKR

SEQ ID NO: 515 - protein, 110 aa, synthetic construct
IPTSSLPHAPVNGACDEGEYLDKRHNQCCNQCPPGEFAKVRCNGNDNTKCERCPPHTYTAIPNYSNGCHQCRKCPTGSFDKVKCTGTQNSKCSCLPGWYCATDSSQTEDC

SEQ ID NO: 516 - protein, 114 aa, synthetic construct
VCQHNEVQLGNECCPPCGSGQRVTKVCTDYTSVTCTPCPNGTYVSGPYNCTDCTQCNVTQVMIRNCTSTNNTVCAPKNHTYFPTPGVQHHKQRQQNHTAHITVKQGKSGRHTLA

SEQ ID NO: 517 - protein, 128 aa, synthetic construct
IPTSSLPHAPVNGACDEGEYLDKRHNQCCNRCPPGEFAKVRCNGNDNTKCERCPPHTYTAIPNYSNGCHQCRKCPTGSFDKVKCTGTQNSKCSCLPGWYCATDSSQTEDCRDCIPKRKMSMRILWWNR

SEQ ID NO: 518 - protein, 162 aa, synthetic construct
DIPTTPHSPINGSCDIGEYLDKKSGNCCKMCPPGAFAKVRCTSDNNTECANCPPGTFTSIPNYSNGCHQCRGHCPEGAFDEKRCTTTHDRICKCLPGWFCVTGSASLQCPMCIPKRKCPCGYFGGIDELGNPLCKSCCKGEYCEHIRSYRPGYVPCNLSKCN

SEQ ID NO: 519 - protein, 164 aa, synthetic construct
IHTPLPPHASVNGSCAEGEYLDKTHNRCCNRCPPGEFAKVRCSGSYNTKCERCPHHTYTAIPNYSNGCHQCRKCPKGSFDKVKCTGTQNSKCACLPGWYCATDSSQTEDCRDCVPKRRCPCGYFGGIDKLGNPICKSCCVGKYCDDIRNHRVGPFPPCKLSKCN

SEQ ID NO: 520 - protein, 164 aa, synthetic construct
IPTSSLPHAPVNGACDEGEYLDKRHNQCCNQCPPGEFAKVRCNGNDNTKCERCPPHTYTAIPNYSNGCHQCRKCPTGSFDKVKCTGTQNSKCSCLPGWYCATDSSQTEDCRDCIPKRRCPCGYFGGIDEQGNPICKSCCVGEYCDYLRNYRLDPFPPCKLSKCN

SEQ ID NO: 521 - protein, 164 aa, synthetic construct
IPTPPLPHAPVNGACDEGEYLDKRHNQCCNQCPPGEFAKVRCNGNDNTKCERCPPHTYTAIPNYSNGCHQCRKCPTGSFDKVKCTGTQNSKCSCLPGWYCATDSSQTEDCRDCIPKRRCPCGYFGGIDEQGNPICKSCCVGEYCDYLRNYRLDPFPPCKLSKCN

SEQ ID NO: 522 - protein, 332 aa, synthetic construct
ITPHEPSNGKCKDNEYKHHHLCCLSCPPGTYASRLCDSKTNTNTQCTPCGSGTFTSRNNHLPACLSCNGRCDSNQVETRSCNTTHNRICDCAPGYYCLLKGSSGCKACVSQTKCGIGYGVSGHTPTGDVVCSPCGLGTYSHTVSSVDKCEPVPSNTFNYIDVEINLYPVNDTSCTRTTTTGLSESISTSELTITMNHKDCDPVFRDGYFSVLNKVATSGFFTGQNRYQNISKVCTLNFEIKCNNKDSYSSSKQLTKAKNDDDSIMPHSETVTLVGDCLSSVDIYILYSNTNTQDYETDTISYHVGNVLDVDSHMPGSCDIHKLITNSSHSLL

SEQ ID NO: 523 - protein, 107 aa, synthetic construct
MNIIFLSAIVTCLVYTTFGKTCPADYYLEPEDGLCTACVTCLSNMVEIQPCGPDKPRKCQCGPGLKCTLPAVNSCARCTPDTTTKKVQKEQCCTTPDNTKLCYHKYS

SEQ ID NO: 524 - protein, 121 aa, synthetic construct
MTIHILDVLNSMKMNTIFLSAIVTCIVYTTFGKTCPADYYLEPEDGLCTACVTCLSNMVEIQPCGPDKPRKCQCGPGLKCTVPAVNSCARCTPDTTTKKIEPIGQCCTTPDNTKLCYHKYS

SEQ ID NO: 525 - protein, 115 aa, synthetic construct
MNIIFLSAIVSCLVYTTFGGKTCKTCPADYYLNPENGLCTACVTCLSNMVEIQPCGPDKPRKCECGSGFKCTLPAVNSCARCTPDTTTKKVQKEQKEQCCNTPDNTKLCYHKYSS

SEQ ID NO: 526 - protein, 110 aa, synthetic construct
MKMNTIFLSAIVTCIVYTSFGKTCPNDYYLEPEDGLCTACVTCLSNMVEIQPCGPDKPRKCKCGPGLKCTVPAVNSCARCTPDTTTKKIEPIGQCCTTPDNTKLCYHKYS

SEQ ID NO: 527 - protein, 110 aa, synthetic construct
MKMNTIFLSAIVTCLVYTSFGKTCPNDYYLEPEDGLCTACVTCLSNMVEIQPCGPDKPRKCQCGPGLKCTVPAVNSCARCTPDTTTKKIEPIGQCCTTPDNTKLCYHKYS

SEQ ID NO: 528 - protein, 109 aa, synthetic construct
MKMNIIFLSAIVTCLVYTTFGKTCPADYYLEPEDGLCTACVTCLSNMVETQSCGPDKPRKCQCGPGLKCTVPAVNSCARCTPDTTTKKVQKEQCCTTPDNTKLCYHKYS

SEQ ID NO: 529 - protein, 108 aa, synthetic construct
MKMNTIFLSAIVTCIVYTSFGKTCPNDYYLEPEDGLCTACVTCLSNMVEIQPCGPDKPRKCQCGPGLKCTVPAVNSCARCTPDTTIKKIKPTDQCCTTPDNTKLCYHK

SEQ ID NO: 530 - protein, 111 aa, synthetic construct
MKMNTIFLSAIVTCLVYTSFGKTCPNDYYLEPEDGLCTACVTCLSNMVEIQPCGPDKPRKCQCGPGLKCTVPAVNSCARCTPDTTIKKIEPTDQCCTTPDNTKLCYHKYSP

SEQ ID NO: 531 - protein, 110 aa, synthetic construct
MKMNTIFLSAIVTCIVYTSFGKTCPADYYLEPEDGLCTACVTCLSNMVEIQPCGPDKPRKCQCGPGLKCTVPAVNSCARCTPDTTTKKVQKDQCCTTPDNTKLCYHKYSS

SEQ ID NO: 532 - protein, 118 aa, synthetic construct
MNKFIVLSTIVSCLVYTSFGKTCPVDHYYDREDGGLCTACVTCLQNMVEIQPCGPDKPRKCQCAPGFKCTLPAVNSCARCTPDSTYKPPPKPATKPKSHEDNCCITTGNTKLCYTQLN

SEQ ID NO: 533 - protein, 103 aa, synthetic construct
MKYILLITICYSLYCISFASDCEKDYYRDPKTGRCTACVTCTGDTVEKSPCGPTTPRKCECGPGLKCTVSATNTCARCEEHIPDNCCKTKDNTKLCYQKLRGS

SEQ ID NO: 534 - protein, 110 aa, synthetic construct
MKMNIIFLSAIVTCLVYTTFGKTCPADYYLEPEDGLCTACVTCLSNMVETQSCGPDKPRKCQCGPGLKCTVPAVNSCARCTPDTTTKKVQKEQCCTTPDNTKLCYHKYSS

SEQ ID NO: 535 - protein, 101 aa, synthetic construct
MKPQILFLCACVILMASNKKADARTCTEGSFLNSLDKKCYDCPYNTWQASRRHSEIVCNMCTQCKPGYTLTQKCTPTTDTVCMCRKPFVEKNGFCMLFHKP

SEQ ID NO: 536 - protein, 103 aa, synthetic construct
MKYILLITICYSLYCISFASQCEADYYKDPTTNRCTACVTCGGDMVETAPCGPNTPRKCECGPGLKCALAVQNSCARCEEYIPDNCCKTKDKTKLCYEKLRGS

SEQ ID NO: 537 - protein, 110 aa, synthetic construct
MKMNTIFLSAIVTCLVYTSFGKTCPNDYYLEPEDGLCKACVTCLSNMVEIQPCGPDKPRKCQCGPGLKCTVPVVNSCARCTPDTTTKKVQKEQCCTTPDNTKLCYHKYSS

SEQ ID NO: 538 - protein, 110 aa, synthetic construct
MKMNIIFLSAIVTCLVYTTFGKTCPADYYLEPEDGLCTACVTCLSNMVEIQPCGPDKPRKCQCGPGLKCTVPAVNSCARCTPDTTTKKVQKEQCCTTPDNTKLCYHKYSS

SEQ ID NO: 539 - protein, 111 aa, synthetic construct
MKMNTIFLSAIVTCLVYTSFGKTCPNDYYLEPEDGLCTACVTCLSNMVEIQPCGPNKPRKCQCGPGLKCTVPAVNSCARCTPDTTIKKIEPTDQCCTTPDNTKLCYHKYSS

SEQ ID NO: 540 - protein, 111 aa, synthetic construct
MKMNTIFLSAIVTCLVYTSFGKTCPNDYYLEPEDGLCTACVTCLSNMVEIQPCGPDKPRKCQCGPGLKCTVPAVNSCARCTPDTTIKKIEPTDQCCTTPDNTKLCYHKYSS

SEQ ID NO: 541 - protein, 111 aa, synthetic construct
MKMNTIFLSAIVTCLVYTSFGKTCPNDYYLEPEDGLCKACVTCLSNMVEIQSCGPDKPRKCQCGPGLKCMLPAVNSCARCTPDTTTKKIEPTEQCCTTPDNTKLCYHKYSS

SEQ ID NO: 542 - protein, 109 aa, synthetic construct
MNIIFLSAIVTCLVYTTFGKTCPADYYLEPEDGLCTACVTCLSNMVETQPCGPDKPRKCQCGPGLKCTLPAVNSCARCTPDTTTKKVQKEQCCTTPDNTKLCYHKYSSS

SEQ ID NO: 543 - protein, 109 aa, synthetic construct
MNTIFLSAIVTCLVYTSFGKTCPNDYYLEPEDGLCTACVTCLSNMVEIQPCGPDKPRKCQCGPGLKCTVPAVNSCARCTPDTTTKKIEPIGQCCTTPDNTKLCYHKYSS

SEQ ID NO: 544 - protein, 111 aa, synthetic construct
MKMNTIFLSAIVTCLVYTSFGKTCPNDYYLEPEDGLCKACVTCLSNMVEIQSCGPDKPRKCQCGPGLKCTLPAVNSCARCTPDTTTKKIEPTEQCCTTPDNTKLCYHKYSS

SEQ ID NO: 545 - protein, 109 aa, synthetic construct
MNTIFLSAIVTCIVYTTFGKTCPADYYLEPEDGLCTACVTCLSNMVEIQPCGPDKPRKCQCGPGLKCTVPAVNSCARCTPDTTTKKIEPIGQCCTTPDNTKLCYHKYSS

SEQ ID NO: 546 - protein, 109 aa, synthetic construct
MNTIFLSAIVTCIVYTSFGKTCPNDYYLEPEDGLCTACVTCLSNMVEIQPCGPDKPRKCKCGPGLKCTVPAVNSCARCTPDTTTKKIEPIGQCCTTPDNTKLCYHKYSS

SEQ ID NO: 547 - protein, 111 aa, synthetic construct
MNIIFLSAIVSCLVYTTFGKTCPADYYLNPENGLCTACVTCLSNMVEIQPCGPDKPRKCECGSGFKCTLPAVNSCARCTPDTTTKKVQKEQKEQCCNTPDNTKLCYHKYSS

SEQ ID NO: 548 - protein, 118 aa, synthetic construct
MGYKGIYKIIFFSTIMYSLVYCKICQNDYYFNKETGHCTACVTCVGNTKETHPCGPDEPRTCECDSGFKCDLPVANSCARCIVIPPTKSTKNKHIKEQCCNTTDNVKLCYTIQNSFIK

SEQ ID NO: 549 - protein, 119 aa, synthetic construct
MNKFIIVYTIVSYLVYTSFGKTCPTDYYYNREDDGLCTACVTCLKNMVEIQPCGPDKPRKCQCAPGFKCTVPAVNSCARCTPDSTYKPPIPKPASKPKSPDDNCCVTTSNIKLCYTQLN

SEQ ID NO: 550 - protein, 119 aa, synthetic construct
MKSVLYSYILFLSCIIINGRDIAPHAPSDGKCKDNEYKRHNLCPGTYASRLCDSKTNTQCTPCTFTSRNNHLPACLSCNGRRDRVTRLTIESVNALPDIIVFSKDHPDARHVFPKQNVE

SEQ ID NO: 551 - protein, 119 aa, synthetic construct; identical to SEQ ID NO: 463

SEQ ID NO: 552 - protein, 118 aa, synthetic construct
MKSVLYLYILFLSCIIINGRDIAPHAPSDGKCKDNEYKRHNLCPGTYASRLCDSKTNTQCTPCTFTSRNNHLPACLSCNGRRDRVTRLTIESVNALPDIIVFSKDHPDARHVFPKQNV

SEQ ID NO: 553 - protein, 141 aa, synthetic construct
MIERKYLYDWRSRKQEQDDDYYIIKSVLYSYILFLSCIIINGRDIAPHAPSDGKCKDNEYKRHNLCPGTYASRLCDSKTNTQCTPCTFTSRNNHLPACLSCNGRRDRVTLLTIESVNALPDIIVFSKDHPDARHVFPKQNV

SEQ ID NO: 554 - protein, 126 aa, synthetic construct
MKPLMMLICFGVFLLQLGGSKMCKPDEVKLGNQCCPPCGSGQKVTKVCTENSGITCTLCPNGTYLTGLYNCTNCTQCNDTQITVRNCTSTNNTICASKNHTLFSTPGVQHHKQRQQNHTAHVTVKQ

SEQ ID NO: 555 - protein, 122 aa, synthetic construct
MKSVLYSYILFLSCIIINGRDIAPHAPSNGKCKDNEYKRHNLCPGTYASRLCDSKTNTRCTPCGSGTFTSRNNHLPACLSCNGRRDRVTLLTIESVNALPDIIVFSKDHPDARHVFPKQNVE

SEQ ID NO: 556 - protein, 122 aa, synthetic construct
DIAPHAPSDGKCKDNEYKRHNLCPGTYASRLCDSKTNTQCTPCGSGMKSVLYSYILFLSCIIINGRTFTSRNNHLPACLSCNGRRDRVTRLTIESVNALPDIIVFSKDHPDARHVFPKQNVE

SEQ ID NO: 557 - protein, 122 aa, synthetic construct
MKSVLYSYILFLSCIIINGRDIAPHAPSDGKCKDNEYKRHNLCPGTYASRLCDSKTNTRCTPCGSGTFTSRNNHLPACLSCNGRRDRVTRLTIESVNALPDIIVFSKDHPDARHVFPKQNVE

SEQ ID NO: 558 - protein, 121 aa, synthetic construct
MKSVLYSYILFLSCIIINGRDIAPHAPSDGKCKDNEYKRHNLCPGTYASRLCDSKTNTQCTPCGSGTFTSRNNHLPACLSCNGRRDRVTRLTIESVNALPDIIVFSKDHPDARHVFPKQNV

SEQ ID NO: 559 - protein, 121 aa, synthetic construct
MKSVLYLYILFLSCIIINGRDIAPHAPSDGKCKDNEYKRHNLCPGTYASRLCDSKTNTQCTPCGSGTFTSRNNHLPACLSCNGRCDRVTLLTIESVNALPDIIVFSKDHPDARHVFPKQNV

SEQ ID NO: 560 - protein, 122 aa, synthetic construct
MKSVLYSYILFLSCIIINGERDIAPHAPSNGKCKDNEYKRHNLCPGTYASRLCDSKTNTRCTPCGSGTFTSRNNHLPACLSCNGRRDRVTRLTIESVNALPDIIVFSKDHPDARHVFPKQNV

SEQ ID NO: 561 - protein, 121 aa, synthetic construct
MKSVLYSYILFLSFIIINGRDTAPHAPSDGKCKGNEYKRHNLWPGTYASRLCDSKTNTQCTPCGSGTFTSRNNHLPARLSCNGRRDRVTRLTIESVDALPDIIVFSKDHPDARHVFPKQNV

SEQ ID NO: 562 - protein, 121 aa, synthetic construct
MKSVLYLYILFLSCIIINGRDIAPHAPSDGKCKDNEYKRHNLCPGTYASRLCDSKTNTQCTPCGSGTFTSRNNHLPACLSCNGRRDRVTLLTIESVNALPDIIVFSKDHPDARHVFPKPNV

SEQ ID NO: 563 - protein, 144 aa, synthetic construct
MIERKYLYDWRSRKQEQDNDYYIIKSVLYSYILFLSCIIINGRDIAPHAPSDGKCKDNEYKRHNLCPGTYASRLCDSKTNTRCTPCGSGTFTSRNNHLPACLSCNGRRDRVTLLTIESVNALPDIIVFSKDHPDARHVFPKQNV

SEQ ID NO: 564 - protein, 124 aa, synthetic construct; identical to SEQ ID NO: 464

SEQ ID NO: 565 - protein, 126 aa, synthetic construct
MKSVLYLYILFLSCIIINGRDIAPHAPSNGKCKDNEYKRHNLCCLSCPPGTYASRLCDSKTNTQCTPCGSGTFTSRNNHLPACLSCNGRRDRVTLLTIESVNALPDIIVFSKDHPDARHVFPKQNV

SEQ ID NO: 566 - protein, 127 aa, synthetic construct
MKLLVILGLGLCYLAVQAKPTRHSSCGETEYYETELQKCCQKCQPGTKFKSVCTKDSPTVCEACVAGSYSATHNYYENCFSCDKCSGGRKVVKEQCTPTKNTVCGCQGDYEMKTVGGTNMCRKPKRS

SEQ ID NO: 567 - protein, 128 aa, synthetic construct
MKLLVILGLGLCYLAVQAKPTRHSSCGETEYYETELQKCCQKCQPGTKFKSVCTKDSPTVCEACVAGSTYSATHNYYENCFSCDKCSGGRKVVKEQCTPTKNTVCGCQGDYEMKTVGGTNMCRKPKRS

SEQ ID NO: 568 - protein, 165 aa, synthetic construct
MTHTVTSAECLLLSVSVLNKISCFTILYHISDLISSQTMKLLVILGLGLCYLAVQAKPTRHSSCGETEYYETELQKCCQKCQPGTKFKSVCTKDSPTVCEACVAGSTYSATHNYYENCFSCDKCSGGRKVVKEQCTPTKNTVCGCQGDYEMKTVGGTNMCRKPKR

SEQ ID NO: 569 - protein, 132 aa, synthetic construct
MDIKNLLTACTIFYITTLATADIPTSSLPHAPVNGACDEGEYLDKRHNQCCNQCPPGEFAKVRCNGNDNTKCERCPPHTYTAIPNYSNGCHQCRKCPTGSFDKVKCTGTQNSKCSCLPGWYCATDSSQTEDC

SEQ ID NO: 570 - protein, 135 aa, synthetic construct
MKPLIMLICFAVILLQLGVTKVCQHNEVQLGNECCPPCGSGQRVTKVCTDYTSVTCTPCPNGTYVSGPYNCTDCTQCNVTQVMIRNCTSTNNTVCAPKNHTYFPTPGVQHHKQRQQNHTAHITVKQGKSGRHTLA

SEQ ID NO: 571 - protein, 150 aa, synthetic construct
MDIKNLLTVCTIFYITTLATADIPTSSLPHAPVNGACDEGEYLDKRHNQCCNRCPPGEFAKVRCNGNDNTKCERCPPHTYTAIPNYSNGCHQCRKCPTGSFDKVKCTGTQNSKCSCLPGWYCATDSSQTEDCRDCIPKRKMSMRILWWNR

SEQ ID NO: 572 - protein, 184 aa, synthetic construct
MMDINYRLAICTILYIGTIVATDIPTTPHSPINGSCDIGEYLDKKSGNCCKMCPPGAFAKVRCTSDNNTECANCPPGTFTSIPNYSNGCHQCRGHCPEGAFDEKRCTTTHDRICKCLPGWFCVTGSASLQCPMCIPKRKCPCGYFGGIDELGNPLCKSCCKGEYCEHIRSYRPGYVPCNLSKCN

SEQ ID NO: 573 - protein, 186 aa, synthetic construct
MDIKSLLAVCTILYITTLVTADIHTPLPPHASVNGSCAEGEYLDKTHNRCCNRCPPGEFAKVRCSGSYNTKCERCPHHTYTAIPNYSNGCHQCRKCPKGSFDKVKCTGTQNSKCACLPGWYCATDSSQTEDCRDCVPKRRCPCGYFGGIDKLGNPICKSCCVGKYCDDIRNHRVGPFPPCKLSKCN

SEQ ID NO: 574 - protein, 186 aa, synthetic construct
MDIKNLLTACTIFYITTLATADIPTSSLPHAPVNGACDEGEYLDKRHNQCCNQCPPGEFAKVRCNGNDNTKCERCPPHTYTAIPNYSNGCHQCRKCPTGSFDKVKCTGTQNSKCSCLPGWYCATDSSQTEDCRDCIPKRRCPCGYFGGIDEQGNPICKSCCVGEYCDYLRNYRLDPFPPCKLSKCN

SEQ ID NO: 575 - protein, 186 aa, synthetic construct
MDIKNLLTVCTIFYITTLATADIPTPPLPHAPVNGACDEGEYLDKRHNQCCNQCPPGEFAKVRCNGNDNTKCERCPPHTYTAIPNYSNGCHQCRKCPTGSFDKVKCTGTQNSKCSCLPGWYCATDSSQTEDCRDCIPKRRCPCGYFGGIDEQGNPICKSCCVGEYCDYLRNYRLDPFPPCKLSKCN

SEQ ID NO: 576 - protein, 351 aa, synthetic construct
MKSYILLLLLSYIIIINSDITPHEPSNGKCKDNEYKHHHLCCLSCPPGTYASRLCDSKTNTNTQCTPCGSGTFTSRNNHLPACLSCNGRCDSNQVETRSCNTTHNRICDCAPGYYCLLKGSSGCKACVSQTKCGIGYGVSGHTPTGDVVCSPCGLGTYSHTVSSVDKCEPVPSNTFNYIDVEINLYPVNDTSCTRTTTTGLSESISTSELTITMNHKDCDPVFRDGYFSVLNKVATSGFFTGQNRYQNISKVCTLNFEIKCNNKDSYSSSKQLTKAKNDDDSIMPHSETVTLVGDCLSSVDIYILYSNTNTQDYETDTISYHVGNVLDVDSHMPGSCDIHKLITNSSHSLL

SEQ ID NO: 577 - protein, 1032 aa, Homo sapiens [sequence omitted]

SEQ ID NO: 578 - protein, 999 aa, Homo sapiens
YNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAYHLGPHVISKRSTEEFPPLQPILQQKKEKDIMKKTINFARFCAHENCSADLQVSAKIGFLKPHENKTYLAVGSMKTLMLNVSLFNAGDDAYETTLHVKLPVGLYFIKILELEEKQINCEVTDNSGVVQLDCSIGYIYVDHLSRIDISFLLDVSSLSRAEEDLSITVHATCENEEEMDNLKHSRVTVAIPLKYEVKLTVHGFVNPTSFVYGSNDENEPETCMVEKMNLTFHVINTGNSMAPNVSVEIMVPNSFSPQTDKLFNILDVQTTTGECHFENYQRVCALEQQKSAMQTLKGIVRFLSKTDKRLLYCIKADPHCLNFLCNFGKMESGKEASVHIQLEGRPSILEMDETSALKFEIRATGFPEPNPRVIELNKDENVAHVLLEGLHHQRPKRYFTIVIISSSLLLGLIVLLLISYVMWKAGFFKRQYKSILQEENRRDSWSYINSKSNDD

SEQ ID NO: 579 - protein, 798 aa, Homo sapiens
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 580 - protein, 779 aa, Homo sapiens
ELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 581 - protein, 382 aa, Homo sapiens
MDFGLALLLAGLLGLLLGQSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 582 - protein, 364 aa, Homo sapiens
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQLPAALWTSSAVLGLLLLALPTYHLWKRCRHLAEDDTHPPASLRLLPQVSAWAGLRGTGQVGISPS

SEQ ID NO: 583 - protein, 5 aa, synthetic construct
SYWMH

SEQ ID NO: 584 - protein, 17 aa, synthetic construct
EIDPSESNTNYNQKFKG

SEQ ID NO: 585 - protein, 12 aa, synthetic construct
GGYDGWDYAIDY

SEQ ID NO: 586 - protein, 16 aa, synthetic construct
RSSQSLAKSYGNTYLS

SEQ ID NO: 587 - protein, 7 aa, synthetic construct
GISNRFS

SEQ ID NO: 588 - protein, 9 aa, synthetic construct
LQGTHQPYT

SEQ ID NO: 589 - protein, 121 aa, synthetic construct [sequence omitted]

SEQ ID NO: 590 - protein, 115 aa, synthetic construct
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTV

SEQ ID NO: 591 - protein, 451 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 592 - protein, 219 aa, synthetic construct
DVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 593 - protein, 548 aa, synthetic construct
MYRMQLLSCIALSLALVTNSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 594 - protein, 528 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 595 - protein, 547 aa, synthetic construct
MYRMQLLSCIALSLALVTNSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQSGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 596 - protein, 527 aa, synthetic construct
KGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQSGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 597 - protein, 479 aa, synthetic construct
MYRMQLLSCIALSLALVTNSSTYRVRSSGLTCSTCPPGTHKERDCSLNTETICKACGEGEYTAHNNSLPKCLACKSCFNATEIETKSCDPTSDTICACREGYSINNLGECNGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 598 - protein, 459 aa, synthetic construct
STYRVRSSGLTCSTCPPGTHKERDCSLNTETICKACGEGEYTAHNNSLPKCLACKSCFNATEIETKSCDPTSDTICACREGYSINNLGECNGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 599 - protein, 467 aa, synthetic construct
MYRMQLLSCIALSLALVTNSSTYRVRSSGLTCSTCPPGTHKERDCSLNTETICKACGEGEYTAHNNSLPKCLACKSCFNATEIETKSCDPTSDTICACREGYSINNLGECNGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 600 - protein, 447 aa, synthetic construct
STYRVRSSGLTCSTCPPGTHKERDCSLNTETICKACGEGEYTAHNNSLPKCLACKSCFNATEIETKSCDPTSDTICACREGYSINNLGECNGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 601 - protein, 491 aa, synthetic construct
MYRMQLLSCIALSLALVTNSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 602 - protein, 471 aa, synthetic construct
DSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 603 - protein, 468 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECNGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 604 - protein, 448 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECNGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 605 - protein, 492 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 606 - protein, 472 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 607 - protein, 479 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECNGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 608 - protein, 459 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECNGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 609 - protein, 479 aa, synthetic construct
MYRMQLLSCIALSLALVTNSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 610 - protein, 459 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 611 - protein, 479 aa, synthetic construct
MYRMQLLSCIALSLALVTNSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 612 - protein, 459 aa, synthetic construct
DSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 613 - protein, 1076 aa, synthetic construct
MYRMQLLSCIALSLALVTNSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 614 - protein, 1056 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 615 - protein, 639 aa, synthetic construct
MYRMQLLSCIALSLALVTNSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSGGGGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 616 - protein, 619 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSGGGGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 617 - protein, 592 aa, synthetic construct
MYRMQLLSCIALSLALVTNSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 618 - protein, 572 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 619 - protein, 580 aa, synthetic construct
MYRMQLLSCIALSLALVTNSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 620 - protein, 560 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 621 - protein, 829 aa, synthetic construct
MYRMQLLSCIALSLALVTNSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 622 - protein, 809 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSDVVMTQSPLSLPVTPGEPASISCRSSQSLAKSYGNTYLSWYLQKPGQSPQLLIYGISNRFSGVPDRFSGSGSGTDFTLKISRVEAEDVGVYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 623 - protein, 701 aa, synthetic construct
MYRMQLLSCIALSLALVTNSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 624 - protein, 681 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 625 - protein, 675 aa, synthetic construct
MYRMQLLSCIALSLALVTNSQVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 626 - protein, 655 aa, synthetic construct
QVQLVQSGAEVKKPGASVKVSCKGSGYTFTSYWMHWVRQAPGQRLEWIGEIDPSESNTNYNQKFKGRVTLTVDISASTAYMELSSLRSEDTAVYYCARGGYDGWDYAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCIGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 627 - protein, 350 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSDSKCGVSEYYNKEHDICCRLCPAGSYAEQLCTKDNDTVCNQCPPNTFLSIPNYISSCLSCRGKCINDHVEDKPCTATSNRICKCKENKTCVLKTYDNSCRVCI

SEQ ID NO: 628 - protein, 229 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 629 - protein, 407 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 630 - protein, 406 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSKGRDSKPSPACDPMHGALAGIFKELRTTYRSVREALQTKDTVYYVSLFHEQLLQEMLSPVGCRVTNELMQHYLDGVLPRAFHCGYDNATLNALHALSSSLSTLYQHMLKCPALACTGQTPAWTQFLDTEHKLDPWKGTVKATAEMDLLLNYLETFLLQS

SEQ ID NO: 631 - protein, 338 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGGGGSGGGGSGGGGSSTYRVRSSGLTCSTCPPGTHKERDCSLNTETICKACGEGEYTAHNNSLPKCLACKSCFNATEIETKSCDPTSDTICACREGYSINNLGECN

SEQ ID NO: 632 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 633 - protein, 12 aa, synthetic construct
EPKSCDKTHTCP

SEQ ID NO: 634 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 635 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 636 - protein, 106 aa, synthetic construct [sequence omitted]

SEQ ID NO: 637 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 638 - protein, 219 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 639 - protein, 223 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 640 - protein, 222 aa, synthetic construct
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 641 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 642 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 643 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 644 - protein, 231 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 645 - protein, 330 aa, synthetic construct [sequence omitted]

SEQ ID NO: 646 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 647 - protein, 98 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 648 - protein, 12 aa, synthetic construct
ESKYGPPCPSCP

SEQ ID NO: 649 - protein, 13 aa, synthetic construct
AESKYGPPCPSCP

SEQ ID NO: 650 - protein, 110 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 651 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 652 - protein, 106 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 653 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 654 - protein, 216 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 655 - protein, 223 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 656 - protein, 222 aa, synthetic construct
PCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 657 - protein, 229 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 658 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 659 - protein, 230 aa, synthetic construct
AESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 660 - protein, 229 aa, synthetic construct
AESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 661 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 662 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 663 - protein, 107 aa, synthetic construct [sequence omitted]

SEQ ID NO: 664 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 665 - protein, 97 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKI

SEQ ID NO: 666 - protein, 13 aa, synthetic construct
VPRDCGCKPCICT

SEQ ID NO: 667 - protein, 107 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTK

SEQ ID NO: 668 - protein, 107 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 669 - protein, 106 aa, synthetic construct
GRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 670 - protein, 214 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 671 - protein, 213 aa, synthetic construct
VPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 672 - protein, 227 aa, synthetic construct
VPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 673 - protein, 227 aa, synthetic construct; identical to SEQ ID NO: 672

SEQ ID NO: 674 - protein, 16 aa, synthetic construct
EPRGPTIKPCPPCKCP

SEQ ID NO: 675 - protein, 134 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVT

SEQ ID NO: 676 - protein, 83 aa, synthetic construct
LTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 677 - skipped

SEQ ID NO: 678 - protein, 82 aa, synthetic construct
LTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 679 - protein, 217 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 680 - protein, 216 aa, synthetic construct
APNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 681 - protein, 233 aa, synthetic construct
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 682 - protein, 232 aa, synthetic construct
EPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 683 - protein, 330 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIEPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 684 - protein, 329 aa, synthetic construct
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIEPRGPTIKPCPPCKCPAPNAAGGPSVFIFLLKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 685 - protein, 107 aa, synthetic construct
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 686 - protein, 105 aa, synthetic construct
QPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 687 - protein, 220 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 688 - protein, 219 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 689 - protein, 223 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 690 - protein, 222 aa, synthetic construct
TCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 691 - protein, 232 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 692 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 693 - protein, 217 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 694 - protein, 216 aa, synthetic construct
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 695 - protein, 223 aa, synthetic construct
PCPSCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 696 - protein, 222 aa, synthetic construct
PCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 697 - protein, 229 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 698 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 699 - protein, 230 aa, synthetic construct
AESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 700 - protein, 229 aa, synthetic construct; identical to SEQ ID NO: 628

SEQ ID NO: 701 - protein, 8 aa, synthetic construct
GGGGGGGS

SEQ ID NO: 702 - protein, 16 aa, synthetic construct
GGGGGGGSGGGGGGGS

SEQ ID NO: 703 - protein, 24 aa, synthetic construct
GGGGGGGSGGGGGGGSGGGGGGGS

SEQ ID NO: 704 - protein, 5 aa, synthetic construct
GGGGS

SEQ ID NO: 705 - protein, 10 aa, synthetic construct
GGGGSGGGGS

SEQ ID NO: 706 - protein, 15 aa, synthetic construct
GGGGSGGGGSGGGGS

SEQ ID NO: 707 - protein, 4 aa, synthetic construct
GGGS

SEQ ID NO: 708 - protein, 8 aa, synthetic construct
GGGSGGGS

SEQ ID NO: 709 - protein, 12 aa, synthetic construct
GGGSGGGSGGGS

SEQ ID NO: 710 - protein, 18 aa, synthetic construct
GGGGGGGSGGGGSGGGGS

SEQ ID NO: 711 - protein, 19 aa, synthetic construct
GGGGGGGGSGGGGSGGGGS

SEQ ID NO: 712 - protein, 20 aa, Homo sapiens
MYRMQLLSCIALSLALVTNS

SEQ ID NO: 713 - protein, 25 aa, Homo sapiens
MGVLLTQRTLLSLVLALLFPSMASM

SEQ ID NO: 714 - protein, 18 aa, Homo sapiens
MASLWLLSCFSLVGAAFG

SEQ ID NO: 715 - protein, 15 aa, Homo sapiens
MNLLLILTFVAAAVA

SEQ ID NO: 716 - protein, 24 aa, Homo sapiens
MALWMRLLPLLALLALWGPDPAAA

(19) United States
(12) Patent Application Publication
(10) Pub. No.: US 2026/0015407 A1
(43) Pub. Date: Jan. 15, 2026

(54) IMMUNOMODULATORY FUSION PROTEINS AND RELATED METHODS

(71) Applicant: Flagship Pioneering Innovations VII, LLC, Cambridge, MA (US)

(72) Inventors: Hozefa Saifuddin BANDUKWALA, Southborough, MA (US); Shruti PRATAPA, Sudbury, MA (US); Angela Wynne NORTON, Reading, MA (US); Samuel Clement HASSAN, Somerville, MA (US)

(21) Appl. No.: 19/267,934

(22) Filed: Jul. 14, 2025

Related U.S. Application Data
(60) Provisional application No. 63/671,604, filed on Jul. 15, 2024.
(60) Provisional application No. 63/671,614, filed on Jul. 15, 2024.
(60) Provisional application No. 63/684,935, filed on Aug. 20, 2024.
(60) Provisional application No. 63/715,908, filed on Nov. 4, 2024.
(60) Provisional application No. 63/715,937, filed on Nov. 4, 2024.

(51) Int. Cl. C07K 14/715; C07K 14/705; C07K 16/28

(52) U.S. Cl. CPC C07K 14/7151; C07K 14/70575; C07K 14/7155; C07K 16/2839; C07K 2319/00

(57) ABSTRACT

Provided herein are immunomodulatory fusion proteins and compositions (e.g., pharmaceutical compositions) comprising the same; as well as methods of making the immunomodulatory fusion proteins and compositions. The immunomodulatory fusion proteins provided herein are useful in pharmaceutical compositions and methods of use (including, e.g., methods of suppressing or reducing (e.g., preventing) a pro-inflammatory immune response in a subject).

### RELATED APPLICATIONS

This application to claims priority to and the benefit of U.S. Ser. No. 63/671,604, filed Jul. 15, 2024; U.S. Ser. No. 63/671,614, filed Jul. 15, 2024; U.S. Ser. No. 63/684,935, filed Aug. 20, 2024; U.S. Ser. No. 63/715,908, filed Nov. 4, 2024; U.S. Ser. No. 63/715,937, filed Nov. 4, 2024; the entire contents of each of which is incorporated herein by reference.

### SEQUENCE LISTING

The instant application contains a Sequence Listing which has been submitted electronically in XML format and is hereby incorporated by reference in its entirety. Said XML copy, created on Jul. 10, 2025, is named 62801_72US01_SL.xml and is 745,472 bytes in size.

### 1. FIELD

This disclosure relates to immunomodulatory fusion proteins and nucleic acid molecules encoding the same, as well as pharmaceutical compositions comprising any of the foregoing. The disclosure further relates to methods of making and utilizing the same, including, e.g., methods of suppressing or reducing (e.g., preventing) a pro-inflammatory immune response.

### 2. BACKGROUND

Fusion proteins are a class of engineered proteins that comprise at least one protein operably connected to another protein, wherein the two proteins are not naturally found operably connected. Fusion proteins can be utilized for example, to add one or more functionality to a single protein. For example, fusion proteins are commonly utilized as detection reagents (e.g., florescent proteins fused to a targeting protein (e.g., an antibody that binds a specific target antigen)). Fusion proteins can also be utilized as, e.g., therapeutic, and diagnostic agents.

### 3. SUMMARY

Provided herein are, inter alia, immunomodulatory fusion proteins (and nucleic acid molecules encoding the same) and combinations regimens of immunomodulatory proteins (and fusion proteins thereof); methods of manufacturing the same; pharmaceutical compositions comprising the same; and methods of use including e.g., suppressing or reducing (e.g., preventing) an immune response; and methods of suppressing or reducing (e.g., preventing) a pro-inflammatory immune response.

Accordingly, provided herein are, inter alia, fusion protein comprising (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and any one or more (e.g., 1, 2, 3, or 4) of (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576; and/or (a-1) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386.

Also, provided herein are, inter alia, fusion proteins comprising (a) an integrin binding domain; and one or more of (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and/or (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

### 4. BRIEF DESCRIPTION OF THE FIGURES

FIG. 1A is a graphical representation of an exemplary format of an immunomodulatory fusion protein described herein, wherein the C-terminus of a first immunomodulatory protein described herein is operably connected indirectly through a peptide linker to the N-terminus of an Ig (e.g., hIg) Fc region, and the N-terminus of a second immunomodulatory protein described herein is operably connected indirectly through a peptide linker to the C-terminus of the Ig (e.g., hIg) Fc region.

FIG. 1B is a graphical representation of an exemplary format of an immunomodulatory fusion protein described herein, wherein the N-terminus of a first immunomodulatory protein described herein is operably connected indirectly through a peptide linker to the C-terminus a full-length antibody, and the N-terminus of a second immunomodulatory protein described herein is operably connected indirectly through a peptide linker to the C-terminus of the first immunomodulatory protein.

FIG. 2 is a line graph showing the % of IL-10 activity in cell cultures treated with the indicated agent (IFP-1, Fc-control, or a control IL-10 binder (hIL-10 control)) at the indicated concentration.

FIG. 3 is a is a line graph showing the % of TNFα activity in cell cultures treated with the indicated agent (IFP-1, Fc-control, or a control TNFα binder (TNFα control)) at the indicated concentration.

FIG. 4 is a line graph showing the % of IL-10 activity in cell cultures treated with the indicated agent (IFP-2, Fc-control, or a control IL-10 binder (IL-10 control)) at the indicted concentration.

FIG. 5 is a is a line graph showing the % of TNFα activity in cell cultures treated with the indicated agent (IFP-10, a control TNFα binder (TNFα control), a control TL1A binder (TL1A control)) at the indicated concentration.

FIG. 6 is a is a line graph showing the NFκB activity (as a measure of TL1A inhibition) in cell cultures treated with the indicated agent (IFP-10, a control TNFα binder (TNFα control), or a control TL1A binder (TL1A control)) at the indicated concentration.

FIG. 7A is a graphical representation of an exemplary format of an immunomodulatory fusion protein described herein, wherein the N-terminus of a first immunomodulatory protein described herein is indirectly operably connected to the C-terminus of one of the heavy chains a full-length anti-integrin (e.g., α4β7 integrin) antibody through a peptide linker, and the N-terminus of a second immunomodulatory protein described herein is indirectly operably connected to the C-terminus of the other the heavy chain of the full-length anti-integrin (e.g., α4β7 integrin) antibody through a peptide linker. In some embodiments, the first and second immunomodulatory proteins have an identical amino acid sequence.

FIG. 7B is a graphical representation of an exemplary format of an immunomodulatory fusion protein described herein, wherein the N-terminus of a first immunomodulatory protein described herein is indirectly operably connected to the C-terminus of one of the heavy chains a full-length anti-integrin (e.g., α4β7 integrin) antibody through a peptide linker; the N-terminus of a second immunomodulatory protein described herein is indirectly operably connected to the C-terminus of the first immunomodulatory protein through a peptide linker; the N-terminus of a third immunomodulatory protein described herein is indirectly operably connected to the C-terminus of the other heavy chain of the full-length anti-integrin (e.g., α4β7 integrin) antibody through a peptide linker; and the N-terminus of a fourth immunomodulatory protein described herein is indirectly operably connected through a peptide linker to the C-terminus of the third immunomodulatory protein through a peptide linker. In some embodiments, the first and third immunomodulatory proteins have an identical amino acid sequence; and the second and fourth immunomodulatory proteins have an identical amino acid sequence.

FIG. 7C is a graphical representation of an exemplary format of an immunomodulatory fusion protein described herein, wherein the N-terminus of an Ig Fc region is indirectly operably connected to the C-terminus of an anti-integrin (e.g., α4β7 integrin) scFv through a peptide linker, and the N-terminus of an immunomodulatory protein is indirectly operably connected to the C-terminus of the Ig Fc region through a peptide linker.

FIG. 8 is a line graph showing the relative inhibition of the binding of integrin α4β7 to MAdCAM-1 in cell cultures treated with the indicated agent (IFP-13, IFP-14, or anti-integrin α4β7 antibody (positive control)) at the indicated concentration.

FIG. 9 is a is a line graph showing the % of TNFα activity in cell cultures treated with the indicated agent (IFP-13, IFP-14, or anti-integrin α4β7 antibody (positive control)) at the indicated concentration.

FIG. 10 is a is a line graph showing the NFκB activity (as a measure of TL1A inhibition) in cell cultures treated with the indicated agent (IFP-13, IFP-14, or anti-integrin α4β7 antibody (positive control)) at the indicated concentration.

FIG. 11 is a line graph showing the relative inhibition of the binding of integrin α4β7 to MAdCAM-1 in cell cultures treated with the indicated agent (IFP-12, negative control) at the indicated concentration.

FIG. 12 is a is a line graph showing the % of TNFα activity in cell cultures treated with the indicated agent (IFP-12, TNF Binder (positive control)) at the indicated concentration.

FIG. 13 is a line graph showing the relative inhibition of the binding of integrin α4β7 to MAdCAM-1 in cell cultures treated with the indicated agent (IFP-13, IFP-14, IFP-16, or anti-integrin α4β7 antibody (positive control)) at the indicated concentration.

FIG. 14 is a is a line graph showing the % of TNFα activity in cell cultures treated with the indicated agent (IFP-13, IFP-14, IFP-16, or anti-integrin α4β7 antibody (positive control)) at the indicated concentration.

FIG. 15 is a is a line graph showing the NFκB activity (as a measure of TL1A inhibition) in cell cultures treated with the indicated agent (IFP-13, IFP-14, IFP-16, or anti-integrin α4β7 antibody (positive control)) at the indicated concentration.

### 5. DETAILED DESCRIPTION

The inventors have, inter alia, developed immunomodulatory fusion proteins that, e.g., are capable of simultaneously binding and/or functionally targeting multiple proteins and/or signaling pathways. Accordingly, the novel immunomodulatory fusion proteins disclosed herein may be useful for various methods, including, e.g., selectively inhibiting or reducing (e.g., preventing) binding of a plurality of cognate receptor/ligand pairs, inhibiting or reducing (e.g., preventing) signaling mediated through the binding of such cognate receptor/ligand pairs, and modulating (e.g., suppressing or reducing (e.g., preventing)) an immune response. As such, the current disclosure provides, inter alia, novel immunomodulatory fusion proteins, nucleic acid molecules encoding the same, and pharmaceutical compositions comprising any of the foregoing, along with the methods for utilizing the same.

TABLE OF CONTENTS

| | |
|---|---|
| 5.1 | Definitions |
| 5.2 | Immunomodulatory Proteins and Fusion Proteins Thereof |
| 5.2.1 | Combinatorial Immunomodulatory Proteins |
| 5.2.1.1 | IL-10R Binding Proteins |
| 5.2.1.1(i) | Exemplary Properties of IL-10R Binding Proteins |
| 5.2.1.2 | TL1A Binding Proteins |
| 5.2.1.2(i) | Exemplary Properties of TL1A Binding Proteins |
| 5.2.1.3 | TNFα Binding Proteins |
| 5.2.1.3(i) | Exemplary Properties of TNFα Binding Proteins |
| 5.2.1.4 | CD30 Ligand Binding Proteins |
| 5.2.1.4(i) | Exemplary Properties of CD30L Binding Proteins |
| 5.2.2 | Integrin Targeting Immunomodulatory Fusion Proteins |
| 5.2.2.2 | Integrin Binding Domains |
| 5.2.2.3 | IL10-R Binding Proteins, TL1A Binding Proteins, TNFα |
| | Binding Proteins, and CD30L Binding Proteins |
| 5.3 | Half-Life Extension Moieties |
| 5.4 | Ig Fusion Proteins |
| 5.4.1 | Antibody Fusion Proteins |
| 5.4.2 | Ig Constant Region (e.g., Ig Fc) Fusion Proteins |
| 5.4.3 | Half-Life Extension |
| 5.4.4 | Ig Fc Effector Function |
| 5.4.4.1 | Reduced Ig Fc Effector Function |
| 5.4.4.2 | Enhanced Ig Effector Function |
| 5.5 | Signal Peptides |
| 5.6 | Components & Formats |
| 5.6.1 | Exemplary Combinations of Immunomodulatory |
| | Protein Components |
| 5.6.2 | Exemplary Formats |
| 5.6.3 | Multimeric Fusion Proteins |
| 5.7 | Exemplary Immunomodulatory Fusion Proteins |
| 5.8 | Exemplary Properties of Immunomodulatory |
| | Fusion Proteins |
| 5.9 | Conjugates & Additional Fusion Proteins |
| 5.9.1 | Radioligands |
| 5.9.2 | Chimeric Antigen Receptors |
| 5.10 | Methods of Making Proteins |
| 5.11 | Nucleic Acid Molecules |
| 5.11.1 | DNA Molecules |
| 5.11.2 | RNA Molecules |
| 5.12 | Vectors |
| 5.12.1 | Non-Viral Vectors |
| 5.12.2 | Viral Vectors |
| 5.13 | Cells |
| 5.14 | Carriers |
| 5.14.1 | Carriers of Immunomodulatory Fusion Proteins |
| 5.14.2 | Carriers Conjugated to Immunomodulatory Fusion |
| | Proteins |
| 5.14.3 | Lipid Based Carriers/Lipid Nanoformulations |
| 5.14.3.1 | Cationic Lipids (Positively Charged) and Ionizable |
| | Lipids |
| 5.14.3.2 | Non-Cationic Lipids (e.g., Phospholipids) |
| 5.14.3.3 | Structural Lipids |
| 5.14.3.4 | Polymers and Polyethylene Glycol (PEG) - Lipids |
| 5.14.3.5 | Percentages of Lipid Nanoformulation Components |
| 5.15 | Pharmaceutical Compositions |
| 5.16 | Combination Regimens |
| 5.17 | Combination Compositions |
| 5.18 | Methods of Use |
| 5.18.1 | Methods of Inhibiting or Reducing (e.g., Preventing) |
| | Receptor-Ligand Interactions |
| 5.18.2 | Methods of Inhibiting or Reducing (e.g., Preventing) |
| | Binding of a Plurality of Respective Receptor |
| | Ligand Interactions |
| 5.18.3 | Methods of Inhibiting or Reducing (e.g., Preventing) |
| | Signaling Mediated by a Receptor Ligand Interaction |
| 5.18.4 | Methods of Inhibiting or Reducing (e.g., Preventing) |
| | Signaling Mediated by a Plurality of Respective |
| | Receptor Ligand Interactions |
| 5.18.5 | Methods of Suppressing or Reducing (e.g., Preventing) |
| | a Pro-Inflammatory Immune Response |
| 5.18.6 | Methods of Preventing, Treating, or Ameliorating a |
| | Disease in a Subject in Need Thereof |
| 5.19 | Kits |

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

Use of the singular herein includes the plural unless specifically stated otherwise. For example, as used herein, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The term “about” refers to a value or composition that is within an acceptable error range for the particular value or composition as determined by one of ordinary skill in the art, which will depend in part on how the value or composition is measured or determined, i.e., the limitations of the measurement system. When particular values or compositions are provided herein, unless otherwise stated, the meaning of “about” should be assumed to be within an acceptable error range for that particular value or composition.

Where proteins and/or polypeptides are described herein, it is understood that nucleic acid molecules (e.g., RNA (e.g., mRNA) or DNA molecules) encoding the protein are also provided herein.

Where proteins, peptides, nucleic acid molecules, vectors, carriers, etc. are described herein, it is understood that isolated forms of the proteins, peptides, nucleic acid molecules, vectors, carriers, etc. are also provided herein.

Where proteins, peptides, nucleic acid molecules, etc. are described herein, it is understood that recombinant forms of the proteins, peptides, nucleic acid molecules, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa.

As used herein, the term “adjuvant” refers to a substance that causes stimulation of the immune system of a subject when administered to the subject.

As used herein, the term “administering” refers to the physical introduction of an agent, e.g., a therapeutic agent (or a precursor of the therapeutic agent that is metabolized or altered within the body of the subject to produce the therapeutic agent in vivo) or vaccine to a subject, using any of the various methods and delivery systems known to those skilled in the art. Administering can also be performed, for example, once, a plurality of times, and/or over one or more extended periods. The term administering includes both self-administration by the subject themselves and administration to the subject by another.

As used herein, the term “affinity” refers to the strength of the binding of one protein (e.g., a Ligand) to another protein (e.g., a Receptor). The affinity of a protein is measured by the dissociation constant Kd, defined as [Ligand]×[Receptor]/[Ligand-Receptor] where [Ligand-Receptor] is the molar concentration of the Ligand-Receptor complex, [Ligand] is the molar concentration of the unbound Ligand and [Receptor] is the molar concentration of the unbound Receptor. The affinity constant Ka is defined by 1/Kd. Standard methods of measuring affinity are known to the person of ordinary skill in the art and described herein, see, e.g., § 5.9.

As used herein, the term “agent” is used generically to describe any macro or micro molecule. Exemplary agents include, but are not limited proteins, peptides, nucleic acid molecules (e.g., DNA molecules, RNA molecules), vectors, carriers, carbohydrates, lipids, synthetic polymers, etc.

As used herein, the term “antibody” or “antibodies” is used in the broadest sense and encompasses various immunoglobulin (Ig) (e.g., human Ig (hIg), murine Ig (mIg)) structures, including, but not limited to monoclonal antibodies, polyclonal antibodies, multispecific (e.g., bispecific, trispecific) antibodies, and antibody fragments so long as they exhibit the desired antigen-binding activity (i.e., antigen binding fragments or variants). The term antibody thus includes, for example, full-length antibodies; antigen-binding fragments of full-length antibodies; molecules comprising antibody CDRs, VH regions, and/or VL regions; and antibody-like scaffolds (e.g., fibronectins). Examples of antibodies include, without limitation, monoclonal antibodies, polyclonal antibodies, monospecific antibodies, multispecific antibodies, human antibodies, humanized antibodies, chimeric antibodies, camelized antibodies, intrabodies, affybodies, diabodies, tribodies, heteroconjugate antibodies, antibody-drug conjugates, single domain antibodies (e.g., VHH, (VHH)₂), single chain antibodies, single-chain Fvs (scFv; (scFv)₂), Fab fragments (e.g., Fab, single chain Fab (scFab), F(ab′)₂ fragments, disulfide-linked Fvs (sdFv), Fc fusions (e.g., Fab-Fc, scFv-Fc, VHH-Fc, (scFv)₂-Fc, (VHH)₂-Fc), and antigen-binding fragments of any of the above, and conjugates or fusion proteins comprising any of the above. Antibodies can be of Ig isotype (e.g., IgG, IgE, IgM, IgD, or IgA), any class (e.g., IgG₁, IgG₂, IgG₃, IgG₄, IgA₁ or IgA₂), or any subclass (e.g., IgG₂ₐ or IgG_{2b}) of Ig). In certain embodiments, antibodies described herein are IgG antibodies, or a class (e.g., human IgG₁ or IgG₄) or subclass thereof. In certain embodiments, antibodies described herein are mIgG antibodies, or a class (e.g., mIgG1 or mIgG2a) or subclass thereof. In some embodiments, the antibody is a human, humanized, or chimeric IgG₁ or IgG₄ monoclonal antibody. In some embodiments, the term antibodies refers to a monoclonal or polyclonal antibody population. Antibodies described herein can be produced by any standard methods known in the art, e.g., recombinant production in host cells, see, e.g., § 5.11; or synthetic production.

As used herein, the term “antibody mimetic” refers to non-Ig based antigen binding domain. Various antibody-like scaffolds are known in the art. For example, 10th type III domain of fibronectin (e.g., AdNectins®) and designed ankyrin repeat proteins (e.g., DARPins®) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008), the full contents of each of which is incorporated by reference herein for all purposes. Exemplary antibody-like scaffolds include, but are not limited to, lipocalins (see, e.g., U.S. Pat. No. 7,250,297) (e.g., Anticalin®), protein A-derived molecules such as z-domains of protein a (see, e.g., U.S. Pat. No. 5,831,012) (e.g., Affibody®), A domains of membrane receptors stabilized by disulfide bonds and Ca2+ (see, e.g., U.S. Pat. No. 7,803,907) (e.g., Avimer/Maxibody®), a serum transferrin (see, e.g., US2004023334) (e.g., Transbody®); a designed ankyrin repeat protein (see, e.g., U.S. Pat. No. 7,417,130) (e.g., DARPin®), a fibronectin (see, e.g., U.S. Pat. No. 6,818,418) (e.g., AdNectin®), a C-type lectin domain (see, e.g., US2004132094) (e.g., Tetranectin®); a human gamma-crystallin or ubiquitin (see, e.g., U.S. Pat. No. 7,838,629) (e.g., Affilin®); a kunitz type domain of human protease inhibitors (see, e.g., US2004209243), C-Type Lectins (see, e.g., US2004132094) (e.g., Tetranectins®), cysteine knots or knottins (see, e.g., U.S. Pat. No. 7,186,524) (e.g., Microbodies®), nucleic acid aptamers (see, e.g., U.S. Pat. No. 5,475,096), thioredoxin A scaffold (see, e.g., U.S. Pat. No. 6,004,746) (peptide aptamers), and 10th type III domain of fibronectin (see, e.g., U.S. Pat. No. 6,818,418) (e.g., AdNectins®), and cystine-dense peptides (see, e.g., WO2023023031). Additional exemplary antibody-like scaffolds are known in the art and for example described in Storz U. Intellectual property protection: strategies for antibody inventions. MAbs. 2011; 3(3):310-317. doi:10.4161/mabs.3.3.15530. The entire contents of each of the foregoing references is incorporated herein by reference for all purposes. Antibody like scaffolds include e.g., naturally occurring antigen binders, variant (e.g., functional variants) of naturally occurring antigen binders, fragments (e.g., functional fragments) of naturally occurring antigen binders, and synthetic antigen binders (i.e., not naturally occurring antigen binders).

As used herein the term “CD30L” or “CD30 ligand” refers to the type II transmembrane protein of the TNFSF. The amino acid sequence of an exemplary reference membrane human CD30L (hCD30L) protein is set forth in SEQ ID NO: 31.

As used herein the term “CD30” refers to the type I transmembrane receptor of the TNFSF that binds CD30L. The amino acid sequence of an exemplary reference immature human CD30 (hCD30) protein is set forth in SEQ ID NO: 32 and the amino acid sequence of an exemplary reference mature hCD30 protein is set forth in SEQ ID NO: 32.

The terms “CH1” and “CH1 region” are used interchangeably herein and refer to the first constant region of an immunoglobulin heavy chain. The amino acid sequence of an exemplary reference hIgG1 CH1 region is set forth in SEQ ID NO: 632; and the amino acid sequence of an exemplary reference hIgG4 CH1 region is set forth in SEQ ID NO: 647.

The terms “CH2” and “CH2 region” are used interchangeably herein and refer to the second constant region of an immunoglobulin heavy chain. The amino acid sequence of an exemplary reference hIgG1 CH2 region is set forth in SEQ ID NO: 634; and the amino acid sequence of an exemplary reference hIgG4 CH2 region is set forth in SEQ ID NO: 650.

The terms “CH3” and “CH3 region” are used interchangeably herein and refer to the third constant region of an immunoglobulin heavy chain. The amino acid sequence of an exemplary reference hIgG1 CH3 region is set forth in SEQ ID NO: 635; and the amino acid sequence of an exemplary reference hIgG4 CH3 region is set forth in SEQ ID NO: 651.

As used herein, the term “circular RNA” refers to a translatable RNA molecule that forms a circular structure through covalent or non-covalent bonds. In some embodiments, the circular RNA is covalently closed.

As used herein, the term “conjugation” refers to chemical conjugation of a protein with a moiety (e.g., small molecule, polypeptide, nucleic acid molecule, carbohydrate, lipid, synthetic polymer (e.g., polymers of polyethylene glycol (PEG)), etc.). The moiety can be directly connected to the protein or indirectly connected through a linker, e.g., as described herein. Chemical conjugation methods are well known in the art, as are commercially available conjugation reagents and kits, with detailed instructions for their use readily available from the commercial suppliers.

As used herein the term “DcR3” or “Tumor Necrosis Factor Receptor Superfamily Member 6B” refers to the receptor of the TNFSF that binds, e.g., FASL, LIGHT. The amino acid sequence of an exemplary reference immature human DR3 (hDR3) protein is set forth in SEQ ID NO: 27 and the amino acid sequence of an exemplary reference mature hDR3 protein is set forth in SEQ ID NO: 28.

As used herein the term “DR3” or “Tumor Necrosis Factor Receptor Superfamily Member 25” refers to the receptor of the TNFSF that binds, e.g., TL1A. The amino acid sequence of an exemplary reference immature human DR3 (hDR3) protein is set forth in SEQ ID NO: 25 and the amino acid sequence of an exemplary reference mature hDR3 protein is set forth in SEQ ID NO: 26.

As used herein, the term “derived from,” with reference to a nucleic acid molecule refers to a nucleic acid molecule that has at least 70% sequence identity to a reference nucleic acid molecule (e.g., a naturally occurring nucleic acid molecule) or a fragment thereof. The term “derived from,” with reference to a protein refers to a protein that comprises an amino acid sequence that has at least 70% sequence identity to the amino acid sequence of a reference protein (e.g., a naturally occurring protein). The term “derived from” as used herein does not denote any specific process or method for obtaining the nucleic acid molecule, polypeptide, or protein. For example, the nucleic acid molecule, polypeptide, or protein can be recombinantly produced or chemically synthesized.

As used herein, the term “diagnosing” or “diagnosis” refers to a determination of the presence, absence, severity, or course of treatment of a disease (e.g., an infection, e.g., a viral infection). The term “diagnosing” encompasses an initial determination as well as subsequent determinations (e.g., monitoring) after the initial determination.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

The term “effector function” when used in reference to an antibody refers to those biological activities attributable to the Fc region of an antibody, which therefore vary with the antibody isotype. Antibody effector functions include, but are not limited to, antibody-dependent cell-mediated cytotoxicity (ADCC), antibody-dependent cellular phagocytosis (ADCP), complement dependent cytotoxicity (CDC), Fc receptor binding (e.g., FcγRI, FcγRIIa, FcγRIIc, FcγRIIIa, and/or FcγRIIIb (e.g., FcγRI, FcγRIIa, and/or FcγRIIIa)), and Clq binding.

As used herein the term “FAS” or “Tumor Necrosis Factor Receptor Superfamily Member 6” refers to the receptor of the TNFSF that binds, e.g., FASL. The amino acid sequence of an exemplary reference immature human FAS (hFAS) protein is set forth in SEQ ID NO: 23 and the amino acid sequence of an exemplary reference mature hFAS protein is set forth in SEQ ID NO: 24.

As used herein the term “FASL” or “FAS Ligand” or “Tumor Necrosis Factor Ligand Superfamily Member 6” refers to the immunomodulatory cytokine of the TNFSF. The amino acid sequence of an exemplary reference membrane human FASL (hFASL) protein is set forth in SEQ ID NO: 13 and the amino acid sequence of an exemplary reference soluble hFASL protein is set forth in SEQ ID NO: 14.

As used herein, the term “Fc region” refers to the C-terminal region of an Ig heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region, see, e.g., § 5.4.4. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated by reference herein for all purposes).

As used herein, the term “full-length antibody” refers to an antibody having a structure substantially similar to a native antibody structure. E.g., an antibody comprising (i) a first Ig light chain comprising from N- to C-terminus a light chain variable region (VL) region and a light chain constant region (CL) region; (ii) a first Ig heavy chain comprising from N- to C-terminus a heavy chain variable region (VH) region, a CH1 region, a hinge region, a CH2 region, and a CH3 region; (iii) a second Ig heavy chain comprising from N- to C-terminus a VH region, a CH1 region, a hinge region, a CH2 region, and a CH3 region; (iv) a second Ig light chain comprising from N- to C-terminus a VL region and a VH region; wherein said first light chain and said first heavy chain associate to form a first antigen binding domain; wherein said second light chain and said second heavy chain associate to form a second antigen binding domain; and wherein said first heavy chain and said second heavy chain associate to form a dimer. In some embodiments, the two heavy chains comprise a substantially identical amino acid sequence; and the two light chains comprise a substantially identical amino acid sequence. In some embodiments, the two heavy chains comprise a substantially identical amino acid sequence except for one or more amino acid modifications that promote heterodimerization of the correct heavy chains (e.g., as described herein); and the two light chains comprise a substantially identical amino acid sequence. Antibody chains may be substantially identical but not entirely identical if they differ due to post-translational modifications, such as C-terminal cleavage of lysine residues, C-terminal cleavage of glycine and lysine residues, alternative glycosylation patterns, etc.

The term “functional variant” as used herein in reference to a protein refers to a protein that comprises at least one but no more than 15%, not more than 12%, no more than 10%, no more than 8% amino acid variation (e.g., substitution, deletion, addition) compared to the amino acid sequence of a reference protein, wherein the protein retains at least one particular function of the reference protein. Not all functions of the reference protein (e.g., wild type) need be retained by the functional variant of the protein. In some instances, one or more functions are selectively reduced or eliminated. In some embodiments, the reference protein is a wild type protein. For example, a functional variant of a TL1A binding protein can refer to a TL1A binding protein comprising one or more amino acid substitution as compared to a reference TL1A (e.g., a wild type protein) that retains the ability to specifically bind TL1A.

The term “functional fragment” as used herein in reference to a protein refers to a fragment of a reference protein that retains at least one particular function. Not all functions of the reference protein need be retained by a functional fragment of the protein. In some instances, one or more functions are selectively reduced or eliminated. In some embodiments, the reference protein is a wild type protein. For example, a functional fragment of a TL1A binding protein can refer to a fragment of a TL1A binding protein that retains the ability to specifically bind TL1A.

As used herein, the term “fuse” and grammatical equivalents thereof refer to the operable connection of at least a first polypeptide to a second polypeptide, wherein the first and second polypeptides are not naturally found operably connected together. For example, the first and second polypeptides are derived from different proteins. The term fuse encompasses both a direct connection of the at least two polypeptides through a peptide bond, and the indirect connection through a linker (e.g., a peptide linker).

As used herein, the term “fusion protein” and grammatical equivalents thereof refers to a protein that comprises at least one polypeptide operably connected to another polypeptide, wherein the two polypeptides are not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “half-life extension moiety” refers to a moiety (e.g., small molecule, polypeptide, nucleic acid molecule, carbohydrate, lipid, synthetic polymer (e.g., polymers of PEG), etc.) that when conjugated or otherwise operably connected (e.g., fused) to a protein (the subject protein), increases the half-life of the subject protein in vivo when administered to a subject (e.g., a human subject). The pharmacokinetic properties of the protein can be evaluated utilizing in vivo models known in the art.

As used herein, the term “half-life extension polypeptide” or “half-life extension protein” refers to a protein that when operably connected to another protein (the subject protein), increases the half-life of the subject protein in vivo when administered to a subject (e.g., a human subject). The pharmacokinetic properties of the protein can be evaluated utilizing in vivo models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, nucleic acid molecule, carbohydrate, lipid, synthetic polymer (e.g., polymers of PEG), etc.) that is not joined to the polypeptide in nature.

As used, herein the term “heterologous signal peptide” refers to a signal peptide that is not operably connected to a subject protein in nature. For example, in reference to a polypeptide comprising a signal peptide from human IL-2 operably connected to human IL-12, the human IL-2 signal peptide would constitute a heterologous signal peptide. The terms “signal peptide” and “signal sequence” are used interchangeably herein.

The terms “hinge” or “hinge region” are used interchangeably herein and refer to the hinge region of an immunoglobulin heavy chain. The amino acid sequence of an exemplary reference hIgG1 hinge region is set forth in SEQ ID NO: 633; and the amino acid sequence of an exemplary reference hIgG4 hinge region is set forth in SEQ ID NO: 648 or 649.

As used herein, the term “heterologous signal peptide” refers to a signal peptide that is operably connected to a subject protein in nature. For example, in reference to a polypeptide comprising a signal peptide from human IL-2 operably connected to human IL-2, the human IL-2 signal peptide would constitute a heterologous signal peptide.

As used herein, the term “in combination with” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed. In some embodiments, administration of two or more agents or treatments in combination is such that the reduction in a symptom, or other parameter related to the condition is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, and intramuscular routes. The therapeutic agents can be administered by the same route or by different routes.

As used herein, the term “integrin” refers to a family of transmembrane cell-matrix adhesion proteins. Integrins are composed of two noncovalently associated transmembrane glycoprotein subunits called a and 3. A variety of human integrin heterodimers are formed from about 9 types of R subunits and about 24 types of a subunits. This diversity is further increased by alternative splicing of some integrin RNAs. See, e.g., Mezu-Ndubuisi O J, Maheshwari A. The role of integrins in inflammation and angiogenesis. Pediatr Res. 2021 May; 89(7):1619-1626. doi: 10.1038/s41390-020-01177-9. Epub 2020 Oct. 7. PMID: 33027803; PMCID: PMC8249239; Alberts B, Johnson A, Lewis J, et al. Molecular Biology of the Cell. 4th edition. New York: Garland Science; 2002. Integrins. Available from: https://www.ncbi.nlm.nih.gov/books/NBK26867/; Danen E H J. Integrins: An Overview of Structural and Functional Aspects. In: Madame Curie Bioscience Database [Internet]. Austin (TX): Landes Bioscience; 2000-2013. Available from: https://www.ncbi.nlm.nih.gov/books/NBK6259/; the entire contents of each of which are incorporated herein by reference for all purposes.

As used herein the term “α4β7 integrin,” and the like, refers to the integrin composed of the α4 and β7 subunits. The amino acid sequence of an exemplary reference mature human α4 polypeptide is set forth in SEQ ID NO: 578. The amino acid sequence of an exemplary reference mature human β7 polypeptide is set forth in SEQ ID NO: 579.

As used herein, the term “isolated” with reference to a polypeptide, protein, or nucleic acid molecule refers to a polypeptide, protein, or nucleic acid molecule that is substantially free of other cellular components with which it is associated in the natural state.

As used herein, the term “human interleukin 10” or “hIL-10” refers to the human immunomodulatory cytokine that mediates signaling through the human IL-10 Receptor. The amino acid sequence of an exemplary reference mature hIL-10 protein is set forth in SEQ ID NO: 2.

As used herein, the term “human IL-10 Receptor” or “hIL-10R” refers to the human heterodimeric cell surface complex comprised of hIL-10Rα and hIL-10Rβ, through which hIL-10 mediates signaling.

As used herein, the term “human IL-10 Receptor α” or “hIL-10Rα” refers to the alpha (α) subunit of the hIL-10 Receptor. The amino acid sequence of an exemplary reference mature hIL-10Rα polypeptide is set forth in SEQ ID NO: 4.

As used herein, the term “human IL-10 Receptor β” or “hIL-10Rβ” refers to the beta (p) subunit of the hIL-10 Receptor. The amino acid sequence of an exemplary reference mature hIL-10Rβ polypeptide is set forth in SEQ ID NO: 6.

As used herein the term “LIGHT” or “Tumor Necrosis Factor Ligand Superfamily Member 14” refers to the immunomodulatory cytokine of the TNFSF. The amino acid sequence of an exemplary reference membrane human LIGHT (hLIGHT) protein is set forth in SEQ ID NO: 11 and the amino acid sequence of an exemplary reference soluble hLIGHT protein is set forth in SEQ ID NO: 12.

As used herein the term “LIGHTR” or “Tumor Necrosis Factor Receptor Superfamily Member 14” refers to the receptor of the TNFSF that binds, e.g., LIGHT. The amino acid sequence of an exemplary reference immature human LIGHTR (hLIGHTR) protein is set forth in SEQ ID NO: 21 and the amino acid sequence of an exemplary reference mature hLIGHTR protein is set forth in SEQ ID NO: 22.

As used herein the term “LTR” or “Tumor Necrosis Factor Receptor Superfamily Member 3” refers to the receptor of the TNFSF that binds, e.g., LIGHT. The amino acid sequence of an exemplary reference immature human LTβR (hLTβR) protein is set forth in SEQ ID NO: 29 and the amino acid sequence of an exemplary reference mature hLTβR protein is set forth in SEQ ID NO: 30.

As used herein the term “LTα” or “Lymphotoxin a” or “Tumor Necrosis Factor Ligand Superfamily Member 1” refers to the multifunctional immunomodulatory cytokine of the TNFSF. The amino acid sequence of an exemplary reference immature form of human LTα (hTNFα) protein is set forth in SEQ ID NO: 9 and the amino acid sequence of an exemplary mature form of hLTα protein is set forth in SEQ ID NO: 10.

As used herein the term “MADCAM-1” or “mucosal vascular addressin cell adhesion molecule 1” refers to the endothelial cell adhesion molecule that interacts preferentially with specific integrins, including, e.g., α4β7 integrin. The amino acid sequence of an exemplary reference immature form of human MADCAM-1 (hMADCAM-1) is set forth in SEQ ID NO: 581; and the amino acid sequence of an exemplary mature form of hMADCAM-1 is set forth in SEQ ID NO: 582.

As used herein, the term “moiety” is used generically to describe any macro or micro molecule that can be operably connected to a protein described herein. Exemplary moieties include, but are not limited small molecules, polypeptides, nucleic acid molecules (e.g., DNA, RNA), carbohydrates, lipids, synthetic polymers (e.g., polymers of PEG).

As used herein, the term “modified nucleotide,” “nucleotide modification,” or use of the term “modification” and the like in reference to a nucleotide or nucleic acid sequence refers to a nucleotide comprising a chemical modification, e.g., a modified sugar moiety, a modified nucleobase, and/or a modified internucleoside linkage, or any combination thereof. Exemplary modifications are provided herein, see, e.g., § 5.12.2. In certain embodiments of the instant disclosure, inclusion of a deoxynucleotide—which is acknowledged as a naturally occurring form of nucleotide—if present within an RNA molecule is considered to constitute a modified nucleotide.

As used herein, the term “obtaining a sample” refers to the acquisition of a sample. The term includes the direct acquisition from a subject and the indirect acquisition through one or more third parties wherein one of the third parties directly acquired the sample from the subject.

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory nucleic acid molecule e.g., a promoter, enhancer, or other expression control element is operably linked to a nucleic acid molecule that encodes a protein if it affects the transcription of the nucleic acid molecule that encodes the protein. The term “operably connected” can also refer to the conjugation of a moiety to e.g., a nucleic acid molecule or polypeptide (e.g., the conjugation of a PEG polymer to a protein).

The determination of “percent identity” between two sequences (e.g., peptide or protein (amino acid sequences) or polynucleotide (nucleic acid sequences)) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences heterologous to a nucleic acid molecule described herein. BLAST protein searches can be performed with the XBLAST program parameters set, e.g., to score 50, wordlength=3 to obtain amino acid sequences heterologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” means a composition that is suitable for administration to an animal, e.g., a human subject, and comprises a therapeutic agent and a pharmaceutically acceptable carrier or diluent. A “pharmaceutically acceptable carrier or diluent” means a substance intended for use in contact with the tissues of human beings and/or non-human animals, and without excessive toxicity, irritation, allergic response, or other problem or complication, commensurate with a reasonable therapeutic benefit/risk ratio.

As used herein, the term “plurality” means 2 or more (e.g., 3 or more, 4 or more, 5 or more, 6 or more, 7 or more, 9 or more, or 10 or more).

As used herein, the term “poly(A) sequence,” refers to a sequence of adenosine nucleotides, typically located at the 3′-end of a coding linear RNA, of up to about 1000 adenosine nucleotides. In some embodiments, the poly(A) sequence is essentially homopolymeric, e.g., a poly(A) sequence of e.g., 100 adenosine nucleotides having essentially the length of 100 nucleotides. In other embodiments, the poly(A) sequence may be interrupted by at least one nucleotide different from an adenosine nucleotide, e.g., a poly(A) sequence of e.g., 100 adenosine nucleotides may have a length of more than 100 nucleotides (comprising 100 adenosine nucleotides and in addition said at least one nucleotide—or a stretch of nucleotides—different from an adenosine nucleotide). It has to be understood that “poly(A) sequence” as defined herein typically relates to mRNA—however in the context of the invention, the term likewise relates to corresponding sequences in a DNA molecule (e.g., a “poly(T) sequence”).

The terms “polynucleotide” and “nucleic acid molecule” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA molecules encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the terms “protein” and “polypeptide” refers to a polymer of at least 2 (e.g., at least 5) amino acids linked by a peptide bond. The term “polypeptide” does not denote a specific length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” and “protein” are used interchangeably herein. In some embodiments, the protein is folded into its three-dimensional structure. Where linear polypeptides are contemplated herein (i.e., primary structure (amino acid sequence)), it should be understood that proteins folded into their three-dimensional structure are also provided herein. Where proteins are contemplated herein (i.e., folded into their three-dimensional structure) polypeptides in their primary structure (i.e., the amino acid sequence) are also provided herein.

A “prophylactic” treatment is a treatment administered to a subject who does not exhibit signs of a disease or exhibits only early signs for the purpose of decreasing the risk of developing pathology.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester linkage found between the nucleotides of an unmodified nucleic acid molecule.

As used herein, the term “sample” encompass a variety of biological specimens obtained from a subject. Exemplary sample types include, e.g., blood and other liquid samples of biological origin (including, but not limited to, whole-blood, peripheral blood mononuclear cells (PBMCs), serum, plasma, urine, saliva, amniotic fluid, stool, synovial fluid, etc.), nasopharyngeal swabs, solid tissue samples such as biopsies (or cells derived therefrom and the progeny thereof), tissue cultures (or cells derived therefrom and the progeny thereof), and cell cultures (or cells derived therefrom and the progeny thereof). The term also includes samples that have been manipulated in any way after their procurement from a subject, such as by centrifugation, filtration, washing, precipitation, dialysis, chromatography, lysis, treatment with reagents, enriched for certain cell populations, refrigeration, freezing, staining, etc.

As used herein the term “TL1A” or “Tumor Necrosis Factor Ligand Superfamily Member 15” refers to the immunomodulatory cytokine of the TNFSF. The amino acid sequence of an exemplary reference membrane human TL1A (hTL1A) protein is set forth in SEQ ID NO: 15 and the amino acid sequence of an exemplary reference soluble hTL1A protein is set forth in SEQ ID NO: 16.

As used herein the term “TNFα” or “Tumor Necrosis Factor α” or “Tumor Necrosis Factor Ligand Superfamily Member 2” refers to the multifunctional immunomodulatory cytokine of the TNFSF. The amino acid sequence of an exemplary reference membrane human TNFα (hTNFα) protein is set forth in SEQ ID NO: 7 and the amino acid sequence of an exemplary reference soluble hTNFα protein is set forth in SEQ ID NO: 8.

As used herein the term “TNFR1” or “Tumor Necrosis Factor Receptor Superfamily Member 1A” refers to the receptor of the TNFSF that binds, e.g., TNFα. TNFR1 can be expressed both as a transmembrane protein and as a secreted protein through proteolytic processing. The amino acid sequence of an exemplary reference immature human TNFR1 (hTNFR1) protein is set forth in SEQ ID NO: 17 and the amino acid sequence of an exemplary reference mature hTNFR1 protein is set forth in SEQ ID NO: 18.

As used herein the term “TNFR2” or “Tumor Necrosis Factor Receptor Superfamily Member 1B” refers to the receptor of the TNFSF that binds, e.g., TNFα. The amino acid sequence of an exemplary reference immature human TNFR2 (hTNFR2) protein is set forth in SEQ ID NO: 19 and the amino acid sequence of an exemplary reference mature hTNFR2 protein is set forth in SEQ ID NO: 20.

As used herein, the term “translatable RNA” refers to any RNA that encodes at least one polypeptide and can be translated to produce the encoded protein in vitro, in vivo, in situ or ex vivo. A translatable RNA may be an mRNA or a circular RNA encoding a polypeptide.

The term “(scFv)₂” as used herein refers to an antibody that comprises a first and a second scFv operably connected (e.g., via a peptide linker). The first and second scFv can specifically bind the same or different antigens. In some embodiments, the first and second scFv are operably connected by a peptide linker.

The term “scFv-Fc” as used herein refers to an antibody that comprises a scFv operably linked (e.g., via a peptide linker) to an Fc domain or subunit of an Fc domain. In some embodiments, a scFv is operably connected to only a first Fc domain of a first and a second Fc domain pair. In some embodiments, a first scFv is operably connected to a first Fc domain and a second scFv is operably connected to a second Fc domain of a first and second Fc domain pair.

The term “(scFv)₂-Fc” as used herein refers to a (scFv)₂ operably linked (e.g., via a peptide linker) to an Fc domain or a subunit of an Fc domain. In some embodiments, a (scFv)₂ is operably connected to only a first Fc domain of a first and a second Fc domain pair. In some embodiments, a first (scFv)₂ is operably connected to a first Fc domain and a second (scFv)₂ is operably connected to a second Fc domain of a first and second Fc domain pair.

As used herein, the term “single domain antibody” or “sdAb” refers to an antibody having a single monomeric variable antibody domain. A sdAb is able to specifically bind to a specific antigen. A VHH (as defined herein) is an example of a sdAb.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal sequence peptide and the nucleic acid sequence encoding the signal peptide. Thus, references to a signal peptide in the context of a nucleic acid refers to the nucleic acid sequence encoding the signal peptide.

As used herein, the term “specifically binds” refers to preferential interaction, i.e., significantly higher binding affinity, between a first protein (e.g., a ligand) and a second protein (e.g., the ligand's cognate receptor) relative to other amino acid sequences. Herein, when a first protein is said to “specifically bind” to a second protein, it is understood that the first protein specifically binds to an epitope of the second protein. The term “epitope” refers to the portion of the second protein that the first protein specifically recognizes. The term specifically binds includes molecules that are cross reactive with the same epitope of a different species. For example, an antibody that specifically binds human CD30L may be cross reactive with CD30L of another species (e.g., cynomolgus, murine, etc.), and still be considered herein to specifically bind human CD30L. A protein can specifically bind more than one different protein.

As used herein, the term “subject” includes any animal, such as a human or other animal. In some embodiments, the subject is a vertebrate animal (e.g., mammal, bird, fish, reptile, or amphibian). In some embodiments, the subject is a human. In some embodiments, the method subject is a non-human mammal. In some embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In some embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

As used herein, the term “therapeutically effective amount” of a therapeutic agent refers to any amount of the therapeutic agent that, when used alone or in combination with another therapeutic agent, improves a disease condition, e.g., protects a subject against the onset of a disease (or infection); improves a symptom of disease or infection, e.g., decreases severity of disease or infection symptoms, decreases frequency or duration of disease or infection symptoms, increases disease or infection symptom-free periods; prevents or reduces impairment or disability due to the disease or infection; or promotes disease (or infection) regression. The ability of a therapeutic agent to improve a disease condition can be evaluated using a variety of methods known to the skilled practitioner, such as in human subjects during clinical trials, in animal model systems predictive of efficacy in humans, or by assaying the activity of the agent in in vitro assays.

As used herein, the terms “treat,” treating,” “treatment,” and the like refer to reducing or ameliorating a disease or infection and/or symptom(s) associated therewith or obtaining a desired pharmacologic and/or physiologic effect. It will be appreciated that, although not precluded, treating a disease or infection does not require that the disease or infection, or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease or infection. In some embodiments, the effect is preventative, i.e., the effect protects or prevents an occurrence or reoccurrence of a disease or infection. To this end, the presently disclosed methods comprise administering a therapeutically effective amount of a composition as described herein.

As used herein, the term “variant” or “variation” with reference to a nucleic acid molecule, refers to a nucleic acid molecule that comprises at least one substitution, alteration, inversion, addition, or deletion of nucleotide compared to a reference nucleic acid molecule. As used herein, the term “variant” or “variation” with reference to a protein refers to a protein that comprises at least one substitution, alteration, inversion, addition, or deletion of an amino acid residue compared to a reference protein.

As used herein, the term “variant Ig Fc fusion protein” refers to a fusion protein comprising an Ig Fc region, wherein the Ig Fc region comprises one or more variation (e.g., one or more amino acid substitution, deletion, or addition)) that decreases or abolishes one or more Fc effector function, relative to a reference Ig Fc fusion protein that does not comprise the one or more variation.

The terms “VL” and “VL domain” are used interchangeably to refer to the light chain variable region of an antibody.

The terms “VH” and “VH domain” are used interchangeably to refer to the heavy chain variable region of an antibody.

The term “VHH” as used herein refers to a type of single domain antibody (sdAb) that has a single monomeric heavy chain variable antibody domain (VH). Such antibodies can be found in or produced from camelid mammals (e.g., camels, llamas) which are naturally devoid of light chains or synthetically produced.

As used herein, the term “5′-untranslated region” or “5′-UTR” refers to a part of a nucleic acid molecule located 5′ (i.e., “upstream”) of a coding sequence and which is not translated into protein. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g., by enzymatic or post-transcriptional addition of a 5′-cap structure.

As used herein the term “3′-untranslated region” or “3′-UTR” refers to a part of a nucleic acid molecule located 3′ (i.e., downstream) of a coding sequence and which is not translated into protein. A 3′-UTR may located between a coding sequence and an (optional) terminal poly(A) sequence of a nucleic acid sequence. A 3′-UTR may comprise elements for controlling gene expression, also called regulatory elements. Such regulatory elements may be, e.g., ribosomal binding sites, miRNA binding sites etc.

### 5.2 Immunomodulatory Proteins and Fusion Proteins Thereof

### 5.2.1 Combinatorial Immunomodulatory Proteins

The present disclosure provides, inter alia, fusion proteins (e.g., immunomodulatory fusion proteins (IFPs)) (and functional fragments and variants thereof) that e.g., comprise a plurality of immunomodulatory proteins (and combination regimens comprising a plurality of immunomodulatory proteins or fusion proteins described herein). Immunomodulatory proteins described herein include, e.g., those described in §§ 5.2.1.1, 5.2.1.2, 5.2.1.3, and 5.2.1.4. Immunomodulatory proteins described herein include, e.g., those that specifically bind to the IL-10R (e.g., human IL-10R)) (see, e.g., § 5.2.1.1); those that specifically bind to TL1A (e.g., human TL1A)) (see, e.g., § 5.2.1.2); those that specifically bind to TNFα (e.g., human TNFα)) (see, e.g., § 5.2.1.3); and those that specifically bind to CD30L (e.g., human CD30L)) (see, e.g., § 5.2.1.4). Immunomodulatory proteins also include human proteins that specifically bind one (or more) of hIL-10R (e.g., hIL-10 (e.g., SEQ ID NO: 2)), hTL1A, hTNFα, or hCD30L.

The amino acid sequence of various reference proteins referred to throughout the instant disclosure is set forth Table 1 below.

TABLE 1
The Amino Acid Sequence of Human Reference Proteins.

| | | SEQ ID |
|---|---|---|
| Description | Amino Acid Sequence | NO |
| hIL-10 | MHSSALLCCLVLLTGVRASPGQGTQSENSCTHFPGNLPNMLRDLRD | 1 |
| (Immature - Signal | AFSRVKTFFQMKDQLDNLLLKESLLEDFKGYLGCQALSEMIQFYLE | |
| Peptide Underlined) | EVMPQAENQDPDIKAHVNSLGENLKTLRLRLRRCHRFLPCENKSKA | |
| | VEQVKNAFNKLQEKGIYKAMSEFDIFINYIEAYMTMKIRN | |
| hIL-10 | SPGQGTQSENSCTHFPGNLPNMLRDLRDAFSRVKTFFQMKDQLDNL | 2 |
| (Mature - No Signal | LLKESLLEDFKGYLGCQALSEMIQFYLEEVMPQAENQDPDIKAHVN | |
| Peptide) | SLGENLKTLRLRLRRCHRFLPCENKSKAVEQVKNAFNKLQEKGIYK | |
| | AMSEFDIFINYIEAYMTMKIRN | |
| hIL-10Rα | MLPCLVVLLAALLSLRLGSDAHGTELPSPPSVWFEAEFFHHILHWT | 3 |
| (Immature - Signal | PIPNQSESTCYEVALLRYGIESWNSISNCSQTLSYDLTAVTLDLYH | |
| Peptide Underlined) | SNGYRARVRAVDGSRHSNWTVTNTRFSVDEVTLTVGSVNLEIHNGF | |
| | ILGKIQLPRPKMAPANDTYESIFSHFREYEIAIRKVPGNFTFTHKK | |
| | VKHENFSLLTSGEVGEFCVQVKPSVASRSNKGMWSKEECISLTRQY | |
| | FTVTNVIIFFAFVLLLSGALAYCLALQLYVRRRKKLPSVLLFKKPS | |
| | PFIFISQRPSPETQDTIHPLDEEAFLKVSPELKNLDLHGSTDSGFG | |
| | STKPSLQTEEPQFLLPDPHPQADRTLGNREPPVLGDSCSSGSSNST | |
| | DSGICLQEPSLSPSTGPTWEQQVGSNSRGQDDSGIDLVQNSEGRAG | |
| | DTQGGSALGHHSPPEPEVPGEEDPAAVAFQGYLRQTRCAEKATKTG | |
| | CLEEESPLTDGLGPKFGRCLVDEAGLHPPALAKGYLKQDPLEMTLA | |
| | SSGAPTGQWNQPTEEWSLLALSSCSDLGISDWSFAHDLAPLGCVAA | |
| | PGGLLGSFNSDLVTLPLISSLQSSE | |
| hIL-10Rα | HGTELPSPPSVWFEAEFFHHILHWTPIPNQSESTCYEVALLRYGIE | 4 |
| (Mature - No Signal | SWNSISNCSQTLSYDLTAVTLDLYHSNGYRARVRAVDGSRHSNWTV | |
| Peptide) | TNTRFSVDEVTLTVGSVNLEIHNGFILGKIQLPRPKMAPANDTYES | |
| | IFSHFREYEIAIRKVPGNFTFTHKKVKHENFSLLTSGEVGEFCVQV | |
| | KPSVASRSNKGMWSKEECISLTRQYFTVTNVIIFFAFVLLLSGALA | |
| | YCLALQLYVRRRKKLPSVLLFKKPSPFIFISQRPSPETQDTIHPLD | |
| | EEAFLKVSPELKNLDLHGSTDSGFGSTKPSLQTEEPQFLLPDPHPQ | |
| | ADRTLGNREPPVLGDSCSSGSSNSTDSGICLQEPSLSPSTGPTWEQ | |
| | QVGSNSRGQDDSGIDLVQNSEGRAGDTQGGSALGHHSPPEPEVPGE | |
| | EDPAAVAFQGYLRQTRCAEKATKTGCLEEESPLTDGLGPKFGRCLV | |
| | DEAGLHPPALAKGYLKQDPLEMTLASSGAPTGQWNQPTEEWSLLAL | |
| | SSCSDLGISDWSFAHDLAPLGCVAAPGGLLGSFNSDLVTLPLISSL | |
| | QSSE | |
| hIL-10Rβ | MAWSLGSWLGGCLLVSALGMVPPPENVRMNSVNFKNILQWESPAFA | 5 |
| (Immature - Signal | KGNLTFTAQYLSYRIFQDKCMNTTLTECDFSSLSKYGDHTLRVRAE | |
| Peptide Underlined) | FADEHSDWVNITFCPVDDTIIGPPGMQVEVLADSLHMRFLAPKIEN | |
| | EYETWTMKNVYNSWTYNVQYWKNGTDEKFQITPQYDFEVLRNLEPW | |
| | TTYCVQVRGFLPDRNKAGEWSEPVCEQTTHDETVPSWMVAVILMAS | |
| | VFMVCLALLGCFALLWCVYKKTKYAFSPRNSLPQHLKEFLGHPHHN | |
| | TLLFFSFPLSDENDVFDKLSVIAEDSESGKQNPGDSCSLGTPPGQG | |
| | PQS | |
| hIL-10Rβ | MVPPPENVRMNSVNFKNILQWESPAFAKGNLTFTAQYLSYRIFQDK | 6 |
| (Mature - No Signal | CMNTTLTECDFSSLSKYGDHTLRVRAEFADEHSDWVNITFCPVDDT | |
| Peptide) | IIGPPGMQVEVLADSLHMRFLAPKIENEYETWTMKNVYNSWTYNVQ | |
| | YWKNGTDEKFQITPQYDFEVLRNLEPWTTYCVQVRGFLPDRNKAGE | |
| | WSEPVCEQTTHDETVPSWMVAVILMASVFMVCLALLGCFALLWCVY | |
| | KKTKYAFSPRNSLPQHLKEFLGHPHHNTLLFFSFPLSDENDVFDKL | |
| | SVIAEDSESGKQNPGDSCSLGTPPGQGPQS | |
| hTNFα Membrane | MSTESMIRDVELAEEALPKKTGGPQGSRRCLFLSLFSFLIVAGATT | 7 |
| Form | LFCLLHFGVIGPQREEFPRDLSLISPLAQAVRSSSRTPSDKPVAHV | |
| Uniprot ID: P01375 | VANPQAEGQLQWLNRRANALLANGVELRDNQLVVPSEGLYLIYSQV | |
| | LFKGQGCPSTHVLLTHTISRIAVSYQTKVNLLSAIKSPCQRETPEG | |
| | AEAKPWYEPIYLGGVFQLEKGDRLSAEINRPDYLDFAESGQVYFGI | |
| | IAL | |
| hTNFα Soluble | VRSSSRTPSDKPVAHVVANPQAEGQLQWLNRRANALLANGVELRDN | 8 |
| Form | QLVVPSEGLYLIYSQVLFKGQGCPSTHVLLTHTISRIAVSYQTKVN | |
| Uniprot ID: P01375 | LLSAIKSPCQRETPEGAEAKPWYEPIYLGGVFQLEKGDRLSAEINR | |
| | PDYLDFAESGQVYFGIIAL | |
| hLTα | MTPPERLFLPRVCGTTLHLLLLGLLLVLLPGAQGLPGVGLTPSAAQ | 9 |
| Immature Form | TARQHPKMHLAHSTLKPAAHLIGDPSKQNSLLWRANTDRAFLQDGF | |
| | SLSNNSLLVPTSGIYFVYSQVVFSGKAYSPKATSSPLYLAHEVQLF | |
| | SSQYPFHVPLLSSQKMVYPGLQEPWLHSMYHGAAFQLTQGDQLSTH | |
| | TDGIPHLVLSPSTVFFGAFAL | |
| hLTα | LPGVGLTPSAAQTARQHPKMHLAHSTLKPAAHLIGDPSKQNSLLWR | 10 |
| Mature Form | ANTDRAFLQDGFSLSNNSLLVPTSGIYFVYSQVVFSGKAYSPKATS | |
| | SPLYLAHEVQLFSSQYPFHVPLLSSQKMVYPGLQEPWLHSMYHGAA | |
| | FQLTQGDQLSTHTDGIPHLVLSPSTVFFGAFAL | |
| hLIGHT Membrane | MEESVVRPSVFVVDGQTDIPFTRLGRSHRRQSCSVARVGLGLLLLL | 11 |
| Form | MGAGLAVQGWFLLQLHWRLGEMVTRLPDGPAGSWEQLIQERRSHEV | |
| Uniprot ID: O43557 | NPAAHLTGANSSLTGSGGPLLWETQLGLAFLRGLSYHDGALVVTKA | |
| | GYYYIYSKVQLGGVGCPLGLASTITHGLYKRTPRYPEELELLVSQQ | |
| | SPCGRATSSSRVWWDSSFLGGVVHLEAGEKVVVRVLDERLVRLRDG | |
| | TRSYFGAFMV | |
| hLIGHT Soluble | LIQERRSHEVNPAAHLTGANSSLTGSGGPLLWETQLGLAFLRGLSY | 12 |
| Form | HDGALVVTKAGYYYIYSKVQLGGVGCPLGLASTITHGLYKRTPRYP | |
| Uniprot ID: O43557 | EELELLVSQQSPCGRATSSSRVWWDSSFLGGVVHLEAGEKVVVRVL | |
| | DERLVRLRDGTRSYFGAFMV | |
| hFASL Membrane | MQQPFNYPYPQIYWVDSSASSPWAPPGTVLPCPTSVPRRPGQRRPP | 13 |
| Form | PPPPPPPLPPPPPPPPLPPLPLPPLKKRGNHSTGLCLLVMFFMVLV | |
| Uniprot ID: P48023 | ALVGLGLGMFQLFHLQKELAELRESTSQMHTASSLEKQIGHPSPPP | |
| | EKKELRKVAHLTGKSNSRSMPLEWEDTYGIVLLSGVKYKKGGLVIN | |
| | ETGLYFVYSKVYFRGQSCNNLPLSHKVYMRNSKYPQDLVMMEGKMM | |
| | SYCTTGQMWARSSYLGAVFNLTSADHLYVNVSELSLVNFEESQTFF | |
| | GLYKL | |
| hFASL Soluble | QIGHPSPPPEKKELRKVAHLTGKSNSRSMPLEWEDTYGIVLLSGVK | 14 |
| Form | YKKGGLVINETGLYFVYSKVYFRGQSCNNLPLSHKVYMRNSKYPQD | |
| Uniprot ID: P48023 | LVMMEGKMMSYCTTGQMWARSSYLGAVFNLTSADHLYVNVSELSLV | |
| | NFEESQTFFGLYKL | |
| hTL1A Membrane | MAEDLGLSFGETASVEMLPEHGSCRPKARSSSARWALTCCLVLLPF | 15 |
| Form | LAGLTTYLLVSQLRAQGEACVQFQALKGQEFAPSHQQVYAPLRADG | |
| Uniprot ID: O95150 | DKPRAHLTVVRQTPTQHFKNQFPALHWEHELGLAFTKNRMNYTNKF | |
| | LLIPESGDYFIYSQVTFRGMTSECSEIRQAGRPNKPDSITVVITKV | |
| | TDSYPEPTQLLMGTKSVCEVGSNWFQPIYLGAMFSLQEGDKLMVNV | |
| | SDISLVDYTKEDKTFFGAFLL | |
| hTL1A Soluble | LKGQEFAPSHQQVYAPLRADGDKPRAHLTVVRQTPTQHFKNQFPAL | 16 |
| Form | HWEHELGLAFTKNRMNYTNKFLLIPESGDYFIYSQVTFRGMTSECS | |
| Uniprot ID: O95150 | EIRQAGRPNKPDSITVVITKVTDSYPEPTQLLMGTKSVCEVGSNWF | |
| | QPIYLGAMFSLQEGDKLMVNVSDISLVDYTKEDKTFFGAFLL | |
| hTNFR1 | MGLSTVPDLLLPLVLLELLVGIYPSGVIGLVPHLGDREKRDSVCPQ | 17 |
| Immature Form | GKYIHPQNNSICCTKCHKGTYLYNDCPGPGQDTDCRECESGSFTAS | |
| Uniprot ID: P19438 | ENHLRHCLSCSKCRKEMGQVEISSCTVDRDTVCGCRKNQYRHYWSE | |
| | NLFQCFNCSLCLNGTVHLSCQEKQNTVCTCHAGFFLRENECVSCSN | |
| | CKKSLECTKLCLPQIENVKGTEDSGTTVLLPLVIFFGLCLLSLLFI | |
| | GLMYRYQRWKSKLYSIVCGKSTPEKEGELEGTTTKPLAPNPSFSPT | |
| | PGFTPTLGFSPVPSSTFTSSSTYTPGDCPNFAAPRREVAPPYQGAD | |
| | PILATALASDPIPNPLQKWEDSAHKPQSLDTDDPATLYAVVENVPP | |
| | LRWKEFVRRLGLSDHEIDRLELQNGRCLREAQYSMLATWRRRTPRR | |
| | EATLELLGRVLRDMDLLGCLEDIEEALCGPAALPPAPSLLR | |
| hTNFR1 | LVPHLGDREKRDSVCPQGKYIHPQNNSICCTKCHKGTYLYNDCPGP | 18 |
| Mature Form | GQDTDCRECESGSFTASENHLRHCLSCSKCRKEMGQVEISSCTVDR | |
| Uniprot ID: P19438 | DTVCGCRKNQYRHYWSENLFQCFNCSLCLNGTVHLSCQEKQNTVCT | |
| | CHAGFFLRENECVSCSNCKKSLECTKLCLPQIENVKGTEDSGTTVL | |
| | LPLVIFFGLCLLSLLFIGLMYRYQRWKSKLYSIVCGKSTPEKEGEL | |
| | EGTTTKPLAPNPSFSPTPGFTPTLGFSPVPSSTFTSSSTYTPGDCP | |
| | NFAAPRREVAPPYQGADPILATALASDPIPNPLQKWEDSAHKPQSL | |
| | DTDDPATLYAVVENVPPLRWKEFVRRLGLSDHEIDRLELQNGRCLR | |
| | EAQYSMLATWRRRTPRREATLELLGRVLRDMDLLGCLEDIEEALCG | |
| | PAALPPAPSLLR | |
| hTNFR2 | MAPVAVWAALAVGLELWAAAHALPAQVAFTPYAPEPGSTCRLREYY | 19 |
| Immature Form | DQTAQMCCSKCSPGQHAKVFCTKTSDTVCDSCEDSTYTQLWNWVPE | |
| Uniprot ID: P20333 | CLSCGSRCSSDQVETQACTREQNRICTCRPGWYCALSKQEGCRLCA | |
| | PLRKCRPGFGVARPGTETSDVVCKPCAPGTFSNTTSSTDICRPHQI | |
| | CNVVAIPGNASMDAVCTSTSPTRSMAPGAVHLPQPVSTRSQHTQPT | |
| | PEPSTAPSTSFLLPMGPSPPAEGSTGDFALPVGLIVGVTALGLLII | |
| | GVVNCVIMTQVKKKPLCLQREAKVPHLPADKARGTQGPEQQHLLIT | |
| | APSSSSSSLESSASALDRRAPTRNQPQAPGVEASGAGEARASTGSS | |
| | DSSPGGHGTQVNVTCIVNVCSSSDHSSQCSSQASSTMGDTDSSPSE | |
| | SPKDEQVPFSKEECAFRSQLETPETLLGSTEEKPLPLGVPDAGMKP | |
| | S | |
| hTNFR2 | LPAQVAFTPYAPEPGSTCRLREYYDQTAQMCCSKCSPGQHAKVFCT | 20 |
| Mature Form | KTSDTVCDSCEDSTYTQLWNWVPECLSCGSRCSSDQVETQACTREQ | |
| Uniprot ID: P20333 | NRICTCRPGWYCALSKQEGCRLCAPLRKCRPGFGVARPGTETSDVV | |
| | CKPCAPGTFSNTTSSTDICRPHQICNVVAIPGNASMDAVCTSTSPT | |
| | RSMAPGAVHLPQPVSTRSQHTQPTPEPSTAPSTSFLLPMGPSPPAE | |
| | GSTGDFALPVGLIVGVTALGLLIIGVVNCVIMTQVKKKPLCLQREA | |
| | KVPHLPADKARGTQGPEQQHLLITAPSSSSSSLESSASALDRRAPT | |
| | RNQPQAPGVEASGAGEARASTGSSDSSPGGHGTQVNVTCIVNVCSS | |
| | SDHSSQCSSQASSTMGDTDSSPSESPKDEQVPFSKEECAFRSQLET | |
| | PETLLGSTEEKPLPLGVPDAGMKPS | |
| hLIGHTR | MEPPGDWGPPPWRSTPKTDVLRLVLYLTFLGAPCYAPALPSCKEDE | 21 |
| Immature Form | YPVGSECCPKCSPGYRVKEACGELTGTVCEPCPPGTYIAHLNGLSK | |
| Uniprot ID: Q92956 | CLQCQMCDPAMGLRASRNCSRTENAVCGCSPGHFCIVQDGDHCAAC | |
| | RAYATSSPGQRVQKGGTESQDTLCQNCPPGTFSPNGTLEECQHQTK | |
| | CSWLVTKAGAGTSSSHWVWWFLSGSLVIVIVCSTVGLIICVKRRKP | |
| | RGDVVKVIVSVQRKRQEAEGEATVIEALQAPPDVTTVAVEETIPSF | |
| | TGRSPNH | |
| hLIGHTR | LPSCKEDEYPVGSECCPKCSPGYRVKEACGELTGTVCEPCPPGTYI | 22 |
| Mature Form | AHLNGLSKCLQCQMCDPAMGLRASRNCSRTENAVCGCSPGHFCIVQ | |
| Uniprot ID: Q92956 | DGDHCAACRAYATSSPGQRVQKGGTESQDTLCQNCPPGTFSPNGTL | |
| | EECQHQTKCSWLVTKAGAGTSSSHWVWWFLSGSLVIVIVCSTVGLI | |
| | ICVKRRKPRGDVVKVIVSVQRKRQEAEGEATVIEALQAPPDVTTVA | |
| | VEETIPSFTGRSPNH | |
| hFAS | MLGIWTLLPLVLTSVARLSSKSVNAQVTDINSKGLELRKTVTTVET | 23 |
| Immature Form | QNLEGLHHDGQFCHKPCPPGERKARDCTVNGDEPDCVPCQEGKEYT | |
| Uniprot ID: P25445 | DKAHFSSKCRRCRLCDEGHGLEVEINCTRTQNTKCRCKPNFFCNST | |
| | VCEHCDPCTKCEHGIIKECTLTSNTKCKEEGSRSNLGWLCLLLLPI | |
| | PLIVWVKRKEVQKTCRKHRKENQGSHESPTLNPETVAINLSDVDLS | |
| | KYITTIAGVMTLSQVKGFVRKNGVNEAKIDEIKNDNVQDTAEQKVQ | |
| | LLRNWHQLHGKKEAYDTLIKDLKKANLCTLAEKIQTIILKDITSDS | |
| | ENSNFRNEIQSLV | |
| hFAS | QVTDINSKGLELRKTVTTVETQNLEGLHHDGQFCHKPCPPGERKAR | 24 |
| Mature Form | DCTVNGDEPDCVPCQEGKEYTDKAHFSSKCRRCRLCDEGHGLEVEI | |
| Uniprot ID: P25445 | NCTRTQNTKCRCKPNFFCNSTVCEHCDPCTKCEHGIIKECTLTSNT | |
| | KCKEEGSRSNLGWLCLLLLPIPLIVWVKRKEVQKTCRKHRKENQGS | |
| | HESPTLNPETVAINLSDVDLSKYITTIAGVMTLSQVKGFVRKNGVN | |
| | EAKIDEIKNDNVQDTAEQKVQLLRNWHQLHGKKEAYDTLIKDLKKA | |
| | NLCTLAEKIQTIILKDITSDSENSNFRNEIQSLV | |
| hDR3 | MEQRPRGCAAVAAALLLVLLGARAQGGTRSPRCDCAGDFHKKIGLF | 25 |
| Immature Form | CCRGCPAGHYLKAPCTEPCGNSTCLVCPQDTFLAWENHHNSECARC | |
| Uniprot ID: Q93038 | QACDEQASQVALENCSAVADTRCGCKPGWFVECQVSQCVSSSPFYC | |
| | QPCLDCGALHRHTRLLCSRRDTDCGTCLPGFYEHGDGCVSCPTSTL | |
| | GSCPERCAAVCGWRQMFWVQVLLAGLVVPLLLGATLTYTYRHCWPH | |
| | KPLVTADEAGMEALTPPPATHLSPLDSAHTLLAPPDSSEKICTVQL | |
| | VGNSWTPGYPETQEALCPQVTWSWDQLPSRALGPAAAPTLSPESPA | |
| | GSPAMMLQPGPQLYDVMDAVPARRWKEFVRTLGLREAEIEAVEVEI | |
| | GRFRDQQYEMLKRWRQQQPAGLGAVYAALERMGLDGCVEDLRSRLQ | |
| | RGP | |
| hDR3 | QGGTRSPRCDCAGDFHKKIGLFCCRGCPAGHYLKAPCTEPCGNSTC | 26 |
| Mature Form | LVCPQDTFLAWENHHNSECARCQACDEQASQVALENCSAVADTRCG | |
| Uniprot ID: Q93038 | CKPGWFVECQVSQCVSSSPFYCQPCLDCGALHRHTRLLCSRRDTDC | |
| | GTCLPGFYEHGDGCVSCPTSTLGSCPERCAAVCGWRQMFWVQVLLA | |
| | GLVVPLLLGATLTYTYRHCWPHKPLVTADEAGMEALTPPPATHLSP | |
| | LDSAHTLLAPPDSSEKICTVQLVGNSWTPGYPETQEALCPQVTWSW | |
| | DQLPSRALGPAAAPTLSPESPAGSPAMMLQPGPQLYDVMDAVPARR | |
| | WKEFVRTLGLREAEIEAVEVEIGRFRDQQYEMLKRWRQQQPAGLGA | |
| | VYAALERMGLDGCVEDLRSRLQRGP | |
| hDCR3 | MRALEGPGLSLLCLVLALPALLPVPAVRGVAETPTYPWRDAETGER | 27 |
| Immature Form | LVCAQCPPGTFVQRPCRRDSPTTCGPCPPRHYTQFWNYLERCRYCN | |
| Uniprot ID: O95407 | VLCGEREEEARACHATHNRACRCRTGFFAHAGFCLEHASCPPGAGV | |
| | IAPGTPSQNTQCQPCPPGTFSASSSSSEQCQPHRNCTALGLALNVP | |
| | GSSSHDTLCTSCTGFPLSTRVPGAEECERAVIDEVAFQDISIKRLQ | |
| | RLLQALEAPEGWGPTPRAGRAALQLKLRRRLTELLGAQDGALLVRL | |
| | LQALRVARMPGLERSVRERFLPVH | |
| hDCR3 | VAETPTYPWRDAETGERLVCAQCPPGTFVQRPCRRDSPTTCGPCPP | 28 |
| Mature Form | RHYTQFWNYLERCRYCNVLCGEREEEARACHATHNRACRCRTGFFA | |
| Uniprot ID: O95407 | HAGFCLEHASCPPGAGVIAPGTPSQNTQCQPCPPGTFSASSSSSEQ | |
| LTβR | CQPHRNCTALGLALNVPGSSSHDTLCTSCTGFPLSTRVPGAEECER | |
| | AVIDFVAFQDISIKRLQRLLQALEAPEGWGPTPRAGRAALQLKLRR | |
| | RLTELLGAQDGALLVRLLQALRVARMPGLERSVRERFLPVH | |
| hLTβR | MLLPWATSAPGLAWGPLVLGLFGLLAASQPQAVPPYASENQTCRDQ | 29 |
| Immature Form | EKEYYEPQHRICCSRCPPGTYVSAKCSRIRDTVCATCAENSYNEHW | |
| Uniprot ID: P36941 | NYLTICQLCRPCDPVMGLEEIAPCTSKRKTQCRCQPGMFCAAWALE | |
| | CTHCELLSDCPPGTEAELKDEVGKGNNHCVPCKAGHFQNTSSPSAR | |
| | CQPHTRCENQGLVEAAPGTAQSDTTCKNPLEPLPPEMSGTMLMLAV | |
| | LLPLAFFLLLATVFSCIWKSHPSLCRKLGSLLKRRPQGEGPNPVAG | |
| | SWEPPKAHPYFPDLVQPLLPISGDVSPVSTGLPAAPVLEAGVPQQQ | |
| | SPLDLTREPQLEPGEQSQVAHGTNGIHVTGGSMTITGNIYIYNGPV | |
| | LGGPPGPGDLPATPEPPYPIPEEGDPGPPGLSTPHQEDGKAWHLAE | |
| | TEHCGATPSNRGPRNQFITHD | |
| hLTβR | QAVPPYASENQTCRDQEKEYYEPQHRICCSRCPPGTYVSAKCSRIR | 30 |
| Mature Form | DTVCATCAENSYNEHWNYLTICQLCRPCDPVMGLEEIAPCTSKRKT | |
| Uniprot ID: P36941 | QCRCQPGMFCAAWALECTHCELLSDCPPGTEAELKDEVGKGNNHCV | |
| | PCKAGHFQNTSSPSARCQPHTRCENQGLVEAAPGTAQSDTTCKNPL | |
| | EPLPPEMSGTMLMLAVLLPLAFFLLLATVFSCIWKSHPSLCRKLGS | |
| | LLKRRPQGEGPNPVAGSWEPPKAHPYFPDLVQPLLPISGDVSPVST | |
| | GLPAAPVLEAGVPQQQSPLDLTREPQLEPGEQSQVAHGINGIHVTG | |
| | GSMTITGNIYIYNGPVLGGPPGPGDLPATPEPPYPIPEEGDPGPPG | |
| | LSTPHQEDGKAWHLAETEHCGATPSNRGPRNQFITHD | |
| hCD30L Membrane | MDPGLQQALNGMAPPGDTAMHVPAGSVASHLGTTSRSYFYLTTATL | 31 |
| Form | ALCLVFTVATIMVLVVQRTDSIPNSPDNVPLKGGNCSEDLLCILKR | |
| Uniprot ID: P32971 | APFKKSWAYLQVAKHLNKTKLSWNKDGILHGVRYQDGNLVIQFPGL | |
| | YFIICQLQFLVQCPNNSVDLKLELLINKHIKKQALVTVCESGMQTK | |
| | HVYQNLSQFLLDYLQVNTTISVNVDTFQYIDTSTFPLENVLSIFLY | |
| | SNSD | |
| hCD30 | MRVLLAALGLLFLGALRAFPQDRPFEDTCHGNPSHYYDKAVRRCCY | 32 |
| Immature Form | RCPMGLFPTQQCPQRPTDCRKQCEPDYYLDEADRCTACVTCSRDDL | |
| Uniprot ID: P28908 | VEKTPCAWNSSRVCECRPGMFCSTSAVNSCARCFFHSVCPAGMIVK | |
| | FPGTAQKNTVCEPASPGVSPACASPENCKEPSSGTIPQAKPTPVSP | |
| | ATSSASTMPVRGGTRLAQEAASKLTRAPDSPSSVGRPSSDPGLSPT | |
| | QPCPEGSGDCRKQCEPDYYLDEAGRCTACVSCSRDDLVEKTPCAWN | |
| | SSRTCECRPGMICATSATNSCARCVPYPICAAETVTKPQDMAEKDT | |
| | TFEAPPLGTQPDCNPTPENGEAPASTSPTQSLLVDSQASKTLPIPT | |
| | SAPVALSSTGKPVLDAGPVLFWVILVLVVVVGSSAFLLCHRRACRK | |
| | RIRQKLHLCYPVQTSQPKLELVDSRPRRSSTQLRSGASVTEPVAEE | |
| | RGLMSQPLMETCHSVGAAYLESLPLQDASPAGGPSSPRDLPEPRVS | |
| | TEHTNNKIEKIYIMKADTVIVGTVKAELPEGRGLAGPAEPELEEEL | |
| | EADHTPHYPEQETEPPLGSCSDVMLSVEEEGKEDPLPTAASGK | |
| hCD30 | FPQDRPFEDTCHGNPSHYYDKAVRRCCYRCPMGLFPTQQCPQRPTD | 33 |
| Mature Form | CRKQCEPDYYLDEADRCTACVTCSRDDLVEKTPCAWNSSRVCECRP | |
| Uniprot ID: P28908 | GMFCSTSAVNSCARCFFHSVCPAGMIVKFPGTAQKNTVCEPASPGV | |
| | SPACASPENCKEPSSGTIPQAKPTPVSPATSSASTMPVRGGTRLAQ | |
| | EAASKLTRAPDSPSSVGRPSSDPGLSPTQPCPEGSGDCRKQCEPDY | |
| | YLDEAGRCTACVSCSRDDLVEKTPCAWNSSRTCECRPGMICATSAT | |
| | NSCARCVPYPICAAETVTKPQDMAEKDTTFEAPPLGTQPDCNPTPE | |
| | NGEAPASTSPTQSLLVDSQASKTLPIPTSAPVALSSTGKPVLDAGP | |
| | VLFWVILVLVVVVGSSAFLLCHRRACRKRIRQKLHLCYPVQTSQPK | |
| | LELVDSRPRRSSTQLRSGASVTEPVAEERGLMSQPLMETCHSVGAA | |
| | YLESLPLQDASPAGGPSSPRDLPEPRVSTEHTNNKIEKIYIMKADT | |
| | VIVGTVKAELPEGRGLAGPAEPELEEELEADHTPHYPEQETEPPLG | |
| | SCSDVMLSVEEEGKEDPLPTAASGK | |

### 5.2.1.1 IL-10R Binding Proteins

In some aspects and embodiments, a fusion protein described herein (or a combination regimen described herein) comprises a protein that specifically binds the hIL-10R (e.g., hIL-110Rβ, hIL-10Rα).

The amino acid sequence of proteins capable of specifically binding the hIL-10R (e.g., hIL-10Rβ, hIL-10Rα) is set forth in Table 2 (SEQ ID NOS: 34-386). The amino acid sequence of the mature form of the proteins (i.e., lacking the native signal peptide) is set forth in SEQ ID NOS: 212-386. The amino acid sequence of the immature form of the proteins (i.e., containing the native signal peptide) is set forth in SEQ ID NOS: 34-211.

The signal peptides have been computationally predicted using standard methods (see, e.g., Teufel, F., Almagro Armenteros, J. J., Johansen, A. R. et al. SignalP 6.0 predicts all five types of signal peptides using protein language models. Nat Biotechnol (2022). https://doi.org/10.1038/s41587-021-01156-3 (hereinafter referred to as “Teufel 2022”), the entire contents of which is incorporated by reference herein for all purposes). A person of ordinary skill in the art would know how to experimentally identify and/or validate a computationally predicted signal peptide using standard methods known in the art, e.g., expression of the immunomodulatory protein from a host cell and sequencing of the intracellular form and the extracellular form of the expressed protein (see, e.g., Zhang Z, Henzel W J. Signal peptide prediction based on analysis of experimentally verified cleavage sites. Protein Sci. 2004; 13(10):2819-2824. doi:10.1110/ps.04682504 (hereinafter referred to as “Zhang 2004”), the entire contents of which is incorporated by reference herein for all purposes).

TABLE 2
The Amino Acid Sequence of IL-10R Binding Proteins.

| | | SEQ ID |
|---|---|---|
| Description | Amino Acid Sequence | NO |
| Immunomodulatory | MGKRAFVVSVAMALLGIYVITNTVNARHCMFGDSLRNSPDMKNML | 34 |
| Protein (IMP)-1 | QDLRGGYSGSGIKRTFQGKDTLDSMLLTQSLLDDEKGYLGCQALS | |
| with signal peptide | EMIQFYLEEVMPQAENHGPTDSVKQLGEKLHTLNQKFGECPRWEP | |
| | CYYNTTPAVENVKSVESKLQERGVYKAMSEFDIFINYIETYTTMK | |
| IMP-2 | MARRLTVASCGSVSLLAAFAAVLLIGCQLESGEALPLGSRSADSR | 35 |
| with signal peptide | SVDGQRVPAPQNNYPGLLRDLRLGYEGFKQKVTDSHPDETLLGSS | |
| | RLAGDLKGPLRCQALSEMIQFLLQVVLPDAENSRQDLRSQFSTLG | |
| | DRITGLRQQLRRDPTVFPCESRSDGVSDLRSAYTRLGSTGAEKVL | |
| | SEFDIFINYIEAYVTSV | |
| IMP-3 | MSNNKILVCAVIILTYTLYTDAYCVEYAESDEDRQQCSSSSNFPA | 36 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-4 | MANVVYVVLVISIMMANIHVSKTYCTSCSHHQCTEDENQKQDCED | 37 |
| with signal peptide | ANHSLPHMLRELRAAFGKVKTFFQMKDQLHSLLLTQSLLDDEKGY | |
| | LGCQALSEMIQFYLEEVMPQAENHGPEEHDNSLSEHGPDVKEHVN | |
| | SLGEKLKTLRLRLRRCHRFLPCENKSKAVEKVKRVESELQERGVY | |
| | KAMSEFDIFINYIETYMTT | |
| IMP-5 | MQGLQLLRGLLCCGVFAAASSRSPKNKPSIDCNPQTGDFVNMLKS | 38 |
| with signal peptide | MRQDYSRIRDTLHDRDKLHSSLLTGALLDEMMGYSGCRTTLLLME | |
| | HYLDTWYPAAYRHHLYDNQTLVVVDRMGSTLVALLKAMVQCPMLA | |
| | CGAPSPAMDKMLQQEAKMKKYTGVYKGISETDLLLGYLELYMMKE | |
| | KR | |
| IMP-6 | MRRRRSFGIVVSGAIRTLLMVAVVAVSVRGHEHKVPPACDPVHGN | 39 |
| with signal peptide | LAGIFKELRAIYASIREALQKKDTVYYTSLENDRVLQEMLSPMGC | |
| | RVTNELMEHYLDGVLPRAAHFDYDNSTLNGLHAFTSSMQALYQHM | |
| | LKCPALACTGKTPAWMYFLEVEHKLNPWRGTAKAAAEADLLLNYL | |
| | ETFLLQF | |
| IMP-7 | MGSRRLSRCSFATAVCLVAIVAAVAAKGRDSKPSPACDPMHGALA | 40 |
| with signal peptide | GIFKELRTTYRSVREALQTKDTVYYVSLFHEQLLQEMLSPVGCRV | |
| | TNELMQHYLDGVLPRAFHCGYDNATLNALHALSSSLSTLYQHMLK | |
| | CPALACTGQTPAWTQFLDTEHKLDPWKGTVKATAEMDLLLNYLET | |
| | FLLQS | |
| IMP-8 | MLSVMVSSSLVLIVFFLGASEEAKPATTTIKNTKPQCRPEDYATR | 41 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-9 | MALAHQLPVWIESIWILYFTLPLSEERVLPLRGNCKLLLQDTVIP | 42 |
| with signal peptide | NLLYSMRSIFQDIKPYFQGKDSLNNLLLSGQLLEDLQSPIGCDAL | |
| | SEMIQFYLEEVMPQAEIHHPKHKNSVMQLGETLHTLISQLQECTA | |
| | LFPCKHKSLGAQKIKEEVSKLGQYGIIKAVAEFDIFINYMESYFG | |
| | VK | |
| IMP-10 | MRRRRRSFGIIVAGAIGTLLMMAVVVLSAHDHEHKEVPPACDPVH | 43 |
| with signal peptide | GNLAGIFKELRATYASIREGLQKKDTVYYTSLENDRVLHEMLSPM | |
| | GCRVTNELMEHYLDGVLPRASHLDYDNSTLNGLHVFASSMQALYQ | |
| | HMLKCPALACTGKTPAWMYFLEVEHKLNPWRGTAKAAAEADLLLN | |
| | YLETELLQF | |
| IMP-11 | MRRRRSFGIVVAGAIGTLLMMAVVVFSAHEHKEVPPACDPVHGNL | 44 |
| with signal peptide | AGIFKELRATYASIREGLQKKDTVYYTSLENDRVLQEMLSPMGCR | |
| | VTNELMEHYLDGVLPRALHLDYDNSTLNGLHAFASSMQALYQHML | |
| | KCPALACTGKTPAWMYFLEVEHKLNPWRGTAKAAAEADLLLNYLE | |
| | TELLQF | |
| IMP-12 | MSKNKILVCVVIILTYTLYTDAYCVEYEESEEDRQQCSSSNEPAS | 45 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTKP | |
| IMP-13 | MSNNKILVCVVIILTYTLYTDAYCVEYEESEEDRQQCSSSNFPAS | 46 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTKT | |
| IMP-14 | MPNNKILVCAVIILTYTLYTDA | 47 |
| with signal peptide | YCVEYEESEEDRQQCSSSNFPASLPHMLRELRAAFGKVKTFFQMK | |
| | DQLNSMLLTQSLLDDFKGYLGCQALSEMIQFYLEEVMPQAENHGP | |
| | DIKEHVNSLGEKLKTLRLRLRRCHRFLPCENKSKAVEQVKRVENM | |
| | LQERGVYKAMSEFDIFINYIESYMTTKS | |
| IMP-15 | MSNKKILVCVVIILTYTLYTDAYCVEYKESEEDRQQCSSSSFPAS | 48 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTK | |
| IMP-16 | MSNNKILVCVAIILTYTLYTDAYCVEYAESDEDKQQCSGSNEPAS | 49 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTKM | |
| IMP-17 | MSKNKVLVCFVIILTYTLYTDAYCVEYEESEEDKQQCGSNGGPAS | 50 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTKM | |
| IMP-18 | MSNNKILVCAVIILTYTLYTDAYCVEYAESDEDRQQCSGSNEPAS | 51 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTKM | |
| IMP-19 | MSNNKILLCVAIILTYTLYTDAYCVEYEESEEDKQQCSSSSNFPA | 52 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-20 | MSNNKILVCAVIILTYTLYTDAYCIQYEESEEDKQQCSSSNFPAS | 53 |
| with signal peptide | LPHMLRELRAAFGKVKTFFQMKDQLNSMLLTQSLLDDEKGYLGCQ | |
| | ALSEMIRFYLEEVMPQAENHGPDIKEHVNSLGEKLKTLRLRLRRC | |
| | HRFLPCENKSKAVEQVKRVENMLQERGVYKAMSEFDIFINYIESY | |
| | MTTKM | |
| IMP-21 | MSKNKFLVCVVIILTYTLYTDAYCVEYEESEEDRQQCSSSNFPAS | 54 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLREKLKTLRLRLRRC | |
| | HRFLPCENKSKAVEQVKRVENMLQERGVYKAMSEFDIFINYIESY | |
| | MTTKT | |
| IMP-22 | MSKNKILVCFVIILTYTLYTDAYCVEYEESEEDKQQCGSSSNFPA | 55 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-23 | MSNNKILVCVAIILTYTLYTDAYCVEYAESDEDKQQCSGSNEPAS | 56 |
| with signal peptide | LPHMLRELRAAFGKVKTFFQMKDQLNSMLLTQSLLDDEKGYLGCQ | |
| | ALSEMIQFYLEEVMPRAENHGPDIKEHVNSLGEKLKTLRLRLRRC | |
| | HRFLPCENKSKAVEQVKRVENMLQERGVYKAMSEFDIFINYIESY | |
| | MTTKM | |
| IMP-24 | MSNNKILVCVVIILTYTLYTDAYCVEYEESEEDRQQCSGSSNFPA | 57 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-25 | MSNNKILVCAVIILTYTLYTDAYCVEYEESDEDRQQCSSSSNFPA | 58 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-26 | MSKNKILVCVAIILTYTLYTDAYCVEYEESDEDKQQCSSSTGAPA | 59 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-27 | MSKNKILVCVAIILTYTLYTDAYCVEYEETKEDEQQCSSSSNFPA | 60 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-28 | MSKNKILVCVVIILTYTLYTDAYCVEYEESEEDRQQCSSSNFPAS | 61 |
| with signal peptide | LPHMLRELRAAFGKVKTFFQMKDQLNSMLLTQSLLDDEKGYLGCQ | |
| | ALSEMIQFYLEEVMPQAENHGPDIKEHVNSLGEKLKTLRLRLRRC | |
| | HRFLPCENKSKAVEQVKRVENMLQERGDYKAMSEFDIFINYIESY | |
| | MTTKS | |
| IMP-29 | MSNNKILVCVVIILTYTLYTDAYCVEYEESEEDRQQCSSSSNFPA | 62 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRELPCEDKSKAVEQVKRVENMLQERGVYKAMSEFDILINYIES | |
| | YMTTKM | |
| IMP-30 | MSNNKILVCAVIILTYTLYTDAYCVEYEESDEDRQQCSSSSNFPA | 63 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMATKM | |
| IMP-31 | MFRALLLCCLALLAGVWADNRYDGQDGNDCPTLPTSLPHMLHELR | 64 |
| with signal peptide | AAFSRVKTFFQMKDQLDNMLLDGSLLEDFKGYLGCQALSEMIQFY | |
| | LEEVMPQAENHSTDQEKDKVNSLGEKLKTLRVRLRRCHRELPCEN | |
| | KSKAVEQVKSAFSKLQEKGVYKAMSEFDIFINYIEAYMTTKMKN | |
| IMP-32 | MSKNKILVCVAIILTYTLYTDAYCVEYLESREDEQQCSSSSNFPA | 65 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-33 | MSKNKILVCVAIILTYTLYTDAYCVEYEESKEDEQQCSGSNGASA | 66 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-34 | MSKNKILVCVAIILTYTLYTDAYCVEYLESGEDEQQCGSSSNFPA | 67 |
| with signal peptide | SLPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-35 | MSKNKILVCVAIILTYTLYTDAYCVEYLESREDEQQCSGSNGASA | 68 |
| with signal peptide | SLPHMLRELRAAFGKVKTFFQMKDQLNSMLLTQSLLDDEKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-36 | MPGAALLYCLFFVTGVWAESENNCTHFPTSLPHMLHELRAAFSRV | 69 |
| with signal peptide | KTFFQMKDQLDNMLLNGSLLEDFKGYLGCQALSEMIQFYLEEVMP | |
| | QAENHSGGGGPDIKEHVNSLGEKLKTLRVRLRRCHRFLPCENKSK | |
| | AVEQVKSAFSKLQEKGVYKAMSEFDIFINYIEAYMTTKMKNKK | |
| IMP-37 | MSNNKILVCVAIILTYTLYTDAYCVEYLESDEDKQHCSSSNGASA | 70 |
| with signal peptide | SSPHMLRELRAAFGKVKTFFQMKDQLNSMLLTQSLLDDFKGYLGC | |
| | QALSEMIQFYLEEVMPQAENHGPDIKEHVNSLGEKLKTLRLRLRR | |
| | CHRFLPCENKSKAVEQVKRVENMLQERGVYKAMSEFDIFINYIES | |
| | YMTTKM | |
| IMP-38 | MSNNKILVCVAIILTYTLYTNAYCVEYLESEEDKQQCGSNGASSS | 71 |
| with signal peptide | SPHMLRELRAAFGKVKTFFQMKDQLNSMLLTQSLLDDEKGYLGCQ | |
| | ALSEMIQFYLEEVMPQAENHGPDVKEHVNSLAEKLKTLRLRLRRC | |
| | HRFLPCENKSKAVEQVKRVENMLQERGVYKAMSEFDIFINYIESY | |
| | MTTKS | |
| IMP-39 | MSNNKILVCVAIILTYTLYTNAYCVEYLESEEDKQQCGSNGASSS | 72 |
| with signal peptide | SPHMLRELRAAFGKVKTFFQMKDQLNSMLLTQSLLDDEKGYLGCQ | |
| | ALSEMIQFYLEEVMPQAENHGPDVKEHVNSLGEKLKTLRLRLRRC | |
| | HRFLPCENKSKAVAQVKRVENMLQERGVYKAMSEFDIFINYIESY | |
| | MTTKS | |
| IMP-40 | ILVCFVIILTYTLYTDAYCVEYEESEEDRQQCSSSNFPASLPHML | 73 |
| with signal peptide | RELRAAFGKVKTFFQMKDQLNSMLLTQSLLDDEKGYLGCQALSEM | |
| | IQFYLEEVMPQAENHGPDIKEHVNSLGEKLKTLRLRLRRCHRFLP | |
| | CENKSKAVEQVKRVENMLQERGVYKAMSEFDIFINYIES | |
| IMP-41 | ILVCFVIILTYTLYTDAYCVEYEESEEDRQQCSSSNFPASLPHML | 74 |
| with signal peptide | RELRAAFGKVKTFFQMKDQLNSMLLTQSLLDDFKGYLGCQAFSEM | |
| | IQFYLEEVMPQAENHGPDIKEHVNSLGEKLKTLRLRLRRCHRFLP | |
| | CENKSKAVEQVKRVENMLQERGVYKAMSEFDIFINYIES | |
| IMP-42 | LVCVAIILTYTLYTDAYCVEYLESREDEQQCSSSSNEPASLPHML | 75 |
| with signal peptide | RELRAAFGKVKTFFQMKDQLNSMLLTQSLLDDEKGYLGCQALSEM | |
| | IQFYLEEVMPQAENHGPDIKEHVNSLGEKLKTLRLRLRRCHRFLP | |
| | CENKSKAVEQVKRVENMLQERGVYKAMSEFDIFINYIES | |
| IMP-43 | MGLRSGLTLQCLVILQCLVMLYLAPACKGVSNCGNLPHMLRDLRD | 76 |
| with signal peptide | AFSRVKTFFQMKDQLDNILLKESLLEDFKGYLGCQALSEMIQFYL | |
| | EEVMPQAENQDPHAKEHVNSLGENLKTLRLRLRRCHRFLPCENKS | |
| | KAVEQVKNAFSKLQEKGVYKAMSEFDIFINYIEAYMTMKIRR | |
| IMP-44 | LVCVAIILTYTLYTDAYCVEYLESREDEQQCGSSSNFPASLPHML | 77 |
| with signal peptide | RELRAAFGKVKTFFQMKDQLNSMLLTQSLLDDEKGYLGCQALSEM | |
| | IQFYLEEVMPQAENHGPDIKEHVNSLGEKLKTLRLRLRRCHRFLP | |
| | CENKSKAVEQVKRVENMLQERGVYKAMSEFDIFINYIES | |
| IMP-45 | ILVCFVIILTYTLYTDAYCVEYEESEEDRQQCSSSNFPASLPHML | 78 |
| with signal peptide | RELRAAFGKVKTFFQMKDQLNSMLLTQSLLDDEKGYLGCQALSEM | |
| | IQFYLEEVMPQAENHGPDIKEHVNSPGEKLKTLRLRLRRCHRELP | |
| | CENKSKAVEQVKRVENMLQERGVYKAMSEFDIFINYIES | |
| IMP-46 | MANVIYVVLALNILLSQIHVSNPYCTSCSYRDCTEDEDQKQQCEG | 79 |
| with signal peptide | GLRSLPHMLRELRAAFGKVKTFFQMKDQLHSLLLTQSLLDDEKGY | |
| | LGCQALSEMIQFYLEEVMPQAENHGPDIKEHVNSLGEKLKTLRLR | |
| | LRRCHRFLPCENKSKAVEKVKRVESELQERGVYKAMSEFDIFINY | |
| | IETYMT | |
| IMP-47 | ILVCFVIILTYTLYTDAYCVEYEESEEDRQQCSSSNFPASLPHMP | 80 |
| with signal peptide | RELRAAFGKVKTFFQMKDQLNSMLLTQSLLDDEKGYLGCQALSEM | |
| | IQFYLEEVMPQAENHGPDIKEHVNSLGEKLKTLRLRLRRCHRFLP | |
| | CENKSKAVEQVKRVENMLQERGVYKAMSEFDIFINYIES | |
| IMP-48 | MANVIYVVLALNILLSQFHVSNPYCTSCSHRDCTEDDEQKQQCEG | 81 |
| with signal peptide | GSGGLGSLPHMLRELRAAFGKVKTFFQMKDQLHSLLLTQSLLDDE | |
| | KGYLGCQALSEMIQFYLEEVMPQAENHGPDIKEHVNSLGEKLKTL | |
| | RLRLRRCHRFLPCENKSKAVEKVKRVESELQERGVYKAMSEFDIF | |
| | INYIETYMT | |
| IMP-49 | MERRLVVTLQCLVLLYLAPECGGTDQCDNFPQMLRDLRDAFSRVK | 82 |
| with signal peptide | TFFQTKDEVDNLLLKESLLEDFKGYLGCQALSEMIQFYLEEVMPQ | |
| | AENQDPGAKDHVNSLGENLKTLRLRLRRCHRFLPCENKSKAVEQI | |
| | KNAFNKLQEKGIYKAMSEFDIFINYIEAYMTIKAR | |
| IMP-50 | MERRLVVTLQCLVLLYLAPECGSTDQCDNFPQMLRDLRDAFSRVK | 83 |
| with signal peptide | TFFQTKDEVDNLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-51 | MERRLVVTLQCLVLLYLAPECGGTDQCDNFPQMLRDLRDAFSRVK | 84 |
| with signal peptide | TFFQTKDEVDNLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTMKAR | |
| IMP-52 | MGPRAGLALQCLLLLYLAPACKGVSNCGNLPHMLRDLRDAFSRVK | 85 |
| with signal peptide | TFFQMKDQLDNILLKESLLEDFKGYLGCQALSEMIQFYLEEVMPQ | |
| | AENQDPNAKEHVNSLGENLKTLRLRLRRCHRFLPCENKSKAVEQV | |
| | KNAFSKLQEKGVYKAMSEFDIFINYIEAYMTMKTRR | |
| IMP-53 | MERRLVVTLQCLVLLYLAPECGGTDQCDNFPQMLRDLRDAFSRVK | 86 |
| with signal peptide | TFFQTKDEVDNLLLKESLLEDFKGYLGCQALSEMIQFYLEEVMPQ | |
| | AENQDPEAKDHVNSLGENLKTIRLRLRRCHRFLPCENKSKAVEQI | |
| | KNAFNKLQEKGIYKAMSEFDIFINYIEAYMTIKAR | |
| IMP-54 | MERRLVVTLQCLVLLYLAPECGGTDQCDNFPQMLRDLRDAFSRVK | 87 |
| with signal peptide | TFFQTKDEVDSLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-55 | MERRLMVTLQCLVLLYLAPECGSTDQCDNFPQMLRDLRDAFSRVK | 88 |
| with signal peptide | TFFQTKDEVDNILLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-56 | MERRLVVTLQCLVLLYLAPECGGTDQCDNFPQMLRDLRDAFSRVK | 89 |
| with signal peptide | TFFQTKDEVDNLELKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-57 | MERRLVVTLQCLVLLYLAPECGGTDQCDNFPQMLRDLRDAFSRVK | 90 |
| with signal peptide | TFFQTKDEVDNLLLKESLLEDFKGYLGCQALSEMIQFYLEKVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-58 | MERRLMVTLQCLVLLYLAPECGSTDQCDNFPQMLRDLRDAFSRVK | 91 |
| with signal peptide | TFFQTKDAVDNLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFDIFINYIEAYMTIKAR | |
| IMP-59 | MELRSGLTLQCLVMLQCLVMLYLAPACKG | 92 |
| with signal peptide | ASNCGNLPHMLRDLRDAFSRVKTFFQMKDQLDNILLKESLLEDER | |
| | GYLGCQALSEMIQFYLEEVMPQAENQDPHSKEHVNSLGENLKTLR | |
| | LRLRRCHRFLPCENKGKAVEQVKNAFSKLQEKGVYKAMSEFDIFI | |
| | NYIEAYMTMKLRR | |
| IMP-60 | MERRLVVTLQCLVLLYLAPECGGTDQCDNFPQMLRDLRDAFSRVK | 93 |
| with signal peptide | TFFQTKDEVDNLLLKESLLEDFKGYLGCQALSEMIQFYLEEVMPQ | |
| | AENQDPEAKDHVNSLGENLKTLRLRLRRCHRFLPCENKSKAVEQI | |
| | KNAFNKLQEKGIYKAMSEFHIFINYIEAYMTIKAR | |
| IMP-61 | MERRLVVTLQCLVLLYLAPECGEMLRDLRDAFSRVKTFFQTKDEV | 94 |
| with signal peptide | DNLLLKESLLEDFKGYLGCQALSEMIQFYLEEVMPQAENQDPEAK | |
| | DHVNSLGENLKTLRLRLRRCHRFLPCENKSKAVEQIKNAFNKLQE | |
| | KGIYKAMSEFDIFINYIEAYMTIKAR | |
| IMP-62 | MELSLGLTLHFLVELCLAPACGRAETCGNIPHMLRDLRDAFSRVK | 95 |
| with signal peptide | TFFQMKDQLDNILLKESLLEDFKGYLGCQALSEMIQFYLEEVMPQ | |
| | AEAMSLKSQEHVNFLGENLNTLRLRLRRCHRFLPCENKSKAVEQV | |
| | KNAFSKLQEKGVYKAMSEFDIFINYIEAYMTMKLRR | |
| IMP-63 | MAYGKKIVAASLLVIPAYVVETNATANNRAQKCFCEDGSNAGNSE | 96 |
| with signal peptide | ETNTAAFQKKCDSEIPESLPYMLRDLRNSSVQTRRYFQEKDEENS | |
| | PLLTQKLLEDFKGYLGCQALSEMIQFYLEEVMPQAEDSNPSAKDS | |
| | VTSLGEKLKTLRLRLRRCHRFLPCENKSKAVENLKSKFGDLGNQG | |
| | VHKAMSEFDIFINYIETYMTTKMK | |
| IMP-64 | MLSVMVSSSLVLIVFELGASEEAKPATTTTIKNTKPQCRPEDYAT | 97 |
| with signal peptide | RLQDLRVTFHRVKPTLQREDDYSVWLDGTVVKGCWGCSVMDWLLR | |
| | RYLETVFPAGDHVYPGLKTELHSMRSTLESIYKDMRQCPLLGCGD | |
| | KSVISRLSQEAERKSDNGTRKGLSELDTLFSRLEEYLHSRK | |
| IMP-65 | MLSVMVSSSLVLIVFELGASEEAKPATTTTIKNTKPQCRPEDYAT | 98 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-66 | MLSVMVSSSLVLIVFFLGASEEAKPATTTTKNTKPQCRPEDYATR | 99 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-67 | MLSVMVSSSLVLIVFELGASEEAKPATTTTIKNTKPQCRPEDYAT | 100 |
| with signal peptide | RLQDLRVTFHRVKPTLQREDDYSVWLDGTMVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-68 | MLSVMVSSSLVLIVFFLGASEEAKPATTTTIKNTKPQCRPEDYAT | 101 |
| with signal peptide | RLQDLRVTFHRVKSTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-69 | MLSVMVSSSLVLIVFELGASEEAKPATTTTIKNTKPQCRPEDYAT | 102 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDMLFSRLEEYLHSRK | |
| IMP-70 | MLSVMVSSSLVLIVFELGASEEAKPATTTTIKNTKPQCRPEDYAT | 103 |
| with signal peptide | RLQDLRVTFHRVKPTLQREDDYSVWLDGTVVKGCWGCSVMDWLLR | |
| | RYLEIVLPAGDHVYPGLKTELHSMRSTLESIYKDMRQCPLLGCGD | |
| | KSVISRLSQEAERKSDNGTRKGLSELDTLFSRLEEYLHSRK | |
| IMP-71 | MLSVMVSSSLVLIVFFLGASEEAKPAATTTIKNTKPQCRPEDYAT | 104 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-72 | MLSVMVSSSLVLIVFFLGASEEAKPATTTTINNTKPQCRPEDYAT | 105 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-73 | MLSVMVSSSLVLIVFELGASEEAKPATTTTIKNTKPQCRPEDYAT | 106 |
| with signal peptide | RLQDLRVTFHRVKPTLQREDDYSVWLDGTVVKGCWGCSVMDWLLR | |
| | RYLEIVFPAGDHVYPGLKTELHSMHSTLESIYKDMRQCPLLGCGD | |
| | KSVISRLSQEAERKSDNGTRKGLSELDTLFSRLEEYLHSRK | |
| IMP-74 | MLSVMVSSSLVLIIFFLGASEEAKPATTTTIKNTKPQCRPEDYAT | 107 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGMRKGLSELDTLFSRLEEYLHSRK | |
| IMP-75 | MLSVMVSSSLVLIVFELGASEEAKPATTTIKNTKPQCRPEDYATR | 108 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLESRLEEYLHSRK | |
| IMP-76 | MLSVMVSSSLVLIVFFLGASEEAKPATTTTIKNTKPQCRPEDYAT | 109 |
| with signal peptide | RLQDLRVTFDRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-77 | MLSVMVSSSLVLIVFFLGASEEAKPAATTTIKNTKPQCRPEDYAT | 110 |
| with signal peptide | RLQDLRVTFHRVKPTLQHEDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-78 | MLSVMVSSSLVLIVFELGASEEAKPATTTTIKNTKPQCRPEDYAT | 111 |
| with signal peptide | RLQDLRVTFHRVKPTLQREDDYSVWLDGMVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-79 | MLSVMVSSSLVLIVFFLGASEEAKPATTTTIKNTKPQCRPEDYAT | 112 |
| with signal peptide | RLQDLRVTFHRVKPTLQREDDYSVWLDGTVVKGCWGCSVVDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-80 | MLSVMVSSSLVLIVFELGASEEAKPATTTTIKNTKPQCRPEDYAT | 113 |
| with signal peptide | RLQDLRITFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-81 | MLSVMVSSSLVLIVFFLGASEEAKPATTTIKNTKPQCRPEDYATR | 114 |
| with signal peptide | LQDLRVTFHRIKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-82 | MLSVMVSSSLVLIVFFLGASEEAKPATTTTIKNTKPQCRPEDYAT | 115 |
| with signal peptide | RLQDLRVTFHRVKPTLQCEDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-83 | MLSVMVESSLVLIVFFLGASEEAKPATTTTTIKNTKPQCRPEDYA | 116 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-84 | MLSVMVSSSLVLIVFFLGASEEAKPATTTTIKNTKPQCRPEDYAT | 117 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDALFSRLEEYLHSRK | |
| IMP-85 | MLSVMVSSSLVLIVFELGASEEAKPATTTTIKNTKPQCRPEDYAT | 118 |
| with signal peptide | RLQDLRVTFHRVKPTLQREDDYSVWLDGTVVKGCWGCSVMDWLLR | |
| | RYLEILFPAGDHVYPGLKTELHSMRSTLESIYKDMRQCPLLGCGD | |
| | KSVISRLSQEAERKSDNGTRKGLSELDTLFSRLEEYLHSRK | |
| IMP-86 | MLSVMVSSSLVLIVFFLGASEEAKPATTTTTIKNTKPQCRPEDYA | 119 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGMRKGLSELDTLESRLEEYLHSRK | |
| IMP-87 | MLSVMVSSSLVLIVFFLGASEEAKPATTTIKNTKPQCRPEDYATR | 120 |
| with signal peptide | LQDLRVTFHRVKPTLQREDDYSVWLDGTVVKGCWGCSVMDWLLRR | |
| | YLEIVFPAGDHVYPRLKTELHSMRSTLESIYKDMQQCPLLGCGDK | |
| | SVISRLSQEAERKSDNGTRKGLSELDTLFSRLEEYLHSRK | |
| IMP-88 | MLSVMVSSSLVLIVFFLGASEEAKPAATTTTIKNTKPQCRPEDYA | 121 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-89 | MLSVMVSSSLVLIVFFLGASEEAKPAATTTTTIKNTKPQCRPEDY | 122 |
| with signal peptide | ATRLQDLRVTFHRVKPTLQREDDYSVWLDGTVVKGCWGCSVMDWL | |
| | LRRYLEIVFPAGDHVYPGLKTELHSMRSTLESIYKDMRQCPLLGC | |
| | GDKSVISRLSQEAEKKSDNGTRKGLSELDTLFSRLEEYLHSRK | |
| IMP-90 | MLSVMVSSSLVLIVFELGASEEAKPAATTTTIKNTKPQCRPEDYA | 123 |
| with signal peptide | SRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-91 | MLSVMVSSSLVLIVFELGASEEAKPATTTTTIKNTKPQCRPEDYA | 124 |
| with signal peptide | TRLQDLRVTFHRVKPTLQREDDYSVWLDGTVVKGCWGCSVMDWLL | |
| | RRYLEIMFPAGDHVYPGLKTELHSMRSTLESIYKDMRQCPLLGCG | |
| | DKSVISRLSQEAERKSDNGTRKGLSELDTLFSRLEEYLHSRK | |
| IMP-92 | MLSVMVSSSLVLIVFFLGASEEAKSATTTIKNTKPRCRPEDYATR | 125 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLFSRLEEYLHSRK | |
| IMP-93 | MLSVMVSSSLVLIIFFLGASEEAKPATTTTIKNTKPQCRPEDYAT | 126 |
| with signal peptide | RLQDLRVTFHRVKPTLQREDDYSVWLDGTVVKGCWGCSVMDWLLR | |
| | RYLEIVFPAGDYVYPGLKTELHSMRSTLESIYKDMRQCPLLGCGD | |
| | KSVISRLSQEAERKSDNGMRKGLSELDTLFSRLEEYLHSRK | |
| IMP-94 | MLSVMVSSSLVLIVFFLGASEEAKPATTTIKNTKPQCRPEDYATR | 127 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDALFSRLEEYLHSRK | |
| IMP-95 | MLSVMVSSSLVLIVFFLGASEEAKPATTTTTIKNTKPRCRPEDYA | 128 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-96 | MLSVMVSSSLVLIVFFLGASEEAKPAATTTTTIKNTKPQCRPEDY | 129 |
| with signal peptide | ATRLQDLRVTFHRVKPTLQREDDYSVWLDGTVVKGCWGCSVMDWL | |
| | LRRYLEIVFPAGDHVYPGLKTELHSMRSTLESIYKDMRQCPLLGC | |
| | GDKSVISRLSQEAERKSDNGTRKGLSELDTLFSRLEEYLHSRK | |
| IMP-97 | MLSVMVSSSLVLIIFFLGASEEAKPATTTTTTIKNTKPQCRPEDY | 130 |
| with signal peptide | ATRLQDLRVTFHRVKPTLQREDDYSVWLDGTVVKGCWGCSVMDWL | |
| | LRRYLEIVFPAGDHVYPGLKTELHSMRSTLESIYKDMRQCPLLGC | |
| | GDKSVISRLSQEAERKSDNGTRKGLSELDTLFSRLEEYLHSRK | |
| IMP-98 | MLSVMVSSSLVLIVFFLGASEEAKPAATTTIKNTKPQCRPEDYAT | 131 |
| with signal peptide | RLQDLRVTFHRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLNELDTLFSRLEEYLHSRK | |
| IMP-99 | MLSVMVSSSLVLIVFELGASEEAKPATTTTTIKNTKPQCRPEDYA | 132 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDALFSRLEEYLHSRK | |
| IMP-100 | MLSVMVSSSLVLIVFELGASEEAKP | 133 |
| with signal peptide | ATTTTIKNTKPQCRPEDYATRLQDLCVTFHRVKPTLQREDDYSVW | |
| | LDGTVVKGCWGCSVMDWLLRRYLEIVFPAGDHVYPGLKTELHSMR | |
| | STLESIYKDMRQCPLLGCGDKSVISRLSQEAERKSDNGTRKGLSE | |
| | LDTLFSRLEEYLHSRK | |
| IMP-101 | MLSVMVSSSLVLIVFELGASEEAKPATTTTTTTIKNTKPQCRPED | 134 |
| with signal peptide | YATRLQDLRVTFHRVKPTLQREDDYSVWLDGTVVKGCWGCSVMDW | |
| | LLRRYLEIVFPAGDHVYPGLKTELHSMRSTLESIYKDMRQCPLLG | |
| | CGDKSVISRLSQKAERKSDNGTRKGLSELDTLFSRLEEYLHSRK | |
| IMP-102 | MLSVMVSSSLVLIVFELGASEEAKPAATTTTIKNTKPQCRPEDYA | 135 |
| with signal peptide | SRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKSLSELDTLESRLEEYLHSRK | |
| IMP-103 | MLSVMVSSSLVLIVFFLGASEKAKSATTTIKNTKPQCRPEDYATR | 136 |
| with signal peptide | LQDLRVTFHRVKPTLQREDDYSVWLDGTVVKGCWGCSVMDWLLRR | |
| | YLEIVFPAGDHVYSGLKTELHSMRSTLESIYKDMRQCPLLGCGDK | |
| | SVISRLSQEAERKSDNGTRKGLSELDTLFSRLEEYLHSRK | |
| IMP-104 | MLSVMVSSSLVLIVFFLGASEEAKPAATTTTTTIKNTKPQCRPED | 137 |
| with signal peptide | YATRLQDLRVTFHRVKPTLQREDDYSVWLDGTVVKGCWGCSVMDW | |
| | LLRRYLEIVFPAGDHVYPGLKTELHSMRSTLESIYKDMRQCPLLG | |
| | CGDKSVISRLSQKAERKSDNGTRKGLSELDTLFSRLEEYLHSRK | |
| IMP-105 | MLSVMVSSSLVLIVFELGASEEAKPAATTTTTMIKNTKPQCRPED | 138 |
| with signal peptide | YATRLQDLRVTFHRVKPTLQREDDYSVWLDGTVVKGCWGCSVMDW | |
| | LLRRYLEIVFPAGDHVYPGLKTELHSMRSTLESIYKDMRQCPLLG | |
| | CGDKSVISRLSQEAERKSDNGTRKGLSELDTLFSRLEEYLHSRK | |
| IMP-106 | MLSVMVSSSLVLIVFFLGASEEAKPATTTTTIKNTKPQCRPEDYA | 139 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMWQCPLLGCG | |
| | DKSVISRLSQEAERKSDNGTRKGLSELDTLFSRLEEYLHSRK | |
| IMP-107 | MLSVMVSSSLVLIVFELGASEEAKPAATTTTIKNTKPQCRPEDYA | 140 |
| with signal peptide | TRLQDFRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-108 | MLSVMVSSSLVLIVFFLGASEEAKPATIKNTKPQCRPEDYATRLQ | 141 |
| with signal peptide | DLRVTFHRVKPTLQREDDYSVWLDGTVVKGCWGCSVMDWLLRRYL | |
| | EIVFPAGDHVYPGLKTELHSMRSTLESIYKDMRQCPLLGCGDKSV | |
| | ISRLSQEAERKSDNGTRKGLSELDTLFSRLEEYLHSRK | |
| IMP-109 | MLSVMVSSSLVLIVFFLGASEEAKPAATTTTTTTIKNTKPQCRPE | 142 |
| with signal peptide | DYATRLQDLRVTFHRVKPTLQREDDYSVWLDGTVVKGCWGCSVMD | |
| | WLLRRYLEIVFPAGDHVYPGLKTELHSMRSTLESIYKDMRQCPLL | |
| | GCGDKSVISRLSQEAERKSDNGTRKGLSELDTLFSRLEEYLHSRK | |
| IMP-110 | MLSVMVSSSLVLIVFFLGASEEAKPAATTTTTTMIKNTKPQCRPE | 143 |
| with signal peptide | DYATRLQDLRVTFHRVKPTLQREDDYSVWLDGTVVKGCWGCSVMD | |
| | WLLRRYLEIVFPAGDHVYPGLKTELHSMRSTLESIYKDMRQCPLL | |
| | GCGDKSVISRLSQEAERKSDNGTRKGLSELDTLFSRLEEYLHSRK | |
| IMP-111 | MLSVMVSSSLVLIVFFLGASEEAKPATTTTTIKNTKPQCRPEDYA | 144 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLRELDTLFSRLEEYLHSRK | |
| IMP-112 | MLSVMVSSSLVLIVFFLGASEEAKPAATTTTTTTTIKNTKPQCRP | 145 |
| with signal peptide | EDYATRLQDLRVTFHRVKPTLQREDDYSVWLDGTVVKGCWGCSVM | |
| | DWLLRRYLEIVFPAGDHVYPGLKTELHSMRSTLESIYKDMRQCPL | |
| | LGCGDKSVISRLSQEAERKSDNGTRKGLSELDTLESRLEEYLHSR | |
| | K | |
| IMP-113 | MLSVMVSSSLVLIVFFLGASEEAKPAATTTTTTIKNTKPQCRPED | 146 |
| with signal peptide | YATRLQDLRVTFHRVKPTLQREDDYSVWLDGTVVKGCWRCSVMDW | |
| | LLRRYLEIVFPAGDHVYPGLKTELHSMRSTLESIYKDMRQCPLLG | |
| | CGDKSVISRLSQEAERKSDNGTRKGLSELDTLFSRLEEYLHSRK | |
| IMP-114 | MLSVMVSSSLVMIVFFLGASEEAKPATTTTTIKNTKPQCRPEDYA | 147 |
| with signal peptide | TRLQDLCVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLESRLEEYLHSRK | |
| IMP-115 | MLSVMVSSSLVLIVFELGASEEAKPAAATTTTTTTTIKNTKPQCR | 148 |
| with signal peptide | PEDYATRLQDLRVTFHRVKPTLQREDDYSVWLDGTVVKGCWGCSV | |
| | MDWLLRRYLEIVFPAGDHVYPGLKTELHSMRSTLESIYKDMRQCP | |
| | LLGCGDKSVISRLSQEAERKSDNGTRKGLSELDTLFSRLEEYLHS | |
| | RK | |
| IMP-116 | MLSVMVSSSLVLIVFFLGASEEAKPAATTTTTTTIKNTKPQCRPE | 149 |
| with signal peptide | DYATRLQDLRVTFHRVKPTLQREDDYSVWLDGTVVKGCWGCSVMD | |
| | WLLRRYLEIVFPAGDHVYPGLKTELHSMRSTLESIYKDMRQCPLL | |
| | GCGDKSVISRLSQEAERKSDNGTRKGLSELDTLFIRLEEYLHSRK | |
| IMP-117 | MLSVMVSSSLVLIVFELGASEEAKPATTTTTIKNTKPQCRPEDYA | 150 |
| with signal peptide | TRLQDLRVTFHRVKPTLQREDDYSVWLDGTVVKGCWGRSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-118 | MLSVMVSSSLVLIVFELGASEEAKPATTTTTIKNTKPQCRPEDYA | 151 |
| with signal peptide | TRLQDLRVTFHRVKPTLQREDDYSVWLDGTVVKGCWGCSVMDWLL | |
| | RRYLEIVFPAGDHVYPGLKTELHSMRSTLESICKDMRQRPLLGCG | |
| | DKSVISRLSQEAERKSDNGTRKGLSELDTLFSRLEEYLHSRK | |
| IMP-119 | MLSVMVSSSLVLIVFFLGASEEAKPAATTTTTTTTTIKNTKPQCR | 152 |
| with signal peptide | PEDYATRLQDFRVTFHRVKPTLQREDDYSVWLDGTVVKGCWGCSV | |
| | MDWLLRRYLEIVFPAGDHVYPGLKTELHSMRSTLESIYKDMRQCP | |
| | LLGCGDKSVISRLSQEAERKSDNGTRKGLSELDTLFSRLEEYLHS | |
| | RK | |
| IMP-120 | MLSVMVSSSLVLIVFFLGASEEAKPATIKNTKPQCRPEDYATRLQ | 153 |
| with signal peptide | DLRVTFHRVKPTLPGHQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAERKSDNGTRKGLSELDTLFSRLEEYLHSRK | |
| IMP-121 | MKTNTKIILFCYVILSLYVESCAIASAKKCDDVSEDYILKDLRSE | 154 |
| with signal peptide | FSKIKSFVQDNDQENMMLLSQSMLDKLTSRIGCKSLSDMIKFYLN | |
| | DVLPNAEKIEHMKNKITSIGEKLKSLKEKLISCDELHCENHDEIK | |
| | TVKTIFNKLKDKGIYKAMGEFDIFINYLEKYIVKK | |
| IMP-122 | MKTNTKIILFCYVILSLYVFSCAIASAKKCNDVSFDYILKDLRSE | 155 |
| with signal peptide | FSKIKSFVQDNDQENMMLLSQSMLDKLTSRIGCKSLSDMIKFYLN | |
| | DVLPNAEKIEHMKNKITSIGEKLKSLKEKLISCDFLHCENHDEIK | |
| | TVKTIFNKLKDKGIYKAMGEFDIFINYLEKYIVKK | |
| IMP-123 | MKTNTKIILFCYVIFLSLYVESCVVASAKKCDDVSFDYILKDLRS | 156 |
| with signal peptide | EFSKIKSFVQDNDQENMMLLSQSMLDKLTSRIGCKSLSDMIKFYL | |
| | NDVLPNAEKIEHMKNKITSIGEKLKSLKEKLISCDFLHCENHDEI | |
| | KTVKTIFNKLKDKGIYKAMGEFDIFINYLEKYIVKK | |
| IMP-124 | MKTNTKIILFCYVILSLYVESCVVAYAKKCDDVSFDYILKDLRSE | 157 |
| with signal peptide | FSKIKSFVQNNDQENMMLLSQSMLNKLTSCIGCKSLSDMIKFYLN | |
| | DVLPNAEKIEQIKNIITSIGEKLKSLKEKLISCDFLHCENNDEIK | |
| | TVKAIFNKLKDKGIYKAMGEFDIFINYVEKYIVKT | |
| IMP-125 | MKTSTKIILFCYVILSLYVFSCVVASAKKCDDVSFDYILKDLRSE | 158 |
| with signal peptide | FIKIKSFVQNNDQENMMLLSQSMLDKLTSCIGCKSLSDMIKFYLN | |
| | DVLPNAEKIEQIKNIITSIGEKLKSLKEKLISCDFLHCENNDEIK | |
| | TVKAIFNKLKDKGIYKAMGEFDIFINYVEKYIVKT | |
| IMP-126 | MKTNTKIILFCYVILYVESCTVASAKKCDDVSFDYILKDLRSEFS | 159 |
| with signal peptide | KIKSFVQNNDKENMMLLSQSMLDKLTSCIGCKSLSDMIKFYLNDV | |
| | LPNAEKIEHIKNKITSIGEKLKSLKEKLISCDFLHCENHDEIKAV | |
| | KTIFNKLKDKGIYKAMGEFDIFINHLEKYIVKK | |
| IMP-127 | MKTSTKIILFCYVILSLYVESCVVASAKKCDDVSFDYILKDLRSE | 160 |
| with signal peptide | FIKIKSFVQNNDQENMMLLSQSMLDKLTSRIGCKSLSDMIKFYLN | |
| | DVLPNAEKIEQIKNIITSIGEKLKSLKEKLISCDFLHCENNDEIK | |
| | TVKAIFNKLKDKGIYKAMGEFDIFINYVEKYIVKT | |
| IMP-128 | MKTNTKIILFCYVILYLESCTVASAKKCDDVSFDYILKDLRSEFS | 161 |
| with signal peptide | KIKSFVQNNDKENMMLLSQSMLDKLTSCIGCKSLSDMIKFYLNDV | |
| | LPNAEKIEHIKNKITSIGEKLKSLKEKLISCDFLHCENHDEIKAV | |
| | KTIFNKLKDKGIYKAMGEFDIFINHLEKYIVKK | |
| IMP-129 | MLSVMVSSSLVLIVFFLGASEEAKPATTTTTIKNTKPQCRPEDYA | 162 |
| with signal peptide | TRLQDLRVTFHRVKPTLDDYSVWLDGTVVKGCWGCSVMDWLLRRY | |
| | LEIVFPAGDHVYPGLKTELHSMRSTLESIYKDMRQCPLLGCGDKA | |
| | VISRLSQEAERKSDNGTRKGLSELDTLFSRLEEYLHSRK | |
| IMP-130 | MKTNTKIILFCYVILYLESCTVASAKKCDDVSFDYILKDLRSEFS | 163 |
| with signal peptide | KIKSFVQNNDKENMMLLSQSMLDKLTRCIGCKSLSDMIKFYLNDV | |
| | LPNAEKIEHIKNKITSIGEKLKSLKERLISCDFLHCENHDEIKAV | |
| | KTIFNELKDKGIYKAMGEFDIFINHLEKYIVKK | |
| IMP-131 | MERRLVVTLQCLVLLYLAPECGGTDQCDNFPQMLRDLRDAFSRVK | 164 |
| with signal peptide | TFFQTKDEVDNLLLKESLLEDFKGYLGCQALSEMIQFYLEEVMPQ | |
| | AENQDPEAKDHVNSLGENLKTLRLRLRRCHRFLPCENKSKAVEQI | |
| | KMPLTSCRKKEFTKP | |
| IMP-132 | MGSRRLSRCSLATAVCLVAIVVAVAAKGRDSKPSPACDPMHGALA | 165 |
| with signal peptide | GIFKELRTTYRSVRETLQTKDTVYYVSLFHEQLLQEMLSPVGCRV | |
| | TNELMQHYLDGVLPRAFHCGYDNTTLNALHELSSSLSTLYQHMLK | |
| | CPALACTGQTPAWTQFLDTEHKLDPWKGTVKATAEMDLLLNYLET | |
| | FLLQS | |
| IMP-133 | MLSVMVSSSLVLIVFLLGASEEAKPATTTIKNTKPQCRPEDYATR | 166 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCEAERKSDN | |
| | GTRKGLSELDTLESRLEEYLHSRK | |
| IMP-134 | MRRRRSFGVVVVGAIGTLLMMAVVVLSAHDHEHKVPPACDPVHGN | 167 |
| with signal peptide | LAGIFKELRTIYTSIREGLQKKDTVYYTSLENDRVLQEMLSPMGC | |
| | RVTNEIMEHYLDGVLPRASHLDYDNSTLNGLHAFASSMQALYQHM | |
| | LKCPALACTGKTPAWMYFLEVEHKLNPWRGTAKAAAEADLLLNYL | |
| | ETFLLQF | |
| IMP-135 | MGSRPARMCGLSNLLCLLLVVLVAVVIHRGCGASKPPVDCDPIHG | 168 |
| with signal peptide | TLSRIIKEVRTGYGSIKQALQSKDTVYYVSLFHENLLNEMLSPVG | |
| | CRVTNELMQHYLDGVLPRAFQCGYDNTTLDGLHSLVSSLDALYKH | |
| | MLKCPALACTGQTPAWTQFLETEHKLDPWKGTIKATAEMDLLVNY | |
| | LETFLAQS | |
| IMP-136 | MLSMMVSSSLVLIVFFLGASEEAKPATTTTTIKNTKPQCRPEDYA | 169 |
| with signal peptide | TRLQDLRVTFHRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISR | |
| IMP-137 | MLSVMVSSSLVLIVFELGASEEAKPATTTIKNTKPQCRPEDYATR | 170 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCVSVSVAAL | |
| | SAQR | |
| IMP-138 | MININILSLLILILSIYANAIIDTCYDDQERERTKSNSISSVTPE | 171 |
| with signal peptide | MCKGLKQLVSTKLKDARQKEKSVRDYFTSRDNDLDEMLLQGVKET | |
| | HKKTCGCYVLYLLLSFYGKTIRDTIQSNKHKNLNTELTNLAVSVL | |
| | SLEDLLEACGITCNPKKDSLLKRIEEYMKEHGDDAIYKVIGEIEF | |
| | LFQAIEKHVY | |
| IMP-139 | MINISINILSLLILILSIYANSIIDMCYDDQERERTKSNSISSIT | 172 |
| with signal peptide | PDMCKGLKQLVATKLKDARQKEKLVNSYFTSRDNDLTYMLLQGVR | |
| | ETHKKPCGCYVLYLLLTFYRKTIKDTIQSKKHESINTELTNLAVT | |
| | VLSLEDLLEACGITCNPKKDSLLKRIEGYTKEHGDDAIYKVIGEI | |
| | DELFQAIERHVY | |
| IMP-140 | MININILSLLILILSIYANAIIDTCYDDQERERTKSNSISSVTPE | 173 |
| with signal peptide | MCKGLKQLVATKLKDARQKEKLVNDYFTGRDNDLSYMLLQGVRET | |
| | HKKPCGCYVLYLLLSFYRKTIRDTIQSNKHASINAELTNLAVSVL | |
| | SLEDLLDACGITCNPKKDSLLKRIEEYMKEHGDDAIYKLIGEIEF | |
| | LFQAIERHVYT | |
| IMP-141 | MININILSLLILILSIYANAIIDTYDEDEDEDSIKLSSIGSITPE | 174 |
| with signal peptide | MCKNLKQLVASKLKDIRQKEKSLRDYFTNLDDELDYMLLQGVGEN | |
| | HKKKCGCYILHLLLKFYSKTIRNTIQSEKHKNVNLELTNIAVSML | |
| | ALEDLLEKCGITCNPKKDPLLKRIEDYMKQHGDDGVNKAIAELEF | |
| | LFQMIEKQVYI | |
| IMP-142 | MARFIYVVLLCLVEDAAQSAAQCRKGTITSRLKMLRTAFEKVREF | 175 |
| with signal peptide | YEDRDEEETALASTEHLHGPESCSVIDELITHYTKCVIPAANEEE | |
| | GADLLSLDTLQVALENVKGLLANCQEEFGCKPPFSMRDYKKQYRQ | |
| | LNKEKNAGMIKAMGELGMLENGIEERVIGM | |
| IMP-143 | MMLALAMMLMALGPLSTNAMYVQHGSDYCTTTVHADIASAISGMR | 176 |
| with signal peptide | AEYDSGLGHYFKSLVPHPDNPYDTDDYKYMINNTNSYNCHALQST | |
| | INALLGMYGYVDIDEPHQLAMMKLATHTMQAAMLINKCAKQLGCY | |
| | HIPFDVETLHEAHPDDVMASLDTALNLMSMVTNEI | |
| IMP-144 | MARFIYVVLLCLVEDAAQSAAQCRKGTITSRLKMLRTAFEKVREF | 177 |
| with signal peptide | YEDSDEEETALASTEHLHGPESCSVIDELITHYTKCVIPAANEEE | |
| | GADLLSLDTLQVALENVKGLLANCQEEFGCKPPFSMRDYKKQYRQ | |
| | LNKEKNAGMIKAMGELGMLENGIEERVIGM | |
| IMP-145 | MFQLEGIVLLVYLANWVSPATIKCVGMSTLENPELIQLRRLFGDG | 178 |
| with signal peptide | IKDFFQNKDEDLDNAFLNEDVQRELASDCGCDHLMDMLSLYVNDT | |
| | IPKGMKTEDAPSGLGQMGQLMSSLYRKMDMCWSELGCSHNTRLTL | |
| | QEYADKKGGWDNKALGESDILFDALELFFSKIK | |
| IMP-146 | MMTTLALVMTLMALSTNAMYVQHGSDYCTTTVRADIASAISGMRA | 179 |
| with signal peptide | EYNNGLGDYFKSLAPHPNNPYDTDDYKYMINSTNSYNCHALQSTI | |
| | NALLGMYGYVDIDEPHQLAMMKLATHTMQTAMLLNKCAAQLGCYH | |
| | IPFDVETLHEAHPNDVMASLDTALNLMSMVTNEI | |
| IMP-147 | MITLALVMTLMALGPLSTNAMYARRSGDYCTTTVRADIASAISGM | 180 |
| with signal peptide | RAEYNSGLRDYFKSLVPHPDNPYDTDDYKYMLNNTNSYNCHALQS | |
| | TINALLGMYGYVDIDESHQLAMMKLATHTMQTAMMLNKCAAQLGC | |
| | YHIPFDLETLHEAHPDDVMASLDTALNLMSMITNEI | |
| IMP-148 | MMTTLALVMTLMALATNAMYVQHGSDYCTTTVRADIASAISGMRA | 181 |
| with signal peptide | EYNNGLGDYFKSLAPHPNNPYDTDDYKYMINSTNSYNCHALQSTI | |
| | NALLGMYGYVDIDEPHQLAMMKLATHTMQTAMLLNKCAAQLGCYH | |
| | IPFDVETLHEAHPDDVMASLDTALNLMSMVTNEI | |
| IMP-149 | MSGTSNKKFVELVAIAVAICMMSSVSSNVHSGTEDNPCTNSKTVL | 182 |
| with signal peptide | NTLLNQIKQEYINNLLPYYKALTPKPVDVEDDSYTYSIQSTDYNC | |
| | YTIYETLNFLLGDVFPRATTDATVRLSLAKIATSSQQASMLMNLC | |
| | KKELACGPAPFDMIKLYHDTKEYGADNIMGTLDTPFQYFVIV | |
| IMP-150 | MLALAMVLMALGPLSTNAMYVQHGRGDYCTTTVRADIASAISGMR | 183 |
| with signal peptide | AEYDSGLGHYFKSLVPHPDNPYDTDDYKYMINNTNSYNCHALQST | |
| | INALLGMYGYVDIDEPHQLAMMKLATHTMQTAMLINKCAEQLGCY | |
| | HIPFDVEILHEAHPDDVMASLDTALNLMSMVTNEI | |
| IMP-151 | MFLAVLLTATIFFEARGAPATTPKDSCVYLIGQTPQLLRQLRNAY | 184 |
| with signal peptide | QAIIGADGSGVDEDDMPIYPSDVMNELASTSVACDAIKKVLTMNI | |
| | GILPNVTAAYPDKKSEVDEIGDNLSRLHQNIVNCRDELKCEDLPH | |
| | WHQMAENYKEKPMQGFSEMDFVFQSVEKELVAKDVKNMKTKRKH | |
| IMP-152 | MLKQIIVVCIVAMAAVFADDDPCTNVKTQLNTLENQIKTEYDTNL | 185 |
| with signal peptide | KTYYQSIAPSAFDPENNTNYLYSVQGNDYKCYTIFETLSFLMGDV | |
| | YPRATTNESVRLSLAKVATSSTQGAMVMNLCRQQLGCGPPPFDAK | |
| | TLYDDRAEYGADDIMATLDTALAKFKLVLESENVV | |
| IMP-153 | MLKQIIVVCIVAMAAVFADDDPCTNVKTQLNTLENQIKTEYDTNL | 186 |
| with signal peptide | KTYYQSIAPSAFDPENNTNYLYSVQGNDYKCYTIFETLSFLMGDV | |
| | YPRATTNESVRLSLAKVATSSTQGAMVMNLCREQLGCGPPPFDAK | |
| | TLYDDRAEYGADDIMATLDTALAKFKLVLESENVV | |
| IMP-154 | MITLALVMTLMALGPLSTNGVHARRRGDYCTTTVRADIASAISGM | 187 |
| with signal peptide | RAEYNSGLGDYFKSLVPHPDNPYDTDDYKHMIDNANSYNCHALQS | |
| | TINALLGMYGYVDIDEPHQLAMMKLATHTMQTAMLLNKCAAQLGC | |
| | YHIPFDLETLREAPPADVMASLDTALNLMSMITNEI | |
| IMP-155 | MDAQFLLLIVLALPASFAASLSTHYNNYDLTRIATIDKDVCKRVA | 188 |
| with signal peptide | QHINDDFVNMRKLYETQLKNYFQQLVPNPTDVFKDDSYMYMINGT | |
| | DYNCHIIYETMRFLSGDVFPFATETEAELQYMWKMMLGVSQLSAY | |
| | IGNCYQYFKCGPAPFDPQVLYHDRELFHADTVMAYLDTAFSHETL | |
| IMP-156 | MKLYFYCIFFYKIIVTISLNCGIEHNELNNIKNIFFKVRNVVQAD | 189 |
| with signal peptide | DVDHNLRILTPALLNNITVSETCFFIYDMFELYLNDVFVKYTNTA | |
| | LKLNILKSLSSVANNFLAIFNKVKKRRVKKNTVNVLEIKKLLLID | |
| | NNCKKLFSEIDIFLTWVMAKI | |
| IMP-157 | MKLYFYCIFFYKIIVTISLNCGIEHNELNNIKNIFFKVRNVVQAD | 190 |
| with signal peptide | DVDHNLRILTPALLNNITVSETCFFIYDMFELYLNDVFVKYTNTA | |
| | LKLNILKSLSSVANNFLAIFNKVKKRRVKKNNVNVLEIKKLLLID | |
| | NNCKKLFSEIDIFLTWVMAKI | |
| IMP-158 | MFLAVLLTATIFFEARGAPATTPKDSCVYLIGQTPQLLRQLRNAY | 191 |
| with signal peptide | QAIIGADGSGVDEDDMPIYPSDVMNELASTSVACDAIKKVLTMNI | |
| | GILPNVTAAYPDKKSEVDEIGDNLSRLHQNIVNCVSRTQHLCYD | |
| IMP-159 | MFRASLLCCLVLLAGVWADNKYDSESGDDCPTLPTSLPHMLHELR | 192 |
| with signal peptide | AAFSRVKTFFQMKDQLDNMLLDGSLLEDFKGYLGCQALSEMIQFY | |
| | LEEVMPQAENHSPDQDKNKVNSLGEKLKTLRVRLRRCHRFLPCEN | |
| | KSKAVEQVKSAFSKLQEKGVYKAMSEFDIFINYIEAYMTTKMKN | |
| IMP-160 | MFRASLLCCLVLLAGVWADNKYDSESGNDCPTLPTSLPHMLHELR | 193 |
| with signal peptide | AAFSRVKTFFQMKDQLDNMLLDGSLLEDFKGYLGCQALSEMIQFY | |
| | LEEVMPQAENHSTGQEKDKVNSLGEKLKTLRVRLRRCHRFLPCEN | |
| | KSKAVEQVKSAFSKLQEKGVYKAMSEFDIFINYIEAYMTTKMKN | |
| IMP-161 | MFRASLLCCLVLLAGVWADNKYDSESGNDCPTLPTSLPHMLHELR | 194 |
| with signal peptide | AAFSRVKTFFQMKDQLDNMLLDGSLLEDFKGYLGCQALSEMIQFY | |
| | LEEVMPQAENHSTDQEKDKVNSLGEKLKTLRVRLRRCHRFLPCEN | |
| | KSKAVEQVKSAFSKLQEKGVYKAMSEFDIFINYIEAYMTTKMKN | |
| IMP-162 | MFRASLLCCLVLLAGVWADNKYDSESGDDCPTLPTSLPHMLHELR | 195 |
| with signal peptide | AAFSRVKTFFQMKDQLDNMLLDGSLLEDFKGYLGCQALSEMIQFY | |
| | LEEVMPQAENHSTGQEKDKVNSLGEKLKTLRVRLRRCHRFLPCEN | |
| | KSKAVEQVKSAFSKLQEKGVYKAMSEFDIFINYIEAYMTTKMKN | |
| IMP-163 | MFRALLLCCLALLAGVWADNRYDGQDGNDCPTLPTSLPHMLHELR | 196 |
| with signal peptide | AAFSRVKTFFQMKDQLDNMLLDGSLLEDFKGYLGCQALSEMIQFY | |
| | LEEVMPQAENHSPDQDKNKVNSLGEKLKTLRVRLRRCHRELPCEN | |
| | KSKAVEQVKSAFSKLQEKGVYKAMSEFDIFINYIEAYMTTKMKN | |
| IMP-164 | MLSVMVSSSLVLIVFFLGASEEAKPATTAIKNTKPQCRPEDYATR | 197 |
| with signal peptide | LQDLRVTFHRVKPTLQREDDYSVWLDGTVVKGCWGCSVMDWLLRR | |
| | YLEIVFPAGDHVYPGLKTELHSMRSTLESIYKDMRQCPLLGCGDK | |
| | SVISRLSQEAERKSDNGTRKGLSELDTLESRLEEYLHSRK | |
| IMP-165 | MLSVMVSSSLVLIVFFLGAFEEAKPATTTTIKNTKPQCRPEDYAT | 198 |
| with signal peptide | RLQDLRVTFDRVKPTLQREDDYSVWLDGTVVKGCWGCSVMDWLLR | |
| | RYLEIVFPAGDHVYPGLKTELHSMRSTLESIYKDMRQCPLLGCGD | |
| | KSVISRLSQEAEKKSDNGTRKGLSELDTLFSRLEEYLHSRK | |
| IMP-166 | MLSVMVSSSLVLIVFFLGASEEAKPATTTTTIKNTKPQCRPEDYA | 199 |
| with signal peptide | TRLQDLRVTFYRVKPTLQREDDYSVWLDGTVVKGCWGCSVMDWLL | |
| | RRYLEIVFPAGDHVYPGLKTELHSMRSTLESIYKDMRQCPLLGCG | |
| | DKSVISRLSQEAERKSDNGTRKGLSELDTLFSRLEEYLHSRK | |
| IMP-167 | MLSVMVSSSLVLIVFFLGASEEAKPATTTTTIKNTKPQCRPEDYA | 200 |
| with signal peptide | TRLQDLRVTFHRVKPTLDDYSVWLDGTVVKGCWGCSVMDWLLRRY | |
| | LEIVFPAGDHVYPGLKTELHSMRSTLESIYKDMRQCPLLGCGDKS | |
| | VISRLSQEAERKSDNGTRKGLSELDTLFSRLEEYLHSRK | |
| IMP-168 | MKTNTKIILFCYVIFLSLYVFSCVVASTKKCDDVSFDYILKDLRS | 201 |
| with signal peptide | EFSKIKSFVQDNDQENMMLLSQSMLDKLTSRIGCKSLSDMIKFYL | |
| | NDVLPNAEKIEHMKNKITSIGEKLKSLKEKLISCDFLHCENHDEI | |
| | KTVKTIFNKLKDKGIYKAMGEFDIFINYLEKYIVKK | |
| IMP-169 | MLSVMVSSSLVLIVFELGASEEAKPATTTIKNTKPQCRPEDYATR | 202 |
| with signal peptide | LQDLRVTFHRVKPTLQREDDYSVWLDGDHVYPGLKTELHSMRSTL | |
| | ESIYKDMRQCPLLGCGDKSVISRLSQEAERKSDNGTRKGLSELDT | |
| | LESRLEEYLHSRK | |
| IMP-170 | MSNFRILSLLIFSLIVHVNAMIGTCYDEDEEIERLKSNSISSITP | 203 |
| with signal peptide | GMCRNLKHSVMIRLIDARQIEASIRSYFTDGDNNLSEMLLQGIRE | |
| | ISKKKCGCYILNLMLRFYIQTIKHTILSNKHKDMNLELTNLAVTI | |
| | LSLESLLEKCGVTCNPVKDPLLTRIEEYTRKHGDNAIYKTIGELE | |
| | FLFDAIEKFV | |
| IMP-171 | MARFIYVVLLCLVEDAAQSAAQCRKGTITIRLKMLRTAFEKVREF | 204 |
| with signal peptide | YEDRDEEETALASTEHLHGPESCSVIDELITHYTKCVIPAANEEE | |
| | GADLRSLDTLQFALENVKGLLANCQEEFGCKPPFSMRDYKKQYRQ | |
| | LNKEKNAGMIKAMGELGMLENGIEERVIGM | |
| IMP-172 | MARFIYVVLLCLVEDAAQSAAQCRKGTITIRLKMLRTAFEKVREF | 205 |
| with signal peptide | YEDRDEEETALASTEHLHGPESCSVIDELITHYTKCVIPAANEEE | |
| | GADLRSLDTLQFALENVKGLLANCQEEFGCKPPFSMRDYKKQYRQ | |
| | LNKEKNAGMIKAMGELGMLENGIEERVNGM | |
| IMP-173 | MARFIYVVLLCLVEDAAQSAAQCRKGTITIRLKMLRTAFEKVREF | 206 |
| with signal peptide | YEDRDEEETALASTEHLHGPESCSVIDELITHYTKCVIPAANEEE | |
| | GADLLSLDTLQFALENVKGLLANCQEEFGCKPPFSMRDYKKQYRQ | |
| | LNKEKNAGMIKAMGELGMLENGIEERVIGM | |
| IMP-174 | MARFIYVVLLCLVEDAAQSAAQCRKGTITSRLKMLRTAFEKVREF | 207 |
| with signal peptide | YEDRDEEETALASTEHLHGPESCSVIDELITHHTKCVIPAANEEE | |
| | GADLLSLDTLQVALENVKGLLANCQEEFGCKPPFSMRDYKKQYRQ | |
| | LNKEKNAGMIKAMGELGMLENGIEERVIGM | |
| IMP-175 | MLSVMVSSSLVLIVFLLGASEEAKPATTTIKNTKPQCRPEDYATR | |
| with signal peptide | LQDLRVTFHRVKPTLVGHVGDHVYPGLKTELHSMRSTLESIYKDM | |
| | RQCEAERKSDNGTRKGLSELDTLFSRLEEYLHSRK | |
| IMP-273 | MHSSALLCCLVLLTGVRASPGQGTQSENSCTHEPGNLPNMLRDLR | 209 |
| with signal peptide | DAFSRVKTFFQMKDQLDNLLLKESLLEDFKGYLGCQALSEMIQFY | |
| | LEEVMPQAENQDPDIKAHVNSLGENLKTLRLRLRRCHRFLPCENK | |
| | SKAVEQVKNAFNKLQEKGIYKAMSEFDIFINYIEAYMTMKIRN | |
| IMP-274 | MHSSALLCCLVLLTGVRASPGQGTQSENSCTHFPGYLPNMLRDLR | 210 |
| with signal peptide | DAFSRVKTFFQMKDQLDNLLLKESLLEDFKGYLGCQALSEMIQFY | |
| | LEEVMPQAENQDPDIKAHVQSLGENLKDLRLWLRRCHRFLPCENK | |
| | SKAVEQVKNAFNKLQEKGIYKAMSEFDIFINYIEAYMTMKIRN | |
| IMP-275 | MHSSALLCCLVLLTGVRASPGQGTQSENSCTHEPGNLPNMLRALR | 211 |
| with signal peptide | DAFSRVKTFFQMKDQLDNLLLKESLLEDFKGYLGCQALSEMIQFY | |
| | LEEVMPQAENQDPDIKAHVNSLGENLKTLRLRLRRCHRFLPCENK | |
| | SKAVEQVKNAFNKLQEKGIYKAMSEFDIFINYIEAYMTMKIRN | |
| IMP-1 | RHCMFGDSLRNSPDMKNMLQDLRGGYSGSGIKRTFQGKDTLDSML | 212 |
| without signal | LTQSLLDDFKGYLGCQALSEMIQFYLEEVMPQAENHGPTDSVKQL | |
| peptide | GEKLHTLNQKFGECPRWFPCYYNTTPAVENVKSVESKLQERGVYK | |
| | AMSEFDIFINYIETYTTMK | |
| IMP-2 | CQLESGEALPLGSRSADSRSVDGQRVPAPQNNYPGLLRDLRLGYE | 213 |
| without signal | GFKQKVTDSHPDETLLGSSRLAGDLKGPLRCQALSEMIQFLLQVV | |
| peptide | LPDAENSRQDLRSQFSTLGDRITGLRQQLRRDPTVFPCESRSDGV | |
| | SDLRSAYTRLGSTGAEKVLSEFDIFINYIEAYVTSV | |
| IMP-3 | YCVEYAESDEDRQQCSSSSNFPASLPHMLRELRAAFGKVKTFFQM | 214 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-4 | YCTSCSHHQCTEDENQKQDCEDANHSLPHMLRELRAAFGKVKTFF | 215 |
| without signal | QMKDQLHSLLLTQSLLDDFKGYLGCQALSEMIQFYLEEVMPQAEN | |
| peptide | HGPEEHDNSLSEHGPDVKEHVNSLGEKLKTLRLRLRRCHRELPCE | |
| | NKSKAVEKVKRVESELQERGVYKAMSEFDIFINYIETYMTT | |
| IMP-5 | RSPKNKPSIDCNPQTGDFVNMLKSMRQDYSRIRDTLHDRDKLHSS | 216 |
| without signal | LLTGALLDEMMGYSGCRTTLLLMEHYLDTWYPAAYRHHLYDNQTL | |
| peptide | VVVDRMGSTLVALLKAMVQCPMLACGAPSPAMDKMLQQEAKMKKY | |
| | TGVYKGISETDLLLGYLELYMMKEKR | |
| IMP-6 | HEHKVPPACDPVHGNLAGIFKELRAIYASIREALQKKDTVYYTSL | 217 |
| without signal | FNDRVLQEMLSPMGCRVTNELMEHYLDGVLPRAAHFDYDNSTLNG | |
| peptide | LHAFTSSMQALYQHMLKCPALACTGKTPAWMYFLEVEHKLNPWRG | |
| | TAKAAAEADLLLNYLETFLLQF | |
| IMP-7 | KGRDSKPSPACDPMHGALAGIFKELRTTYRSVREALQTKDTVYYV | 218 |
| without signal | SLFHEQLLQEMLSPVGCRVTNELMQHYLDGVLPRAFHCGYDNATL | |
| peptide | NALHALSSSLSTLYQHMLKCPALACTGQTPAWTQFLDTEHKLDPW | |
| | KGTVKATAEMDLLLNYLETELLQS | |
| IMP-8 | ATTTIKNTKPQCRPEDYATRLQDLRVTFHRVKPTLQREDDYSVWL | 219 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-12 | YCVEYEESEEDRQQCSSSNFPASLPHMLRELRAAFGKVKTFFQMK | 220 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKP | |
| IMP-13 | YCVEYEESEEDRQQCSSSNFPASLPHMLRELRAAFGKVKTFFQMK | 221 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKT | |
| IMP-14 | YCVEYEESEEDRQQCSSSNFPASLPHMLRELRAAFGKVKTFFQMK | 222 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKS | |
| IMP-15 | YCVEYKESEEDRQQCSSSSFPASLPHMLRELRAAFGKVKTFFQMK | 223 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTK | |
| IMP-16 | YCVEYAESDEDKQQCSGSNEPASLPHMLRELRAAFGKVKTFFQMK | 224 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKM | |
| IMP-17 | YCVEYEESEEDKQQCGSNGGPASLPHMLRELRAAFGKVKTFFQMK | 225 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKM | |
| IMP-18 | YCVEYAESDEDRQQCSGSNFPASLPHMLRELRAAFGKVKTFFQMK | 226 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKM | |
| IMP-19 | YCVEYEESEEDKQQCSSSSNFPASLPHMLRELRAAFGKVKTEFQM | 227 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-20 | YCIQYEESEEDKQQCSSSNFPASLPHMLRELRAAFGKVKTFFQMK | 228 |
| without signal | DQLNSMLLTQSLLDDFKGYLGCQALSEMIRFYLEEVMPQAENHGP | |
| peptide | DIKEHVNSLGEKLKTLRLRLRRCHRFLPCENKSKAVEQVKRVENM | |
| | LQERGVYKAMSEFDIFINYIESYMTTKM | |
| IMP-21 | YCVEYEESEEDRQQCSSSNFPASLPHMLRELRAAFGKVKTFFQMK | 229 |
| without signal | DQLNSMLLTQSLLDDFKGYLGCQALSEMIQFYLEEVMPQAENHGP | |
| peptide | DIKEHVNSLREKLKTLRLRLRRCHRFLPCENKSKAVEQVKRVENM | |
| | LQERGVYKAMSEFDIFINYIESYMTTKT | |
| IMP-22 | YCVEYEESEEDKQQCGSSSNFPASLPHMLRELRAAFGKVKTEFQM | 230 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-23 | YCVEYAESDEDKQQCSGSNFPASLPHMLRELRAAFGKVKTFFQMK | 231 |
| without signal | DQLNSMLLTQSLLDDFKGYLGCQALSEMIQFYLEEVMPRAENHGP | |
| peptide | DIKEHVNSLGEKLKTLRLRLRRCHRFLPCENKSKAVEQVKRVENM | |
| | LQERGVYKAMSEFDIFINYIESYMTTKM | |
| IMP-24 | YCVEYEESEEDRQQCSGSSNFPASLPHMLRELRAAFGKVKTFFQM | 232 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-25 | YCVEYEESDEDRQQCSSSSNEPASLPHMLRELRAAFGKVKTFFQM | 233 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-26 | YCVEYEESDEDKQQCSSSTGAPASLPHMLRELRAAFGKVKTEFQM | 234 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-27 | YCVEYEETKEDEQQCSSSSNFPASLPHMLRELRAAFGKVKTFFQM | 235 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-28 | YCVEYEESEEDRQQCSSSNFPASLPHMLRELRAAFGKVKTFFQMK | 236 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGDYKAMSEFDIFINYIESYMTTKS | |
| IMP-29 | YCVEYEESEEDRQQCSSSSNFPASLPHMLRELRAAFGKVKTFFQM | 237 |
| without signal | KDQLNSMLLTQSLLDDFKGYLGCQALSEMIQFYLEEVMPQAENHG | |
| peptide | PDIKEHVNSLGEKLKTLRLRLRRCHRELPCEDKSKAVEQVKRVEN | |
| | MLQERGVYKAMSEFDILINYIESYMTTKM | |
| IMP-30 | YCVEYEESDEDRQQCSSSSNEPASLPHMLRELRAAFGKVKTEFQM | 238 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMATKM | |
| IMP-31 | DNRYDGQDGNDCPTLPTSLPHMLHELRAAFSRVKTFFQMKDQLDN | 239 |
| without signal | MLLDGSLLEDFKGYLGCQALSEMIQFYLEEVMPQAENHSTDQEKD | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-32 | YCVEYLESREDEQQCSSSSNFPASLPHMLRELRAAFGKVKTFFQM | 240 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-33 | YCVEYEESKEDEQQCSGSNGASASLPHMLRELRAAFGKVKTFFQM | 241 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-34 | YCVEYLESGEDEQQCGSSSNFPASLPHMLRELRAAFGKVKTFFQM | 242 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-35 | YCVEYLESREDEQQCSGSNGASASLPHMLRELRAAFGKVKTFFQM | 243 |
| without signal | KDQLNSMLLTQSLLDDFKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIESYMTTKM | |
| IMP-36 | ESENNCTHFPTSLPHMLHELRAAFSRVKTFFQMKDQLDNMLINGS | 244 |
| without signal | LLEDFKGYLGCQALSEMIQFYLEEVMPQAENHSGGGGPDIKEHVN | |
| peptide | SLGEKLKTLRVRLRRCHRFLPCENKSKAVEQVKSAFSKLQEKGVY | |
| | KAMSEFDIFINYIEAYMTTKMKNKK | |
| IMP-37 | YCVEYLESDEDKQHCSSSNGASASSPHMLRELRAAFGKVKTFFQM | 245 |
| without signal | KDQLNSMLLTQSLLDDEKGYLGCQALSEMIQFYLEEVMPQAENHG | |
| peptide | PDIKEHVNSLGEKLKTLRLRLRRCHRELPCENKSKAVEQVKRVEN | |
| | MLQERGVYKAMSEFDIFINYIESYMTTKM | |
| IMP-38 | YCVEYLESEEDKQQCGSNGASSSSPHMLRELRAAFGKVKTFFQMK | 246 |
| without signal | DQLNSMLLTQSLLDDFKGYLGCQALSEMIQFYLEEVMPQAENHGP | |
| peptide | DVKEHVNSLAEKLKTLRLRLRRCHRFLPCENKSKAVEQVKRVENM | |
| | LQERGVYKAMSEFDIFINYIESYMTTKS | |
| IMP-39 | YCVEYLESEEDKQQCGSNGASSSSPHMLRELRAAFGKVKTFFQMK | 247 |
| without signal | DQLNSMLLTQSLLDDFKGYLGCQALSEMIQFYLEEVMPQAENHGP | |
| peptide | DVKEHVNSLGEKLKTLRLRLRRCHRFLPCENKSKAVAQVKRVENM | |
| | LQERGVYKAMSEFDIFINYIESYMTTKS | |
| IMP-40 | YCVEYEESEEDRQQCSSSNFPASLPHMLRELRAAFGKVKTFFQMK | 248 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIES | |
| IMP-41 | YCVEYEESEEDRQQCSSSNFPASLPHMLRELRAAFGKVKTFFQMK | 249 |
| without signal | DQLNSMLLTQSLLDDFKGYLGCQAFSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIES | |
| IMP-42 | YCVEYLESREDEQQCSSSSNEPASLPHMLRELRAAFGKVKTFFQM | 250 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIES | |
| IMP-43 | VSNCGNLPHMLRDLRDAFSRVKTFFQMKDQLDNILLKESLLEDFK | 251 |
| without signal | GYLGCQALSEMIQFYLEEVMPQAENQDPHAKEHVNSLGENLKTLR | |
| peptide | LRLRRCHRFLPCENKSKAVEQVKNAFSKLQEKGVYKAMSEFDIFI | |
| | NYIEAYMTMKIRR | |
| IMP-44 | YCVEYLESREDEQQCGSSSNFPASLPHMLRELRAAFGKVKTFFQM | 252 |
| without signal | KDQLNSMLLTQSLLDDEKGYLGCQALSEMIQFYLEEVMPQAENHG | |
| peptide | PDIKEHVNSLGEKLKTLRLRLRRCHRFLPCENKSKAVEQVKRVEN | |
| | MLQERGVYKAMSEFDIFINYIES | |
| IMP-45 | YCVEYEESEEDRQQCSSSNFPASLPHMLRELRAAFGKVKTFFQMK | 253 |
| without signal | DQLNSMLLTQSLLDDFKGYLGCQALSEMIQFYLEEVMPQAENHGP | |
| peptide | DIKEHVNSPGEKLKTLRLRLRRCHRFLPCENKSKAVEQVKRVENM | |
| | LQERGVYKAMSEFDIFINYIES | |
| IMP-46 | YCTSCSYRDCTEDEDQKQQCEGGLRSLPHMLRELRAAFGKVKTFF | 254 |
| without signal | QMKDQLHSLLLTQSLLDDFKGYLGCQALSEMIQFYLEEVMPQAEN | |
| peptide | HGPDIKEHVNSLGEKLKTLRLRLRRCHRFLPCENKSKAVEKVKRV | |
| | FSELQERGVYKAMSEFDIFINYIETYMT | |
| IMP-47 | YCVEYEESEEDRQQCSSSNFPASLPHMPRELRAAFGKVKTFFQMK | 255 |
| without signal | DQLNSMLLTQSLLDDFKGYLGCQALSEMIQFYLEEVMPQAENHGP | |
| peptide | DIKEHVNSLGEKLKTLRLRLRRCHRFLPCENKSKAVEQVKRVENM | |
| | LQERGVYKAMSEFDIFINYIES | |
| IMP-48 | YCTSCSHRDCTEDDEQKQQCEGGSGGLGSLPHMLRELRAAFGKVK | 256 |
| without signal | TFFQMKDQLHSLLLTQSLLDDEKGYLGCQALSEMIQFYLEEVMPQ | |
| peptide | AENHGPDIKEHVNSLGEKLKTLRLRLRRCHRFLPCENKSKAVEKV | |
| | KRVFSELQERGVYKAMSEFDIFINYIETYMT | |
| IMP-49 | TDQCDNFPQMLRDLRDAFSRVKTFFQTKDEVDNLLLKESLLEDEK | 257 |
| without signal | GYLGCQALSEMIQFYLEEVMPQAENQDPGAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-50 | TDQCDNFPQMLRDLRDAFSRVKTFFQTKDEVDNLLLKESLLEDFK | 258 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-51 | TDQCDNFPQMLRDLRDAFSRVKTFFQTKDEVDNLLLKESLLEDEK | 259 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTMKAR | |
| IMP-52 | VSNCGNLPHMLRDLRDAFSRVKTFFQMKDQLDNILLKESLLEDFK | 260 |
| without signal | GYLGCQALSEMIQFYLEEVMPQAENQDPNAKEHVNSLGENLKTLR | |
| peptide | LRLRRCHRFLPCENKSKAVEQVKNAFSKLQEKGVYKAMSEEDIFI | |
| | NYIEAYMTMKTRR | |
| IMP-53 | TDQCDNFPQMLRDLRDAFSRVKTFFQTKDEVDNLLLKESLLEDEK | 261 |
| without signal | GYLGCQALSEMIQFYLEEVMPQAENQDPEAKDHVNSLGENLKTIR | |
| peptide | LRLRRCHRFLPCENKSKAVEQIKNAFNKLQEKGIYKAMSEFDIFI | |
| | NYIEAYMTIKAR | |
| IMP-54 | TDQCDNFPQMLRDLRDAFSRVKTFFQTKDEVDSLLLKESLLEDEK | 262 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-55 | TDQCDNFPQMLRDLRDAFSRVKTFFQTKDEVDNILLKESLLEDEK | 263 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-56 | TDQCDNFPQMLRDLRDAFSRVKTFFQTKDEVDNLELKESLLEDEK | 264 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-57 | TDQCDNFPQMLRDLRDAFSRVKTFFQTKDEVDNLLLKESLLEDEK | 265 |
| without signal | GYLGCQALSEMIQFYLEKVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-58 | TDQCDNFPQMLRDLRDAFSRVKTFFQTKDAVDNLLLKESLLEDEK | 266 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFDIFI | |
| | NYIEAYMTIKAR | |
| IMP-59 | ASNCGNLPHMLRDLRDAFSRVKTFFQMKDQLDNILLKESLLEDER | 267 |
| without signal | GYLGCQALSEMIQFYLEEVMPQAENQDPHSKEHVNSLGENLKTLR | |
| peptide | LRLRRCHRFLPCENKGKAVEQVKNAFSKLQEKGVYKAMSEFDIFI | |
| | NYIEAYMTMKLRR | |
| IMP-60 | TDQCDNFPQMLRDLRDAFSRVKTFFQTKDEVDNLLLKESLLEDEK | 268 |
| without signal | GYLGCQALSEMIQFYLEEVMPQAENQDPEAKDHVNSLGENLKTLR | |
| peptide | LRLRRCHRFLPCENKSKAVEQIKNAFNKLQEKGIYKAMSEFHIFI | |
| | NYIEAYMTIKAR | |
| IMP-61 | EMLRDLRDAFSRVKTFFQTKDEVDNLLLKESLLEDFKGYLGCQAL | 269 |
| without signal | SEMIQFYLEEVMPQAENQDPEAKDHVNSLGENLKTLRLRLRRCHR | |
| peptide | FLPCENKSKAVEQIKNAFNKLQEKGIYKAMSEFDIFINYIEAYMT | |
| | IKAR | |
| IMP-62 | ETCGNIPHMLRDLRDAFSRVKTFFQMKDQLDNILLKESLLEDEKG | 270 |
| without signal | YLGCQALSEMIQFYLEEVMPQAEAMSLKSQEHVNELGENLNTLRL | |
| peptide | RLRRCHRFLPCENKSKAVEQVKNAFSKLQEKGVYKAMSEFDIFIN | |
| | YIEAYMTMKLRR | |
| IMP-63 | TANNRAQKCFCEDGSNAGNSEETNTAAFQKKCDSEIPESLPYMLR | 271 |
| without signal | DLRNSSVQTRRYFQEKDEENSPLLTQKLLEDEKGYLGCQALSEMI | |
| peptide | QFYLEEVMPQAEDSNPSAKDSVTSLGEKLKTLRLRLRRCHRFLPC | |
| | ENKSKAVENLKSKFGDLGNQGVHKAMSEFDIFINYIETYMTTKMK | |
| IMP-64 | ATTTTIKNTKPQCRPEDYATRLQDLRVTFHRVKPTLQREDDYSVW | 272 |
| without signal | LDGTVVKGCWGCSVMDWLLRRYLETVEPAGDHVYPGLKTELHSMR | |
| peptide | STLESIYKDMRQCPLLGCGDKSVISRLSQEAERKSDNGTRKGLSE | |
| | LDTLESRLEEYLHSRK | |
| IMP-65 | ATTTTIKNTKPQCRPEDYATRLQDLRVTFHRVKPTLQREDDYSVW | 273 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-66 | ATTTTKNTKPQCRPEDYATRLQDLRVTFHRVKPTLQREDDYSVWL | 274 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-67 | ATTTTIKNTKPQCRPEDYATRLQDLRVTFHRVKPTLQREDDYSVW | 275 |
| without signal | LDGTMVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLESRLEEYLHSRK | |
| IMP-68 | ATTTTIKNTKPQCRPEDYATRLQDLRVTFHRVKSTLQREDDYSVW | 276 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLESRLEEYLHSRK | |
| IMP-69 | ATTTTIKNTKPQCRPEDYATRLQDLRVTFHRVKPTLQREDDYSVW | 277 |
| without signal | LDGTVVKGCWGCSVMDWLLRRYLEIVEPAGDHVYPGLKTELHSMR | |
| peptide | STLESIYKDMRQCPLLGCGDKSVISRLSQEAERKSDNGTRKGLSE | |
| | LDMLFSRLEEYLHSRK | |
| IMP-70 | ATTTTIKNTKPQCRPEDYATRLQDLRVTFHRVKPTLQREDDYSVW | 278 |
| without signal | LDGTVVKGCWGCSVMDWLLRRYLEIVLPAGDHVYPGLKTELHSMR | |
| peptide | STLESIYKDMRQCPLLGCGDKSVISRLSQEAERKSDNGTRKGLSE | |
| | LDTLESRLEEYLHSRK | |
| IMP-71 | AATTTIKNTKPQCRPEDYATRLQDLRVTFHRVKPTLQREDDYSVW | 279 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-72 | ATTTTINNTKPQCRPEDYATRLQDLRVTFHRVKPTLQREDDYSVW | 280 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-73 | ATTTTIKNTKPQCRPEDYATRLQDLRVTFHRVKPTLQREDDYSVW | 281 |
| without signal | LDGTVVKGCWGCSVMDWLLRRYLEIVEPAGDHVYPGLKTELHSMH | |
| peptide | STLESIYKDMRQCPLLGCGDKSVISRLSQEAERKSDNGTRKGLSE | |
| | LDTLFSRLEEYLHSRK | |
| IMP-74 | ATTTTIKNTKPQCRPEDYATRLQDLRVTFHRVKPTLQREDDYSVW | 282 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGMRKGLSE | |
| | LDTLESRLEEYLHSRK | |
| IMP-75 | ATTTIKNTKPQCRPEDYATRLQDLRVTFHRVKPTLQREDDYSVWL | 283 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-76 | ATTTTIKNTKPQCRPEDYATRLQDLRVTFDRVKPTLQREDDYSVW | 284 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-77 | AATTTIKNTKPQCRPEDYATRLQDLRVTFHRVKPTLQHEDDYSVW | 285 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-78 | ATTTTIKNTKPQCRPEDYATRLQDLRVTFHRVKPTLQREDDYSVW | 286 |
| without signal | LDGMVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLESRLEEYLHSRK | |
| IMP-79 | ATTTTIKNTKPQCRPEDYATRLQDLRVTFHRVKPTLQREDDYSVW | 287 |
| without signal | LDGTVVKGCWGCSVVDWLLRRYLEIVFPAGDHVYPGLKTELHSMR | |
| peptide | STLESIYKDMRQCPLLGCGDKSVISRLSQEAERKSDNGTRKGLSE | |
| | LDTLESRLEEYLHSRK | |
| IMP-80 | ATTTTIKNTKPQCRPEDYATRLQDLRITFHRVKPTLQREDDYSVW | 288 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLESRLEEYLHSRK | |
| IMP-81 | ATTTIKNTKPQCRPEDYATRLQDLRVTFHRIKPTLQREDDYSVWL | 289 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-82 | ATTTTIKNTKPQCRPEDYATRLQDLRVTFHRVKPTLQCEDDYSVW | 290 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLESRLEEYLHSRK | |
| IMP-83 | ATTTTTIKNTKPQCRPEDYATRLQDLRVTFHRVKPTLQREDDYSV | 291 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLESRLEEYLHSRK | |
| IMP-84 | ATTTTIKNTKPQCRPEDYATRLQDLRVTFHRVKPTLQREDDYSVW | 292 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDALFSRLEEYLHSRK | |
| IMP-85 | ATTTTIKNTKPQCRPEDYATRLQDLRVTFHRVKPTLQREDDYSVW | 293 |
| without signal | LDGTVVKGCWGCSVMDWLLRRYLEILFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-86 | ATTTTTIKNTKPQCRPEDYATRLQDLRVTFHRVKPTLQREDDYSV | 294 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGMRKGLS | |
| | ELDTLESRLEEYLHSRK | |
| IMP-87 | ATTTIKNTKPQCRPEDYATRLQDLRVTFHRVKPTLQREDDYSVWL | 295 |
| without signal | DGTVVKGCWGCSVMDWLLRRYLEIVFPAGDHVYPRLKTELHSMRS | |
| peptide | TLESIYKDMQQCPLLGCGDKSVISRLSQEAERKSDNGTRKGLSEL | |
| | DTLFSRLEEYLHSRK | |
| IMP-88 | AATTTTIKNTKPQCRPEDYATRLQDLRVTFHRVKPTLQREDDYSV | 296 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLESRLEEYLHSRK | |
| IMP-89 | AATTTTTIKNTKPQCRPEDYATRLQDLRVTFHRVKPTLQREDDYS | 297 |
| without signal | VWLDGTVVKGCWGCSVMDWLLRRYLEIVFPAGDHVYPGLKTELHS | |
| peptide | MRSTLESIYKDMRQCPLLGCGDKSVISRLSQEAEKKSDNGTRKGL | |
| | SELDTLESRLEEYLHSRK | |
| IMP-90 | AATTTTIKNTKPQCRPEDYASRLQDLRVTFHRVKPTLQREDDYSV | 298 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-91 | ATTTTTIKNTKPQCRPEDYATRLQDLRVTFHRVKPTLQREDDYSV | 299 |
| without signal | WLDGTVVKGCWGCSVMDWLLRRYLEIMFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-92 | ATTTIKNTKPRCRPEDYATRLQDLRVTFHRVKPTLQREDDYSVWL | 300 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-93 | ATTTTIKNTKPQCRPEDYATRLQDLRVTFHRVKPTLQREDDYSVW | 301 |
| without signal | LDGTVVKGCWGCSVMDWLLRRYLEIVFPAGDYVYPGLKTELHSMR | |
| peptide | STLESIYKDMRQCPLLGCGDKSVISRLSQEAERKSDNGMRKGLSE | |
| | LDTLFSRLEEYLHSRK | |
| IMP-94 | ATTTIKNTKPQCRPEDYATRLQDLRVTFHRVKPTLQREDDYSVWL | 302 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DALFSRLEEYLHSRK | |
| IMP-95 | ATTTTTIKNTKPRCRPEDYATRLQDLRVTFHRVKPTLQREDDYSV | 303 |
| without signal | WLDGTVVKGCWGCSVMDWLLRRYLEIVEPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-96 | AATTTTTIKNTKPQCRPEDYATRLQDLRVTFHRVKPTLQREDDYS | 304 |
| without signal | VWLDGTVVKGCWGCSVMDWLLRRYLEIVFPAGDHVYPGLKTELHS | |
| peptide | MRSTLESIYKDMRQCPLLGCGDKSVISRLSQEAERKSDNGTRKGL | |
| | SELDTLFSRLEEYLHSRK | |
| IMP-97 | ATTTTTTIKNTKPQCRPEDYATRLQDLRVTFHRVKPTLQREDDYS | 305 |
| without signal | VWLDGTVVKGCWGCSVMDWLLRRYLEIVFPAGDHVYPGLKTELHS | |
| peptide | MRSTLESIYKDMRQCPLLGCGDKSVISRLSQEAERKSDNGTRKGL | |
| | SELDTLESRLEEYLHSRK | |
| IMP-98 | AATTTIKNTKPQCRPEDYATRLQDLRVTFHRVKPTLQREDDYSVW | 306 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLNE | |
| | LDTLFSRLEEYLHSRK | |
| IMP-99 | ATTTTTIKNTKPQCRPEDYATRLQDLRVTFHRVKPTLQREDDYSV | 307 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDALFSRLEEYLHSRK | |
| IMP-100 | ATTTTIKNTKPQCRPEDYATRLQDLCVTFHRVKPTLQREDDYSVW | 308 |
| without signal | LDGTVVKGCWGCSVMDWLLRRYLEIVEPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-101 | ATTTTTTTIKNTKPQCRPEDYATRLQDLRVTFHRVKPTLQREDDY | 309 |
| without signal | SVWLDGTVVKGCWGCSVMDWLLRRYLEIVFPAGDHVYPGLKTELH | |
| peptide | SMRSTLESIYKDMRQCPLLGCGDKSVISRLSQKAERKSDNGTRKG | |
| | LSELDTLESRLEEYLHSRK | |
| IMP-102 | AATTTTIKNTKPQCRPEDYASRLQDLRVTFHRVKPTLQREDDYSV | 310 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKSLS | |
| | ELDTLFSRLEEYLHSRK | |
| IMP-103 | ATTTIKNTKPQCRPEDYATRLQDLRVTFHRVKPTLQREDDYSVWL | 311 |
| without signal | DGTVVKGCWGCSVMDWLLRRYLEIVFPAGDHVYSGLKTELHSMRS | |
| peptide | TLESIYKDMRQCPLLGCGDKSVISRLSQEAERKSDNGTRKGLSEL | |
| | DTLFSRLEEYLHSRK | |
| IMP-104 | ATTTTTTIKNTKPQCRPEDYATRLQDLRVTFHRVKPTLQREDDYS | 312 |
| without signal | VWLDGTVVKGCWGCSVMDWLLRRYLEIVFPAGDHVYPGLKTELHS | |
| peptide | MRSTLESIYKDMRQCPLLGCGDKSVISRLSQKAERKSDNGTRKGL | |
| | SELDTLESRLEEYLHSRK | |
| IMP-105 | ATTTTTMIKNTKPQCRPEDYATRLQDLRVTFHRVKPTLQREDDYS | 313 |
| without signal | VWLDGTVVKGCWGCSVMDWLLRRYLEIVEPAGDHVYPGLKTELHS | |
| peptide | MRSTLESIYKDMRQCPLLGCGDKSVISRLSQEAERKSDNGTRKGL | |
| | SELDTLFSRLEEYLHSRK | |
| IMP-106 | ATTTTTIKNTKPQCRPEDYATRLQDLRVTFHRVKPTLQREDDYSV | 314 |
| without signal | WLDGTVVKGCWGCSVMDWLLRRYLEIVFPAGDHVYPGLKTELHSM | |
| peptide | RSTLESIYKDMWQCPLLGCGDKSVISRLSQEAERKSDNGTRKGLS | |
| | ELDTLFSRLEEYLHSRK | |
| IMP-107 | AATTTTIKNTKPQCRPEDYATRLQDERVTFHRVKPTLQREDDYSV | 315 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-108 | ATIKNTKPQCRPEDYATRLQDLRVTFHRVKPTLQREDDYSVWLDG | 316 |
| without signal | TVVKGCWGCSVMDWLLRRYLEIVFPAGDHVYPGLKTELHSMRSTL | |
| peptide | ESIYKDMRQCPLLGCGDKSVISRLSQEAERKSDNGTRKGLSELDT | |
| | LFSRLEEYLHSRK | |
| IMP-109 | ATTTTTTTIKNTKPQCRPEDYATRLQDLRVTFHRVKPTLQREDDY | 317 |
| without signal | SVWLDGTVVKGCWGCSVMDWLLRRYLEIVFPAGDHVYPGLKTELH | |
| peptide | SMRSTLESIYKDMRQCPLLGCGDKSVISRLSQEAERKSDNGTRKG | |
| | LSELDTLFSRLEEYLHSRK | |
| IMP-110 | ATTTTTTMIKNTKPQCRPEDYATRLQDLRVTFHRVKPTLQREDDY | 318 |
| without signal | SVWLDGTVVKGCWGCSVMDWLLRRYLEIVFPAGDHVYPGLKTELH | |
| peptide | SMRSTLESIYKDMRQCPLLGCGDKSVISRLSQEAERKSDNGTRKG | |
| | LSELDTLFSRLEEYLHSRK | |
| IMP-111 | ATTTTTIKNTKPQCRPEDYATRLQDLRVTFHRVKPTLQREDDYSV | 319 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLR | |
| | ELDTLFSRLEEYLHSRK | |
| IMP-112 | AKPAATTTTTTTTIKNTKPQCRPEDYATRLQDLRVTFHRVKPTLQ | 320 |
| without signal | REDDYSVWLDGTVVKGCWGCSVMDWLLRRYLEIVFPAGDHVYPGL | |
| peptide | KTELHSMRSTLESIYKDMRQCPLLGCGDKSVISRLSQEAERKSDN | |
| | GTRKGLSELDTLFSRLEEYLHSRK | |
| IMP-113 | ATTTTTTIKNTKPQCRPEDYATRLQDLRVTFHRVKPTLQREDDYS | 321 |
| without signal | VWLDGTVVKGCWRCSVMDWLLRRYLEIVFPAGDHVYPGLKTELHS | |
| peptide | MRSTLESIYKDMRQCPLLGCGDKSVISRLSQEAERKSDNGTRKGL | |
| | SELDTLFSRLEEYLHSRK | |
| IMP-114 | ATTTTTIKNTKPQCRPEDYATRLQDLCVTFHRVKPTLQREDDYSV | 322 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLFSRLEEYLHSRK | |
| IMP-115 | ATTTTTTTTIKNTKPQCRPEDYATRLQDLRVTFHRVKPTLQREDD | 323 |
| without signal | YSVWLDGTVVKGCWGCSVMDWLLRRYLEIVFPAGDHVYPGLKTEL | |
| peptide | HSMRSTLESIYKDMRQCPLLGCGDKSVISRLSQEAERKSDNGTRK | |
| | GLSELDTLFSRLEEYLHSRK | |
| IMP-116 | ATTTTTTTIKNTKPQCRPEDYATRLQDLRVTFHRVKPTLQREDDY | 324 |
| without signal | SVWLDGTVVKGCWGCSVMDWLLRRYLEIVFPAGDHVYPGLKTELH | |
| peptide | SMRSTLESIYKDMRQCPLLGCGDKSVISRLSQEAERKSDNGTRKG | |
| | LSELDTLFIRLEEYLHSRK | |
| IMP-117 | ATTTTTIKNTKPQCRPEDYATRLQDLRVTFHRVKPTLQREDDYSV | 325 |
| without signal | WLDGTVVKGCWGRSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLESRLEEYLHSRK | |
| IMP-118 | ATTTTTIKNTKPQCRPEDYATRLQDLRVTFHRVKPTLQREDDYSV | 326 |
| without signal | WLDGTVVKGCWGCSVMDWLLRRYLEIVFPAGDHVYPGLKTELHSM | |
| peptide | RSTLESICKDMRQRPLLGCGDKSVISRLSQEAERKSDNGTRKGLS | |
| | ELDTLFSRLEEYLHSRK | |
| IMP-119 | AKPAATTTTTTTTTIKNTKPQCRPEDYATRLQDERVTFHRVKPTL | 327 |
| without signal | QREDDYSVWLDGTVVKGCWGCSVMDWLLRRYLEIVFPAGDHVYPG | |
| peptide | LKTELHSMRSTLESIYKDMRQCPLLGCGDKSVISRLSQEAERKSD | |
| | NGTRKGLSELDTLFSRLEEYLHSRK | |
| IMP-120 | ATIKNTKPQCRPEDYATRLQDLRVTFHRVKPTLPGHQREDDYSVW | 328 |
| without signal | LDGTVVKGCWGCSVMDWLLRRYLEIVFPAGDHVYPGLKTELHSMR | |
| peptide | STLESIYKDMRQCPLLGCGDKSVISRLSQEAERKSDNGTRKGLSE | |
| | LDTLFSRLEEYLHSRK | |
| IMP-121 | CAIASAKKCDDVSFDYILKDLRSEFSKIKSFVQDNDQENMMLLSQ | 329 |
| without signal | SMLDKLTSRIGCKSLSDMIKFYLNDVLPNAEKIEHMKNKITSIGE | |
| peptide | KLKSLKEKLISCDFLHCENHDEIKTVKTIFNKLKDKGIYKAMGEF | |
| | DIFINYLEKYIVKK | |
| IMP-122 | CAIASAKKCNDVSFDYILKDLRSEFSKIKSFVQDNDQENMMLLSQ | 330 |
| without signal | SMLDKLTSRIGCKSLSDMIKFYLNDVLPNAEKIEHMKNKITSIGE | |
| peptide | KLKSLKEKLISCDFLHCENHDEIKTVKTIFNKLKDKGIYKAMGEF | |
| | DIFINYLEKYIVKK | |
| IMP-123 | CVVASAKKCDDVSFDYILKDLRSEFSKIKSFVQDNDQENMMLLSQ | 331 |
| without signal | SMLDKLTSRIGCKSLSDMIKFYLNDVLPNAEKIEHMKNKITSIGE | |
| peptide | KLKSLKEKLISCDFLHCENHDEIKTVKTIFNKLKDKGIYKAMGEF | |
| | DIFINYLEKYIVKK | |
| IMP-124 | CVVAYAKKCDDVSFDYILKDLRSEFSKIKSFVQNNDQENMMLLSQ | 332 |
| without signal | SMLNKLTSCIGCKSLSDMIKFYLNDVLPNAEKIEQIKNIITSIGE | |
| peptide | KLKSLKEKLISCDFLHCENNDEIKTVKAIFNKLKDKGIYKAMGEF | |
| | DIFINYVEKYIVKT | |
| IMP-125 | CVVASAKKCDDVSFDYILKDLRSEFIKIKSFVQNNDQENMMLLSQ | 333 |
| without signal | SMLDKLTSCIGCKSLSDMIKFYLNDVLPNAEKIEQIKNIITSIGE | |
| peptide | KLKSLKEKLISCDFLHCENNDEIKTVKAIFNKLKDKGIYKAMGEF | |
| | DIFINYVEKYIVKT | |
| IMP-126 | KKCDDVSFDYILKDLRSEFSKIKSFVQNNDKENMMLLSQSMLDKL | 334 |
| without signal | TSCIGCKSLSDMIKFYLNDVLPNAEKIEHIKNKITSIGEKLKSLK | |
| peptide | EKLISCDFLHCENHDEIKAVKTIFNKLKDKGIYKAMGEFDIFINH | |
| | LEKYIVKK | |
| IMP-127 | CVVASAKKCDDVSFDYILKDLRSEFIKIKSFVQNNDQENMMLLSQ | 335 |
| without signal | SMLDKLTSRIGCKSLSDMIKFYLNDVLPNAEKIEQIKNIITSIGE | |
| peptide | KLKSLKEKLISCDFLHCENNDEIKTVKAIFNKLKDKGIYKAMGEF | |
| | DIFINYVEKYIVKT | |
| IMP-128 | CTVASAKKCDDVSFDYILKDLRSEFSKIKSFVQNNDKENMMLLSQ | 336 |
| without signal | SMLDKLTSCIGCKSLSDMIKFYLNDVLPNAEKIEHIKNKITSIGE | |
| peptide | KLKSLKEKLISCDFLHCENHDEIKAVKTIFNKLKDKGIYKAMGEF | |
| | DIFINHLEKYIVKK | |
| IMP-129 | ATTTTTIKNTKPQCRPEDYATRLQDLRVTFHRVKPTLDDYSVWLD | 337 |
| without signal | GTVVKGCWGCSVMDWLLRRYLEIVFPAGDHVYPGLKTELHSMRST | |
| peptide | LESIYKDMRQCPLLGCGDKAVISRLSQEAERKSDNGTRKGLSELD | |
| | TLFSRLEEYLHSRK | |
| IMP-130 | CTVASAKKCDDVSFDYILKDLRSEFSKIKSFVQNNDKENMMLLSQ | 338 |
| without signal | SMLDKLTRCIGCKSLSDMIKFYLNDVLPNAEKIEHIKNKITSIGE | |
| peptide | KLKSLKERLISCDFLHCENHDEIKAVKTIFNELKDKGIYKAMGEF | |
| | DIFINHLEKYIVKK | |
| IMP-131 | TDQCDNFPQMLRDLRDAFSRVKTFFQTKDEVDNLLLKESLLEDEK | 339 |
| without signal | GYLGCQALSEMIQFYLEEVMPQAENQDPEAKDHVNSLGENLKTLR | |
| peptide | LRLRRCHRFLPCENKSKAVEQIKMPLTSCRKKEFTKP | |
| IMP-132 | KGRDSKPSPACDPMHGALAGIFKELRTTYRSVRETLQTKDTVYYV | 340 |
| without signal | SLFHEQLLQEMLSPVGCRVTNELMQHYLDGVLPRAFHCGYDNTTL | |
| peptide | NALHELSSSLSTLYQHMLKCPALACTGQTPAWTQFLDTEHKLDPW | |
| | KGTVKATAEMDLLLNYLETELLQS | |
| IMP-133 | ATTTIKNTKPQCRPEDYATRLQDLRVTFHRVKPTLQREDDYSVWL | 341 |
| without signal | DGTVVKGCWGCSVMDWLLRRYLEIVFPAGDHVYPGLKTELHSMRS | |
| peptide | TLESIYKDMRQCEAERKSDNGTRKGLSELDTLFSRLEEYLHSRK | |
| IMP-134 | AHDHEHKVPPACDPVHGNLAGIFKELRTIYTSIREGLQKKDTVYY | 342 |
| without signal | TSLENDRVLQEMLSPMGCRVTNEIMEHYLDGVLPRASHLDYDNST | |
| peptide | LNGLHAFASSMQALYQHMLKCPALACTGKTPAWMYFLEVEHKLNP | |
| | WRGTAKAAAEADLLLNYLETELLQF | |
| IMP-135 | ASKPPVDCDPIHGTLSRIIKEVRTGYGSIKQALQSKDTVYYVSLF | 343 |
| without signal | HENLLNEMLSPVGCRVTNELMQHYLDGVLPRAFQCGYDNTTLDGL | |
| peptide | HSLVSSLDALYKHMLKCPALACTGQTPAWTQFLETEHKLDPWKGT | |
| | IKATAEMDLLVNYLETFLAQS | |
| IMP-136 | ATTTTTIKNTKPQCRPEDYATRLQDLRVTFHRVKPTLQREDDYSV | 344 |
| without signal | WLDGTVVKGCWGCSVMDWLLRRYLEIVFPAGDHVYPGLKTELHSM | |
| peptide | RSTLESIYKDMRQCPLLGCGDKSVISR | |
| IMP-137 | ATTTIKNTKPQCRPEDYATRLQDLRVTFHRVKPTLQREDDYSVWL | 345 |
| without signal | DGTVVKGCWGCSVMDWLLRRYLEIVFPAGDHVYPGLKTELHSMRS | |
| peptide | TLESIYKDMRQCVSVSVAALSAQR | |
| IMP-138 | IIDTCYDDQERERTKSNSISSVTPEMCKGLKQLVSTKLKDARQKE | 346 |
| without signal | KSVRDYFTSRDNDLDFMLLQGVKETHKKTCGCYVLYLLLSFYGKT | |
| peptide | IRDTIQSNKHKNLNTELTNLAVSVLSLEDLLEACGITCNPKKDSL | |
| | LKRIEEYMKEHGDDAIYKVIGEIEFLFQAIEKHVY | |
| IMP-139 | NSIIDMCYDDQERERTKSNSISSITPDMCKGLKQLVATKLKDARQ | 347 |
| without signal | KEKLVNSYFTSRDNDLTYMLLQGVRETHKKPCGCYVLYLLLTFYR | |
| peptide | KTIKDTIQSKKHESINTELTNLAVTVLSLEDLLEACGITCNPKKD | |
| | SLLKRIEGYTKEHGDDAIYKVIGEIDELFQAIERHVY | |
| IMP-140 | IIDTCYDDQERERTKSNSISSVTPEMCKGLKQLVATKLKDARQKE | 348 |
| without signal | KLVNDYFTGRDNDLSYMLLQGVRETHKKPCGCYVLYLLLSFYRKT | |
| peptide | IRDTIQSNKHASINAELTNLAVSVLSLEDLLDACGITCNPKKDSL | |
| | LKRIEEYMKEHGDDAIYKLIGEIEFLFQAIERHVYT | |
| IMP-141 | IIDTYDEDEDEDSIKLSSIGSITPEMCKNLKQLVASKLKDIRQKE | 349 |
| without signal | KSLRDYFTNLDDELDYMLLQGVGENHKKKCGCYILHLLLKFYSKT | |
| peptide | IRNTIQSEKHKNVNLELTNIAVSMLALEDLLEKCGITCNPKKDPL | |
| | LKRIEDYMKQHGDDGVNKAIAELEFLFQMIEKQVYI | |
| IMP-142 | AAQCRKGTITSRLKMLRTAFEKVREFYEDRDEEETALASTEHLHG | 350 |
| without signal | PESCSVIDELITHYTKCVIPAANEEEGADLLSLDTLQVALENVKG | |
| peptide | LLANCQEEFGCKPPFSMRDYKKQYRQLNKEKNAGMIKAMGELGML | |
| | ENGIEERVIGM | |
| IMP-143 | MYVQHGSDYCTTTVHADIASAISGMRAEYDSGLGHYFKSLVPHPD | 351 |
| without signal | NPYDTDDYKYMINNTNSYNCHALQSTINALLGMYGYVDIDEPHQL | |
| peptide | AMMKLATHTMQAAMLLNKCAKQLGCYHIPEDVETLHEAHPDDVMA | |
| | SLDTALNLMSMVTNEI | |
| IMP-144 | AAQCRKGTITSRLKMLRTAFEKVREFYEDSDEEETALASTEHLHG | 352 |
| without signal | PESCSVIDELITHYTKCVIPAANEEEGADLLSLDTLQVALENVKG | |
| peptide | LLANCQEEFGCKPPFSMRDYKKQYRQLNKEKNAGMIKAMGELGML | |
| | ENGIEERVIGM | |
| IMP-145 | ATIKCVGMSTLFNPELIQLRRLFGDGIKDFFQNKDEDLDNAFLNE | 353 |
| without signal | DVQRELASDCGCDHLMDMLSLYVNDTIPKGMKTEDAPSGLGQMGQ | |
| peptide | LMSSLYRKMDMCWSELGCSHNTRLTLQEYADKKGGWDNKALGESD | |
| | ILFDALELFFSKIK | |
| IMP-146 | MYVQHGSDYCTTTVRADIASAISGMRAEYNNGLGDYFKSLAPHPN | 354 |
| without signal | NPYDTDDYKYMINSTNSYNCHALQSTINALLGMYGYVDIDEPHQL | |
| peptide | AMMKLATHTMQTAMLLNKCAAQLGCYHIPFDVETLHEAHPNDVMA | |
| | SLDTALNLMSMVTNEI | |
| IMP-147 | MYARRSGDYCTTTVRADIASAISGMRAEYNSGLRDYFKSLVPHPD | 355 |
| without signal | NPYDTDDYKYMLNNTNSYNCHALQSTINALLGMYGYVDIDESHQL | |
| peptide | AMMKLATHTMQTAMMLNKCAAQLGCYHIPFDLETLHEAHPDDVMA | |
| | SLDTALNLMSMITNEI | |
| IMP-148 | MYVQHGSDYCTTTVRADIASAISGMRAEYNNGLGDYFKSLAPHPN | 356 |
| without signal | NPYDTDDYKYMINSTNSYNCHALQSTINALLGMYGYVDIDEPHQL | |
| peptide | AMMKLATHTMQTAMLLNKCAAQLGCYHIPEDVETLHEAHPDDVMA | |
| | SLDTALNLMSMVTNEI | |
| IMP-149 | NVHSGTEDNPCTNSKTVLNTLLNQIKQEYINNLLPYYKALTPKPV | 357 |
| without signal | DVFDDSYTYSIQSTDYNCYTIYETLNFLLGDVFPRATTDATVRLS | |
| peptide | LAKIATSSQQASMLMNLCKKELACGPAPFDMIKLYHDTKEYGADN | |
| | IMGTLDTPFQYFVIV | |
| IMP-150 | MYVQHGRGDYCTTTVRADIASAISGMRAEYDSGLGHYFKSLVPHP | 358 |
| without signal | DNPYDTDDYKYMINNTNSYNCHALQSTINALLGMYGYVDIDEPHQ | |
| peptide | LAMMKLATHTMQTAMLLNKCAEQLGCYHIPFDVEILHEAHPDDVM | |
| | ASLDTALNLMSMVTNEI | |
| IMP-151 | APATTPKDSCVYLIGQTPQLLRQLRNAYQAIIGADGSGVDEDDMP | 359 |
| without signal | IYPSDVMNELASTSVACDAIKKVLTMNIGILPNVTAAYPDKKSEV | |
| peptide | DEIGDNLSRLHQNIVNCRDELKCEDLPHWHQMAENYKEKPMQGES | |
| | EMDFVFQSVEKELVAKDVKNMKTKRKH | |
| IMP-152 | DDDPCTNVKTQLNTLFNQIKTEYDTNLKTYYQSIAPSAFDPENNT | 360 |
| without signal | NYLYSVQGNDYKCYTIFETLSFLMGDVYPRATTNESVRLSLAKVA | |
| peptide | TSSTQGAMVMNLCRQQLGCGPPPFDAKTLYDDRAEYGADDIMATL | |
| | DTALAKFKLVLESENVV | |
| IMP-153 | DDDPCTNVKTQLNTLFNQIKTEYDTNLKTYYQSIAPSAFDPENNT | 361 |
| without signal | NYLYSVQGNDYKCYTIFETLSFLMGDVYPRATTNESVRLSLAKVA | |
| peptide | TSSTQGAMVMNLCREQLGCGPPPFDAKTLYDDRAEYGADDIMATL | |
| | DTALAKFKLVLESENVV | |
| IMP-154 | RRRGDYCTTTVRADIASAISGMRAEYNSGLGDYFKSLVPHPDNPY | 362 |
| without signal | DTDDYKHMIDNANSYNCHALQSTINALLGMYGYVDIDEPHQLAMM | |
| peptide | KLATHTMQTAMLLNKCAAQLGCYHIPFDLETLREAPPADVMASLD | |
| | TALNLMSMITNEI | |
| IMP-155 | ASLSTHYNNYDLTRIATIDKDVCKRVAQHINDDFVNMRKLYETQL | 363 |
| without signal | KNYFQQLVPNPTDVFKDDSYMYMINGTDYNCHIIYETMRFLSGDV | |
| peptide | FPFATETEAELQYMWKMMLGVSQLSAYIGNCYQYFKCGPAPFDPQ | |
| | VLYHDRELFHADTVMAYLDTAFSHETL | |
| IMP-156 | LNCGIEHNELNNIKNIFFKVRNVVQADDVDHNLRILTPALLNNIT | 364 |
| without signal | VSETCFFIYDMFELYLNDVFVKYTNTALKLNILKSLSSVANNFLA | |
| peptide | IFNKVKKRRVKKNTVNVLEIKKLLLIDNNCKKLESEIDIFLTWVM | |
| | AKI | |
| IMP-157 | LNCGIEHNELNNIKNIFFKVRNVVQADDVDHNLRILTPALLNNIT | 365 |
| without signal | VSETCFFIYDMFELYLNDVFVKYTNTALKLNILKSLSSVANNFLA | |
| peptide | IFNKVKKRRVKKNNVNVLEIKKLLLIDNNCKKLFSEIDIFLTWVM | |
| | AKI | |
| IMP-158 | APATTPKDSCVYLIGQTPQLLRQLRNAYQAIIGADGSGVDEDDMP | 366 |
| without signal | IYPSDVMNELASTSVACDAIKKVLTMNIGILPNVTAAYPDKKSEV | |
| peptide | DEIGDNLSRLHQNIVNCVSRTQHLCYD | |
| IMP-159 | DNKYDSESGDDCPTLPTSLPHMLHELRAAFSRVKTFFQMKDQLDN | 367 |
| without signal | MLLDGSLLEDFKGYLGCQALSEMIQFYLEEVMPQAENHSPDQDKN | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-160 | DNKYDSESGNDCPTLPTSLPHMLHELRAAFSRVKTFFQMKDQLDN | 368 |
| without signal | MLLDGSLLEDFKGYLGCQALSEMIQFYLEEVMPQAENHSTGQEKD | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-161 | DNKYDSESGNDCPTLPTSLPHMLHELRAAFSRVKTFFQMKDQLDN | 369 |
| without signal | MLLDGSLLEDFKGYLGCQALSEMIQFYLEEVMPQAENHSTDQEKD | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-162 | DNKYDSESGDDCPTLPTSLPHMLHELRAAFSRVKTFFQMKDQLDN | 370 |
| without signal | MLLDGSLLEDFKGYLGCQALSEMIQFYLEEVMPQAENHSTGQEKD | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-163 | DNRYDGQDGNDCPTLPTSLPHMLHELRAAFSRVKTFFQMKDQLDN | 371 |
| without signal | MLLDGSLLEDFKGYLGCQALSEMIQFYLEEVMPQAENHSPDQDKN | |
| peptide | KVNSLGEKLKTLRVRLRRCHRFLPCENKSKAVEQVKSAFSKLQEK | |
| | GVYKAMSEFDIFINYIEAYMTTKMKN | |
| IMP-164 | ATTAIKNTKPQCRPEDYATRLQDLRVTFHRVKPTLQREDDYSVWL | 372 |
| without signal | DGTVVKGCWGCSVMDWLLRRYLEIVFPAGDHVYPGLKTELHSMRS | |
| peptide | TLESIYKDMRQCPLLGCGDKSVISRLSQEAERKSDNGTRKGLSEL | |
| | DTLFSRLEEYLHSRK | |
| IMP-165 | ATTTTIKNTKPQCRPEDYATRLQDLRVTFDRVKPTLQREDDYSVW | 373 |
| without signal | LDGTVVKGCWGCSVMDWLLRRYLEIVFPAGDHVYPGLKTELHSMR | |
| peptide | STLESIYKDMRQCPLLGCGDKSVISRLSQEAEKKSDNGTRKGLSE | |
| | LDTLFSRLEEYLHSRK | |
| IMP-166 | ATTTTTIKNTKPQCRPEDYATRLQDLRVTFYRVKPTLQREDDYSV | 374 |
| without signal | WLDGTVVKGCWGCSVMDWLLRRYLEIVFPAGDHVYPGLKTELHSM | |
| peptide | RSTLESIYKDMRQCPLLGCGDKSVISRLSQEAERKSDNGTRKGLS | |
| | ELDTLESRLEEYLHSRK | |
| IMP-167 | ATTTTTIKNTKPQCRPEDYATRLQDLRVTFHRVKPTLDDYSVWLD | 375 |
| without signal | GTVVKGCWGCSVMDWLLRRYLEIVFPAGDHVYPGLKTELHSMRST | |
| peptide | LESIYKDMRQCPLLGCGDKSVISRLSQEAERKSDNGTRKGLSELD | |
| | TLFSRLEEYLHSRK | |
| IMP-168 | STKKCDDVSFDYILKDLRSEFSKIKSFVQDNDQENMMLLSQSMLD | 376 |
| without signal | KLTSRIGCKSLSDMIKFYLNDVLPNAEKIEHMKNKITSIGEKLKS | |
| peptide | LKEKLISCDFLHCENHDEIKTVKTIENKLKDKGIYKAMGEFDIFI | |
| | NYLEKYIVKK | |
| IMP-169 | ATTTIKNTKPQCRPEDYATRLQDLRVTFHRVKPTLQREDDYSVWL | 377 |
| without signal | DGDHVYPGLKTELHSMRSTLESIYKDMRQCPLLGCGDKSVISRLS | |
| peptide | QEAERKSDNGTRKGLSELDTLFSRLEEYLHSRK | |
| IMP-170 | MIGTCYDEDEEIERLKSNSISSITPGMCRNLKHSVMIRLIDARQI | 378 |
| without signal | EASIRSYFTDGDNNLSFMLLQGIREISKKKCGCYILNLMLRFYIQ | |
| peptide | TIKHTILSNKHKDMNLELTNLAVTILSLESLLEKCGVTCNPVKDP | |
| | LLTRIEEYTRKHGDNAIYKTIGELEFLFDAIEKFV | |
| IMP-171 | QCRKGTITIRLKMLRTAFEKVREFYEDRDEEETALASTEHLHGPE | 379 |
| without signal | SCSVIDELITHYTKCVIPAANEEEGADLRSLDTLQFALENVKGLL | |
| peptide | ANCQEEFGCKPPFSMRDYKKQYRQLNKEKNAGMIKAMGELGMLFN | |
| | GIEERVIGM | |
| IMP-172 | QCRKGTITIRLKMLRTAFEKVREFYEDRDEEETALASTEHLHGPE | 380 |
| without signal | SCSVIDELITHYTKCVIPAANEEEGADLRSLDTLQFALENVKGLL | |
| peptide | ANCQEEFGCKPPFSMRDYKKQYRQLNKEKNAGMIKAMGELGMLEN | |
| | GIEERVNGM | |
| IMP-173 | QCRKGTITIRLKMLRTAFEKVREFYEDRDEEETALASTEHLHGPE | 381 |
| without signal | SCSVIDELITHYTKCVIPAANEEEGADLLSLDTLQFALENVKGLL | |
| peptide | ANCQEEFGCKPPFSMRDYKKQYRQLNKEKNAGMIKAMGELGMLEN | |
| | GIEERVIGM | |
| IMP-174 | AAQCRKGTITSRLKMLRTAFEKVREFYEDRDEEETALASTEHLHG | 382 |
| without signal | PESCSVIDELITHHTKCVIPAANEEEGADLLSLDTLQVALENVKG | |
| peptide | LLANCQEEFGCKPPFSMRDYKKQYRQLNKEKNAGMIKAMGELGML | |
| | ENGIEERVIGM | |
| IMP-175 | ATTTIKNTKPQCRPEDYATRLQDLRVTFHRVKPTLVGHVGDHVYP | 383 |
| without signal | GLKTELHSMRSTLESIYKDMRQCEAERKSDNGTRKGLSELDTLES | |
| peptide | RLEEYLHSRK | |
| IMP-273 | SPGQGTQSENSCTHEPGNLPNMLRDLRDAFSRVKTFFQMKDQLDN | 384 |
| with signal peptide | LLLKESLLEDFKGYLGCQALSEMIQFYLEEVMPQAENQDPDIKAH | |
| | VNSLGENLKTLRLRLRRCHRFLPCENKSKAVEQVKNAENKLQEKG | |
| | IYKAMSEFDIFINYIEAYMTMKIRN | |
| IMP-274 | SPGQGTQSENSCTHFPGYLPNMLRDLRDAFSRVKTFFQMKDQLDN | 385 |
| with signal peptide | LLLKESLLEDFKGYLGCQALSEMIQFYLEEVMPQAENQDPDIKAH | |
| | VQSLGENLKDLRLWLRRCHRFLPCENKSKAVEQVKNAFNKLQEKG | |
| | IYKAMSEFDIFINYIEAYMTMKIRN | |
| IMP-275 | SPGQGTQSENSCTHEPGNLPNMLRALRDAFSRVKTFFQMKDQLDN | 386 |
| with signal peptide | LLLKESLLEDFKGYLGCQALSEMIQFYLEEVMPQAENQDPDIKAH | |
| | VNSLGENLKTLRLRLRRCHRFLPCENKSKAVEQVKNAFNKLQEKG | |
| | IYKAMSEFDIFINYIEAYMTMKIRN | |

In some aspects and embodiments, the IL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, the IL-10R binding protein comprises an amino acid sequence at least 85% identical to the amino acid sequence of a protein set forth in Table 2. In some embodiments, the IL-10R binding protein comprises an amino acid sequence at least 90% identical to the amino acid sequence of a protein set forth in Table 2. In some embodiments, the IL-10R binding protein comprises an amino acid sequence at least 95% identical to the amino acid sequence of a protein set forth in Table 2. In some embodiments, the IL-10R binding protein comprises an amino acid sequence at least 99% identical to the amino acid sequence of a protein set forth in Table 2.

In some embodiments, the IL-10R binding protein comprises an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, the IL-10R binding protein comprises an amino acid sequence 85% identical to the amino acid sequence of a protein set forth in Table 2. In some embodiments, the IL-10R binding protein comprises an amino acid sequence 90% identical to the amino acid sequence of a protein set forth in Table 2. In some embodiments, the IL-10R binding protein comprises an amino acid sequence 95% identical to the amino acid sequence of a protein set forth in Table 2. In some embodiments, the IL-10R binding protein comprises an amino acid sequence 99% identical to the amino acid sequence of a protein set forth in Table 2. In some embodiments, the IL-10R binding protein comprises an amino acid sequence 100% identical to the amino acid sequence of a protein set forth in Table 2.

In some embodiments, the IL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, the IL-10R binding protein consists of an amino acid sequence at least 85% identical to the amino acid sequence of a protein set forth in Table 2. In some embodiments, the IL-10R binding protein consists of an amino acid sequence at least 90% identical to the amino acid sequence of a protein set forth in Table 2. In some embodiments, the IL-10R binding protein consists of an amino acid sequence at least 95% identical to the amino acid sequence of a protein set forth in Table 2. In some embodiments, the IL-10R binding protein consists of an amino acid sequence at least 99% identical to the amino acid sequence of a protein set forth in Table 2.

In some embodiments, the IL-10R binding protein consists of an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, the IL-10R binding protein consists of an amino acid sequence 85% identical to the amino acid sequence of a protein set forth in Table 2. In some embodiments, the IL-10R binding protein consists of an amino acid sequence 90% identical to the amino acid sequence of a protein set forth in Table 2. In some embodiments, the IL-10R binding protein consists of an amino acid sequence 95% identical to the amino acid sequence of a protein set forth in Table 2. In some embodiments, the IL-10R binding protein consists of an amino acid sequence 99% identical to the amino acid sequence of a protein set forth in Table 2. In some embodiments, the IL-10R binding protein consists of an amino acid sequence 100% identical to the amino acid sequence of a protein set forth in Table 2.

In some embodiments, the IL-10R binding protein comprises the amino acid sequence of a protein set forth in Table 2, and further comprising 1 or more but less than 15% (less than 12%, less than 10%, less than 8%), amino acid variations (e.g., substitutions, additions, deletions, etc.). In some embodiments, the IL-10R binding protein comprises the amino acid sequence of a protein set forth in Table 2, and further comprising at least 1, 2, 3, 4, 5, 6, 7, 8, 9, or 10 amino acid variations (e.g., substitutions, additions, deletions, etc.). In some embodiments, the IL-10R binding protein comprises the amino acid sequence of a protein set forth in Table 2, and further consisting of at least 1, 2, 3, 4, 5, 6, 7, 8, 9, or 10 amino acid variations (e.g., substitutions, additions, deletions, etc.). In some embodiments, the IL-10R binding protein comprises the amino acid sequence of a protein set forth in Table 2, and further comprising 1, 2, 3, 4, 5, 6, 7, 8, 9, or 10 amino acid variations (e.g., substitutions, additions, deletions, etc.). In some embodiments, the IL-10R binding protein comprises the amino acid sequence of a protein set forth in Table 2, and further consisting of 1, 2, 3, 4, 5, 6, 7, 8, 9, or 10 amino acid variations (e.g., substitutions, additions, deletions, etc.). In some embodiments, the IL-10R binding protein comprises the amino acid sequence of a protein set forth in Table 2, and further comprising or no more than 1, 2, 3, 4, 5, 6, 7, 8, 9, or 10 amino acid variations (e.g., substitutions, additions, deletions, etc.).

In some embodiments, the IL-10R binding protein consists of the amino acid sequence of a protein set forth in Table 2, and further comprising 1 or more but less than 15% (less than 12%, less than 10%, less than 8%), amino acid variations (e.g., substitutions, additions, deletions, etc.). In some embodiments, the IL-10R binding protein consists of the amino acid sequence of a protein set forth in Table 2, and further comprising at least 1, 2, 3, 4, 5, 6, 7, 8, 9, or 10 amino acid variations (e.g., substitutions, additions, deletions, etc.). In some embodiments, the IL-10R binding protein consists of the amino acid sequence of a protein set forth in Table 2, and further consisting of at least 1, 2, 3, 4, 5, 6, 7, 8, 9, or 10 amino acid variations (e.g., substitutions, additions, deletions, etc.). In some embodiments, the IL-10R binding protein consists of the amino acid sequence of a protein set forth in Table 2, and further comprising 1, 2, 3, 4, 5, 6, 7, 8, 9, or 10 amino acid variations (e.g., substitutions, additions, deletions, etc.). In some embodiments, the IL-10R binding protein consists of the amino acid sequence of a protein set forth in Table 2, and further consisting of 1, 2, 3, 4, 5, 6, 7, 8, 9, or 10 amino acid variations (e.g., substitutions, additions, deletions, etc.). In some embodiments, the IL-10R binding protein consists of the amino acid sequence of a protein set forth in Table 2, and further comprising or no more than 1, 2, 3, 4, 5, 6, 7, 8, 9, or 10 amino acid variations (e.g., substitutions, additions, deletions, etc.).

In some embodiments, the IL-10R binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, the IL-10R binding protein comprises an amino acid sequence at least 85% identical to the amino acid sequence set forth in any one of SEQ ID NOS: 34-386. In some embodiments, the IL-10R binding protein comprises an amino acid sequence at least 90% identical to the amino acid sequence set forth in any one of SEQ ID NOS: 34-386. In some embodiments, the IL-10R binding protein comprises an amino acid sequence at least 95% identical to the amino acid sequence set forth in any one of SEQ ID NOS: 34-386. In some embodiments, the IL-10R binding protein comprises an amino acid sequence at least 99% identical to the amino acid sequence set forth in any one of SEQ ID NOS: 34-386.

In some embodiments, the IL-10R binding protein comprises an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, the IL-10R binding protein comprises an amino acid sequence 85% identical to the amino acid sequence set forth in any one of SEQ ID NOS: 34-386. In some embodiments, the IL-10R binding protein comprises an amino acid sequence 90% identical to the amino acid sequence set forth in any one of SEQ ID NOS: 34-386. In some embodiments, the IL-10R binding protein comprises an amino acid sequence 95% identical to the amino acid sequence set forth in any one of SEQ ID NOS: 34-386. In some embodiments, the IL-10R binding protein comprises an amino acid sequence 99% identical to the amino acid sequence set forth in any one of SEQ ID NOS: 34-386. In some embodiments, the IL-10R binding protein comprises an amino acid sequence 100% identical to the amino acid sequence set forth in any one of SEQ ID NOS: 34-386.

In some embodiments, the IL-10R binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, the IL-10R binding protein consists of an amino acid sequence at least 85% identical to the amino acid sequence set forth in any one of SEQ ID NOS: 34-386. In some embodiments, the IL-10R binding protein consists of an amino acid sequence at least 90% identical to the amino acid sequence set forth in any one of SEQ ID NOS: 34-386. In some embodiments, the IL-10R binding protein consists of an amino acid sequence at least 95% identical to the amino acid sequence set forth in any one of SEQ ID NOS: 34-386. In some embodiments, the IL-10R binding protein consists of an amino acid sequence at least 99% identical to the amino acid sequence set forth in any one of SEQ ID NOS: 34-386. In some embodiments, the IL-10R binding protein consists of an amino acid sequence 100% identical to the amino acid sequence set forth in any one of SEQ ID NOS: 34-386.

In some embodiments, the IL-10R binding protein consists of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, the IL-10R binding protein consists of an amino acid sequence 85% identical to the amino acid sequence set forth in any one of SEQ ID NOS: 34-386. In some embodiments, the IL-10R binding protein consists of an amino acid sequence 90% identical to the amino acid sequence set forth in any one of SEQ ID NOS: 34-386. In some embodiments, the IL-10R binding protein consists of an amino acid sequence 95% identical to the amino acid sequence set forth in any one of SEQ ID NOS: 34-386. In some embodiments, the IL-10R binding protein consists of an amino acid sequence 100% identical to the amino acid sequence set forth in any one of SEQ ID NOS: 34-386.

In some embodiments, the IL-10R binding protein comprises the amino acid sequence set forth in any one of SEQ ID NOS: 34-386, and further comprising 1 or more but less than 15% (less than 12%, less than 10%, less than 8%), amino acid variations (e.g., substitutions, additions, deletions, etc.). In some embodiments, the IL-10R binding protein comprises the amino acid sequence set forth in any one of SEQ ID NOS: 34-386, and further comprising at least 1, 2, 3, 4, 5, 6, 7, 8, 9, or 10 amino acid variations (e.g., substitutions, additions, deletions, etc.). In some embodiments, the IL-10R binding protein comprises the amino acid sequence set forth in any one of SEQ ID NOS: 34-386, and further comprising 1, 2, 3, 4, 5, 6, 7, 8, 9, or 10 amino acid variations (e.g., substitutions, additions, deletions, etc.). In some embodiments, the IL-10R binding protein comprises the amino acid sequence set forth in any one of SEQ ID NOS: 34-386, and further consisting of 1, 2, 3, 4, 5, 6, 7, 8, 9, or 10 amino acid variations (e.g., substitutions, additions, deletions, etc.). In some embodiments, the IL-10R binding protein comprises the amino acid sequence set forth in any one of SEQ ID NOS: 34-386, and further comprising or consists of no more than 1, 2, 3, 4, 5, 6, 7, 8, 9, or 10 amino acid variations (e.g., substitutions, additions, deletions, etc.).

In some embodiments, the IL-10R binding protein consists of the amino acid sequence set forth in any one of SEQ ID NOS: 34-386, and further comprising 1 or more but less than 15% (less than 12%, less than 10%, less than 8%), amino acid variations (e.g., substitutions, additions, deletions, etc.). In some embodiments, the IL-10R binding protein consists of the amino acid sequence set forth in any one of SEQ ID NOS: 34-386, and further comprising at least 1, 2, 3, 4, 5, 6, 7, 8, 9, or 10 amino acid variations (e.g., substitutions, additions, deletions, etc.). In some embodiments, the IL-10R binding protein consists of the amino acid sequence set forth in any one of SEQ ID NOS: 34-386, and further comprising 1, 2, 3, 4, 5, 6, 7, 8, 9, or 10 amino acid variations (e.g., substitutions, additions, deletions, etc.). In some embodiments, the IL-10R binding protein consists of the amino acid sequence set forth in any one of SEQ ID NOS: 34-386, and further consisting of 1, 2, 3, 4, 5, 6, 7, 8, 9, or 10 amino acid variations (e.g., substitutions, additions, deletions, etc.). In some embodiments, the IL-10R binding protein consists of the amino acid sequence set forth in any one of SEQ ID NOS: 34-386, and further consisting of no more than 1, 2, 3, 4, 5, 6, 7, 8, 9, or 10 amino acid variations (e.g., substitutions, additions, deletions, etc.).

In some aspects and embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, a fusion protein described herein comprises a protein (or a functional fragment, functional variant, or functional fragment/variant thereof) comprising an amino acid sequence at least 85% identical to the amino acid sequence of a protein set forth in Table 2. In some embodiments, a fusion protein described herein comprises a protein (or a functional fragment, functional variant, or functional fragment/variant thereof) comprising an amino acid sequence at least 90% identical to the amino acid sequence of a protein set forth in Table 2. In some embodiments, a fusion protein described herein comprises a protein (or a functional fragment, functional variant, or functional fragment/variant thereof) comprising an amino acid sequence at least 95% identical to the amino acid sequence of a protein set forth in Table 2. In some embodiments, a fusion protein described herein comprises a protein (or a functional fragment, functional variant, or functional fragment/variant thereof) comprising an amino acid sequence at least 99% identical to the amino acid sequence of a protein set forth in Table 2.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, a fusion protein described herein comprises a protein (or a functional fragment, functional variant, or functional fragment/variant thereof) comprising an amino acid sequence 85% identical to the amino acid sequence of a protein set forth in Table 2. In some embodiments, a fusion protein described herein comprises a protein ((or a functional fragment, functional variant, or functional fragment/variant thereof) comprising an amino acid sequence 90% identical to the amino acid sequence of a protein set forth in Table 2. In some embodiments, a fusion protein described herein comprises a protein (or a functional fragment, functional variant, or functional fragment/variant thereof) comprising an amino acid sequence 95% identical to the amino acid sequence of a protein set forth in Table 2. In some embodiments, a fusion protein described herein comprises a protein (or a functional fragment, functional variant, or functional fragment/variant thereof) comprising an amino acid sequence 99% identical to the amino acid sequence of a protein set forth in Table 2. In some embodiments, a fusion protein described herein comprises a protein (or a functional fragment, functional variant, or functional fragment/variant thereof) comprising an amino acid sequence 100% identical to the amino acid sequence of a protein set forth in Table 2.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, a fusion protein described herein comprises a protein (or a functional fragment, functional variant, or functional fragment/variant thereof) consisting of an amino acid sequence at least 85% identical to the amino acid sequence of a protein set forth in Table 2. In some embodiments, a fusion protein described herein comprises a protein (or a functional fragment, functional variant, or functional fragment/variant thereof) consisting of an amino acid sequence at least 90% identical to the amino acid sequence of a protein set forth in Table 2. In some embodiments, a fusion protein described herein comprises a protein (or a functional fragment, functional variant, or functional fragment/variant thereof) consisting of an amino acid sequence at least 95% identical to the amino acid sequence of a protein set forth in Table 2. In some embodiments, a fusion protein described herein comprises a protein (or a functional fragment, functional variant, or functional fragment/variant thereof) consisting of an amino acid sequence at least 99% identical to the amino acid sequence of a protein set forth in Table 2.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 2. In some embodiments, a fusion protein described herein comprises a protein (or a functional fragment, functional variant, or functional fragment/variant thereof) consisting of an amino acid sequence 85% identical to the amino acid sequence of a protein set forth in Table 2. In some embodiments, a fusion protein described herein comprises a protein (or a functional fragment, functional variant, or functional fragment/variant thereof) consisting of an amino acid sequence 90% identical to the amino acid sequence of a protein set forth in Table 2. In some embodiments, a fusion protein described herein comprises a protein (or a functional fragment, functional variant, or functional fragment/variant thereof) consisting of an amino acid sequence 95% identical to the amino acid sequence of a protein set forth in Table 2. In some embodiments, a fusion protein described herein comprises a protein (or a functional fragment, functional variant, or functional fragment/variant thereof) consisting of an amino acid sequence 99% identical to the amino acid sequence of a protein set forth in Table 2. In some embodiments, a fusion protein described herein comprises a protein (or a functional fragment, functional variant, or functional fragment/variant thereof) consisting of an amino acid sequence 100% identical to the amino acid sequence of a protein set forth in Table 2.

In some embodiments, a fusion protein described herein comprises a protein (or a functional fragment, functional variant, or functional fragment/variant thereof) comprising the amino acid sequence of a protein set forth in Table 2, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 2, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 2, and further consisting of at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 2, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 2, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 2, and further comprising or no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence of a protein set forth in Table 2, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 2, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 2, and further consisting of at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consists of the amino acid sequence of a protein set forth in Table 2, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 2, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 2, and further comprising or no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence at least 85% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence at least 90% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence at least 95% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence at least 99% identical to the amino acid sequence set forth in any one of SEQ ID NOS: 34-386.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence 85% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence 90% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence 95% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence 99% identical to the amino acid sequence set forth in any one of SEQ ID NOS: 34-386. In some embodiments, a fusion protein described herein comprises a protein (or a functional fragment, functional variant, or functional fragment/variant thereof) comprising an amino acid sequence 100% identical to the amino acid sequence set forth in any one of SEQ ID NOS: 34-386.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence at least 85% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence at least 90% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence at least 95% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence at least 99% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence 100% identical to the amino acid sequence set forth in any one of SEQ ID NOS: 34-386.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence 85% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence 90% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence 95% identical to the amino acid sequence set forth in any one of SEQ ID NOS: 34-386. In some embodiments, a fusion protein described herein comprises a protein (or a functional fragment, functional variant, or functional fragment/variant thereof) consisting of an amino acid sequence 100% identical to the amino acid sequence set forth in any one of SEQ ID NOS: 34-386.

In some embodiments, a fusion protein described herein comprises a protein (or a functional fragment, functional variant, or functional fragment/variant thereof) comprising the amino acid sequence set forth in any one of SEQ ID NOS: 34-386, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 34-386, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 34-386, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 34-386, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 34-386, and further comprising or consists of no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence set forth in any one of SEQ ID NOS: 34-386, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 34-386, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 34-386, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 34-386, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 34-386, and further consisting of no more than 1, 2, 3, 4, 5, 6, 7, 8, 9, or 10 amino acid variations (e.g., substitutions, additions, deletions, etc.).

### (i) Exemplary Properties of IL-10R Binding Proteins

In some embodiments, the IL-10R binding proteins described herein are immunosuppressive (e.g., when administered to a subject). In some embodiments, the IL-10R binding proteins described herein are anti-inflammatory (e.g., when administered to a subject). In some embodiments, the IL-10R binding proteins described herein suppress pro-inflammatory response (e.g., when administered to a subject).

In some embodiments, the IL-10R binding protein specifically binds hIL-10Rα. In some embodiments, the IL-10R binding protein specifically binds hIL-10Rβ. In some embodiments, the IL-10R binding protein specifically binds both hIL-10Rα and hIL-10Rβ. In some embodiments, the IL-10R binding protein specifically binds hIL-10Rα and does not bind hIL-10Rβ. In some embodiments, the IL-10R binding protein specifically binds hIL-10Rβ and does not bind hIL-10Rα. In some embodiments, the IL-10R binding protein specifically binds both hIL-10Rα and hIL-10Rβ but binds hIL-10Rα with higher affinity than hIL-10Rβ. In some embodiments, the IL-10R binding protein specifically binds both hIL-10Rα and hIL-10Rβ but binds hIL-10Rβ with higher affinity than hIL-10Rα.

In some embodiments, the IL-10R binding protein is a hIL-10R agonist. In some embodiments, the IL-10R binding protein is a hIL-10Rα agonist. In some embodiments, the IL-10R binding protein is a hIL-10Rβ agonist. In some embodiments, the IL-10R binding protein is a hIL-10Rα agonist and a hIL-10Rβ agonist. In some embodiments, the IL-10R binding protein is capable of inducing or enhancing hIL-10R (e.g., hIL-10Rβ, hIL-10Rα) signaling.

### 5.2.1.2 TL1A Binding Proteins

In some aspects and embodiments, a fusion protein described herein (or combination regimen described herein) comprises a protein that specifically binds the hTL1A.

The amino acid sequence of proteins capable of specifically binding hTL1A is set forth in Table 3 (SEQ ID NOS: 387-454). The amino acid sequence of the mature form of the proteins (i.e., lacking the native signal peptide) is set forth in SEQ ID NOS: 387-420. The amino acid sequence of the immature form of the proteins (i.e., containing the native signal peptide) is set forth in SEQ ID NOS: 421-454.

The signal peptides have been computationally predicted using standard methods (see, e.g., Teufel 2022). A person of ordinary skill in the art would know how to experimentally identify and/or validate a computationally predicted signal peptide using standard methods known in the art, e.g., expression of a protein from a host cell and sequencing of the intracellular form and the extracellular form of the expressed protein (see, e.g., Zhang 2004).

TABLE 3
The Amino Acid Sequence of TL1A Binding Proteins.

| | | SEQ ID |
|---|---|---|
| Description | Amino Acid Sequence | NO |
| IMP-176 | TVYVVKSGVNKGKICDTCPPGTYKKRDCDRSLPTVCEPCGDGEYTS | 387 |
| without native | MNNSLPECLSCNHCYDPTEIEITPCNATTNTVCSCKEGYTFDSSIQ | |
| signal peptide | GCI | |
| IMP-177 | STYRVRSSGLTCSTCPPGTHKERDCSLNTETICKACGEGEYTAHNN | 388 |
| without native | SLPKCLACKSCFNATEIETKSCDPTSDTICACREGYSINNLGECN | |
| signal peptide | | |
| IMP-178 | STYRVRSSGLTCSMCPPGTYKERDCSLNTETLCKACGEGEYTAHKN | 389 |
| without native | SLPKCLACKSCFNATEIETKSCDPISDTICTCREGYSINNLGECN | |
| signal peptide | | |
| IMP-179 | RSKINSSLICDMCPPGYYKNKDCTSTSTTICLPCGEGEYTAYNNSL | 390 |
| without native | TKCIRCKDCYEENEKIFKPCNSTSDTICTCIDGYTKDETTDSCI | |
| signal peptide | | |
| IMP-180 | DTFTDEETGLVCEKCPPGTYREADCTETTKTVCKACGEGTFTDHPN | 391 |
| without native | NLPECKSCSTCDEATEVEVQKCTATANTVCACKDGFTVNEKGECV | |
| signal peptide | | |
| IMP-181 | DTYTDEKTGLTCEKCPPGTYREADCGENSKTVCKACGEGTYTDAPN | 392 |
| without native | HLPECKKCNTCDAETEVEVKACTATSDTVCACKEGFTVNENGECV | |
| signal peptide | | |
| IMP-182 | STYTDSATGRTCSKCPPGTYRSADCGATTSTVCTACGAGTYTDKDN | 393 |
| without native | NLSACLACSTCNAATETEVSACTATANTVCACKAGYTLDANGKCV | |
| signal peptide | | |
| IMP-183 | STYKDEKTGLTCQKCPPGTYRKADCGLFSKTECAACGEGTYTDKDN | 394 |
| without native | NLSKCLKCSTCNEETEVEVQKCTATADTVCACKEGFTKNEKGECQ | |
| signal peptide | | |
| IMP-184 | PTYRDEATGRVCQECPPGTYLEAHCGENTSTVCKACGEGTFTDKPN | 395 |
| without native | HLPQCLSCSTCEAATEVEVRACTATANTVCACREGFTRNEQGKCN | |
| signal peptide | | |
| IMP-185 | DTFTDEATGLTCAKCPPGTYREADCGLFSKTVCKACGEGTFTAEPN | 396 |
| without native | NLPTCKACNTCDAATEVEVQACTATADTVCACKEGFTKNENGECN | |
| signal peptide | | |
| IMP-186 | ATYTDTATGLTCAKCPPGTFRKADCGATTATVCEACGAGTYTAADN | 397 |
| without native | HLSACLACTTCDAATETETAACTATANTVCACKAGFTVDANGKCG | |
| signal peptide | | |
| IMP-187 | PTYTDTATGLTCAKCPPGTYRERDCGLESDTVCKACGEGTYTDTPN | 398 |
| without native | HLPKCLSCSTCDAATETEVQACTATANTVCACKDGFTVDSNGKCQ | |
| signal peptide | | |
| IMP-188 | DTYTDTATGLTCAKCPPGTYREADCGLFSETVCKACGAGTYTDKPN | 399 |
| without native | HLPACLACGTCDAATEVEVQACTATANTVCACKAGFTKNEKGECV | |
| signal peptide | | |
| IMP-189 | PTYTDTATGLTCEQCPPGTYRRADCGATTATVCAACGAGTYTSAPN | 400 |
| without native | NLPTCLACPTCDAATEVEVQPCVATANTVCACRAGFTVDAQGRCQ | |
| signal peptide | | |
| IMP-190 | ATYTDTATGLTCTKCPPGTHRKADCGLLTSTVCEACGEGTYTSEDN | 401 |
| without native | NLSSCLSCSTCNEATETEVQACTATSDTVCECKEGFTVDANGKCG | |
| signal peptide | | |
| IMP-191 | ATYKDEKTGLTCEKCPPGTYREADCTETTKTVCKACGEGTYTDKDN | 402 |
| without native | NLPKCLECNTCDEKTEVEVQKCTATSNTVCACKEGFTKNEKGECK | |
| signal peptide | | |
| IMP-192 | KTYKDPETGLTCEKCPPGTYLEANCTPTSPTVCKPCGEGTYTEEEN | 403 |
| without native | ALTACKPCSSCDPATEEVVQPCTPTSDTVCSCKPGYSFNSNGLCV | |
| signal peptide | | |
| IMP-193 | ETYTDSSTGLTCAKCPPGYYLSQTCTETSPTVCKACGSGTYTTTPN | 404 |
| without native | ALTSCKACSTCDSSTEVTTKSCTPTSDTVCSCKSGYSKNSSGKCV | |
| signal peptide | | |
| IMP-194 | QTYTDPATGLTCDLCPPGTYLEAACTATSPTVCKPCPSGTYTTAPN | 405 |
| without native | ALTSCRKCSTCNPATEVTTQPCTPTSDTVCSCKSGYTLDSNGQCV | |
| signal peptide | | |
| IMP-195 | KTYVDPTTGLTCDMCPPGTYLKKTCSLNSRTVCKPCGKGTYTSTYN | 406 |
| without native | NLKKCKKCSTCDPRTEVVSQPCTQTSDTVCECRPGYSRDSNGRCV | |
| signal peptide | | |
| IMP-196 | ATYTDPETGLTCEKCPPGTYLEAPCTETTPTVCKPCGEGEYTTVPN | 407 |
| without native | ALRKCRECTTCNPATEEVSADCTPTSNTVCTCKPGYVKDANGKCV | |
| signal peptide | | |
| IMP-197 | QTYVDPATGLTCTLCPPGTYLKKPCTATSPTVCKPCGSGTYTSKPN | 408 |
| without native | ALTKCQECTTCDPATERVVRPCTPTHNTVCECKPGYKRNSKGQCV | |
| signal peptide | | |
| IMP-198 | ATYTDPATGLVCELCPPGSYLAAPCTATTPTVCKPCPEGYYTTEPN | 409 |
| without native | ALPRCLPCTTCNPETEVVIEPCTPTSDTVCECKPGYSLNENGECV | |
| signal peptide | | |
| IMP-199 | DTYTDPTTGLTCKKCPPGTYLKKNCTATSPTVCKKCGPGQYTTKYN | 410 |
| without native | NLKKCNKCSTCDPTTEEVSQACTPTHDTVCKCKPGYSLNSKGRCV | |
| signal peptide | | |
| IMP-200 | STYTNPSTGLTCSMCPPGTYLERDCSSSSGTVCKPCGPGTYTTAYN | 411 |
| without native | NLKSCKKCTKCDPKTEKVVKECTPTSNTVCECKPGFKFDSNGKCR | |
| signal peptide | | |
| IMP-201 | KTFVDDKTGLTCEQCPPGTYLESTCSETSPTVCKPCGPGEYTTEYN | 412 |
| without native | NLTKCKKCSTCDPATEVVVSACTPTSDTVCACKPGFSFNEEGKCV | |
| signal peptide | | |
| IMP-202 | STYVNPTTGLTCSKCPPGTYLKASCTATSKTVCTPCGSGTYTTTAN | 413 |
| without native | NLSSCKQCSTCDPATETVSQPCTPTSDTVCSCKSGYTFDSNGKCV | |
| signal peptide | | |
| IMP-203 | ETYKDPSTGLLCEKCPPGTYLSSPCTTTSGTVCKPCGSGTYTTQPN | 414 |
| without native | ALSSCSSCSTCDSATQTVSKACTSTSDTVCSCKSGYSLDSNGNCV | |
| signal peptide | | |
| IMP-204 | ETYTDPATGRVCEKCPPGTYVAAPCTATSPTVCRPCGPGTYTSAPN | 415 |
| without native | ALTSCLACTSCDPATEAVSQPCTPTANTVCVCKPGFSFNANGQCV | |
| signal peptide | | |
| IMP-205 | ETYTNASTGLTCNKCPPGTYLAKDCTSTSDTVCQDCGPGTYTTSPN | 416 |
| without native | AKSSCNACSTCDPATEEVITPCTPTSDTVCACKPGFTWNQNGQCV | |
| signal peptide | | |
| IMP-206 | ETYTDPATGLTCQKCPPGTYLKAPCTSTSPTVCTPCGSGYYTSSSN | 417 |
| without native | NLTSCSKCNTCDPSTESVSSACTPTSNTVCSCKSGYTLDSNGNCV | |
| signal peptide | | |
| IMP-207 | MSTYNTKTSPSLKCDMCPPGYYKHEDCTSNTKTVCSPCGEGEYTAY | 418 |
| without native | NNSLTKCLRCSDCYGENEITTKQCTNTSNTVCECMDGYTKDETIDA | |
| signal peptide | CIK | |
| IMP-208 | ASTYTSKIDASLICDMCPPGSYKYKDCTYDSKTVCLPCGDGEYTSY | 419 |
| without native | NNSLAKCLRCDDCYDENEVTAKPCDSTSNTICKCMDGYTKDNTISA | |
| signal peptide | CVKTSHIT | |
| IMP-209 | KVPTWTDPVTGLTCDRCPAGTHVVKQCTATTPTECGACPANHYTEF | 420 |
| without native | WNYLNRCLYCGVRCTEQQVEKSTCSATHNRVCECKPGYHIYADDFC | |
| signal peptide | LRHSTCPPGQGLLVAGTPTSNTRCGPCQAGYFSAEDSTEACKPHTP | |
| | CKDTERSVPGTATQDTFCTSCQARKCDITAPPAPDKAVCDDAFIDF | |
| | VERYPLNAKKAKNLMRKLRKMGKGKTIRESFQAIMAKRNDQPLACE | |
| | VLSTLNDVDSDMGCRIKTFFLGWNEDEC | |
| IMP-176 | MIITLLALLLVVSTESSTVYVVKSGVNKGKICDTCPPGTYKKRDCD | 421 |
| with native signal | RSLPTVCEPCGDGEYTSMNNSLPECLSCNHCYDPTEIEITPCNATT | |
| peptide | NTVCSCKEGYTFDSSIQGCI | |
| IMP-177 | MGKNSLVLTIGVLMTLITLRSSFSASTYRVRSSGLTCSTCPPGTHK | 422 |
| with native signal | ERDCSLNTETICKACGEGEYTAHNNSLPKCLACKSCFNATEIETKS | |
| peptide | CDPTSDTICACREGYSINNLGECN | |
| IMP-178 | MGKNSLVLMIGVLMTLITLRSSFSASTYRVRSSGLTCSMCPPGTYK | 423 |
| with native signal | ERDCSLNTETLCKACGEGEYTAHKNSLPKCLACKSCFNATEIETKS | |
| peptide | CDPISDTICTCREGYSINNLGECN | |
| IMP-179 | MHYKNKMSVNILIITVLIGISFQASTYRSKINSSLICDMCPPGYYK | 424 |
| with native signal | NKDCTSTSTTICLPCGEGEYTAYNNSLTKCIRCKDCYEENEKIFKP | |
| peptide | CNSTSDTICTCIDGYTKDETTDSCI | |
| IMP-180 | MGKNSLVLTIGVLMTLITLRSSFSADTFTDEETGLVCEKCPPGTYR | 425 |
| with native signal | EADCTETTKTVCKACGEGTFTDHPNNLPECKSCSTCDEATEVEVQK | |
| peptide | CTATANTVCACKDGFTVNEKGECV | |
| IMP-181 | MGKNSLVLTIGVLMTLITLRSSFSADTYTDEKTGLTCEKCPPGTYR | 426 |
| with native signal | EADCGENSKTVCKACGEGTYTDAPNHLPECKKCNTCDAETEVEVKA | |
| peptide | CTATSDTVCACKEGFTVNENGECV | |
| IMP-182 | MGKNSLVLTIGVLMTLITLRSSFSASTYTDSATGRTCSKCPPGTYR | 427 |
| with native signal | SADCGATTSTVCTACGAGTYTDKDNNLSACLACSTCNAATETEVSA | |
| peptide | CTATANTVCACKAGYTLDANGKCV | |
| IMP-183 | MGKNSLVLTIGVLMTLITLRSSFSASTYKDEKTGLTCQKCPPGTYR | 428 |
| with native signal | KADCGLFSKTECAACGEGTYTDKDNNLSKCLKCSTCNEETEVEVQK | |
| peptide | CTATADTVCACKEGFTKNEKGECQ | |
| IMP-184 | MGKNSLVLTIGVLMTLITLRSSFSAPTYRDEATGRVCQECPPGTYL | 429 |
| with native signal | EAHCGENTSTVCKACGEGTFTDKPNHLPQCLSCSTCEAATEVEVRA | |
| peptide | CTATANTVCACREGFTRNEQGKCN | |
| IMP-185 | MGKNSLVLTIGVLMTLITLRSSFSADTFTDEATGLTCAKCPPGTYR | 430 |
| with native signal | EADCGLFSKTVCKACGEGTFTAEPNNLPTCKACNTCDAATEVEVQA | |
| peptide | CTATADTVCACKEGFTKNENGECN | |
| IMP-186 | MGKNSLVLTIGVLMTLITLRSSFSAATYTDTATGLTCAKCPPGTFR | 431 |
| with native signal | KADCGATTATVCEACGAGTYTAADNHLSACLACTTCDAATETETAA | |
| peptide | CTATANTVCACKAGFTVDANGKCG | |
| IMP-187 | MGKNSLVLTIGVLMTLITLRSSFSAPTYTDTATGLTCAKCPPGTYR | 432 |
| with native signal | ERDCGLFSDTVCKACGEGTYTDTPNHLPKCLSCSTCDAATETEVQA | |
| peptide | CTATANTVCACKDGFTVDSNGKCQ | |
| IMP-188 | MGKNSLVLTIGVLMTLITLRSSFSADTYTDTATGLTCAKCPPGTYR | 433 |
| with native signal | EADCGLFSETVCKACGAGTYTDKPNHLPACLACGTCDAATEVEVQA | |
| peptide | CTATANTVCACKAGFTKNEKGECV | |
| IMP-189 | MGKNSLVLTIGVLMTLITLRSSFSAPTYTDTATGLTCEQCPPGTYR | 434 |
| with native signal | RADCGATTATVCAACGAGTYTSAPNNLPTCLACPTCDAATEVEVQP | |
| peptide | CVATANTVCACRAGFTVDAQGRCQ | |
| IMP-190 | MGKNSLVLTIGVLMTLITLRSSFSAATYTDTATGLTCTKCPPGTHR | 435 |
| with native signal | KADCGLLTSTVCEACGEGTYTSEDNNLSSCLSCSTCNEATETEVQA | |
| peptide | CTATSDTVCECKEGFTVDANGKCG | |
| IMP-191 | MGKNSLVLTIGVLMTLITLRSSFSAATYKDEKTGLTCEKCPPGTYR | 436 |
| with native signal | EADCTETTKTVCKACGEGTYTDKDNNLPKCLECNTCDEKTEVEVQK | |
| peptide | CTATSNTVCACKEGFTKNEKGECK | |
| IMP-192 | MGKNSLVLTIGVLMTLITLRSSFSAKTYKDPETGLTCEKCPPGTYL | 437 |
| with native signal | EANCTPTSPTVCKPCGEGTYTEEENALTACKPCSSCDPATEEVVQP | |
| peptide | CTPTSDTVCSCKPGYSFNSNGLCV | |
| IMP-193 | MGKNSLVLTIGVLMTLITLRSSFSAETYTDSSTGLTCAKCPPGYYL | 438 |
| with native signal | SQTCTETSPTVCKACGSGTYTTTPNALTSCKACSTCDSSTEVTTKS | |
| peptide | CTPTSDTVCSCKSGYSKNSSGKCV | |
| IMP-194 | MGKNSLVLTIGVLMTLITLRSSFSAQTYTDPATGLTCDLCPPGTYL | 439 |
| with native signal | EAACTATSPTVCKPCPSGTYTTAPNALTSCRKCSTCNPATEVTTQP | |
| peptide | CTPTSDTVCSCKSGYTLDSNGQCV | |
| IMP-195 | MGKNSLVLTIGVLMTLITLRSSFSAKTYVDPTTGLTCDMCPPGTYL | 440 |
| with native signal | KKTCSLNSRTVCKPCGKGTYTSTYNNLKKCKKCSTCDPRTEVVSQP | |
| peptide | CTQTSDTVCECRPGYSRDSNGRCV | |
| IMP-196 | MGKNSLVLTIGVLMTLITLRSSFSAATYTDPETGLTCEKCPPGTYL | 441 |
| with native signal | EAPCTETTPTVCKPCGEGEYTTVPNALRKCRECTTCNPATEEVSAD | |
| peptide | CTPTSNTVCTCKPGYVKDANGKCV | |
| IMP-197 | MGKNSLVLTIGVLMTLITLRSSFSAQTYVDPATGLTCTLCPPGTYL | 442 |
| with native signal | KKPCTATSPTVCKPCGSGTYTSKPNALTKCQECTTCDPATERVVRP | |
| peptide | CTPTHNTVCECKPGYKRNSKGQCV | |
| IMP-198 | MGKNSLVLTIGVLMTLITLRSSFSAATYTDPATGLVCELCPPGSYL | 443 |
| with native signal | AAPCTATTPTVCKPCPEGYYTTEPNALPRCLPCTTCNPETEVVIEP | |
| peptide | CTPTSDTVCECKPGYSLNENGECV | |
| IMP-199 | MGKNSLVLTIGVLMTLITLRSSFSADTYTDPTTGLTCKKCPPGTYL | 444 |
| with native signal | KKNCTATSPTVCKKCGPGQYTTKYNNLKKCNKCSTCDPTTEEVSQA | |
| peptide | CTPTHDTVCKCKPGYSLNSKGRCV | |
| IMP-200 | MGKNSLVLTIGVLMTLITLRSSFSASTYTNPSTGLTCSMCPPGTYL | 445 |
| with native signal | ERDCSSSSGTVCKPCGPGTYTTAYNNLKSCKKCTKCDPKTEKVVKE | |
| peptide | CTPTSNTVCECKPGFKFDSNGKCR | |
| IMP-201 | MGKNSLVLTIGVLMTLITLRSSFSAKTFVDDKTGLTCEQCPPGTYL | 446 |
| with native signal | ESTCSETSPTVCKPCGPGEYTTEYNNLTKCKKCSTCDPATEVVVSA | |
| peptide | CTPTSDTVCACKPGFSFNEEGKCV | |
| IMP-202 | MGKNSLVLTIGVLMTLITLRSSFSASTYVNPTTGLTCSKCPPGTYL | 447 |
| with native signal | KASCTATSKTVCTPCGSGTYTTTANNLSSCKQCSTCDPATETVSQP | |
| peptide | CTPTSDTVCSCKSGYTFDSNGKCV | |
| IMP-203 | MGKNSLVLTIGVLMTLITLRSSFSAETYKDPSTGLLCEKCPPGTYL | 448 |
| with native signal | SSPCTTTSGTVCKPCGSGTYTTQPNALSSCSSCSTCDSATQTVSKA | |
| peptide | CTSTSDTVCSCKSGYSLDSNGNCV | |
| IMP-204 | MGKNSLVLTIGVLMTLITLRSSFSAETYTDPATGRVCEKCPPGTYV | 449 |
| with native signal | AAPCTATSPTVCRPCGPGTYTSAPNALTSCLACTSCDPATEAVSQP | |
| peptide | CTPTANTVCVCKPGFSFNANGQCV | |
| IMP-205 | MGKNSLVLTIGVLMTLITLRSSFSAETYTNASTGLTCNKCPPGTYL | 450 |
| with native signal | AKDCTSTSDTVCQDCGPGTYTTSPNAKSSCNACSTCDPATEEVITP | |
| peptide | CTPTSDTVCACKPGFTWNQNGQCV | |
| IMP-206 | MGKNSLVLTIGVLMTLITLRSSFSAETYTDPATGLTCQKCPPGTYL | 451 |
| with native signal | KAPCTSTSPTVCTPCGSGYYTSSSNNLTSCSKCNTCDPSTESVSSA | |
| peptide | CTPTSNTVCSCKSGYTLDSNGNCV | |
| IMP-207 | MSNNLCQIITICIITMVCISYQMSTYNTKTSPSLKCDMCPPGYYKH | 452 |
| with native signal | EDCTSNTKTVCSPCGEGEYTAYNNSLTKCLRCSDCYGENEITTKQC | |
| peptide | TNTSNTVCECMDGYTKDETIDACIK | |
| IMP-208 | MTNYLYISANIFVIILLAEITFQASTYTSKIDASLICDMCPPGSYK | 453 |
| with native signal | YKDCTYDSKTVCLPCGDGEYTSYNNSLAKCLRCDDCYDENEVTAKP | |
| peptide | CDSTSNTICKCMDGYTKDNTISACVKTSHIT | |
| IMP-209 | MFRGTLMVLACVAACFSEKVPTWTDPVTGLTCDRCPAGTHVVKQCT | 454 |
| with native signal | ATTPTECGACPANHYTEFWNYLNRCLYCGVRCTEQQVEKSTCSATH | |
| peptide | NRVCECKPGYHIYADDFCLRHSTCPPGQGLLVAGTPTSNTRCGPCQ | |
| | AGYFSAEDSTEACKPHTPCKDTERSVPGTATQDTFCTSCQARKCDI | |
| | TAPPAPDKAVCDDAFIDEVERYPLNAKKAKNLMRKLRKMGKGKTIR | |
| | ESFQAIMAKRNDQPLACEVLSTLNDVDSDMGCRIKTFFLGWNEDEC | |

In some aspects and embodiments, the TL1A binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 3. In some embodiments, the TL1A binding protein comprises an amino acid sequence at least 85% identical to the amino acid sequence of a protein set forth in Table 3. In some embodiments, the TL1A binding protein comprises an amino acid sequence at least 90% identical to the amino acid sequence of a protein set forth in Table 3. In some embodiments, the TL1A binding protein comprises an amino acid sequence at least 95% identical to the amino acid sequence of a protein set forth in Table 3. In some embodiments, the TL1A binding protein comprises an amino acid sequence at least 99% identical to the amino acid sequence of a protein set forth in Table 3.

In some embodiments, the TL1A binding protein comprises an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 3. In some embodiments, the TL1A binding protein comprises an amino acid sequence 85% identical to the amino acid sequence of a protein set forth in Table 3. In some embodiments, the TL1A binding protein comprises an amino acid sequence 90% identical to the amino acid sequence of a protein set forth in Table 3. In some embodiments, the TL1A binding protein comprises an amino acid sequence 95% identical to the amino acid sequence of a protein set forth in Table 3. In some embodiments, the TL1A binding protein comprises an amino acid sequence 99% identical to the amino acid sequence of a protein set forth in Table 3. In some embodiments, the TL1A binding protein comprises an amino acid sequence 100% identical to the amino acid sequence of a protein set forth in Table 3.

In some embodiments, the TL1A binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 3. In some embodiments, the TL1A binding protein consists of an amino acid sequence at least 85% identical to the amino acid sequence of a protein set forth in Table 3. In some embodiments, the TL1A binding protein consists of an amino acid sequence at least 90% identical to the amino acid sequence of a protein set forth in Table 3. In some embodiments, the TL1A binding protein consists of an amino acid sequence at least 95% identical to the amino acid sequence of a protein set forth in Table 3. In some embodiments, the TL1A binding protein consists of an amino acid sequence at least 99% identical to the amino acid sequence of a protein set forth in Table 3.

In some embodiments, the TL1A binding protein consists of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, the TL1A binding protein consists of an amino acid sequence 85% identical to the amino acid sequence set forth in any one of SEQ ID NOS: 387-454. In some embodiments, the TL1A binding protein consists of an amino acid sequence 90% identical to the amino acid sequence set forth in any one of SEQ ID NOS: 387-454. In some embodiments, the TL1A binding protein consists of an amino acid sequence 95% identical to the amino acid sequence set forth in any one of SEQ ID NOS: 387-454. In some embodiments, the TL1A binding protein consists of an amino acid sequence 99% identical to the amino acid sequence set forth in any one of SEQ ID NOS: 387-454. In some embodiments, the TL1A binding protein consists of an amino acid sequence 100% identical to the amino acid sequence set forth in any one of SEQ ID NOS: 387-454.

In some embodiments, the TL1A binding protein consists of an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 3. In some embodiments, the TL1A binding protein consists of an amino acid sequence 85% identical to the amino acid sequence of a protein set forth in Table 3. In some embodiments, the TL1A binding protein consists of an amino acid sequence 90% identical to the amino acid sequence of a protein set forth in Table 3. In some embodiments, the TL1A binding protein consists of an amino acid sequence 95% identical to the amino acid sequence of a protein set forth in Table 3. In some embodiments, the TL1A binding protein consists of an amino acid sequence 99% identical to the amino acid sequence of a protein set forth in Table 3. In some embodiments, the TL1A binding protein consists of an amino acid sequence 100% identical to the amino acid sequence of a protein set forth in Table 3.

In some embodiments, the TL1A binding protein comprises the amino acid sequence of a protein set forth in Table 3, and further comprising 1 or more but less than 15% (less than 12%, less than 10%, less than 8%), amino acid variations (e.g., substitutions, additions, deletions, etc.). In some embodiments, the TL1A binding protein comprises the amino acid sequence of a protein set forth in Table 3, and further comprising at least 1, 2, 3, 4, 5, 6, 7, 8, 9, or 10 amino acid variations (e.g., substitutions, additions, deletions, etc.). In some embodiments, the TL1A binding protein comprises the amino acid sequence of a protein set forth in Table 3, and further consisting of at least 1, 2, 3, 4, 5, 6, 7, 8, 9, or 10 amino acid variations (e.g., substitutions, additions, deletions, etc.). In some embodiments, the TL1A binding protein comprises the amino acid sequence of a protein set forth in Table 3, and further comprising 1, 2, 3, 4, 5, 6, 7, 8, 9, or 10 amino acid variations (e.g., substitutions, additions, deletions, etc.). In some embodiments, the TL1A binding protein comprises the amino acid sequence of a protein set forth in Table 3, and further consisting of 1, 2, 3, 4, 5, 6, 7, 8, 9, or 10 amino acid variations (e.g., substitutions, additions, deletions, etc.). In some embodiments, the TL1A binding protein comprises the amino acid sequence of a protein set forth in Table 3, and further comprising or no more than 1, 2, 3, 4, 5, 6, 7, 8, 9, or 10 amino acid variations (e.g., substitutions, additions, deletions, etc.).

In some embodiments, the TL1A binding protein consists of the amino acid sequence of a protein set forth in Table 3, and further comprising 1 or more but less than 15% (less than 12%, less than 10%, less than 8%), amino acid variations (e.g., substitutions, additions, deletions, etc.). In some embodiments, the TL1A binding protein consists of the amino acid sequence of a protein set forth in Table 3, and further comprising at least 1, 2, 3, 4, 5, 6, 7, 8, 9, or 10 amino acid variations (e.g., substitutions, additions, deletions, etc.). In some embodiments, the TL1A binding protein consists of the amino acid sequence of a protein set forth in Table 3, and further consisting of at least 1, 2, 3, 4, 5, 6, 7, 8, 9, or 10 amino acid variations (e.g., substitutions, additions, deletions, etc.). In some embodiments, the TL1A binding protein consists of the amino acid sequence of a protein set forth in Table 3, and further comprising 1, 2, 3, 4, 5, 6, 7, 8, 9, or 10 amino acid variations (e.g., substitutions, additions, deletions, etc.). In some embodiments, the TL1A binding protein consists of the amino acid sequence of a protein set forth in Table 3, and further consisting of 1, 2, 3, 4, 5, 6, 7, 8, 9, or 10 amino acid variations (e.g., substitutions, additions, deletions, etc.). In some embodiments, the TL1A binding protein consists of the amino acid sequence of a protein set forth in Table 3, and further comprising or no more than 1, 2, 3, 4, 5, 6, 7, 8, 9, or 10 amino acid variations (e.g., substitutions, additions, deletions, etc.).

In some embodiments, the TL1A binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, the TL1A binding protein comprises an amino acid sequence at least 85% identical to the amino acid sequence set forth in any one of SEQ ID NOS: 387-454. In some embodiments, the TL1A binding protein comprises an amino acid sequence at least 90% identical to the amino acid sequence set forth in any one of SEQ ID NOS: 387-454. In some embodiments, the TL1A binding protein comprises an amino acid sequence at least 95% identical to the amino acid sequence set forth in any one of SEQ ID NOS: 387-454. In some embodiments, the TL1A binding protein comprises an amino acid sequence at least 99% identical to the amino acid sequence set forth in any one of SEQ ID NOS: 387-454.

In some embodiments, the TL1A binding protein comprises an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, the TL1A binding protein comprises an amino acid sequence 85% identical to the amino acid sequence set forth in any one of SEQ ID NOS: 387-454. In some embodiments, the TL1A binding protein comprises an amino acid sequence 90% identical to the amino acid sequence set forth in any one of SEQ ID NOS: 387-454. In some embodiments, the TL1A binding protein comprises an amino acid sequence 95% identical to the amino acid sequence set forth in any one of SEQ ID NOS: 387-454. In some embodiments, the TL1A binding protein comprises an amino acid sequence 99% identical to the amino acid sequence set forth in any one of SEQ ID NOS: 387-454. In some embodiments, the TL1A binding protein comprises an amino acid sequence 100% identical to the amino acid sequence set forth in any one of SEQ ID NOS: 387-454.

In some embodiments, the TL1A binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, the TL1A binding protein consists of an amino acid sequence at least 85% identical to the amino acid sequence set forth in any one of SEQ ID NOS: 387-454. In some embodiments, the TL1A binding protein consists of an amino acid sequence at least 90% identical to the amino acid sequence set forth in any one of SEQ ID NOS: 387-454. In some embodiments, the TL1A binding protein consists of an amino acid sequence at least 95% identical to the amino acid sequence set forth in any one of SEQ ID NOS: 387-454. In some embodiments, the TL1A binding protein consists of an amino acid sequence at least 99% identical to the amino acid sequence set forth in any one of SEQ ID NOS: 387-454.

In some embodiments, the TL1A binding protein comprises the amino acid sequence set forth in any one of SEQ ID NOS: 387-454, and further comprising 1 or more but less than 15% (less than 12%, less than 10%, less than 8%), amino acid variations (e.g., substitutions, additions, deletions, etc.). In some embodiments, the TL1A binding protein comprises the amino acid sequence set forth in any one of SEQ ID NOS: 387-454, and further comprising at least 1, 2, 3, 4, 5, 6, 7, 8, 9, or 10 amino acid variations (e.g., substitutions, additions, deletions, etc.). In some embodiments, the TL1A binding protein comprises the amino acid sequence set forth in any one of SEQ ID NOS: 387-454, and further comprising 1, 2, 3, 4, 5, 6, 7, 8, 9, or 10 amino acid variations (e.g., substitutions, additions, deletions, etc.). In some embodiments, the TL1A binding protein comprises the amino acid sequence set forth in any one of SEQ ID NOS: 387-454, and further consisting of 1, 2, 3, 4, 5, 6, 7, 8, 9, or 10 amino acid variations (e.g., substitutions, additions, deletions, etc.). In some embodiments, the TL1A binding protein comprises the amino acid sequence set forth in any one of SEQ ID NOS: 387-454, and further comprising or consists of no more than 1, 2, 3, 4, 5, 6, 7, 8, 9, or 10 amino acid variations (e.g., substitutions, additions, deletions, etc.).

In some embodiments, the TL1A binding protein consists of the amino acid sequence set forth in any one of SEQ ID NOS: 387-454, and further comprising 1 or more but less than 15% (less than 12%, less than 10%, less than 8%), amino acid variations (e.g., substitutions, additions, deletions, etc.). In some embodiments, the TL1A binding protein consists of the amino acid sequence set forth in any one of SEQ ID NOS: 387-454, and further comprising at least 1, 2, 3, 4, 5, 6, 7, 8, 9, or 10 amino acid variations (e.g., substitutions, additions, deletions, etc.). In some embodiments, the TL1A binding protein consists of the amino acid sequence set forth in any one of SEQ ID NOS: 387-454, and further comprising 1, 2, 3, 4, 5, 6, 7, 8, 9, or 10 amino acid variations (e.g., substitutions, additions, deletions, etc.). In some embodiments, the TL1A binding protein consists of the amino acid sequence set forth in any one of SEQ ID NOS: 387-454, and further consisting of 1, 2, 3, 4, 5, 6, 7, 8, 9, or 10 amino acid variations (e.g., substitutions, additions, deletions, etc.). In some embodiments, the TL1A binding protein consists of the amino acid sequence set forth in any one of SEQ ID NOS: 387-454, and further consisting of no more than 1, 2, 3, 4, 5, 6, 7, 8, 9, or 10 amino acid variations (e.g., substitutions, additions, deletions, etc.).

In some aspects and embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 3. In some embodiments, a fusion protein described herein comprises a protein (or a functional fragment, functional variant, or functional fragment/variant thereof) comprising an amino acid sequence at least 85% identical to the amino acid sequence of a protein set forth in Table 3. In some embodiments, a fusion protein described herein comprises a protein (or a functional fragment, functional variant, or functional fragment/variant thereof) comprising an amino acid sequence at least 90% identical to the amino acid sequence of a protein set forth in Table 3. In some embodiments, a fusion protein described herein comprises a protein (or a functional fragment, functional variant, or functional fragment/variant thereof) comprising an amino acid sequence at least 95% identical to the amino acid sequence of a protein set forth in Table 3. In some embodiments, a fusion protein described herein comprises a protein (or a functional fragment, functional variant, or functional fragment/variant thereof) comprising an amino acid sequence at least 99% identical to the amino acid sequence of a protein set forth in Table 3.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 3. In some embodiments, a fusion protein described herein comprises a protein (or a functional fragment, functional variant, or functional fragment/variant thereof) comprising an amino acid sequence 85% identical to the amino acid sequence of a protein set forth in Table 3. In some embodiments, a fusion protein described herein comprises a protein ((or a functional fragment, functional variant, or functional fragment/variant thereof) comprising an amino acid sequence 90% identical to the amino acid sequence of a protein set forth in Table 3. In some embodiments, a fusion protein described herein comprises a protein (or a functional fragment, functional variant, or functional fragment/variant thereof) comprising an amino acid sequence 95% identical to the amino acid sequence of a protein set forth in Table 3. In some embodiments, a fusion protein described herein comprises a protein (or a functional fragment, functional variant, or functional fragment/variant thereof) comprising an amino acid sequence 99% identical to the amino acid sequence of a protein set forth in Table 3. In some embodiments, a fusion protein described herein comprises a protein (or a functional fragment, functional variant, or functional fragment/variant thereof) comprising an amino acid sequence 100% identical to the amino acid sequence of a protein set forth in Table 3.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 3. In some embodiments, a fusion protein described herein comprises a protein (or a functional fragment, functional variant, or functional fragment/variant thereof) consisting of an amino acid sequence at least 85% identical to the amino acid sequence of a protein set forth in Table 3. In some embodiments, a fusion protein described herein comprises a protein (or a functional fragment, functional variant, or functional fragment/variant thereof) consisting of an amino acid sequence at least 90% identical to the amino acid sequence of a protein set forth in Table 3. In some embodiments, a fusion protein described herein comprises a protein (or a functional fragment, functional variant, or functional fragment/variant thereof) consisting of an amino acid sequence at least 95% identical to the amino acid sequence of a protein set forth in Table 3. In some embodiments, a fusion protein described herein comprises a protein (or a functional fragment, functional variant, or functional fragment/variant thereof) consisting of an amino acid sequence at least 99% identical to the amino acid sequence of a protein set forth in Table 3.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 3. In some embodiments, a fusion protein described herein comprises a protein (or a functional fragment, functional variant, or functional fragment/variant thereof) consisting of an amino acid sequence 85% identical to the amino acid sequence of a protein set forth in Table 3. In some embodiments, a fusion protein described herein comprises a protein (or a functional fragment, functional variant, or functional fragment/variant thereof) consisting of an amino acid sequence 90% identical to the amino acid sequence of a protein set forth in Table 3. In some embodiments, a fusion protein described herein comprises a protein (or a functional fragment, functional variant, or functional fragment/variant thereof) consisting of an amino acid sequence 95% identical to the amino acid sequence of a protein set forth in Table 3. In some embodiments, a fusion protein described herein comprises a protein (or a functional fragment, functional variant, or functional fragment/variant thereof) consisting of an amino acid sequence 99% identical to the amino acid sequence of a protein set forth in Table 3. In some embodiments, a fusion protein described herein comprises a protein (or a functional fragment, functional variant, or functional fragment/variant thereof) consisting of an amino acid sequence 100% identical to the amino acid sequence of a protein set forth in Table 3.

In some embodiments, a fusion protein described herein comprises a protein (or a functional fragment, functional variant, or functional fragment/variant thereof) comprising the amino acid sequence of a protein set forth in Table 3, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 3, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 3, and further consisting of at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 3, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 3, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 3, and further comprising or no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence of a protein set forth in Table 3, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 3, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 3, and further consisting of at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 3, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 3, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 3, and further comprising or no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence at least 85% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence at least 90% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence at least 95% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence at least 99% identical to the amino acid sequence set forth in any one of SEQ ID NOS: 387-454.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence 85% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence 90% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence 95% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence 99% identical to the amino acid sequence set forth in any one of SEQ ID NOS: 387-454. In some embodiments, a fusion protein described herein comprises a protein (or a functional fragment, functional variant, or functional fragment/variant thereof) comprising an amino acid sequence 100% identical to the amino acid sequence set forth in any one of SEQ ID NOS: 387-454.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence at least 85% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence at least 90% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence at least 95% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence at least 99% identical to the amino acid sequence set forth in any one of SEQ ID NOS: 387-454.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence 85% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence 90% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence 95% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence 99% identical to the amino acid sequence set forth in any one of SEQ ID NOS: 387-454. In some embodiments, a fusion protein described herein comprises a protein (or a functional fragment, functional variant, or functional fragment/variant thereof) consisting of an amino acid sequence 100% identical to the amino acid sequence set forth in any one of SEQ ID NOS: 387-454.

In some embodiments, a fusion protein described herein comprises a protein (or a functional fragment, functional variant, or functional fragment/variant thereof) comprising the amino acid sequence set forth in any one of SEQ ID NOS: 387-454, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 387-454, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 387-454, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 387-454, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 387-454, and further comprising or consists of no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence set forth in any one of SEQ ID NOS: 387-454, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 387-454, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 387-454, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 387-454, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 387-454, and further consisting of no more than 1, 2, 3, 4, 5, 6, 7, 8, 9, or 10 amino acid variations (e.g., substitutions, additions, deletions, etc.).

### (i) Exemplary Properties of TL1A Binding Proteins

In some embodiments, the TL1A binding proteins described herein are immunosuppressive (e.g., when administered to a subject). In some embodiments, the TL1A binding proteins described herein are anti-inflammatory (e.g., when administered to a subject). In some embodiments, the TL1A binding proteins described herein suppress pro-inflammatory response (e.g., when administered to a subject). In some embodiments, the TL1A binding protein can act as a decoy receptor for a TNFSF ligand described herein.

In some embodiments, the TL1A binding protein is capable of specifically binding hTL1A. In some embodiments, the TL1A binding protein is capable of inhibiting or reducing (e.g., preventing) binding of hTL1A to hDR3.

In some embodiments, the TL1A binding proteins described herein bind a subset (e.g., one or more) TNFSF ligand. In some embodiments, the TL1A binding protein specifically binds TL1A. In some embodiments, the TL1A binding protein specifically binds hTL1A.

In some embodiments, the TL1A binding protein inhibits reduces (e.g., prevents) binding of TL1A to DR3. In some embodiments, the TL1A binding protein specifically binds to TL1A and inhibits reduces (e.g., prevents) binding of TL1A to DR3. In some embodiments, the TL1A binding protein inhibits reduces (e.g., prevents) binding of hTL1A to hDR3. In some embodiments, the TL1A binding protein specifically binds to hTL1A and inhibits reduces (e.g., prevents) binding of hTL1A to hDR3. In some embodiments, the TL1A binding protein inhibits reduces (e.g., prevents) DR3 signaling. In some embodiments, the TL1A binding protein inhibits reduces (e.g., prevents) DR3 signaling mediated by DR3 binding to TL1A. In some embodiments, the TL1A binding protein inhibits reduces (e.g., prevents) NFκB activation. In some embodiments, the TL1A binding protein inhibits reduces (e.g., prevents) NFκB activation mediating through DR3. In some embodiments, the TL1A binding protein inhibits reduces (e.g., prevents) NFκB activation mediated through DR3 binding to TL1A.

In some embodiments, the TL1A binding protein inhibits reduces (e.g., prevents) binding of TL1A to DcR3. In some embodiments, the TL1A binding protein specifically binds to TL1A and inhibits reduces (e.g., prevents) binding of TL1A to DcR3. In some embodiments, the TL1A binding protein inhibits reduces (e.g., prevents) binding of hTL1A to hDcR3. In some embodiments, the TL1A binding protein specifically binds to hTL1A and inhibits reduces (e.g., prevents) binding of hTL1A to hDcR3. In some embodiments, the TL1A binding protein inhibits reduces (e.g., prevents) DcR3 signaling. In some embodiments, the TL1A binding protein inhibits reduces (e.g., prevents) DcR3 signaling mediated by DcR3 binding to TL1A.

In some embodiments, the TL1A binding protein does not specifically binds one or more of TNFα, LIGHT, and/or FASL. In some embodiments, the TL1A binding protein does not specifically bind 2 or 3 of TNFα, LIGHT, and/or FASL. In some embodiments, the TL1A binding protein does not specifically bind TNFα, LIGHT, and/or FASL. In some embodiments, the TL1A binding protein does not specifically bind TNFα, LIGHT, and/or FASL. In some embodiments, the TL1A binding protein does not specifically bind a plurality of TNFα, LIGHT, and/or FASL.

In some embodiments, the TL1A binding protein does not specifically binds one or more of hTNFα, hLIGHT, and/or hFASL. In some embodiments, the TL1A binding protein does not specifically bind 2 or 3 of hTNFα, hLIGHT, and/or hFASL. In some embodiments, the TL1A binding protein does not specifically bind hTNFα, hLIGHT, and/or hFASL. In some embodiments, the TL1A binding protein does not specifically bind hTNFα, hLIGHT, and/or hFASL. In some embodiments, the TL1A binding protein does not specifically bind a plurality of hTNFα, hLIGHT, and/or hFASL.

In some embodiments, the TL1A binding protein does not inhibit or reduce (e.g., prevent) binding of one or more TNFSF ligand (e.g., TNFα, LIGHT, and/or FASL) to one or more of the TNFSF ligand's (e.g., TNFα, LIGHT, and/or FASL) cognate receptor.

In some embodiments, the TL1A binding protein exhibits one or more (e.g., 1, 2, 3, 4, and/or 5) of (a) does not inhibit or reduce (e.g., prevent)binding of TNFα to TNFR1, (b) does not inhibit or reduce (e.g., prevent)binding of TNFα to TNFR2, (c) does not inhibit or reduce (e.g., prevent)binding of LIGHT to LIGHTR, (d) does not inhibit or reduce (e.g., prevent)binding of LIGHT to LTβR, and/or (e) does not inhibit or reduce (e.g., prevent)binding of FASL to FAS. In some embodiments, the TL1A binding protein does not inhibit or reduce (e.g., prevent) binding of TNFα to TNFR1. In some embodiments, the TL1A binding protein does not inhibit or reduce (e.g., prevent) binding of TNFα to TNFR2. In some embodiments, the TL1A binding protein does not inhibit or reduce (e.g., prevent) binding of LIGHT to LIGHTR. In some embodiments, the TL1A binding protein does not inhibit or reduce (e.g., prevent) binding of LIGHT to LTβR. In some embodiments, the TL1A binding protein does not inhibit or reduce (e.g., prevent) binding of FASL to FAS. In some embodiments, the TL1A binding protein (a) does not inhibit or reduce (e.g., prevent) binding of TNFα to TNFR1, (b) does not inhibit or reduce (e.g., prevent) binding of TNFα to TNFR2, (c) does not inhibit or reduce (e.g., prevent) binding of LIGHT to LIGHTR, (d) does not inhibit or reduce (e.g., prevent) binding of LIGHT to LTβR, and (e) does not inhibit or reduce (e.g., prevent) binding of FASL to FAS.

In some embodiments, the TL1A binding protein exhibits one or more (e.g., 1, 2, 3, 4, and/or 5) of (a) does not specifically bind to TNFα and does not inhibit or reduce (e.g., prevent) binding of TNFα to TNFR1, (b) does not specifically bind to TNFα and does not inhibit or reduce (e.g., prevent) binding of TNFα to TNFR2, (c) does not specifically bind to LIGHT and does not inhibit or reduce (e.g., prevent) binding of LIGHT to LIGHTR, (d) does not specifically bind to LIGHT and does not inhibit or reduce (e.g., prevent) binding of LIGHT to LTβR, and/or (e) does not specifically bind to FASL and does not inhibit or reduce (e.g., prevent) binding of FASL to FAS. In some embodiments, the TL1A binding protein does not specifically bind to TNFα and does not inhibit or reduce (e.g., prevent) binding of TNFα to TNFR1. In some embodiments, the TL1A binding protein does not specifically bind to TNFα and does not inhibit or reduce (e.g., prevent) binding of TNFα to TNFR2. In some embodiments, the TL1A binding protein does not specifically bind to LIGHT and does not inhibit or reduce (e.g., prevent) binding of LIGHT to LIGHTR. In some embodiments, the TL1A binding protein does not specifically bind to LIGHT and does not inhibit or reduce (e.g., prevent) binding of LIGHT to LTβR. In some embodiments, the TL1A binding protein does not specifically bind to FASL and does not inhibit or reduce (e.g., prevent) binding of FASL to FAS. In some embodiments, the TL1A binding protein (a) does not specifically bind to TNFα and does not inhibit or reduce (e.g., prevent) binding of TNFα to TNFR1, (b) does not specifically bind to TNFα and does not inhibit or reduce (e.g., prevent) binding of TNFα to TNFR2, (c) does not specifically bind to LIGHT and does not inhibit or reduce (e.g., prevent) binding of LIGHT to LIGHTR, (d) does not specifically bind to LIGHT and does not inhibit or reduce (e.g., prevent) binding of LIGHT to LTβR, and (e) does not specifically bind to FASL and does not inhibit or reduce (e.g., prevent) binding of FASL to FAS.

In some embodiments, the TL1A binding protein exhibits one or more (e.g., 1, 2, 3, 4, and/or 5) of (a) does not inhibit or reduce (e.g., prevent) binding of hTNFα to hTNFR1, (b) does not inhibit or reduce (e.g., prevent) binding of hTNFα to hTNFR2, (c) does not inhibit or reduce (e.g., prevent) binding of hLIGHT to hLIGHTR, (d) does not inhibit or reduce (e.g., prevent) binding of hLIGHT to hLTβR, and/or (e) does not inhibit or reduce (e.g., prevent) binding of hFASL to hFAS. In some embodiments, the TL1A binding protein does not inhibit or reduce (e.g., prevent) binding of hTNFα to hTNFR1. In some embodiments, the TL1A binding protein does not inhibit or reduce (e.g., prevent) binding of hTNFα to hTNFR2. In some embodiments, the TL1A binding protein does not inhibit or reduce (e.g., prevent) binding of hLIGHT to hLIGHTR. In some embodiments, the TL1A binding protein does not inhibit or reduce (e.g., prevent) binding of hLIGHT to hLTβR. In some embodiments, the TL1A binding protein does not inhibit or reduce (e.g., prevent) binding of FASL to FAS. In some embodiments, the TL1A binding protein (a) does not inhibit or reduce (e.g., prevent) binding of hTNFα to hTNFR1, (b) does not inhibit or reduce (e.g., prevent) binding of hTNFα to hTNFR2, (c) does not inhibit or reduce (e.g., prevent) binding of hLIGHT to hLIGHTR, (d) does not inhibit or reduce (e.g., prevent) binding of hLIGHT to hLTβR, and (e) does not inhibit or reduce (e.g., prevent) binding of hFASL to hFAS.

In some embodiments, the TL1A binding protein exhibits one or more (e.g., 1, 2, 3, 4, and/or 5) of (a) does not specifically bind to hTNFα and does not inhibit or reduce (e.g., prevent) binding of hTNFα to hTNFR1, (b) does not specifically bind to hTNFα and does not inhibit or reduce (e.g., prevent) binding of hTNFα to hTNFR2, (c) does not specifically bind to hLIGHT and does not inhibit or reduce (e.g., prevent) binding of hLIGHT to hLIGHTR, (d) does not specifically bind to hLIGHT and does not inhibit or reduce (e.g., prevent) binding of hLIGHT to hLTβR, and/or (e) does not specifically bind to hFASL and does not inhibit or reduce (e.g., prevent) binding of hFASL to hFAS. In some embodiments, the TL1A binding protein does not specifically bind to hTNFα and does not inhibit or reduce (e.g., prevent) binding of hTNFα to hTNFR1. In some embodiments, the TL1A binding protein does not specifically bind to hTNFα and does not inhibit or reduce (e.g., prevent) binding of hTNFα to hTNFR2. In some embodiments, the TL1A binding protein does not specifically bind to hLIGHT and does not inhibit or reduce (e.g., prevent) binding of hLIGHT to hLIGHTR. In some embodiments, the TL1A binding protein does not specifically bind to hLIGHT and does not inhibit or reduce (e.g., prevent) binding of hLIGHT to hLTβR. In some embodiments, the TL1A binding protein does not specifically bind to hFASL and does not inhibit or reduce (e.g., prevent) binding of hFASL to hFAS. In some embodiments, the TL1A binding protein (a) does not specifically bind to hTNFα and does not inhibit or reduce (e.g., prevent) binding of hTNFα to hTNFR1, (b) does not specifically bind to hTNFα and does not inhibit or reduce (e.g., prevent) binding of hTNFα to hTNFR2, (c) does not specifically bind to hLIGHT and does not inhibit or reduce (e.g., prevent) binding of hLIGHT to hLIGHTR, (d) does not specifically bind to hLIGHT and does not inhibit or reduce (e.g., prevent) binding of hLIGHT to hLTβR, and (e) does not specifically bind to hFASL and does not inhibit or reduce (e.g., prevent) binding of hFASL to hFAS.

In some embodiments, the TL1A binding protein specifically binds one or more of TNFα, LIGHT, FASL, and/or TL1A. In some embodiments, the TL1A binding protein specifically binds 2, 3, or 4 of TNFα, LIGHT, FASL, and/or TL1A. In some embodiments, the TL1A binding protein specifically binds TNFα, LIGHT, FASL, and/or TL1A. In some embodiments, the TL1A binding protein specifically binds TNFα, LIGHT, FASL, and TL1A. In some embodiments, the TL1A binding protein specifically binds a plurality of TNFα, LIGHT, FASL, and/or TL1A.

In some embodiments, the TL1A binding protein specifically binds one or more of hTNFα, hLIGHT, hFASL, and/or hTL1A. In some embodiments, the TL1A binding protein specifically binds 2, 3, or 4 of hTNFα, hLIGHT, hFASL, and/or hTL1A. In some embodiments, the TL1A binding protein specifically binds hTNFα, hLIGHT, hFASL, and/or hTL1A. In some embodiments, the TL1A binding protein specifically binds hTNFα, hLIGHT, hFASL, and hTL1A. In some embodiments, the TL1A binding protein specifically binds a plurality of hTNFα, hLIGHT, hFASL, and/or hTL1A.

In some embodiments, the TL1A binding protein inhibits or reduces (e.g., prevents) binding of one or more TNFSF ligand (e.g., TNFα, LIGHT, FASL, and/or TL1A) to one or more of the TNFSF ligand's (e.g., TNFα, LIGHT, FASL, and/or TL1A) cognate receptor.

In some embodiments, the TL1A binding protein exhibits one or more (e.g., 1, 2, 3, 4, 5, and/or 6) of (a) inhibits or reduces (e.g., prevents) binding of TNFα to TNFR1, (b) inhibits or reduces (e.g., prevents) binding of TNFα to TNFR2, (c) inhibits or reduces (e.g., prevents) binding of LIGHT to LIGHTR, (d) inhibits or reduces (e.g., prevents) binding of LIGHT to LTβR, (e) inhibits or reduces (e.g., prevents) binding of FASL to FAS, and/or (f) inhibits or reduces (e.g., prevents) binding of TL1A to DR3. In some embodiments, the TL1A binding protein inhibits or reduces (e.g., prevents) binding of TNFα to TNFR1. In some embodiments, the TL1A binding protein inhibits or reduces (e.g., prevents) binding of TNFα to TNFR2. In some embodiments, the TL1A binding protein inhibits or reduces (e.g., prevents) binding of LIGHT to LIGHTR. In some embodiments, the TL1A binding protein inhibits or reduces (e.g., prevents) binding of LIGHT to LTβR. In some embodiments, the TL1A binding protein inhibits or reduces (e.g., prevents) binding of FASL to FAS. In some embodiments, the TL1A binding protein inhibits or reduces (e.g., prevents) binding of TL1A to DR3. In some embodiments, the TL1A binding protein (a) inhibits or reduces (e.g., prevents) binding of TNFα to TNFR1, (b) inhibits or reduces (e.g., prevents) binding of TNFα to TNFR2, (c) inhibits or reduces (e.g., prevents) binding of LIGHT to LIGHTR, (d) inhibits or reduces (e.g., prevents) binding of LIGHT to LTβR, (e) inhibits or reduces (e.g., prevents) binding of FASL to FAS, and/or (f) inhibits or reduces (e.g., prevents) binding of TL1A to DR3.

In some embodiments, the TL1A binding protein exhibits one or more (e.g., 1, 2, 3, 4, 5, and/or 6) of (a) specifically binds to TNFα and inhibits or reduces (e.g., prevents) binding of TNFα to TNFR1, (b) specifically binds to TNFα and inhibits or reduces (e.g., prevents) binding of TNFα to TNFR2, (c) specifically binds to LIGHT and inhibits or reduces (e.g., prevents) binding of LIGHT to LIGHTR, (d) specifically binds to LIGHT and inhibits or reduces (e.g., prevents) binding of LIGHT to LTβR, (e) specifically binds to FASL and inhibits or reduces (e.g., prevents) binding of FASL to FAS, and/or (f) specifically binds to TL1A and inhibits or reduces (e.g., prevents) binding of TL1A to DR3. In some embodiments, the TL1A binding protein specifically binds to TNFα and inhibits or reduces (e.g., prevents) binding of TNFα to TNFR1. In some embodiments, the TL1A binding protein specifically binds to TNFα and inhibits or reduces (e.g., prevents) binding of TNFα to TNFR2. In some embodiments, the TL1A binding protein specifically binds to LIGHT and inhibits or reduces (e.g., prevents) binding of LIGHT to LIGHTR. In some embodiments, the TL1A binding protein specifically binds to LIGHT and inhibits or reduces (e.g., prevents) binding of LIGHT to LTβR. In some embodiments, the TL1A binding protein specifically binds to FASL and inhibits or reduces (e.g., prevents) binding of FASL to FAS. In some embodiments, the TL1A binding protein specifically binds to TL1A and inhibits or reduces (e.g., prevents) binding of TL1A to DR3. In some embodiments, the TL1A binding protein (a) specifically binds to TNFα and inhibits or reduces (e.g., prevents) binding of TNFα to TNFR1, (b) specifically binds to TNFα and inhibits or reduces (e.g., prevents) binding of TNFα to TNFR2, (c) specifically binds to LIGHT and inhibits or reduces (e.g., prevents) binding of LIGHT to LIGHTR, (d) specifically binds to LIGHT and inhibits or reduces (e.g., prevents) binding of LIGHT to LTβR, (e) specifically binds to FASL and inhibits or reduces (e.g., prevents) binding of FASL to FAS, and (f) specifically binds to TL1A and inhibits or reduces (e.g., prevents) binding of TL1A to DR3.

In some embodiments, the TL1A binding protein exhibits one or more (e.g., 1, 2, 3, 4, 5, and/or 6) of (a) inhibits or reduces (e.g., prevents) binding of hTNFα to hTNFR1, (b) inhibits or reduces (e.g., prevents) binding of hTNFα to hTNFR2, (c) inhibits or reduces (e.g., prevents) binding of hLIGHT to hLIGHTR, (d) inhibits or reduces (e.g., prevents) binding of hLIGHT to hLTβR, (e) inhibits or reduces (e.g., prevents) binding of hFASL to hFAS, and/or (f) inhibits or reduces (e.g., prevents) binding of hTL1A to hDR3. In some embodiments, the TL1A binding protein inhibits or reduces (e.g., prevents) binding of hTNFα to hTNFR1. In some embodiments, the TL1A binding protein inhibits or reduces (e.g., prevents) binding of hTNFα to hTNFR2. In some embodiments, the TL1A binding protein inhibits or reduces (e.g., prevents) binding of hLIGHT to hLIGHTR. In some embodiments, the TL1A binding protein inhibits or reduces (e.g., prevents) binding of hLIGHT to hLTβR. In some embodiments, the TL1A binding protein inhibits or reduces (e.g., prevents) binding of FASL to FAS. In some embodiments, the TL1A binding protein inhibits or reduces (e.g., prevents) binding of hTL1A to hDR3. In some embodiments, the TL1A binding protein (a) inhibits or reduces (e.g., prevents) binding of hTNFα to hTNFR1, (b) inhibits or reduces (e.g., prevents) binding of hTNFα to hTNFR2, (c) inhibits or reduces (e.g., prevents) binding of hLIGHT to hLIGHTR, (d) inhibits or reduces (e.g., prevents) binding of hLIGHT to hLTβR, (e) inhibits or reduces (e.g., prevents) binding of hFASL to hFAS, and (f) inhibits or reduces (e.g., prevents) binding of hTL1A to hDR3.

In some embodiments, the TL1A binding protein exhibits one or more (e.g., 1, 2, 3, 4, 5, and/or 6) of (a) specifically binds to hTNFα and inhibits or reduces (e.g., prevents) binding of hTNFα to hTNFR1, (b) specifically binds to hTNFα and inhibits or reduces (e.g., prevents) binding of hTNFα to hTNFR2, (c) specifically binds to hLIGHT and inhibits or reduces (e.g., prevents) binding of hLIGHT to hLIGHTR, (d) specifically binds to hLIGHT and inhibits or reduces (e.g., prevents) binding of hLIGHT to hLTβR, (e) specifically binds to hFASL and inhibits or reduces (e.g., prevents) binding of hFASL to hFAS, and/or (f) specifically binds to hTL1A and inhibits or reduces (e.g., prevents) binding of hTL1A to hDR3. In some embodiments, the TL1A binding protein specifically binds to hTNFα and inhibits or reduces (e.g., prevents) binding of hTNFα to hTNFR1. In some embodiments, the TL1A binding protein specifically binds to hTNFα and inhibits or reduces (e.g., prevents) binding of hTNFα to hTNFR2. In some embodiments, the TL1A binding protein specifically binds to hLIGHT and inhibits or reduces (e.g., prevents) binding of hLIGHT to hLIGHTR. In some embodiments, the TL1A binding protein specifically binds to hLIGHT and inhibits or reduces (e.g., prevents) binding of hLIGHT to hLTβR. In some embodiments, the TL1A binding protein specifically binds to hFASL and inhibits or reduces (e.g., prevents) binding of hFASL to hFAS. In some embodiments, the TL1A binding protein specifically binds to hTL1A and inhibits or reduces (e.g., prevents) binding of hTL1A to hDR3. In some embodiments, the TL1A binding protein (a) specifically binds to hTNFα and inhibits or reduces (e.g., prevents) binding of hTNFα to hTNFR1, (b) specifically binds to hTNFα and inhibits or reduces (e.g., prevents) binding of hTNFα to hTNFR2, (c) specifically binds to hLIGHT and inhibits or reduces (e.g., prevents) binding of hLIGHT to hLIGHTR, (d) specifically binds to hLIGHT and inhibits or reduces (e.g., prevents) binding of hLIGHT to hLTβR, (e) specifically binds to hFASL and inhibits or reduces (e.g., prevents) binding of hFASL to hFAS, and (f) specifically binds to hTL1A and inhibits or reduces (e.g., prevents) binding of hTL1A to hDR3.

For the sake of clarity, and as further described herein, where an immunoreceptor inhibitory protein described herein specifically binds more than one TNFSF ligand (e.g., specifically binds TNFα, LIGHT, FASL, and TL1A), the binding affinity of the TL1A binding protein for each TNFSF ligand can vary.

In some embodiments, the TL1A binding protein specifically binds heparan sulfate proteoglycans. In some embodiments, the TL1A binding protein comprises a heparan sulfate proteoglycan binding domain. In some embodiments, the TL1A binding protein does not specifically bind heparan sulfate proteoglycans. In some embodiments, the TL1A binding protein does not comprise a heparan sulfate proteoglycan binding domain.

### 5.2.1.3 TNFα Binding Proteins

In some aspects and embodiments, a fusion protein described herein (or a combination regimen described herein) comprises a protein that specifically binds the hTNFα.

The amino acid sequence of proteins that are capable of specifically binding hTNFα is set forth in Table 4 (SEQ ID NOS: 455-464). The amino acid sequence of the mature form of the proteins (i.e., lacking the native signal peptide) is set forth in SEQ ID NOS: 455-459. The amino acid sequence of the immature form of the proteins (i.e., containing the native signal peptide) is set forth in SEQ ID NOS: 460-464.

The signal peptides have been computationally predicted using standard methods (see, e.g., Teufel 2022). A person of ordinary skill in the art would know how to experimentally identify and/or validate a computationally predicted signal peptide using standard methods known in the art, e.g., expression of a protein from a host cell and sequencing of the intracellular form and the extracellular form of the expressed protein (see, e.g., Zhang 2004).

TABLE 4
The Amino Acid Sequence of TNFα Binding Proteins.

| | | SEQ ID |
|---|---|---|
| Description | Amino Acid Sequence | NO |
| IMP-210 | DSKCGVSEYYNKEHDICCRLCPAGSYAEQLCTKDNDTVCNQCPPNT | 455 |
| without native signal | FLSIPNYISSCLSCRGKCINDHVEDKPCTATSNRICKCKENKTCVL | |
| peptide | KTYDNSCRVCI | |
| IMP-211 | SLQCKNNTYYNSQYVKCCKLCEPGTFYSKKCDEKNDTICEKCPDGS | 456 |
| without native signal | YTSVYNHSPACVSCRGYCDYNQVETTSCTPTSNRICKCKLSSYCLV | |
| peptide | KGYNENCRVCVRKKMN | |
| IMP-212 | DIAPHAPSDGKCKDNEYKRHNLCPGTYASRLCDSKTNTQCTPCGSG | 457 |
| without native signal | TFTSRNNHLPACLSCNGRRDRVTRLTIESVNALPDIIVFSKDHPDA | |
| peptide | RHVFPKQNVE | |
| IMP-213 | DIAPHAPSDGKCKDNEYKRHNLCPGTYASDSKTNTRCTPCGSGTFT | 458 |
| without native signal | SRNNHLPACLSCNGRRDRVTRLTIESVNALPDIIVFSKDHPDARHV | |
| peptide | FPKQNVE | |
| IMP-214 | DNNCGELEYYNKVHDVCCKLCPAGFYAKQLCTKDMDTVCNPCATET | 459 |
| without native signal | FLSIPNYTSKCLSCRGKCTKDQVEVRPCTITRNRTCKCKDGYICIL | |
| peptide | KTDDNSCRVCV | |
| IMP-210 | MYKYSNYILYLLILVTVARSSDSKCGVSEYYNKEHDICCRLCPAGS | 460 |
| with native signal | YAEQLCTKDNDTVCNQCPPNTFLSIPNYISSCLSCRGKCINDHVED | |
| peptide | KPCTATSNRICKCKENKTCVLKTYDNSCRVCI | |
| IMP-211 | MVKLIVFIIGLLINSTYSLSLQCKNNTYYNSQYVKCCKLCEPGTFY | 461 |
| with native signal | SKKCDEKNDTICEKCPDGSYTSVYNHSPACVSCRGYCDYNQVETTS | |
| peptide | CTPTSNRICKCKLSSYCLVKGYNENCRVCVRKKMN | |
| IMP-212 | MKSVLYSYILFLSCIIINGRDIAPHAPSDGKCKDNEYKRHNLCPGT | 462 |
| with native signal | YASRLCDSKTNTQCTPCGSGTFTSRNNHLPACLSCNGRRDRVTRLT | |
| peptide | IESVNALPDIIVFSKDHPDARHVFPKQNVE | |
| IMP-213 | MKSVLYSYILFLSCIIINGRDIAPHAPSDGKCKDNEYKRHNLCPGT | 463 |
| with native signal | YASDSKTNTRCTPCGSGTFTSRNNHLPACLSCNGRRDRVTRLTIES | |
| peptide | VNALPDIIVFSKDHPDARHVFPKQNVE | |
| IMP-214 | MYKHCYYILYLFISLTVVCSSDNNCGELEYYNKVHDVCCKLCPAGF | 464 |
| with native signal | YAKQLCTKDMDTVCNPCATETFLSIPNYTSKCLSCRGKCTKDQVEV | |
| peptide | RPCTITRNRTCKCKDGYICILKTDDNSCRVCV | |

In some aspects and embodiments, the TNFα binding protein comprises an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 4. In some embodiments, the TNFα binding protein comprises an amino acid sequence at least about 85% identical to the amino acid sequence of a protein set forth in Table 4. In some embodiments, the TNFα binding protein comprises an amino acid sequence at least about 90% identical to the amino acid sequence of a protein set forth in Table 4. In some embodiments, the TNFα binding protein comprises an amino acid sequence at least about 95% identical to the amino acid sequence of a protein set forth in Table 4. In some embodiments, the TNFα binding protein comprises an amino acid sequence at least about 99% identical to the amino acid sequence of a protein set forth in Table 4.

In some embodiments, the TNFα binding protein comprises an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 4. In some embodiments, the TNFα binding protein comprises an amino acid sequence about 85% identical to the amino acid sequence of a protein set forth in Table 4. In some embodiments, the TNFα binding protein comprises an amino acid sequence about 90% identical to the amino acid sequence of a protein set forth in Table 4. In some embodiments, the TNFα binding protein comprises an amino acid sequence about 95% identical to the amino acid sequence of a protein set forth in Table 4. In some embodiments, the TNFα binding protein comprises an amino acid sequence about 99% identical to the amino acid sequence of a protein set forth in Table 4. In some embodiments, the TNFα binding protein comprises an amino acid sequence about 100% identical to the amino acid sequence of a protein set forth in Table 4.

In some embodiments, the TNFα binding protein consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 4. In some embodiments, the TNFα binding protein consists of an amino acid sequence at least about 85% identical to the amino acid sequence of a protein set forth in Table 4. In some embodiments, the TNFα binding protein consists of an amino acid sequence at least about 90% identical to the amino acid sequence of a protein set forth in Table 4. In some embodiments, the TNFα binding protein consists of an amino acid sequence at least about 95% identical to the amino acid sequence of a protein set forth in Table 4. In some embodiments, the TNFα binding protein consists of an amino acid sequence at least about 99% identical to the amino acid sequence of a protein set forth in Table 4.

In some embodiments, the TNFα binding protein consists of an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 4. In some embodiments, the TNFα binding protein consists of an amino acid sequence about 85% identical to the amino acid sequence of a protein set forth in Table 4. In some embodiments, the TNFα binding protein consists of an amino acid sequence about 90% identical to the amino acid sequence of a protein set forth in Table 4. In some embodiments, the TNFα binding protein consists of an amino acid sequence about 95% identical to the amino acid sequence of a protein set forth in Table 4. In some embodiments, the TNFα binding protein consists of an amino acid sequence about 99% identical to the amino acid sequence of a protein set forth in Table 4. In some embodiments, the TNFα binding protein consists of an amino acid sequence about 100% identical to the amino acid sequence of a protein set forth in Table 4.

In some embodiments, the TNFα binding protein comprises the amino acid sequence of a protein set forth in Table 4, and further comprising 1 or more but less than 15% (less than 12%, less than 10%, less than 8%), amino acid variations (e.g., substitutions, additions, deletions, etc.). In some embodiments, the TNFα binding protein comprises the amino acid sequence of a protein set forth in Table 4, and further comprising at least about 1, 2, 3, 4, 5, 6, 7, 8, 9, or 10 amino acid variations (e.g., substitutions, additions, deletions, etc.). In some embodiments, the TNFα binding protein comprises the amino acid sequence of a protein set forth in Table 4, and further consisting of at least about 1, 2, 3, 4, 5, 6, 7, 8, 9, or 10 amino acid variations (e.g., substitutions, additions, deletions, etc.). In some embodiments, the TNFα binding protein comprises the amino acid sequence of a protein set forth in Table 4, and further comprising about 1, 2, 3, 4, 5, 6, 7, 8, 9, or 10 amino acid variations (e.g., substitutions, additions, deletions, etc.). In some embodiments, the TNFα binding protein comprises the amino acid sequence of a protein set forth in Table 4, and further consisting of about 1, 2, 3, 4, 5, 6, 7, 8, 9, or 10 amino acid variations (e.g., substitutions, additions, deletions, etc.). In some embodiments, the TNFα binding protein comprises the amino acid sequence of a protein set forth in Table 4, and further comprising or no more than about 1, 2, 3, 4, 5, 6, 7, 8, 9, or 10 amino acid variations (e.g., substitutions, additions, deletions, etc.).

In some embodiments, the TNFα binding protein consists of the amino acid sequence of a protein set forth in Table 4, and further comprising 1 or more but less than 15% (less than 12%, less than 10%, less than 8%), amino acid variations (e.g., substitutions, additions, deletions, etc.). In some embodiments, the TNFα binding protein consists of the amino acid sequence of a protein set forth in Table 4, and further comprising at least about 1, 2, 3, 4, 5, 6, 7, 8, 9, or 10 amino acid variations (e.g., substitutions, additions, deletions, etc.). In some embodiments, the TNFα binding protein consists of the amino acid sequence of a protein set forth in Table 4, and further consisting of at least about 1, 2, 3, 4, 5, 6, 7, 8, 9, or 10 amino acid variations (e.g., substitutions, additions, deletions, etc.). In some embodiments, the TNFα binding protein consists of the amino acid sequence of a protein set forth in Table 4, and further comprising about 1, 2, 3, 4, 5, 6, 7, 8, 9, or 10 amino acid variations (e.g., substitutions, additions, deletions, etc.). In some embodiments, the TNFα binding protein consists of the amino acid sequence of a protein set forth in Table 4, and further consisting of about 1, 2, 3, 4, 5, 6, 7, 8, 9, or 10 amino acid variations (e.g., substitutions, additions, deletions, etc.). In some embodiments, the TNFα binding protein consists of the amino acid sequence of a protein set forth in Table 4, and further comprising or no more than about 1, 2, 3, 4, 5, 6, 7, 8, 9, or 10 amino acid variations (e.g., substitutions, additions, deletions, etc.).

In some embodiments, the TNFα binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, the TNFα binding protein comprises an amino acid sequence at least 85% identical to the amino acid sequence set forth in any one of SEQ ID NOS: 455-464. In some embodiments, the TNFα binding protein comprises an amino acid sequence at least 90% identical to the amino acid sequence set forth in any one of SEQ ID NOS: 455-464. In some embodiments, the TNFα binding protein comprises an amino acid sequence at least 95% identical to the amino acid sequence set forth in any one of SEQ ID NOS: 455-464. In some embodiments, the TNFα binding protein comprises an amino acid sequence at least 99% identical to the amino acid sequence set forth in any one of SEQ ID NOS: 455-464.

In some embodiments, the TNFα binding protein comprises an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, the TNFα binding protein comprises an amino acid sequence 85% identical to the amino acid sequence set forth in any one of SEQ ID NOS: 455-464. In some embodiments, the TNFα binding protein comprises an amino acid sequence 90% identical to the amino acid sequence set forth in any one of SEQ ID NOS: 455-464. In some embodiments, the TNFα binding protein comprises an amino acid sequence 95% identical to the amino acid sequence set forth in any one of SEQ ID NOS: 455-464. In some embodiments, the TNFα binding protein comprises an amino acid sequence 99% identical to the amino acid sequence set forth in any one of SEQ ID NOS: 455-464. In some embodiments, the TNFα binding protein comprises an amino acid sequence 100% identical to the amino acid sequence set forth in any one of SEQ ID NOS: 455-464.

In some embodiments, the TNFα binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, the TNFα binding protein consists of an amino acid sequence at least 85% identical to the amino acid sequence set forth in any one of SEQ ID NOS: 455-464. In some embodiments, the TNFα binding protein consists of an amino acid sequence at least 90% identical to the amino acid sequence set forth in any one of SEQ ID NOS: 455-464. In some embodiments, the TNFα binding protein consists of an amino acid sequence at least 95% identical to the amino acid sequence set forth in any one of SEQ ID NOS: 455-464. In some embodiments, the TNFα binding protein consists of an amino acid sequence at least 99% identical to the amino acid sequence set forth in any one of SEQ ID NOS: 455-464.

In some embodiments, the TNFα binding protein consists of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, the TNFα binding protein consists of an amino acid sequence 85% identical to the amino acid sequence set forth in any one of SEQ ID NOS: 455-464. In some embodiments, the TNFα binding protein consists of an amino acid sequence 90% identical to the amino acid sequence set forth in any one of SEQ ID NOS: 455-464. In some embodiments, the TNFα binding protein consists of an amino acid sequence 95% identical to the amino acid sequence set forth in any one of SEQ ID NOS: 455-464. In some embodiments, the TNFα binding protein consists of an amino acid sequence 99% identical to the amino acid sequence set forth in any one of SEQ ID NOS: 455-464. In some embodiments, the TNFα binding protein consists of an amino acid sequence 100% identical to the amino acid sequence set forth in any one of SEQ ID NOS: 455-464.

In some embodiments, the TNFα binding protein comprises the amino acid sequence set forth in any one of SEQ ID NOS: 455-464, and further comprising 1 or more but less than 15% (less than 12%, less than 10%, less than 8%), amino acid variations (e.g., substitutions, additions, deletions, etc.). In some embodiments, the TNFα binding protein comprises the amino acid sequence set forth in any one of SEQ ID NOS: 455-464, and further comprising at least about 1, 2, 3, 4, 5, 6, 7, 8, 9, or 10 amino acid variations (e.g., substitutions, additions, deletions, etc.). In some embodiments, the TNFα binding protein comprises the amino acid sequence set forth in any one of SEQ ID NOS: 455-464, and further comprising about 1, 2, 3, 4, 5, 6, 7, 8, 9, or 10 amino acid variations (e.g., substitutions, additions, deletions, etc.). In some embodiments, the TNFα binding protein comprises the amino acid sequence set forth in any one of SEQ ID NOS: 455-464, and further consisting of about 1, 2, 3, 4, 5, 6, 7, 8, 9, or 10 amino acid variations (e.g., substitutions, additions, deletions, etc.). In some embodiments, the TNFα binding protein comprises the amino acid sequence set forth in any one of SEQ ID NOS: 455-464, and further comprising or consists of no more than about 1, 2, 3, 4, 5, 6, 7, 8, 9, or 10 amino acid variations (e.g., substitutions, additions, deletions, etc.).

In some embodiments, the TNFα binding protein consists of the amino acid sequence set forth in any one of SEQ ID NOS: 455-464, and further comprising 1 or more but less than 15% (less than 12%, less than 10%, less than 8%), amino acid variations (e.g., substitutions, additions, deletions, etc.). In some embodiments, the TNFα binding protein consists of the amino acid sequence set forth in any one of SEQ ID NOS: 455-464, and further comprising at least about 1, 2, 3, 4, 5, 6, 7, 8, 9, or 10 amino acid variations (e.g., substitutions, additions, deletions, etc.). In some embodiments, the TNFα binding protein consists of the amino acid sequence set forth in any one of SEQ ID NOS: 455-464, and further comprising about 1, 2, 3, 4, 5, 6, 7, 8, 9, or 10 amino acid variations (e.g., substitutions, additions, deletions, etc.). In some embodiments, the TNFα binding protein consists of the amino acid sequence set forth in any one of SEQ ID NOS: 455-464, and further consisting of about 1, 2, 3, 4, 5, 6, 7, 8, 9, or 10 amino acid variations (e.g., substitutions, additions, deletions, etc.). In some embodiments, the TNFα binding protein consists of the amino acid sequence set forth in any one of SEQ ID NOS: 455-464, and further consisting of no more than about 1, 2, 3, 4, 5, 6, 7, 8, 9, or 10 amino acid variations (e.g., substitutions, additions, deletions, etc.).

In some aspects and embodiments, a fusion protein described herein comprises a protein (or a functional fragment, functional variant, or functional fragment/variant thereof) comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 4. In some embodiments, a fusion protein described herein comprises a protein (or a functional fragment, functional variant, or functional fragment/variant thereof) comprising an amino acid sequence at least about 85% identical to the amino acid sequence of a protein set forth in Table 4. In some embodiments, a fusion protein described herein comprises a protein (or a functional fragment, functional variant, or functional fragment/variant thereof) comprising an amino acid sequence at least about 90% identical to the amino acid sequence of a protein set forth in Table 4. In some embodiments, a fusion protein described herein comprises a protein (or a functional fragment, functional variant, or functional fragment/variant thereof) comprising an amino acid sequence at least about 95% identical to the amino acid sequence of a protein set forth in Table 4. In some embodiments, a fusion protein described herein comprises a protein (or a functional fragment, functional variant, or functional fragment/variant thereof) comprising an amino acid sequence at least about 99% identical to the amino acid sequence of a protein set forth in Table 4.

In some embodiments, a fusion protein described herein comprises a protein (or a functional fragment, functional variant, or functional fragment/variant thereof) comprising an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 4. In some embodiments, a fusion protein described herein comprises a protein (or a functional fragment, functional variant, or functional fragment/variant thereof) comprising an amino acid sequence about 85% identical to the amino acid sequence of a protein set forth in Table 4. In some embodiments, a fusion protein described herein comprises a protein ((or a functional fragment, functional variant, or functional fragment/variant thereof) comprising an amino acid sequence about 90% identical to the amino acid sequence of a protein set forth in Table 4. In some embodiments, a fusion protein described herein comprises a protein (or a functional fragment, functional variant, or functional fragment/variant thereof) comprising an amino acid sequence about 95% identical to the amino acid sequence of a protein set forth in Table 4. In some embodiments, a fusion protein described herein comprises a protein (or a functional fragment, functional variant, or functional fragment/variant thereof) comprising an amino acid sequence about 99% identical to the amino acid sequence of a protein set forth in Table 4. In some embodiments, a fusion protein described herein comprises a protein (or a functional fragment, functional variant, or functional fragment/variant thereof) comprising an amino acid sequence about 100% identical to the amino acid sequence of a protein set forth in Table 4.

In some embodiments, a fusion protein described herein comprises a protein (or a functional fragment, functional variant, or functional fragment/variant thereof) consisting of an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 4. In some embodiments, a fusion protein described herein comprises a protein (or a functional fragment, functional variant, or functional fragment/variant thereof) consisting of an amino acid sequence at least about 85% identical to the amino acid sequence of a protein set forth in Table 4. In some embodiments, a fusion protein described herein comprises a protein (or a functional fragment, functional variant, or functional fragment/variant thereof) consisting of an amino acid sequence at least about 90% identical to the amino acid sequence of a protein set forth in Table 4. In some embodiments, a fusion protein described herein comprises a protein (or a functional fragment, functional variant, or functional fragment/variant thereof) consisting of an amino acid sequence at least about 95% identical to the amino acid sequence of a protein set forth in Table 4. In some embodiments, a fusion protein described herein comprises a protein (or a functional fragment, functional variant, or functional fragment/variant thereof) consisting of an amino acid sequence at least about 99% identical to the amino acid sequence of a protein set forth in Table 4.

In some embodiments, a fusion protein described herein comprises a protein (or a functional fragment, functional variant, or functional fragment/variant thereof) consisting of an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 4. In some embodiments, a fusion protein described herein comprises a protein (or a functional fragment, functional variant, or functional fragment/variant thereof) consisting of an amino acid sequence about 85% identical to the amino acid sequence of a protein set forth in Table 4. In some embodiments, a fusion protein described herein comprises a protein (or a functional fragment, functional variant, or functional fragment/variant thereof) consisting of an amino acid sequence about 90% identical to the amino acid sequence of a protein set forth in Table 4. In some embodiments, a fusion protein described herein comprises a protein (or a functional fragment, functional variant, or functional fragment/variant thereof) consisting of an amino acid sequence about 95% identical to the amino acid sequence of a protein set forth in Table 4. In some embodiments, a fusion protein described herein comprises a protein (or a functional fragment, functional variant, or functional fragment/variant thereof) consisting of an amino acid sequence about 99% identical to the amino acid sequence of a protein set forth in Table 4. In some embodiments, a fusion protein described herein comprises a protein (or a functional fragment, functional variant, or functional fragment/variant thereof) consisting of an amino acid sequence about 100% identical to the amino acid sequence of a protein set forth in Table 4.

In some embodiments, a fusion protein described herein comprises a protein (or a functional fragment, functional variant, or functional fragment/variant thereof) comprising the amino acid sequence of a protein set forth in Table 4, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 4, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 4, and further consisting of at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 4, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 4, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 4, and further comprising or no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence of a protein set forth in Table 4, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 4, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 4, and further consisting of at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 4, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 4, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 4, and further comprising or no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence at least 85% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence at least 90% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence at least 95% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence at least 99% identical to the amino acid sequence set forth in any one of SEQ ID NOS: 455-464.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence 85% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence 90% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence 95% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence 99% identical to the amino acid sequence set forth in any one of SEQ ID NOS: 455-464. In some embodiments, a fusion protein described herein comprises a protein (or a functional fragment, functional variant, or functional fragment/variant thereof) comprising an amino acid sequence 100% identical to the amino acid sequence set forth in any one of SEQ ID NOS: 455-464.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence at least 85% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence at least 90% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence at least 95% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence at least 99% identical to the amino acid sequence set forth in any one of SEQ ID NOS: 455-464.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence 85% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence 90% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence 95% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence 99% identical to the amino acid sequence set forth in any one of SEQ ID NOS: 455-464. In some embodiments, a fusion protein described herein comprises a protein (or a functional fragment, functional variant, or functional fragment/variant thereof) consisting of an amino acid sequence 100% identical to the amino acid sequence set forth in any one of SEQ ID NOS: 455-464.

In some embodiments, a fusion protein described herein comprises a protein (or a functional fragment, functional variant, or functional fragment/variant thereof) comprising the amino acid sequence set forth in any one of SEQ ID NOS: 455-464, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 455-464, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 455-464, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 455-464, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 455-464, and further comprising or consists of no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence set forth in any one of SEQ ID NOS: 455-464, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 455-464, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 455-464, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 455-464, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 455-464, and further consisting of no more than about 1, 2, 3, 4, 5, 6, 7, 8, 9, or 10 amino acid variations (e.g., substitutions, additions, deletions, etc.).

### (i) Exemplary Properties of TNFα Binding Proteins

In some embodiments, the TNFα binding proteins described herein are immunosuppressive (e.g., when administered to a subject). In some embodiments, the TNFα binding proteins described herein are anti-inflammatory (e.g., when administered to a subject). In some embodiments, the TNFα binding proteins described herein suppress pro-inflammatory response (e.g., when administered to a subject).

In some embodiments, the TNFα binding protein is capable of specifically binding TNFα. In some embodiments, the TNFα binding protein is capable of inhibiting or reducing (e.g., preventing) binding of hTNFα to hTNFR1 and/or hTNFR2.

In some embodiments, the TNFα binding proteins described herein bind a subset (e.g., one or more) TNFSF ligand. In some embodiments, the TNFα binding protein specifically binds TNFα. In some embodiments, the TNFα binding protein specifically binds hTNFα. In some embodiments, the TNFα binding protein can act as a decoy receptor for a TNFSF ligand described herein. In some embodiments, the TNFα binding protein inhibits or reduces (e.g., prevents) binding of TNFα to TNFR1. In some embodiments, the TNFα binding protein specifically binds to TNFα and inhibits or reduces (e.g., prevents) binding of TNFα to TNFR1. In some embodiments, the TNFα binding protein inhibits binding of hTNFα to hTNFR1. In some embodiments, the TNFα binding protein specifically binds to hTNFα and inhibits or reduces (e.g., prevents) binding of hTNFα to hTNFR1. In some embodiments, the TNFα binding protein inhibits or reduces (e.g., prevents) binding of TNFα to TNFR2. In some embodiments, the TNFα binding protein specifically binds to TNFα and inhibits or reduces (e.g., prevents) binding of TNFα to TNFR2. In some embodiments, the TNFα binding protein inhibits or reduces (e.g., prevents) binding of hTNFα to hTNFR2. In some embodiments, the TNFα binding protein specifically binds to hTNFα and inhibits or reduces (e.g., prevents) binding of hTNFα to hTNFR2. In some embodiments, the TNFα binding protein inhibits or reduces (e.g., prevents) binding of TNFα to TNFR1 and TNFR2. In some embodiments, the TNFα binding protein specifically binds to TNFα and inhibits or reduces (e.g., prevents) binding of TNFα to TNFR1 and TNFR2. In some embodiments, the TNFα binding protein inhibits or reduces (e.g., prevents) binding of hTNFα to hTNFR1 and TNFR2. In some embodiments, the TNFα binding protein specifically binds to hTNFα and inhibits or reduces (e.g., prevents) binding of hTNFα to hTNFR1 and TNFR2. In some embodiments, the TNFα binding protein inhibits NF-κB signaling mediated by binding of hTNFα to hTNFR1. In some embodiments, the TNFα binding protein inhibits NF-κB signaling mediated by binding of hTNFα to hTNFR2.

In some embodiments, the TNFα binding protein specifically binds LTα. In some embodiments, the TNFα binding protein specifically binds hLTα. In some embodiments, the TNFα binding protein inhibits or reduces (e.g., prevents) binding of LTα to TNFR1. In some embodiments, the TNFα binding protein specifically binds to LTα and inhibits or reduces (e.g., prevents) binding of LTα to TNFR1. In some embodiments, the TNFα binding protein inhibits or reduces (e.g., prevents) binding of hLTα to hTNFR1. In some embodiments, the TNFα binding protein specifically binds to hLTα and inhibits or reduces (e.g., prevents) binding of hLTα to hTNFR1. In some embodiments, the TNFα binding protein inhibits or reduces (e.g., prevents) binding of LTα to TNFR2. In some embodiments, the TNFα binding protein specifically binds to LTα and inhibits or reduces (e.g., prevents) binding of LTα to TNFR2. In some embodiments, the TNFα binding protein inhibits or reduces (e.g., prevents) binding of hLTα to hTNFR2. In some embodiments, the TNFα binding protein specifically binds to hLTα and inhibits or reduces (e.g., prevents) binding of hLTα to hTNFR2. In some embodiments, the TNFα binding protein inhibits or reduces (e.g., prevents) binding of LTα to TNFR1 and TNFR2. In some embodiments, the TNFα binding protein specifically binds to LTα and inhibits or reduces (e.g., prevents) binding of LTα to TNFR1 and TNFR2. In some embodiments, the TNFα binding protein inhibits or reduces (e.g., prevents) binding of hLTα to hTNFR1 and TNFR2. In some embodiments, the TNFα binding protein specifically binds to hLTα and inhibits or reduces (e.g., prevents) binding of hLTα to hTNFR1 and TNFR2. In some embodiments, the TNFα binding protein inhibits or reduces (e.g., prevents) NF-κB signaling mediated by binding of hLTα to hTNFR1. In some embodiments, the TNFα binding protein inhibits or reduces (e.g., prevents) NF-κB signaling mediated by binding of hLTα to hTNFR2.

### 5.2.1.4 CD30 Ligand Binding Proteins

In some aspects and embodiments, a fusion protein described herein (or a combination regimen described herein) comprises a protein that specifically binds the hCD30L.

The amino acid sequence of proteins capable of specifically binding hCD30L is set forth in Table 5 (SEQ ID NOS: 465-576). The amino acid sequence of the mature form of the proteins (i.e., lacking the native signal peptide) is set forth in SEQ ID NOS: 465-522. The amino acid sequence of the immature form of the proteins (i.e., containing the native signal peptide) is set forth in SEQ ID NOS: 523-576.

The signal peptides have been computationally predicted using standard methods (see, e.g., Teufel 2022). A person of ordinary skill in the art would know how to experimentally identify and/or validate a computationally predicted signal peptide using standard methods known in the art, e.g., expression of a protein from a host cell and sequencing of the intracellular form and the extracellular form of the expressed protein (see, e.g., Zhang 2004).

TABLE 5
The Amino Acid Sequence of CD30L Targeting Proteins.

| | | SEQ ID |
|---|---|---|
| Description | Amino Acid Sequence | NO |
| IMP-215 | KTCPADYYLEPEDGLCTACVTCLSNMVEIQPCGPDKPRKCQCGPGL | 465 |
| without native signal | KCTLPAVNSCARCTPDTTTKKVQKEQCCTTPDNTKLCYHKYS | |
| peptide | | |
| IMP-216 | KTCPADYYLEPEDGLCTACVTCLSNMVEIQPCGPDKPRKCQCGPGL | 466 |
| without native signal | KCTVPAVNSCARCTPDTTTKKIEPIGQCCTTPDNTKLCYHKYS | |
| peptide | | |
| IMP-217 | KTCPADYYLNPENGLCTACVTCLSNMVEIQPCGPDKPRKCECGSGF | 467 |
| without native signal | KCTLPAVNSCARCTPDTTTKKVQKEQKEQCCNTPDNTKLCYHKYSS | |
| peptide | | |
| IMP-218 | KTCPNDYYLEPEDGLCTACVTCLSNMVEIQPCGPDKPRKCKCGPGL | 468 |
| without native signal | KCTVPAVNSCARCTPDTTTKKIEPIGQCCTTPDNTKLCYHKYS | |
| peptide | | |
| IMP-219 | KTCPNDYYLEPEDGLCTACVTCLSNMVEIQPCGPDKPRKCQCGPGL | 469 |
| without native signal | KCTVPAVNSCARCTPDTTTKKIEPIGQCCTTPDNTKLCYHKYS | |
| peptide | | |
| IMP-220 | KTCPADYYLEPEDGLCTACVTCLSNMVETQSCGPDKPRKCQCGPGL | 470 |
| without native signal | KCTVPAVNSCARCTPDTTTKKVQKEQCCTTPDNTKLCYHKYS | |
| peptide | | |
| IMP-221 | TCPNDYYLEPEDGLCTACVTCLSNMVEIQPCGPDKPRKCQCGPGLK | 471 |
| without native signal | CTVPAVNSCARCTPDTTIKKIKPTDQCCTTPDNTKLCYHK | |
| peptide | | |
| IMP-222 | TCPNDYYLEPEDGLCTACVTCLSNMVEIQPCGPDKPRKCQCGPGLK | 472 |
| without native signal | CTVPAVNSCARCTPDTTIKKIEPTDQCCTTPDNTKLCYHKYSP | |
| peptide | | |
| IMP-223 | TCPADYYLEPEDGLCTACVTCLSNMVEIQPCGPDKPRKCQCGPGLK | 473 |
| without native signal | CTVPAVNSCARCTPDTTTKKVQKDQCCTTPDNTKLCYHKYSS | |
| peptide | | |
| IMP-224 | TCPVDHYYDREDGGLCTACVTCLQNMVEIQPCGPDKPRKCQCAPGF | 474 |
| without native signal | KCTLPAVNSCARCTPDSTYKPPPKPATKPKSHEDNCCITTGNTKLC | |
| peptide | YTQLN | |
| IMP-225 | DCEKDYYRDPKTGRCTACVTCTGDTVEKSPCGPTTPRKCECGPGLK | 475 |
| without native signal | CTVSATNTCARCEEHIPDNCCKTKDNTKLCYQKLRGS | |
| peptide | | |
| IMP-226 | TCPADYYLEPEDGLCTACVTCLSNMVETQSCGPDKPRKCQCGPGLK | 476 |
| without native signal | CTVPAVNSCARCTPDTTTKKVQKEQCCTTPDNTKLCYHKYSS | |
| peptide | | |
| IMP-227 | MKCEQCVSYYNTQELKCCKLSKPGTYSDHRCDKYSDTICGHCPSDT | 477 |
| without native signal | FTSIYNRSPRCHSCRGHTLYTYH | |
| peptide | | |
| IMP-228 | MKCEQCVSYYNTQELKCCKLSKPGTYSDHRCDKYSDTICGHCPSDT | 478 |
| without native signal | FTSIYNRSPRCYSCRGHTLYTYH | |
| peptide | | |
| IMP-229 | TCTEGSFLNSLDKKCYDCPYNTWQASRRHSEIVCNMCTQCKPGYTL | 479 |
| without native signal | TQKCTPTTDTVCMCRKPFVEKNGFCMLFHKP | |
| peptide | | |
| IMP-230 | YYKDPTTNRCTACVTCGGDMVETAPCGPNTPRKCECGPGLKCALAV | 480 |
| without native signal | QNSCARCEEYIPDNCCKTKDKTKLCYEKLRGS | |
| peptide | | |
| IMP-231 | TCPNDYYLEPEDGLCKACVTCLSNMVEIQPCGPDKPRKCQCGPGLK | 481 |
| without native signal | CTVPVVNSCARCTPDTTTKKVQKEQCCTTPDNTKLCYHKYSS | |
| peptide | | |
| IMP-323 | TCPADYYLEPEDGLCTACVTCLSNMVEIQPCGPDKPRKCQCGPGLK | 482 |
| without native signal | CTVPAVNSCARCTPDTTTKKVQKEQCCTTPDNTKLCYHKYSS | |
| peptide | | |
| IMP-233 | MVCCRLYPAGSYAEQLCTKDNDTVCNPCATETFLSIPNYTSKCLSC | 483 |
| without native signal | RGKCTKDQVEVRPCTITRNRTCKCKDGYICILKTDDNSCRVC | |
| peptide | | |
| IMP-234 | TCPNDYYLEPEDGLCTACVTCLSNMVEIQPCGPNKPRKCQCGPGLK | 484 |
| without native signal | CTVPAVNSCARCTPDTTIKKIEPTDQCCTTPDNTKLCYHKYSS | |
| peptide | | |
| IMP-235 | TCPNDYYLEPEDGLCTACVTCLSNMVEIQPCGPDKPRKCQCGPGLK | 485 |
| without native signal | CTVPAVNSCARCTPDTTIKKIEPTDQCCTTPDNTKLCYHKYSS | |
| peptide | | |
| IMP-236 | TCPNDYYLEPEDGLCKACVTCLSNMVEIQSCGPDKPRKCQCGPGLK | 486 |
| without native signal | CMLPAVNSCARCTPDTTTKKIEPTEQCCTTPDNTKLCYHKYSS | |
| peptide | | |
| IMP-237 | TCPADYYLEPEDGLCTACVTCLSNMVETQPCGPDKPRKCQCGPGLK | 487 |
| without native signal | CTLPAVNSCARCTPDTTTKKVQKEQCCTTPDNTKLCYHKYSSS | |
| peptide | | |
| IMP-238 | TCPNDYYLEPEDGLCTACVTCLSNMVEIQPCGPDKPRKCQCGPGLK | 488 |
| without native signal | CTVPAVNSCARCTPDTTTKKIEPIGQCCTTPDNTKLCYHKYSS | |
| peptide | | |
| IMP-239 | TCPNDYYLEPEDGLCKACVTCLSNMVEIQSCGPDKPRKCQCGPGLK | 489 |
| without native signal | CTLPAVNSCARCTPDTTTKKIEPTEQCCTTPDNTKLCYHKYSS | |
| peptide | | |
| IMP-240 | TCPADYYLEPEDGLCTACVTCLSNMVEIQPCGPDKPRKCQCGPGLK | 490 |
| without native signal | CTVPAVNSCARCTPDTTTKKIEPIGQCCTTPDNTKLCYHKYSS | |
| peptide | | |
| IMP-241 | TCPNDYYLEPEDGLCTACVTCLSNMVEIQPCGPDKPRKCKCGPGLK | 491 |
| without native signal | CTVPAVNSCARCTPDTTTKKIEPIGQCCTTPDNTKLCYHKYSS | |
| peptide | | |
| IMP-242 | YASRLCDSKTNTQCTPCGSGTFTSRNNHLPACLSCNGRCDSNQVET | 492 |
| without native signal | RSCNTTHNRICECSPGYYCILKGSSGCKACVSQNKCGIGYGVS | |
| peptide | | |
| IMP-243 | TCPADYYLNPENGLCTACVTCLSNMVEIQPCGPDKPRKCECGSGFK | 493 |
| without native signal | CTLPAVNSCARCTPDTTTKKVQKEQKEQCCNTPDNTKLCYHKYSS | |
| peptide | | |
| IMP-244 | KICQNDYYFNKETGHCTACVTCVGNTKETHPCGPDEPRTCECDSGF | 494 |
| without native signal | KCDLPVANSCARCIVIPPTKSTKNKHIKEQCCNTTDNVKLCYTIQN | |
| peptide | SFIK | |
| IMP-245 | TCPTDYYYNREDDGLCTACVTCLKNMVEIQPCGPDKPRKCQCAPGF | 495 |
| without native signal | KCTVPAVNSCARCTPDSTYKPPIPKPASKPKSPDDNCCVTTSNIKL | |
| peptide | CYTQLN | |
| IMP-246 | DIAPHAPSDGKCKDNEYKRHNLCPGTYASRLCDSKTNTQCTPCTFT | 496 |
| without native signal | SRNNHLPACLSCNGRRDRVTRLTIESVNALPDIIVFSKDHPDARHV | |
| peptide | FPKQNVE | |
| IMP-247 | DIAPHAPSDGKCKDNEYKRHNLCPGTYASDSKTNTRCTPCGSGTFT | 497 |
| without native signal | SRNNHLPACLSCNGRRDRVTRLTIESVNALPDIIVFSKDHPDARHV | |
| peptide | FPKQNVE | |
| IMP-248 | RDIAPHAPSDGKCKDNEYKRHNLCPGTYASRLCDSKTNTQCTPCTF | 498 |
| without native signal | TSRNNHLPACLSCNGRRDRVTRLTIESVNALPDIIVFSKDHPDARH | |
| peptide | VFPKQNV | |
| IMP-249 | RDIAPHAPSDGKCKDNEYKRHNLCPGTYASRLCDSKTNTQCTPCTF | 499 |
| without native signal | TSRNNHLPACLSCNGRRDRVTLLTIESVNALPDIIVFSKDHPDARH | |
| peptide | VFPKQNV | |
| IMP-250 | EVKLGNQCCPPCGSGQKVTKVCTENSGITCTLCPNGTYLTGLYNCT | 500 |
| without native signal | NCTQCNDTQITVRNCTSTNNTICASKNHTLFSTPGVQHHKQRQQNH | |
| peptide | TAHVTVKQ | |
| IMP-251 | DIAPHAPSNGKCKDNEYKRHNLCPGTYASRLCDSKTNTRCTPCGSG | 501 |
| without native signal | TFTSRNNHLPACLSCNGRRDRVTLLTIESVNALPDIIVFSKDHPDA | |
| peptide | RHVFPKQNVE | |
| IMP-252 | DIAPHAPSDGKCKDNEYKRHNLCPGTYASRLCDSKTNTQCTPCGSG | 502 |
| without native signal | TFTSRNNHLPACLSCNGRRDRVTRLTIESVNALPDIIVFSKDHPDA | |
| peptide | RHVFPKQNVE | |
| IMP-253 | DIAPHAPSDGKCKDNEYKRHNLCPGTYASRLCDSKTNTRCTPCGSG | 503 |
| without native signal | TFTSRNNHLPACLSCNGRRDRVTRLTIESVNALPDIIVFSKDHPDA | |
| peptide | RHVFPKQNVE | |
| IMP-254 | RDIAPHAPSDGKCKDNEYKRHNLCPGTYASRLCDSKTNTQCTPCGS | 504 |
| without native signal | GTFTSRNNHLPACLSCNGRRDRVTRLTIESVNALPDIIVFSKDHPD | |
| peptide | ARHVFPKQNV | |
| IMP-255 | RDIAPHAPSDGKCKDNEYKRHNLCPGTYASRLCDSKTNTQCTPCGS | 505 |
| without native signal | GTFTSRNNHLPACLSCNGRCDRVTLLTIESVNALPDIIVFSKDHPD | |
| peptide | ARHVFPKQNV | |
| IMP-256 | RDIAPHAPSNGKCKDNEYKRHNLCPGTYASRLCDSKTNTRCTPCGS | 506 |
| without native signal | GTFTSRNNHLPACLSCNGRRDRVTRLTIESVNALPDIIVFSKDHPD | |
| peptide | ARHVFPKQNV | |
| IMP-257 | RDTAPHAPSDGKCKGNEYKRHNLWPGTYASRLCDSKTNTQCTPCGS | 507 |
| without native signal | GTFTSRNNHLPARLSCNGRRDRVTRLTIESVDALPDIIVFSKDHPD | |
| peptide | ARHVFPKQNV | |
| IMP-258 | RDIAPHAPSDGKCKDNEYKRHNLCPGTYASRLCDSKTNTQCTPCGS | 508 |
| without native signal | GTFTSRNNHLPACLSCNGRRDRVTLLTIESVNALPDIIVSKDHPD | |
| peptide | ARHVFPKPNV | |
| IMP-259 | RDIAPHAPSDGKCKDNEYKRHNLCPGTYASRLCDSKTNTRCTPCGS | 509 |
| without native signal | GTFTSRNNHLPACLSCNGRRDRVTLLTIESVNALPDIIVFSKDHPD | |
| peptide | ARHVFPKQNV | |
| IMP-260 | DNNCGELEYYNKVHDVCCKLCPAGFYAKQLCTKDMDTVCNPCATET | 510 |
| without native signal | FLSIPNYTSKCLSCRGKCTKDQVEVRPCTITRNRTCKCKDGYICIL | |
| peptide | KTDDNSCRVCV | |
| IMP-261 | RDIAPHAPSNGKCKDNEYKRHNLCCLSCPPGTYASRLCDSKTNTQC | 511 |
| without native signal | TPCGSGTFTSRNNHLPACLSCNGRRDRVTLLTIESVNALPDIIVFS | |
| peptide | KDHPDARHVFPKQNV | |
| IMP-262 | PTRHSSCGETEYYETELQKCCQKCQPGTKFKSVCTKDSPTVCEACV | 512 |
| without native signal | AGSYSATHNYYENCFSCDKCSGGRKVVKEQCTPTKNTVCGCQGDYE | |
| peptide | MKTVGGTNMCRKPKRS | |
| IMP-263 | PTRHSSCGETEYYETELQKCCQKCQPGTKFKSVCTKDSPTVCEACV | 513 |
| without native signal | AGSTYSATHNYYENCFSCDKCSGGRKVVKEQCTPTKNTVCGCQGDY | |
| peptide | EMKTVGGTNMCRKPKRS | |
| IMP-264 | KPTRHSSCGETEYYETELQKCCQKCQPGTKFKSVCTKDSPTVCEAC | 514 |
| without native signal | VAGSTYSATHNYYENCFSCDKCSGGRKVVKEQCTPTKNTVCGCQGD | |
| peptide | YEMKTVGGTNMCRKPKR | |
| IMP-265 | IPTSSLPHAPVNGACDEGEYLDKRHNQCCNQCPPGEFAKVRCNGND | 515 |
| without native signal | NTKCERCPPHTYTAIPNYSNGCHQCRKCPTGSFDKVKCTGTQNSKC | |
| peptide | SCLPGWYCATDSSQTEDC | |
| IMP-266 | VCQHNEVQLGNECCPPCGSGQRVTKVCTDYTSVTCTPCPNGTYVSG | 516 |
| without native signal | PYNCTDCTQCNVTQVMIRNCTSTNNTVCAPKNHTYFPTPGVQHHKQ | |
| peptide | RQQNHTAHITVKQGKSGRHTLA | |
| IMP-267 | IPTSSLPHAPVNGACDEGEYLDKRHNQCCNRCPPGEFAKVRCNGND | 517 |
| without native signal | NTKCERCPPHTYTAIPNYSNGCHQCRKCPTGSFDKVKCTGTQNSKC | |
| peptide | SCLPGWYCATDSSQTEDCRDCIPKRKMSMRILWWNR | |
| IMP-268 | DIPTTPHSPINGSCDIGEYLDKKSGNCCKMCPPGAFAKVRCTSDNN | 518 |
| without native signal | TECANCPPGTFTSIPNYSNGCHQCRGHCPEGAFDEKRCTTTHDRIC | |
| peptide | KCLPGWFCVTGSASLQCPMCIPKRKCPCGYFGGIDELGNPLCKSCC | |
| | KGEYCEHIRSYRPGYVPCNLSKCN | |
| IMP-269 | IHTPLPPHASVNGSCAEGEYLDKTHNRCCNRCPPGEFAKVRCSGSY | 519 |
| without native signal | NTKCERCPHHTYTAIPNYSNGCHQCRKCPKGSFDKVKCTGTQNSKC | |
| peptide | ACLPGWYCATDSSQTEDCRDCVPKRRCPCGYFGGIDKLGNPICKSC | |
| | CVGKYCDDIRNHRVGPFPPCKLSKCN | |
| IMP-270 | IPTSSLPHAPVNGACDEGEYLDKRHNQCCNQCPPGEFAKVRCNGND | 520 |
| without native signal | NTKCERCPPHTYTAIPNYSNGCHQCRKCPTGSFDKVKCTGTQNSKC | |
| peptide | SCLPGWYCATDSSQTEDCRDCIPKRRCPCGYFGGIDEQGNPICKSC | |
| | CVGEYCDYLRNYRLDPFPPCKLSKCN | |
| IMP-271 | IPTPPLPHAPVNGACDEGEYLDKRHNQCCNQCPPGEFAKVRCNGND | 521 |
| without native signal | NTKCERCPPHTYTAIPNYSNGCHQCRKCPTGSFDKVKCTGTQNSKC | |
| peptide | SCLPGWYCATDSSQTEDCRDCIPKRRCPCGYFGGIDEQGNPICKSC | |
| | CVGEYCDYLRNYRLDPFPPCKLSKCN | |
| IMP-272 | ITPHEPSNGKCKDNEYKHHHLCCLSCPPGTYASRLCDSKTNTNTQC | 522 |
| without native signal | TPCGSGTFTSRNNHLPACLSCNGRCDSNQVETRSCNTTHNRICDCA | |
| peptide | PGYYCLLKGSSGCKACVSQTKCGIGYGVSGHTPTGDVVCSPCGLGT | |
| | YSHTVSSVDKCEPVPSNTFNYIDVEINLYPVNDTSCTRTTTTGLSE | |
| | SISTSELTITMNHKDCDPVFRDGYFSVLNKVATSGFFTGQNRYQNI | |
| | SKVCTLNFEIKCNNKDSYSSSKQLTKAKNDDDSIMPHSETVTLVGD | |
| | CLSSVDIYILYSNTNTQDYETDTISYHVGNVLDVDSHMPGSCDIHK | |
| | LITNSSHSLL | |
| IMP-215 | MNIIFLSAIVTCLVYTTFGKTCPADYYLEPEDGLCTACVTCLSNMV | 523 |
| with native signal | EIQPCGPDKPRKCQCGPGLKCTLPAVNSCARCTPDTTTKKVQKEQC | |
| peptide | CTTPDNTKLCYHKYS | |
| IMP-216 | MTIHILDVLNSMKMNTIFLSAIVTCIVYTTFGKTCPADYYLEPEDG | 524 |
| with native signal | LCTACVTCLSNMVEIQPCGPDKPRKCQCGPGLKCTVPAVNSCARCT | |
| peptide | PDTTTKKIEPIGQCCTTPDNTKLCYHKYS | |
| IMP-217 | MNIIFLSAIVSCLVYTTFGGKTCKTCPADYYLNPENGLCTAC | 525 |
| with native signal | VTCLSNMVEIQPCGPDKPRKCECGSGFKCTLPAVNSCARCTP | |
| peptide | DTTTKKVQKEQKEQCCNTPDNTKLCYHKYSS | |
| IMP-218 | MKMNTIFLSAIVTCIVYTSFGKTCPNDYYLEPEDGLCTACVT | 526 |
| with native signal | CLSNMVEIQPCGPDKPRKCKCGPGLKCTVPAVNSCARCTPDT | |
| peptide | TTKKIEPIGQCCTTPDNTKLCYHKYS | |
| IMP-219 | MKMNTIFLSAIVTCLVYTSFGKTCPNDYYLEPEDGLCTACVT | 527 |
| with native signal | CLSNMVEIQPCGPDKPRKCQCGPGLKCTVPAVNSCARCTPDT | |
| peptide | TTKKIEPIGQCCTTPDNTKLCYHKYS | |
| IMP-220 | MKMNIIFLSAIVTCLVYTTFGKTCPADYYLEPEDGLCTACVT | 528 |
| with native signal | CLSNMVETQSCGPDKPRKCQCGPGLKCTVPAVNSCARCTPDT | |
| peptide | TTKKVQKEQCCTTPDNTKLCYHKYS | |
| IMP-221 | MKMNTIFLSAIVTCIVYTSFGKTCPNDYYLEPEDGLCTACVT | 529 |
| with native signal | CLSNMVEIQPCGPDKPRKCQCGPGLKCTVPAVNSCARCTPDT | |
| peptide | TIKKIKPTDQCCTTPDNTKLCYHK | |
| IMP-222 | MKMNTIFLSAIVTCLVYTSFGKTCPNDYYLEPEDGLCTACVT | 530 |
| with native signal | CLSNMVEIQPCGPDKPRKCQCGPGLKCTVPAVNSCARCTPDT | |
| peptide | TIKKIEPTDQCCTTPDNTKLCYHKYSP | |
| IMP-223 | MKMNTIFLSAIVTCIVYTSFGKTCPADYYLEPEDGLCTACVT | 531 |
| with native signal | CLSNMVEIQPCGPDKPRKCQCGPGLKCTVPAVNSCARCTPDT | |
| peptide | TTKKVQKDQCCTTPDNTKLCYHKYSS | |
| IMP-224 | MNKFIVLSTIVSCLVYTSFGKTCPVDHYYDREDGGLCTACVT | 532 |
| with native signal | CLQNMVEIQPCGPDKPRKCQCAPGFKCTLPAVNSCARCTPDS | |
| peptide | TYKPPPKPATKPKSHEDNCCITTGNTKLCYTQLN | |
| IMP-225 | MKYILLITICYSLYCISFASDCEKDYYRDPKTGRCTACVTCT | 533 |
| with native signal | GDTVEKSPCGPTTPRKCECGPGLKCTVSATNTCARCEEHIPD | |
| peptide | NCCKTKDNTKLCYQKLRGS | |
| IMP-226 | MKMNIIFLSAIVTCLVYTTFGKTCPADYYLEPEDGLCTACVT | 534 |
| with native signal | CLSNMVETQSCGPDKPRKCQCGPGLKCTVPAVNSCARCTPDT | |
| peptide | TTKKVQKEQCCTTPDNTKLCYHKYSS | |
| IMP-229 | MKPQILFLCACVILMASNKKADARTCTEGSFLNSLDKKCYDC | 535 |
| with native signal | PYNTWQASRRHSEIVCNMCTQCKPGYTLTQKCTPTTDTVCMC | |
| peptide | RKPFVEKNGFCMLFHKP | |
| IMP-230 | MKYILLITICYSLYCISFASQCEADYYKDPTTNRCTACVTCG | 536 |
| with native signal | GDMVETAPCGPNTPRKCECGPGLKCALAVQNSCARCEEYIPD | |
| peptide | NCCKTKDKTKLCYEKLRGS | |
| IMP-231 | MKMNTIFLSAIVTCLVYTSFGKTCPNDYYLEPEDGLCKACVT | 537 |
| with native signal | CLSNMVEIQPCGPDKPRKCQCGPGLKCTVPVVNSCARCTPDT | |
| peptide | TTKKVQKEQCCTTPDNTKLCYHKYSS | |
| IMP-232 | MKMNIIFLSAIVTCLVYTTFGKTCPADYYLEPEDGLCTACVT | 538 |
| with native signal | CLSNMVEIQPCGPDKPRKCQCGPGLKCTVPAVNSCARCTPDT | |
| peptide | TTKKVQKEQCCTTPDNTKLCYHKYSS | |
| IMP-234 | MKMNTIFLSAIVTCLVYTSFGKTCPNDYYLEPEDGLCTACVT | 539 |
| with native signal | CLSNMVEIQPCGPNKPRKCQCGPGLKCTVPAVNSCARCTPDT | |
| peptide | TIKKIEPTDQCCTTPDNTKLCYHKYSS | |
| IMP-235 | MKMNTIFLSAIVTCLVYTSFGKTCPNDYYLEPEDGLCTACVT | 540 |
| with native signal | CLSNMVEIQPCGPDKPRKCQCGPGLKCTVPAVNSCARCTPDT | |
| peptide | TIKKIEPTDQCCTTPDNTKLCYHKYSS | |
| IMP-236 | MKMNTIFLSAIVTCLVYTSFGKTCPNDYYLEPEDGLCKACVT | 541 |
| with native signal | CLSNMVEIQSCGPDKPRKCQCGPGLKCMLPAVNSCARCTPDT | |
| peptide | TTKKIEPTEQCCTTPDNTKLCYHKYSS | |
| IMP-237 | MNIIFLSAIVTCLVYTTFGKTCPADYYLEPEDGLCTACVTCL | 542 |
| with native signal | SNMVETQPCGPDKPRKCQCGPGLKCTLPAVNSCARCTPDTTT | |
| peptide | KKVQKEQCCTTPDNTKLCYHKYSSS | |
| IMP-238 | MNTIFLSAIVTCLVYTSFGKTCPNDYYLEPEDGLCTACVTCL | 543 |
| with native signal | SNMVEIQPCGPDKPRKCQCGPGLKCTVPAVNSCARCTPDTTT | |
| peptide | KKIEPIGQCCTTPDNTKLCYHKYSS | |
| IMP-239 | MKMNTIFLSAIVTCLVYTSFGKTCPNDYYLEPEDGLCKACVT | 544 |
| with native signal | CLSNMVEIQSCGPDKPRKCQCGPGLKCTLPAVNSCARCTPDT | |
| peptide | TTKKIEPTEQCCTTPDNTKLCYHKYSS | |
| IMP-240 | MNTIFLSAIVTCIVYTTFGKTCPADYYLEPEDGLCTACVTCL | 545 |
| with native signal | SNMVEIQPCGPDKPRKCQCGPGLKCTVPAVNSCARCTPDTTT | |
| peptide | KKIEPIGQCCTTPDNTKLCYHKYSS | |
| IMP-241 | MNTIFLSAIVTCIVYTSFGKTCPNDYYLEPEDGLCTACVTCL | 546 |
| with native signal | SNMVEIQPCGPDKPRKCKCGPGLKCTVPAVNSCARCTPDTTT | |
| peptide | KKIEPIGQCCTTPDNTKLCYHKYSS | |
| IMP-243 | MNIIFLSAIVSCLVYTTFGKTCPADYYLNPENGLCTACVTCLSNMV | 547 |
| with native signal | EIQPCGPDKPRKCECGSGFKCTLPAVNSCARCTPDTTTKKVQKEQK | |
| peptide | EQCCNTPDNTKLCYHKYSS | |
| IMP-244 | MGYKGIYKIIFFSTIMYSLVYCKICQNDYYFNKETGHCTACVTCVG | 548 |
| with native signal | NTKETHPCGPDEPRTCECDSGFKCDLPVANSCARCIVIPPTKSTKN | |
| peptide | KHIKEQCCNTTDNVKLCYTIQNSFIK | |
| IMP-245 | MNKFIIVYTIVSYLVYTSFGKTCPTDYYYNREDDGLCTACVTCLKN | 549 |
| with native signal | MVEIQPCGPDKPRKCQCAPGFKCTVPAVNSCARCTPDSTYKPPIPK | |
| peptide | PASKPKSPDDNCCVTTSNIKLCYTQLN | |
| IMP-246 | MKSVLYSYILFLSCIIINGRDIAPHAPSDGKCKDNEYKRHNLCPGT | 550 |
| with native signal | YASRLCDSKTNTQCTPCTFTSRNNHLPACLSCNGRRDRVTRLTIES | |
| peptide | VNALPDIIVFSKDHPDARHVFPKQNVE | |
| IMP-247 | MKSVLYSYILFLSCIIINGRDIAPHAPSDGKCKDNEYKRHNLCPGT | 551 |
| with native signal | YASDSKTNTRCTPCGSGTFTSRNNHLPACLSCNGRRDRVTRLTIES | |
| peptide | VNALPDIIVFSKDHPDARHVFPKQNVE | |
| IMP-248 | MKSVLYLYILFLSCIIINGRDIAPHAPSDGKCKDNEYKRHNLCPGT | 552 |
| with native signal | YASRLCDSKTNTQCTPCTFTSRNNHLPACLSCNGRRDRVTRLTIES | |
| peptide | VNALPDIIVFSKDHPDARHVFPKQNV | |
| IMP-249 | MIERKYLYDWRSRKQEQDDDYYIIKSVLYSYILFLSCIIINGRDIA | 553 |
| with native signal | PHAPSDGKCKDNEYKRHNLCPGTYASRLCDSKTNTQCTPCTFTSRN | |
| peptide | NHLPACLSCNGRRDRVTLLTIESVNALPDIIVFSKDHPDARHVFPK | |
| | QNV | |
| IMP-250 | MKPLMMLICFGVFLLQLGGSKMCKPDEVKLGNQCCPPCGSGQKVTK | 554 |
| with native signal | VCTENSGITCTLCPNGTYLTGLYNCTNCTQCNDTQITVRNCTSTNN | |
| peptide | TICASKNHTLFSTPGVQHHKQRQQNHTAHVTVKQ | |
| IMP-251 | MKSVLYSYILFLSCIIINGRDIAPHAPSNGKCKDNEYKRHNLCPGT | 555 |
| with native signal | YASRLCDSKTNTRCTPCGSGTFTSRNNHLPACLSCNGRRDRVTLLT | |
| peptide | IESVNALPDIIVFSKDHPDARHVFPKQNVE | |
| IMP-252 | DIAPHAPSDGKCKDNEYKRHNLCPGTYASRLCDSKTNTQCTPCGSG | 556 |
| with native signal | MKSVLYSYILFLSCIIINGRTFTSRNNHLPACLSCNGRRDRVTRLT | |
| peptide | IESVNALPDIIVFSKDHPDARHVFPKQNVE | |
| IMP-253 | MKSVLYSYILFLSCIIINGRDIAPHAPSDGKCKDNEYKRHNLCPGT | 557 |
| with native signal | YASRLCDSKTNTRCTPCGSGTFTSRNNHLPACLSCNGRRDRVTRLT | |
| peptide | IESVNALPDIIVFSKDHPDARHVFPKQNVE | |
| IMP-254 | MKSVLYSYILFLSCIIINGRDIAPHAPSDGKCKDNEYKRHNLCPGT | 558 |
| with native signal | YASRLCDSKTNTQCTPCGSGTFTSRNNHLPACLSCNGRRDRVTRLT | |
| peptide | IESVNALPDIIVFSKDHPDARHVFPKQNV | |
| IMP-255 | MKSVLYLYILFLSCIIINGRDIAPHAPSDGKCKDNEYKRHNLCPGT | 559 |
| with native signal | YASRLCDSKTNTQCTPCGSGTFTSRNNHLPACLSCNGRCDRVTLLT | |
| peptide | IESVNALPDIIVFSKDHPDARHVFPKQNV | |
| IMP-256 | MKSVLYSYILFLSCIIINGERDIAPHAPSNGKCKDNEYKRHNLCPG | 560 |
| with native signal | TYASRLCDSKTNTRCTPCGSGTFTSRNNHLPACLSCNGRRDRVTRL | |
| peptide | TIESVNALPDIIVFSKDHPDARHVFPKQNV | |
| IMP-257 | MKSVLYSYILFLSFIIINGRDTAPHAPSDGKCKGNEYKRHNLWPGT | 561 |
| with native signal | YASRLCDSKTNTQCTPCGSGTFTSRNNHLPARLSCNGRRDRVTRLT | |
| peptide | IESVDALPDIIVFSKDHPDARHVFPKQNV | |
| IMP-258 | MKSVLYLYILFLSCIIINGRDIAPHAPSDGKCKDNEYKRHNLCPGT | 562 |
| with native signal | YASRLCDSKTNTQCTPCGSGTFTSRNNHLPACLSCNGRRDRVTLLT | |
| peptide | IESVNALPDIIVFSKDHPDARHVFPKPNV | |
| IMP-259 | MIERKYLYDWRSRKQEQDNDYYIIKSVLYSYILFLSCIIINGRDIA | 563 |
| with native signal | PHAPSDGKCKDNEYKRHNLCPGTYASRLCDSKTNTRCTPCGSGTFT | |
| peptide | SRNNHLPACLSCNGRRDRVTLLTIESVNALPDIIVFSKDHPDARHV | |
| | FPKQNV | |
| IMP-260 | MYKHCYYILYLFISLTVVCSSDNNCGELEYYNKVHDVCCKLCPAGF | 564 |
| with native signal | YAKQLCTKDMDTVCNPCATETFLSIPNYTSKCLSCRGKCTKDQVEV | |
| peptide | RPCTITRNRTCKCKDGYICILKTDDNSCRVCV | |
| IMP-261 | MKSVLYLYILFLSCIIINGRDIAPHAPSNGKCKDNEYKRHNLCCLS | 565 |
| with native signal | CPPGTYASRLCDSKTNTQCTPCGSGTFTSRNNHLPACLSCNGRRDR | |
| peptide | VTLLTIESVNALPDIIVFSKDHPDARHVFPKQNV | |
| IMP-262 | MKLLVILGLGLCYLAVQAKPTRHSSCGETEYYETELQKCCQKCQPG | 566 |
| with native signal | TKFKSVCTKDSPTVCEACVAGSYSATHNYYENCFSCDKCSGGRKVV | |
| peptide | KEQCTPTKNTVCGCQGDYEMKTVGGTNMCRKPKRS | |
| IMP-263 | MKLLVILGLGLCYLAVQAKPTRHSSCGETEYYETELQKCCQKCQPG | 567 |
| with native signal | TKFKSVCTKDSPTVCEACVAGSTYSATHNYYENCFSCDKCSGGRKV | |
| peptide | VKEQCTPTKNTVCGCQGDYEMKTVGGTNMCRKPKRS | |
| IMP-264 | MTHTVTSAECLLLSVSVLNKISCFTILYHISDLISSQTMKLLVILG | 568 |
| with native signal | LGLCYLAVQAKPTRHSSCGETEYYETELQKCCQKCQPGTKFKSVCT | |
| peptide | KDSPTVCEACVAGSTYSATHNYYENCFSCDKCSGGRKVVKEQCTPT | |
| | KNTVCGCQGDYEMKTVGGTNMCRKPKR | |
| IMP-265 | MDIKNLLTACTIFYITTLATADIPTSSLPHAPVNGACDEGEYLDKR | 569 |
| with native signal | HNQCCNQCPPGEFAKVRCNGNDNTKCERCPPHTYTAIPNYSNGCHQ | |
| peptide | CRKCPTGSFDKVKCTGTQNSKCSCLPGWYCATDSSQTEDC | |
| IMP-266 | MKPLIMLICFAVILLQLGVTKVCQHNEVQLGNECCPPCGSGQRVTK | 570 |
| with native signal | VCTDYTSVTCTPCPNGTYVSGPYNCTDCTQCNVTQVMIRNCTSTNN | |
| peptide | TVCAPKNHTYFPTPGVQHHKQRQQNHTAHITVKQGKSGRHTLA | |
| IMP-267 | MDIKNLLTVCTIFYITTLATADIPTSSLPHAPVNGACDEGEYLDKR | 571 |
| with native signal | HNQCCNRCPPGEFAKVRCNGNDNTKCERCPPHTYTAIPNYSNGCHQ | |
| peptide | CRKCPTGSFDKVKCTGTQNSKCSCLPGWYCATDSSQTEDCRDCIPK | |
| | RKMSMRILWWNR | |
| IMP-268 | MMDINYRLAICTILYIGTIVATDIPTTPHSPINGSCDIGEYLDKKS | 572 |
| with native signal | GNCCKMCPPGAFAKVRCTSDNNTECANCPPGTFTSIPNYSNGCHQC | |
| peptide | RGHCPEGAFDEKRCTTTHDRICKCLPGWFCVTGSASLQCPMCIPKR | |
| | KCPCGYFGGIDELGNPLCKSCCKGEYCEHIRSYRPGYVPCNLSKCN | |
| IMP-269 | MDIKSLLAVCTILYITTLVTADIHTPLPPHASVNGSCAEGEYLDKT | 573 |
| with native signal | HNRCCNRCPPGEFAKVRCSGSYNTKCERCPHHTYTAIPNYSNGCHQ | |
| peptide | CRKCPKGSFDKVKCTGTQNSKCACLPGWYCATDSSQTEDCRDCVPK | |
| | RRCPCGYFGGIDKLGNPICKSCCVGKYCDDIRNHRVGPFPPCKLSK | |
| | CN | |
| IMP-270 | MDIKNLLTACTIFYITTLATADIPTSSLPHAPVNGACDEGEYLDKR | 574 |
| with native signal | HNQCCNQCPPGEFAKVRCNGNDNTKCERCPPHTYTAIPNYSNGCHQ | |
| peptide | CRKCPTGSFDKVKCTGTQNSKCSCLPGWYCATDSSQTEDCRDCIPK | |
| | RRCPCGYFGGIDEQGNPICKSCCVGEYCDYLRNYRLDPFPPCKLSK | |
| | CN | |
| IMP-271 | MDIKNLLTVCTIFYITTLATADIPTPPLPHAPVNGACDEGEYLDKR | 575 |
| with native signal | HNQCCNQCPPGEFAKVRCNGNDNTKCERCPPHTYTAIPNYSNGCHQ | |
| peptide | CRKCPTGSFDKVKCTGTQNSKCSCLPGWYCATDSSQTEDCRDCIPK | |
| | RRCPCGYFGGIDEQGNPICKSCCVGEYCDYLRNYRLDPFPPCKLSK | |
| | CN | |
| IMP-272 | MKSYILLLLLSYIIIINSDITPHEPSNGKCKDNEYKHHHLCCLSCP | 576 |
| with native signal | PGTYASRLCDSKTNTNTQCTPCGSGTFTSRNNHLPACLSCNGRCDS | |
| peptide | NQVETRSCNTTHNRICDCAPGYYCLLKGSSGCKACVSQTKCGIGYG | |
| | VSGHTPTGDVVCSPCGLGTYSHTVSSVDKCEPVPSNTFNYIDVEIN | |
| | LYPVNDTSCTRTTTTGLSESISTSELTITMNHKDCDPVFRDGYFSV | |
| | LNKVATSGFFTGQNRYQNISKVCTLNFEIKCNNKDSYSSSKQLTKA | |
| | KNDDDSIMPHSETVTLVGDCLSSVDIYILYSNTNTQDYETDTISYH | |
| | VGNVLDVDSHMPGSCDIHKLITNSSHSLL | |

In some aspects and embodiments, the CD30L binding protein comprises an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 5. In some embodiments, the CD30L binding protein comprises an amino acid sequence at least about 85% identical to the amino acid sequence of a protein set forth in Table 5. In some embodiments, the CD30L binding protein comprises an amino acid sequence at least about 90% identical to the amino acid sequence of a protein set forth in Table 5. In some embodiments, the CD30L binding protein comprises an amino acid sequence at least about 95% identical to the amino acid sequence of a protein set forth in Table 5. In some embodiments, the CD30L binding protein comprises an amino acid sequence at least about 99% identical to the amino acid sequence of a protein set forth in Table 5.

In some embodiments, the CD30L binding protein comprises an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 5. In some embodiments, the CD30L binding protein comprises an amino acid sequence about 85% identical to the amino acid sequence of a protein set forth in Table 5. In some embodiments, the CD30L binding protein comprises an amino acid sequence about 90% identical to the amino acid sequence of a protein set forth in Table 5. In some embodiments, the CD30L binding protein comprises an amino acid sequence about 95% identical to the amino acid sequence of a protein set forth in Table 5. In some embodiments, the CD30L binding protein comprises an amino acid sequence about 99% identical to the amino acid sequence of a protein set forth in Table 5. In some embodiments, the CD30L binding protein comprises an amino acid sequence about 100% identical to the amino acid sequence of a protein set forth in Table 5.

In some embodiments, the CD30L binding protein consists of an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 5. In some embodiments, the CD30L binding protein consists of an amino acid sequence at least about 85% identical to the amino acid sequence of a protein set forth in Table 5. In some embodiments, the CD30L binding protein consists of an amino acid sequence at least about 90% identical to the amino acid sequence of a protein set forth in Table 5. In some embodiments, the CD30L binding protein consists of an amino acid sequence at least about 95% identical to the amino acid sequence of a protein set forth in Table 5. In some embodiments, the CD30L binding protein consists of an amino acid sequence at least about 99% identical to the amino acid sequence of a protein set forth in Table 5.

In some embodiments, the CD30L binding protein consists of an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 5. In some embodiments, the CD30L binding protein consists of an amino acid sequence about 85% identical to the amino acid sequence of a protein set forth in Table 5. In some embodiments, the CD30L binding protein consists of an amino acid sequence about 90% identical to the amino acid sequence of a protein set forth in Table 5. In some embodiments, the CD30L binding protein consists of an amino acid sequence about 95% identical to the amino acid sequence of a protein set forth in Table 5. In some embodiments, the CD30L binding protein consists of an amino acid sequence about 99% identical to the amino acid sequence of a protein set forth in Table 5. In some embodiments, the CD30L binding protein consists of an amino acid sequence about 100% identical to the amino acid sequence of a protein set forth in Table 5.

In some embodiments, the CD30L binding protein comprises the amino acid sequence of a protein set forth in Table 5, and further comprising 1 or more but less than 15% (less than 12%, less than 10%, less than 8%), amino acid variations (e.g., substitutions, additions, deletions, etc.). In some embodiments, the CD30L binding protein comprises the amino acid sequence of a protein set forth in Table 5, and further comprising at least about 1, 2, 3, 4, 5, 6, 7, 8, 9, or 10 amino acid variations (e.g., substitutions, additions, deletions, etc.). In some embodiments, the CD30L binding protein comprises the amino acid sequence of a protein set forth in Table 5, and further consisting of at least about 1, 2, 3, 4, 5, 6, 7, 8, 9, or 10 amino acid variations (e.g., substitutions, additions, deletions, etc.). In some embodiments, the CD30L binding protein comprises the amino acid sequence of a protein set forth in Table 5, and further comprising about 1, 2, 3, 4, 5, 6, 7, 8, 9, or 10 amino acid variations (e.g., substitutions, additions, deletions, etc.). In some embodiments, the CD30L binding protein comprises the amino acid sequence of a protein set forth in Table 5, and further consisting of about 1, 2, 3, 4, 5, 6, 7, 8, 9, or 10 amino acid variations (e.g., substitutions, additions, deletions, etc.). In some embodiments, the CD30L binding protein comprises the amino acid sequence of a protein set forth in Table 5, and further comprising or no more than about 1, 2, 3, 4, 5, 6, 7, 8, 9, or 10 amino acid variations (e.g., substitutions, additions, deletions, etc.).

In some embodiments, the CD30L binding protein consists of the amino acid sequence of a protein set forth in Table 5, and further comprising 1 or more but less than 15% (less than 12%, less than 10%, less than 8%), amino acid variations (e.g., substitutions, additions, deletions, etc.). In some embodiments, the CD30L binding protein consists of the amino acid sequence of a protein set forth in Table 5, and further comprising at least about 1, 2, 3, 4, 5, 6, 7, 8, 9, or 10 amino acid variations (e.g., substitutions, additions, deletions, etc.). In some embodiments, the CD30L binding protein consists of the amino acid sequence of a protein set forth in Table 5, and further consisting of at least about 1, 2, 3, 4, 5, 6, 7, 8, 9, or 10 amino acid variations (e.g., substitutions, additions, deletions, etc.). In some embodiments, the CD30L binding protein consists of the amino acid sequence of a protein set forth in Table 5, and further comprising about 1, 2, 3, 4, 5, 6, 7, 8, 9, or 10 amino acid variations (e.g., substitutions, additions, deletions, etc.). In some embodiments, the CD30L binding protein consists of the amino acid sequence of a protein set forth in Table 5, and further consisting of about 1, 2, 3, 4, 5, 6, 7, 8, 9, or 10 amino acid variations (e.g., substitutions, additions, deletions, etc.). In some embodiments, the CD30L binding protein consists of the amino acid sequence of a protein set forth in Table 5, and further comprising or no more than about 1, 2, 3, 4, 5, 6, 7, 8, 9, or 10 amino acid variations (e.g., substitutions, additions, deletions, etc.).

In some embodiments, the CD30L binding protein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, the CD30L binding protein comprises an amino acid sequence at least 85% identical to the amino acid sequence set forth in any one of SEQ ID NOS: 465-576. In some embodiments, the CD30L binding protein comprises an amino acid sequence at least 90% identical to the amino acid sequence set forth in any one of SEQ ID NOS: 465-576. In some embodiments, the CD30L binding protein comprises an amino acid sequence at least 95% identical to the amino acid sequence set forth in any one of SEQ ID NOS: 465-576. In some embodiments, the CD30L binding protein comprises an amino acid sequence at least 99% identical to the amino acid sequence set forth in any one of SEQ ID NOS: 465-576.

In some embodiments, the CD30L binding protein comprises an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, the CD30L binding protein comprises an amino acid sequence 85% identical to the amino acid sequence set forth in any one of SEQ ID NOS: 465-576. In some embodiments, the CD30L binding protein comprises an amino acid sequence 90% identical to the amino acid sequence set forth in any one of SEQ ID NOS: 465-576. In some embodiments, the CD30L binding protein comprises an amino acid sequence 95% identical to the amino acid sequence set forth in any one of SEQ ID NOS: 465-576. In some embodiments, the CD30L binding protein comprises an amino acid sequence 99% identical to the amino acid sequence set forth in any one of SEQ ID NOS: 465-576. In some embodiments, the CD30L binding protein comprises an amino acid sequence 100% identical to the amino acid sequence set forth in any one of SEQ ID NOS: 465-576.

In some embodiments, the CD30L binding protein consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, the CD30L binding protein consists of an amino acid sequence at least 85% identical to the amino acid sequence set forth in any one of SEQ ID NOS: 465-576. In some embodiments, the CD30L binding protein consists of an amino acid sequence at least 90% identical to the amino acid sequence set forth in any one of SEQ ID NOS: 465-576. In some embodiments, the CD30L binding protein consists of an amino acid sequence at least 95% identical to the amino acid sequence set forth in any one of SEQ ID NOS: 465-576. In some embodiments, the CD30L binding protein consists of an amino acid sequence at least 99% identical to the amino acid sequence set forth in any one of SEQ ID NOS: 465-576.

In some embodiments, the CD30L binding protein consists of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, the CD30L binding protein consists of an amino acid sequence 85% identical to the amino acid sequence set forth in any one of SEQ ID NOS: 465-576. In some embodiments, the CD30L binding protein consists of an amino acid sequence 90% identical to the amino acid sequence set forth in any one of SEQ ID NOS: 465-576. In some embodiments, the CD30L binding protein consists of an amino acid sequence 95% identical to the amino acid sequence set forth in any one of SEQ ID NOS: 465-576. In some embodiments, the CD30L binding protein consists of an amino acid sequence 99% identical to the amino acid sequence set forth in any one of SEQ ID NOS: 465-576. In some embodiments, the CD30L binding protein consists of an amino acid sequence 100% identical to the amino acid sequence set forth in any one of SEQ ID NOS: 465-576.

In some embodiments, the CD30L binding protein comprises the amino acid sequence set forth in any one of SEQ ID NOS: 465-576, and further comprising 1 or more but less than 15% (less than 12%, less than 10%, less than 8%), amino acid variations (e.g., substitutions, additions, deletions, etc.). In some embodiments, the CD30L binding protein comprises the amino acid sequence set forth in any one of SEQ ID NOS: 465-576, and further comprising at least about 1, 2, 3, 4, 5, 6, 7, 8, 9, or 10 amino acid variations (e.g., substitutions, additions, deletions, etc.). In some embodiments, the CD30L binding protein comprises the amino acid sequence set forth in any one of SEQ ID NOS: 465-576, and further comprising about 1, 2, 3, 4, 5, 6, 7, 8, 9, or 10 amino acid variations (e.g., substitutions, additions, deletions, etc.). In some embodiments, the CD30L binding protein comprises the amino acid sequence set forth in any one of SEQ ID NOS: 465-576, and further consisting of about 1, 2, 3, 4, 5, 6, 7, 8, 9, or 10 amino acid variations (e.g., substitutions, additions, deletions, etc.). In some embodiments, the CD30L binding protein comprises the amino acid sequence set forth in any one of SEQ ID NOS: 465-576, and further comprising or consists of no more than about 1, 2, 3, 4, 5, 6, 7, 8, 9, or 10 amino acid variations (e.g., substitutions, additions, deletions, etc.).

In some embodiments, the CD30L binding protein consists of the amino acid sequence set forth in any one of SEQ ID NOS: 465-576, and further comprising 1 or more but less than 15% (less than 12%, less than 10%, less than 8%), amino acid variations (e.g., substitutions, additions, deletions, etc.). In some embodiments, the CD30L binding protein consists of the amino acid sequence set forth in any one of SEQ ID NOS: 465-576, and further comprising at least about 1, 2, 3, 4, 5, 6, 7, 8, 9, or 10 amino acid variations (e.g., substitutions, additions, deletions, etc.). In some embodiments, the CD30L binding protein consists of the amino acid sequence set forth in any one of SEQ ID NOS: 465-576, and further comprising about 1, 2, 3, 4, 5, 6, 7, 8, 9, or 10 amino acid variations (e.g., substitutions, additions, deletions, etc.). In some embodiments, the CD30L binding protein consists of the amino acid sequence set forth in any one of SEQ ID NOS: 465-576, and further consisting of about 1, 2, 3, 4, 5, 6, 7, 8, 9, or 10 amino acid variations (e.g., substitutions, additions, deletions, etc.). In some embodiments, the CD30L binding protein consists of the amino acid sequence set forth in any one of SEQ ID NOS: 465-576, and further consisting of no more than about 1, 2, 3, 4, 5, 6, 7, 8, 9, or 10 amino acid variations (e.g., substitutions, additions, deletions, etc.).

In some aspects and embodiments, a fusion protein described herein comprises a protein (or a functional fragment, functional variant, or functional fragment/variant thereof) comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 5. In some embodiments, a fusion protein described herein comprises a protein (or a functional fragment, functional variant, or functional fragment/variant thereof) comprising an amino acid sequence at least about 85% identical to the amino acid sequence of a protein set forth in Table 5. In some embodiments, a fusion protein described herein comprises a protein (or a functional fragment, functional variant, or functional fragment/variant thereof) comprising an amino acid sequence at least about 90% identical to the amino acid sequence of a protein set forth in Table 5. In some embodiments, a fusion protein described herein comprises a protein (or a functional fragment, functional variant, or functional fragment/variant thereof) comprising an amino acid sequence at least about 95% identical to the amino acid sequence of a protein set forth in Table 5. In some embodiments, a fusion protein described herein comprises a protein (or a functional fragment, functional variant, or functional fragment/variant thereof) comprising an amino acid sequence at least about 99% identical to the amino acid sequence of a protein set forth in Table 5.

In some embodiments, a fusion protein described herein comprises a protein (or a functional fragment, functional variant, or functional fragment/variant thereof) comprising an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 5. In some embodiments, a fusion protein described herein comprises a protein (or a functional fragment, functional variant, or functional fragment/variant thereof) comprising an amino acid sequence about 85% identical to the amino acid sequence of a protein set forth in Table 5. In some embodiments, a fusion protein described herein comprises a protein ((or a functional fragment, functional variant, or functional fragment/variant thereof) comprising an amino acid sequence about 90% identical to the amino acid sequence of a protein set forth in Table 5. In some embodiments, a fusion protein described herein comprises a protein (or a functional fragment, functional variant, or functional fragment/variant thereof) comprising an amino acid sequence about 95% identical to the amino acid sequence of a protein set forth in Table 5. In some embodiments, a fusion protein described herein comprises a protein (or a functional fragment, functional variant, or functional fragment/variant thereof) comprising an amino acid sequence about 99% identical to the amino acid sequence of a protein set forth in Table 5. In some embodiments, a fusion protein described herein comprises a protein (or a functional fragment, functional variant, or functional fragment/variant thereof) comprising an amino acid sequence about 100% identical to the amino acid sequence of a protein set forth in Table 5.

In some embodiments, a fusion protein described herein comprises a protein (or a functional fragment, functional variant, or functional fragment/variant thereof) consisting of an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 5. In some embodiments, a fusion protein described herein comprises a protein (or a functional fragment, functional variant, or functional fragment/variant thereof) consisting of an amino acid sequence at least about 85% identical to the amino acid sequence of a protein set forth in Table 5. In some embodiments, a fusion protein described herein comprises a protein (or a functional fragment, functional variant, or functional fragment/variant thereof) consisting of an amino acid sequence at least about 90% identical to the amino acid sequence of a protein set forth in Table 5. In some embodiments, a fusion protein described herein comprises a protein (or a functional fragment, functional variant, or functional fragment/variant thereof) consisting of an amino acid sequence at least about 95% identical to the amino acid sequence of a protein set forth in Table 5. In some embodiments, a fusion protein described herein comprises a protein (or a functional fragment, functional variant, or functional fragment/variant thereof) consisting of an amino acid sequence at least about 99% identical to the amino acid sequence of a protein set forth in Table 5.

In some embodiments, a fusion protein described herein comprises a protein (or a functional fragment, functional variant, or functional fragment/variant thereof) consisting of an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 5. In some embodiments, a fusion protein described herein comprises a protein (or a functional fragment, functional variant, or functional fragment/variant thereof) consisting of an amino acid sequence about 85% identical to the amino acid sequence of a protein set forth in Table 5. In some embodiments, a fusion protein described herein comprises a protein (or a functional fragment, functional variant, or functional fragment/variant thereof) consisting of an amino acid sequence about 90% identical to the amino acid sequence of a protein set forth in Table 5. In some embodiments, a fusion protein described herein comprises a protein (or a functional fragment, functional variant, or functional fragment/variant thereof) consisting of an amino acid sequence about 95% identical to the amino acid sequence of a protein set forth in Table 5. In some embodiments, a fusion protein described herein comprises a protein (or a functional fragment, functional variant, or functional fragment/variant thereof) consisting of an amino acid sequence about 99% identical to the amino acid sequence of a protein set forth in Table 5. In some embodiments, a fusion protein described herein comprises a protein (or a functional fragment, functional variant, or functional fragment/variant thereof) consisting of an amino acid sequence about 100% identical to the amino acid sequence of a protein set forth in Table 5.

In some embodiments, a fusion protein described herein comprises a protein (or a functional fragment, functional variant, or functional fragment/variant thereof) comprising the amino acid sequence of a protein set forth in Table 5, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 5, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 5, and further consisting of at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 5, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 5, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 5, and further comprising or no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence of a protein set forth in Table 5, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 5, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 5, and further consisting of at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 5, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 5, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 5, and further comprising or no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence at least 85% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence at least 90% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence at least 95% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence at least 99% identical to the amino acid sequence set forth in any one of SEQ ID NOS: 465-576.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence 85% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence 90% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence 95% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence 99% identical to the amino acid sequence set forth in any one of SEQ ID NOS: 465-576. In some embodiments, a fusion protein described herein comprises a protein (or a functional fragment, functional variant, or functional fragment/variant thereof) comprising an amino acid sequence 100% identical to the amino acid sequence set forth in any one of SEQ ID NOS: 465-576.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence at least 85% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence at least 90% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence at least 95% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence at least 99% identical to the amino acid sequence set forth in any one of SEQ ID NOS: 465-576.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence 85% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence 90% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence 95% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence 99% identical to the amino acid sequence set forth in any one of SEQ ID NOS: 465-576. In some embodiments, a fusion protein described herein comprises a protein (or a functional fragment, functional variant, or functional fragment/variant thereof) consisting of an amino acid sequence 100% identical to the amino acid sequence set forth in any one of SEQ ID NOS: 465-576.

In some embodiments, a fusion protein described herein comprises a protein (or a functional fragment, functional variant, or functional fragment/variant thereof) comprising the amino acid sequence set forth in any one of SEQ ID NOS: 465-576, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 465-576, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 465-576, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 465-576, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 465-576, and further comprising or consists of no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence set forth in any one of SEQ ID NOS: 465-576, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 465-576, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 465-576, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 465-576, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 465-576, and further consisting of no more than about 1, 2, 3, 4, 5, 6, 7, 8, 9, or 10 amino acid variations (e.g., substitutions, additions, deletions, etc.).

### (i) Exemplary Properties of CD30L Binding Proteins

In some embodiments, the CD30L binding proteins described herein are immunosuppressive (e.g., when administered to a subject). In some embodiments, the CD30L binding proteins described herein are anti-inflammatory (e.g., when administered to a subject). In some embodiments, the CD30L binding proteins described herein suppress pro-inflammatory response (e.g., when administered to a subject).

In some embodiments, the CD30L binding protein is capable of specifically binding CD30L. In some embodiments, the CD30L binding protein is capable of inhibiting or reducing (e.g., preventing) binding of hCD30L to hCD30.

In some embodiments, the CD30L binding proteins described herein bind a subset (e.g., one or more) TNFSF ligand. In some embodiments, the CD30L binding protein specifically binds CD30L. In some embodiments, the CD30L binding protein specifically binds hCD30L.

In some embodiments, the CD30L binding protein specifically binds CD30L expressed on the surface of an immune cell. In some embodiments, the CD30L binding protein specifically binds CD30L expressed on the surface of an activated immune cell. In some embodiments, the CD30L binding protein specifically binds hCD30L expressed on the surface of an activated immune cell.

In some embodiments, the CD30L binding protein can act as a decoy receptor for a TNFSF ligand described herein (e.g., CD30L). In some embodiments, the CD30L binding protein inhibits or reduces (e.g., prevents) binding of CD30L to CD30. In some embodiments, the CD30L binding protein specifically binds to CD30L and inhibits binding of CD30L to CD30. In some embodiments, the CD30L binding protein inhibits or reduces (e.g., prevents) binding of hCD30L to hCD30. In some embodiments, the CD30L binding protein specifically binds to hCD30 and inhibits or reduces (e.g., prevents) binding of hCD30 to hCD30L.

### 5.2.2 Integrin Targeting Immunomodulatory Fusion Proteins

The present disclosure provides, inter alia, fusion proteins (e.g., immunomodulatory fusion proteins (JFPs)) (and functional fragments and variants thereof) that e.g., comprise one or more integrin binding domain (e.g., described herein) and one or more immunomodulatory protein (e.g., described herein). Integrin binding domains include, those, e.g., described herein, see, e.g., § 5.2.2.1. Immunomodulatory proteins described herein include, e.g., those described in §§ 5.2.1.1, 5.2.1.2, 5.2.1.3, and 5.2.1.4. Immunomodulatory proteins described herein include, e.g., those that specifically bind to IL-10 (e.g., human IL-10)) (see, e.g., § 5.2.1.1), those that specifically bind to TL1A (e.g., human TL1A)) (see, e.g., § 5.2.1.2), those that specifically bind to TNFα (e.g., human TNFα)) (see, e.g., § 5.2.1.3), and those that specifically bind to CD30L (e.g., human CD30L)) (see, e.g., § 5.2.1.4).

The amino acid sequence of various reference human proteins referred to throughout the instant disclosure is set forth Table 6 below.

TABLE 6
The Amino Acid Sequence of Human Reference Proteins.

| | | | SEQ |
|---|---|---|---|
| Description | | Amino Acid Sequence | ID NO |
| Human | α4 | MAWEARREPGPRRAAVRETVMLLLCLGVPTGRPYNVDTESALLY | 577 |
| α4β7 | Immature - | QGPHNTLFGYSVVLHSHGANRWLLVGAPTANWLANASVINPGAI | |
| Integrin | Signal | YRCRIGKNPGQTCEQLQLGSPNGEPCGKTCLEERDNQWLGVTLS | |
| | Peptide | RQPGENGSIVTCGHRWKNIFYIKNENKLPTGGCYGVPPDLRTEL | |
| | Underlined | SKRIAPCYQDYVKKFGENFASCQAGISSFYTKDLIVMGAPGSSY | |
| | UniProt | WTGSLFVYNITTNKYKAFLDKQNQVKFGSYLGYSVGAGHFRSQH | |
| | ID: P13612 | TTEVVGGAPQHEQIGKAYIFSIDEKELNILHEMKGKKLGSYFGA | |
| | | SVCAVDLNADGFSDLLVGAPMQSTIREEGRVFVYINSGSGAVMN | |
| | | AMETNLVGSDKYAARFGESIVNLGDIDNDGFEDVAIGAPQEDDL | |
| | | QGAIYIYNGRADGISSTFSQRIEGLQISKSLSMFGQSISGQIDA | |
| | | DNNGYVDVAVGAFRSDSAVLLRTRPVVIVDASLSHPESVNRTKF | |
| | | DCVENGWPSVCIDLTLCFSYKGKEVPGYIVLFYNMSLDVNRKAE | |
| | | SPPRFYFSSNGTSDVITGSIQVSSREANCRTHQAFMRKDVRDIL | |
| | | TPIQIEAAYHLGPHVISKRSTEEFPPLQPILQQKKEKDIMKKTI | |
| | | NFARFCAHENCSADLQVSAKIGFLKPHENKTYLAVGSMKTLMLN | |
| | | VSLFNAGDDAYETTLHVKLPVGLYFIKILELEEKQINCEVTDNS | |
| | | GVVQLDCSIGYIYVDHLSRIDISFLLDVSSLSRAEEDLSITVHA | |
| | | TCENEEEMDNLKHSRVTVAIPLKYEVKLTVHGFVNPTSFVYGSN | |
| | | DENEPETCMVEKMNLTFHVINTGNSMAPNVSVEIMVPNSFSPQT | |
| | | DKLFNILDVQTTTGECHFENYQRVCALEQQKSAMQTLKGIVRFL | |
| | | SKTDKRLLYCIKADPHCLNFLCNFGKMESGKEASVHIQLEGRPS | |
| | | ILEMDETSALKFEIRATGFPEPNPRVIELNKDENVAHVLLEGLH | |
| | | HQRPKRYFTIVIISSSLLLGLIVLLLISYVMWKAGFFKRQYKSI | |
| | | LQEENRRDSWSYINSKSNDD | |
| | α4 | YNVDTESALLYQGPHNTLFGYSVVLHSHGANRWLLVGAPTANWL | 578 |
| | Mature - | ANASVINPGAIYRCRIGKNPGQTCEQLQLGSPNGEPCGKTCLEE | |
| | No Signal | RDNQWLGVTLSRQPGENGSIVTCGHRWKNIFYIKNENKLPTGGC | |
| | Peptide | YGVPPDLRTELSKRIAPCYQDYVKKFGENFASCQAGISSFYTKD | |
| | UniProt | LIVMGAPGSSYWTGSLFVYNITTNKYKAFLDKQNQVKFGSYLGY | |
| | ID: | SVGAGHFRSQHTTEVVGGAPQHEQIGKAYIFSIDEKELNILHEM | |
| | P13612 | KGKKLGSYFGASVCAVDLNADGFSDLLVGAPMQSTIREEGRVFV | |
| | | YINSGSGAVMNAMETNLVGSDKYAARFGESIVNLGDIDNDGFED | |
| | | VAIGAPQEDDLQGAIYIYNGRADGISSTFSQRIEGLQISKSLSM | |
| | | FGQSISGQIDADNNGYVDVAVGAFRSDSAVLLRTRPVVIVDASL | |
| | | SHPESVNRTKFDCVENGWPSVCIDLTLCFSYKGKEVPGYIVLFY | |
| | | NMSLDVNRKAESPPRFYFSSNGTSDVITGSIQVSSREANCRTHQ | |
| | | AFMRKDVRDILTPIQIEAAYHLGPHVISKRSTEEFPPLQPILQQ | |
| | | KKEKDIMKKTINFARFCAHENCSADLQVSAKIGFLKPHENKTYL | |
| | | AVGSMKTLMLNVSLFNAGDDAYETTLHVKLPVGLYFIKILELEE | |
| | | KQINCEVTDNSGVVQLDCSIGYIYVDHLSRIDISFLLDVSSLSR | |
| | | AEEDLSITVHATCENEEEMDNLKHSRVTVAIPLKYEVKLTVHGF | |
| | | VNPTSFVYGSNDENEPETCMVEKMNLTFHVINTGNSMAPNVSVE | |
| | | IMVPNSFSPQTDKLFNILDVQTTTGECHFENYQRVCALEQQKSA | |
| | | MQTLKGIVRFLSKTDKRLLYCIKADPHCLNFLCNFGKMESGKEA | |
| | | SVHIQLEGRPSILEMDETSALKFEIRATGFPEPNPRVIELNKDE | |
| | | NVAHVLLEGLHHQRPKRYFTIVIISSSLLLGLIVLLLISYVMWK | |
| | | AGFFKRQYKSILQEENRRDSWSYINSKSNDD | |
| | β7 | MVALPMVLVLLLVLSRGESELDAKIPSTGDATEWRNPHLSMLGS | 579 |
| | Immature - | CQPAPSCQKCILSHPSCAWCKQLNFTASGEAEARRCARREELLA | |
| | Signal | RGCPLEELEEPRGQQEVLQDQPLSQGARGEGATQLAPQRVRVTL | |
| | Peptide | RPGEPQQLQVRFLRAEGYPVDLYYLMDLSYSMKDDLERVRQLGH | |
| | Underlined | ALLVRLQEVTHSVRIGFGSFVDKTVLPFVSTVPSKLRHPCPTRL | |
| | UniProt | ERCQSPFSFHHVLSLTGDAQAFEREVGRQSVSGNLDSPEGGFDA | |
| | ID: P26010 | ILQAALCQEQIGWRNVSRLLVFTSDDTFHTAGDGKLGGIFMPSD | |
| | | GHCHLDSNGLYSRSTEFDYPSVGQVAQALSAANIQPIFAVTSAA | |
| | | LPVYQELSKLIPKSAVGELSEDSSNVVQLIMDAYNSLSSTVTLE | |
| | | HSSLPPGVHISYESQCEGPEKREGKAEDRGQCNHVRINQTVTFW | |
| | | VSLQATHCLPEPHLLRLRALGFSEELIVELHTLCDCNCSDTQPQ | |
| | | APHCSDGQGHLQCGVCSCAPGRLGRLCECSVAELSSPDLESGCR | |
| | | APNGTGPLCSGKGHCQCGRCSCSGQSSGHLCECDDASCERHEGI | |
| | | LCGGFGRCQCGVCHCHANRTGRACECSGDMDSCISPEGGLCSGH | |
| | | GRCKCNRCQCLDGYYGALCDQCPGCKTPCERHRDCAECGAFRTG | |
| | | PLATNCSTACAHTNVTLALAPILDDGWCKERTLDNQLFFELVED | |
| | | DARGTVVLRVRPQEKGADHTQAIVLGCVGGIVAVGLGLVLAYRL | |
| | | SVEIYDRREYSRFEKEQQQLNWKQDSNPLYKSAITTTINPRFQE | |
| | | ADSPTL | |
| | β7 | ELDAKIPSTGDATEWRNPHLSMLGSCQPAPSCQKCILSHPSCAW | 580 |
| | Mature - | CKQLNFTASGEAEARRCARREELLARGCPLEELEEPRGQQEVLQ | |
| | No Signal | DQPLSQGARGEGATQLAPQRVRVTLRPGEPQQLQVRFLRAEGYP | |
| | Peptide | VDLYYLMDLSYSMKDDLERVRQLGHALLVRLQEVTHSVRIGFGS | |
| | UniProt | FVDKTVLPFVSTVPSKLRHPCPTRLERCQSPFSFHHVLSLTGDA | |
| | ID: P26010 | QAFEREVGRQSVSGNLDSPEGGFDAILQAALCQEQIGWRNVSRL | |
| | | LVFTSDDTFHTAGDGKLGGIFMPSDGHCHLDSNGLYSRSTEFDY | |
| | | PSVGQVAQALSAANIQPIFAVTSAALPVYQELSKLIPKSAVGEL | |
| | | SEDSSNVVQLIMDAYNSLSSTVTLEHSSLPPGVHISYESQCEGP | |
| | | EKREGKAEDRGQCNHVRINQTVTFWVSLQATHCLPEPHLLRLRA | |
| | | LGFSEELIVELHTLCDCNCSDTQPQAPHCSDGQGHLQCGVCSCA | |
| | | PGRLGRLCECSVAELSSPDLESGCRAPNGTGPLCSGKGHCQCGR | |
| | | CSCSGQSSGHLCECDDASCERHEGILCGGFGRCQCGVCHCHANR | |
| | | TGRACECSGDMDSCISPEGGLCSGHGRCKCNRCQCLDGYYGALC | |
| | | DQCPGCKTPCERHRDCAECGAFRTGPLATNCSTACAHTNVTLAL | |
| | | APILDDGWCKERTLDNQLFFELVEDDARGTVVLRVRPQEKGADH | |
| | | TQAIVLGCVGGIVAVGLGLVLAYRLSVEIYDRREYSRFEKEQQQ | |
| | | LNWKQDSNPLYKSAITTTINPRFQEADSPTL | |

| | | |
|---|---|---|
| hMADCAM-1 | MDFGLALLLAGLIGLLLGQSLQVKPLQVEPPEPVVAVALGASRQ | 581 |
| (Immature - Signal | LTCRLACADRGASVQWRGLDTSLGAVQSDTGRSVLTVRNASLSA | |
| Peptide Underlined) | AGTRVCVGSCGGRTFQHTVQLLVYAFPDQLTVSPAALVPGDPEV | |
| UniProt ID: Q13477 | ACTAHKVTPVDPNALSFSLLVGGQELEGAQALGPEVQEEEEEPQ | |
| | GDEDVLFRVTERWRLPPLGTPVPPALYCQATMRLPGLELSHRQA | |
| | IPVLHSPTSPEPPDTTSPESPDTTSPESPDTTSQEPPDTTSPEP | |
| | PDKTSPEPAPQQGSTHTPRSPGSTRTRRPEISQAGPTQGEVIPT | |
| | GSSKPAGDQLPAALWTSSAVLGLLLLALPTYHLWKRCRHLAEDD | |
| | THPPASLRLLPQVSAWAGLRGTGQVGISPS | |
| hMADCAM-1 | QSLQVKPLQVEPPEPVVAVALGASRQLTCRLACADRGASVQWRG | 582 |
| (Mature - No Signal | LDTSLGAVQSDTGRSVLTVRNASLSAAGTRVCVGSCGGRTFQHT | |
| Peptide) | VQLLVYAFPDQLTVSPAALVPGDPEVACTAHKVTPVDPNALSFS | |
| UniProt ID: Q13477 | LLVGGQELEGAQALGPEVQEEEEEPQGDEDVLFRVTERWRLPPL | |
| | GTPVPPALYCQATMRLPGLELSHRQAIPVLHSPTSPEPPDTTSP | |
| | ESPDTTSPESPDTTSQEPPDTTSPEPPDKTSPEPAPQQGSTHTP | |
| | RSPGSTRTRRPEISQAGPTQGEVIPTGSSKPAGDQLPAALWTSS | |
| | AVLGLLLLALPTYHLWKRCRHLAEDDTHPPASLRLLPQVSAWAG | |
| | LRGTGQVGISPS | |

### 5.2.2.1 Integrin Binding Domains

As described above, fusion proteins described herein comprise one or more integrin binding domain.

Integrins are a family of ubiquitous αβ heterodimeric receptors that exist in multiple conformations and interact with a diverse group of ligands. These molecules, inter alia, mediate interactions between cells and cells with the extracellular matrix (ECM) and thereby, e.g., serve a role in signaling and homeostasis. By facilitating dynamic linkages between the intracellular actin cytoskeleton and the ECM, integrins also transduce both external and internal mechanochemical cues and bi-directional signaling across the plasma membrane. In mammals, the family of integrins is comprised of about 24 αβ pairs of heterodimeric transmembrane adhesion receptors and cell-surface proteins. These pairings are known to involve about 18 a and about 8 β subunits. The ap pairings of integrin subunits dictate the specificity of the integrin to a particular ligand, modulate formation of intracellular adhesion complexes, and regulate downstream signaling. See, e.g., Mezu-Ndubuisi O J, Maheshwari A. The role of integrins in inflammation and angiogenesis. Pediatr Res. 2021 May; 89(7):1619-1626. doi: 10.1038/s41390-020-01177-9. Epub 2020 Oct. 7. PMID: 33027803; PMCID: PMC8249239; Alberts B, Johnson A, Lewis J, et al. Molecular Biology of the Cell. 4th edition. New York: Garland Science; 2002. Integrins. Available from: https://www.ncbi.nlm.nih.gov/books/NBK26867/; Danen E H J. Integrins: An Overview of Structural and Functional Aspects. In: Madame Curie Bioscience Database [Internet]. Austin (TX): Landes Bioscience; 2000-2013. Available from: https://www.ncbi.nlm.nih.gov/books/NBK6259/; the entire contents of each of which are incorporated herein by reference for all purposes.

In some embodiments, the integrin binding domain specifically binds a plurality of integrins. In some embodiments, the integrin binding domain selectively binds one or more integrin but not others. In some embodiments, the integrin binding domain specifically binds one or more integrin expressed on surface of one or more immune cell (e.g., T cells).

In some embodiments, the integrin binding domain specifically binds any one or more of all, α2β1, α10β1, α11β1, α5β1, α8β1, α5β1, α5β3, α5β5, α5β6, α5β8, αIIbβ3, α3β1, α6β1, α7β1, α6β4, α4β1, α9β1, α4β7, αEβ7, αLβ2, αMβ2, α×β2, and/or αDβ2. In some embodiments, the integrin binding domain specifically binds any one or more of α1β1, α2β1, α10β1, and/or α11β1. In some embodiments, the integrin binding domain specifically binds any one or more of α5β1, α8β1, α5β1, α5β3, α5β5, α5β6, α5β8, and/or αIIbβ3. In some embodiments, the integrin binding domain specifically binds any one or more of α3β1, α6β1, α7β31, and/or α6β4. In some embodiments, the integrin binding domain specifically binds any one or more of α4β1 and/or α9β1. In some embodiments, the integrin binding domain specifically binds any one or more of α4β7, αEβ7, αLβ2, αMβ2, αXβ2, and/or αDβ2.

In some preferred embodiments, the integrin binding domain specifically α4β7 integrin.

In some embodiments, the integrin binding domain comprises an antibody. Exemplary antibodies include, full-length antibodies, scFvs, Fabs, single domain antibodies (e.g., VHH), scFv-Fc, Fab-Fc, and single domain antibody-Fc (e.g., VHH-Fc).

In some embodiments, the integrin binding domain comprises a scFv. In some embodiments, the integrin binding domain comprises a Fab. In some embodiments, the integrin binding domain comprises a full-length antibody. In some embodiments, the integrin binding domain comprises a single domain antibody. In some embodiments, the integrin binding domain comprises a VHH. In some embodiments, the integrin binding domain comprises a scFv-Fc. In some embodiments, the integrin binding domain comprises a Fab-Fc. In some embodiments, the integrin binding domain comprises a (scFv)2-Fc. In some embodiments, the integrin binding domain comprises a (Fab)2-Fc.

In some embodiments, the integrin binding domain comprises an antibody mimetic (e.g., described herein). For example, a 10th type III domain of fibronectin (e.g., AdNectins®) or designed ankyrin repeat proteins (e.g., DARPins®).

Exemplary antibody-based integrin binding domains that specifically bind α4β7 integrin include those described in, e.g., U.S. Pat. Nos. 100,048,081B2, 109,187,161B2, 118,847,311B2, and 96,635,791B2, the entire contents of each of which is incorporated herein by reference for all purposes.

The amino acid sequence of exemplary α4β7 integrin binding domains that can be utilized in the fusion proteins described herein is provided in Table 2. The CDRs set forth in Table 7, are denoted according to Kabat. A person of ordinary skill in the art would be able to determine the CDRs as defined by another scheme, e.g., Chothia, IMGT, using ordinary methods known in the art.

TABLE 7
The Amino Acid Sequence of Exemplary α487 Integrin Binding Domains.

| Description | | SEQ ID NO | Amino Acid Sequence |
|---|---|---|---|
| Ab-1 | CDR-H1 | 583 | SYWMH |
| | CDR-H2 | 584 | EIDPSESNTNYNQKFKG |
| | CDR-H3 | 585 | GGYDGWDYAIDY |
| | CDR-L1 | 586 | RSSQSLAKSYGNTYLS |
| | CDR-L2 | 587 | GISNRFS |
| | CDR-L3 | 588 | LQGTHQPYT |
| | VH | 589 | QVQLVQSGAEVKKPGASVKVSCKGSGYTFTSYWMH |
| | | | WVRQAPGQRLEWIGEIDPSESNTNYNQKFKGRVTL |
| | | | TVDISASTAYMELSSLRSEDTAVYYCARGGYDGWD |
| | | | YAIDYWGQGTLVTVSS |
| | VL | 590 | DVVMTQSPLSLPVTPGEPASISCRSSQSLAKSYGN |
| | | | TYLSWYLQKPGQSPQLLIYGISNRFSGVPDRFSGS |
| | | | GSGTDFTLKISRVEAEDVGVYYCLQGTHQPYTFGQ |
| | | | GTKVEIKRTV |
| | HC | 591 | QVQLVQSGAEVKKPGASVKVSCKGSGYTFTSYWMH |
| | | | WVRQAPGQRLEWIGEIDPSESNTNYNQKFKGRVTL |
| | | | TVDISASTAYMELSSLRSEDTAVYYCARGGYDGWD |
| | | | YAIDYWGQGTLVTVSSASTKGPSVFPLAPSSKSTS |
| | | | GGTAALGCLVKDYFPEPVTVSWNSGALTSGVHTFP |
| | | | AVLQSSGLYSLSSVVTVPSSSLGTQTYICNVNHKP |
| | | | SNTKVDKKVEPKSCDKTHTCPPCPAPELAGAPSVF |
| | | | LFPPKPKDTLMISRTPEVTCVVVDVSHEDPEVKFN |
| | | | WYVDGVEVHNAKTKPREEQYNSTYRVVSVLTVLHQ |
| | | | DWLNGKEYKCKVSNKALPAPIEKTISKAKGQPREP |
| | | | QVYTLPPSRDELTKNQVSLTCLVKGFYPSDIAVEW |
| | | | ESNGQPENNYKTTPPVLDSDGSFFLYSKLTVDKSR |
| | | | WQQGNVFSCSVMHEALHNHYTQKSLSLSPGK |
| | LC | 592 | DVVMTQSPLSLPVTPGEPASISCRSSQSLAKSYGN |
| | | | TYLSWYLQKPGQSPQLLIYGISNRFSGVPDRFSGS |
| | | | GSGTDFTLKISRVEAEDVGVYYCLQGTHQPYTFGQ |
| | | | GTKVEIKRTVAAPSVFIFPPSDEQLKSGTASVVCL |
| | | | LNNFYPREAKVQWKVDNALQSGNSQESVTEQDSKD |
| | | | STYSLSSTLTLSKADYEKHKVYACEVTHQGLSSPV |
| | | | TKSFNRGEC |

In some embodiments, the integrin (e.g., α4β7 integrin) binding domain comprises an integrin (e.g., α4β7 integrin) binding domain provided in Table 7. In some embodiments, the integrin (e.g., α4β7 integrin) binding domain comprises a VH that comprises: a CDR-H1, a CDR-H2, and a CDR-H3; and VL comprises CDR-L1, a CDR-L2, and a CDR-L3. In some embodiments, the integrin (e.g., α4β7 integrin) binding domain comprises a VH that comprises: a CDR-H1, a CDR-H2, and a CDR-H3 set forth in Table 7; and VL comprises CDR-L1, a CDR-L2, and a CDR-L3 set forth in Table 7.

In some embodiments, the amino acid sequence of CDR-H1 comprises or consists of the amino acid sequence of a CDR-H1 of a VH set forth in Table 7, or the amino acid sequence of a CDR-H1 of a VH set forth in Table 7 comprising or consisting of 1, 2, or 3 amino acid variations (e.g., substitution, deletion, addition, etc.); the amino acid sequence of CDR-H2 comprises or consists of the amino acid sequence of a CDR-H2 of a VH set forth in Table 7, or the amino acid sequence of a CDR-H2 of a VH set forth in Table 7 comprising or consisting of 1, 2, or 3 amino acid variations (e.g., substitution, deletion, addition, etc.); the amino acid sequence of CDR-H3 comprises or consists of the amino acid sequence of a CDR-H3 of a VH set forth in Table 7, or the amino acid sequence of a CDR-H3 of a VH set forth in Table 7 comprising or consisting of 1, 2, or 3 amino acid variations (e.g., substitution, deletion, addition, etc.); the amino acid sequence of CDR-L1 comprises or consists of the amino acid sequence of a CDR-L1 of a VL set forth in Table 7, or the amino acid sequence of a CDR-L1 of a VL set forth in Table 7 comprising or consisting of 1, 2, or 3 amino acid variations (e.g., substitution, deletion, addition, etc.); the amino acid sequence of CDR-L2 comprises or consists of the amino acid sequence of a CDR-L2 of a VL set forth in Table 7, or the amino acid sequence of a CDR-L2 of a VL set forth in Table 7 comprising or consisting of 1, 2, or 3 amino acid variations (e.g., substitution, deletion, addition, etc.); and the amino acid sequence of CDR-L3 comprises or consists of the amino acid sequence of a CDR-L3 of a VL set forth in Table 7, or the amino acid sequence of a CDR-L3 of a VL set forth in Table 7 comprising or consisting of 1, 2, or 3 amino acid variations (e.g., substitution, deletion, addition, etc.).

In some embodiments, the amino acid sequence of CDR-H1 comprises or consists of the amino acid sequence of a CDR-H1 set forth in Table 7, or the amino acid sequence of a CDR-H1 set forth in Table 7 comprising or consisting of 1, 2, or 3 amino acid variations (e.g., substitution, deletion, addition, etc.); the amino acid sequence of CDR-H2 comprises or consists of the amino acid sequence of a CDR-H2 set forth in Table 7, or the amino acid sequence of a CDR-H2 set forth in Table 7 comprising or consisting of 1, 2, or 3 amino acid variations (e.g., substitution, deletion, addition, etc.); the amino acid sequence of CDR-H3 comprises or consists of the amino acid sequence of a CDR-H3 set forth in Table 7, or the amino acid sequence of a CDR-H3 set forth in Table 7 comprising or consisting of 1, 2, or 3 amino acid variations (e.g., substitution, deletion, addition, etc.); the amino acid sequence of CDR-L1 comprises or consists of the amino acid sequence of a CDR-L1 set forth in Table 7, or the amino acid sequence CDR1 of a VL set forth in Table 7 comprising or consisting of 1, 2, or 3 amino acid variations (e.g., substitution, deletion, addition, etc.); the amino acid sequence of CDR-L2 comprises or consists of the amino acid sequence of a CDR-L2 set forth in Table 7, or the amino acid sequence of a CDR-L2 set forth in Table 7 comprising or consisting of 1, 2, or 3 amino acid variations (e.g., substitution, deletion, addition, etc.); and the amino acid sequence of CDR-L3 comprises or consists of the amino acid sequence of a CDR-L3 set forth in Table 7, or the amino acid sequence of a CDR-L3 set forth in Table 7 comprising or consisting of 1, 2, or 3 amino acid variations (e.g., substitution, deletion, addition, etc.).

In some embodiments, the amino acid sequence of CDR-H1 comprises the amino acid sequence of a CDR-H1 of a VH set forth in Table 7, or the amino acid sequence of a CDR-H1 of a VH set forth in Table 7 comprising or consisting of 1, 2, or 3 amino acid variations (e.g., substitution, deletion, addition, etc.); the amino acid sequence of CDR-H2 comprises the amino acid sequence of a CDR-H2 of a VH set forth in Table 7, or the amino acid sequence of a CDR-H2 of a VH set forth in Table 7 comprising or consisting of 1, 2, or 3 amino acid variations (e.g., substitution, deletion, addition, etc.); the amino acid sequence of CDR-H3 comprises the amino acid sequence of a CDR-H3 of a VH set forth in Table 7, or the amino acid sequence of a CDR-H3 of a VH set forth in Table 7 comprising or consisting of 1, 2, or 3 amino acid variations (e.g., substitution, deletion, addition, etc.); the amino acid sequence of CDR-L1 comprises the amino acid sequence of a CDR-L1 of a VL set forth in Table 7, or the amino acid sequence of a CDR-L1 of a VL set forth in Table 7 comprising or consisting of 1, 2, or 3 amino acid variations (e.g., substitution, deletion, addition, etc.); the amino acid sequence of CDR-L2 comprises the amino acid sequence of a CDR-L2 of a VL set forth in Table 7, or the amino acid sequence of a CDR-L2 of a VL set forth in Table 7 comprising or consisting of 1, 2, or 3 amino acid variations (e.g., substitution, deletion, addition, etc.); and the amino acid sequence of CDR-L3 comprises the amino acid sequence of a CDR-L3 of a VL set forth in Table 7, or the amino acid sequence of a CDR-L3 of a VL set forth in Table 7 comprising or consisting of 1, 2, or 3 amino acid variations (e.g., substitution, deletion, addition, etc.).

In some embodiments, the amino acid sequence of CDR-H1 comprises the amino acid sequence of a CDR-H1 set forth in Table 7, or the amino acid sequence of a CDR-H1 set forth in Table 7 comprising or consisting of 1, 2, or 3 amino acid variations (e.g., substitution, deletion, addition, etc.); the amino acid sequence of CDR-H2 comprises the amino acid sequence of a CDR-H2 set forth in Table 7, or the amino acid sequence of a CDR-H2 set forth in Table 7 comprising or consisting of 1, 2, or 3 amino acid variations (e.g., substitution, deletion, addition, etc.); the amino acid sequence of CDR-H3 comprises the amino acid sequence of a CDR-H3 set forth in Table 7, or the amino acid sequence of a CDR-H3 set forth in Table 7 comprising or consisting of 1, 2, or 3 amino acid variations (e.g., substitution, deletion, addition, etc.); the amino acid sequence of CDR-L1 comprises the amino acid sequence of a CDR-L1 set forth in Table 7, or the amino acid sequence CDR1 of a VL set forth in Table 7 comprising or consisting of 1, 2, or 3 amino acid variations (e.g., substitution, deletion, addition, etc.); the amino acid sequence of CDR-L2 comprises the amino acid sequence of a CDR-L2 set forth in Table 7, or the amino acid sequence of a CDR-L2 set forth in Table 7 comprising or consisting of 1, 2, or 3 amino acid variations (e.g., substitution, deletion, addition, etc.); and the amino acid sequence of CDR-L3 comprises the amino acid sequence of a CDR-L3 set forth in Table 7, or the amino acid sequence of a CDR-L3 set forth in Table 7 comprising or consisting of 1, 2, or 3 amino acid variations (e.g., substitution, deletion, addition, etc.).

In some embodiments, the amino acid sequence of CDR-H1 comprises the amino acid sequence of a CDR-H1 of a VH set forth in Table 7, or the amino acid sequence of a CDR-H1 of a VH set forth in Table 7 consisting of 1, 2, or 3 amino acid variations (e.g., substitution, deletion, addition, etc.); the amino acid sequence of CDR-H2 comprises the amino acid sequence of a CDR-H2 of a VH set forth in Table 7, or the amino acid sequence of a CDR-H2 of a VH set forth in Table 7 consisting of 1, 2, or 3 amino acid variations (e.g., substitution, deletion, addition, etc.); the amino acid sequence of CDR-H3 comprises the amino acid sequence of a CDR-H3 of a VH set forth in Table 7, or the amino acid sequence of a CDR-H3 of a VH set forth in Table 7 consisting of 1, 2, or 3 amino acid variations (e.g., substitution, deletion, addition, etc.); the amino acid sequence of CDR-L1 comprises the amino acid sequence of a CDR-L1 of a VL set forth in Table 7, or the amino acid sequence of a CDR-L1 of a VL set forth in Table 7 consisting of 1, 2, or 3 amino acid variations (e.g., substitution, deletion, addition, etc.); the amino acid sequence of CDR-L2 comprises the amino acid sequence of a CDR-L2 of a VL set forth in Table 7, or the amino acid sequence of a CDR-L2 of a VL set forth in Table 7 consisting of 1, 2, or 3 amino acid variations (e.g., substitution, deletion, addition, etc.); and the amino acid sequence of CDR-L3 comprises the amino acid sequence of a CDR-L3 of a VL set forth in Table 7, or the amino acid sequence of a CDR-L3 of a VL set forth in Table 7 consisting of 1, 2, or 3 amino acid variations (e.g., substitution, deletion, addition, etc.).

In some embodiments, the amino acid sequence of CDR-H1 comprises the amino acid sequence of a CDR-H1 set forth in Table 7, or the amino acid sequence of a CDR-H1 set forth in Table 7 consisting of 1, 2, or 3 amino acid variations (e.g., substitution, deletion, addition, etc.); the amino acid sequence of CDR-H2 comprises the amino acid sequence of a CDR-H2 set forth in Table 7, or the amino acid sequence of a CDR-H2 set forth in Table 7 consisting of 1, 2, or 3 amino acid variations (e.g., substitution, deletion, addition, etc.); the amino acid sequence of CDR-H3 comprises the amino acid sequence of a CDR-H3 set forth in Table 7, or the amino acid sequence of a CDR-H3 set forth in Table 7 consisting of 1, 2, or 3 amino acid variations (e.g., substitution, deletion, addition, etc.); the amino acid sequence of CDR-L1 comprises the amino acid sequence of a CDR-L1 set forth in Table 7, or the amino acid sequence CDR1 of a VL set forth in Table 7 consisting of 1, 2, or 3 amino acid variations (e.g., substitution, deletion, addition, etc.); the amino acid sequence of CDR-L2 comprises the amino acid sequence of a CDR-L2 set forth in Table 7, or the amino acid sequence of a CDR-L2 set forth in Table 7 consisting of 1, 2, or 3 amino acid variations (e.g., substitution, deletion, addition, etc.); and the amino acid sequence of CDR-L3 comprises the amino acid sequence of a CDR-L3 set forth in Table 7, or the amino acid sequence of a CDR-L3 set forth in Table 7 consisting of 1, 2, or 3 amino acid variations (e.g., substitution, deletion, addition, etc.).

In some embodiments, the amino acid sequence of CDR-H1 comprises the amino acid sequence of a CDR-H1 of a VH set forth in Table 7; the amino acid sequence of CDR-H2 comprises the amino acid sequence of a CDR-H2 of a VH set forth in Table 7; the amino acid sequence of CDR-H3 comprises the amino acid sequence of a CDR-H3 of a VH set forth in Table 7; the amino acid sequence of CDR-L1 comprises the amino acid sequence of a CDR-L1 of a VL set forth in Table 7; the amino acid sequence of CDR-L2 comprises the amino acid sequence of a CDR-L2 of a VL set forth in Table 7; and the amino acid sequence of CDR-L3 comprises the amino acid sequence of a CDR-L3 of a VL set forth in Table 7.

In some embodiments, the amino acid sequence of CDR-H1 comprises the amino acid sequence of a CDR-H1 set forth in Table 7; the amino acid sequence of CDR-H2 comprises the amino acid sequence of a CDR-H2 set forth in Table 7; the amino acid sequence of CDR-H3 comprises the amino acid sequence of a CDR-H3 set forth in Table 7; the amino acid sequence of CDR-L1 comprises the amino acid sequence of a CDR-L1 set forth in Table 7; the amino acid sequence of CDR-L2 comprises the amino acid sequence of a CDR-L2 set forth in Table 7; and the amino acid sequence of CDR-L3 comprises the amino acid sequence of a CDR-L3 set forth in Table 7.

In some embodiments, the amino acid sequence of CDR-H1 consists of the amino acid sequence of a CDR-H1 of a VH set forth in Table 7, or the amino acid sequence of a CDR-H1 of a VH set forth in Table 7 comprising or consisting of 1, 2, or 3 amino acid variations (e.g., substitution, deletion, addition, etc.); the amino acid sequence of CDR-H2 consists of the amino acid sequence of a CDR-H2 of a VH set forth in Table 7, or the amino acid sequence of a CDR-H2 of a VH set forth in Table 7 comprising or consisting of 1, 2, or 3 amino acid variations (e.g., substitution, deletion, addition, etc.); the amino acid sequence of CDR-H3 consists of the amino acid sequence of a CDR-H3 of a VH set forth in Table 7, or the amino acid sequence of a CDR-H3 of a VH set forth in Table 7 comprising or consisting of 1, 2, or 3 amino acid variations (e.g., substitution, deletion, addition, etc.); the amino acid sequence of CDR-L1 consists of the amino acid sequence of a CDR-L1 of a VL set forth in Table 7, or the amino acid sequence of a CDR-L1 of a VL set forth in Table 7 comprising or consisting of 1, 2, or 3 amino acid variations (e.g., substitution, deletion, addition, etc.); the amino acid sequence of CDR-L2 consists of the amino acid sequence of a CDR-L2 of a VL set forth in Table 7, or the amino acid sequence of a CDR-L2 of a VL set forth in Table 7 comprising or consisting of 1, 2, or 3 amino acid variations (e.g., substitution, deletion, addition, etc.); and the amino acid sequence of CDR-L3 consists of the amino acid sequence of a CDR-L3 of a VL set forth in Table 7, or the amino acid sequence of a CDR-L3 of a VL set forth in Table 7 comprising or consisting of 1, 2, or 3 amino acid variations (e.g., substitution, deletion, addition, etc.).

In some embodiments, the amino acid sequence of CDR-H1 consists of the amino acid sequence of a CDR-H1 set forth in Table 7, or the amino acid sequence of a CDR-H1 set forth in Table 7 comprising or consisting of 1, 2, or 3 amino acid variations (e.g., substitution, deletion, addition, etc.); the amino acid sequence of CDR-H2 consists of the amino acid sequence of a CDR-H2 set forth in Table 7, or the amino acid sequence of a CDR-H2 set forth in Table 7 comprising or consisting of 1, 2, or 3 amino acid variations (e.g., substitution, deletion, addition, etc.); the amino acid sequence of CDR-H3 consists of the amino acid sequence of a CDR-H3 set forth in Table 7, or the amino acid sequence of a CDR-H3 set forth in Table 7 comprising or consisting of 1, 2, or 3 amino acid variations (e.g., substitution, deletion, addition, etc.); the amino acid sequence of CDR-L1 consists of the amino acid sequence of a CDR-L1 set forth in Table 7, or the amino acid sequence CDR1 of a VL set forth in Table 7 comprising or consisting of 1, 2, or 3 amino acid variations (e.g., substitution, deletion, addition, etc.); the amino acid sequence of CDR-L2 consists of the amino acid sequence of a CDR-L2 set forth in Table 7, or the amino acid sequence of a CDR-L2 set forth in Table 7 comprising or consisting of 1, 2, or 3 amino acid variations (e.g., substitution, deletion, addition, etc.); and the amino acid sequence of CDR-L3 consists of the amino acid sequence of a CDR-L3 set forth in Table 7, or the amino acid sequence of a CDR-L3 set forth in Table 7 comprising or consisting of 1, 2, or 3 amino acid variations (e.g., substitution, deletion, addition, etc.).

In some embodiments, the amino acid sequence of CDR-H1 consists of the amino acid sequence of a CDR-H1 of a VH set forth in Table 7, or the amino acid sequence of a CDR-H1 of a VH set forth in Table 7 consisting of 1, 2, or 3 amino acid variations (e.g., substitution, deletion, addition, etc.); the amino acid sequence of CDR-H2 consists of the amino acid sequence of a CDR-H2 of a VH set forth in Table 7, or the amino acid sequence of a CDR-H2 of a VH set forth in Table 7 consisting of 1, 2, or 3 amino acid variations (e.g., substitution, deletion, addition, etc.); the amino acid sequence of CDR-H3 consists of the amino acid sequence of a CDR-H3 of a VH set forth in Table 7, or the amino acid sequence of a CDR-H3 of a VH set forth in Table 7 consisting of 1, 2, or 3 amino acid variations (e.g., substitution, deletion, addition, etc.); the amino acid sequence of CDR-L1 consists of the amino acid sequence of a CDR-L1 of a VL set forth in Table 7, or the amino acid sequence of a CDR-L1 of a VL set forth in Table 7 consisting of 1, 2, or 3 amino acid variations (e.g., substitution, deletion, addition, etc.); the amino acid sequence of CDR-L2 consists of the amino acid sequence of a CDR-L2 of a VL set forth in Table 7, or the amino acid sequence of a CDR-L2 of a VL set forth in Table 7 consisting of 1, 2, or 3 amino acid variations (e.g., substitution, deletion, addition, etc.); and the amino acid sequence of CDR-L3 consists of the amino acid sequence of a CDR-L3 of a VL set forth in Table 7, or the amino acid sequence of a CDR-L3 of a VL set forth in Table 7 consisting of 1, 2, or 3 amino acid variations (e.g., substitution, deletion, addition, etc.).

In some embodiments, the amino acid sequence of CDR-H1 consists of the amino acid sequence of a CDR-H1 set forth in Table 7, or the amino acid sequence of a CDR-H1 set forth in Table 7 consisting of 1, 2, or 3 amino acid variations (e.g., substitution, deletion, addition, etc.); the amino acid sequence of CDR-H2 consists of the amino acid sequence of a CDR-H2 set forth in Table 7, or the amino acid sequence of a CDR-H2 set forth in Table 7 consisting of 1, 2, or 3 amino acid variations (e.g., substitution, deletion, addition, etc.); the amino acid sequence of CDR-H3 consists of the amino acid sequence of a CDR-H3 set forth in Table 7, or the amino acid sequence of a CDR-H3 set forth in Table 7 consisting of 1, 2, or 3 amino acid variations (e.g., substitution, deletion, addition, etc.); the amino acid sequence of CDR-L1 consists of the amino acid sequence of a CDR-L1 set forth in Table 7, or the amino acid sequence CDR1 of a VL set forth in Table 7 consisting of 1, 2, or 3 amino acid variations (e.g., substitution, deletion, addition, etc.); the amino acid sequence of CDR-L2 consists of the amino acid sequence of a CDR-L2 set forth in Table 7, or the amino acid sequence of a CDR-L2 set forth in Table 7 consisting of 1, 2, or 3 amino acid variations (e.g., substitution, deletion, addition, etc.); and the amino acid sequence of CDR-L3 consists of the amino acid sequence of a CDR-L3 set forth in Table 7, or the amino acid sequence of a CDR-L3 set forth in Table 7 consisting of 1, 2, or 3 amino acid variations (e.g., substitution, deletion, addition, etc.).

In some embodiments, the amino acid sequence of CDR-H1 consists of the amino acid sequence of a CDR-H1 of a VH set forth in Table 7; the amino acid sequence of CDR-H2 consists of the amino acid sequence of a CDR-H2 of a VH set forth in Table 7; the amino acid sequence of CDR-H3 consists of the amino acid sequence of a CDR-H3 of a VH set forth in Table 7; the amino acid sequence of CDR-L1 consists of the amino acid sequence of a CDR-L1 of a VL set forth in Table 7; the amino acid sequence of CDR-L2 consists of the amino acid sequence of a CDR-L2 of a VL set forth in Table 7; and the amino acid sequence of CDR-L3 consists of the amino acid sequence of a CDR-L3 of a VL set forth in Table 7.

In some embodiments, the amino acid sequence of CDR-H1 consists of the amino acid sequence of a CDR-H1 set forth in Table 7; the amino acid sequence of CDR-H2 consists of the amino acid sequence of a CDR-H2 set forth in Table 7; the amino acid sequence of CDR-H3 consists of the amino acid sequence of a CDR-H3 set forth in Table 7; the amino acid sequence of CDR-L1 consists of the amino acid sequence of a CDR-L1 set forth in Table 7; the amino acid sequence of CDR-L2 consists of the amino acid sequence of a CDR-L2 set forth in Table 7; and the amino acid sequence of CDR-L3 consists of the amino acid sequence of a CDR-L3 set forth in Table 7.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 7. In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 7. In some embodiments, the amino acid sequence of the VH comprises an amino acid sequence at least 90%, 91%, 92%, 93%, 94%, 95%, 96%, 97%, 98%, 99%, or 100% identical to the amino acid sequence of a VH set forth in Table 7. In some embodiments, the amino acid sequence of the VH comprises an amino acid sequence 100% identical to the amino acid sequence of a VH set forth in Table 7. In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 7. In some embodiments, the amino acid sequence of the VH consists of an amino acid sequence at least 90%, 91%, 92%, 93%, 94%, 95%, 96%, 97%, 98%, 99%, or 100% identical to the amino acid sequence of a VH set forth in Table 7. In some embodiments, the amino acid sequence of the VH consists of an amino acid sequence 100% identical to the amino acid sequence of a VH set forth in Table 7.

In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 7. In some embodiments, the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 7. In some embodiments, the amino acid sequence of the VL comprises an amino acid sequence at 90%, 91%, 92%, 93%, 94%, 95%, 96%, 97%, 98%, 99%, or 100% identical to the amino acid sequence of a VL set forth in Table 7. In some embodiments, the amino acid sequence of the VL comprises an amino acid sequence 100% identical to the amino acid sequence of a VL set forth in Table 7. In some embodiments, the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 7. In some embodiments, the amino acid sequence of the VL consists of an amino acid sequence at least 90%, 91%, 92%, 93%, 94%, 95%, 96%, 97%, 98%, 99%, or 100% identical to the amino acid sequence of a VL set forth in Table 7. In some embodiments, the amino acid sequence of the VL consists of an amino acid sequence 100% identical to the amino acid sequence of a VL set forth in Table 7.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 7; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 7.

In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 7; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 7. In some embodiments, the amino acid sequence of the VH comprises an amino acid sequence at least 90%, 91%, 92%, 93%, 94%, 95%, 96%, 97%, 98%, 99%, or 100% identical to the amino acid sequence of a VH set forth in Table 7; and the amino acid sequence of the VL comprises an amino acid sequence at least 90%, 91%, 92%, 93%, 94%, 95%, 96%, 97%, 98%, 99%, or 100% identical to the amino acid sequence of a VL set forth in Table 7. In some embodiments, the amino acid sequence of the VH comprises an amino acid sequence 100% identical to the amino acid sequence of a VH set forth in Table 7; and the amino acid sequence of the VL comprises an amino acid sequence 100% identical to the amino acid sequence of a VL set forth in Table 7.

In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VH set forth in Table 7; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a VL set forth in Table 7. In some embodiments, the amino acid sequence of the VH consists of an amino acid sequence at least 90%, 91%, 92%, 93%, 94%, 95%, 96%, 97%, 98%, 99%, or 100% identical to the amino acid sequence of a VH set forth in Table 7; and the amino acid sequence of the VL consists of an amino acid sequence at least 90%, 91%, 92%, 93%, 94%, 95%, 96%, 97%, 98%, 99%, or 100% identical to the amino acid sequence of a VL set forth in Table 7. In some embodiments, the amino acid sequence of the VH consists of an amino acid sequence 100% identical to the amino acid sequence of a VH set forth in Table 7; and the amino acid sequence of the VL consists of an amino acid sequence 100% identical to the amino acid sequence of a VL set forth in Table 7.

In some embodiments, the amino acid sequence of the HC comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 7. In some embodiments, the amino acid sequence of the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 7. In some embodiments, the amino acid sequence of the HC comprises an amino acid sequence at least 90%, 91%, 92%, 93%, 94%, 95%, 96%, 97%, 98%, 99%, or 100% identical to the amino acid sequence of a HC set forth in Table 7. In some embodiments, the amino acid sequence of the HC comprises an amino acid sequence 100% identical to the amino acid sequence of a HC set forth in Table 7. In some embodiments, the amino acid sequence of the HC consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 7. In some embodiments, the amino acid sequence of the HC consists of an amino acid sequence at least 90%, 91%, 92%, 93%, 94%, 95%, 96%, 97%, 98%, 99%, or 100% identical to the amino acid sequence of a HC set forth in Table 7. In some embodiments, the amino acid sequence of the HC consists of an amino acid sequence 100% identical to the amino acid sequence of a HC set forth in Table 7.

In some embodiments, the amino acid sequence of the LC comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 7. In some embodiments, the amino acid sequence of the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 7. In some embodiments, the amino acid sequence of the LC comprises an amino acid sequence at 90%, 91%, 92%, 93%, 94%, 95%, 96%, 97%, 98%, 99%, or 100% identical to the amino acid sequence of a LC set forth in Table 7. In some embodiments, the amino acid sequence of the LC comprises an amino acid sequence 100% identical to the amino acid sequence of a LC set forth in Table 7. In some embodiments, the amino acid sequence of the LC consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 7. In some embodiments, the amino acid sequence of the LC consists of an amino acid sequence at least 90%, 91%, 92%, 93%, 94%, 95%, 96%, 97%, 98%, 99%, or 100% identical to the amino acid sequence of a LC set forth in Table 7. In some embodiments, the amino acid sequence of the LC consists of an amino acid sequence 100% identical to the amino acid sequence of a LC set forth in Table 7.

In some embodiments, the amino acid sequence of the HC comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 7; and the amino acid sequence of the LC comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 7.

In some embodiments, the amino acid sequence of the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 7; and the amino acid sequence of the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 7. In some embodiments, the amino acid sequence of the HC comprises an amino acid sequence at least 90%, 91%, 92%, 93%, 94%, 95%, 96%, 97%, 98%, 99%, or 100% identical to the amino acid sequence of a HC set forth in Table 7; and the amino acid sequence of the LC comprises an amino acid sequence at least 90%, 91%, 92%, 93%, 94%, 95%, 96%, 97%, 98%, 99%, or 100% identical to the amino acid sequence of a LC set forth in Table 7. In some embodiments, the amino acid sequence of the HC comprises an amino acid sequence 100% identical to the amino acid sequence of a HC set forth in Table 7; and the amino acid sequence of the LC comprises an amino acid sequence 100% identical to the amino acid sequence of a LC set forth in Table 7.

In some embodiments, the amino acid sequence of the HC consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a HC set forth in Table 7; and the amino acid sequence of the LC consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a LC set forth in Table 7. In some embodiments, the amino acid sequence of the HC consists of an amino acid sequence at least 90%, 91%, 92%, 93%, 94%, 95%, 96%, 97%, 98%, 99%, or 100% identical to the amino acid sequence of a HC set forth in Table 7; and the amino acid sequence of the LC consists of an amino acid sequence at least 90%, 91%, 92%, 93%, 94%, 95%, 96%, 97%, 98%, 99%, or 100% identical to the amino acid sequence of a LC set forth in Table 7. In some embodiments, the amino acid sequence of the HC consists of an amino acid sequence 100% identical to the amino acid sequence of a HC set forth in Table 7; and the amino acid sequence of the LC consists of an amino acid sequence 100% identical to the amino acid sequence of a LC set forth in Table 7.

In some embodiments, the integrin (e.g., α4β7 integrin) binding domain comprises a VH that comprises: a CDR-H1, a CDR-H2, and a CDR-H3; and VL that comprises CDR-L1, a CDR-L2, and a CDR-L3.

In some embodiments, the amino acid sequence of CDR-H1 comprises or consists of the amino acid sequence set forth in SEQ ID NO: 583, or the amino acid sequence set forth in SEQ ID NO: 583 comprising or consisting of 1, 2, or 3 amino acid modifications (e.g., substitution, deletion, addition, etc.); the amino acid sequence of CDR-H2 comprises or consists of the amino acid sequence set forth in SEQ ID NO: 584, or the amino acid sequence set forth in SEQ ID NO: 584 comprising or consisting of 1, 2, or 3 amino acid modifications (e.g., substitution, deletion, addition, etc.); the amino acid sequence of CDR-H3 comprises or consists of the amino acid sequence set forth in SEQ ID NO: 585, or the amino acid sequence set forth in SEQ ID NO: 585 comprising or consisting of 1, 2, or 3 amino acid modifications (e.g., substitution, deletion, addition, etc.); the amino acid sequence of CDR-L1 comprises or consists of the amino acid sequence set forth in SEQ ID NO: 586, or the amino acid sequence set forth in SEQ ID NO: 586 comprising or consisting of 1, 2, or 3 amino acid modifications (e.g., substitution, deletion, addition, etc.); the amino acid sequence of CDR-L2 comprises or consists of the amino acid sequence set forth in SEQ ID NO: 587, or the amino acid sequence set forth in SEQ ID NO: 587 comprising or consisting of 1, 2, or 3 amino acid modifications (e.g., substitution, deletion, addition, etc.); and the amino acid sequence of CDR-L3 comprises or consists of the amino acid sequence set forth in SEQ ID NO: 588, or the amino acid sequence set forth in SEQ ID NO: 588 comprising or consisting of 1, 2, or 3 amino acid modifications (e.g., substitution, deletion, addition, etc.).

In some embodiments, the amino acid sequence of CDR-H1 comprises the amino acid sequence set forth in SEQ ID NO: 583, or the amino acid sequence set forth in SEQ ID NO: 583 comprising or consisting of 1, 2, or 3 amino acid modifications (e.g., substitution, deletion, addition, etc.); the amino acid sequence of CDR-H2 comprises the amino acid sequence set forth in SEQ ID NO: 584, or the amino acid sequence set forth in SEQ ID NO: 584 comprising or consisting of 1, 2, or 3 amino acid modifications (e.g., substitution, deletion, addition, etc.); the amino acid sequence of CDR-H3 comprises the amino acid sequence set forth in SEQ ID NO: 585, or the amino acid sequence set forth in SEQ ID NO: 585 comprising or consisting of 1, 2, or 3 amino acid modifications (e.g., substitution, deletion, addition, etc.); the amino acid sequence of CDR-L1 comprises the amino acid sequence set forth in SEQ ID NO: 586, or the amino acid sequence set forth in SEQ ID NO: 586 comprising or consisting of 1, 2, or 3 amino acid modifications (e.g., substitution, deletion, addition, etc.); the amino acid sequence of CDR-L2 comprises the amino acid sequence set forth in SEQ ID NO: 587, or the amino acid sequence set forth in SEQ ID NO: 587 comprising or consisting of 1, 2, or 3 amino acid modifications (e.g., substitution, deletion, addition, etc.); and the amino acid sequence of CDR-L3 comprises the amino acid sequence set forth in SEQ ID NO: 588, or the amino acid sequence set forth in SEQ ID NO: 588 comprising or consisting of 1, 2, or 3 amino acid modifications (e.g., substitution, deletion, addition, etc.).

In some embodiments, the amino acid sequence of CDR-H1 comprises the amino acid sequence set forth in SEQ ID NO: 583, or the amino acid sequence set forth in SEQ ID NO: 583 comprising 1, 2, or 3 amino acid modifications (e.g., substitution, deletion, addition, etc.); the amino acid sequence of CDR-H2 comprises the amino acid sequence set forth in SEQ ID NO: 584, or the amino acid sequence set forth in SEQ ID NO: 584 comprising 1, 2, or 3 amino acid modifications (e.g., substitution, deletion, addition, etc.); the amino acid sequence of CDR-H3 comprises the amino acid sequence set forth in SEQ ID NO: 585, or the amino acid sequence set forth in SEQ ID NO: 585 comprising 1, 2, or 3 amino acid modifications (e.g., substitution, deletion, addition, etc.); the amino acid sequence of CDR-L1 comprises the amino acid sequence set forth in SEQ ID NO: 586, or the amino acid sequence set forth in SEQ ID NO: 586 comprising 1, 2, or 3 amino acid modifications (e.g., substitution, deletion, addition, etc.); the amino acid sequence of CDR-L2 comprises the amino acid sequence set forth in SEQ ID NO: 587, or the amino acid sequence set forth in SEQ ID NO: 587 comprising 1, 2, or 3 amino acid modifications (e.g., substitution, deletion, addition, etc.); and the amino acid sequence of CDR-L3 comprises the amino acid sequence set forth in SEQ ID NO: 588, or the amino acid sequence set forth in SEQ ID NO: 588 comprising 1, 2, or 3 amino acid modifications (e.g., substitution, deletion, addition, etc.).

In some embodiments, the amino acid sequence of CDR-H1 comprises the amino acid sequence set forth in SEQ ID NO: 583, or the amino acid sequence set forth in SEQ ID NO: 583 consisting of 1, 2, or 3 amino acid modifications (e.g., substitution, deletion, addition, etc.); the amino acid sequence of CDR-H2 comprises the amino acid sequence set forth in SEQ ID NO: 584, or the amino acid sequence set forth in SEQ ID NO: 584 consisting of 1, 2, or 3 amino acid modifications (e.g., substitution, deletion, addition, etc.); the amino acid sequence of CDR-H3 comprises the amino acid sequence set forth in SEQ ID NO: 585, or the amino acid sequence set forth in SEQ ID NO: 585 consisting of 1, 2, or 3 amino acid modifications (e.g., substitution, deletion, addition, etc.); the amino acid sequence of CDR-L1 comprises the amino acid sequence set forth in SEQ ID NO: 586, or the amino acid sequence set forth in SEQ ID NO: 586 consisting of 1, 2, or 3 amino acid modifications (e.g., substitution, deletion, addition, etc.); the amino acid sequence of CDR-L2 comprises the amino acid sequence set forth in SEQ ID NO: 587, or the amino acid sequence set forth in SEQ ID NO: 587 consisting of 1, 2, or 3 amino acid modifications (e.g., substitution, deletion, addition, etc.); and the amino acid sequence of CDR-L3 comprises the amino acid sequence set forth in SEQ ID NO: 588, or the amino acid sequence set forth in SEQ ID NO: 588 consisting of 1, 2, or 3 amino acid modifications (e.g., substitution, deletion, addition, etc.).

In some embodiments, the amino acid sequence of CDR-H1 comprises the amino acid sequence set forth in SEQ ID NO: 583; the amino acid sequence of CDR-H2 comprises the amino acid sequence set forth in SEQ ID NO: 584; the amino acid sequence of CDR-H3 comprises the amino acid sequence set forth in SEQ ID NO: 585; the amino acid sequence of CDR-L1 comprises the amino acid sequence set forth in SEQ ID NO: 586; the amino acid sequence of CDR-L2 comprises the amino acid sequence set forth in SEQ ID NO: 587; and the amino acid sequence of CDR-L3 comprises the amino acid sequence set forth in SEQ ID NO: 588.

In some embodiments, the amino acid sequence of CDR-H1 consists of the amino acid sequence set forth in SEQ ID NO: 583, or the amino acid sequence set forth in SEQ ID NO: 583 comprising or consisting of 1, 2, or 3 amino acid modifications (e.g., substitution, deletion, addition, etc.); the amino acid sequence of CDR-H2 consists of the amino acid sequence set forth in SEQ ID NO: 584, or the amino acid sequence set forth in SEQ ID NO: 584 comprising or consisting of 1, 2, or 3 amino acid modifications (e.g., substitution, deletion, addition, etc.); the amino acid sequence of CDR-H3 consists of the amino acid sequence set forth in SEQ ID NO: 585, or the amino acid sequence set forth in SEQ ID NO: 585 comprising or consisting of 1, 2, or 3 amino acid modifications (e.g., substitution, deletion, addition, etc.); the amino acid sequence of CDR-L1 consists of the amino acid sequence set forth in SEQ ID NO: 586, or the amino acid sequence set forth in SEQ ID NO: 586 comprising or consisting of 1, 2, or 3 amino acid modifications (e.g., substitution, deletion, addition, etc.); the amino acid sequence of CDR-L2 consists of the amino acid sequence set forth in SEQ ID NO: 587, or the amino acid sequence set forth in SEQ ID NO: 587 comprising or consisting of 1, 2, or 3 amino acid modifications (e.g., substitution, deletion, addition, etc.); and the amino acid sequence of CDR-L3 consists of the amino acid sequence set forth in SEQ ID NO: 588, or the amino acid sequence set forth in SEQ ID NO: 588 comprising or consisting of 1, 2, or 3 amino acid modifications (e.g., substitution, deletion, addition, etc.).

In some embodiments, the amino acid sequence of CDR-H1 consists of the amino acid sequence set forth in SEQ ID NO: 583, or the amino acid sequence set forth in SEQ ID NO: 583 comprising 1, 2, or 3 amino acid modifications (e.g., substitution, deletion, addition, etc.); the amino acid sequence of CDR-H2 consists of the amino acid sequence set forth in SEQ ID NO: 584, or the amino acid sequence set forth in SEQ ID NO: 584 comprising 1, 2, or 3 amino acid modifications (e.g., substitution, deletion, addition, etc.); the amino acid sequence of CDR-H3 consists of the amino acid sequence set forth in SEQ ID NO: 585, or the amino acid sequence set forth in SEQ ID NO: 585 comprising 1, 2, or 3 amino acid modifications (e.g., substitution, deletion, addition, etc.); the amino acid sequence of CDR-L1 consists of the amino acid sequence set forth in SEQ ID NO: 586, or the amino acid sequence set forth in SEQ ID NO: 586 comprising 1, 2, or 3 amino acid modifications (e.g., substitution, deletion, addition, etc.); the amino acid sequence of CDR-L2 consists of the amino acid sequence set forth in SEQ ID NO: 587, or the amino acid sequence set forth in SEQ ID NO: 587 comprising 1, 2, or 3 amino acid modifications (e.g., substitution, deletion, addition, etc.); and the amino acid sequence of CDR-L3 consists of the amino acid sequence set forth in SEQ ID NO: 588, or the amino acid sequence set forth in SEQ ID NO: 588 comprising 1, 2, or 3 amino acid modifications (e.g., substitution, deletion, addition, etc.).

In some embodiments, the amino acid sequence of CDR-H1 consists of the amino acid sequence set forth in SEQ ID NO: 583, or the amino acid sequence set forth in SEQ ID NO: 583 consisting of 1, 2, or 3 amino acid modifications (e.g., substitution, deletion, addition, etc.); the amino acid sequence of CDR-H2 consists of the amino acid sequence set forth in SEQ ID NO: 584, or the amino acid sequence set forth in SEQ ID NO: 584 consisting of 1, 2, or 3 amino acid modifications (e.g., substitution, deletion, addition, etc.); the amino acid sequence of CDR-H3 consists of the amino acid sequence set forth in SEQ ID NO: 585, or the amino acid sequence set forth in SEQ ID NO: 585 consisting of 1, 2, or 3 amino acid modifications (e.g., substitution, deletion, addition, etc.); the amino acid sequence of CDR-L1 consists of the amino acid sequence set forth in SEQ ID NO: 586, or the amino acid sequence set forth in SEQ ID NO: 586 consisting of 1, 2, or 3 amino acid modifications (e.g., substitution, deletion, addition, etc.); the amino acid sequence of CDR-L2 consists of the amino acid sequence set forth in SEQ ID NO: 587, or the amino acid sequence set forth in SEQ ID NO: 587 consisting of 1, 2, or 3 amino acid modifications (e.g., substitution, deletion, addition, etc.); and the amino acid sequence of CDR-L3 consists of the amino acid sequence set forth in SEQ ID NO: 588, or the amino acid sequence set forth in SEQ ID NO: 588 consisting of 1, 2, or 3 amino acid modifications (e.g., substitution, deletion, addition, etc.).

In some embodiments, the amino acid sequence of CDR-H1 consists of the amino acid sequence set forth in SEQ ID NO: 583; the amino acid sequence of CDR-H2 consists of the amino acid sequence set forth in SEQ ID NO: 584; the amino acid sequence of CDR-H3 consists of the amino acid sequence set forth in SEQ ID NO: 585; the amino acid sequence of CDR-L1 consists of the amino acid sequence set forth in SEQ ID NO: 586; the amino acid sequence of CDR-L2 consists of the amino acid sequence set forth in SEQ ID NO: 587; and the amino acid sequence of CDR-L3 comprises the amino acid sequence set forth in SEQ ID NO: 588.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 589. In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 589. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 589. In some embodiments, the amino acid sequence of the VH comprises an amino acid sequence 100% identical to the amino acid sequence set forth in SEQ ID NO: 589. In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 589. In some embodiments, the amino acid sequence of the VH consists of an amino acid sequence at least 90%, 91%, 92%, 93%, 94%, 95%, 96%, 97%, 98%, 99%, or 100% identical to the amino acid sequence set forth in SEQ ID NO: 589. In some embodiments, the amino acid sequence of the VH consists of an amino acid sequence 100% identical to the amino acid sequence set forth in SEQ ID NO: 589.

In some embodiments, the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 590. In some embodiments, the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 590. In some embodiments, the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 590. In some embodiments, the amino acid sequence of the VL comprises an amino acid sequence 100% identical to the amino acid sequence set forth in SEQ ID NO: 590. In some embodiments, the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 590. In some embodiments, the amino acid sequence of the VL consists of an amino acid sequence at least 90%, 91%, 92%, 93%, 94%, 95%, 96%, 97%, 98%, 99%, or 100% identical to the amino acid sequence set forth in SEQ ID NO: 590. In some embodiments, the amino acid sequence of the VL consists of an amino acid sequence 100% identical to the amino acid sequence set forth in SEQ ID NO: 590.

In some embodiments, the amino acid sequence of the VH comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 589; and the amino acid sequence of the VL comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 590. In some embodiments, the amino acid sequence of the VH comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 589; and the amino acid sequence of the VL comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 590. In some embodiments, the amino acid sequence of the VH comprises an amino acid sequence at least 90%, 91%, 92%, 93%, 94%, 95%, 96%, 97%, 98%, 99%, or 100% identical to the amino acid sequence set forth in SEQ ID NO: 589; and the amino acid sequence of the VL comprises an amino acid sequence at least 90%, 91%, 92%, 93%, 94%, 95%, 96%, 97%, 98%, 99%, or 100% identical to the amino acid sequence set forth in SEQ ID NO: 590. In some embodiments, the amino acid sequence of the VH comprises an amino acid sequence 100% identical to the amino acid sequence set forth in SEQ ID NO: 589; and the amino acid sequence of the VL comprises an amino acid sequence 100% identical to the amino acid sequence set forth in SEQ ID NO: 590. In some embodiments, the amino acid sequence of the VH consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 589; and the amino acid sequence of the VL consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 590. In some embodiments, the amino acid sequence of the VH consists of an amino acid sequence at least 90%, 91%, 92%, 93%, 94%, 95%, 96%, 97%, 98%, 99%, or 100% identical to the amino acid sequence set forth in SEQ ID NO: 589; and the amino acid sequence of the VL consists of an amino acid sequence at least 90%, 91%, 92%, 93%, 94%, 95%, 96%, 97%, 98%, 99%, or 100% identical to the amino acid sequence set forth in SEQ ID NO: 590. In some embodiments, the amino acid sequence of the VH consists of an amino acid sequence 100% identical to the amino acid sequence set forth in SEQ ID NO: 589; and the amino acid sequence of the VL consists of an amino acid sequence 100% identical to the amino acid sequence set forth in SEQ ID NO: 590.

In some embodiments, the amino acid sequence of the HC comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 591. In some embodiments, the amino acid sequence of the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 591. In some embodiments, the amino acid sequence of the HC comprises an amino acid sequence at least 90%, 91%, 92%, 93%, 94%, 95%, 96%, 97%, 98%, 99%, or 100% identical to the amino acid sequence set forth in SEQ ID NO: 591. In some embodiments, the amino acid sequence of the HC comprises an amino acid sequence 100% identical to the amino acid sequence set forth in SEQ ID NO: 591. In some embodiments, the amino acid sequence of the HC consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 591. In some embodiments, the amino acid sequence of the HC consists of an amino acid sequence at least 90%, 91%, 92%, 93%, 94%, 95%, 96%, 97%, 98%, 99%, or 100% identical to the amino acid sequence set forth in SEQ ID NO: 591. In some embodiments, the amino acid sequence of the HC consists of an amino acid sequence 100% identical to the amino acid sequence set forth in SEQ ID NO: 591.

In some embodiments, the amino acid sequence of the LC comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592. In some embodiments, the amino acid sequence of the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592. In some embodiments, the amino acid sequence of the LC comprises an amino acid sequence at least 90%, 91%, 92%, 93%, 94%, 95%, 96%, 97%, 98%, 99%, or 100% identical to the amino acid sequence set forth in SEQ ID NO: 592. In some embodiments, the amino acid sequence of the LC comprises an amino acid sequence 100% identical to the amino acid sequence set forth in SEQ ID NO: 592. In some embodiments, the amino acid sequence of the LC consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592. In some embodiments, the amino acid sequence of the LC consists of an amino acid sequence at least 90%, 91%, 92%, 93%, 94%, 95%, 96%, 97%, 98%, 99%, or 100% identical to the amino acid sequence set forth in SEQ ID NO: 592. In some embodiments, the amino acid sequence of the LC consists of an amino acid sequence 100% identical to the amino acid sequence set forth in SEQ ID NO: 592.

In some embodiments, the amino acid sequence of the HC comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 591; and the amino acid sequence of the LC comprises or consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592. In some embodiments, the amino acid sequence of the HC comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 591; and the amino acid sequence of the LC comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592. In some embodiments, the amino acid sequence of the HC comprises an amino acid sequence at least 90%, 91%, 92%, 93%, 94%, 95%, 96%, 97%, 98%, 99%, or 100% identical to the amino acid sequence set forth in SEQ ID NO: 591; and the amino acid sequence of the LC comprises an amino acid sequence at least 90%, 91%, 92%, 93%, 94%, 95%, 96%, 97%, 98%, 99%, or 100% identical to the amino acid sequence set forth in SEQ ID NO: 592. In some embodiments, the amino acid sequence of the HC comprises an amino acid sequence 100% identical to the amino acid sequence set forth in SEQ ID NO: 591; and the amino acid sequence of the LC comprises an amino acid sequence 100% identical to the amino acid sequence set forth in SEQ ID NO: 592. In some embodiments, the amino acid sequence of the HC consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 591; and the amino acid sequence of the LC consists of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592. In some embodiments, the amino acid sequence of the HC consists of an amino acid sequence at least 90%, 91%, 92%, 93%, 94%, 95%, 96%, 97%, 98%, 99%, or 100% identical to the amino acid sequence set forth in SEQ ID NO: 591; and the amino acid sequence of the LC consists of an amino acid sequence at least 90%, 91%, 92%, 93%, 94%, 95%, 96%, 97%, 98%, 99%, or 100% identical to the amino acid sequence set forth in SEQ ID NO: 592. In some embodiments, the amino acid sequence of the HC consists of an amino acid sequence 100% identical to the amino acid sequence set forth in SEQ ID NO: 591; and the amino acid sequence of the LC consists of an amino acid sequence 100% identical to the amino acid sequence set forth in SEQ ID NO: 592.

### 5.2.2.2 IL10-R Binding Proteins, TL1A Binding Proteins, TNFα Binding Proteins, and CD30L Binding Proteins

The integrin targeting immunomodulatory proteins described herein can comprise one or more of an IL-10R binding protein described herein (see, e.g., § 5.2.1.1); a TL1A binding protein described herein (see, e.g., § 5.2.1.2); a TNF binding protein described herein (see, e.g., § 5.2.1.3); and/or a CD30L binding protein described herein (see, e.g., § 5.2.1.4).

In some aspects and embodiments, an integrin targeting fusion protein described herein comprises one or more protein described herein that specifically binds the hIL-10R (e.g., hIL-10Rβ, hIL-10Rα). In some aspects and embodiments, an integrin targeting fusion protein described herein comprises one or more protein described herein that specifically binds the hIL-10R (e.g., hIL-10Rβ, hIL-10Rα) set forth in § 5.2.1.1.

In some aspects and embodiments, an integrin targeting fusion protein described herein comprises one or more protein described herein that specifically binds the hTL1A. In some aspects and embodiments, an integrin targeting fusion protein described herein comprises one or more protein described herein that specifically binds hTL1A set forth in § 5.2.1.2.

In some aspects and embodiments, an integrin targeting fusion protein described herein comprises one or more protein described herein that specifically binds the hTNF. In some aspects and embodiments, an integrin targeting fusion protein described herein comprises one or more protein described herein that specifically binds hTNF set forth in § 5.2.1.3.

In some aspects and embodiments, an integrin targeting fusion protein described herein comprises one or more protein described herein that specifically binds the hCD30L. In some aspects and embodiments, an integrin targeting fusion protein described herein comprises one or more protein described herein that specifically binds hCD30L set forth in § 5.2.1.4.

### 5.3 Half-Life Extension Moieties

In some embodiments, a fusion protein described herein comprises a half-life extension moiety (e.g., protein). Various half-life extension moieties are known in the art. See, e.g., Ko S, Jo M, Jung S T. Recent Achievements and Challenges in Prolonging the Serum Half-Lives of Therapeutic IgG Antibodies Through Fc Engineering. BioDrugs. 2021; 35(2):147-157. doi:10.1007/s40259-021-00471-0 (hereinafter “Ko 2021”); Bech, E. M., Pedersen, S. L., & Jensen, K. J. (2018). Chemical Strategies for Half-Life Extension of Biopharmaceuticals: Lipidation and Its Alternatives. ACS medicinal chemistry letters, 9(7), 577-580. https://doi.org/10.1021/acsmedchemlett.8b00226 (hereinafter “Bech 2018”); Mester S, Evers M, Meyer S, et al. Extended plasma half-life of albumin-binding domain fused human IgA upon pH-dependent albumin engagement of human FcRn in vitro and in vivo. MAbs. 2021; 13(1):1893888. doi:10.1080/19420862.2021.1893888 (hereinafter “Mester 2021”); Kontermann R E. Strategies for extended serum half-life of protein therapeutics. Curr Opin Biotechnol. 2011; 22(6):868-876. doi:10.1016/j.copbio.2011.06.012 (hereinafter “Kontermann 2011”); Strohl W. R. (2015). Fusion Proteins for Half-Life Extension of Biologics as a Strategy to Make Biobetters. BioDrugs: clinical immunotherapeutics, biopharmaceuticals and gene therapy, 29(4), 215-239. https://doi.org/10.1007/s40259-015-0133-6; Zaman R, Islam R A, Ibnat N, et al. Current strategies in extending half-lives of therapeutic proteins. J Control Release. 2019; 301:176-189. doi:10.1016/j.jconrel.2019.02.016; Chen C, Constantinou A, Chester K A, et al. Glycoengineering approach to half-life extension of recombinant biotherapeutics. Bioconjug Chem. 2012; 23(8):1524-1533. doi:10.1021/bc200624a; Gupta, Vijayalaxmi et al. “Protein PEGylation for cancer therapy: bench to bedside.” Journal of cell communication and signaling vol. 13, 3 (2019): 319-330. doi:10.1007/s12079-018-0492-0; Martin Schlapschy, et al, PASylation: a biological alternative to PEGylation for extending the plasma half-life of pharmaceutically active proteins, Protein Engineering, Design and Selection, Volume 26, Issue 8, August 2013, Pages 489-501, https://doi.org/10.1093/protein/gzt023; Strohl, William R. “Fusion Proteins for Half-Life Extension of Biologics as a Strategy to Make Biobetters.” BioDrugs: clinical immunotherapeutics, biopharmaceuticals and gene therapy vol. 29, 4 (2015): 215-39. doi:10.1007/s40259-015-0133-6; the entire contents of each of which are incorporated by reference herein for all purposes.

Exemplary half-life extension moieties include, but are not limited to, an immunoglobulin (e.g., human Ig (hIg), murine Ig (mIg)), a fragment of an Ig (e.g., hIg, mIg), an Ig (e.g., hIg, mIg) constant region, a fragment of an Ig (e.g., hIg, mIg) constant region, an Ig (e.g., hIg, mIg) Fc region, human transferrin, a human transferrin binding moiety (e.g., small molecule, lipid, protein, peptide, etc.), human serum albumin (HSA), a fragment of HSA, an HSA binding moiety (e.g., small molecule, lipid, protein, peptide, etc.) (e.g., an antibody, a Streptococcal protein G (see, e.g., Mester 2021), polyethylene glycol (PEG) (and polymers thereof) (e.g., pegylation), lipids, small molecules, carbohydrates (e.g., glycosylation, polysialic acid (polysialylation), hydroxyethyl starch (HES) (HESylation), heparosan (HEPylation)).

Further, exemplary half-life extension polypeptides include, but are not limited to, an Ig, a fragment of an Ig, one or more Ig heavy chain constant region, a fragment of an Ig constant region, an Ig Fc region, a hIg, a fragment of a hIg, one or more hIg heavy chain constant region, a fragment of a hIg constant region, a hIg Fc region, a mIg, a fragment of a mIg, one or more mIg heavy chain constant region, a fragment of a mIg constant region, a mIg Fc region, human transferrin, a fragment of human transferrin, a human transferrin binding protein (e.g., an antibody) HSA, and HSA binding proteins (e.g., an antibody, a Streptococcal protein G). In some embodiments, the half-life extension polypeptide comprises an Ig Fc region (e.g., hIg Fc region). In some embodiments, the Ig (e.g., hIg, mIg) Fc region of a fusion protein described herein comprises one or more amino acid variation (e.g., relative to a reference Ig (e.g., hIg, mIg) Fc region (e.g., a wild-type Ig (e.g., hIg, mIg) Fc region)) that enhances serum half-life of the fusion protein (e.g., relative to a reference Ig (e.g., hIg, mIg) Fc region (e.g., a wild-type Ig (e.g., hIg, mIg) Fc region)). See, e.g., §§ 5.4.3, 5.4.4.

In some embodiments, half-life extension is mediated through one or more of lipidation, glycosylation, polysialylation, HESylation, HEPylation, and/or pegylation. In some embodiments, half-life extension is mediated through one or more of lipidation, HESylation, HEPylation, and/or pegylation. In some embodiments, half-life extension is mediated through glycosylation. In some embodiments, half-life extension is mediated through polysialylation.

In some embodiments, the half-life extension moiety comprises one or more lipids. See, e.g., Bech 2018. In some embodiments, the half-life extension moiety comprises one or more post translational modifications (e.g., glycosylation, polysialylation, etc.).

In some embodiments, the half-life extension moiety (e.g., protein) is altered (e.g., compared to a reference half-life extension moiety (e.g., protein)) to further enhance half-life of the fusion protein or conjugate. Various alterations to known half-life extension moieties (e.g., proteins) are known in the art. See, e.g., Ko 2021, Bech 2018, Mester 2021, and Kontermann 2011. Modifications include, e.g., amino acid variations (e.g., substitutions, additions, deletions) and post translational modifications (e.g., altered lipidation, glycosylation, polysialylation, HESylation, HEPylation, pegylation, etc.).

Fusion proteins described herein can be evaluated for their pharmacokinetic properties utilizing standard in vivo methods known in the art. See, e.g., Avery, Lindsay B et al. “Utility of a human FcRn transgenic mouse model in drug discovery for early assessment and prediction of human pharmacokinetics of monoclonal antibodies.” mAbs vol. 8,6 (2016): 1064-78. doi:10.1080/19420862.2016.1193660; Conner, Christopher M et al. “A precisely humanized FCRN transgenic mouse for preclinical pharmacokinetics studies.” Biochemical pharmacology vol. 210 (2023): 115470. doi:10.1016/j.bcp.2023.115470; and Kathryn Ball et al., PK and Biodistribution of Therapeutic Proteins, Drug Metabolism and Disposition Jun. 1, 2022, 50 (6) 858-866; DOI: https://doi.org/10.1124/dmd.121.000463 (hereinafter “Ball 2022”), the entire contents of each of which are incorporated herein by reference for all purposes.

### 5.4 Ig Fusion Proteins

In some embodiments, a fusion protein described herein comprises an immunoglobulin (e.g., antibody, Ig Fc region, etc.). In some embodiments, the immunoglobulin is an antibody (specifically binds a target antigen). In some embodiments, the immunoglobulin does not specifically bind a target antigen (e.g., one or more Ig constant region, and Ig Fc region, etc.). In some embodiments, an antibody described herein comprises one or more Ig constant regions. As such, where Ig constant regions are described herein (e.g., § 5.4.2), the embodiments relate to both fusion proteins comprising an antibody comprising the Ig constant region (as described in e.g., § 5.4.1) as well as fusion proteins comprising an immunoglobulin that does not bind a target antigen (e.g., comprising an Ig Fc region, e.g., as described in § 5.4.2).

### 5.4.1 Antibody Fusion Proteins

In some embodiments, a fusion protein described herein comprises an antibody. The antibody can act to further target the fusion protein e.g., to a specified cell type expressing a specific cell surface protein. The antibody can further be an antagonist or agonistic.

In some embodiments, the heterologous protein comprises an antibody. In some embodiments, the antibody can act to further target the immunoreceptor inhibitory protein e.g., to a specified cell or tissue type expressing a specific protein (e.g., cell surface protein). Exemplary antibodies include, full-length antibodies, scFvs, Fabs, single domain antibodies (e.g., VHHs), scFv-Fcs, Fab-Fcs, and single domain antibody-Fcs (e.g., VHH-Fcs). In some embodiments, the antibody comprises a full-length antibody. In some embodiments, the antibody comprises a scFv. In some embodiments, the antibody comprises a Fab. In some embodiments, the antibody comprises a single domain antibody. In some embodiments, the antibody comprises a VHH. In some embodiments, the antibody comprises an Fc region.

In some embodiments, the fusion protein comprises one or more antibody mimetic (e.g., described herein). For example, one or more 10th type III domain of fibronectin (e.g., AdNectins®) or designed ankyrin repeat proteins (e.g., DARPins®).

### 5.4.2 Ig Constant Region (e.g., Ig Fe) Fusion Proteins

In some embodiments, a fusion protein described herein comprises one or more Ig constant region. As described above the one or more Ig constant region can be part of an antibody or an Ig that does not specifically bind a target antigen (e.g., an Ig Fc region).

In some embodiments, a fusion protein described herein comprises one or more Ig heavy chain constant regions (e.g., a CH2 region, a CH3 region, a hinge region, an Fc region (e.g., in some embodiments, preferably an Fc region). In some embodiments, the Ig is an IgG. In some embodiments, the IgG is IgG1, IgG2, IgG3, or IgG4 (e.g., in some embodiments preferably an IgG4).

In some embodiments, the fusion protein comprises an IgG CH2 region and an IgG CH3 region. In some embodiments, the fusion protein comprises a partial IgG hinge region, IgG CH2 region, and IgG CH3 region. In some embodiments, the fusion protein comprises an IgG hinge region, IgG CH2 region, and IgG CH3 region. In some embodiments, the fusion protein comprises an IgG1 CH2 region and an IgG1 CH3 region. In some embodiments, the fusion protein comprises a partial IgG1 hinge region, IgG1 CH2 region, and IgG1 CH3 region. In some embodiments, the fusion protein comprises an IgG1 hinge region, IgG1 CH2 region, and IgG1 CH3 region. In some embodiments, the fusion protein comprises an IgG4 CH2 region and an IgG4 CH3 region. In some embodiments, the fusion protein comprises a partial IgG4 hinge region, IgG4 CH2 region, and IgG4 CH3 region. In some embodiments, the fusion protein comprises an IgG4 hinge region, IgG4 CH2 region, and IgG4 CH3 region.

In some embodiments, the fusion protein comprises a protein consisting of an IgG CH2 region and an IgG CH3 region. In some embodiments, the fusion protein comprises a protein consisting of a partial IgG hinge region, IgG CH2 region, and IgG CH3 region. In some embodiments, the fusion protein comprises a protein consisting of an IgG hinge region, IgG CH2 region, and IgG CH3 region. In some embodiments, the fusion protein comprises a protein consisting of an IgG1 CH2 region and an IgG1 CH3 region. In some embodiments, the fusion protein comprises a protein consisting of a partial IgG1 hinge region, IgG1 CH2 region, and IgG1 CH3 region. In some embodiments, the fusion protein comprises a protein consisting of an IgG1 hinge region, IgG1 CH2 region, and IgG1 CH3 region. In some embodiments, the fusion protein comprises a protein consisting of an IgG4 CH2 region and an IgG4 CH3 region. In some embodiments, the fusion protein comprises a protein consisting of a partial IgG4 hinge region, IgG4 CH2 region, and IgG4 CH3 region. In some embodiments, the fusion protein comprises a protein consisting of an IgG4 hinge region, IgG4 CH2 region, and IgG4 CH3 region.

In some embodiments, the fusion protein comprises an Ig Fc region. In some embodiments, the Ig Fc region comprises at least a portion of a hinge region, a CH2 region, and a CH3 region. In some embodiments, the Ig Fc region comprises a hinge region, a CH2 region, and a CH3 region. In some embodiments, the Ig Fc region comprises at least a portion of an IgG hinge region, an IgG CH2 region, and an IgG CH3 region. In some embodiments, the Ig Fc region comprises an IgG hinge region, an IgG CH2 region, and an IgG CH3 region. In some embodiments, the Ig Fc region comprises at least a portion of an IgG1 hinge region, an IgG1 CH2 region, and an IgG1 CH3 region. In some embodiments, the Ig Fc region comprises an IgG1 hinge region, an IgG1 CH2 region, and an IgG1 CH3 region. In some embodiments, the Ig Fc region comprises at least a portion of an IgG4 hinge region, an IgG4 CH2 region, and an IgG4 CH3 region. In some embodiments, the Ig Fc region comprises an IgG4 hinge region, an IgG4 CH2 region, and an IgG4 CH3 region.

In some embodiments, the Ig Fc region consists of at least a portion of a hinge region, a CH2 region, and a CH3 region. In some embodiments, the Ig Fc region consists of a hinge region, a CH2 region, and a CH3 region. In some embodiments, the Ig Fc region consists of at least a portion of an IgG hinge region, an IgG CH2 region, and an IgG CH3 region. In some embodiments, the Ig Fc region consists of an IgG hinge region, an IgG CH2 region, and an IgG CH3 region. In some embodiments, the Ig Fc region consists of at least a portion of an IgG1 hinge region, an IgG1 CH2 region, and an IgG1 CH3 region. In some embodiments, the Ig Fc region consists of an IgG1 hinge region, an IgG1 CH2 region, and an IgG1 CH3 region. In some embodiments, the Ig Fc region consists of at least a portion of an IgG4 hinge region, an IgG4 CH2 region, and an IgG4 CH3 region. In some embodiments, the Ig Fc region consists of an IgG4 hinge region, an IgG4 CH2 region, and an IgG4 CH3 region.

In some embodiments, the fusion protein comprises one or more hIg heavy chain constant regions (e.g., a CH2 region, a CH3 region, a hinge region, an Fc region). In some embodiments, the hIg is a human IgG (hIgG). In some embodiments, the hIgG is hIgG1, IgG2, IgG3, or IgG4. In some embodiments, the hIgG is IgG1 or IgG4. In some embodiments, the hIgG is hIgG1. In some embodiments, the hIgG is hIgG4.

In some embodiments, the fusion protein comprises a hIgG CH2 region and a hIgG CH3 region. In some embodiments, the fusion protein comprises a partial hIgG hinge region, hIgG CH2 region, and hIgG CH3 region. In some embodiments, the fusion protein comprises a hIgG hinge region, hIgG CH2 region, and hIgG CH3 region. In some embodiments, the fusion protein comprises a hIgG1 CH2 region and a hIgG1 CH3 region. In some embodiments, the fusion protein comprises a partial hIgG1 hinge region, hIgG1 CH2 region, and hIgG1 CH3 region. In some embodiments, the fusion protein comprises a hIgG1 hinge region, hIgG1 CH2 region, and hIgG1 CH3 region. In some embodiments, the fusion protein comprises a hIgG4 CH2 region and a hIgG4 CH3 region. In some embodiments, the fusion protein comprises a partial hIgG4 hinge region, hIgG4 CH2 region, and hIgG4 CH3 region. In some embodiments, the fusion protein comprises a hIgG4 hinge region, hIgG4 CH2 region, and hIgG4 CH3 region.

In some embodiments, the fusion protein comprises a protein that consists of a hIgG CH2 region and a hIgG CH3 region. In some embodiments, the fusion protein comprises a protein that consists of a partial hIgG hinge region, hIgG CH2 region, and hIgG CH3 region. In some embodiments, the fusion protein comprises a protein that consists of a hIgG hinge region, hIgG CH2 region, and hIgG CH3 region. In some embodiments, the fusion protein comprises a protein that consists of a hIgG1 CH2 region and a hIgG1 CH3 region. In some embodiments, the fusion protein comprises a protein that consists of a partial hIgG1 hinge region, hIgG1 CH2 region, and hIgG1 CH3 region. In some embodiments, the fusion protein comprises a protein that consists of a hIgG1 hinge region, hIgG1 CH2 region, and hIgG1 CH3 region. In some embodiments, the fusion protein comprises a protein that consists of a hIgG4 CH2 region and a hIgG4 CH3 region. In some embodiments, the fusion protein comprises a protein that consists of a partial hIgG4 hinge region, hIgG4 CH2 region, and hIgG4 CH3 region. In some embodiments, the fusion protein comprises a protein that consists of a hIgG4 hinge region, hIgG4 CH2 region, and hIgG4 CH3 region.

In some embodiments, the fusion protein comprises a hIg Fc region. In some embodiments, the hIg Fc region comprises at least a portion of a hinge region, a CH2 region, and a CH3 region. In some embodiments, the hIg Fc region comprises a hinge region, a CH2 region, and a CH3 region. In some embodiments, the hIg Fc region comprises at least a portion of a hIgG hinge region, a hIgG CH2 region, and a hIgG CH3 region. In some embodiments, the hIg Fc region comprises a hIgG hinge region, a hIgG CH2 region, and a hIgG CH3 region. In some embodiments, the hIg Fc region comprises at least a portion of a hIgG1 hinge region, a hIgG1 CH2 region, and a hIgG1 CH3 region. In some embodiments, the hIg Fc region comprises a hIgG1 hinge region, a hIgG1 CH2 region, and a hIgG1 CH3 region. In some embodiments, the hIg Fc region comprises at least a portion of a hIgG4 hinge region, a hIgG4 CH2 region, and a hIgG4 CH3 region. In some embodiments, the hIg Fc region comprises a hIgG4 hinge region, a hIgG4 CH2 region, and a hIgG4 CH3 region.

In some embodiments, the fusion protein consists of a hIg Fc region. In some embodiments, the hIg Fc region consists of at least a portion of a hinge region, a CH2 region, and a CH3 region. In some embodiments, the hIg Fc region consists of a hinge region, a CH2 region, and a CH3 region. In some embodiments, the hIg Fc region consists of at least a portion of a hIgG hinge region, a hIgG CH2 region, and a hIgG CH3 region. In some embodiments, the hIg Fc region consists of a hIgG hinge region, a hIgG CH2 region, and a hIgG CH3 region. In some embodiments, the hIg Fc region consists of at least a portion of a hIgG1 hinge region, a hIgG1 CH2 region, and a hIgG1 CH3 region. In some embodiments, the hIg Fc region consists of a hIgG1 hinge region, a hIgG1 CH2 region, and a hIgG1 CH3 region. In some embodiments, the hIg Fc region consists of at least a portion of a hIgG4 hinge region, a hIgG4 CH2 region, and a hIgG4 CH3 region. In some embodiments, the hIg Fc region consists of a hIgG4 hinge region, a hIgG4 CH2 region, and a hIgG4 CH3 region.

In some embodiments, the fusion protein comprises one or more hIg light chain constant region (e.g., κCL or λCL) (e.g., as part of an antibody (e.g., a full-length antibody, a Fab)).

The amino acid sequence of exemplary reference hIgG1 and hIgG4 heavy chain constant regions and hIg light chain constant regions, which can be incorporated in one or more of the embodiments described herein (e.g., fusion proteins described herein), is provided in Table 8.

TABLE 8
The Amino Acid Sequence of Exemplary hIg heavy chain constant region
components and hIg light chain constant regions.

| | | SEQ ID |
|---|---|---|
| Description | Amino Acid Sequence | NO |
| hIgG1 CH1 Region | ASTKGPSVFPLAPSSKSTSGGTAALGCLVKDYFPEPVTVSW | 632 |
| | NSGALTSGVHTFPAVLQSSGLYSLSSVVTVPSSSLGTQTYI | |
| | CNVNHKPSNTKVDKKV | |
| hIgG1 Hinge Region | EPKSCDKTHTCP | 633 |
| hIgG1 CH2 Region | PCPAPELLGGPSVFLFPPKPKDTLMISRTPEVTCVVVDVSH | 634 |
| | EDPEVKFNWYVDGVEVHNAKTKPREEQYNSTYRVVSVLTVL | |
| | HQDWLNGKEYKCKVSNKALPAPIEKTISKAK | |
| hIgG1 CH3 Region | GQPREPQVYTLPPSRDELTKNQVSLTCLVKGFYPSDIAVEW | 635 |
| With C-terminal Lysine | ESNGQPENNYKTTPPVLDSDGSFFLYSKLTVDKSRWQQGNV | |
| | FSCSVMHEALHNHYTQKSLSLSPGK | |
| hIgG1 CH3 Region | GQPREPQVYTLPPSRDELTKNQVSLTCLVKGFYPSDIAVEW | 636 |
| Without C-terminal | ESNGQPENNYKTTPPVLDSDGSFFLYSKLTVDKSRWQQGNV | |
| Lysine | FSCSVMHEALHNHYTQKSLSLSPG | |
| hIgG1 CH2 Region + | PCPAPELLGGPSVFLFPPKPKDTLMISRTPEVTCVVVDVSH | 637 |
| CH3 Region | EDPEVKFNWYVDGVEVHNAKTKPREEQYNSTYRVVSVLTVL | |
| With C-terminal Lysine | HQDWLNGKEYKCKVSNKALPAPIEKTISKAKGQPREPQVYT | |
| | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPGK | |
| hIgG1 CH2 Region + | PCPAPELLGGPSVFLFPPKPKDTLMISRTPEVTCVVVDVSH | 638 |
| CH3 Region | EDPEVKFNWYVDGVEVHNAKTKPREEQYNSTYRVVSVLTVL | |
| Without C-terminal | HQDWLNGKEYKCKVSNKALPAPIEKTISKAKGQPREPQVYT | |
| Lysine | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| hIgG1 Partial Hinge | TCPPCPAPELLGGPSVFLFPPKPKDTLMISRTPEVTCVVVD | 639 |
| Region + CH2 Region + | VSHEDPEVKFNWYVDGVEVHNAKTKPREEQYNSTYRVVSVL | |
| CH3 Region | TVLHQDWLNGKEYKCKVSNKALPAPIEKTISKAKGQPREPQ | |
| With C-terminal Lysine | VYTLPPSRDELTKNQVSLTCLVKGFYPSDIAVEWESNGQPE | |
| | NNYKTTPPVLDSDGSFFLYSKLTVDKSRWQQGNVFSCSVMH | |
| | EALHNHYTQKSLSLSPGK | |
| hIgG1 Partial Hinge | TCPPCPAPELLGGPSVFLFPPKPKDTLMISRTPEVTCVVVD | 640 |
| Region + CH2 Region + | VSHEDPEVKFNWYVDGVEVHNAKTKPREEQYNSTYRVVSVL | |
| CH3 Region | TVLHQDWLNGKEYKCKVSNKALPAPIEKTISKAKGQPREPQ | |
| Without C-terminal | VYTLPPSRDELTKNQVSLTCLVKGFYPSDIAVEWESNGQPE | |
| Lysine | NNYKTTPPVLDSDGSFFLYSKLTVDKSRWQQGNVFSCSVMH | |
| | EALHNHYTQKSLSLSPG | |
| hIgG1 Partial Hinge | DKTHTCPPCPAPELLGGPSVFLFPPKPKDTLMISRTPEVTC | 641 |
| Region + CH2 Region + | VVVDVSHEDPEVKFNWYVDGVEVHNAKTKPREEQYNSTYRV | |
| CH3 Region | VSVLTVLHQDWLNGKEYKCKVSNKALPAPIEKTISKAKGQP | |
| With C-terminal Lysine | REPQVYTLPPSRDELTKNQVSLTCLVKGFYPSDIAVEWESN | |
| | GQPENNYKTTPPVLDSDGSFFLYSKLTVDKSRWQQGNVFSC | |
| | SVMHEALHNHYTQKSLSLSPGK | |
| hIgG1 Partial Hinge | DKTHTCPPCPAPELLGGPSVFLFPPKPKDTLMISRTPEVTC | 642 |
| Region + CH2 Region + | VVVDVSHEDPEVKFNWYVDGVEVHNAKTKPREEQYNSTYRV | |
| CH3 Region | VSVLTVLHQDWLNGKEYKCKVSNKALPAPIEKTISKAKGQP | |
| Without C-terminal | REPQVYTLPPSRDELTKNQVSLTCLVKGFYPSDIAVEWESN | |
| Lysine | GQPENNYKTTPPVLDSDGSFFLYSKLTVDKSRWQQGNVFSC | |
| | SVMHEALHNHYTQKSLSLSPG | |
| hIgG1 Hinge Region + | EPKSCDKTHTCPPCPAPELLGGPSVFLFPPKPKDTLMISRT | 643 |
| CH2 Region + CH3 | PEVTCVVVDVSHEDPEVKFNWYVDGVEVHNAKTKPREEQYN | |
| Region | STYRVVSVLTVLHQDWLNGKEYKCKVSNKALPAPIEKTISK | |
| With C-terminal Lysine | AKGQPREPQVYTLPPSRDELTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| hIgG1 Hinge Region + | EPKSCDKTHTCPPCPAPELLGGPSVFLFPPKPKDTLMISRT | 644 |
| CH2 Region + CH3 | PEVTCVVVDVSHEDPEVKFNWYVDGVEVHNAKTKPREEQYN | |
| Region | STYRVVSVLTVLHQDWLNGKEYKCKVSNKALPAPIEKTISK | |
| Without C-terminal | AKGQPREPQVYTLPPSRDELTKNQVSLTCLVKGFYPSDIAV | |
| Lysine | EWESNGQPENNYKTTPPVLDSDGSFFLYSKLTVDKSRWQQG | |
| | NVFSCSVMHEALHNHYTQKSLSLSPG | |
| hIgG1 CH1+ Hinge | ASTKGPSVFPLAPSSKSTSGGTAALGCLVKDYFPEPVTVSW | 645 |
| Region + CH2 Region + | NSGALTSGVHTFPAVLQSSGLYSLSSVVTVPSSSLGTQTYI | |
| CH3 Region | CNVNHKPSNTKVDKKVEPKSCDKTHTCPPCPAPELLGGPSV | |
| With C-terminal Lysine | FLFPPKPKDTLMISRTPEVTCVVVDVSHEDPEVKFNWYVDG | |
| | VEVHNAKTKPREEQYNSTYRVVSVLTVLHQDWLNGKEYKCK | |
| | VSNKALPAPIEKTISKAKGQPREPQVYTLPPSRDELTKNQV | |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | GK | |
| hIgG1 CH1 + Hinge | ASTKGPSVFPLAPSSKSTSGGTAALGCLVKDYFPEPVTVSW | 646 |
| Region + CH2 Region + | NSGALTSGVHTFPAVLQSSGLYSLSSVVTVPSSSLGTQTYI | |
| CH3 Region | CNVNHKPSNTKVDKKVEPKSCDKTHTCPPCPAPELLGGPSV | |
| Without C-terminal | FLFPPKPKDTLMISRTPEVTCVVVDVSHEDPEVKFNWYVDG | |
| Lysine | VEVHNAKTKPREEQYNSTYRVVSVLTVLHQDWLNGKEYKCK | |
| | VSNKALPAPIEKTISKAKGQPREPQVYTLPPSRDELTKNQV | |
| | SLTCLVKGFYPSDIAVEWESNGQPENNYKTTPPVLDSDGSF | |
| | FLYSKLTVDKSRWQQGNVFSCSVMHEALHNHYTQKSLSLSP | |
| | G | |
| hIgG4 CH1 Region | ASTKGPSVFPLAPCSRSTSESTAALGCLVKDYFPEPVTVSW | 647 |
| | NSGALTSGVHTFPAVLQSSGLYSLSSVVTVPSSSLGTKTYT | |
| | CNVDHKPSNTKVDKRV | |
| hIgG4 Hinge Region | ESKYGPPCPSCP | 648 |
| hIgG4 Hinge Region | AESKYGPPCPSCP | 649 |
| (Variant) | | |
| hIgG4 CH2 Region | APEFLGGPSVFLFPPKPKDTLMISRTPEVTCVVVDVSQEDP | 650 |
| | EVQFNWYVDGVEVHNAKTKPREEQFNSTYRVVSVLTVLHQD | |
| | WLNGKEYKCKVSNKGLPSSIEKTISKAK | |
| hIgG4 CH3 Region | GQPREPQVYTLPPSQEEMTKNQVSLTCLVKGFYPSDIAVEW | 651 |
| With C-terminal Lysine | ESNGQPENNYKTTPPVLDSDGSFFLYSRLTVDKSRWQEGNV | |
| | FSCSVMHEALHNHYTQKSLSLSLGK | |
| hIgG4 CH3 Region | GQPREPQVYTLPPSQEEMTKNQVSLTCLVKGFYPSDIAVEW | 652 |
| Without C-terminal | ESNGQPENNYKTTPPVLDSDGSFFLYSRLTVDKSRWQEGNV | |
| Lysine | FSCSVMHEALHNHYTQKSLSLSLG | |
| hIgG4 CH2 Region + | APEFLGGPSVFLFPPKPKDTLMISRTPEVTCVVVDVSQEDP | 653 |
| CH3 Region | EVQFNWYVDGVEVHNAKTKPREEQFNSTYRVVSVLTVLHQD | |
| With C-terminal Lysine | WLNGKEYKCKVSNKGLPSSIEKTISKAKGQPREPQVYTLPP | |
| | SQEEMTKNQVSLTCLVKGFYPSDIAVEWESNGQPENNYKTT | |
| | PPVLDSDGSFFLYSRLTVDKSRWQEGNVFSCSVMHEALHNH | |
| | YTQKSLSLSLGK | |
| hIgG4 CH2 Region + | APEFLGGPSVFLFPPKPKDTLMISRTPEVTCVVVDVSQEDP | 654 |
| CH3 Region | EVQFNWYVDGVEVHNAKTKPREEQFNSTYRVVSVLTVLHQD | |
| Without C-terminal | WLNGKEYKCKVSNKGLPSSIEKTISKAKGQPREPQVYTLPP | |
| Lysine | SQEEMTKNQVSLTCLVKGFYPSDIAVEWESNGQPENNYKTT | |
| | PPVLDSDGSFFLYSRLTVDKSRWQEGNVFSCSVMHEALHNH | |
| | YTQKSLSLSLG | |
| hIgG4 Partial Hinge | PCPSCPAPEFLGGPSVFLFPPKPKDTLMISRTPEVTCVVVD | 655 |
| Region + CH2 Region + | VSQEDPEVQFNWYVDGVEVHNAKTKPREEQFNSTYRVVSVL | |
| CH3 Region | TVLHQDWLNGKEYKCKVSNKGLPSSIEKTISKAKGQPREPQ | |
| With C-terminal Lysine | VYTLPPSQEEMTKNQVSLTCLVKGFYPSDIAVEWESNGQPE | |
| | NNYKTTPPVLDSDGSFFLYSRLTVDKSRWQEGNVFSCSVMH | |
| | EALHNHYTQKSLSLSLGK | |
| hIgG4 Partial Hinge | PCPSCPAPEFLGGPSVFLFPPKPKDTLMISRTPEVTCVVVD | 656 |
| Region + CH2 Region + | VSQEDPEVQFNWYVDGVEVHNAKTKPREEQFNSTYRVVSVL | |
| CH3 Region | TVLHQDWLNGKEYKCKVSNKGLPSSIEKTISKAKGQPREPQ | |
| Without C-terminal | VYTLPPSQEEMTKNQVSLTCLVKGFYPSDIAVEWESNGQPE | |
| Lysine | NNYKTTPPVLDSDGSFFLYSRLTVDKSRWQEGNVFSCSVMH | |
| | EALHNHYTQKSLSLSLG | |
| hIgG4 Hinge Region + | ESKYGPPCPSCPAPEFLGGPSVFLFPPKPKDTLMISRTPEV | 657 |
| CH2 Region + CH3 | TCVVVDVSQEDPEVQFNWYVDGVEVHNAKTKPREEQFNSTY | |
| Region | RVVSVLTVLHQDWLNGKEYKCKVSNKGLPSSIEKTISKAKG | |
| With C-terminal Lysine | QPREPQVYTLPPSQEEMTKNQVSLTCLVKGFYPSDIAVEWE | |
| | SNGQPENNYKTTPPVLDSDGSFFLYSRLTVDKSRWQEGNVF | |
| | SCSVMHEALHNHYTQKSLSLSLGK | |
| hIgG4 Hinge Region + | ESKYGPPCPSCPAPEFLGGPSVFLFPPKPKDTLMISRTPEV | 658 |
| CH2 Region + CH3 | TCVVVDVSQEDPEVQFNWYVDGVEVHNAKTKPREEQFNSTY | |
| Region | RVVSVLTVLHQDWLNGKEYKCKVSNKGLPSSIEKTISKAKG | |
| Without C-terminal | QPREPQVYTLPPSQEEMTKNQVSLTCLVKGFYPSDIAVEWE | |
| Lysine | SNGQPENNYKTTPPVLDSDGSFFLYSRLTVDKSRWQEGNVF | |
| | SCSVMHEALHNHYTQKSLSLSLG | |
| hIgG4 Hinge Region + | AESKYGPPCPSCPAPEFLGGPSVFLFPPKPKDTLMISRTPE | 659 |
| CH2 Region + CH3 | VTCVVVDVSQEDPEVQFNWYVDGVEVHNAKTKPREEQFNST | |
| Region | YRVVSVLTVLHQDWLNGKEYKCKVSNKGLPSSIEKTISKAK | |
| (Variant) | GQPREPQVYTLPPSQEEMTKNQVSLTCLVKGFYPSDIAVEW | |
| With C-terminal Lysine | ESNGQPENNYKTTPPVLDSDGSFFLYSRLTVDKSRWQEGNV | |
| | FSCSVMHEALHNHYTQKSLSLSLGK | |
| hIgG4 Hinge Region + | AESKYGPPCPSCPAPEFLGGPSVFLFPPKPKDTLMISRTPE | 660 |
| CH2 Region + CH3 | VTCVVVDVSQEDPEVQFNWYVDGVEVHNAKTKPREEQFNST | |
| Region | YRVVSVLTVLHQDWLNGKEYKCKVSNKGLPSSIEKTISKAK | |
| (Variant) | GQPREPQVYTLPPSQEEMTKNQVSLTCLVKGFYPSDIAVEW | |
| Without C-terminal | ESNGQPENNYKTTPPVLDSDGSFFLYSRLTVDKSRWQEGNV | |
| Lysine | FSCSVMHEALHNHYTQKSLSLSLG | |
| hIgG4 CH1 + Hinge | ASTKGPSVFPLAPCSRSTSESTAALGCLVKDYFPEPVTVSW | 661 |
| Region + CH2 Region + | NSGALTSGVHTFPAVLQSSGLYSLSSVVTVPSSSLGTKTYT | |
| CH3 Region | CNVDHKPSNTKVDKRVESKYGPPCPSCPAPEFLGGPSVFLF | |
| With C-terminal Lysine | PPKPKDTLMISRTPEVTCVVVDVSQEDPEVQFNWYVDGVEV | |
| | HNAKTKPREEQFNSTYRVVSVLTVLHQDWLNGKEYKCKVSN | |
| | KGLPSSIEKTISKAKGQPREPQVYTLPPSQEEMTKNQVSLT | |
| | CLVKGFYPSDIAVEWESNGQPENNYKTTPPVLDSDGSFFLY | |
| | SRLTVDKSRWQEGNVFSCSVMHEALHNHYTQKSLSLSLGK | |
| hIgG4 CH1 + Hinge | ASTKGPSVFPLAPCSRSTSESTAALGCLVKDYFPEPVTVSW | 662 |
| Region + CH2 Region + | NSGALTSGVHTFPAVLQSSGLYSLSSVVTVPSSSLGTKTYT | |
| CH3 Region | CNVDHKPSNTKVDKRVESKYGPPCPSCPAPEFLGGPSVFLF | |
| Without C-terminal | PPKPKDTLMISRTPEVTCVVVDVSQEDPEVQFNWYVDGVEV | |
| Lysine | HNAKTKPREEQFNSTYRVVSVLTVLHQDWLNGKEYKCKVSN | |
| | KGLPSSIEKTISKAKGQPREPQVYTLPPSQEEMTKNQVSLT | |
| | CLVKGFYPSDIAVEWESNGQPENNYKTTPPVLDSDGSFFLY | |
| | SRLTVDKSRWQEGNVFSCSVMHEALHNHYTQKSLSLSLG | |
| Ig light chain kappa | RTVAAPSVFIFPPSDEQLKSGTASVVCLLNNFYPREAKVQW | 663 |
| constant region (κCL) | KVDNALQSGNSQESVTEQDSKDSTYSLSSTLTLSKADYEKH | |
| | KVYACEVTHQGLSSPVTKSFNRGEC | |
| Ig light chain kappa | GQPKANPTVTLFPPSSEELQANKATLVCLISDFYPGAVTVA | 664 |
| constant region (λCL) | WKADGSPVKAGVETTKPSKQSNNKYAASSYLSLTPEQWKSH | |
| | RSYSCQVTHEGSTVEKTVAPTECS | |

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 8. In some embodiments, a fusion protein described herein comprises a protein (or a functional fragment, functional variant, or functional fragment/variant thereof) comprising an amino acid sequence at least 85% identical to the amino acid sequence of a protein set forth in Table 8. In some embodiments, a fusion protein described herein comprises a protein ((or a functional fragment, functional variant, or functional fragment/variant thereof) comprising an amino acid sequence at least 90% identical to the amino acid sequence of a protein set forth in Table 8. In some embodiments, a fusion protein described herein comprises a protein (or a functional fragment, functional variant, or functional fragment/variant thereof) comprising an amino acid sequence at least 95% identical to the amino acid sequence of a protein set forth in Table 8. In some embodiments, a fusion protein described herein comprises a protein (or a functional fragment, functional variant, or functional fragment/variant thereof) comprising an amino acid sequence at least 99% identical to the amino acid sequence of a protein set forth in Table 8.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 8. In some embodiments, a fusion protein described herein comprises a protein (or a functional fragment, functional variant, or functional fragment/variant thereof) comprising an amino acid sequence 85% identical to the amino acid sequence of a protein set forth in Table 8. In some embodiments, a fusion protein described herein comprises a protein ((or a functional fragment, functional variant, or functional fragment/variant thereof) comprising an amino acid sequence 90% identical to the amino acid sequence of a protein set forth in Table 8. In some embodiments, a fusion protein described herein comprises a protein (or a functional fragment, functional variant, or functional fragment/variant thereof) comprising an amino acid sequence 95% identical to the amino acid sequence of a protein set forth in Table 8. In some embodiments, a fusion protein described herein comprises a protein (or a functional fragment, functional variant, or functional fragment/variant thereof) comprising an amino acid sequence 99% identical to the amino acid sequence of a protein set forth in Table 8. In some embodiments, a fusion protein described herein comprises a protein (or a functional fragment, functional variant, or functional fragment/variant thereof) comprising an amino acid sequence 100% identical to the amino acid sequence of a protein set forth in Table 8.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 8. In some embodiments, a fusion protein described herein comprises a protein (or a functional fragment, functional variant, or functional fragment/variant thereof) consisting of an amino acid sequence at least 85% identical to the amino acid sequence of a protein set forth in Table 8. In some embodiments, a fusion protein described herein comprises a protein (or a functional fragment, functional variant, or functional fragment/variant thereof) consisting of an amino acid sequence at least 90% identical to the amino acid sequence of a protein set forth in Table 8. In some embodiments, a fusion protein described herein comprises a protein (or a functional fragment, functional variant, or functional fragment/variant thereof) consisting of an amino acid sequence at least 95% identical to the amino acid sequence of a protein set forth in Table 8. In some embodiments, a fusion protein described herein comprises a protein (or a functional fragment, functional variant, or functional fragment/variant thereof) consisting of an amino acid sequence at least 99% identical to the amino acid sequence of a protein set forth in Table 8.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 8. In some embodiments, a fusion protein described herein comprises a protein (or a functional fragment, functional variant, or functional fragment/variant thereof) consisting of an amino acid sequence 85% identical to the amino acid sequence of a protein set forth in Table 8. In some embodiments, a fusion protein described herein comprises a protein (or a functional fragment, functional variant, or functional fragment/variant thereof) consisting of an amino acid sequence 90% identical to the amino acid sequence of a protein set forth in Table 8. In some embodiments, a fusion protein described herein comprises a protein (or a functional fragment, functional variant, or functional fragment/variant thereof) consisting of an amino acid sequence 95% identical to the amino acid sequence of a protein set forth in Table 8. In some embodiments, a fusion protein described herein comprises a protein (or a functional fragment, functional variant, or functional fragment/variant thereof) consisting of an amino acid sequence 99% identical to the amino acid sequence of a protein set forth in Table 8. In some embodiments, a fusion protein described herein comprises a protein (or a functional fragment, functional variant, or functional fragment/variant thereof) consisting of an amino acid sequence 100% identical to the amino acid sequence of a protein set forth in Table 8.

In some embodiments, a fusion protein described herein comprises a protein (or a functional fragment, functional variant, or functional fragment/variant thereof) comprising the amino acid sequence of a protein set forth in Table 8, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 8, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 8, and further consisting of at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 8, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 8, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 8, and further comprising or no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence of a protein set forth in Table 8, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 8, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 8, and further consisting of at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 8, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 8, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 8, and further comprising or no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence at least 85% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence at least 90% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence at least 95% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence at least 99% identical to the amino acid sequence set forth in any one of SEQ ID NOS: 632-664.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence 85% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence 90% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence 95% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence 99% identical to the amino acid sequence set forth in any one of SEQ ID NOS: 632-664. In some embodiments, a fusion protein described herein comprises a protein (or a functional fragment, functional variant, or functional fragment/variant thereof) comprising an amino acid sequence 100% identical to the amino acid sequence set forth in any one of SEQ ID NOS: 632-664.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence at least 85% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence at least 90% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence at least 95% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence at least 99% identical to the amino acid sequence set forth in any one of SEQ ID NOS: 632-664.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence 85% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence 90% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence 95% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence 99% identical to the amino acid sequence set forth in any one of SEQ ID NOS: 632-664. In some embodiments, a fusion protein described herein comprises a protein (or a functional fragment, functional variant, or functional fragment/variant thereof) consisting of an amino acid sequence 100% identical to the amino acid sequence set forth in any one of SEQ ID NOS: 632-664.

In some embodiments, a fusion protein described herein comprises a protein (or a functional fragment, functional variant, or functional fragment/variant thereof) comprising the amino acid sequence set forth in any one of SEQ ID NOS: 632-664, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 632-664, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 632-664, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 632-664, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 632-664, and further comprising or consists of no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence set forth in any one of SEQ ID NOS: 632-664, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 632-664, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 632-664, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 632-664, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 632-664, and further consisting of no more than 1, 2, 3, 4, 5, 6, 7, 8, 9, or 10 amino acid variations (e.g., substitutions, additions, deletions, etc.).

In some embodiments, a fusion protein described herein comprises a CH3 region (e.g., comprises an Fc region; a hinge region, CH2 region, and CH3 region, etc.), wherein the CH3 region lacks the C-terminal lysine (e.g., residue 232 of SEQ ID NO: 643, EU numbering according to Kabat; or e.g., residue 229 of SEQ ID NO: 657, EU numbering according to Kabat). In some embodiments, the CH3 region further lacks the C-terminal glycine (e.g., residue 231 of SEQ ID NO: 643, EU numbering according to Kabat; or e.g., residue 228 of SEQ ID NO: 657, EU numbering according to Kabat).

In some embodiments, the fusion protein comprises one or more mIg heavy chain constant regions (e.g., a CH2 region, a CH3 region, a hinge region, an Fc region). In some embodiments, the mIg is mIgG (mIgG). In some embodiments, the mIgG is mIgG1, mIgG2a, mIgG2c, mIgG2b, or mIgG3. In some embodiments, the mIgG is mIgG1 or mIgG2a. In some embodiments, the mIgG is mIgG1. In some embodiments, the mIgG is mIgG2a.

In some embodiments, the fusion protein comprises a mIgG CH2 region and a mIgG CH3 region. In some embodiments, the fusion protein comprises a partial mIgG hinge region, mIgG CH2 region, and mIgG CH3 region. In some embodiments, the fusion protein comprises a mIgG hinge region, mIgG CH2 region, and mIgG CH3 region. In some embodiments, the fusion protein comprises a mIgG1 CH2 region and a mIgG1 CH3 region. In some embodiments, the fusion protein comprises a partial mIgG1 hinge region, mIgG1 CH2 region, and mIgG1 CH3 region. In some embodiments, the fusion protein comprises a mIgG1 hinge region, mIgG1 CH2 region, and mIgG1 CH3 region. In some embodiments, the fusion protein comprises a mIgG2a CH2 region and a mIgG2a CH3 region. In some embodiments, the fusion protein comprises a partial mIgG2a hinge region, mIg2a CH2 region, and mIgG2a CH3 region. In some embodiments, the fusion protein comprises a mIgG2a hinge region, mIgG2a CH2 region, and mIgG2a CH3 region.

In some embodiments, the fusion protein comprises a mIg Fc region. In some embodiments, the mIg Fc region comprises at least a portion of a hinge region, a CH2 region, and a CH3 region. In some embodiments, the mIg Fc region comprises a hinge region, a CH2 region, and a CH3 region. In some embodiments, the mIg Fc region comprises at least a portion of a mIgG hinge region, a mIgG CH2 region, and a mIgG CH3 region. In some embodiments, the mIg Fc region comprises a mIgG hinge region, a mIgG CH2 region, and a mIgG CH3 region. In some embodiments, the mIg Fc region comprises at least a portion of a mIgG1 hinge region, a mIgG1 CH2 region, and a mIgG1 CH3 region. In some embodiments, the mIg Fc region comprises a mIgG1 hinge region, a mIgG1 CH2 region, and a mIgG1 CH3 region. In some embodiments, the mIg Fc region comprises at least a portion of a mIgG2a hinge region, a mIgG2a CH2 region, and a mIgG2a CH3 region. In some embodiments, the mIg Fc region comprises a mIgG2a hinge region, a mIgG2a CH2 region, and a mIgG2a CH3 region.

In some embodiments, the mIg Fc region consists of at least a portion of a hinge region, a CH2 region, and a CH3 region. In some embodiments, the mIg Fc region consists of a hinge region, a CH2 region, and a CH3 region. In some embodiments, the mIg Fc region consists of at least a portion of a mIgG hinge region, a mIgG CH2 region, and a mIgG CH3 region. In some embodiments, the mIg Fc region consists of a mIgG hinge region, a mIgG CH2 region, and a mIgG CH3 region. In some embodiments, the mIg Fc region consists of at least a portion of a mIgG1 hinge region, a mIgG1 CH2 region, and a mIgG1 CH3 region. In some embodiments, the mIg Fc region consists of a mIgG1 hinge region, a mIgG1 CH2 region, and a mIgG1 CH3 region. In some embodiments, the mIg Fc region consists of at least a portion of a mIgG2a hinge region, a mIgG2a CH2 region, and a mIgG2a CH3 region. In some embodiments, the mIg Fc region consists of a mIgG2a hinge region, a mIgG2a CH2 region, and a mIgG2a CH3 region.

In some embodiments, the fusion protein comprises one or more mIg light chain constant region (e.g., as part of an antibody (e.g., a full-length antibody, a Fab)).

The amino acid sequence of exemplary reference mIgG1 and mIgG2a heavy chain constant regions and light chain constant regions, which can be incorporated in one or more of the embodiments described herein (e.g., fusion proteins), is provided in Table 9.

TABLE 9
The Amino Acid Sequence of Exemplary mIg heavy chain constant region
components and hIg light chain constant regions.

| | | SEQ |
|---|---|---|
| Description | Amino Acid Sequence | ID NO |
| mIgG1 CH1 Region | AKTTPPSVYPLAPGSAAQTNSMVTLGCLVKGYFPEPVTVTWNSG | 665 |
| | SLSSGVHTFPAVLQSDLYTLSSSVTVPSSPRPSETVTCNVAHPA | |
| | SSTKVDKKI | |
| mIgG1 Hinge Region | VPRDCGCKPCICT | 666 |
| mIgG1 CH2 Region | VPEVSSVFIFPPKPKDVLTITLTPKVTCVVVAISKDDPEVQFSW | 667 |
| | FVDDVEVHTAQTQPREEQFNSTFRSVSELPIMHQDWLNGKEFKC | |
| | RVNSAAFPAPIEKTISKTK | |
| mIgG1 CH3 Region | GRPKAPQVYTIPPPKEQMAKDKVSLTCMITDFFPEDITVEWQWN | 668 |
| With C-terminal Lysine | GQPAENYKNTQPIMNTNGSYFVYSKLNVQKSNWEAGNTFTCSVL | |
| | HEGLHNHHTEKSLSHSPGK | |
| mIgG1 CH3 Region | GRPKAPQVYTIPPPKEQMAKDKVSLTCMITDFFPEDITVEWQWN | 669 |
| Without C-terminal | GQPAENYKNTQPIMNTNGSYFVYSKLNVQKSNWEAGNTFTCSVL | |
| Lysine | HEGLHNHHTEKSLSHSPG | |
| mIgG1 CH2 Region + | VPEVSSVFIFPPKPKDVLTITLTPKVTCVVVAISKDDPEVQFSW | 670 |
| CH3 Region | FVDDVEVHTAQTQPREEQFNSTFRSVSELPIMHQDWLNGKEFKC | |
| With C-terminal Lysine | RVNSAAFPAPIEKTISKTKGRPKAPQVYTIPPPKEQMAKDKVSL | |
| | TCMITDFFPEDITVEWQWNGQPAENYKNTQPIMNTNGSYFVYSK | |
| | LNVQKSNWEAGNTFTCSVLHEGLHNHHTEKSLSHSPGK | |
| mIgG1 CH2 Region + | VPEVSSVFIFPPKPKDVLTITLTPKVTCVVVAISKDDPEVQFSW | 671 |
| CH3 Region | FVDDVEVHTAQTQPREEQFNSTFRSVSELPIMHQDWLNGKEFKC | |
| Without C-terminal | RVNSAAFPAPIEKTISKTKGRPKAPQVYTIPPPKEQMAKDKVSL | |
| Lysine | TCMITDFFPEDITVEWQWNGQPAENYKNTQPIMNTNGSYFVYSK | |
| | LNVQKSNWEAGNTFTCSVLHEGLHNHHTEKSLSHSPG | |
| mIgG1 Hinge Region + | VPRDCGCKPCICTVPEVSSVFIFPPKPKDVLTITLTPKVTCVVV | 672 |
| CH2 Region + CH3 | AISKDDPEVQFSWFVDDVEVHTAQTQPREEQFNSTFRSVSELPI | |
| Region | MHQDWLNGKEFKCRVNSAAFPAPIEKTISKTKGRPKAPQVYTIP | |
| With C-terminal Lysine | PPKEQMAKDKVSLTCMITDFFPEDITVEWQWNGQPAENYKNTQP | |
| | IMNTNGSYFVYSKLNVQKSNWEAGNTFTCSVLHEGLHNHHTEKS | |
| | LSHSPGK | |
| mIgG1 Hinge Region + | VPRDCGCKPCICTVPEVSSVFIFPPKPKDVLTITLTPKVTCVVV | 673 |
| CH2 Region + CH3 | AISKDDPEVQFSWFVDDVEVHTAQTQPREEQFNSTFRSVSELPI | |
| Region | MHQDWLNGKEFKCRVNSAAFPAPIEKTISKTKGRPKAPQVYTIP | |
| Without C-terminal | PPKEQMAKDKVSLTCMITDFFPEDITVEWQWNGQPAENYKNTQP | |
| Lysine | IMNTNGSYFVYSKLNVQKSNWEAGNTFTCSVLHEGLHNHHTEKS | |
| | LSHSPGK | |
| mIgG2a Hinge Region | EPRGPTIKPCPPCKCP | 674 |
| mIgG2a CH2 Region | APNAAGGPSVFIFLLKIKDVLMISLSPIVTCVVVDVSEDDPDVQ | 675 |
| | ISWFVNNVEVHTAQTQTHREDYNSTLRVVSALPIQHQDWMSGKE | |
| | FKCKVNNKDLPAPIERTISKPKGSVRAPQVYVLPPPEEEMTKKQ | |
| | VT | |
| mIgG2a CH3 Region | LTCMVTDFMPEDIYVEWTNNGKTELNYKNTEPVLDSDGSYFMYS | 676 |
| With C-terminal Lysine | KLRVEKKNWVERNSYSCSVVHEGLHNHHTTKSFSRTPGK | |
| mIgG2a CH3 Region | LTCMVTDFMPEDIYVEWTNNGKTELNYKNTEPVLDSDGSYFMYS | 678 |
| Without C-terminal | KLRVEKKNWVERNSYSCSVVHEGLHNHHTTKSFSRTPG | |
| Lysine | | |
| mIgG2a CH2 Region + | APNAAGGPSVFIFLLKIKDVLMISLSPIVTCVVVDVSEDDPDVQ | 679 |
| CH3 Region | ISWFVNNVEVHTAQTQTHREDYNSTLRVVSALPIQHQDWMSGKE | |
| With C-terminal Lysine | FKCKVNNKDLPAPIERTISKPKGSVRAPQVYVLPPPEEEMTKKQ | |
| | VTLTCMVTDFMPEDIYVEWTNNGKTELNYKNTEPVLDSDGSYFM | |
| | YSKLRVEKKNWVERNSYSCSVVHEGLHNHHTTKSFSRTPGK | |
| mIgG2a CH2 Region + | APNAAGGPSVFIFLLKIKDVLMISLSPIVTCVVVDVSEDDPDVQ | 680 |
| CH3 Region | ISWFVNNVEVHTAQTQTHREDYNSTLRVVSALPIQHQDWMSGKE | |
| Without C-terminal | FKCKVNNKDLPAPIERTISKPKGSVRAPQVYVLPPPEEEMTKKQ | |
| Lysine | VTLTCMVTDFMPEDIYVEWTNNGKTELNYKNTEPVLDSDGSYFM | |
| | YSKLRVEKKNWVERNSYSCSVVHEGLHNHHTTKSFSRTPG | |
| mIgG2a Hinge Region + | EPRGPTIKPCPPCKCPAPNAAGGPSVFIFLLKIKDVLMISLSPI | 681 |
| CH2 Region + CH3 | VTCVVVDVSEDDPDVQISWFVNNVEVHTAQTQTHREDYNSTLRV | |
| Region | VSALPIQHQDWMSGKEFKCKVNNKDLPAPIERTISKPKGSVRAP | |
| With C-terminal Lysine | QVYVLPPPEEEMTKKQVTLTCMVTDFMPEDIYVEWTNNGKTELN | |
| | YKNTEPVLDSDGSYFMYSKLRVEKKNWVERNSYSCSVVHEGLHN | |
| | HHTTKSFSRTPGK | |
| mIgG2a Hinge Region + | EPRGPTIKPCPPCKCPAPNAAGGPSVFIFLLKIKDVLMISLSPI | 682 |
| CH2 Region + CH3 | VTCVVVDVSEDDPDVQISWFVNNVEVHTAQTQTHREDYNSTLRV | |
| Region | VSALPIQHQDWMSGKEFKCKVNNKDLPAPIERTISKPKGSVRAP | |
| Without C-terminal | QVYVLPPPEEEMTKKQVTLTCMVTDFMPEDIYVEWTNNGKTELN | |
| Lysine | YKNTEPVLDSDGSYFMYSKLRVEKKNWVERNSYSCSVVHEGLHN | |
| | HHTTKSFSRTPG | |
| mIgG2a CH1 Region + | AKTTPPSVYPLAPGSAAQTNSMVTLGCLVKGYFPEPVTVTWNSG | 683 |
| Hinge Region + CH2 | SLSSGVHTFPAVLQSDLYTLSSSVTVPSSPRPSETVTCNVAHPA | |
| Region + CH3 Region | SSTKVDKKIEPRGPTIKPCPPCKCPAPNAAGGPSVFIFLLKIKD | |
| With C-terminal Lysine | VLMISLSPIVTCVVVDVSEDDPDVQISWFVNNVEVHTAQTQTHR | |
| | EDYNSTLRVVSALPIQHQDWMSGKEFKCKVNNKDLPAPIERTIS | |
| | KPKGSVRAPQVYVLPPPEEEMTKKQVTLTCMVTDFMPEDIYVEW | |
| | TNNGKTELNYKNTEPVLDSDGSYFMYSKLRVEKKNWVERNSYSC | |
| | SVVHEGLHNHHTTKSFSRTPGK | |
| mIgG2a CH1 Region + | AKTTPPSVYPLAPGSAAQTNSMVTLGCLVKGYFPEPVTVTWNSG | 684 |
| Hinge Region + CH2 | SLSSGVHTFPAVLQSDLYTLSSSVTVPSSPRPSETVTCNVAHPA | |
| Region + CH3 Region | SSTKVDKKIEPRGPTIKPCPPCKCPAPNAAGGPSVFIFLLKIKD | |
| Without C-terminal | VLMISLSPIVTCVVVDVSEDDPDVQISWFVNNVEVHTAQTQTHR | |
| Lysine | EDYNSTLRVVSALPIQHQDWMSGKEFKCKVNNKDLPAPIERTIS | |
| | KPKGSVRAPQVYVLPPPEEEMTKKQVTLTCMVTDFMPEDIYVEW | |
| | TNNGKTELNYKNTEPVLDSDGSYFMYSKLRVEKKNWVERNSYSC | |
| | SVVHEGLHNHHTTKSFSRTPG | |
| Ig light chain kappa | RADAAPTVSIFPPSSEQLTSGGASVVCFLNNFYPKDINVKWKID | 685 |
| constant region (κCL) | GSERQNGVLNSWTDQDSKDSTYSMSSTLTLTKDEYERHNSYTCE | |
| | ATHKTSTSPIVKSFNRNEC | |
| Ig light chain kappa | QPKSSPSVTLFPPSSEELETNKATLVCTITDFYPGVVTVDWKVD | 686 |
| constant region (λCL) | GTPVTQGMETTQPSKQSNNKYMASSYLTLTARAWERHSSYSCQV | |
| | THEGHTVEKSLSRADCS | |

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 9. In some embodiments, a fusion protein described herein comprises a protein (or a functional fragment, functional variant, or functional fragment/variant thereof) comprising an amino acid sequence at least 85% identical to the amino acid sequence of a protein set forth in Table 9. In some embodiments, a fusion protein described herein comprises a protein ((or a functional fragment, functional variant, or functional fragment/variant thereof) comprising an amino acid sequence at least 90% identical to the amino acid sequence of a protein set forth in Table 9. In some embodiments, a fusion protein described herein comprises a protein (or a functional fragment, functional variant, or functional fragment/variant thereof) comprising an amino acid sequence at least 95% identical to the amino acid sequence of a protein set forth in Table 9. In some embodiments, a fusion protein described herein comprises a protein (or a functional fragment, functional variant, or functional fragment/variant thereof) comprising an amino acid sequence at least 99% identical to the amino acid sequence of a protein set forth in Table 9.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 9. In some embodiments, a fusion protein described herein comprises a protein (or a functional fragment, functional variant, or functional fragment/variant thereof) comprising an amino acid sequence 85% identical to the amino acid sequence of a protein set forth in Table 9. In some embodiments, a fusion protein described herein comprises a protein ((or a functional fragment, functional variant, or functional fragment/variant thereof) comprising an amino acid sequence 90% identical to the amino acid sequence of a protein set forth in Table 9. In some embodiments, a fusion protein described herein comprises a protein (or a functional fragment, functional variant, or functional fragment/variant thereof) comprising an amino acid sequence 95% identical to the amino acid sequence of a protein set forth in Table 9. In some embodiments, a fusion protein described herein comprises a protein (or a functional fragment, functional variant, or functional fragment/variant thereof) comprising an amino acid sequence 99% identical to the amino acid sequence of a protein set forth in Table 9. In some embodiments, a fusion protein described herein comprises a protein (or a functional fragment, functional variant, or functional fragment/variant thereof) comprising an amino acid sequence 100% identical to the amino acid sequence of a protein set forth in Table 9.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence of a protein set forth in Table 9. In some embodiments, a fusion protein described herein comprises a protein (or a functional fragment, functional variant, or functional fragment/variant thereof) consisting of an amino acid sequence at least 85% identical to the amino acid sequence of a protein set forth in Table 9. In some embodiments, a fusion protein described herein comprises a protein (or a functional fragment, functional variant, or functional fragment/variant thereof) consisting of an amino acid sequence at least 90% identical to the amino acid sequence of a protein set forth in Table 9. In some embodiments, a fusion protein described herein comprises a protein (or a functional fragment, functional variant, or functional fragment/variant thereof) consisting of an amino acid sequence at least 95% identical to the amino acid sequence of a protein set forth in Table 9. In some embodiments, a fusion protein described herein comprises a protein (or a functional fragment, functional variant, or functional fragment/variant thereof) consisting of an amino acid sequence at least 99% identical to the amino acid sequence of a protein set forth in Table 9.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence of a protein set forth in Table 9. In some embodiments, a fusion protein described herein comprises a protein (or a functional fragment, functional variant, or functional fragment/variant thereof) consisting of an amino acid sequence 85% identical to the amino acid sequence of a protein set forth in Table 9. In some embodiments, a fusion protein described herein comprises a protein (or a functional fragment, functional variant, or functional fragment/variant thereof) consisting of an amino acid sequence 90% identical to the amino acid sequence of a protein set forth in Table 9. In some embodiments, a fusion protein described herein comprises a protein (or a functional fragment, functional variant, or functional fragment/variant thereof) consisting of an amino acid sequence 95% identical to the amino acid sequence of a protein set forth in Table 9. In some embodiments, a fusion protein described herein comprises a protein (or a functional fragment, functional variant, or functional fragment/variant thereof) consisting of an amino acid sequence 99% identical to the amino acid sequence of a protein set forth in Table 9. In some embodiments, a fusion protein described herein comprises a protein (or a functional fragment, functional variant, or functional fragment/variant thereof) consisting of an amino acid sequence 100% identical to the amino acid sequence of a protein set forth in Table 9.

In some embodiments, a fusion protein described herein comprises a protein (or a functional fragment, functional variant, or functional fragment/variant thereof) comprising the amino acid sequence of a protein set forth in Table 9, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 9, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 9, and further consisting of at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 9, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 9, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 9, and further comprising or no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence of a protein set forth in Table 9, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 9, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 9, and further consisting of at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 9, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 9, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 9, and further comprising or no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence at least 85% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence at least 90% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence at least 95% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence at least 99% identical to the amino acid sequence set forth in any one of SEQ ID NOS: 665-676 or 678-686.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence 85% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence 90% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence 95% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence 99% identical to the amino acid sequence set forth in any one of SEQ ID NOS: 665-676 or 678-686. In some embodiments, a fusion protein described herein comprises a protein (or a functional fragment, functional variant, or functional fragment/variant thereof) comprising an amino acid sequence 100% identical to the amino acid sequence set forth in any one of SEQ ID NOS: 665-676 or 678-686.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence at least 85% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence at least 90% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence at least 95% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence at least 99% identical to the amino acid sequence set forth in any one of SEQ ID NOS: 665-676 or 678-686.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence 85% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence 90% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence 95% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence 99% identical to the amino acid sequence set forth in any one of SEQ ID NOS: 665-676 or 678-686. In some embodiments, a fusion protein described herein comprises a protein (or a functional fragment, functional variant, or functional fragment/variant thereof) consisting of an amino acid sequence 100% identical to the amino acid sequence set forth in any one of SEQ ID NOS: 665-676 or 678-686.

In some embodiments, a fusion protein described herein comprises a protein (or a functional fragment, functional variant, or functional fragment/variant thereof) comprising the amino acid sequence set forth in any one of SEQ ID NOS: 665-676 or 678-686, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 665-676 or 678-686, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 665-676 or 678-686, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 665-676 or 678-686, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 665-676 or 678-686, and further comprising or consists of no more than 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence set forth in any one of SEQ ID NOS: 665-676 or 678-686, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 665-676 or 678-686, and further comprising at least 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 665-676 or 678-686, and further comprising 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 665-676 or 678-686, and further consisting of 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 665-676 or 678-686, and further consisting of no more than 1, 2, 3, 4, 5, 6, 7, 8, 9, or 10 amino acid variations (e.g., substitutions, additions, deletions, etc.).

In some embodiments, a fusion protein described herein comprises a CH3 region (e.g., comprises an Fc region; a hinge region, CH2 region, and CH3 region, etc.), wherein the CH3 region lacks the C-terminal lysine (e.g., residue 232 of SEQ ID NO: 672, EU numbering according to Kabat; or e.g., residue 229 of SEQ ID NO: 681, EU numbering according to Kabat). In some embodiments, the CH3 region further lacks the C-terminal glycine (e.g., residue 231 of SEQ ID NO: 672, EU numbering according to Kabat; or e.g., residue 228 of SEQ ID NO: 681, EU numbering according to Kabat).

### 5.4.3 Half-Life Extension

In some embodiments, the Ig (e.g., hIg, mIg) Fc region of a fusion protein described herein (an Fc region, an antibody, etc.) exhibits enhanced serum half-life, e.g., relative to a reference Ig (e.g., hIg, mIg) Fc region (e.g., a wild-type Ig (e.g., hIg, mIg) Fc region).

Standard in vitro and/or in vivo assays known in the art can be conducted to evaluate serum half-life. See, e.g., Ko S, Jo M, Jung S T. Recent Achievements and Challenges in Prolonging the Serum Half-Lives of Therapeutic IgG Antibodies Through Fc Engineering. BioDrugs. 2021; 35(2):147-157. doi:10.1007/s40259-021-00471-0, the entire contents of which are incorporated herein by reference for all purposes.

In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) (e.g., relative to a reference Ig (e.g., hIg, mIg) Fc region (e.g., a wild-type Ig (e.g., hIg, mIg) Fc region)).

In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of from about 5.5-6.5 (e.g., relative to a reference Ig (e.g., hIg, mIg) Fc region (e.g., a wild-type Ig (e.g., hIg, mIg) Fc region)). In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of from about 5.5-6.5 and no substantial change in binding affinity for the FcRn receptor (e.g., the human FcRn receptor) at a pH of from about 7.0-7.5 (e.g., relative to a reference Ig (e.g., hIg, mIg) Fc region (e.g., a wild-type Ig (e.g., hIg, mIg) Fc region)). In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of from about 6.0-6.5 (e.g., relative to a reference Ig (e.g., hIg, mIg) Fc region (e.g., a wild-type Ig (e.g., hIg, mIg) Fc region)) and a decrease in binding affinity for the FcRn receptor (e.g., the human FcRn receptor) at a pH of from about 7.0-7.5 (e.g., relative to a reference Ig (e.g., hIg, mIg) Fc region (e.g., a wild-type Ig (e.g., hIg, mIg) Fc region)).

In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of about 6 (e.g., relative to a reference Ig (e.g., hIg, mIg) Fc region (e.g., a wild-type Ig (e.g., hIg, mIg) Fc region)). In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of about 6 and no substantial change in binding affinity for the FcRn receptor (e.g., the human FcRn receptor) at a pH of about 7.4 (e.g., relative to a reference Ig (e.g., hIg, mIg) Fc region (e.g., a wild-type Ig (e.g., hIg, mIg) Fc region)). In some embodiments, the Ig (e.g., hIg, mIg) Fc region of a fusion protein described herein (an Fc region, an antibody, etc.) exhibits enhanced serum half-life (e.g., relative to a reference Ig (e.g., hIg, mIg) Fc region (e.g., a wild-type Ig (e.g., hIg, mIg) Fc region)) through enhanced binding affinity for the FcRn receptor (e.g., the human FcRn receptor) at a pH of about 6 (e.g., relative to a reference Ig (e.g., hIg, mIg) Fc region (e.g., a wild-type Ig (e.g., hIg, mIg) Fc region)) and a decrease in binding affinity for the FcRn receptor (e.g., the human FcRn receptor) at a pH of about 7.4 (e.g., relative to a reference Ig (e.g., hIg, mIg) Fc region (e.g., a wild-type Ig (e.g., hIg, mIg) Fc region)).

In some embodiments, the Ig (e.g., hIg, mIg) Fc region of a fusion protein described herein comprises one or more amino acid variation (e.g., relative to a reference Ig (e.g., hIg, mIg) Fc region (e.g., a wild-type Ig (e.g., hIg, mIg) Fc region)) that enhances serum half-life of the fusion protein (e.g., relative to a reference Ig (e.g., hIg, mIg) Fc region (e.g., a wild-type Ig (e.g., hIg, mIg) Fc region)).

In some embodiments, the Ig (e.g., hIg, mIg) Fc region of a fusion protein described herein comprises one or more amino acid variation (e.g., relative to a reference Ig (e.g., hIg, mIg) Fc region (e.g., a wild-type Ig (e.g., hIg, mIg) Fc region)) that enhances serum half-life of the fusion protein (e.g., relative to a reference Ig (e.g., hIg, mIg) Fc region (e.g., a wild-type Ig (e.g., hIg, mIg) Fc region)) through altered binding to the FcRn receptor (e.g., as described herein) (e.g., an FcRn binding profile described herein).

Exemplary amino acid variations of an Ig (e.g., hIg, mIg) Fc region that enhance serum half-life of the Ig Fc region (or a protein comprising the same) are known in the art. See, e.g., Ko 2021 (and references cited therein) (including e.g., Table 1 of Ko 2021); Xinhua Wang, Mary Mathieu, Randall J Brezski, IgG Fc engineering to modulate antibody effector functions, Protein & Cell, Volume 9, Issue 1, January 2018, Pages 63-73, https://doi.org/10.1007/s13238-017-0473-8; U.S. Pat. No. 8,546,543B2; WO2024059652A1; U.S. Ser. No. 11/591,368 (e.g., H433K/N434F); Ko, S., Park, S., Sohn, M. H. et al. An Fc variant with two mutations confers prolonged serum half-life and enhanced effector functions on IgG antibodies. Exp Mol Med 54, 1850-1861 (2022). https://doi.org/10.1038/s12276-022-00870-5; the entire contents of each of which is incorporated herein by reference for all purposes.

Table 10 below, provides exemplary amino acid substitutions (and combinations thereof) and glycoengineering that can be utilized to extend half-life of proteins (e.g., fusion proteins described herein) comprising an Ig Fc region (or fragment thereof). Amino acids in Table 10 are numbered according to the EU numbering scheme. The amino acid substitutions set forth in Table 10 are with reference to an IgG1 Fc region (except where noted). However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 10
Exemplary hIg Fc Variations to Extend Half-Life.

| | |
|---|---|
| | Exemplary Effects on Effector |
| Variation/Glycoengineering | Function (Non-Limiting) |

Amino Acid Variations

| | |
|---|---|
| R435H | Extended Half-Life |
| N434A | Extended Half-Life |
| N434W | Extended Half-Life |
| M252Y/S254T/T256E | Extended Half-Life |
| M252Y/T256D | Extended Half-Life |
| M428L/N434S | Extended Half-Life |
| E294Δ/R307P/N434Y | Extended Half-Life |
| T256D/T307Q | Extended Half-Life |
| T256D/T307W | Extended Half-Life |
| T256N/A378V/S383N/N434Y | Extended Half-Life |
| T307Q/Q311V/A378V | Extended Half-Life |
| T256D/H286D/T307R/Q311V/A378V | Extended Half-Life |
| L309D/Q311H/N434S | Extended Half-Life |
| H433K/N434F | Extended Half-Life |
| H433K/N434F (IgG4) | Extended Half-Life |
| E294Δ | Extended Half-Life |

In some embodiments, the Ig Fc region is a hIg Fc region. In some embodiments, the hIg Fe (e.g., IgG1 Fc) region comprises any one or more of the amino acid substitutions set forth in Table 10 (i.e., any one or more amino acid substitution set forth in any set of amino acid substitutions set forth in Table 10). In some embodiments, the hIg Fe (e.g., IgG1 Fc) comprises any one or more of the sets of amino acid substitutions set forth in Table 10. In some embodiments, the hIg Fe (e.g., IgG1 Fc) region comprises any one or more of the glycosylation changes set forth in Table 10.

For example, amino acid variations include, but are not limited to, M428L/N434S, EU numbering according to Kabat; M252Y/S254T/T256E, EU numbering according to Kabat; N434A, EU numbering according to Kabat; N434W, EU numbering according to Kabat; T256D/T307Q, EU numbering according to Kabat; T256D/T307W, EU numbering according to Kabat; M252Y/T256D, EU numbering according to Kabat; T307Q/Q311V/A378V, EU numbering according to Kabat; T256D/H286D/T307R/Q311V/A378V, EU numbering according to Kabat; and L309D/Q311H/N434S, EU numbering according to Kabat. Further amino acid modifications include, H433K/N434F (of IgG1) or H433K/N434F (of IgG4), EU numbering according to Kabat.

In some embodiments, the Ig (e.g., hIg, mIg) Fc region of a fusion protein described herein comprises one or more alteration (including various post-translational modifications e.g., glycosylation, sialylation) that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein comprises one or more post-translational modification (e.g., glycosylation, sialylation) that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein comprises altered glycosylation that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein comprises altered lipidation that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein comprises altered sialylation that mediates enhanced serum half-life, e.g., relative to a reference (e.g., wild type) Ig (e.g., hIg, mIg) Fc region. In some embodiments, the Ig (e.g., hIg, mIg) Fc region of a fusion protein described herein is pegylated, which mediates enhanced serum half-life, e.g., relative to a reference (e.g., wild type) Ig (e.g., hIg, mIg) Fc region.

### 5.4.4 Ig Fc Effector Function

In some embodiments, the Ig (e.g., hIg, mIg) Fc region of a fusion protein described herein exhibits modulation (e.g., a decrease or increase) of one or more Fc effector function, e.g., relative to a reference (e.g., wild type) Ig (e.g., hIg, mIg) Fc region. Exemplary Ig (e.g., hIg, mIg) Fc effector functions include, but are not limited to, antibody dependent cellular cytotoxicity (ADCC), antibody dependent cellular phagocytosis (ADCP), complement dependent cytotoxicity (CDC), and binding affinity to one or more human Fc receptor (e.g., an Fc receptor (e.g., FcγRI, FcγRIIa, FcγRIIc, FcγRIIIa, and/or FcγRIIIb (e.g., FcγRI, FcγIIa, and/or FcγIIIa))).

Standard in vitro and/or in vivo assays known in the art can be conducted to evaluate Ig Fc effector function, including, any one or more of ADCC, CDC, ADCP, Fc receptor (e.g., Fc receptor) binding/binding affinity, and C1q binding affinity.

For example, ADCC activity can be assessed utilizing standard (radioactive and non-radioactive) methods known in the art (see, e.g., WO2006/082515, WO2012/130831), the entire contents of each of which is incorporated by reference herein for all purposes). For example, ADCC activity can be assessed using a chromium-5 (⁵¹Cr) assay. Briefly, ⁵¹Cr is pre-loaded into target cells expressing CD20, NK cells are added to the culture, and radioactivity in the cell culture supernatant is assessed (indicative of lysis of the target cells by the NK cells). Similar non-radioactive assays can also be utilized that employ a similar method, but the target cells are pre-loaded with fluorescent dyes, such as calcein-AM, CFSE, BCECF, or lanthanide flurophore (Europium). See, e.g., Parekh, Bhavin S et al. “Development and validation of an antibody-dependent cell-mediated cytotoxicity-reporter gene assay.” mAbs vol. 4,3 (2012): 310-8. Doi:10.4161/mabs.19873, the entire contents of which is incorporated by reference herein for all purposes. Exemplary commercially available non-radioactive assays include, for example, ACTI™ non-radioactive cytotoxicity assay for flow cytometry (Cell Technology, Inc. Mountain View, Calif.; and CytoTox 96® non-radioactive cytotoxicity assay (Promega, Madison, Wis.). Additional non-limiting examples of in vitro assays that can be used to assess ADCC activity of a fusion protein described herein include those described in U.S. Pat. Nos. 5,500,362; 5,821,337; Hellstrom, I., et al., Proc. Nat'l Acad. Sci. USA 83 (1986) 7059-7063; Hellstrom, I., et al., Proc. Nat'l Acad. Sci. USA 82 (1985) 1499-1502; and Bruggemann, M., et al., J. Exp. Med. 166 (1987) 1351-1361, the entire contents of each of which is incorporated by reference herein. Alternatively, or additionally, ADCC activity of a fusion protein described herein may be assessed in vivo, e.g., in an animal model such as that disclosed in Clynes, et al., Proc. Nat'l Acad. Sci. USA 95 (1998) 652-656, the entire contents of which is incorporated by reference herein for all purposes.

C1q binding assays can be utilized to assess the ability of a hIg fusion protein described herein to bind C1q (or bind with less affinity than a reference fusion protein) and hence lack (or have decreased) CDC activity. The binding of a hIg fusion protein described herein to C1q can be determined by a variety of in vitro assays (e.g., biochemical or immunological based assays) known in the art for determining Fc-C1q interactions, including e.g., equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4^{th} Ed., Lippincott-Raven, Philadelphia (1999), the entire contents of which is incorporated by reference herein. For example, see, e.g., C1q and C3c binding ELISAs described in WO2006/029879 and WO2005/100402, the entire contents of each of which is incorporated by reference herein for all purposes. Additional CDC activity assays include those described in e.g., Gazzano-Santoro, et al., J. Immunol. Methods 202 (1996) 163; Cragg, M. S., et al., Blood 101 (2003) 1045-1052; and Cragg, M. S., and Glennie, M. J., Blood 103 (2004) 2738-2743), the entire contents of each of which is incorporated by reference herein for all purposes.

ADCP activity can be measured by in vitro or in vivo methods known in the art and also commercially available assays (see, e.g., van de Donk N W, Moreau P, Plesner T, et al. “Clinical efficacy and management of monoclonal antibodies targeting CD38 and SLAMF7 in multiple myeloma,” Blood, 127(6):681-695 (2016), the entire contents of each of which is incorporated by reference herein for all purposes). For example, a primary cell based ADCP assay can be used in which fresh human peripheral blood mononuclear cells (PBMCs) are isolated, monocytes isolated and differentiated in culture to macrophages using standard procedures. The macrophages are fluorescently labeled added to cultures containing fluorescently labeled target cells expressing CD20 and a fusion protein described herein. Phagocytosis events can be analyzed using FACS screening and/or microscopy. A modified reporter version of the above described assay can also be used that employs an engineered cell line that stably expresses FcγRIIa (CD32a) as the effector cell line (e.g., an engineered T cell line, e.g., THP-1), removing the requirement for primary cells. Exemplary ADCP assays are described in e.g., Ackerman, M. E. et al. A robust, high-throughput assay to determine the phagocytic activity of clinical antibody samples. J. Immunol. Methods 366, 8-19 (2011); and Mcandrew, E. G. et al. Determining the phagocytic activity of clinical antibody samples. J. Vis. Exp. 3588 (2011). Doi:10.3791/3588; the entire contents of each of which is incorporated by reference herein.

Binding of an Ig Fc fusion protein described herein to an Ig Fc receptor can be determined by a variety of in vitro assays (e.g., biochemical or immunological based assays) known in the art for determining Fc-Fc receptor interactions, i.e., specific binding of an Ig Fc region to an Ig Fc receptor. Common assays include equilibrium methods (e.g., enzyme-linked immunosorbent assay (ELISA) or radioimmunoassay (RIA)), or kinetic methods (e.g., surface plasmon resonance (SPR) analysis), and other methods such as indirect binding assays, competitive inhibition assays, fluorescence resonance energy transfer (FRET), gel electrophoresis, and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in e.g., Paul, W. E., ed., Fundamental Immunology, 4″ Ed., Lippincott-Raven, Philadelphia (1999), the entire contents of which is incorporated by reference herein for all purposes.

### 5.4.4.1 Reduced Ig Fc Effector Function

In some embodiments, the Ig Fc region exhibits a reduction in (e.g., a decrease in) activity of one or more Fc effector functions or no detectable activity of one or more Fc effector functions. As described above, exemplary Ig Fc effector functions include, but are not limited to, ADCC, ADCP, CDC, binding affinity to C1q, and binding affinity to one or more human Fc receptor (e.g., an Fc receptor (e.g., FcγRI, FcγRIIa, FcγRIIb, FcγRIIc, FcγRIIIa, and/or FcγRIIIb)).

In some embodiments, the Ig Fc region is modified (e.g., comprises one or more variation (e.g., one or more amino acid substitution, deletion, addition, etc.); altered glycosylation)) (referred to herein as a “modified Ig Fc region”). In some embodiments, the modification (e.g., the variation (e.g., one or more amino acid substitution, deletion, addition, etc.); altered glycosylation) decreases or abolishes one or more Fc effector function (e.g., relative to a reference hIg Fc that does not comprise the modification (e.g., the one or more variation (e.g., the one or more amino acid substitution, deletion, addition, etc.; the altered glycosylation))). In some embodiments, the modification (e.g., the variation (e.g., one or more amino acid substitution, deletion, addition, etc.); altered glycosylation) decreases one or more Fc effector function, relative to a reference hIg Fc that does not comprise the modification (e.g., the one or more variation (e.g., the one or more amino acid substitution, deletion, addition, etc.; the altered glycosylation)). In some embodiments, the modification (e.g., the variation (e.g., one or more amino acid substitution, deletion, addition, etc.); altered glycosylation) abolishes one or more Fc effector function.

In some embodiments, a fusion protein (e.g., described herein) comprising a modified Ig (e.g., hIg) Fc region exhibits no detectable or decreased ADCC compared to a reference multispecific protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable ADCC. In some embodiments, a fusion protein (e.g., described herein) comprising a modified Ig (e.g., hIg) Fc exhibits decreased ADCC compared to a reference multispecific protein that does not comprise the Ig (e.g., hIg) Fc modification (e.g., the one or more variation (e.g., one or more amino acid substitution, deletion, or addition)).

In some embodiments, a fusion protein (e.g., described herein) comprising a modified Ig (e.g., hIg) Fc exhibits no detectable or decreased CDC compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable CDC. In some embodiments, a fusion protein (e.g., described herein) comprising a modified Ig (e.g., hIg) Fc exhibits decreased CDC compared to a reference fusion protein that does not comprise the Ig (e.g., hIg) Fc modification (e.g., the one or more variation (e.g., one or more amino acid substitution, deletion, or addition)).

In some embodiments, a fusion protein (e.g., described herein) comprising a modified Ig (e.g., hIg) Fe exhibits no detectable or decreased ADCP compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable ADCP. In some embodiments, a fusion protein (e.g., described herein) comprising a modified Ig (e.g., hIg) Fc exhibits decreased ADCP compared to a reference fusion protein that does not comprise the Ig (e.g., hIg) Fc modification (e.g., the one or more variation (e.g., one or more amino acid substitution, deletion, or addition)).

In some embodiments, a fusion protein (e.g., described herein) comprising a modified Ig (e.g., hIg) Fc exhibits decreased or no detectable binding affinity to one or more Fc receptor (e.g., human Fc receptor) (e.g., an Fcγ receptor (e.g., FcγRI, FcγRIIa, FcγRIIb, FcγRIIc, FcγRIIIa, and/or FcγRIIIb)) compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable binding to one or more Fc receptor (e.g., human Fc receptor) (e.g., an Fcγ receptor (e.g., FcγRI, FcγRIIa, FcγRIIb, FcγRIIc, FcγRIIIa, and/or FcγRIIIb)). In some embodiments, a fusion protein (e.g., described herein) comprising a modified Ig (e.g., hIg) Fc exhibits decreased binding affinity to one or more Fc receptor (e.g., human Fc receptor) (e.g., an Fcγ receptor (e.g., FcγRI, FcγRIIa, FcγRIIb, FcγRIIc, FcγRIIIa, and/or FcγRIIIb)) compared to a reference fusion protein that does not comprise the Ig (e.g., hIg) Fc modification (e.g., the one or more variation (e.g., one or more amino acid substitution, deletion, or addition)).

In some embodiments, a fusion protein (e.g., described herein) comprising a modified Ig (e.g., hIg) Fc exhibits enhanced binding affinity to one or more Fc receptor (e.g., human Fc receptor) (e.g., an Fcγ receptor (e.g., FcγRIIb)) compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising the modified Ig (e.g., hIg) Fc exhibits reduced binding affinity to one or more Fc receptor (e.g., human Fc receptor) (e.g., an Fcγ receptor (e.g., FcγRI, FcγRIIa, FcγRIIIa and/or FcγRIIIb) compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising the modified Ig (e.g., hIg) Fc exhibits no detectable binding to one or more Fc receptor (e.g., human Fc receptor) (e.g., an Fcγ receptor (e.g., FcγRI, FcγRIIa, FcγRIIIa and/or FcγRIIIb).

In some embodiments, a fusion protein (e.g., described herein) comprising a modified Ig (e.g., hIg) Fe exhibits enhanced binding affinity to FcγRIIb compared to a reference fusion protein that does not comprise the hIg Fc modification (e.g., the one or more variation (e.g., one or more amino acid substitution, deletion, or addition)).

In some embodiments, a fusion protein (e.g., described herein) comprising a modified Ig (e.g., hIg) Fc exhibits reduced binding affinity to FcγRI, FcγRIIa, FcγRIIIa, and/or FcγRIIIb compared to a reference fusion protein that does not comprise the hIg Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable binding to FcγRI, FcγRIIa, FcγRIIIa, and/or FcγRIIIb.

In some embodiments, a fusion protein (e.g., described herein) comprising a modified Ig (e.g., hIg) Fc exhibits reduced binding affinity to FcγRI compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable binding to FcγRI.

In some embodiments, a fusion protein (e.g., described herein) comprising a modified Ig (e.g., hIg) Fc exhibits reduced binding affinity to FcγRIIa compared to a reference multispecific protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable binding to FcγRIIa.

In some embodiments, a fusion protein (e.g., described herein) comprising a modified Ig (e.g., hIg) Fc exhibits reduced binding affinity to FcγRIIb compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable binding to FcγRIIb.

In some embodiments, a fusion protein (e.g., described herein) comprising a modified Ig (e.g., hIg) Fe exhibits reduced binding affinity to FcγRIIIa compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable binding to FcγRIIIa.

In some embodiments, a fusion protein (e.g., described herein) comprising a modified Ig (e.g., hIg) Fc exhibits reduced binding affinity to FcγRIIIb compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable binding to FcγRIIIb.

In some embodiments, a fusion protein (e.g., described herein) comprising a modified Ig (e.g., hIg) Fc exhibits decreased binding affinity to C1q compared to a reference fusion protein that does not comprise the Ig (e.g., hIg) Fc modification (e.g., the one or more variation (e.g., one or more amino acid substitution, deletion, or addition)). In some embodiments, a fusion protein (e.g., described herein) comprising a modified Ig (e.g., hIg) Fc exhibits no detectable binding to C1q.

Amino acid substitutions that decrease or abolish one or more Ig (e.g., hIg) Fc region effector function are known in the art. See for example, Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, Liu R, Oldham R J, Teal E, Beers S A, Cragg M S. Fc-Engineering for Modulated Effector Functions-Improving Antibodies for Cancer Treatment. Antibodies (Basel). 2020; 9(4):64. Published 2020 Nov. 17. doi:10.3390/antib9040064; the full contents of each of which is incorporated by reference herein for all purposes, see more particularly for example, e.g., Table 3 of Saunders.

Table 11 below, provides exemplary amino acid substitutions (and combinations thereof) and glycoengineering that can be utilized to decrease one or more hIg Fc effector function. Amino acids in Table 11 are numbered according to the EU numbering scheme. The effects on effector function set forth in Table 11 are exemplary only and not intended to be limiting. The amino acid substitutions set forth in Table 11 are with reference to an IgG1 Fe region (except where noted). However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 11
Exemplary hIg Fc Variations and Glycoengineering
to Decreases Effector Function.

| | |
|---|---|
| Variation/ | Exemplary Effects on Effector |
| Glycoengineering | Function (Non-Limiting) |

Amino Acid Substitutions

| | |
|---|---|
| L235E | Decreased binding to cell surface FcγRs |
| | Decreased ADCC |
| L234A/L235A | Decreased binding to FcγRI, RII, III |
| | Decreased ADCC, ADCP, CDC |
| S228P/L235E (IgG4) | Decreased binding to FcγRI |
| L234A/L235A/P329G | Eliminates binding to Decreased |
| | binding to FcγRI, RII, III, C1q |
| | Decreased ADCP |
| L234A/L235A/P329A | Eliminates binding to Decreased |
| | binding to FcγRI, RII, III, C1q |
| | Decreased ADCP |
| L235A/G237A/P329G | Reduced ADCC, ADCP, CDC |
| L235A/G237A/P329A | Reduced ADCC, ADCP, CDC |
| P331S/L234E/L235F | Eliminates binding to Decreased |
| | binding to FcγRI, RII, III, C1q |
| | Decreased CDC |
| D235A | Decreased binding to FcγRI, RII, III |
| | Reduced ADCC, ADCP |
| G237A | Decreased binding to FcγRII |
| | Decreased ADCP |
| E318A | Decreased binding to FcγRII |
| | Decreased ADCP |
| E233P | Decreased binding to FcγRI, RII, III |
| G236R/L328R | Decreased binding to all FcγRs |
| | Decreased ADCC |
| A330L | Decreased C1q binding |
| | Decreased CDC |
| D270A | Decreased C1q binding |
| | Decreased CDC |
| K332A | Decreased C1q binding |
| | Decreased CDC |
| P329A | Decreased C1q binding |
| | Decreased CDC |
| P331A | Decreased C1q binding |
| | Decreased CDC |
| V264A | Decreased C1q binding |
| | Decreased CDC |
| F241A | Decreased C1q binding |
| | Decreased CDC |
| N297A | Decreased binding to FcγRI, RIIIa |
| | Decreased C1q binding |
| | Decreased ADCC |
| | Decreased ADCP |
| | Decreased CDC |
| N297G | Decreased binding to FcγRI, RIIIa |
| | Decreased C1q binding |
| | Decreased ADCC |
| | Decreased ADCP |
| | Decreased CDC |
| N297Q | Decreased binding to FcγRI, RIIIa |
| | Decreased C1q binding |
| | Decreased ADCC |
| | Decreased ADCP |
| | Decreased CDC |
| S228P/F234A/L235A | Decreased binding to FcγRI, RIIa, RIIIa |
| (IgG4) | Decreased ADCC |
| | Decreased CDC |
| S228P/F234A/L235E | Decreased binding to FcγRI, RIIa, RIIIa |
| (IgG4) | Decreased ADCC |
| | Decreased CDC |

Glycoengineering

| | |
|---|---|
| High mannose | Decreased C1q binding |
| glycosylation | Decreased CDC |

In some embodiments, the Ig Fc region is a hIg Fc region. In some embodiments, the hIg Fe (e.g., IgG1 Fc) region comprises any one or more of the amino acid substitutions set forth in Table 13 (i.e., any one or more amino acid substitution set forth in any set of amino acid substitutions set forth in Table 11). In some embodiments, the hIg Fe (e.g., IgG1 Fc) comprises any one or more of the sets of amino acid substitutions set forth in Table 11. In some embodiments, the hIg Fe (e.g., IgG1 Fc) region comprises any one or more of the glycosylation changes set forth in Table 11.

In some embodiments, the modified Ig Fc fusion protein comprises a hIg Fc region comprising one or more amino acid variation. In some embodiments, the modified hIg Fc fusion protein comprises a hIg4 Fc region comprising one or more amino acid variation. In some embodiments, the hIgG4 Fc region comprises an amino acid substitution at amino acid positions S228, F234, and/or L235, EU numbering according to Kabat. In some embodiments, the hIgG4 Fc region comprises the following amino acid substitutions S228P, F234A, and/or L235A, EU numbering according to Kabat. In some embodiments, the hIgG4 Fc region comprises the following amino acid substitutions S228P, F234A, and/or L235E, EU numbering according to Kabat. In some embodiments, the hIgG4 Fc comprises the following amino acid substitutions S228P and/or L235E, EU numbering according to Kabat.

In some embodiments, the S228P variation stabilized the hinge region. See, e.g., Silva, John-Paul et al. “The S228P mutation prevents in vivo and in vitro IgG4 Fab-arm exchange as demonstrated using a combination of novel quantitative immunoassays and physiological matrix preparation.” The Journal of biological chemistry vol. 290,9 (2015): 5462-9. doi:10.1074/jbc.M114.600973, the entire contents of which is incorporated herein by reference for all purposes.

In some embodiments, the modified hIg Fc fusion protein comprises a hIgG1 Fc region comprising one or more amino acid variations. In some embodiments, the hIgG1 Fc region comprises an amino acid substitution at amino acid positions L234, L235, and/or P329, EU numbering according to Kabat. In some embodiments, the hIgG1 Fc region comprises the following amino acid substitutions L234A and/or L235A, EU numbering according to Kabat. In some embodiments, the hIgG1 Fc region comprises the following amino acid substitutions L234A, L235A, and P329G, EU numbering according to Kabat. In some embodiments, the hIgG1 Fc region comprises the following amino acid substitutions L234A, L235A, and P329A, EU numbering according to Kabat.

In some embodiments, the modified hIg Fc fusion protein comprises a hIgG1 Fc region comprising one or more amino acid variations. In some embodiments, the hIgG1 Fc region comprises an amino acid substitution at amino acid positions L235, G237, and/or P329, EU numbering according to Kabat. In some embodiments, the hIgG1 Fc region comprises the following amino acid substitutions L235A and/or G237A, EU numbering according to Kabat. In some embodiments, the hIgG1 Fc region comprises the following amino acid substitutions L235A, G237A, and P329G, EU numbering according to Kabat. In some embodiments, the hIgG1 Fc region comprises the following amino acid substitutions L235A, G237A, and P329A, EU numbering according to Kabat.

The amino acid sequence of exemplary modified hIg Fc regions that are known in the art to exhibit a decrease in one more effector function is provided in Table 12. The amino acid sequence of several of the hIg Fc regions presented in Table 12 incorporate one or more of the amino acid substitutions set forth above in Table 8 (as indicated in the description). The numbering of amino acid residues in Table 12 (in the Description column) is presented in EU numbering according to Kabat.

TABLE 12
The amino acid sequence of exemplary variant hIg Fc Regions.

| | | SEQ ID |
|---|---|---|
| Description | Amino Acid Sequence | NO |
| hIgG1 CH2 Region + | PCPAPEAAGGPSVFLFPPKPKDTLMISRTPEVTCVVVDVSHE | 687 |
| CH3 Region | DPEVKFNWYVDGVEVHNAKTKPREEQYNSTYRVVSVLTVLHQ | |
| L234A/L235A | DWLNGKEYKCKVSNKALPAPIEKTISKAKGQPREPQVYTLPP | |
| With C-terminal Lysine | SRDELTKNQVSLTCLVKGFYPSDIAVEWESNGQPENNYKTTP | |
| | PVLDSDGSFFLYSKLTVDKSRWQQGNVFSCSVMHEALHNHYT | |
| | QKSLSLSPGK | |
| hIgG1 CH2 Region + | PCPAPEAAGGPSVFLFPPKPKDTLMISRTPEVTCVVVDVSHE | 688 |
| CH3 Region | DPEVKFNWYVDGVEVHNAKTKPREEQYNSTYRVVSVLTVLHQ | |
| L234A/L235A | DWLNGKEYKCKVSNKALPAPIEKTISKAKGQPREPQVYTLPP | |
| Without C-terminal | SRDELTKNQVSLTCLVKGFYPSDIAVEWESNGQPENNYKTTP | |
| Lysine | PVLDSDGSFFLYSKLTVDKSRWQQGNVFSCSVMHEALHNHYT | |
| | QKSLSLSPG | |
| hIgG1 Partial Hinge | TCPPCPAPEAAGGPSVFLFPPKPKDTLMISRTPEVTCVVVDV | 689 |
| Region + CH2 Region + | SHEDPEVKFNWYVDGVEVHNAKTKPREEQYNSTYRVVSVLTV | |
| CH3 Region | LHQDWLNGKEYKCKVSNKALPAPIEKTISKAKGQPREPQVYT | |
| L234A/L235A | LPPSRDELTKNQVSLTCLVKGFYPSDIAVEWESNGQPENNYK | |
| With C-terminal Lysine | TTPPVLDSDGSFFLYSKLTVDKSRWQQGNVFSCSVMHEALHN | |
| | HYTQKSLSLSPGK | |
| hIgG1 Partial Hinge | TCPPCPAPEAAGGPSVFLFPPKPKDTLMISRTPEVTCVVVDV | 690 |
| Region + CH2 Region + | SHEDPEVKFNWYVDGVEVHNAKTKPREEQYNSTYRVVSVLTV | |
| CH3 Region | LHQDWLNGKEYKCKVSNKALPAPIEKTISKAKGQPREPQVYT | |
| L234A/L235A | LPPSRDELTKNQVSLTCLVKGFYPSDIAVEWESNGQPENNYK | |
| Without C-terminal | TTPPVLDSDGSFFLYSKLTVDKSRWQQGNVFSCSVMHEALHN | |
| Lysine | HYTQKSLSLSPG | |
| hIgG1 Hinge Region + | EPKSCDKTHTCPPCPAPEAAGGPSVFLFPPKPKDTLMISRTP | 691 |
| CH2 Region + CH3 | EVTCVVVDVSHEDPEVKFNWYVDGVEVHNAKTKPREEQYNST | |
| Region | YRVVSVLTVLHQDWLNGKEYKCKVSNKALPAPIEKTISKAKG | |
| L234A/L235A | QPREPQVYTLPPSRDELTKNQVSLTCLVKGFYPSDIAVEWES | |
| With C-terminal Lysine | NGQPENNYKTTPPVLDSDGSFFLYSKLTVDKSRWQQGNVFSC | |
| | SVMHEALHNHYTQKSLSLSPGK | |
| hIgG1 Hinge Region + | EPKSCDKTHTCPPCPAPEAAGGPSVFLFPPKPKDTLMISRTP | 692 |
| CH2 Region + CH3 | EVTCVVVDVSHEDPEVKFNWYVDGVEVHNAKTKPREEQYNST | |
| Region | YRVVSVLTVLHQDWLNGKEYKCKVSNKALPAPIEKTISKAKG | |
| L234A/L235A | QPREPQVYTLPPSRDELTKNQVSLTCLVKGFYPSDIAVEWES | |
| Without C-terminal | NGQPENNYKTTPPVLDSDGSFFLYSKLTVDKSRWQQGNVFSC | |
| Lysine | SVMHEALHNHYTQKSLSLSPG | |
| hIgG4 CH2 Region + | APEAAGGPSVFLFPPKPKDTLMISRTPEVTCVVVDVSQEDPE | 693 |
| CH3 Region | VQFNWYVDGVEVHNAKTKPREEQFNSTYRVVSVLTVLHQDWL | |
| S228P/F234A/L235A | NGKEYKCKVSNKGLPSSIEKTISKAKGQPREPQVYTLPPSQE | |
| With C-terminal Lysine | EMTKNQVSLTCLVKGFYPSDIAVEWESNGQPENNYKTTPPVL | |
| | DSDGSFFLYSRLTVDKSRWQEGNVFSCSVMHEALHNHYTQKS | |
| | LSLSLGK | |
| hIgG4 CH2 Region + | APEAAGGPSVFLFPPKPKDTLMISRTPEVTCVVVDVSQEDPE | 694 |
| CH3 Region | VQFNWYVDGVEVHNAKTKPREEQFNSTYRVVSVLTVLHQDWL | |
| S228P/F234A/L235A | NGKEYKCKVSNKGLPSSIEKTISKAKGQPREPQVYTLPPSQE | |
| Without C-terminal | EMTKNQVSLTCLVKGFYPSDIAVEWESNGQPENNYKTTPPVL | |
| Lysine | DSDGSFFLYSRLTVDKSRWQEGNVFSCSVMHEALHNHYTQKS | |
| | LSLSLG | |
| hIgG4 Partial Hinge | PCPSCPAPEAAGGPSVFLFPPKPKDTLMISRTPEVTCVVVDV | 695 |
| Region + CH2 Region + | SQEDPEVQFNWYVDGVEVHNAKTKPREEQFNSTYRVVSVLTV | |
| CH3 Region | LHQDWLNGKEYKCKVSNKGLPSSIEKTISKAKGQPREPQVYT | |
| S228P/F234A/L235A | LPPSQEEMTKNQVSLTCLVKGFYPSDIAVEWESNGQPENNYK | |
| With C-terminal Lysine | TTPPVLDSDGSFFLYSRLTVDKSRWQEGNVFSCSVMHEALHN | |
| | HYTQKSLSLSLGK | |
| hIgG4 Partial Hinge | PCPPCPAPEAAGGPSVFLFPPKPKDTLMISRTPEVTCVVVDV | 696 |
| Region + CH2 Region + | SQEDPEVQFNWYVDGVEVHNAKTKPREEQFNSTYRVVSVLTV | |
| CH3 Region | LHQDWLNGKEYKCKVSNKGLPSSIEKTISKAKGQPREPQVYT | |
| S228P/F234A/L235A | LPPSQEEMTKNQVSLTCLVKGFYPSDIAVEWESNGQPENNYK | |
| Without C-terminal | TTPPVLDSDGSFFLYSRLTVDKSRWQEGNVFSCSVMHEALHN | |
| Lysine | HYTQKSLSLSLG | |
| hIgG4 Hinge Region + | ESKYGPPCPPCPAPEAAGGPSVFLFPPKPKDTLMISRTPEVT | 697 |
| CH2 Region + CH3 | CVVVDVSQEDPEVQFNWYVDGVEVHNAKTKPREEQFNSTYRV | |
| Region | VSVLTVLHQDWLNGKEYKCKVSNKGLPSSIEKTISKAKGQPR | |
| S228P/F234A/L235A | EPQVYTLPPSQEEMTKNQVSLTCLVKGFYPSDIAVEWESNGQ | |
| With C-terminal Lysine | PENNYKTTPPVLDSDGSFFLYSRLTVDKSRWQEGNVFSCSVM | |
| | HEALHNHYTQKSLSLSLGK | |
| hIgG4 Hinge Region + | ESKYGPPCPPCPAPEAAGGPSVFLFPPKPKDTLMISRTPEVT | 698 |
| CH2 Region + CH3 | CVVVDVSQEDPEVQFNWYVDGVEVHNAKTKPREEQFNSTYRV | |
| Region | VSVLTVLHQDWLNGKEYKCKVSNKGLPSSIEKTISKAKGQPR | |
| S228P/F234A/L235A | EPQVYTLPPSQEEMTKNQVSLTCLVKGFYPSDIAVEWESNGQ | |
| Without C-terminal | PENNYKTTPPVLDSDGSFFLYSRLTVDKSRWQEGNVFSCSVM | |
| Lysine | HEALHNHYTQKSLSLSLG | |
| hIgG4 Hinge Region + | AESKYGPPCPPCPAPEAAGGPSVFLFPPKPKDTLMISRTPEV | 699 |
| CH2 Region + CH3 | TCVVVDVSQEDPEVQFNWYVDGVEVHNAKTKPREEQFNSTYR | |
| Region | VVSVLTVLHQDWLNGKEYKCKVSNKGLPSSIEKTISKAKGQP | |
| (Variant) | REPQVYTLPPSQEEMTKNQVSLTCLVKGFYPSDIAVEWESNG | |
| S228P/F234A/L235A | QPENNYKTTPPVLDSDGSFFLYSRLTVDKSRWQEGNVFSCSV | |
| With C-terminal Lysine | MHEALHNHYTQKSLSLSLGK | |
| hIgG4 Hinge Region + | AESKYGPPCPPCPAPEAAGGPSVFLFPPKPKDTLMISRTPEV | 700 |
| CH2 Region + CH3 | TCVVVDVSQEDPEVQFNWYVDGVEVHNAKTKPREEQFNSTYR | |
| Region | VVSVLTVLHQDWLNGKEYKCKVSNKGLPSSIEKTISKAKGQP | |
| (Variant) | REPQVYTLPPSQEEMTKNQVSLTCLVKGFYPSDIAVEWESNG | |
| S228P/F234A/L235A | QPENNYKTTPPVLDSDGSFFLYSRLTVDKSRWQEGNVFSCSV | |
| Without C-terminal | MHEALHNHYTQKSLSLSLG | |
| Lysine | | |

In some embodiments, a fusion protein (e.g., described herein) comprises a modified Ig Fc region.

In some embodiments, the modified hIg Fc region comprises one or more sets of amino acid substitutions set forth in Table 8 and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12. In some embodiments, the modified hIg Fc region comprises one or more sets of amino acid substitutions set forth in Table 8 and comprises an amino acid sequence at least 90% identical to the amino acid sequence of a polypeptide set forth in Table 12. In some embodiments, the modified hIg Fc region comprises one or more sets of amino acid substitutions set forth in Table 8 and comprises an amino acid sequence at least 95% identical to the amino acid sequence of a polypeptide set forth in Table 12. In some embodiments, the modified hIg Fc region comprises one or more sets of amino acid substitutions set forth in Table 8 and comprises an amino acid sequence at least 99% identical to the amino acid sequence of a polypeptide set forth in Table 12.

In some embodiments, the modified hIg Fc region comprises one or more sets of amino acid substitutions set forth in Table 8 and comprises an amino acid sequence 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12. In some embodiments, the modified hIg Fc region comprises one or more sets of amino acid substitutions set forth in Table 8 and comprises an amino acid sequence 90% identical to the amino acid sequence of a polypeptide set forth in Table 12. In some embodiments, the modified hIg Fc region comprises one or more sets of amino acid substitutions set forth in Table 8 and comprises an amino acid sequence 95% identical to the amino acid sequence of a polypeptide set forth in Table 12. In some embodiments, the modified hIg Fc region comprises one or more sets of amino acid substitutions set forth in Table 8 and comprises an amino acid sequence 99% identical to the amino acid sequence of a polypeptide set forth in Table 12. In some embodiments, the modified hIg Fc region comprises one or more sets of amino acid substitutions set forth in Table 8 and comprises an amino acid sequence 100% identical to the amino acid sequence of a polypeptide set forth in Table 12.

In some embodiments, the modified hIg Fc region comprises one or more sets of amino acid substitutions set forth in Table 8 and consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12. In some embodiments, the modified hIg Fc region comprises one or more sets of amino acid substitutions set forth in Table 8 and consists of an amino acid sequence at least 90% identical to the amino acid sequence of a polypeptide set forth in Table 12. In some embodiments, the modified hIg Fc region comprises one or more sets of amino acid substitutions set forth in Table 8 and consists of an amino acid sequence at least 95% identical to the amino acid sequence of a polypeptide set forth in Table 12. In some embodiments, the modified hIg Fc region comprises one or more sets of amino acid substitutions set forth in Table 8 and consists of an amino acid sequence at least 99% identical to the amino acid sequence of a polypeptide set forth in Table 12.

In some embodiments, the modified hIg Fc region comprises one or more sets of amino acid substitutions set forth in Table 8 and consists of an amino acid sequence 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12. In some embodiments, the modified hIg Fc region comprises one or more sets of amino acid substitutions set forth in Table 8 and consists of an amino acid sequence 90% identical to the amino acid sequence of a polypeptide set forth in Table 12. In some embodiments, the modified hIg Fc region comprises one or more sets of amino acid substitutions set forth in Table 8 and consists of an amino acid sequence 95% identical to the amino acid sequence of a polypeptide set forth in Table 12. In some embodiments, the modified hIg Fc region comprises one or more sets of amino acid substitutions set forth in Table 8 and consists of an amino acid sequence 99% identical to the amino acid sequence of a polypeptide set forth in Table 12. In some embodiments, the modified hIg Fc region comprises one or more sets of amino acid substitutions set forth in Table 8 and consists of an amino acid sequence 100% identical to the amino acid sequence of a polypeptide set forth in Table 12.

In some embodiments, the modified hIg Fc region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12. In some embodiments, the modified hIg Fc region comprises an amino acid sequence at least 90% identical to the amino acid sequence of a polypeptide set forth in Table 12. In some embodiments, the modified hIg Fc region comprises an amino acid sequence at least 95% identical to the amino acid sequence of a polypeptide set forth in Table 12. In some embodiments, the modified hIg Fc region comprises an amino acid sequence at least 99% identical to the amino acid sequence of a polypeptide set forth in Table 12.

In some embodiments, the modified hIg Fc region comprises an amino acid sequence 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12. In some embodiments, the modified hIg Fc region comprises an amino acid sequence 90% identical to the amino acid sequence of a polypeptide set forth in Table 12. In some embodiments, the modified hIg Fc region comprises an amino acid sequence 95% identical to the amino acid sequence of a polypeptide set forth in Table 12. In some embodiments, the modified hIg Fc region comprises an amino acid sequence 99% identical to the amino acid sequence of a polypeptide set forth in Table 12. In some embodiments, the modified hIg Fc comprises an amino acid sequence 100% identical to the amino acid sequence of a polypeptide set forth in Table 12.

In some embodiments, the modified hIg Fc region consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12. In some embodiments, the modified hIg Fc region consists of an amino acid sequence at least 90% identical to the amino acid sequence of a polypeptide set forth in Table 12. In some embodiments, the modified hIg Fc region consists of an amino acid sequence at least 95% identical to the amino acid sequence of a polypeptide set forth in Table 12. In some embodiments, the modified hIg Fc region consists of an amino acid sequence at least 99% identical to the amino acid sequence of a polypeptide set forth in Table 12.

In some embodiments, the modified hIg Fc region consists of an amino acid sequence 85%, 86%, 87%, 88%, 89%, 90%, 91%, 92%, 93%, 94%, 95%, 96%, 97%, 98%, 99%, or 100% identical to the amino acid sequence of a polypeptide set forth in Table 12. In some embodiments, the modified hIg Fc region consists of an amino acid sequence 90% identical to the amino acid sequence of a polypeptide set forth in Table 12. In some embodiments, the modified hIg Fc region consists of an amino acid sequence 95% identical to the amino acid sequence of a polypeptide set forth in Table 12. In some embodiments, the modified hIg Fc region consists of an amino acid sequence 99% identical to the amino acid sequence of a polypeptide set forth in Table 12. In some embodiments, the modified hIg Fc region consists of an amino acid sequence 100% identical to the amino acid sequence of a polypeptide set forth in Table 12.

In some embodiments, the amino acid sequence of the modified hIg Fc region comprises one or more sets of amino acid substitutions set forth in Table 8 and comprises the amino acid sequence of a polypeptide set forth in Table 12, and further comprises 1 or more but less than 15% (less than 12%, less than 10%, less than 8%), amino acid variations (e.g., amino acid substitutions, deletions, or additions). In some embodiments, the amino acid sequence of the modified hIg Fc region comprises one or more sets of amino acid substitutions set forth in Table 8 and comprises the amino acid sequence of a polypeptide set forth in Table 12, and further comprises at least about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and comprises the amino acid sequence of a polypeptide set forth in Table 12, and further comprises about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and comprises the amino acid sequence of a polypeptide set forth in Table 12, and further consists of about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and comprises the amino acid sequence of a polypeptide set forth in Table 12, and further comprises no more than about 1, 2, 3, 4, 5, 6, 7, 8, 9, or 10 amino acid variations (e.g., substitutions, additions, deletions, etc.).

In some embodiments, the amino acid sequence of the modified hIg Fc region comprises one or more sets of amino acid substitutions set forth in Table 8 and consists of the amino acid sequence of a polypeptide set forth in Table 12, and further comprises 1 or more but less than 15% (less than 12%, less than 10%, less than 8%), amino acid variations (e.g., amino acid substitutions, deletions, or additions). In some embodiments, the amino acid sequence of the modified hIg Fc region comprises one or more sets of amino acid substitutions set forth in Table 8 and consists of the amino acid sequence of a polypeptide set forth in Table 12, and further comprises at least about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and consists of the amino acid sequence of a polypeptide set forth in Table 12, and further comprises about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and consists of the amino acid sequence of a polypeptide set forth in Table 12, and further consists of about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and consists of the amino acid sequence of a polypeptide set forth in Table 12, and further comprises no more than about 1, 2, 3, 4, 5, 6, 7, 8, 9, or 10 amino acid variations (e.g., substitutions, additions, deletions, etc.).

In some embodiments, the amino acid sequence of the modified hIg Fc region comprises the amino acid sequence of a polypeptide set forth in Table 12, and further comprises 1 or more but less than 15% (less than 12%, less than 10%, less than 8%), amino acid variations (e.g., amino acid substitutions, deletions, or additions). In some embodiments, the amino acid sequence of the modified hIg Fc region comprises the amino acid sequence of a polypeptide set forth in Table 12, and further comprises at least about 1, 2, 3, 4, 5, 6, 7, 8, 9, or 10 amino acid variations (e.g., substitutions, additions, deletions, etc.). In some embodiments, the amino acid sequence of the modified hIg Fc region comprises the amino acid sequence of a polypeptide set forth in Table 12, and further comprises about 1, 2, 3, 4, 5, 6, 7, 8, 9, or 10 amino acid variations (e.g., substitutions, additions, deletions, etc.). In some embodiments, the amino acid sequence of the modified hIg Fc region comprises the amino acid sequence of a polypeptide set forth in Table 12, and further consists of about 1, 2, 3, 4, 5, 6, 7, 8, 9, or 10 amino acid variations (e.g., substitutions, additions, deletions, etc.). In some embodiments, the amino acid sequence of the modified hIg Fc region comprises the amino acid sequence of a polypeptide set forth in Table 12, and further comprises no more than about 1, 2, 3, 4, 5, 6, 7, 8, 9, or 10 amino acid variations (e.g., substitutions, additions, deletions, etc.).

In some embodiments, the amino acid sequence of the modified hIg Fc region consists of the amino acid sequence of a polypeptide set forth in Table 12, and further comprises 1 or more but less than 15% (less than 12%, less than 10%, less than 8%), amino acid variations (e.g., amino acid substitutions, deletions, or additions). In some embodiments, the amino acid sequence of the modified hIg Fc region consists of the amino acid sequence of a polypeptide set forth in Table 12, and further comprises at least about 1, 2, 3, 4, 5, 6, 7, 8, 9, or 10 amino acid variations (e.g., substitutions, additions, deletions, etc.). In some embodiments, the amino acid sequence of the modified hIg Fc region consists of the amino acid sequence of a polypeptide set forth in Table 12, and further comprises about 1, 2, 3, 4, 5, 6, 7, 8, 9, or 10 amino acid variations (e.g., substitutions, additions, deletions, etc.). In some embodiments, the amino acid sequence of the modified hIg Fc region 8 consists of the amino acid sequence of a polypeptide set forth in Table 12, and further consists of about 1, 2, 3, 4, 5, 6, 7, 8, 9, or 10 amino acid variations (e.g., substitutions, additions, deletions, etc.). In some embodiments, the amino acid sequence of the modified hIg Fc region consists of the amino acid sequence of a polypeptide set forth in Table 12, and further comprises no more than about 1, 2, 3, 4, 5, 6, 7, 8, 9, or 10 amino acid variations (e.g., substitutions, additions, deletions, etc.).

In some embodiments, the modified hIg Fc region comprises one or more sets of amino acid substitutions set forth in Table 8 and comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in any one of SEQ ID NOS: 687-700. In some embodiments, the modified hIg Fc region comprises one or more sets of amino acid substitutions set forth in Table 8 and comprises an amino acid sequence at least 90% identical to the amino acid sequence of a polypeptide set forth in any one of SEQ ID NOS: 687-700. In some embodiments, the modified hIg Fc region comprises one or more sets of amino acid substitutions set forth in Table 8 and comprises an amino acid sequence at least 95% identical to the amino acid sequence of a polypeptide set forth in any one of SEQ ID NOS: 687-700. In some embodiments, the modified hIg Fc region comprises one or more sets of amino acid substitutions set forth in Table 8 and comprises an amino acid sequence at least 99% identical to the amino acid sequence of a polypeptide set forth in any one of SEQ ID NOS: 687-700.

In some embodiments, the modified hIg Fc region comprises one or more sets of amino acid substitutions set forth in Table 8 and comprises an amino acid sequence 85%, 86%, 87%, 88%, 89%, 90%, 91%, 92%, 93%, 94%, 95%, 96%, 97%, 98%, 99%, or 100% identical to the amino acid sequence of a polypeptide set forth in any one of SEQ ID NOS: 687-700. In some embodiments, the modified hIg Fc region comprises one or more sets of amino acid substitutions set forth in Table 8 and comprises an amino acid sequence 90% identical to the amino acid sequence of a polypeptide set forth in any one of SEQ ID NOS: 687-700. In some embodiments, the modified hIg Fc region comprises one or more sets of amino acid substitutions set forth in Table 8 and comprises an amino acid sequence 95% identical to the amino acid sequence of a polypeptide set forth in any one of SEQ ID NOS: 687-700. In some embodiments, the modified hIg Fc region comprises one or more sets of amino acid substitutions set forth in Table 8 and comprises an amino acid sequence 99% identical to the amino acid sequence of a polypeptide set forth in any one of SEQ ID NOS: 687-700. In some embodiments, the modified hIg Fc region comprises one or more sets of amino acid substitutions set forth in Table 8 and comprises an amino acid sequence 100% identical to the amino acid sequence of a polypeptide set forth in any one of SEQ ID NOS: 687-700.

In some embodiments, the modified hIg Fc region comprises one or more sets of amino acid substitutions set forth in Table 8 and consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in any one of SEQ ID NOS: 687-700. In some embodiments, the modified hIg Fc region comprises one or more sets of amino acid substitutions set forth in Table 8 and consists of an amino acid sequence at least 90% identical to the amino acid sequence of a polypeptide set forth in any one of SEQ ID NOS: 687-700. In some embodiments, the modified hIg Fc region comprises one or more sets of amino acid substitutions set forth in Table 8 and consists of an amino acid sequence at least 95% identical to the amino acid sequence of a polypeptide set forth in any one of SEQ ID NOS: 687-700. In some embodiments, the modified hIg Fc region comprises one or more sets of amino acid substitutions set forth in Table 8 and consists of an amino acid sequence at least 99% identical to the amino acid sequence of a polypeptide set forth in any one of SEQ ID NOS: 687-700.

In some embodiments, the modified hIg Fc region comprises one or more sets of amino acid substitutions set forth in Table 8 and consists of an amino acid sequence 85%, 86%, 87%, 88%, 89%, 90%, 91%, 92%, 93%, 94%, 95%, 96%, 97%, 98%, 99%, or 100% identical to the amino acid sequence of a polypeptide set forth in any one of SEQ ID NOS: 687-700. In some embodiments, the modified hIg Fc region comprises one or more sets of amino acid substitutions set forth in Table 8 and consists of an amino acid sequence 90% identical to the amino acid sequence of a polypeptide set forth in any one of SEQ ID NOS: 687-700. In some embodiments, the modified hIg Fc region comprises one or more sets of amino acid substitutions set forth in Table 8 and consists of an amino acid sequence 95% identical to the amino acid sequence of a polypeptide set forth in any one of SEQ ID NOS: 687-700. In some embodiments, the modified hIg Fc region comprises one or more sets of amino acid substitutions set forth in Table 8 and consists of an amino acid sequence 99% identical to the amino acid sequence of a polypeptide set forth in any one of SEQ ID NOS: 687-700. In some embodiments, the modified hIg Fc region comprises one or more sets of amino acid substitutions set forth in Table 8 and consists of an amino acid sequence 100% identical to the amino acid sequence of a polypeptide set forth in any one of SEQ ID NOS: 687-700.

In some embodiments, the modified hIg Fc region comprises an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in any one of SEQ ID NOS: 687-700. In some embodiments, the modified hIg Fc region comprises an amino acid sequence at least 90% identical to the amino acid sequence of a polypeptide set forth in any one of SEQ ID NOS: 687-700. In some embodiments, the modified hIg Fc region comprises an amino acid sequence at least 95% identical to the amino acid sequence of a polypeptide set forth in any one of SEQ ID NOS: 687-700. In some embodiments, the modified hIg Fc region comprises an amino acid sequence at least 99% identical to the amino acid sequence of a polypeptide set forth in any one of SEQ ID NOS: 687-700.

In some embodiments, the modified hIg Fc region comprises an amino acid sequence 85%, 86%, 87%, 88%, 89%, 90%, 91%, 92%, 93%, 94%, 95%, 96%, 97%, 98%, 99%, or 100% identical to the amino acid sequence of a polypeptide set forth in any one of SEQ ID NOS: 687-700. In some embodiments, the modified hIg Fc region comprises an amino acid sequence 90% identical to the amino acid sequence of a polypeptide set forth in any one of SEQ ID NOS: 687-700. In some embodiments, the modified hIg Fc region comprises an amino acid sequence 95% identical to the amino acid sequence of a polypeptide set forth in any one of SEQ ID NOS: 687-700. In some embodiments, the modified hIg Fc region comprises an amino acid sequence 99% identical to the amino acid sequence of a polypeptide set forth in any one of SEQ ID NOS: 687-700. In some embodiments, the modified hIg Fc comprises an amino acid sequence 100% identical to the amino acid sequence of a polypeptide set forth in any one of SEQ ID NOS: 687-700.

In some embodiments, the modified hIg Fc region consists of an amino acid sequence at least 85%, 86%, 87%, 88%, 89%, 90%, 91%, 92%, 93%, 94%, 95%, 96%, 97%, 98%, 99%, or 100% identical to the amino acid sequence of a polypeptide set forth in any one of SEQ ID NOS: 687-700. In some embodiments, the modified hIg Fc region consists of an amino acid sequence at least 90% identical to the amino acid sequence of a polypeptide set forth in any one of SEQ ID NOS: 687-700. In some embodiments, the modified hIg Fc region consists of an amino acid sequence at least 95% identical to the amino acid sequence of a polypeptide set forth in any one of SEQ ID NOS: 687-700. In some embodiments, the modified hIg Fc region consists of an amino acid sequence at least 99% identical to the amino acid sequence of a polypeptide set forth in any one of SEQ ID NOS: 687-700.

In some embodiments, the modified hIg Fc region consists of an amino acid sequence 85%, 86%, 87%, 88%, 89%, 90%, 91%, 92%, 93%, 94%, 95%, 96%, 97%, 98%, 99%, or 100% identical to the amino acid sequence of a polypeptide set forth in any one of SEQ ID NOS: 687-700. In some embodiments, the modified hIg Fc region consists of an amino acid sequence 90% identical to the amino acid sequence of a polypeptide set forth in any one of SEQ ID NOS: 687-700. In some embodiments, the modified hIg Fc region consists of an amino acid sequence 95% identical to the amino acid sequence of a polypeptide set forth in any one of SEQ ID NOS: 687-700. In some embodiments, the modified hIg Fc region consists of an amino acid sequence 99% identical to the amino acid sequence of a polypeptide set forth in any one of SEQ ID NOS: 687-700. In some embodiments, the modified hIg Fc region consists of an amino acid sequence 100% identical to the amino acid sequence of a polypeptide set forth in any one of SEQ ID NOS: 687-700.

In some embodiments, the amino acid sequence of the modified hIg Fc region comprises one or more sets of amino acid substitutions set forth in Table 8 and comprises the amino acid sequence of a polypeptide set forth in any one of SEQ ID NOS: 687-700, and further comprises 1 or more but less than 15% (less than 12%, less than 10%, less than 8%), amino acid variations (e.g., amino acid substitutions, deletions, or additions). In some embodiments, the amino acid sequence of the modified hIg Fc region comprises one or more sets of amino acid substitutions set forth in Table 8 and comprises the amino acid sequence of a polypeptide set forth in any one of SEQ ID NOS: 687-700, and further comprises at least about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and comprises the amino acid sequence of a polypeptide set forth in any one of SEQ ID NOS: 687-700, and further comprises about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and comprises the amino acid sequence of a polypeptide set forth in any one of SEQ ID NOS: 687-700, and further consists of about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and comprises the amino acid sequence of a polypeptide set forth in any one of SEQ ID NOS: 687-700, and further comprises no more than about 1, 2, 3, 4, 5, 6, 7, 8, 9, or 10 amino acid variations (e.g., substitutions, additions, deletions, etc.).

In some embodiments, the amino acid sequence of the modified hIg Fc region comprises one or more sets of amino acid substitutions set forth in Table 8 and consists of the amino acid sequence of a polypeptide set forth in any one of SEQ ID NOS: 687-700, and further comprises 1 or more but less than 15% (less than 12%, less than 10%, less than 8%), amino acid variations (e.g., amino acid substitutions, deletions, or additions). In some embodiments, the amino acid sequence of the modified hIg Fc region comprises one or more sets of amino acid substitutions set forth in Table 8 and consists of the amino acid sequence of a polypeptide set forth in any one of SEQ ID NOS: 687-700, and further comprises at least about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and consists of the amino acid sequence of a polypeptide set forth in any one of SEQ ID NOS: 687-700, and further comprises about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and consists of the amino acid sequence of a polypeptide set forth in any one of SEQ ID NOS: 687-700, and further consists of about 1, 2, 3, 4, 5, 6, 7, 8, 9, or 10 amino acid variations (e.g., substitutions, additions, deletions, etc.). In some embodiments, the amino acid sequence of the modified hIg Fc region comprises one or more sets of amino acid substitutions set forth in Table 8 and consists of the amino acid sequence of a polypeptide set forth in any one of SEQ ID NOS: 687-700, and further comprises no more than about 1, 2, 3, 4, 5, 6, 7, 8, 9, or 10 amino acid variations (e.g., substitutions, additions, deletions, etc.).

In some embodiments, the amino acid sequence of the modified hIg Fc region comprises the amino acid sequence of a polypeptide set forth in any one of SEQ ID NOS: 687-700, and further comprises 1 or more but less than 15% (less than 12%, less than 10%, less than 8%), amino acid variations (e.g., amino acid substitutions, deletions, or additions). In some embodiments, the amino acid sequence of the modified hIg Fc region comprises the amino acid sequence of a polypeptide set forth in any one of SEQ ID NOS: 687-700, and further comprises at least about 1, 2, 3, 4, 5, 6, 7, 8, 9, or 10 amino acid variations (e.g., substitutions, additions, deletions, etc.). In some embodiments, the amino acid sequence of the modified hIg Fc region comprises the amino acid sequence of a polypeptide set forth in any one of SEQ ID NOS: 687-700, and further comprises about 1, 2, 3, 4, 5, 6, 7, 8, 9, or 10 amino acid variations (e.g., substitutions, additions, deletions, etc.). In some embodiments, the amino acid sequence of the modified hIg Fc region comprises the amino acid sequence of a polypeptide set forth in any one of SEQ ID NOS: 687-700, and further consists of about 1, 2, 3, 4, 5, 6, 7, 8, 9, or 10 amino acid variations (e.g., substitutions, additions, deletions, etc.). In some embodiments, the amino acid sequence of the modified hIg Fc region comprises the amino acid sequence of a polypeptide set forth in any one of SEQ ID NOS: 687-700, and further comprises no more than about 1, 2, 3, 4, 5, 6, 7, 8, 9, or 10 amino acid variations (e.g., substitutions, additions, deletions, etc.).

In some embodiments, the amino acid sequence of the modified hIg Fc region consists of the amino acid sequence of a polypeptide set forth in any one of SEQ ID NOS: 687-700, and further comprises 1 or more but less than 15% (less than 12%, less than 10%, less than 8%), amino acid variations (e.g., amino acid substitutions, deletions, or additions). In some embodiments, the amino acid sequence of the modified hIg Fc region consists of the amino acid sequence of a polypeptide set forth in any one of SEQ ID NOS: 687-700, and further comprises at least about 1, 2, 3, 4, 5, 6, 7, 8, 9, or 10 amino acid variations (e.g., substitutions, additions, deletions, etc.). In some embodiments, the amino acid sequence of the modified hIg Fc region consists of the amino acid sequence of a polypeptide set forth in any one of SEQ ID NOS: 687-700, and further comprises about 1, 2, 3, 4, 5, 6, 7, 8, 9, or 10 amino acid variations (e.g., substitutions, additions, deletions, etc.). In some embodiments, the amino acid sequence of the modified hIg Fc region 8 consists of the amino acid sequence of a polypeptide set forth in any one of SEQ ID NOS: 687-700, and further consists of about 1, 2, 3, 4, 5, 6, 7, 8, 9, or 10 amino acid variations (e.g., substitutions, additions, deletions, etc.). In some embodiments, the amino acid sequence of the modified hIg Fc region consists of the amino acid sequence of a polypeptide set forth in any one of SEQ ID NOS: 687-700, and further comprises no more than about 1, 2, 3, 4, 5, 6, 7, 8, 9, or 10 amino acid variations (e.g., substitutions, additions, deletions, etc.).

### 5.4.4.2 Enhanced Ig Effector Function

In some embodiments, the Ig Fc region exhibits an enhancement (e.g., an increase) in one or more Fc effector function relative to a reference (e.g., wild type) Ig Fc region. Exemplary Ig Fc effector functions include, but are not limited to, ADCC, ADCP, CDC, binding affinity to C1q, and binding affinity to one or more human Fc receptor (e.g., an Fc receptor (e.g., (e.g., FcγRI, FcγRIIa, FcγRIIIa, and/or FcγRIIIb). In some embodiments, the Fc region exhibits one or more enhanced Fc effector function, relative to a reference Ig (e.g., hIg, mIg).

In some embodiments, the hIg Fc region is modified (e.g., comprises one or more variation (e.g., one or more amino acid substitution, deletion, addition, etc.); altered glycosylation (e.g., afucosylation))) (referred to herein as a “modified hIg Fc”). In some embodiments, the modification (e.g., the variation (e.g., one or more amino acid substitution, deletion, addition, etc.); altered glycosylation (e.g., afucosylation))) enhances (e.g., increases) one or more Fc effector function, relative to a reference hIg Fc that does not comprise the modification (e.g., the one or more variation (e.g., the one or more amino acid substitution, deletion, addition, etc.; the altered glycosylation (e.g., afucosylation))).

In some embodiments, the modified Ig (e.g., hIg, mIg) Fc fusion protein exhibits enhanced ADCC compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced CDC compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced ADCP compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits enhanced binding affinity to one or more Fc receptor (e.g., human Fc receptor) (e.g., an Fcγ receptor (e.g., FcγRI, FcγRIIa, FcγRIIIa, and/or FcγRIIIb) compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRI, FcγRIIa, FcγRIIIa, and/or FcγRIIIb compared to a reference fusion protein that does not comprise the hIg Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRI compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRIIa compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRIIIa compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits enhanced binding affinity to FcγRIIIb compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits reduced binding affinity to one or more Fc receptor (e.g., human Fc receptor) ((e.g., an Fcγ receptor (e.g., FcγRIIb)) compared to a reference fusion protein that does not comprise the Ig (e.g., hIg, mIg) Fc modification (e.g., the one or more variation (e.g., one or more amino acid substitution, deletion, or addition)). In some embodiments, the modified Ig (e.g., hIg, mIg) Fc fusion protein exhibits reduced binding affinity to FcγRIIb compared to a reference fusion protein that does not comprise the Ig (e.g., hIg, mIg) Fc modification (e.g., the one or more variation (e.g., one or more amino acid substitution, deletion, or addition)).

In some embodiments, the modified Ig (e.g., hIg, mIg) Fc fusion protein exhibits enhanced binding affinity to C1q compared to a reference fusion protein that does not comprise the Ig (e.g., hIg, mIg) Fc modification (e.g., the one or more variation (e.g., one or more amino acid substitution, deletion, or addition)).

Amino acid substitutions and glycoengineering that enhance (e.g., increase) one or more hIg Fc effector function are known in the art. See for example, Liu R, Oldham R J, Teal E, Beers S A, Cragg M S. Fc-Engineering for Modulated Effector Functions-Improving Antibodies for Cancer Treatment. Antibodies (Basel). 2020; 9(4):64. Published 2020 Nov. 17. doi:10.3390/antib9040064; van der Horst H J, Nijhof I S, Mutis T, Chamuleau M E D. Fc-Engineered Antibodies with Enhanced Fc-Effector Function for the Treatment of B-Cell Malignancies. Cancers (Basel). 2020; 12(10):3041. Published 2020 Oct. 19. Doi:10.3390/cancers12103041; and Saunders Kevin, “Conceptual Approaches to Modulating Antibody Effector Functions and Circulation Half-Life,” Frontiers in Immunology, v10 (Jun. 7, 2019) DOI=10.3389/fimmu.2019.01296, the full contents of each of which is incorporated by reference herein for all purposes.

Table 13 below, provides exemplary amino acid substitutions (and combinations thereof) and glycoengineering that can be utilized to increase one or more hIg Fc effector function. The numbering of amino acid residues in Table 13 is presented in EU numbering according to Kabat. The effects on effector function set forth in Table 8 are exemplary only and not intended to be limiting. The amino acid substitutions set forth in Table 8 are with reference to an IgG1 Fc region (except where noted). However, a person of ordinary skill in the could identify the corresponding amino acid in a non-IgG1 Fc region, for example in an IgG2 or IgG4 Fc region, should the base amino acid be different between the IgG1 and non-IgG1 Fc region.

TABLE 13
Exemplary hIg Fc Variations and Glycoengineering
to Increase Ig Fc Effector Function.

| | |
|---|---|
| Variation/ | Exemplary Effects on |
| Glycoengineering | Effector Function |

Amino Acid Substitutions

| S298A/E333A/K334A | | Increases ADCC |
|---|---|---|
| S239D/I332E | | Increases ADCC |
| P247I/A339Q | | Increases ADCC |
| S239D/A330L/I332E | | Increases ADCC |
| G236A/S239D/I332E | | Increases ADCC |
| F243L/R292P/Y300L/V305I/P396L | | Increases ADCC |
| L235V/F243L/R292P/Y300L/P396L | | Increases ADCC |
| One Heavy | Opposing Heavy | Increases ADCC |
| Chain: L234Y/ | Chain: D270E/ | |
| L235Q/G236W/ | K326D/A330M/ | |
| S239M/H268D/ | K334E | |
| D270E/S298A | | |

| | |
|---|---|
| F243L/R292P/Y300L/V305I/P396L | Increases ADCP |
| S239D/I332E/A330L | Increases ADCP |
| S239D/I332E/A330L/G236A | Increases ADCP |
| S239D/I332E/G326A | Increases ADCP |
| G236A | Increases ADCP |
| G236A/S239D/I332E | Increases ADCP |
| S239D/I332E | Increases ADCP |
| K326W/E333S | Increases C1q Binding and CDC |
| S267E/H268E/S324T | Increases C1q Binding and CDC |
| S298A/E333A/K334A | Enhances FcγRIIIa binding |
| S239D/I332E | Enhances FcγRIIIa binding |
| P247I/A339Q | Enhances FcγRIIIa binding |
| F243L/R292P/Y300L/V305I/P396L | Enhances FcγRIIa binding; |
| | decreases FcβRIIb binding |
| G236A | Enhances FcγRIIa binding |

Glycoengineering

| | |
|---|---|
| Afucosylation | Increases ADCC, Increases ADCP |
| Galactosylation | Increases CDC |

In some embodiments, the Ig Fc region is a hIg Fc region. In some embodiments, the hIg Fe (e.g., IgG1 Fc) region comprises any one or more of the amino acid substitutions set forth in Table 13 (i.e., any one or more amino acid substitution set forth in any set of amino acid substitutions set forth in Table 13). In some embodiments, the hIg Fe (e.g., IgG1 Fc) comprises any one or more of the sets of amino acid substitutions set forth in Table 13. In some embodiments, the hIg Fe (e.g., IgG1 Fc) region comprises any one or more of the glycosylation changes set forth in Table 13.

In some embodiments, a fusion protein (e.g., described herein) comprises a modified hIg Fc region. In some embodiments, the modified hIg Fc region comprises an amino acid substitution at any one or more (e.g., 1, 2, 3, 4, 5, 6, 7, 8, 9, or 10, or more) of amino acid positions S298, E333, K334, S239, I332, P247, A339, A330, G236, F243, R292, Y300, V305, P396, L235, F243, R292, Y300, P396, F243, R292, Y300, V305, P396, K326, E333, S267E, H268, S324, S298, E333, K334, L234, L235, G236, S239, H268, D270, S298 D270, K326, A330, and/or K334. In some embodiments, the modified Ig Fc region comprises an amino acid substitution at from about 1-10 (e.g., 1-9, 1-8, 1-7, 1-6, 1-5, 1-4, 1-3, or 1-2) of the following amino acid positions S298, E333, K334, S239, I332, P247, A339, A330, G236, F243, R292, Y300, V305, P396, L235, F243, R292, Y300, P396, F243, R292, Y300, V305, P396, K326, E333, S267E, H268, S324, S298, E333, K334, L234, L235, G236, S239, H268, D270, S298 D270, K326, A330, and/or K334.

In some embodiments, the modified hIg Fc region comprises one or more (e.g., 1, 2, 3, 4, 5, 6, 7, 8, 9, or 10, or more) of the following amino acid substitutions S298A, E333A, K334A, S239D, I332E, P247I, A339Q, A330L, G236A, F243L, R292P, Y300L, V305I, P396L, L235V, F243L, R292P, Y300L, P396L, F243L, R292P, Y300L, V305I, P396L, K326W, E333S, S267E, H268E, S324T, S298A, E333A, K334A, L234Y, L235Q, G236W, S239M, H268D, D270E, S298A D270E, K326D, A330M, and/or K334E.

In some embodiments, the modified hIg Fc region comprises from about 1-10 (e.g., 1-9, 1-8, 1-7, 1-6, 1-5, 1-4, 1-3, or 1-2) of the following amino acid substitutions S298A, E333A, K334A, S239D, I332E, P247I, A339Q, A330L, G236A, F243L, R292P, Y300L, V305I, P396L, L235V, F243L, R292P, Y300L, P396L, F243L, R292P, Y300L, V305I, P396L, K326W, E333S, S267E, H268E, S324T, S298A, E333A, K334A, L234Y, L235Q, G236W, S239M, H268D, D270E, S298A D270E, K326D, A330M, and/or K334E.

In some embodiments, the modified hIg Fc region comprises a hIgG1 Fc region comprising one or more amino acid variation relative to a reference hIgG1 Fc region.

In some embodiments, the hIgG1 Fc region comprises an amino acid substitution at one or more (e.g., 1, 2, or 3) of amino acid positions S298, E333, K334, EU numbering according to Kabat. In some embodiments, the hIgG1 Fc region comprises one or more (e.g., 1, 2, or 3) of the following amino acid substitutions S298A, E333A, and/or K334A, EU numbering according to Kabat.

In some embodiments, the hIgG1 Fc region comprises an amino acid substitution at one or more (e.g., 1 or 2) of amino acid positions S239 and/or I332, EU numbering according to Kabat. In some embodiments, the hIgG1 Fc region comprises one or more (e.g., 1 or 2) of the following amino acid substitutions S239D and/or I332E, EU numbering according to Kabat.

In some embodiments, the hIgG1 Fc region comprises an amino acid substitution at one or more (e.g., 1 or 2) of amino acid positions P247 and/or A339, EU numbering according to Kabat. In some embodiments, the hIgG1 Fc region comprises one or more (e.g., 1 or 2) of the following amino acid substitutions P247I and/or A339Q, EU numbering according to Kabat.

In some embodiments, the hIgG1 Fc region comprises an amino acid substitution at one or more (e.g., 1, 2, or 3) of amino acid positions S239, A330, and/or I332, EU numbering according to Kabat. In some embodiments, the hIgG1 Fc region comprises one or more (e.g., 1, 2, or 3) of the following amino acid substitutions S239D, A330L, and/or I332E, EU numbering according to Kabat.

In some embodiments, the hIgG1 Fc region comprises an amino acid substitution at one or more (e.g., 1, 2, or 3) of amino acid positions G236, S239, and/or I332, EU numbering according to Kabat. In some embodiments, the hIgG1 Fc region comprises one or more (e.g., 1, 2, or 3) of the following amino acid substitutions G236A, S239D, and/or I332E, EU numbering according to Kabat.

In some embodiments, the hIgG1 Fc region comprises an amino acid substitution at one or more (e.g., 1, 2, 3, 4, or 5) of amino acid positions F243, R292, Y300, V305, and/or P396, EU numbering according to Kabat. In some embodiments, the hIgG1 Fc region comprises one or more (e.g., 1, 2, 3, 4, or 5) of the following amino acid substitutions F243L, R292P, Y300L, V305I, and/or P396L, EU numbering according to Kabat.

In some embodiments, the hIgG1 Fc region comprises an amino acid substitution at one or more (e.g., 1, 2, 3, 4, or 5) of amino acid positions L235, F243, R292, Y300, and P396, EU numbering according to Kabat. In some embodiments, the hIgG1 Fc region comprises one or more (e.g., 1, 2, 3, 4, or 5) of the following amino acid substitutions L235V, F243L, R292P, Y300L, and/or P396L, EU numbering according to Kabat.

In some embodiments, the hIgG1 Fc region comprises an amino acid substitution at one or more (e.g., 1, 2, 3, 4, 5, 6, or 7) of amino acid positions L234, L235, G236, S239, H268, D270, and/or S298, EU numbering according to Kabat. In some embodiments, the hIgG1 Fc region comprises one or more (e.g., 1, 2, 3, 4, 5, 6, or 7) of the following amino acid substitutions L234Y, L235Q, G236W, S239M, H268D, D270E, and/or S298A, EU numbering according to Kabat.

In some embodiments, the hIgG1 Fc region comprises an amino acid substitution at one or more (e.g., 1, 2, 3, or 4) of amino acid positions D270, K326, A330, and/or K334, EU numbering according to Kabat. In some embodiments, the hIgG1 Fc region comprises one or more (e.g., 1, 2, 3, or 4) of the following amino acid substitutions D270E, K326D, A330M, and/or K334E, EU numbering according to Kabat.

In some embodiments, the hIgG1 Fc region comprises an amino acid substitution at one or more (e.g., 1, 2, 3, 4, or 5) of amino acid positions F243, R292, Y300, V305, and/or P396, EU numbering according to Kabat. In some embodiments, the hIgG1 Fc region comprises one or more (e.g., 1, 2, 3, 4, or 5) of the following amino acid substitutions F243L, R292P, Y300L, V305I, and/or P396L, EU numbering according to Kabat.

In some embodiments, the hIgG1 Fc region comprises an amino acid substitution at one or more (e.g., 1, 2, or 3) of amino acid positions S239, I332, and/or A330, EU numbering according to Kabat. In some embodiments, the hIgG1 Fc region comprises one or more (e.g., 1, 2, or 3) of the following amino acid substitutions S239D, I332E, and/or A330L, EU numbering according to Kabat.

In some embodiments, the hIgG1 Fc region comprises an amino acid substitution at one or more (e.g., 1, 2, 3, or 4) of amino acid positions S239, I332, A330, and/or G236, EU numbering according to Kabat. In some embodiments, the hIgG1 Fc region comprises one or more (e.g., 1, 2, 3, or 4) of the following amino acid substitutions S239D, I332E, A330L and/or G236A, EU numbering according to Kabat.

In some embodiments, the hIgG1 Fc region comprises an amino acid substitution at one or more (e.g., 1, 2, or 3) of amino acid positions S239, I332, and/or G326, EU numbering according to Kabat. In some embodiments, the hIgG1 Fc region comprises one or more (e.g., 1, 2, or 3) of the following amino acid substitutions S239D, I332E, and/or G326A, EU numbering according to Kabat.

In some embodiments, the hIgG1 Fc region comprises an amino acid substitution at amino acid position G326, EU numbering according to Kabat. In some embodiments, the hIgG1 Fc region comprises a G326A amino acid substitution, EU numbering according to Kabat.

In some embodiments, the hIgG1 Fc region comprises an amino acid substitution at one or more (e.g., 1, 2, or 3) of amino acid positions G236, S239, and/or I332, EU numbering according to Kabat. In some embodiments, the hIgG1 Fc region comprises one or more (e.g., 1, 2, or 3) of the following amino acid substitutions G236A, S239D, and/or I332E, EU numbering according to Kabat.

In some embodiments, the hIgG1 Fc region comprises an amino acid substitution at one or more (e.g., 1 or 2) of amino acid positions S239 and/or I332, EU numbering according to Kabat. In some embodiments, the hIgG1 Fc region comprises one or more (e.g., 1 or 2) of the following amino acid substitutions S239D and/or I332E, EU numbering according to Kabat.

In some embodiments, the hIgG1 Fc region comprises an amino acid substitution at one or more (e.g., 1 or 2) of amino acid positions K326 and/or E333, EU numbering according to Kabat. In some embodiments, the hIgG1 Fc region comprises one or more (e.g., 1 or 2) of the following amino acid substitutions K326W and/or E333S, EU numbering according to Kabat.

In some embodiments, the hIgG1 Fc region comprises an amino acid substitution at one or more (e.g., 1, 2, or 3) of amino acid positions S267, H268, and/or S324, EU numbering according to Kabat. In some embodiments, the hIgG1 Fc region comprises one or more (e.g., 1, 2, or 3) of the following amino acid substitutions S267E, H268E, and/or S324T, EU numbering according to Kabat.

In some embodiments, the hIgG1 Fc region comprises an amino acid substitution at one or more (e.g., 1, 2, or 3) of amino acid positions S298, E333, and/or K334, EU numbering according to Kabat. In some embodiments, the hIgG1 Fc region comprises one or more (e.g., 1, 2, or 3) of the following amino acid substitutions S298A, E333A, and/or K334A, EU numbering according to Kabat.

In some embodiments, the hIgG1 Fc region comprises an amino acid substitution at one or more (e.g., 1 or 2) of amino acid positions S239 and/or I332, EU numbering according to Kabat. In some embodiments, the hIgG1 Fc region comprises one or more (e.g., 1 or 2) of the following amino acid substitutions S239D and/or I332E, EU numbering according to Kabat.

In some embodiments, the hIgG1 Fc region comprises an amino acid substitution at one or more (e.g., 1 or 2) of amino acid positions P247 and/or A339, EU numbering according to Kabat. In some embodiments, the hIgG1 Fc region comprises one or more (e.g., 1 or 2) of the following amino acid substitutions P247I and/or A339Q, EU numbering according to Kabat.

In some embodiments, the hIg Fc region comprises one or more changes to the glycosylation. In some embodiments, the hIg Fc region is afucosylated. In some embodiments, the hIg Fc region is afucosylated and exhibits enhanced (e.g., increased) ADCC compared to a reference hIg Fc region that is not afucosylated.

### 5.5 Linkers

As described herein, the various components of a fusion protein described herein can each independently be directly operably connected via a peptide bond or indirectly operably connected via a linker.

In some embodiments, the fusion protein comprises one or more peptide linker. In some embodiments, the peptide linker is one or any combination of a cleavable linker, a non-cleavable linker, a flexible linker, a rigid linker, a helical linker, and/or a non-helical linker.

In some embodiments, the amino acid sequence of the peptide linker comprises from or from about 2-30, 5-30, 10-30, 15-30, 20-30, 25-30, 2-25, 5-25, 10-25, 15-25, 20-25, 2-20, 5-20, 10-20, 15-20, 2-15, 5-15, 10-15, 2-10, or 5-10 amino acid residues. In some embodiments, the amino acid sequence of the peptide linker comprises at least about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker comprises about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker consists of about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker comprises no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues. In some embodiments, the amino acid sequence of the peptide linker consists of no more than about 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues.

In some embodiments, the amino acid sequence of the peptide linker comprises glycine, serine, or both glycine and serine amino acid residues. In some embodiments, the amino acid sequence of the peptide linker comprises glycine, serine, and proline amino acid residues. In some embodiments, the amino acid sequence of the peptide linker consists of glycine, serine, or both glycine and serine amino acid residues. In some embodiments, the amino acid sequence of the peptide linker consists of glycine, serine, and proline amino acid residues.

The amino acid sequence of exemplary peptide linkers, which can be incorporated in one or more of the embodiments described herein (e.g., fusion proteins), is set provided in Table 14.

TABLE 14
The Amino Acid Sequence
of Exemplary Peptide Linkers.

| | | SEQ ID |
|---|---|---|
| Description | Amino Acid Sequence | NO |
| A | GGGGGGGS | 701 |
| B | GGGGGGGSGGGGGGGS | 702 |
| C | GGGGGGGSGGGGGGGSGGGGGGGS | 703 |
| D | GGGGS | 704 |
| E | GGGGSGGGGS | 705 |
| F | GGGGSGGGGSGGGGS | 706 |
| G | GGGS | 707 |
| H | GGGSGGGS | 708 |
| I | GGGSGGGSGGGS | 709 |
| J | GGGGGGGSGGGGSGGGGS | 710 |
| H | GGGGGGGGSGGGGSGGGGS | 711 |

In some embodiments, the amino acid sequence of the peptide linker comprises the amino acid sequence of any one of the linkers set forth in Table 14. In some embodiments, the amino acid sequence of the peptide linker consists of the amino acid sequence of any one of the linkers set forth in Table 14. In some embodiments, the amino acid sequence of the peptide linker comprises the amino acid sequence of any one of the linkers set forth in Table 14, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence of any one of the linkers set forth in Table 14, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence of any one of the linkers set forth in Table 14, comprising 1, 2, or 3 amino acid variations (e.g., substitutions, deletions, additions). In some embodiments, the amino acid sequence of the peptide linker consists of the amino acid sequence of any one of the linkers set forth in Table 14, comprising 1, 2, or 3 amino acid variations (e.g., substitutions, deletions, additions). In some embodiments, the amino acid sequence of the peptide linker comprises the amino acid sequence of any one of the linkers set forth in Table 14, and further comprises 1 or more but less than 15% (less than 12%, less than 10%, less than 8%), amino acid substitutions. In some embodiments, the amino acid sequence of the peptide linker consists of the amino acid sequence of any one of the linkers set forth in Table 14, and further comprises 1 or more but less than 15% (less than 12%, less than 10%, less than 8%), amino acid substitutions. In some embodiments, the amino acid sequence of the peptide linker comprises the amino acid sequence of any one of the linkers set forth in Table 14, comprising 1, 2, or 3 amino acid substitutions. In some embodiments, the amino acid sequence of the peptide linker consists of the amino acid sequence of any one of the linkers set forth in Table 14, comprising 1, 2, or 3 amino acid substitutions.

In some embodiments, the amino acid sequence of the peptide linker comprises the amino acid sequence set forth in any one of SEQ ID NOS: 701-711. In some embodiments, the amino acid sequence of the peptide linker consists of the amino acid sequence set forth in any one of SEQ ID NOS: 701-711. In some embodiments, the amino acid sequence of the peptide linker comprises the amino acid sequence set forth in any one of SEQ ID NOS: 701-711, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence set forth in any one of SEQ ID NOS: 701-711, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence set forth in any one of SEQ ID NOS: 701-711, comprising 1, 2, or 3 amino acid variations (e.g., substitutions, deletions, additions). In some embodiments, the amino acid sequence of the peptide linker consists of the amino acid sequence set forth in any one of SEQ ID NOS: 701-711, comprising 1, 2, or 3 amino acid variations (e.g., substitutions, deletions, additions). In some embodiments, the amino acid sequence of the peptide linker comprises the amino acid sequence set forth in any one of SEQ ID NOS: 701-711, and further comprises 1 or more but less than 15% (less than 12%, less than 10%, less than 8%), amino acid substitutions. In some embodiments, the amino acid sequence of the peptide linker consists of the amino acid sequence set forth in any one of SEQ ID NOS: 701-711, and further comprises 1 or more but less than 15% (less than 12%, less than 10%, less than 8%), amino acid substitutions. In some embodiments, the amino acid sequence of the peptide linker comprises the amino acid sequence set forth in any one of SEQ ID NOS: 701-711, comprising 1, 2, or 3 amino acid substitutions. In some embodiments, the amino acid sequence of the peptide linker consists of the amino acid sequence set forth in any one of SEQ ID NOS: 701-711, comprising 1, 2, or 3 amino acid substitutions.

In some embodiments, the amino acid sequence of the peptide linker comprises the amino acid sequence set forth in SEQ ID NO: 710. In some embodiments, the amino acid sequence of the peptide linker consists of the amino acid sequence set forth in SEQ ID NO: 710. In some embodiments, the amino acid sequence of the peptide linker comprises the amino acid sequence set forth in SEQ ID NO: 710, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence set forth in SEQ ID NO: 710, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence set forth in SEQ ID NO: 710, comprising 1, 2, or 3 amino acid variations (e.g., substitutions, deletions, additions). In some embodiments, the amino acid sequence of the peptide linker consists of the amino acid sequence set forth in SEQ ID NO: 710, comprising 1, 2, or 3 amino acid variations (e.g., substitutions, deletions, additions). In some embodiments, the amino acid sequence of the peptide linker comprises the amino acid sequence set forth in SEQ ID NO: 710, and further comprises 1 or more but less than 15% (less than 12%, less than 10%, less than 8%), amino acid substitutions. In some embodiments, the amino acid sequence of the peptide linker consists of the amino acid sequence set forth in SEQ ID NO: 710, and further comprises 1 or more but less than 15% (less than 12%, less than 10%, less than 8%), amino acid substitutions. In some embodiments, the amino acid sequence of the peptide linker comprises the amino acid sequence set forth in SEQ ID NO: 710, comprising 1, 2, or 3 amino acid substitutions. In some embodiments, the amino acid sequence of the peptide linker consists of the amino acid sequence set forth in SEQ ID NO: 710, comprising 1, 2, or 3 amino acid substitutions.

In some embodiments, the amino acid sequence of the peptide linker comprises the amino acid sequence set forth in SEQ ID NO: 704. In some embodiments, the amino acid sequence of the peptide linker consists of the amino acid sequence set forth in SEQ ID NO: 704. In some embodiments, the amino acid sequence of the peptide linker comprises the amino acid sequence set forth in SEQ ID NO: 704, and further comprises 1 or more but less than 15% (less than 12%, less than 10%, less than 8%), amino acid variations (e.g., amino acid substitutions, deletions, or additions). In some embodiments, the amino acid sequence of the peptide linker consists of the amino acid sequence set forth in SEQ ID NO: 704, and further comprises 1 or more but less than 15% (less than 12%, less than 10%, less than 8%), amino acid variations (e.g., amino acid substitutions, deletions, or additions). In some embodiments, the amino acid sequence of the peptide linker comprises the amino acid sequence set forth in SEQ ID NO: 704, comprising 1, 2, or 3 amino acid variations (e.g., substitutions, deletions, additions). In some embodiments, the amino acid sequence of the peptide linker consists of the amino acid sequence set forth in SEQ ID NO: 704, comprising 1, 2, or 3 amino acid variations (e.g., substitutions, deletions, additions). In some embodiments, the amino acid sequence of the peptide linker comprises the amino acid sequence set forth in SEQ ID NO: 704, and further comprises 1 or more but less than 15% (less than 12%, less than 10%, less than 8%), amino acid substitutions. In some embodiments, the amino acid sequence of the peptide linker consists of the amino acid sequence set forth in SEQ ID NO: 704, and further comprises 1 or more but less than 15% (less than 12%, less than 10%, less than 8%), amino acid substitutions. In some embodiments, the amino acid sequence of the peptide linker comprises the amino acid sequence set forth in SEQ ID NO: 704, comprising 1, 2, or 3 amino acid substitutions. In some embodiments, the amino acid sequence of the peptide linker consists of the amino acid sequence set forth in SEQ ID NO: 704, comprising 1, 2, or 3 amino acid substitutions.

### 5.6 Signal Peptides

In some embodiments, a fusion protein described herein (or one or more polypeptide thereof) comprises one or more signal peptide. In some embodiments, the fusion protein (or one or more polypeptide thereof) comprises a homologous signal peptide. In some embodiments, the fusion protein (or one or more polypeptide thereof) comprises a heterologous signal peptide. In some embodiments, the protein (or one or more polypeptide thereof) comprises a heterologous signal peptide operably connected to the N-terminus of the protein.

Commonly used heterologous signal peptides are known in the art, for example, the native signal peptide of human interleukin 2 (hIL-2), human oncostatin M (hOSM), human chymotrypsinogen (hCTRB1), human trypsinogen 2 (hTRY2), and human insulin (hINS). A person of ordinary skill can determine the appropriate signal peptide using standard methodology known in the art. The amino acid sequence of exemplary signal peptides is provided in Table 15.

TABLE 15
The Amino Acid Sequence
of Exemplary Signal Peptides.

| | | SEQ ID |
|---|---|---|
| Description | Amino Acid Sequence | NO |
| hIL-2 | MYRMQLLSCIALSLALVTNS | 712 |
| hOSM | MGVLLTQRTLLSLVLALLFPSMASM | 713 |
| hCTRB1 | MASLWLLSCFSLVGAAFG | 714 |
| hTRY2 | MNLLLILTFVAAAVA | 715 |
| hINS | MALWMRLLPLLALLALWGPDPAAA | 716 |

In some embodiments, the amino acid sequence of the signal peptide comprises the amino acid sequence of any one of the signal peptides set forth in Table 15. In some embodiments, the amino acid sequence of the signal peptide comprises the amino acid sequence of any one of the signal peptides set forth in Table 15, and further comprises 1 or more but less than 15% (less than 12%, less than 10%, less than 8%), amino acid variations (e.g., amino acid substitutions, deletions, or additions). In some embodiments, the amino acid sequence of the signal peptide comprises the amino acid sequence of any one of the signal peptides set forth in Table 15, comprising 1, 2, or 3 amino acid variations (e.g., substitutions, deletions, additions). In some embodiments, the amino acid sequence of the signal peptide comprises the amino acid sequence of any one of the signal peptides set forth in Table 15, and further comprises 1 or more but less than 15% (less than 12%, less than 10%, less than 8%), amino acid substitutions. In some embodiments, the amino acid sequence of the signal peptide comprises the amino acid sequence of any one of the signal peptides set forth in Table 15, comprising 1, 2, or 3 amino acid substitutions.

In some embodiments, the amino acid sequence of the signal peptide consists of the amino acid sequence of any one of the signal peptides set forth in Table 15. In some embodiments, the amino acid sequence of the signal peptide consists of the amino acid sequence of any one of the signal peptides set forth in Table 15, and further consists of 1 or more but less than 15% (less than 12%, less than 10%, less than 8%), amino acid variations (e.g., amino acid substitutions, deletions, or additions). In some embodiments, the amino acid sequence of the signal peptide consists of the amino acid sequence of any one of the signal peptides set forth in Table 15, comprising 1, 2, or 3 amino acid variations (e.g., substitutions, deletions, additions). In some embodiments, the amino acid sequence of the signal peptide consists of the amino acid sequence of any one of the signal peptides set forth in Table 15, and further consists of 1 or more but less than 15% (less than 12%, less than 10%, less than 8%), amino acid substitutions. In some embodiments, the amino acid sequence of the signal peptide consists of the amino acid sequence of any one of the signal peptides set forth in Table 15, comprising 1, 2, or 3 amino acid substitutions.

In some embodiments, the amino acid sequence of the signal peptide comprises the amino acid sequence set forth in any one of SEQ ID NOS: 712-716. In some embodiments, the amino acid sequence of the signal peptide comprises the amino acid sequence set forth in any one of SEQ ID NOS: 712-716, and further comprises 1 or more but less than 15% (less than 12%, less than 10%, less than 8%), amino acid variations (e.g., amino acid substitutions, deletions, or additions). In some embodiments, the amino acid sequence of the signal peptide comprises the amino acid sequence set forth in any one of SEQ ID NOS: 712-716, comprising 1, 2, or 3 amino acid variations (e.g., substitutions, deletions, additions). In some embodiments, the amino acid sequence of the signal peptide comprises the amino acid sequence set forth in any one of SEQ ID NOS: 712-716, and further comprises 1 or more but less than 15% (less than 12%, less than 10%, less than 8%), amino acid substitutions. In some embodiments, the amino acid sequence of the signal peptide comprises the amino acid sequence set forth in any one of SEQ ID NOS: 712-716, comprising 1, 2, or 3 amino acid substitutions.

In some embodiments, the amino acid sequence of the signal peptide consists of the amino acid sequence set forth in any one of SEQ ID NOS: 712-716. In some embodiments, the amino acid sequence of the signal peptide consists of the amino acid sequence set forth in any one of SEQ ID NOS: 712-716, and further consists of 1 or more but less than 15% (less than 12%, less than 10%, less than 8%), amino acid variations (e.g., amino acid substitutions, deletions, or additions). In some embodiments, the amino acid sequence of the signal peptide consists of the amino acid sequence set forth in any one of SEQ ID NOS: 712-716, comprising 1, 2, or 3 amino acid variations (e.g., substitutions, deletions, additions). In some embodiments, the amino acid sequence of the signal peptide consists of the amino acid sequence set forth in any one of SEQ ID NOS: 712-716, and further consists of 1 or more but less than 15% (less than 12%, less than 10%, less than 8%), amino acid substitutions. In some embodiments, the amino acid sequence of the signal peptide consists of the amino acid sequence set forth in any one of SEQ ID NOS: 712-716, comprising 1, 2, or 3 amino acid substitutions.

### 5.7 Components & Formats

The fusion proteins described herein can comprise a variety of components, including one or more (e.g., at least two) immunomodulatory proteins described herein (see, e.g., §§ 5.2.1.1, 5.2.1.2, 5.2.1.3, and 5.2.1.4); and in some embodiments, one or more additional moiety (e.g., heterologous moiety) (e.g., described herein (e.g., an antibody, Ig constant region, an Fc region, etc.) (see, e.g., §§ 5.3 and 5.4). Exemplary combinations of components and formats are provided below.

### 5.7.1 Exemplary Combinations of Immunomodulatory Protein Components

The fusion proteins described herein can comprise various immunomodulatory proteins described herein, see, e.g., §§ 5.2.1.1, 5.2.1.2, 5.2.1.3, and 5.2.1.4, and combinations thereof.

In some embodiments, the fusion protein is monospecific. In some embodiments, the fusion protein is bispecific. In some embodiments, the fusion protein is trispecific. In some embodiments, the fusion protein is bivalent. In some embodiments, the fusion protein is trivalent. In some embodiments, the fusion protein is monospecific and bivalent. In some embodiments, the fusion protein is bispecific and bivalent. In some embodiments, the fusion protein is trispecific and trivalent. In some embodiments, the fusion protein is bispecific and trivalent.

For example, in some embodiments, the fusion protein comprises a plurality of immunomodulatory proteins described herein. In some embodiments, the fusion protein comprises at least 2, 3, 4, 5, or 6, or more immunomodulatory proteins described herein. In some embodiments the fusion protein comprises 2, 3, 4, 5, or 6, or more immunomodulatory proteins described herein. In some embodiments the fusion protein comprises 2 immunomodulatory proteins described herein. In some embodiments the fusion protein comprises 3 immunomodulatory proteins described herein. In some embodiments the fusion protein comprises at least 2 but no more than 3, 4, 5, or 6, or more immunomodulatory proteins described herein.

In some embodiments, the fusion protein comprises a plurality of different immunomodulatory proteins described herein. In some embodiments the fusion protein comprises at least 2, 3, 4, 5, or 6, or more different immunomodulatory proteins described herein. In some embodiments the fusion protein comprises 2, 3, 4, 5, or 6, or more different immunomodulatory proteins described herein. In some embodiments the fusion protein comprises 2 different immunomodulatory proteins described herein. In some embodiments the fusion protein comprises 3 different immunomodulatory proteins described herein. In some embodiments the fusion protein comprises at least 2 but no more than 3, 4, 5, or 6, or more different immunomodulatory proteins described herein.

In some embodiments, the fusion protein comprises a plurality of immunomodulatory proteins described herein, wherein each immunomodulatory protein of the fusion protein specifically binds to a different target (e.g., hIL-10R, TL1A, TNFα, CD30L). In some embodiments the fusion protein comprises at least 2, 3, 4, 5, or 6, or more immunomodulatory proteins described herein, wherein each immunomodulatory protein of the fusion protein specifically binds to a different target (e.g., hIL-10R, TL1A, TNFα, CD30L). In some embodiments the fusion protein comprises 2, 3, 4, 5, or 6, or more immunomodulatory proteins described herein, wherein each immunomodulatory protein of the fusion protein specifically binds to a different target (e.g., hIL-10R, TL1A, TNFα, CD30L). In some embodiments the fusion protein comprises 2 immunomodulatory proteins described herein, wherein each immunomodulatory protein of the fusion protein specifically binds to a different target (e.g., hIL-10R, TL1A, TNFα, CD30L). In some embodiments the fusion protein comprises 3 immunomodulatory proteins described herein, wherein each immunomodulatory protein of the fusion protein specifically binds to a different target (e.g., hIL-10R, TL1A, TNFα, CD30L). In some embodiments the fusion protein comprises at least 2 but no more than 3, 4, 5, or 6, or more immunomodulatory proteins described herein, wherein each immunomodulatory protein of the fusion protein specifically binds to a different target (e.g., hIL-10R, TL1A, TNFα, CD30L).

In some embodiments, the fusion protein comprises a plurality of the same immunomodulatory proteins described herein. In some embodiments the fusion protein comprises at least 2, 3, 4, 5, or 6, or more of the same immunomodulatory proteins described herein. In some embodiments the fusion protein comprises 2, 3, 4, 5, or 6, or more of the same immunomodulatory proteins described herein. In some embodiments the fusion protein comprises 2 of the same immunomodulatory proteins described herein. In some embodiments the fusion protein comprises 3 of the same immunomodulatory proteins described herein. In some embodiments the fusion protein comprises at least 2 but no more than 3, 4, 5, or 6, or more of the same immunomodulatory proteins described herein.

In some embodiments, the fusion protein comprises a plurality of immunomodulatory proteins described herein, wherein the amino acid sequence of each member of the plurality is 100% identical. In some embodiments the fusion protein comprises at least 2, 3, 4, 5, or 6, or more of immunomodulatory proteins described herein, wherein the amino acid sequence of each of the at least 2, 3, 4, 5, or 6, or more of immunomodulatory proteins is 100% identical. In some embodiments, the fusion protein comprises 2, 3, 4, 5, or 6, or more of immunomodulatory proteins described herein, wherein the amino acid sequence of 2, 3, 4, 5, or 6, or more of immunomodulatory proteins is 100% identical. In some embodiments the fusion protein comprises 2 of immunomodulatory proteins described herein, wherein the amino acid sequence of each of the 2 immunomodulatory proteins is 100% identical. In some embodiments the fusion protein comprises 3 of immunomodulatory proteins described herein, wherein the amino acid sequence of each of the 3 immunomodulatory proteins is 100% identical. In some embodiments the fusion protein comprises at least 2 but no more than 3, 4, 5, or 6, or more of immunomodulatory proteins described herein, wherein the amino acid sequence of each of the at least 2 immunomodulatory proteins are 100% identical.

In some embodiments, the fusion protein comprises a plurality of immunomodulatory proteins described herein, wherein each immunomodulatory protein of the fusion protein specifically binds to the same target (e.g., hIL-10R, TL1A, TNFα, CD30L). In some embodiments the fusion protein comprises at least 2, 3, 4, 5, or 6, or more immunomodulatory proteins described herein, wherein each immunomodulatory protein of the fusion protein specifically binds to the same target (e.g., hIL-10R, TL1A, TNFα, CD30L). In some embodiments the fusion protein comprises 2, 3, 4, 5, or 6, or more immunomodulatory proteins described herein, wherein each immunomodulatory protein of the fusion protein specifically binds to the same target (e.g., hIL-10R, TL1A, TNFα, CD30L). In some embodiments the fusion protein comprises 2 immunomodulatory proteins described herein, wherein each immunomodulatory protein of the fusion protein specifically binds to the same target (e.g., hIL-10R, TL1A, TNFα, CD30L). In some embodiments the fusion protein comprises 3 immunomodulatory proteins described herein, wherein each immunomodulatory protein of the fusion protein specifically binds to the same target (e.g., hIL-10R, TL1A, TNFα, CD30L). In some embodiments the fusion protein comprises at least 2 but no more than 3, 4, 5, or 6, or more immunomodulatory proteins described herein, wherein each immunomodulatory protein of the fusion protein specifically binds to the same target (e.g., hIL-10R, TL1A, TNFα, CD30L).

In some embodiments, the fusion protein comprises (a) a protein that specifically binds hIL-10R (e.g., described herein, see, e.g., Table 2); and any one or more of (b) a protein that specifically binds hTL1A (e.g., described herein, see, e.g., Table 3); (c) a protein that specifically binds TNFα (e.g., described herein, see, e.g., Table 4); (d) a protein that specifically binds CD30L (e.g., described herein, see, e.g., Table 5); and/or (e) a protein that specifically binds hIL-10R (e.g., described herein, see, e.g., Table 2).

In some embodiments, the fusion protein comprises (a) a protein that specifically binds hIL-10R (e.g., described herein, see, e.g., Table 2); and (e) a protein that specifically binds hIL-10R (e.g., described herein, see, e.g., Table 2). In some embodiments, the amino acid sequence of protein (a) and protein (e) is 100% identical. In some embodiments, the amino acid sequence of protein (a) and protein (e) is not 100% identical. In some embodiments, protein (a) and protein (e) are the same. In some embodiments, protein (a) and protein (e) are different.

In some embodiments, the fusion protein comprises (b) a protein that specifically binds hTL1A (e.g., described herein, see, e.g., Table 3); and any one or more of ((a) a protein that specifically binds hIL-10R (e.g., described herein, see, e.g., Table 2); (c) a protein that specifically binds TNFα (e.g., described herein, see, e.g., Table 4); (d) a protein that specifically binds CD30L (e.g., described herein, see, e.g., Table 5); and/or (e) a protein that specifically binds hTL1A (e.g., described herein, see, e.g., Table 3).

In some embodiments, the fusion protein comprises (b) a protein that specifically binds hTL1A (e.g., described herein, see, e.g., Table 3); and (e) a protein that specifically binds hTL1A (e.g., described herein, see, e.g., Table 3). In some embodiments, the amino acid sequence of protein (b) and protein (e) is 100% identical. In some embodiments, the amino acid sequence of protein (b) and protein (e) is not 100% identical. In some embodiments, protein (b) and protein (e) are the same. In some embodiments, protein (b) and protein (e) are different.

In some embodiments, the fusion protein comprises (c) a protein that specifically binds TNFα (e.g., described herein, see, e.g., Table 4); and any one or more of (a) a protein that specifically binds hIL-10R (e.g., described herein, see, e.g., Table 2); (b) a protein that specifically binds hTL1A (e.g., described herein, see, e.g., Table 3); (d) a protein that specifically binds CD30L (e.g., described herein, see, e.g., Table 5); and/or (e) a protein that specifically binds TNFα (e.g., described herein, see, e.g., Table 4).

In some embodiments, the fusion protein comprises (c) a protein that specifically binds TNFα (e.g., described herein, see, e.g., Table 4); and (e) a protein that specifically binds TNFα (e.g., described herein, see, e.g., Table 4). In some embodiments, the amino acid sequence of protein (c) and protein (e) is 100% identical. In some embodiments, the amino acid sequence of protein (c) and protein (e) is not 100% identical. In some embodiments, protein (c) and protein (e) are the same. In some embodiments, protein (c) and protein (e) are different.

In some embodiments, the fusion protein comprises (d) a protein that specifically binds CD30L (e.g., described herein, see, e.g., Table 5); and any one or more of (b) a protein that specifically binds hTL1A (e.g., described herein, see, e.g., Table 3); (c) a protein that specifically binds TNFα (e.g., described herein, see, e.g., Table 4); (a) a protein that specifically binds hIL-10R (e.g., described herein, see, e.g., Table 2); and/or (e) a protein that specifically binds CD30L (e.g., described herein, see, e.g., Table 5).

In some embodiments, the fusion protein comprises (d) a protein that specifically binds CD30L (e.g., described herein, see, e.g., Table 5); and (e) a protein that specifically binds CD30L (e.g., described herein, see, e.g., Table 5). In some embodiments, the amino acid sequence of protein (d) and protein (e) is 100% identical. In some embodiments, the amino acid sequence of protein (d) and protein (e) is not 100% identical. In some embodiments, protein (d) and protein (e) are the same. In some embodiments, protein (d) and protein (e) are different.

In some embodiments, the fusion protein comprises (a) a protein that specifically binds hIL-10R (e.g., described herein, see, e.g., Table 2); and any one or more of (b) a protein that specifically binds hTL1A (e.g., described herein, see, e.g., Table 3); (c) a protein that specifically binds TNFα (e.g., described herein, see, e.g., Table 4); and/or (d) a protein that specifically binds CD30L (e.g., described herein, see, e.g., Table 5).

In some embodiments, the fusion protein comprises (b) a protein that specifically binds hTL1A (e.g., described herein, see, e.g., Table 3); and any one or more of (a) a protein that specifically binds hIL-10R (e.g., described herein, see, e.g., Table 2); (c) a protein that specifically binds TNFα (e.g., described herein, see, e.g., Table 4); and/or (d) a protein that specifically binds CD30L (e.g., described herein, see, e.g., Table 5).

In some embodiments, the fusion protein comprises (c) a protein that specifically binds TNFα (e.g., described herein, see, e.g., Table 4); and any one or more of (b) a protein that specifically binds hTL1A (e.g., described herein, see, e.g., Table 3); (a) a protein that specifically binds hIL-10R (e.g., described herein, see, e.g., Table 2); and/or (d) a protein that specifically binds CD30L (e.g., described herein, see, e.g., Table 5).

In some embodiments, the fusion protein comprises (d) a protein that specifically binds CD30L (e.g., described herein, see, e.g., Table 5); and any one or more of (b) a protein that specifically binds hTL1A (e.g., described herein, see, e.g., Table 3); (c) a protein that specifically binds TNFα (e.g., described herein, see, e.g., Table 4); and/or (a) a protein that specifically binds hIL-10R (e.g., described herein, see, e.g., Table 2).

In some embodiments, the fusion protein comprises (a) protein that specifically binds hIL-10R (e.g., described herein, see, e.g., Table 2); and (b) a protein that specifically binds hTL1A (e.g., described herein, see, e.g., Table 3).

In some embodiments, the fusion protein comprises (a) a protein that specifically binds hIL-10R (e.g., described herein, see, e.g., Table 2); and (c) a protein that specifically binds TNFα (e.g., described herein, see, e.g., Table 4).

In some embodiments, the fusion protein comprises (a) a protein that specifically binds hIL-10R (e.g., described herein, see, e.g., Table 2); and (d) a protein that specifically binds CD30L (e.g., described herein, see, e.g., Table 5).

In some embodiments, the fusion protein comprises (b) a protein that specifically binds hTL1A (e.g., described herein, see, e.g., Table 3) and (c) a protein that specifically binds TNFα (e.g., described herein, see, e.g., Table 4).

In some embodiments, the fusion protein comprises (b) a protein that specifically binds hTL1A (e.g., described herein, see, e.g., Table 3) and (d) a protein that specifically binds CD30L (e.g., described herein, see, e.g., Table 5).

In some embodiments, the fusion protein comprises (c) a protein that specifically binds TNFα (e.g., described herein, see, e.g., Table 4) and (d) a protein that specifically binds CD30L (e.g., described herein, see, e.g., Table 5).

In some embodiments, the fusion protein comprises (a) a protein that specifically binds hIL-10R (e.g., described herein, see, e.g., Table 2); (b) a protein that specifically binds hTL1A (e.g., described herein, see, e.g., Table 3); and (c) a protein that specifically binds TNFα (e.g., described herein, see, e.g., Table 4).

In some embodiments, the fusion protein comprises (a) a protein that specifically binds hIL-10R (e.g., described herein, see, e.g., Table 2); (b) a protein that specifically binds hTL1A (e.g., described herein, see, e.g., Table 3); and (d) a protein that specifically binds CD30L (e.g., described herein, see, e.g., Table 5).

In some embodiments, the fusion protein comprises (a) a protein that specifically binds hIL-10R (e.g., described herein, see, e.g., Table 2); (c) a protein that specifically binds TNFα (e.g., described herein, see, e.g., Table 4); and (d) a protein that specifically binds CD30L (e.g., described herein, see, e.g., Table 5).

In some embodiments, the fusion protein comprises (b) a protein that specifically binds hTL1A (e.g., described herein, see, e.g., Table 3); (c) a protein that specifically binds TNFα (e.g., described herein, see, e.g., Table 4); and (d) a protein that specifically binds CD30L (e.g., described herein, see, e.g., Table 5).

In some embodiments, the fusion protein comprises (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and any one or more of (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576; and/or (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386.

In some embodiments, the fusion protein comprises (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386. In some embodiments, the amino acid sequence of protein (a) and protein (e) is 100% identical. In some embodiments, the amino acid sequence of protein (a) and protein (e) is not 100% identical. In some embodiments, protein (a) and protein (e) are the same. In some embodiments, protein (a) and protein (e) are different.

In some embodiments, the fusion protein comprises (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; and any one or more of (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576; and/or (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454.

In some embodiments, the fusion protein comprises (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; and (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454. In some embodiments, the amino acid sequence of protein (b) and protein (e) is 100% identical. In some embodiments, the amino acid sequence of protein (b) and protein (e) is not 100% identical. In some embodiments, protein (b) and protein (e) are the same. In some embodiments, protein (b) and protein (e) are different.

In some embodiments, the fusion protein comprises (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and any one or more of (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576; and/or (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464.

In some embodiments, the fusion protein comprises (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464. In some embodiments, the amino acid sequence of protein (c) and protein (e) is 100% identical. In some embodiments, the amino acid sequence of protein (c) and protein (e) is not 100% identical. In some embodiments, protein (c) and protein (e) are the same. In some embodiments, protein (c) and protein (e) are different.

In some embodiments, the fusion protein comprises (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576; and any one or more of (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and/or (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576; and (e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576. In some embodiments, the amino acid sequence of protein (d) and protein (e) is 100% identical. In some embodiments, the amino acid sequence of protein (d) and protein (e) is not 100% identical. In some embodiments, protein (d) and protein (e) are the same. In some embodiments, protein (d) and protein (e) are different.

In some embodiments, the fusion protein comprises (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and any one or more of (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and/or (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; and any one or more of; (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386 (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and/or (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and any one or more of (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and/or (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576; and any one or more of (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and/or (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386.

In some embodiments, the fusion protein comprises (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454.

In some embodiments, the fusion protein comprises (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464.

In some embodiments, the fusion protein comprises (a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454 and (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464.

In some embodiments, the fusion protein comprises (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454 and (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464 and (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; and (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464.

In some embodiments, the fusion protein comprises a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; and (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; operably connected to any one or more of (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454; (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and (d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

In some embodiments, the fusion protein comprises (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in NO: 382; and (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in SEQ ID NO: 455. In some embodiments, the fusion protein comprises (b) a protein comprising an amino acid sequence at least about 90%, 91%, 92%, 93%, 94%, 95%, 96%, 97%, 98%, 99%, or 100% identical to the amino acid sequence set forth in NO: 382; and (c) a protein comprising an amino acid sequence at least 90%, 91%, 92%, 93%, 94%, 95%, 96%, 97%, 98%, 99%, or 100% identical to the amino acid sequence set forth in SEQ ID NO: 455. In some embodiments, the fusion protein comprises (b) a protein comprising an amino acid sequence at least about 95%, 96%, 97%, 98%, 99%, or 100% identical to the amino acid sequence set forth in NO: 382; and (c) a protein comprising an amino acid sequence at least about 95%, 96%, 97%, 98%, 99%, or 100% identical to the amino acid sequence set forth in SEQ ID NO: 455. In some embodiments, the fusion protein comprises (b) a protein comprising the amino acid sequence set forth in NO: 382; and (c) a protein comprising the amino acid sequence set forth in SEQ ID NO: 455.

In some embodiments, the fusion protein comprises (b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in NO: 413; and (c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in SEQ ID NO: 455. In some embodiments, the fusion protein comprises (b) a protein comprising an amino acid sequence at least about 90%, 91%, 92%, 93%, 94%, 95%, 96%, 97%, 98%, 99%, or 100% identical to the amino acid sequence set forth in NO: 413; and (c) a protein comprising an amino acid sequence at least 90%, 91%, 92%, 93%, 94%, 95%, 96%, 97%, 98%, 99%, or 100% identical to the amino acid sequence set forth in SEQ ID NO: 455. In some embodiments, the fusion protein comprises (b) a protein comprising an amino acid sequence at least about 95%, 96%, 97%, 98%, 99%, or 100% identical to the amino acid sequence set forth in NO: 413; and (c) a protein comprising an amino acid sequence at least about 95%, 96%, 97%, 98%, 99%, or 100% identical to the amino acid sequence set forth in SEQ ID NO: 455. In some embodiments, the fusion protein comprises (b) a protein comprising the amino acid sequence set forth in NO: 413; and (c) a protein comprising the amino acid sequence set forth in SEQ ID NO: 455.

### 5.7.2 Exemplary Formats

The various components of a fusion protein described herein can be arranged in any configuration or order as long as each component maintains the ability to mediate its function (e.g., bind to its cognate partner). Exemplary formats are provided below. The formats are intended to exemplary only and not intended to be limiting in any way.

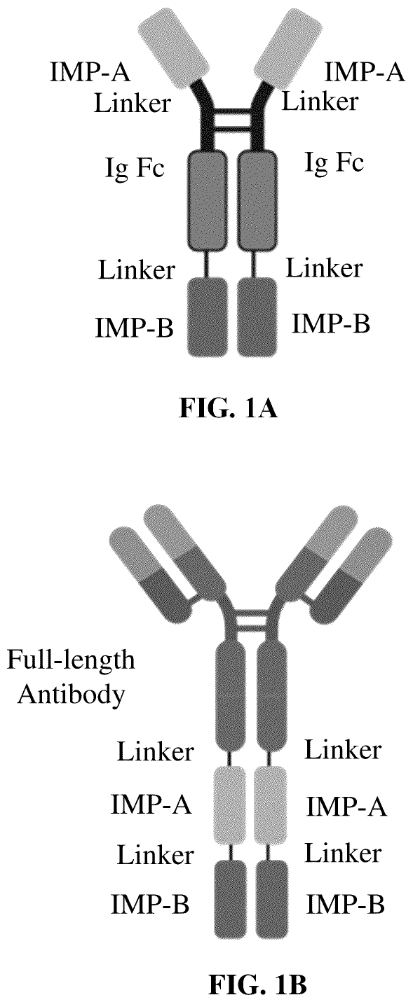

In some embodiments, the C-terminus of a first immunomodulatory protein described herein (see, e.g., §§ 5.2.1.1, 5.2.1.2, 5.2.1.3, and 5.2.1.4) is operably connected (either directly or indirectly through a peptide linker) to the N-terminus of an Ig (e.g., hIg) Fc region, and the N-terminus of a second immunomodulatory protein described herein (see, e.g., §§ 5.2.1.1, 5.2.1.2, 5.2.1.3, and 5.2.1.4) is operably connected (either directly or indirectly through a peptide linker) to the C-terminus of the Ig (e.g., hIg) Fc region. See, e.g., FIG. 1A. In embodiments, the first immunomodulatory protein and the second immunomodulatory protein specifically bind different targets. For example, in some embodiments, the first immunomodulatory protein specifically binds the hIL10-R (e.g., proteins described in Table 2); and the second immunomodulatory protein specifically binds TL1A (e.g., proteins described in Table 3). The combinations can be, for example, any described above in § 5.7.1.

In some embodiments, the C-terminus of a first immunomodulatory protein described herein (see, e.g., §§ 5.2.1.1, 5.2.1.2, 5.2.1.3, and 5.2.1.4) is operably connected indirectly through a peptide linker to the N-terminus of an Ig (e.g., hIg) Fc region, and the N-terminus of a second immunomodulatory protein described herein (see, e.g., §§ 5.2.1.1, 5.2.1.2, 5.2.1.3, and 5.2.1.4) is operably connected indirectly through a peptide linker to the C-terminus of the Ig (e.g., hIg) Fc region. See, e.g., FIG. 1A. In embodiments, the first immunomodulatory protein and the second immunomodulatory protein specifically bind different targets. For example, in some embodiments, the first immunomodulatory protein specifically binds the hIL10-R (e.g., proteins described in Table 2); and the second immunomodulatory protein specifically binds TL1A (e.g., proteins described in Table 3). The combinations can be, for example, any described above in § 5.7.1.

In some embodiments, the N-terminus of a first immunomodulatory protein described herein (see, e.g., §§ 5.2.1.1, 5.2.1.2, 5.2.1.3, and 5.2.1.4) is operably connected (either directly or indirectly through a peptide linker) to the C-terminus an Ig (e.g., hIg) Fc region, and the N-terminus of a second immunomodulatory protein described herein (see, e.g., §§ 5.2.1.1, 5.2.1.2, 5.2.1.3, and 5.2.1.4) is operably connected (either directly or indirectly through a peptide linker) to the C-terminus of the first immunomodulatory protein. In embodiments, the first immunomodulatory protein and the second immunomodulatory protein specifically bind different targets. For example, in some embodiments, the first immunomodulatory protein specifically binds the hIL10-R (e.g., proteins described in Table 2); and the second immunomodulatory protein specifically binds TL1A (e.g., proteins described in Table 3). The combinations can be, for example, any described above in § 5.7.1.

In some embodiments, the N-terminus of a first immunomodulatory protein described herein (see, e.g., §§ 5.2.1.1, 5.2.1.2, 5.2.1.3, and 5.2.1.4) is operably connected indirectly through a peptide linker to the C-terminus an Ig (e.g., hIg) Fc region, and the N-terminus of a second immunomodulatory protein described herein (see, e.g., §§ 5.2.1.1, 5.2.1.2, 5.2.1.3, and 5.2.1.4) is operably connected indirectly through a peptide linker to the C-terminus of the first immunomodulatory protein. In embodiments, the first immunomodulatory protein and the second immunomodulatory protein specifically bind different targets. For example, in some embodiments, the first immunomodulatory protein specifically binds the hIL10-R (e.g., proteins described in Table 2); and the second immunomodulatory protein specifically binds TL1A (e.g., proteins described in Table 3). The combinations can be, for example, any described above in § 5.7.1.

In some embodiments, the N-terminus of a first immunomodulatory protein described herein (see, e.g., §§ 5.2.1.1, 5.2.1.2, 5.2.1.3, and 5.2.1.4) is operably connected (either directly or indirectly through a peptide linker) to the C-terminus a full-length antibody, and the N-terminus of a second immunomodulatory protein described herein (see, e.g., §§ 5.2.1.1, 5.2.1.2, 5.2.1.3, and 5.2.1.4) is operably connected (either directly or indirectly through a peptide linker) to the C-terminus of the first immunomodulatory protein. In embodiments, the first immunomodulatory protein and the second immunomodulatory protein specifically bind different targets. For example, in some embodiments, the first immunomodulatory protein specifically binds the hIL10-R (e.g., proteins described in Table 2); and the second immunomodulatory protein specifically binds TL1A (e.g., proteins described in Table 3). See, e.g., FIG. 1B. The combinations can be, for example, any described above in § 5.7.1.

In some embodiments, the N-terminus of a first immunomodulatory protein described herein (see, e.g., §§ 5.2.1.1, 5.2.1.2, 5.2.1.3, and 5.2.1.4) is operably connected indirectly through a peptide linker to the C-terminus a full-length antibody, and the N-terminus of a second immunomodulatory protein described herein (see, e.g., §§ 5.2.1.1, 5.2.1.2, 5.2.1.3, and 5.2.1.4) is operably connected indirectly through a peptide linker to the C-terminus of the first immunomodulatory protein. In embodiments, the first immunomodulatory protein and the second immunomodulatory protein specifically bind different targets. For example, in some embodiments, the first immunomodulatory protein specifically binds the hIL10-R (e.g., proteins described in Table 2); and the second immunomodulatory protein specifically binds TL1A (e.g., proteins described in Table 3). See, e.g., FIG. 1B. The combinations can be, for example, any described above in § 5.7.1.

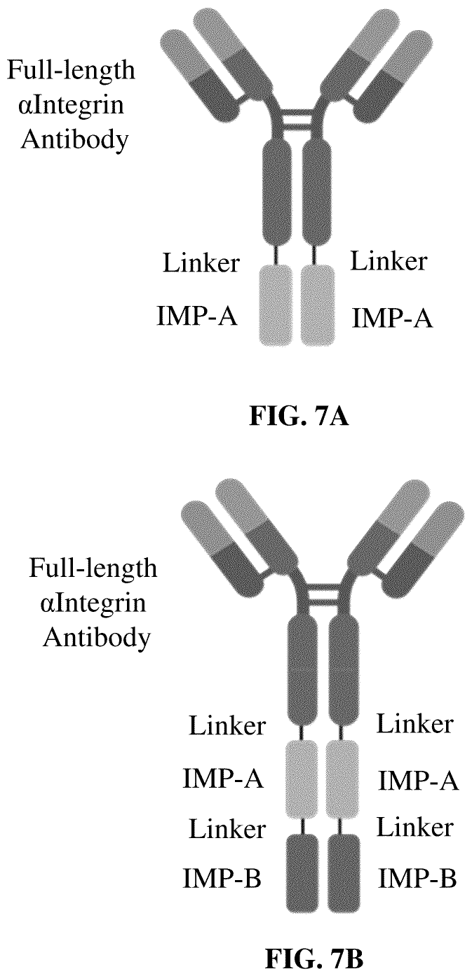

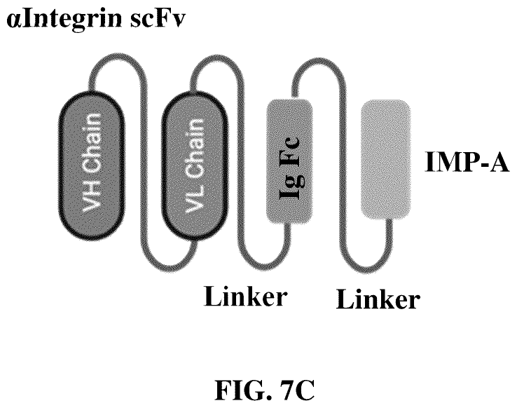

In some embodiments, the N-terminus of a first immunomodulatory protein described herein (see, e.g., §§ 5.2.1.1, § 5.2.1.2, 5.2.1.3, and 5.2.1.4) is operably connected (either directly or indirectly through a peptide linker) to the C-terminus of the first CH3 region of a full-length antibody that specifically binds an integrin (e.g., α4β7 integrin) (see, e.g., § 5.2.2.1), and the N-terminus of a second immunomodulatory protein described herein (see, e.g., §§ 5.2.1.1, § 5.2.1.2, 5.2.1.3, and 5.2.1.4) is operably connected (either directly or indirectly through a peptide linker) to the C-terminus the second CH3 region of the full length antibody. See, e.g., FIG. 7A. In some embodiments, the first immunomodulatory protein and the second immunomodulatory protein specifically bind the same target. In some embodiments, the amino acid sequence of the first immunomodulatory protein and the second immunomodulatory is 100% identical. In some embodiments, the first immunomodulatory protein and the second immunomodulatory protein specifically bind different targets. For example, in some embodiments, the first immunomodulatory protein specifically binds TL1A (e.g., proteins described in Table 3); the; and the second immunomodulatory protein specifically binds TNFα (e.g., proteins described in Table 4).

In some embodiments, the N-terminus of a first immunomodulatory protein described herein (see, e.g., §§ 5.2.1.1, § 5.2.1.2, 5.2.1.3, and 5.2.1.4) is operably connected (either directly or indirectly through a peptide linker) to the C-terminus of the first CH3 region of a full-length antibody that specifically binds an integrin (e.g., α4β7 integrin) (see, e.g., § 5.2.2.1), the N-terminus of a second immunomodulatory protein described herein (see, e.g., §§ 5.2.1.1, § 5.2.1.2, 5.2.1.3, and 5.2.1.4) is operably connected (either directly or indirectly through a peptide linker) to the C-terminus the first immunomodulatory protein; the N-terminus of a third immunomodulatory protein described herein (see, e.g., §§ 5.2.1.1, § 5.2.1.2, 5.2.1.3, and 5.2.1.4) is operably connected (either directly or indirectly through a peptide linker) to the C-terminus of the second CH3 region of the full-length antibody, and the N-terminus of a fourth immunomodulatory protein described herein (see, e.g., §§ 5.2.1.1, § 5.2.1.2, 5.2.1.3, and 5.2.1.4) is operably connected (either directly or indirectly through a peptide linker) to the C-terminus the third immunomodulatory protein. See, e.g., FIG. 7B. In some embodiments, the first immunomodulatory protein and the third immunomodulatory proteins specifically bind the same first target; and the second immunomodulatory protein and the fourth immunomodulatory proteins specifically bind the same second target, wherein the first and the second target are different. In some embodiments, the amino acid sequence of the first and the third immunomodulatory proteins are 100% identical; and the amino acid sequence of the third and the fourth immunomodulatory proteins are 100% identical. In some embodiments, the first and the third immunomodulatory proteins specifically bind TL1A (e.g., proteins described in Table 3); and the second and the fourth immunomodulatory protein specifically binds TNFα (e.g., proteins described in Table 4). In some embodiments, the first and the third immunomodulatory proteins specifically bind; and the second and the fourth immunomodulatory protein specifically bind TL1A (e.g., proteins described in Table 3).

In some embodiments, the fusion protein comprises a scFv that specifically binds an integrin (e.g., α4β7 integrin) (e.g., described herein) operably connected (either directly or indirectly through a peptide linker) to an Ig Fc region and an immunomodulatory protein described herein (see, e.g., §§ 5.2.1.1, § 5.2.1.2, 5.2.1.3, and 5.2.1.4) operably connected (either directly or indirectly through a peptide linker) to Ig Fc region. See, e.g., FIG. 7C. For example, in some embodiments, the N-terminus of an Ig Fc region operably connected (either directly or indirectly through a peptide linker) to the C-terminus of a scFv that specifically binds an integrin (e.g., described herein), and the N-terminus of an immunomodulatory protein described herein (see, e.g., §§ 5.2.1.1, § 5.2.1.2, 5.2.1.3, and 5.2.1.4) operably connected (either directly or indirectly through a peptide linker) to Ig Fc region. See, e.g., FIG. 7C.

### 5.7.3 Multimeric Fusion Proteins

In some embodiments, the fusion proteins described herein are multimeric (e.g., dimeric). In some embodiments, the fusion proteins described herein are multimeric (e.g., dimeric) comprising at least two fusion proteins described herein. In some embodiments, the multimeric (e.g., dimeric) fusion protein is homodimeric. In some embodiments, the multimeric (e.g., dimeric) fusion protein is heterodimeric. In some embodiments, the polypeptides of the multimeric (e.g., dimeric) fusion protein associate via one or more covalent or non-covalent interaction. In some embodiments, the polypeptides of the multimeric (e.g., dimeric) fusion protein associate via at least one covalent interaction. In some embodiments, the polypeptides of the multimeric (e.g., dimeric) fusion protein associate via one or more disulfide bond. In some embodiments, the polypeptides of the multimeric (e.g., dimeric) fusion protein associate via at least 1 disulfide bonds. In some embodiments, the polypeptides of the multimeric (e.g., dimeric) fusion protein associate via 1, 2, 3, 4, 5, 6, 7, 8, 9, 10, or more disulfide bonds.

In some embodiments, the multimeric fusion protein is dimeric comprising a first fusion protein (e.g., a hIg Fc fusion protein) described herein and a second fusion protein (e.g., an Ig (e.g., hIg, mIg) Fc fusion protein) described herein, wherein the amino acid sequence of the first fusion protein comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second fusion protein. For example, the amino acid sequence of the first fusion protein may comprise an amino acid sequence at least about 85% identical to the amino acid sequence of the second fusion protein. For example, the amino acid sequence of the first fusion protein may comprise an amino acid sequence at least about 90% identical to the amino acid sequence of the second fusion protein. For example, the amino acid sequence of the first fusion protein may comprise an amino acid sequence at least about 95% identical to the amino acid sequence of the second fusion protein. For example, the amino acid sequence of the first fusion protein may comprise an amino acid sequence at least about 99% identical to the amino acid sequence of the second fusion protein. In some embodiments, the amino acid sequence of the first fusion protein may preferably comprise an amino acid sequence 100% identical to the amino acid sequence of the second fusion protein.

In some embodiments, the multimeric fusion protein is dimeric comprising a first Ig (e.g., hIg) Fc fusion protein and a second Ig (e.g., hIg) Fc fusion protein. In some embodiments, the dimeric protein is homodimeric. In some embodiments, the amino acid sequence of the first Ig (e.g., hIg) Fc fusion protein comprises an amino acid sequence at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the second Ig (e.g., hIg) Fc fusion protein. In some embodiments, the amino acid sequence of the first Ig (e.g., hIg) Fc fusion protein comprises an amino acid sequence 100% identical to the amino acid sequence of the second Ig (e.g., hIg) Fc fusion protein.

An exemplary dimeric Ig (e.g., hIg) Fc fusion protein comprises, for example, a first fusion protein comprising a first immunomodulatory fusion protein, an Ig (e.g., hIg) Fc region, and a second immunomodulatory fusion protein, wherein the C-terminus of the first immunomodulatory protein described herein (see, e.g., §§ 5.2.1.1, 5.2.1.2, 5.2.1.3, and 5.2.1.4) is operably connected (either directly or through a peptide linker) to the N-terminus of the Ig (e.g., hIg) Fc region, and the N-terminus of the second immunomodulatory protein described herein (see, e.g., §§ 5.2.1.1, 5.2.1.2, 5.2.1.3, and 5.2.1.4) is operably connected (either directly or indirectly through a peptide linker) to the C-terminus of the Ig (e.g., hIg) Fc region; and a second fusion protein comprising the first immunomodulatory fusion protein, the Ig (e.g., hIg) Fc region, and the second immunomodulatory fusion protein, wherein the C-terminus of the first immunomodulatory protein described herein (see, e.g., §§ 5.2.1.1, 5.2.1.2, 5.2.1.3, and 5.2.1.4) is operably connected (either directly or indirectly through a peptide linker) to the N-terminus of the Ig (e.g., hIg) Fc region, and the N-terminus of the second immunomodulatory protein described herein (see, e.g., §§ 5.2.1.1, 5.2.1.2, 5.2.1.3, and 5.2.1.4) is operably connected (either directly or directly through a peptide linker) to the C-terminus of the Ig (e.g., hIg) Fc region. See, e.g., FIG. 1A. In some embodiments, the amino acid sequence of the first fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the fusion protein. In some embodiments, the amino acid sequence of the first fusion protein is 100% identical to the amino acid sequence of the fusion protein. In preferred embodiments, the first and second immunomodulatory proteins specifically bind different targets.

Another exemplary dimeric Ig (e.g., hIg) Fc fusion protein comprises, for example, a first fusion protein comprising a first immunomodulatory fusion protein, an Ig (e.g., hIg) Fc region, and a second immunomodulatory fusion protein, wherein the C-terminus of the first immunomodulatory protein described herein (see, e.g., §§ 5.2.1.1, 5.2.1.2, 5.2.1.3, and 5.2.1.4) is operably connected indirectly through a peptide linker to the N-terminus of the Ig (e.g., hIg) Fc region, and the N-terminus of the second immunomodulatory protein described herein (see, e.g., §§ 5.2.1.1, 5.2.1.2, 5.2.1.3, and 5.2.1.4) is operably connected indirectly through a peptide linker to the C-terminus of the Ig (e.g., hIg) Fc region; and a second fusion protein comprising the first immunomodulatory fusion protein, the Ig (e.g., hIg) Fc region, and the second immunomodulatory fusion protein, wherein the C-terminus of the first immunomodulatory protein described herein (see, e.g., §§ 5.2.1.1, 5.2.1.2, 5.2.1.3, and 5.2.1.4) is operably connected indirectly through a peptide linker to the N-terminus of the Ig (e.g., hIg) Fc region, and the N-terminus of the second immunomodulatory protein described herein (see, e.g., §§ 5.2.1.1, 5.2.1.2, 5.2.1.3, and 5.2.1.4) is operably connected (either directly or directly through a peptide linker) to the C-terminus of the Ig (e.g., hIg) Fc region. See, e.g., FIG. 1A. In some embodiments, the amino acid sequence of the first fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the fusion protein. In some embodiments, the amino acid sequence of the first fusion protein is 100% identical to the amino acid sequence of the fusion protein. In preferred embodiments, the first and second immunomodulatory proteins specifically bind different targets.

Another exemplary dimeric fusion protein comprises for example, a first fusion protein comprising a first immunomodulatory fusion protein, an Ig (e.g., hIg) Fc region, and a second immunomodulatory fusion protein, wherein the N-terminus of the first immunomodulatory protein described herein (see, e.g., §§ 5.2.1.1, 5.2.1.2, 5.2.1.3, and 5.2.1.4) is operably connected (either directly or indirectly through a peptide linker) to the C-terminus of the Ig (e.g., hIg) Fc region, and the N-terminus of the second immunomodulatory protein described herein (see, e.g., §§ 5.2.1.1, 5.2.1.2, 5.2.1.3, and 5.2.1.4) is operably connected (either directly or indirectly through a peptide linker) to the C-terminus of the first immunomodulatory fusion protein; and a second fusion protein comprising the first immunomodulatory fusion protein, the Ig (e.g., hIg) Fc region, and the second immunomodulatory fusion protein, wherein the C-terminus of the first immunomodulatory protein described herein (see, e.g., §§ 5.2.1.1, 5.2.1.2, 5.2.1.3, and 5.2.1.4) is operably connected (either directly or indirectly through a peptide linker) to the C-terminus of the Ig (e.g., hIg) Fc region, and the N-terminus of the second immunomodulatory protein described herein (see, e.g., §§ 5.2.1.1, 5.2.1.2, 5.2.1.3, and 5.2.1.4) is operably connected (either directly or indirectly through a peptide linker) to the C-terminus of the first immunomodulatory protein. In some embodiments, the amino acid sequence of the first fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the fusion protein. In some embodiments, the amino acid sequence of the first fusion protein is 100% identical to the amino acid sequence of the fusion protein. In preferred embodiments, the first and second immunomodulatory proteins specifically bind different targets. See, e.g., FIG. 1B.

Another exemplary dimeric fusion protein comprises for example, a first fusion protein comprising a first immunomodulatory fusion protein, an Ig (e.g., hIg) Fc region, and a second immunomodulatory fusion protein, wherein the N-terminus of the first immunomodulatory protein described herein (see, e.g., §§ 5.2.1.1, 5.2.1.2, 5.2.1.3, and 5.2.1.4) is operably connected indirectly through a peptide linker to the C-terminus of the Ig (e.g., hIg) Fc region, and the N-terminus of the second immunomodulatory protein described herein (see, e.g., §§ 5.2.1.1, 5.2.1.2, 5.2.1.3, and 5.2.1.4) is operably connected indirectly through a peptide linker to the C-terminus of the first immunomodulatory fusion protein; and a second fusion protein comprising the first immunomodulatory fusion protein, the Ig (e.g., hIg) Fc region, and the second immunomodulatory fusion protein, wherein the C-terminus of the first immunomodulatory protein described herein (see, e.g., §§ 5.2.1.1, 5.2.1.2, 5.2.1.3, and 5.2.1.4) is operably connected indirectly through a peptide linker to the C-terminus of the Ig (e.g., hIg) Fc region, and the N-terminus of the second immunomodulatory protein described herein (see, e.g., §§ 5.2.1.1, 5.2.1.2, 5.2.1.3, and 5.2.1.4) is operably connected indirectly through a peptide linker to the C-terminus of the first immunomodulatory protein. In some embodiments, the amino acid sequence of the first fusion protein is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of the fusion protein. In some embodiments, the amino acid sequence of the first fusion protein is 100% identical to the amino acid sequence of the fusion protein. In preferred embodiments, the first and second immunomodulatory proteins specifically bind different targets. See, e.g., FIG. 1B.

Another exemplary dimeric fusion protein comprises a full-length antibody, wherein the N-terminus of a first immunomodulatory protein is operably connected (either directly or indirectly through a peptide linker) to the C-terminus of the CH3 region of one of the heavy chains of the full-length antibody, and the N-terminus of a second immunomodulatory protein is operably connected (either directly or indirectly through a peptide linker) to the C-terminus of the first immunomodulatory protein; and wherein the N-terminus of the first immunomodulatory protein is operably connected (either directly or indirectly through a peptide linker) to the C-terminus of the CH3 region of the other the heavy chains of the full-length antibody, and the N-terminus of a second immunomodulatory protein is operably connected (either directly or indirectly through a peptide linker) to the C-terminus of the first immunomodulatory protein.

Another exemplary dimeric fusion protein comprises a full-length antibody, wherein the N-terminus of a first immunomodulatory protein is operably connected indirectly through a peptide linker to the C-terminus of the CH3 region of one of the heavy chains of the full-length antibody, and the N-terminus of a second immunomodulatory protein is operably connected indirectly through a peptide linker to the C-terminus of the first immunomodulatory protein; and wherein the N-terminus of the first immunomodulatory protein is operably connected indirectly through a peptide linker to the C-terminus of the CH3 region of the other the heavy chains of the full-length antibody, and the N-terminus of a second immunomodulatory protein is operably connected indirectly through a peptide linker to the C-terminus of the first immunomodulatory protein.

In some embodiments, the fusion proteins described herein are multimeric (e.g., dimeric, tetrameric (e.g., a full-length antibody)). In some embodiments, the fusion proteins described herein are multimeric comprising at least two fusion proteins described herein. In some embodiments, the multimeric fusion protein is homodimeric. In some embodiments, the multimeric fusion protein is heterodimeric. In some embodiments, the polypeptides of the multimeric fusion protein associate via one or more covalent or non-covalent interaction. In some embodiments, the polypeptides of the multimeric fusion protein associate via at least one covalent interaction. In some embodiments, the polypeptides of the multimeric fusion protein associate via one or more disulfide bond. In some embodiments, the polypeptides of the multimeric fusion protein associate via at least 1 disulfide bonds. In some embodiments, the polypeptides of the multimeric fusion protein associate via 1, 2, 3, 4, 5, 6, 7, 8, 9, 10, or more disulfide bonds.

For example, an exemplary multimeric fusion protein comprises a full-length antibody, wherein the N-terminus of a first immunomodulatory protein is operably connected (either directly or indirectly through a peptide linker) to the C-terminus of the CH3 region of one of the heavy chains of the full-length antibody, and the N-terminus of a second immunomodulatory protein is operably connected (either directly or indirectly through a peptide linker) to the C-terminus of the first immunomodulatory protein; and wherein the N-terminus of the first immunomodulatory protein is operably connected (either directly or indirectly through a peptide linker) to the C-terminus of the CH3 region of the other the heavy chains of the full-length antibody, and the N-terminus of a second immunomodulatory protein is operably connected (either directly or indirectly through a peptide linker) to the C-terminus of the first immunomodulatory protein.

Another exemplary multimeric fusion protein comprises a full-length antibody, wherein the N-terminus of a first immunomodulatory protein is operably connected indirectly through a peptide linker to the C-terminus of the CH3 region of one of the heavy chains of the full-length antibody, and the N-terminus of a second immunomodulatory protein is operably connected indirectly through a peptide linker to the C-terminus of the first immunomodulatory protein; and wherein the N-terminus of the first immunomodulatory protein is operably connected indirectly through a peptide linker to the C-terminus of the CH3 region of the other the heavy chains of the full-length antibody, and the N-terminus of a second immunomodulatory protein is operably connected indirectly through a peptide linker to the C-terminus of the first immunomodulatory protein.

### 5.8 Exemplary Immunomodulatory Fusion Proteins

The amino acid sequence of exemplary immunomodulatory fusion proteins (IFPs) described herein is provided in Table 16. The fusion proteins provided in Table 16 are exemplary only, and not intended to be limiting. Similar fusion proteins can be made utilizing the additional IMPs listed in Tables 2-5, e. g., an one of IMPs 1-275 (SEQ ID NOS: 34-576).

TABLE 16
The Amino Acid Sequence of Exemplary Immunomodulatory Fusion Proteins.

| | | | SEQ |
|---|---|---|---|
| Description | | Amino Acid Sequence | ID NO |
| IFP-1 | | MYRMQLLSCIALSLALVTNSSPGQGTQSENSCTHFPGNLPNML | 593 |
| with hIL-2 signal peptide | | RDLRDAFSRVKTFFQMKDQLDNLLLKESLLEDFKGYLGCQALS | |
| | | EMIQFYLEEVMPQAENQDPDIKAHVNSLGENLKTLRLRLRRCH | |
| | | RFLPCENKSKAVEQVKNAFNKLQEKGIYKAMSEFDIFINYIEA | |
| | | YMTMKIRNGGGGGGGSGGGGSGGGGSAESKYGPPCPPCPAPEA | |
| | | AGGPSVFLFPPKPKDTLMISRTPEVTCVVVDVSQEDPEVQFNW | |
| | | YVDGVEVHNAKTKPREEQFNSTYRVVSVLTVLHQDWLNGKEYK | |
| | | CKVSNKGLPSSIEKTISKAKGQPREPQVYTLPPSQEEMTKNQV | |
| | | SLTCLVKGFYPSDIAVEWESNGQPENNYKTTPPVLDSDGSFFL | |
| | | YSRLTVDKSRWQEGNVFSCSVMHEALHNHYTQKSLSLSLGGGG | |
| | | GGGGSGGGGSGGGGSDSKCGVSEYYNKEHDICCRLCPAGSYAE | |
| | | QLCTKDNDTVCNQCPPNTFLSIPNYISSCLSCRGKCINDHVED | |
| | | KPCTATSNRICKCKENKTCVLKTYDNSCRVCI | |
| IFP-1 | | SPGQGTQSENSCTHFPGNLPNMLRDLRDAFSRVKTFFQMKDQL | 594 |
| without hIL-2 signal | | DNLLLKESLLEDFKGYLGCQALSEMIQFYLEEVMPQAENQDPD | |
| peptide | | IKAHVNSLGENLKTLRLRLRRCHRFLPCENKSKAVEQVKNAFN | |
| | | KLQEKGIYKAMSEFDIFINYIEAYMTMKIRNGGGGGGGSGGGG | |
| | | SGGGGSAESKYGPPCPPCPAPEAAGGPSVFLFPPKPKDTLMIS | |
| | | RTPEVTCVVVDVSQEDPEVQFNWYVDGVEVHNAKTKPREEQFN | |
| | | STYRVVSVLTVLHQDWLNGKEYKCKVSNKGLPSSIEKTISKAK | |
| | | GQPREPQVYTLPPSQEEMTKNQVSLTCLVKGFYPSDIAVEWES | |
| | | NGQPENNYKTTPPVLDSDGSFFLYSRLTVDKSRWQEGNVFSCS | |
| | | VMHEALHNHYTQKSLSLSLGGGGGGGGSGGGGSGGGGSDSKCG | |
| | | VSEYYNKEHDICCRLCPAGSYAEQLCTKDNDTVCNQCPPNTFL | |
| | | SIPNYISSCLSCRGKCINDHVEDKPCTATSNRICKCKENKTCV | |
| | | LKTYDNSCRVCI | |
| IFP-2 | | MYRMQLLSCIALSLALVTNSKGRDSKPSPACDPMHGALAGIFK | 595 |
| with hIL-2 signal peptide | | ELRTTYRSVREALQTKDTVYYVSLFHEQLLQEMLSPVGCRVTN | |
| | | ELMQHYLDGVLPRAFHCGYDNATLNALHALSSSLSTLYQHMLK | |
| | | CPALACTGQTPAWTQFLDTEHKLDPWKGTVKATAEMDLLLNYL | |
| | | ETFLLQSGGGGGGGSGGGGSGGGGSAESKYGPPCPPCPAPEAA | |
| | | GGPSVFLFPPKPKDTLMISRTPEVTCVVVDVSQEDPEVQFNWY | |
| | | VDGVEVHNAKTKPREEQFNSTYRVVSVLTVLHQDWLNGKEYKC | |
| | | KVSNKGLPSSIEKTISKAKGQPREPQVYTLPPSQEEMTKNQVS | |
| | | LTCLVKGFYPSDIAVEWESNGQPENNYKTTPPVLDSDGSFFLY | |
| | | SRLTVDKSRWQEGNVFSCSVMHEALHNHYTQKSLSLSLGGGGG | |
| | | GGGSGGGGSGGGGSDSKCGVSEYYNKEHDICCRLCPAGSYAEQ | |
| | | LCTKDNDTVCNQCPPNTFLSIPNYISSCLSCRGKCINDHVEDK | |
| | | PCTATSNRICKCKENKTCVLKTYDNSCRVCI | |
| IFP-2 | | KGRDSKPSPACDPMHGALAGIFKELRTTYRSVREALQTKDTVY | 596 |
| without hIL-2 signal | | YVSLFHEQLLQEMLSPVGCRVTNELMQHYLDGVLPRAFHCGYD | |
| peptide | | NATLNALHALSSSLSTLYQHMLKCPALACTGQTPAWTQFLDTE | |
| | | HKLDPWKGTVKATAEMDLLLNYLETFLLQSGGGGGGGSGGGGS | |
| | | GGGGSAESKYGPPCPPCPAPEAAGGPSVFLFPPKPKDTLMISR | |
| | | TPEVTCVVVDVSQEDPEVQFNWYVDGVEVHNAKTKPREEQFNS | |
| | | TYRVVSVLTVLHQDWLNGKEYKCKVSNKGLPSSIEKTISKAKG | |
| | | QPREPQVYTLPPSQEEMTKNQVSLTCLVKGFYPSDIAVEWESN | |
| | | GQPENNYKTTPPVLDSDGSFFLYSRLTVDKSRWQEGNVFSCSV | |
| | | MHEALHNHYTQKSLSLSLGGGGGGGGSGGGGSGGGGSDSKCGV | |
| | | SEYYNKEHDICCRLCPAGSYAEQLCTKDNDTVCNQCPPNTFLS | |
| | | IPNYISSCLSCRGKCINDHVEDKPCTATSNRICKCKENKTCVL | |
| | | KTYDNSCRVCI | |
| IFP-3 | | MYRMQLLSCIALSLALVTNSSTYRVRSSGLTCSTCPPGTHKER | 597 |
| with hIL-2 signal peptide | | DCSLNTETICKACGEGEYTAHNNSLPKCLACKSCFNATEIETK | |
| | | SCDPTSDTICACREGYSINNLGECNGGGGGGGSGGGGSGGGGS | |
| | | AESKYGPPCPPCPAPEAAGGPSVFLFPPKPKDTLMISRTPEVT | |
| | | CVVVDVSQEDPEVQFNWYVDGVEVHNAKTKPREEQFNSTYRVV | |
| | | SVLTVLHQDWLNGKEYKCKVSNKGLPSSIEKTISKAKGQPREP | |
| | | QVYTLPPSQEEMTKNQVSLTCLVKGFYPSDIAVEWESNGQPEN | |
| | | NYKTTPPVLDSDGSFFLYSRLTVDKSRWQEGNVFSCSVMHEAL | |
| | | HNHYTQKSLSLSLGGGGGGGGSGGGGSGGGGSDSKCGVSEYYN | |
| | | KEHDICCRLCPAGSYAEQLCTKDNDTVCNQCPPNTFLSIPNYI | |
| | | SSCLSCRGKCINDHVEDKPCTATSNRICKCKENKTCVLKTYDN | |
| | | SCRVCI | |
| IFP-3 | | STYRVRSSGLTCSTCPPGTHKERDCSLNTETICKACGEGEYTA | 598 |
| without hIL-2 signal | | HNNSLPKCLACKSCFNATEIETKSCDPTSDTICACREGYSINN | |
| peptide | | LGECNGGGGGGGSGGGGSGGGGSAESKYGPPCPPCPAPEAAGG | |
| | | PSVFLFPPKPKDTLMISRTPEVTCVVVDVSQEDPEVQFNWYVD | |
| | | GVEVHNAKTKPREEQFNSTYRVVSVLTVLHQDWLNGKEYKCKV | |
| | | SNKGLPSSIEKTISKAKGQPREPQVYTLPPSQEEMTKNQVSLT | |
| | | CLVKGFYPSDIAVEWESNGQPENNYKTTPPVLDSDGSFFLYSR | |
| | | LTVDKSRWQEGNVFSCSVMHEALHNHYTQKSLSLSLGGGGGGG | |
| | | GSGGGGSGGGGSDSKCGVSEYYNKEHDICCRLCPAGSYAEQLC | |
| | | TKDNDTVCNQCPPNTFLSIPNYISSCLSCRGKCINDHVEDKPC | |
| | | TATSNRICKCKENKTCVLKTYDNSCRVCI | |
| IFP-4 | | MYRMQLLSCIALSLALVTNSSTYRVRSSGLTCSTCPPGTHKER | 599 |
| with hIL-2 signal peptide | | DCSLNTETICKACGEGEYTAHNNSLPKCLACKSCFNATEIETK | |
| | | SCDPTSDTICACREGYSINNLGECNGGGGGGGSGGGGSGGGGS | |
| | | AESKYGPPCPPCPAPEAAGGPSVFLFPPKPKDTLMISRTPEVT | |
| | | CVVVDVSQEDPEVQFNWYVDGVEVHNAKTKPREEQFNSTYRVV | |
| | | SVLTVLHQDWLNGKEYKCKVSNKGLPSSIEKTISKAKGQPREP | |
| | | QVYTLPPSQEEMTKNQVSLTCLVKGFYPSDIAVEWESNGQPEN | |
| | | NYKTTPPVLDSDGSFFLYSRLTVDKSRWQEGNVFSCSVMHEAL | |
| | | HNHYTQKSLSLSLGGGGGGGGSGGGGSGGGGSSTYRVRSSGLT | |
| | | CSTCPPGTHKERDCSLNTETICKACGEGEYTAHNNSLPKCLAC | |
| | | KSCFNATEIETKSCDPTSDTICACREGYSINNLGECN | |
| IFP-4 | | STYRVRSSGLTCSTCPPGTHKERDCSLNTETICKACGEGEYTA | 600 |
| without hIL-2 signal | | HNNSLPKCLACKSCENATEIETKSCDPTSDTICACREGYSINN | |
| peptide | | LGECNGGGGGGGSGGGGSGGGGSAESKYGPPCPPCPAPEAAGG | |
| | | PSVFLFPPKPKDTLMISRTPEVTCVVVDVSQEDPEVQFNWYVD | |
| | | GVEVHNAKTKPREEQFNSTYRVVSVLTVLHQDWLNGKEYKCKV | |
| | | SNKGLPSSIEKTISKAKGQPREPQVYTLPPSQEEMTKNQVSLT | |
| | | CLVKGFYPSDIAVEWESNGQPENNYKTTPPVLDSDGSFFLYSR | |
| | | LTVDKSRWQEGNVFSCSVMHEALHNHYTQKSLSLSLGGGGGGG | |
| | | GSGGGGSGGGGSSTYRVRSSGLTCSTCPPGTHKERDCSLNTET | |
| | | ICKACGEGEYTAHNNSLPKCLACKSCFNATEIETKSCDPTSDT | |
| | | ICACREGYSINNLGECN | |
| IFP-5 | | MYRMQLLSCIALSLALVTNSDSKCGVSEYYNKEHDICCRLCPA | 601 |
| with hIL-2 signal peptide | | GSYAEQLCTKDNDTVCNQCPPNTFLSIPNYISSCLSCRGKCIN | |
| | | DHVEDKPCTATSNRICKCKENKTCVLKTYDNSCRVCIGGGGGG | |
| | | GSGGGGSGGGGSAESKYGPPCPPCPAPEAAGGPSVFLFPPKPK | |
| | | DTLMISRTPEVTCVVVDVSQEDPEVQFNWYVDGVEVHNAKTKP | |
| | | REEQFNSTYRVVSVLTVLHQDWLNGKEYKCKVSNKGLPSSIEK | |
| | | TISKAKGQPREPQVYTLPPSQEEMTKNQVSLTCLVKGFYPSDI | |
| | | AVEWESNGQPENNYKTTPPVLDSDGSFFLYSRLTVDKSRWQEG | |
| | | NVFSCSVMHEALHNHYTQKSLSLSLGGGGGGGGSGGGGSGGGG | |
| | | SDSKCGVSEYYNKEHDICCRLCPAGSYAEQLCTKDNDTVCNQC | |
| | | PPNTFLSIPNYISSCLSCRGKCINDHVEDKPCTATSNRICKCK | |
| | | ENKTCVLKTYDNSCRVCI | |
| IFP-5 | | DSKCGVSEYYNKEHDICCRLCPAGSYAEQLCTKDNDTVCNQCP | 602 |
| without hIL-2 signal | | PNTFLSIPNYISSCLSCRGKCINDHVEDKPCTATSNRICKCKE | |
| peptide | | NKTCVLKTYDNSCRVCIGGGGGGGSGGGGSGGGGSAESKYGPP | |
| | | CPPCPAPEAAGGPSVFLFPPKPKDTLMISRTPEVTCVVVDVSQ | |
| | | EDPEVQFNWYVDGVEVHNAKTKPREEQFNSTYRVVSVLTVLHQ | |
| | | DWLNGKEYKCKVSNKGLPSSIEKTISKAKGQPREPQVYTLPPS | |
| | | QEEMTKNQVSLTCLVKGFYPSDIAVEWESNGQPENNYKTTPPV | |
| | | LDSDGSFFLYSRLTVDKSRWQEGNVFSCSVMHEALHNHYTQKS | |
| | | LSLSLGGGGGGGGSGGGGSGGGGSDSKCGVSEYYNKEHDICCR | |
| | | LCPAGSYAEQLCTKDNDTVCNQCPPNTFLSIPNYISSCLSCRG | |
| | | KCINDHVEDKPCTATSNRICKCKENKTCVLKTYDNSCRVCI | |
| IFP-6 | | MYRMQLLSCIALSLALVTNSAESKYGPPCPPCPAPEAAGGPSV | 603 |
| with hIL-2 signal peptide | | FLFPPKPKDTLMISRTPEVTCVVVDVSQEDPEVQFNWYVDGVE | |
| | | VHNAKTKPREEQFNSTYRVVSVLTVLHQDWLNGKEYKCKVSNK | |
| | | GLPSSIEKTISKAKGQPREPQVYTLPPSQEEMTKNQVSLTCLV | |
| | | KGFYPSDIAVEWESNGQPENNYKTTPPVLDSDGSFFLYSRLTV | |
| | | DKSRWQEGNVFSCSVMHEALHNHYTQKSLSLSLGGGGGGGGSG | |
| | | GGGSGGGGSSTYRVRSSGLTCSTCPPGTHKERDCSLNTETICK | |
| | | ACGEGEYTAHNNSLPKCLACKSCFNATEIETKSCDPTSDTICA | |
| | | CREGYSINNLGECNGGGGGGGGSGGGGSGGGGSSTYRVRSSGL | |
| | | TCSTCPPGTHKERDCSLNTETICKACGEGEYTAHNNSLPKCLA | |
| | | CKSCFNATEIETKSCDPTSDTICACREGYSINNLGECN | |
| IFP-6 | | AESKYGPPCPPCPAPEAAGGPSVFLFPPKPKDTLMISRTPEVT | 604 |
| without hIL-2 signal | | CVVVDVSQEDPEVQFNWYVDGVEVHNAKTKPREEQFNSTYRVV | |
| peptide | | SVLTVLHQDWLNGKEYKCKVSNKGLPSSIEKTISKAKGQPREP | |
| | | QVYTLPPSQEEMTKNQVSLTCLVKGFYPSDIAVEWESNGQPEN | |
| | | NYKTTPPVLDSDGSFFLYSRLTVDKSRWQEGNVFSCSVMHEAL | |
| | | HNHYTQKSLSLSLGGGGGGGGSGGGGSGGGGSSTYRVRSSGLT | |
| | | CSTCPPGTHKERDCSLNTETICKACGEGEYTAHNNSLPKCLAC | |
| | | KSCFNATEIETKSCDPTSDTICACREGYSINNLGECNGGGGGG | |
| | | GGSGGGGSGGGGSSTYRVRSSGLTCSTCPPGTHKERDCSLNTE | |
| | | TICKACGEGEYTAHNNSLPKCLACKSCFNATEIETKSCDPTSD | |
| | | TICACREGYSINNLGECN | |
| IFP-7 | | MYRMQLLSCIALSLALVTNSAESKYGPPCPPCPAPEAAGGPSV | 605 |
| with hIL-2 signal peptide | | FLFPPKPKDTLMISRTPEVTCVVVDVSQEDPEVQFNWYVDGVE | |
| | | VHNAKTKPREEQFNSTYRVVSVLTVLHQDWLNGKEYKCKVSNK | |
| | | GLPSSIEKTISKAKGQPREPQVYTLPPSQEEMTKNQVSLTCLV | |
| | | KGFYPSDIAVEWESNGQPENNYKTTPPVLDSDGSFFLYSRLTV | |
| | | DKSRWQEGNVFSCSVMHEALHNHYTQKSLSLSLGGGGGGGGSG | |
| | | GGGSGGGGSDSKCGVSEYYNKEHDICCRLCPAGSYAEQLCTKD | |
| | | NDTVCNQCPPNTFLSIPNYISSCLSCRGKCINDHVEDKPCTAT | |
| | | SNRICKCKENKTCVLKTYDNSCRVCIGGGGGGGGSGGGGSGGG | |
| | | GSDSKCGVSEYYNKEHDICCRLCPAGSYAEQLCTKDNDTVCNQ | |
| | | CPPNTFLSIPNYISSCLSCRGKCINDHVEDKPCTATSNRICKC | |
| | | KENKTCVLKTYDNSCRVCI | |
| IFP-7 | | AESKYGPPCPPCPAPEAAGGPSVFLFPPKPKDTLMISRTPEVT | 606 |
| without hIL-2 signal | | CVVVDVSQEDPEVQFNWYVDGVEVHNAKTKPREEQFNSTYRVV | |
| peptide | | SVLTVLHQDWLNGKEYKCKVSNKGLPSSIEKTISKAKGQPREP | |
| | | QVYTLPPSQEEMTKNQVSLTCLVKGFYPSDIAVEWESNGQPEN | |
| | | NYKTTPPVLDSDGSFFLYSRLTVDKSRWQEGNVESCSVMHEAL | |
| | | HNHYTQKSLSLSLGGGGGGGGSGGGGSGGGGSDSKCGVSEYYN | |
| | | KEHDICCRLCPAGSYAEQLCTKDNDTVCNQCPPNTFLSIPNYI | |
| | | SSCLSCRGKCINDHVEDKPCTATSNRICKCKENKTCVLKTYDN | |
| | | SCRVCIGGGGGGGGSGGGGSGGGGSDSKCGVSEYYNKEHDICC | |
| | | RLCPAGSYAEQLCTKDNDTVCNQCPPNTFLSIPNYISSCLSCR | |
| | | GKCINDHVEDKPCTATSNRICKCKENKTCVLKTYDNSCRVCI | |
| IFP-8 | | MYRMQLLSCIALSLALVTNSAESKYGPPCPPCPAPEAAGGPSV | 607 |
| with hIL-2 signal peptide | | FLFPPKPKDTLMISRTPEVTCVVVDVSQEDPEVQFNWYVDGVE | |
| | | VHNAKTKPREEQFNSTYRVVSVLTVLHQDWLNGKEYKCKVSNK | |
| | | GLPSSIEKTISKAKGQPREPQVYTLPPSQEEMTKNQVSLTCLV | |
| | | KGFYPSDIAVEWESNGQPENNYKTTPPVLDSDGSFFLYSRLTV | |
| | | DKSRWQEGNVFSCSVMHEALHNHYTQKSLSLSLGGGGGGGGSG | |
| | | GGGSGGGGSSTYRVRSSGLTCSTCPPGTHKERDCSLNTETICK | |
| | | ACGEGEYTAHNNSLPKCLACKSCFNATEIETKSCDPTSDTICA | |
| | | CREGYSINNLGECNGGGGGGGSGGGGSGGGGSDSKCGVSEYYN | |
| | | KEHDICCRLCPAGSYAEQLCTKDNDTVCNQCPPNTFLSIPNYI | |
| | | SSCLSCRGKCINDHVEDKPCTATSNRICKCKENKTCVLKTYDN | |
| | | SCRVCI | |
| IFP-8 | | AESKYGPPCPPCPAPEAAGGPSVFLFPPKPKDTLMISRTPEVT | 608 |
| without hIL-2 signal | | CVVVDVSQEDPEVQFNWYVDGVEVHNAKTKPREEQFNSTYRVV | |
| peptide | | SVLTVLHQDWLNGKEYKCKVSNKGLPSSIEKTISKAKGQPREP | |
| | | QVYTLPPSQEEMTKNQVSLTCLVKGFYPSDIAVEWESNGQPEN | |
| | | NYKTTPPVLDSDGSFFLYSRLTVDKSRWQEGNVFSCSVMHEAL | |
| | | HNHYTQKSLSLSLGGGGGGGGSGGGGSGGGGSSTYRVRSSGLT | |
| | | CSTCPPGTHKERDCSLNTETICKACGEGEYTAHNNSLPKCLAC | |
| | | KSCFNATEIETKSCDPTSDTICACREGYSINNLGECNGGGGGG | |
| | | GSGGGGSGGGGSDSKCGVSEYYNKEHDICCRLCPAGSYAEQLC | |
| | | TKDNDTVCNQCPPNTFLSIPNYISSCLSCRGKCINDHVEDKPC | |
| | | TATSNRICKCKENKTCVLKTYDNSCRVCI | |
| IFP-9 | | MYRMQLLSCIALSLALVTNSAESKYGPPCPPCPAPEAAGGPSV | 609 |
| with hIL-2 signal peptide | | FLFPPKPKDTLMISRTPEVTCVVVDVSQEDPEVQFNWYVDGVE | |
| | | VHNAKTKPREEQFNSTYRVVSVLTVLHQDWLNGKEYKCKVSNK | |
| | | GLPSSIEKTISKAKGQPREPQVYTLPPSQEEMTKNQVSLTCLV | |
| | | KGFYPSDIAVEWESNGQPENNYKTTPPVLDSDGSFFLYSRLTV | |
| | | DKSRWQEGNVFSCSVMHEALHNHYTQKSLSLSLGGGGGGGGSG | |
| | | GGGSGGGGSDSKCGVSEYYNKEHDICCRLCPAGSYAEQLCTKD | |
| | | NDTVCNQCPPNTFLSIPNYISSCLSCRGKCINDHVEDKPCTAT | |
| | | SNRICKCKENKTCVLKTYDNSCRVCIGGGGGGGSGGGGSGGGG | |
| | | SSTYRVRSSGLTCSTCPPGTHKERDCSLNTETICKACGEGEYT | |
| | | AHNNSLPKCLACKSCFNATEIETKSCDPTSDTICACREGYSIN | |
| | | NLGECN | |
| IFP-9 | | AESKYGPPCPPCPAPEAAGGPSVFLFPPKPKDTLMISRTPEVT | 610 |
| without hIL-2 signal | | CVVVDVSQEDPEVQFNWYVDGVEVHNAKTKPREEQFNSTYRVV | |
| peptide | | SVLTVLHQDWLNGKEYKCKVSNKGLPSSIEKTISKAKGQPREP | |
| | | QVYTLPPSQEEMTKNQVSLTCLVKGFYPSDIAVEWESNGQPEN | |
| | | NYKTTPPVLDSDGSFFLYSRLTVDKSRWQEGNVFSCSVMHEAL | |
| | | HNHYTQKSLSLSLGGGGGGGGSGGGGSGGGGSDSKCGVSEYYN | |
| | | KEHDICCRLCPAGSYAEQLCTKDNDTVCNQCPPNTFLSIPNYI | |
| | | SSCLSCRGKCINDHVEDKPCTATSNRICKCKENKTCVLKTYDN | |
| | | SCRVCIGGGGGGGSGGGGSGGGGSSTYRVRSSGLTCSTCPPGT | |
| | | HKERDCSLNTETICKACGEGEYTAHNNSLPKCLACKSCFNATE | |
| | | IETKSCDPTSDTICACREGYSINNLGECN | |
| IFP-10 | | MYRMQLLSCIALSLALVTNSDSKCGVSEYYNKEHDICCRLCPA | 611 |
| with hIL-2 signal peptide | | GSYAEQLCTKDNDTVCNQCPPNTFLSIPNYISSCLSCRGKCIN | |
| | | DHVEDKPCTATSNRICKCKENKTCVLKTYDNSCRVCIGGGGGG | |
| | | GSGGGGSGGGGSAESKYGPPCPPCPAPEAAGGPSVFLFPPKPK | |
| | | DTLMISRTPEVTCVVVDVSQEDPEVQFNWYVDGVEVHNAKTKP | |
| | | REEQFNSTYRVVSVLTVLHQDWLNGKEYKCKVSNKGLPSSIEK | |
| | | TISKAKGQPREPQVYTLPPSQEEMTKNQVSLTCLVKGFYPSDI | |
| | | AVEWESNGQPENNYKTTPPVLDSDGSFFLYSRLTVDKSRWQEG | |
| | | NVFSCSVMHEALHNHYTQKSLSLSLGGGGGGGGSGGGGSGGGG | |
| | | SSTYRVRSSGLTCSTCPPGTHKERDCSLNTETICKACGEGEYT | |
| | | AHNNSLPKCLACKSCFNATEIETKSCDPTSDTICACREGYSIN | |
| | | NLGECN | |
| IFP-10 | | DSKCGVSEYYNKEHDICCRLCPAGSYAEQLCTKDNDTVCNQCP | 612 |
| without hIL-2 signal | | PNTFLSIPNYISSCLSCRGKCINDHVEDKPCTATSNRICKCKE | |
| peptide | | NKTCVLKTYDNSCRVCIGGGGGGGSGGGGSGGGGSAESKYGPP | |
| | | CPPCPAPEAAGGPSVFLFPPKPKDTLMISRTPEVTCVVVDVSQ | |
| | | EDPEVQFNWYVDGVEVHNAKTKPREEQFNSTYRVVSVLTVLHQ | |
| | | DWLNGKEYKCKVSNKGLPSSIEKTISKAKGQPREPQVYTLPPS | |
| | | QEEMTKNQVSLTCLVKGFYPSDIAVEWESNGQPENNYKTTPPV | |
| | | LDSDGSFFLYSRLTVDKSRWQEGNVFSCSVMHEALHNHYTQKS | |
| | | LSLSLGGGGGGGGSGGGGSGGGGSSTYRVRSSGLTCSTCPPGT | |
| | | HKERDCSLNTETICKACGEGEYTAHNNSLPKCLACKSCFNATE | |
| | | IETKSCDPTSDTICACREGYSINNLGECN | |
| IFP-11 | | MYRMQLLSCIALSLALVTNSQVQLVQSGAEVKKPGASVKVSCK | 613 |
| with hIL-2 signal peptide | | GSGYTFTSYWMHWVRQAPGQRLEWIGEIDPSESNTNYNQKFKG | |
| | | RVTLTVDISASTAYMELSSLRSEDTAVYYCARGGYDGWDYAID | |
| | | YWGQGTLVTVSSASTKGPSVFPLAPSSKSTSGGTAALGCLVKD | |
| | | YFPEPVTVSWNSGALTSGVHTFPAVLQSSGLYSLSSVVTVPSS | |
| | | SLGTQTYICNVNHKPSNTKVDKKVEPKSCDKTHTCPPCPAPEL | |
| | | AGAPSVFLFPPKPKDTLMISRTPEVTCVVVDVSHEDPEVKFNW | |
| | | YVDGVEVHNAKTKPREEQYNSTYRVVSVLTVLHQDWLNGKEYK | |
| | | CKVSNKALPAPIEKTISKAKGQPREPQVYTLPPSRDELTKNQV | |
| | | SLTCLVKGFYPSDIAVEWESNGQPENNYKTTPPVLDSDGSFFL | |
| | | YSKLTVDKSRWQQGNVFSCSVMHEALHNHYTQKSLSLSPGKGG | |
| | | GGGGGSGGGGSGGGGSDVVMTQSPLSLPVTPGEPASISCRSSQ | |
| | | SLAKSYGNTYLSWYLQKPGQSPQLLIYGISNRFSGVPDRFSGS | |
| | | GSGTDFTLKISRVEAEDVGVYYCLQGTHQPYTFGQGTKVEIKR | |
| | | TVAAPSVFIFPPSDEQLKSGTASVVCLLNNFYPREAKVQWKVD | |
| | | NALQSGNSQESVTEQDSKDSTYSLSSTLTLSKADYEKHKVYAC | |
| | | EVTHQGLSSPVTKSFNRGECGGGGGGGSGGGGSGGGGSAESKY | |
| | | GPPCPPCPAPEAAGGPSVFLFPPKPKDTLMISRTPEVTCVVVD | |
| | | VSQEDPEVQFNWYVDGVEVHNAKTKPREEQFNSTYRVVSVLTV | |
| | | LHQDWLNGKEYKCKVSNKGLPSSIEKTISKAKGQPREPQVYTL | |
| | | PPSQEEMTKNQVSLTCLVKGFYPSDIAVEWESNGQPENNYKTT | |
| | | PPVLDSDGSFFLYSRLTVDKSRWQEGNVFSCSVMHEALHNHYT | |
| | | QKSLSLSLGGGGGGGGSGGGGSGGGGSDSKCGVSEYYNKEHDI | |
| | | CCRLCPAGSYAEQLCTKDNDTVCNQCPPNTFLSIPNYISSCLS | |
| | | CRGKCINDHVEDKPCTATSNRICKCKENKTCVLKTYDNSCRVC | |
| | | I | |
| IFP-11 | | QVQLVQSGAEVKKPGASVKVSCKGSGYTFTSYWMHWVRQAPGQ | 614 |
| without hIL-2 signal | | RLEWIGEIDPSESNTNYNQKFKGRVTLTVDISASTAYMELSSL | |
| peptide | | RSEDTAVYYCARGGYDGWDYAIDYWGQGTLVTVSSASTKGPSV | |
| | | FPLAPSSKSTSGGTAALGCLVKDYFPEPVTVSWNSGALTSGVH | |
| | | TFPAVLQSSGLYSLSSVVTVPSSSLGTQTYICNVNHKPSNTKV | |
| | | DKKVEPKSCDKTHTCPPCPAPELAGAPSVFLFPPKPKDTLMIS | |
| | | RTPEVTCVVVDVSHEDPEVKFNWYVDGVEVHNAKTKPREEQYN | |
| | | STYRVVSVLTVLHQDWLNGKEYKCKVSNKALPAPIEKTISKAK | |
| | | GQPREPQVYTLPPSRDELTKNQVSLTCLVKGFYPSDIAVEWES | |
| | | NGQPENNYKTTPPVLDSDGSFFLYSKLTVDKSRWQQGNVFSCS | |
| | | VMHEALHNHYTQKSLSLSPGKGGGGGGGSGGGGSGGGGSDVVM | |
| | | TQSPLSLPVTPGEPASISCRSSQSLAKSYGNTYLSWYLQKPGQ | |
| | | SPQLLIYGISNRFSGVPDRFSGSGSGTDFTLKISRVEAEDVGV | |
| | | YYCLQGTHQPYTFGQGTKVEIKRTVAAPSVFIFPPSDEQLKSG | |
| | | TASVVCLLNNFYPREAKVQWKVDNALQSGNSQESVTEQDSKDS | |
| | | TYSLSSTLTLSKADYEKHKVYACEVTHQGLSSPVTKSFNRGEC | |
| | | GGGGGGGSGGGGSGGGGSAESKYGPPCPPCPAPEAAGGPSVFL | |
| | | FPPKPKDTLMISRTPEVTCVVVDVSQEDPEVQFNWYVDGVEVH | |
| | | NAKTKPREEQFNSTYRVVSVLTVLHQDWLNGKEYKCKVSNKGL | |
| | | PSSIEKTISKAKGQPREPQVYTLPPSQEEMTKNQVSLTCLVKG | |
| | | FYPSDIAVEWESNGQPENNYKTTPPVLDSDGSFFLYSRLTVDK | |
| | | SRWQEGNVFSCSVMHEALHNHYTQKSLSLSLGGGGGGGGSGGG | |
| | | GSGGGGSDSKCGVSEYYNKEHDICCRLCPAGSYAEQLCTKDND | |
| | | TVCNQCPPNTFLSIPNYISSCLSCRGKCINDHVEDKPCTATSN | |
| | | RICKCKENKTCVLKTYDNSCRVCI | |
| IFP-12 | | MYRMQLLSCIALSLALVTNSQVQLVQSGAEVKKPGASVKVSCK | 615 |
| with hIL-2 signal peptide | | GSGYTFTSYWMHWVRQAPGQRLEWIGEIDPSESNTNYNQKFKG | |
| | | RVTLTVDISASTAYMELSSLRSEDTAVYYCARGGYDGWDYAID | |
| | | YWGQGTLVTVSSGGGGGGGSGGGGSGGGGSDVVMTQSPLSLPV | |
| | | TPGEPASISCRSSQSLAKSYGNTYLSWYLQKPGQSPQLLIYGI | |
| | | SNRFSGVPDRFSGSGSGTDFTLKISRVEAEDVGVYYCLQGTHQ | |
| | | PYTFGQGTKVEIKGGGGGGGSGGGGSGGGGSAESKYGPPCPPC | |
| | | PAPEAAGGPSVFLFPPKPKDTLMISRTPEVTCVVVDVSQEDPE | |
| | | VQFNWYVDGVEVHNAKTKPREEQFNSTYRVVSVLTVLHQDWLN | |
| | | GKEYKCKVSNKGLPSSIEKTISKAKGQPREPQVYTLPPSQEEM | |
| | | TKNQVSLTCLVKGFYPSDIAVEWESNGQPENNYKTTPPVLDSD | |
| | | GSFFLYSRLTVDKSRWQEGNVFSCSVMHEALHNHYTQKSLSLS | |
| | | LGGGGGGGGSGGGGSGGGGSDSKCGVSEYYNKEHDICCRLCPA | |
| | | GSYAEQLCTKDNDTVCNQCPPNTFLSIPNYISSCLSCRGKCIN | |
| | | DHVEDKPCTATSNRICKCKENKTCVLKTYDNSCRVCI | |
| IFP-12 | | QVQLVQSGAEVKKPGASVKVSCKGSGYTFTSYWMHWVRQAPGQ | 616 |
| without hIL-2 signal | | RLEWIGEIDPSESNTNYNQKFKGRVTLTVDISASTAYMELSSL | |
| peptide | | RSEDTAVYYCARGGYDGWDYAIDYWGQGTLVTVSSGGGGGGGS | |
| | | GGGGSGGGGSDVVMTQSPLSLPVTPGEPASISCRSSQSLAKSY | |
| | | GNTYLSWYLQKPGQSPQLLIYGISNRFSGVPDRFSGSGSGTDF | |
| | | TLKISRVEAEDVGVYYCLQGTHQPYTFGQGTKVEIKGGGGGGG | |
| | | SGGGGSGGGGSAESKYGPPCPPCPAPEAAGGPSVFLFPPKPKD | |
| | | TLMISRTPEVTCVVVDVSQEDPEVQFNWYVDGVEVHNAKTKPR | |
| | | EEQFNSTYRVVSVLTVLHQDWLNGKEYKCKVSNKGLPSSIEKT | |
| | | ISKAKGQPREPQVYTLPPSQEEMTKNQVSLTCLVKGFYPSDIA | |
| | | VEWESNGQPENNYKTTPPVLDSDGSFFLYSRLTVDKSRWQEGN | |
| | | VFSCSVMHEALHNHYTQKSLSLSLGGGGGGGGSGGGGSGGGGS | |
| | | DSKCGVSEYYNKEHDICCRLCPAGSYAEQLCTKDNDTVCNQCP | |
| | | PNTFLSIPNYISSCLSCRGKCINDHVEDKPCTATSNRICKCKE | |
| | | NKTCVLKTYDNSCRVCI | |
| IFP-13 | Heavy | MYRMQLLSCIALSLALVTNSQVQLVQSGAEVKKPGASVKVSCK | 617 |
| with hIL-2 | Chain | GSGYTFTSYWMHWVRQAPGQRLEWIGEIDPSESNTNYNQKFKG | |
| signal peptide | | RVTLTVDISASTAYMELSSLRSEDTAVYYCARGGYDGWDYAID | |
| | | YWGQGTLVTVSSASTKGPSVFPLAPSSKSTSGGTAALGCLVKD | |
| | | YFPEPVTVSWNSGALTSGVHTFPAVLQSSGLYSLSSVVTVPSS | |
| | | SLGTQTYICNVNHKPSNTKVDKKVEPKSCDKTHTCPPCPAPEL | |
| | | AGAPSVFLFPPKPKDTLMISRTPEVTCVVVDVSHEDPEVKFNW | |
| | | YVDGVEVHNAKTKPREEQYNSTYRVVSVLTVLHQDWLNGKEYK | |
| | | CKVSNKALPAPIEKTISKAKGQPREPQVYTLPPSRDELTKNQV | |
| | | SLTCLVKGFYPSDIAVEWESNGQPENNYKTTPPVLDSDGSFFL | |
| | | YSKLTVDKSRWQQGNVFSCSVMHEALHNHYTQKSLSLSPGKGG | |
| | | GGGGGSGGGGSGGGGSDSKCGVSEYYNKEHDICCRLCPAGSYA | |
| | | EQLCTKDNDTVCNQCPPNTFLSIPNYISSCLSCRGKCINDHVE | |
| | | DKPCTATSNRICKCKENKTCVLKTYDNSCRVCI | |
| | Light | DVVMTQSPLSLPVTPGEPASISCRSSQSLAKSYGNTYLSWYLQ | 592 |
| | Chain | KPGQSPQLLIYGISNRFSGVPDRFSGSGSGTDFTLKISRVEAE | |
| | | DVGVYYCLQGTHQPYTFGQGTKVEIKRTVAAPSVFIFPPSDEQ | |
| | | LKSGTASVVCLLNNFYPREAKVQWKVDNALQSGNSQESVTEQD | |
| | | SKDSTYSLSSTLTLSKADYEKHKVYACEVTHQGLSSPVTKSFN | |
| | | RGEC | |
| IFP-13 | Heavy | QVQLVQSGAEVKKPGASVKVSCKGSGYTFTSYWMHWVRQAPGQ | 618 |
| without hIL-2 | Chain | RLEWIGEIDPSESNTNYNQKFKGRVTLTVDISASTAYMELSSL | |
| signal peptide | | RSEDTAVYYCARGGYDGWDYAIDYWGQGTLVTVSSASTKGPSV | |
| | | FPLAPSSKSTSGGTAALGCLVKDYFPEPVTVSWNSGALTSGVH | |
| | | TFPAVLQSSGLYSLSSVVTVPSSSLGTQTYICNVNHKPSNTKV | |
| | | DKKVEPKSCDKTHTCPPCPAPELAGAPSVFLFPPKPKDTLMIS | |
| | | RTPEVTCVVVDVSHEDPEVKFNWYVDGVEVHNAKTKPREEQYN | |
| | | STYRVVSVLTVLHQDWLNGKEYKCKVSNKALPAPIEKTISKAK | |
| | | GQPREPQVYTLPPSRDELTKNQVSLTCLVKGFYPSDIAVEWES | |
| | | NGQPENNYKTTPPVLDSDGSFFLYSKLTVDKSRWQQGNVFSCS | |
| | | VMHEALHNHYTQKSLSLSPGKGGGGGGGSGGGGSGGGGSDSKC | |
| | | GVSEYYNKEHDICCRLCPAGSYAEQLCTKDNDTVCNQCPPNTF | |
| | | LSIPNYISSCLSCRGKCINDHVEDKPCTATSNRICKCKENKTC | |
| | | VLKTYDNSCRVCI | |
| | Light | DVVMTQSPLSLPVTPGEPASISCRSSQSLAKSYGNTYLSWYLQ | 592 |
| | Chain | KPGQSPQLLIYGISNRFSGVPDRFSGSGSGTDFTLKISRVEAE | |
| | | DVGVYYCLQGTHQPYTFGQGTKVEIKRTVAAPSVFIFPPSDEQ | |
| | | LKSGTASVVCLLNNFYPREAKVQWKVDNALQSGNSQESVTEQD | |
| | | SKDSTYSLSSTLTLSKADYEKHKVYACEVTHQGLSSPVTKSFN | |
| | | RGEC | |
| IFP-14 | Heavy | MYRMQLLSCIALSLALVTNSQVQLVQSGAEVKKPGASVKVSCK | 619 |
| with hIL-2 | Chain | GSGYTFTSYWMHWVRQAPGQRLEWIGEIDPSESNTNYNQKFKG | |
| signal peptide | | RVTLTVDISASTAYMELSSLRSEDTAVYYCARGGYDGWDYAID | |
| | | YWGQGTLVTVSSASTKGPSVFPLAPSSKSTSGGTAALGCLVKD | |
| | | YFPEPVTVSWNSGALTSGVHTFPAVLQSSGLYSLSSVVTVPSS | |
| | | SLGTQTYICNVNHKPSNTKVDKKVEPKSCDKTHTCPPCPAPEL | |
| | | AGAPSVFLFPPKPKDTLMISRTPEVTCVVVDVSHEDPEVKFNW | |
| | | YVDGVEVHNAKTKPREEQYNSTYRVVSVLTVLHQDWLNGKEYK | |
| | | CKVSNKALPAPIEKTISKAKGQPREPQVYTLPPSRDELTKNQV | |
| | | SLTCLVKGFYPSDIAVEWESNGQPENNYKTTPPVLDSDGSFFL | |
| | | YSKLTVDKSRWQQGNVFSCSVMHEALHNHYTQKSLSLSPGKGG | |
| | | GGGGGSGGGGSGGGGSSTYRVRSSGLTCSTCPPGTHKERDCSL | |
| | | NTETICKACGEGEYTAHNNSLPKCLACKSCFNATEIETKSCDP | |
| | | TSDTICACREGYSINNLGECN | |
| | Light | DVVMTQSPLSLPVTPGEPASISCRSSQSLAKSYGNTYLSWYLQ | 592 |
| | Chain | KPGQSPQLLIYGISNRFSGVPDRFSGSGSGTDFTLKISRVEAE | |
| | | DVGVYYCLQGTHQPYTFGQGTKVEIKRTVAAPSVFIFPPSDEQ | |
| | | LKSGTASVVCLLNNFYPREAKVQWKVDNALQSGNSQESVTEQD | |
| | | SKDSTYSLSSTLTLSKADYEKHKVYACEVTHQGLSSPVTKSFN | |
| | | RGEC | |
| IFP-14 | Heavy | QVQLVQSGAEVKKPGASVKVSCKGSGYTFTSYWMHWVRQAPGQ | 620 |
| without hIL-2 | Chain | RLEWIGEIDPSESNTNYNQKFKGRVTLTVDISASTAYMELSSL | |
| signal peptide | | RSEDTAVYYCARGGYDGWDYAIDYWGQGTLVTVSSASTKGPSV | |
| | | FPLAPSSKSTSGGTAALGCLVKDYFPEPVTVSWNSGALTSGVH | |
| | | TFPAVLQSSGLYSLSSVVTVPSSSLGTQTYICNVNHKPSNTKV | |
| | | DKKVEPKSCDKTHTCPPCPAPELAGAPSVFLFPPKPKDTLMIS | |
| | | RTPEVTCVVVDVSHEDPEVKFNWYVDGVEVHNAKTKPREEQYN | |
| | | STYRVVSVLTVLHQDWLNGKEYKCKVSNKALPAPIEKTISKAK | |
| | | GQPREPQVYTLPPSRDELTKNQVSLTCLVKGFYPSDIAVEWES | |
| | | NGQPENNYKTTPPVLDSDGSFFLYSKLTVDKSRWQQGNVFSCS | |
| | | VMHEALHNHYTQKSLSLSPGKGGGGGGGSGGGGSGGGGSSTYR | |
| | | VRSSGLTCSTCPPGTHKERDCSLNTETICKACGEGEYTAHNNS | |
| | | LPKCLACKSCFNATEIETKSCDPTSDTICACREGYSINNLGEC | |
| | | N | |
| | Light | DVVMTQSPLSLPVTPGEPASISCRSSQSLAKSYGNTYLSWYLQ | 592 |
| | Chain | KPGQSPQLLIYGISNRFSGVPDRFSGSGSGTDFTLKISRVEAE | |
| | | DVGVYYCLQGTHQPYTFGQGTKVEIKRTVAAPSVFIFPPSDEQ | |
| | | LKSGTASVVCLLNNFYPREAKVQWKVDNALQSGNSQESVTEQD | |
| | | SKDSTYSLSSTLTLSKADYEKHKVYACEVTHQGLSSPVTKSFN | |
| | | RGEC | |
| IFP-15 | | MYRMQLLSCIALSLALVTNSQVQLVQSGAEVKKPGASVKVSCK | 621 |
| with hIL-2 signal | | GSGYTFTSYWMHWVRQAPGQRLEWIGEIDPSESNTNYNQKFKG | |
| peptide | | RVTLTVDISASTAYMELSSLRSEDTAVYYCARGGYDGWDYAID | |
| | | YWGQGTLVTVSSASTKGPSVFPLAPSSKSTSGGTAALGCLVKD | |
| | | YFPEPVTVSWNSGALTSGVHTFPAVLQSSGLYSLSSVVTVPSS | |
| | | SLGTQTYICNVNHKPSNTKVDKKVEPKSCDKTHTCPPCPAPEL | |
| | | AGAPSVFLFPPKPKDTLMISRTPEVTCVVVDVSHEDPEVKFNW | |
| | | YVDGVEVHNAKTKPREEQYNSTYRVVSVLTVLHQDWLNGKEYK | |
| | | CKVSNKALPAPIEKTISKAKGQPREPQVYTLPPSRDELTKNQV | |
| | | SLTCLVKGFYPSDIAVEWESNGQPENNYKTTPPVLDSDGSFFL | |
| | | YSKLTVDKSRWQQGNVFSCSVMHEALHNHYTQKSLSLSPGKGG | |
| | | GGGGGSGGGGSGGGGSDSKCGVSEYYNKEHDICCRLCPAGSYA | |
| | | EQLCTKDNDTVCNQCPPNTFLSIPNYISSCLSCRGKCINDHVE | |
| | | DKPCTATSNRICKCKENKTCVLKTYDNSCRVCIGGGGGGGSGG | |
| | | GGSGGGGSDVVMTQSPLSLPVTPGEPASISCRSSQSLAKSYGN | |
| | | TYLSWYLQKPGQSPQLLIYGISNRFSGVPDRFSGSGSGTDFTL | |
| | | KISRVEAEDVGVYYCLQGTHQPYTFGQGTKVEIKRTVAAPSVF | |
| | | IFPPSDEQLKSGTASVVCLLNNFYPREAKVQWKVDNALQSGNS | |
| | | QESVTEQDSKDSTYSLSSTLTLSKADYEKHKVYACEVTHQGLS | |
| | | SPVTKSFNRGEC | |
| IFP-15 | | QVQLVQSGAEVKKPGASVKVSCKGSGYTFTSYWMHWVRQAPGQ | 622 |
| without hIL-2 | | RLEWIGEIDPSESNTNYNQKFKGRVTLTVDISASTAYMELSSL | |
| signal peptide | | RSEDTAVYYCARGGYDGWDYAIDYWGQGTLVTVSSASTKGPSV | |
| | | FPLAPSSKSTSGGTAALGCLVKDYFPEPVTVSWNSGALTSGVH | |
| | | TFPAVLQSSGLYSLSSVVTVPSSSLGTQTYICNVNHKPSNTKV | |
| | | DKKVEPKSCDKTHTCPPCPAPELAGAPSVFLFPPKPKDTLMIS | |
| | | RTPEVTCVVVDVSHEDPEVKFNWYVDGVEVHNAKTKPREEQYN | |
| | | STYRVVSVLTVLHQDWLNGKEYKCKVSNKALPAPIEKTISKAK | |
| | | GQPREPQVYTLPPSRDELTKNQVSLTCLVKGFYPSDIAVEWES | |
| | | NGQPENNYKTTPPVLDSDGSFFLYSKLTVDKSRWQQGNVFSCS | |
| | | VMHEALHNHYTQKSLSLSPGKGGGGGGGSGGGGSGGGGSDSKC | |
| | | GVSEYYNKEHDICCRLCPAGSYAEQLCTKDNDTVCNQCPPNTF | |
| | | LSIPNYISSCLSCRGKCINDHVEDKPCTATSNRICKCKENKTC | |
| | | VLKTYDNSCRVCIGGGGGGGSGGGGSGGGGSDVVMTQSPLSLP | |
| | | VTPGEPASISCRSSQSLAKSYGNTYLSWYLQKPGQSPQLLIYG | |
| | | ISNRFSGVPDRFSGSGSGTDFTLKISRVEAEDVGVYYCLQGTH | |
| | | QPYTFGQGTKVEIKRTVAAPSVFIFPPSDEQLKSGTASVVCLL | |
| | | NNFYPREAKVQWKVDNALQSGNSQESVTEQDSKDSTYSLSSTL | |
| | | TLSKADYEKHKVYACEVTHQGLSSPVTKSFNRGEC | |
| IFP-16 | Heavy | MYRMQLLSCIALSLALVTNSQVQLVQSGAEVKKPGASVKVSCK | 623 |
| with hIL-2 | Chain | GSGYTFTSYWMHWVRQAPGQRLEWIGEIDPSESNTNYNQKFKG | |
| signal peptide | | RVTLTVDISASTAYMELSSLRSEDTAVYYCARGGYDGWDYAID | |
| | | YWGQGTLVTVSSASTKGPSVFPLAPSSKSTSGGTAALGCLVKD | |
| | | YFPEPVTVSWNSGALTSGVHTFPAVLQSSGLYSLSSVVTVPSS | |
| | | SLGTQTYICNVNHKPSNTKVDKKVEPKSCDKTHTCPPCPAPEL | |
| | | AGAPSVFLFPPKPKDTLMISRTPEVTCVVVDVSHEDPEVKFNW | |
| | | YVDGVEVHNAKTKPREEQYNSTYRVVSVLTVLHQDWLNGKEYK | |
| | | CKVSNKALPAPIEKTISKAKGQPREPQVYTLPPSRDELTKNQV | |
| | | SLTCLVKGFYPSDIAVEWESNGQPENNYKTTPPVLDSDGSFFL | |
| | | YSKLTVDKSRWQQGNVFSCSVMHEALHNHYTQKSLSLSPGKGG | |
| | | GGGGGSGGGGSGGGGSDSKCGVSEYYNKEHDICCRLCPAGSYA | |
| | | EQLCTKDNDTVCNQCPPNTFLSIPNYISSCLSCRGKCINDHVE | |
| | | DKPCTATSNRICKCKENKTCVLKTYDNSCRVCIGGGGGGGSGG | |
| | | GGSGGGGSSTYRVRSSGLTCSTCPPGTHKERDCSLNTETICKA | |
| | | CGEGEYTAHNNSLPKCLACKSCFNATEIETKSCDPTSDTICAC | |
| | | REGYSINNLGECN | |
| | Light | DVVMTQSPLSLPVTPGEPASISCRSSQSLAKSYGNTYLSWYLQ | 592 |
| | Chain | KPGQSPQLLIYGISNRFSGVPDRFSGSGSGTDFTLKISRVEAE | |
| | | DVGVYYCLQGTHQPYTFGQGTKVEIKRTVAAPSVFIFPPSDEQ | |
| | | LKSGTASVVCLLNNFYPREAKVQWKVDNALQSGNSQESVTEQD | |
| | | SKDSTYSLSSTLTLSKADYEKHKVYACEVTHQGLSSPVTKSFN | |
| | | RGEC | |
| IFP-16 | Heavy | QVQLVQSGAEVKKPGASVKVSCKGSGYTFTSYWMHWVRQAPGQ | 624 |
| without hIL-2 | Chain | RLEWIGEIDPSESNTNYNQKFKGRVTLTVDISASTAYMELSSL | |
| signal peptide | | RSEDTAVYYCARGGYDGWDYAIDYWGQGTLVTVSSASTKGPSV | |
| | | FPLAPSSKSTSGGTAALGCLVKDYFPEPVTVSWNSGALTSGVH | |
| | | TFPAVLQSSGLYSLSSVVTVPSSSLGTQTYICNVNHKPSNTKV | |
| | | DKKVEPKSCDKTHTCPPCPAPELAGAPSVFLFPPKPKDTLMIS | |
| | | RTPEVTCVVVDVSHEDPEVKFNWYVDGVEVHNAKTKPREEQYN | |
| | | STYRVVSVLTVLHQDWLNGKEYKCKVSNKALPAPIEKTISKAK | |
| | | GQPREPQVYTLPPSRDELTKNQVSLTCLVKGFYPSDIAVEWES | |
| | | NGQPENNYKTTPPVLDSDGSFFLYSKLTVDKSRWQQGNVFSCS | |
| | | VMHEALHNHYTQKSLSLSPGKGGGGGGGSGGGGSGGGGSDSKC | |
| | | GVSEYYNKEHDICCRLCPAGSYAEQLCTKDNDTVCNQCPPNTF | |
| | | LSIPNYISSCLSCRGKCINDHVEDKPCTATSNRICKCKENKTC | |
| | | VLKTYDNSCRVCIGGGGGGGSGGGGSGGGGSSTYRVRSSGLTC | |
| | | STCPPGTHKERDCSLNTETICKACGEGEYTAHNNSLPKCLACK | |
| | | SCFNATEIETKSCDPTSDTICACREGYSINNLGECN | |
| | Light | DVVMTQSPLSLPVTPGEPASISCRSSQSLAKSYGNTYLSWYLQ | 592 |
| | Chain | KPGQSPQLLIYGISNRFSGVPDRFSGSGSGTDFTLKISRVEAE | |
| | | DVGVYYCLQGTHQPYTFGQGTKVEIKRTVAAPSVFIFPPSDEQ | |
| | | LKSGTASVVCLLNNFYPREAKVQWKVDNALQSGNSQESVTEQD | |
| | | SKDSTYSLSSTLTLSKADYEKHKVYACEVTHQGLSSPVTKSFN | |
| | | RGEC | |
| IFP-17 | Heavy | MYRMQLLSCIALSLALVTNSQVQLVQSGAEVKKPGASVKVSCK | 625 |
| with hIL-2 | Chain | GSGYTFTSYWMHWVRQAPGQRLEWIGEIDPSESNTNYNQKFKG | |
| signal peptide | | RVTLTVDISASTAYMELSSLRSEDTAVYYCARGGYDGWDYAID | |
| | | YWGQGTLVTVSSASTKGPSVFPLAPSSKSTSGGTAALGCLVKD | |
| | | YFPEPVTVSWNSGALTSGVHTFPAVLQSSGLYSLSSVVTVPSS | |
| | | SLGTQTYICNVNHKPSNTKVDKKVEPKSCDKTHTCPPCPAPEL | |
| | | AGAPSVFLFPPKPKDTLMISRTPEVTCVVVDVSHEDPEVKFNW | |
| | | YVDGVEVHNAKTKPREEQYNSTYRVVSVLTVLHQDWLNGKEYK | |
| | | CKVSNKALPAPIEKTISKAKGQPREPQVYTLPPSRDELTKNQV | |
| | | SLTCLVKGFYPSDIAVEWESNGQPENNYKTTPPVLDSDGSFFL | |
| | | YSKLTVDKSRWQQGNVFSCSVMHEALHNHYTQKSLSLSPGKGG | |
| | | GGSDSKCGVSEYYNKEHDICCRLCPAGSYAEQLCTKDNDTVCN | |
| | | QCPPNTFLSIPNYISSCLSCRGKCINDHVEDKPCTATSNRICK | |
| | | CKENKTCVLKTYDNSCRVCIGGGGSSTYRVRSSGLTCSTCPPG | |
| | | THKERDCSLNTETICKACGEGEYTAHNNSLPKCLACKSCFNAT | |
| | | EIETKSCDPTSDTICACREGYSINNLGECN | |
| | Light | DVVMTQSPLSLPVTPGEPASISCRSSQSLAKSYGNTYLSWYLQ | 592 |
| | Chain | KPGQSPQLLIYGISNRFSGVPDRFSGSGSGTDFTLKISRVEAE | |
| | | DVGVYYCLQGTHQPYTFGQGTKVEIKRTVAAPSVFIFPPSDEQ | |
| | | LKSGTASVVCLLNNFYPREAKVQWKVDNALQSGNSQESVTEQD | |
| | | SKDSTYSLSSTLTLSKADYEKHKVYACEVTHQGLSSPVTKSFN | |
| | | RGEC | |
| IFP-17 | Heavy | QVQLVQSGAEVKKPGASVKVSCKGSGYTFTSYWMHWVRQAPGQ | 626 |
| without hIL-2 | Chain | RLEWIGEIDPSESNTNYNQKFKGRVTLTVDISASTAYMELSSL | |
| signal peptide | | RSEDTAVYYCARGGYDGWDYAIDYWGQGTLVTVSSASTKGPSV | |
| | | FPLAPSSKSTSGGTAALGCLVKDYFPEPVTVSWNSGALTSGVH | |
| | | TFPAVLQSSGLYSLSSVVTVPSSSLGTQTYICNVNHKPSNTKV | |
| | | DKKVEPKSCDKTHTCPPCPAPELAGAPSVFLFPPKPKDTLMIS | |
| | | RTPEVTCVVVDVSHEDPEVKFNWYVDGVEVHNAKTKPREEQYN | |
| | | STYRVVSVLTVLHQDWLNGKEYKCKVSNKALPAPIEKTISKAK | |
| | | GQPREPQVYTLPPSRDELTKNQVSLTCLVKGFYPSDIAVEWES | |
| | | NGQPENNYKTTPPVLDSDGSFFLYSKLTVDKSRWQQGNVFSCS | |
| | | VMHEALHNHYTQKSLSLSPGKGGGGSDSKCGVSEYYNKEHDIC | |
| | | CRLCPAGSYAEQLCTKDNDTVCNQCPPNTFLSIPNYISSCLSC | |
| | | RGKCINDHVEDKPCTATSNRICKCKENKTCVLKTYDNSCRVCI | |
| | | GGGGSSTYRVRSSGLTCSTCPPGTHKERDCSLNTETICKACGE | |
| | | GEYTAHNNSLPKCLACKSCFNATEIETKSCDPTSDTICACREG | |
| | | YSINNLGECN | |
| | Light | DVVMTQSPLSLPVTPGEPASISCRSSQSLAKSYGNTYLSWYLQ | 592 |
| | Chain | KPGQSPQLLIYGISNRFSGVPDRFSGSGSGTDFTLKISRVEAE | |
| | | DVGVYYCLQGTHQPYTFGQGTKVEIKRTVAAPSVFIFPPSDEQ | |
| | | LKSGTASVVCLLNNFYPREAKVQWKVDNALQSGNSQESVTEQD | |
| | | SKDSTYSLSSTLTLSKADYEKHKVYACEVTHQGLSSPVTKSFN | |
| | | RGEC | |

In some embodiments, a fusion protein described herein comprises an amino acid sequence at least about 85%, 86%, 87%, 88%, 89%, 90%, 91%, 92%, 93%, 94%, 95%, 96%, 97%, 98%, 99%, or 100% identical to an amino acid sequence set forth in Table 16. In some embodiments, a fusion protein described herein comprises an amino acid sequence at least about 85% identical to an amino acid sequence set forth in Table 16. In some embodiments, a fusion protein described herein comprises an amino acid sequence at least about 90% identical to an amino acid sequence set forth in Table 16. In some embodiments, a fusion protein described herein comprises an amino acid sequence at least about 95% identical to an amino acid sequence set forth in Table 16. In some embodiments, a fusion protein described herein comprises an amino acid sequence at least about 99% identical to an amino acid sequence set forth in Table 16.

In some embodiments, a fusion protein described herein comprises an amino acid sequence about 85%, 86%, 87%, 88%, 89%, 90%, 91%, 92%, 93%, 94%, 95%, 96%, 97%, 98%, 99%, or 100% identical to an amino acid sequence set forth in Table 16. In some embodiments, a fusion protein described herein comprises an amino acid sequence about 85% identical to an amino acid sequence set forth in Table 16. In some embodiments, a fusion protein described herein comprises a protein ((or a functional fragment, functional variant, or functional fragment/variant thereof) comprising an amino acid sequence about 90% identical to an amino acid sequence set forth in Table 16. In some embodiments, a fusion protein described herein comprises an amino acid sequence about 95% identical to an amino acid sequence set forth in Table 16. In some embodiments, a fusion protein described herein comprises an amino acid sequence about 99% identical to an amino acid sequence set forth in Table 16. In some embodiments, a fusion protein described herein comprises an amino acid sequence about 100% identical to an amino acid sequence set forth in Table 16.

In some embodiments, a fusion protein described herein comprises an amino acid sequence set forth in Table 16, and further comprising 1 or more but less than 15% (less than 12%, less than 10%, less than 8%), amino acid variations (e.g., substitutions, additions, deletions, etc.). In some embodiments, a fusion protein described herein comprises an amino acid sequence set forth in Table 16, and further comprising at least about 1, 2, 3, 4, 5, 6, 7, 8, 9, or 10 amino acid variations (e.g., substitutions, additions, deletions, etc.). In some embodiments, a fusion protein described herein comprises an amino acid sequence set forth in Table 16, and further consisting of at least about 1, 2, 3, 4, 5, 6, 7, 8, 9, or 10 amino acid variations (e.g., substitutions, additions, deletions, etc.). In some embodiments, a fusion protein described herein comprises an amino acid sequence set forth in Table 16, and further comprising about 1, 2, 3, 4, 5, 6, 7, 8, 9, or 10 amino acid variations (e.g., substitutions, additions, deletions, etc.). In some embodiments, a fusion protein described herein comprises an amino acid sequence set forth in Table 16, and further consisting of about 1, 2, 3, 4, 5, 6, 7, 8, 9, or 10 amino acid variations (e.g., substitutions, additions, deletions, etc.). In some embodiments, a fusion protein described herein comprises an amino acid sequence set forth in Table 16, and further comprising or no more than about 1, 2, 3, 4, 5, 6, 7, 8, 9, or 10 amino acid variations (e.g., substitutions, additions, deletions, etc.).

In some embodiments, a fusion protein described herein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, a fusion protein described herein comprises an amino acid sequence at least 85% identical to the amino acid sequence set forth in any one of SEQ ID NOS: 593-612. In some embodiments, a fusion protein described herein comprises an amino acid sequence at least 90% identical to the amino acid sequence set forth in any one of SEQ ID NOS: 593-612. In some embodiments, a fusion protein described herein comprises an amino acid sequence at least 95% identical to the amino acid sequence set forth in any one of SEQ ID NOS: 593-612. In some embodiments, a fusion protein described herein comprises an amino acid sequence at least 99% identical to the amino acid sequence set forth in any one of SEQ ID NOS: 593-612.

In some embodiments, a fusion protein described herein comprises an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, a fusion protein described herein comprises an amino acid sequence 85% identical to the amino acid sequence set forth in any one of SEQ ID NOS: 593-612. In some embodiments, a fusion protein described herein comprises an amino acid sequence 90% identical to the amino acid sequence set forth in any one of SEQ ID NOS: 593-612. In some embodiments, a fusion protein described herein comprises an amino acid sequence 95% identical to the amino acid sequence set forth in any one of SEQ ID NOS: 593-612. In some embodiments, a fusion protein described herein comprises an amino acid sequence 99% identical to the amino acid sequence set forth in any one of SEQ ID NOS: 593-612. In some embodiments, a fusion protein described herein comprises an amino acid sequence 100% identical to the amino acid sequence set forth in any one of SEQ ID NOS: 593-612.

In some embodiments, a fusion protein described herein comprises the amino acid sequence set forth in any one of SEQ ID NOS: 593-612, and further comprising 1 or more but less than 15% (less than 12%, less than 10%, less than 8%), amino acid variations (e.g., substitutions, additions, deletions, etc.). In some embodiments, a fusion protein described herein comprises the amino acid sequence set forth in any one of SEQ ID NOS: 593-612, and further comprising at least about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 593-612, and further comprising about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 593-612, and further consisting of about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 593-612, and further comprising or consists of no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 16. In some embodiments, a fusion protein described herein comprises a protein (or a functional fragment, functional variant, or functional fragment/variant thereof) comprising an amino acid sequence at least about 85% identical to the amino acid sequence of a protein set forth in Table 16. In some embodiments, a fusion protein described herein comprises a protein (or a functional fragment, functional variant, or functional fragment/variant thereof) comprising an amino acid sequence at least about 90% identical to the amino acid sequence of a protein set forth in Table 16. In some embodiments, a fusion protein described herein comprises a protein (or a functional fragment, functional variant, or functional fragment/variant thereof) comprising an amino acid sequence at least about 95% identical to the amino acid sequence of a protein set forth in Table 16. In some embodiments, a fusion protein described herein comprises a protein (or a functional fragment, functional variant, or functional fragment/variant thereof) comprising an amino acid sequence at least about 99% identical to the amino acid sequence of a protein set forth in Table 16.

In some embodiments, a fusion protein described herein comprises a protein (or a functional fragment, functional variant, or functional fragment/variant thereof) comprising an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 16. In some embodiments, a fusion protein described herein comprises a protein (or a functional fragment, functional variant, or functional fragment/variant thereof) comprising an amino acid sequence about 85% identical to the amino acid sequence of a protein set forth in Table 16. In some embodiments, a fusion protein described herein comprises a protein ((or a functional fragment, functional variant, or functional fragment/variant thereof) comprising an amino acid sequence about 90% identical to the amino acid sequence of a protein set forth in Table 16. In some embodiments, a fusion protein described herein comprises a protein (or a functional fragment, functional variant, or functional fragment/variant thereof) comprising an amino acid sequence about 95% identical to the amino acid sequence of a protein set forth in Table 16. In some embodiments, a fusion protein described herein comprises a protein (or a functional fragment, functional variant, or functional fragment/variant thereof) comprising an amino acid sequence about 99% identical to the amino acid sequence of a protein set forth in Table 16. In some embodiments, a fusion protein described herein comprises a protein (or a functional fragment, functional variant, or functional fragment/variant thereof) comprising an amino acid sequence about 100% identical to the amino acid sequence of a protein set forth in Table 16.

In some embodiments, a fusion protein described herein comprises a protein (or a functional fragment, functional variant, or functional fragment/variant thereof) consisting of an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 16. In some embodiments, a fusion protein described herein comprises a protein (or a functional fragment, functional variant, or functional fragment/variant thereof) consisting of an amino acid sequence at least about 85% identical to the amino acid sequence of a protein set forth in Table 16. In some embodiments, a fusion protein described herein comprises a protein (or a functional fragment, functional variant, or functional fragment/variant thereof) consisting of an amino acid sequence at least about 90% identical to the amino acid sequence of a protein set forth in Table 16. In some embodiments, a fusion protein described herein comprises a protein (or a functional fragment, functional variant, or functional fragment/variant thereof) consisting of an amino acid sequence at least about 95% identical to the amino acid sequence of a protein set forth in Table 16. In some embodiments, a fusion protein described herein comprises a protein (or a functional fragment, functional variant, or functional fragment/variant thereof) consisting of an amino acid sequence at least about 99% identical to the amino acid sequence of a protein set forth in Table 16.

In some embodiments, a fusion protein described herein comprises a protein (or a functional fragment, functional variant, or functional fragment/variant thereof) consisting of an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 16. In some embodiments, a fusion protein described herein comprises a protein (or a functional fragment, functional variant, or functional fragment/variant thereof) consisting of an amino acid sequence about 85% identical to the amino acid sequence of a protein set forth in Table 16. In some embodiments, a fusion protein described herein comprises a protein (or a functional fragment, functional variant, or functional fragment/variant thereof) consisting of an amino acid sequence about 90% identical to the amino acid sequence of a protein set forth in Table 16. In some embodiments, a fusion protein described herein comprises a protein (or a functional fragment, functional variant, or functional fragment/variant thereof) consisting of an amino acid sequence about 95% identical to the amino acid sequence of a protein set forth in Table 16. In some embodiments, a fusion protein described herein comprises a protein (or a functional fragment, functional variant, or functional fragment/variant thereof) consisting of an amino acid sequence about 99% identical to the amino acid sequence of a protein set forth in Table 16. In some embodiments, a fusion protein described herein comprises a protein (or a functional fragment, functional variant, or functional fragment/variant thereof) consisting of an amino acid sequence about 100% identical to the amino acid sequence of a protein set forth in Table 16.

In some embodiments, a fusion protein described herein comprises a protein (or a functional fragment, functional variant, or functional fragment/variant thereof) comprising the amino acid sequence of a protein set forth in Table 16, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 16, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 16, and further consisting of at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 16, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 16, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence of a protein set forth in Table 16, and further comprising or no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence of a protein set forth in Table 16, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 16, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 16, and further consisting of at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 16, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 16, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence of a protein set forth in Table 16, and further comprising or no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence at least 85% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence at least 90% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence at least 95% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence at least 99% identical to the amino acid sequence set forth in any one of SEQ ID NOS: 593-612.

In some embodiments, a fusion protein described herein comprises a protein (or a functional fragment, functional variant, or functional fragment/variant thereof) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence 85% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence 90% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence 95% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence 99% identical to the amino acid sequence set forth in any one of SEQ ID NOS: 593-612. In some embodiments, a fusion protein described herein comprises a protein (or a functional fragment, functional variant, or functional fragment/variant thereof) comprising an amino acid sequence 100% identical to the amino acid sequence set forth in any one of SEQ ID NOS: 593-612.

In some embodiments, a fusion protein described herein comprises a protein (or a functional fragment, functional variant, or functional fragment/variant thereof) consisting of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence at least 85% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence at least 90% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence at least 95% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence at least 99% identical to the amino acid sequence set forth in any one of SEQ ID NOS: 593-612.

In some embodiments, a fusion protein described herein comprises a protein (or a functional fragment, functional variant, or functional fragment/variant thereof) consisting of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence 85% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence 90% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence 95% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence 99% identical to the amino acid sequence set forth in any one of SEQ ID NOS: 593-612. In some embodiments, a fusion protein described herein comprises a protein (or a functional fragment, functional variant, or functional fragment/variant thereof) consisting of an amino acid sequence 100% identical to the amino acid sequence set forth in any one of SEQ ID NOS: 593-612.

In some embodiments, a fusion protein described herein comprises a protein (or a functional fragment, functional variant, or functional fragment/variant thereof) comprising the amino acid sequence set forth in any one of SEQ ID NOS: 593-612, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 593-612, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 593-612, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 593-612, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) comprising the amino acid sequence set forth in any one of SEQ ID NOS: 593-612, and further comprising or consists of no more than about 1, 2, 3, 4, 5, 6, 7, 8, 9, or 10 amino acid variations (e.g., substitutions, additions, deletions, etc.).

In some embodiments, a fusion protein described herein comprises a protein (or a functional fragment, functional variant, or functional fragment/variant thereof) consisting of the amino acid sequence set forth in any one of SEQ ID NOS: 593-612, and further comprising 1 or more but less than 15% (less than 12%, less than 10%, less than 8%), amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 593-612, and further comprising at least about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 593-612, and further comprising about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 593-612, and further consisting of about 1, 2, 3, 4, 5, 6, 7, 8, 9, or 10 amino acid variations (e.g., substitutions, additions, deletions, etc.). In some embodiments, a fusion protein described herein comprises a protein (or a functional fragment, functional variant, or functional fragment/variant thereof) consisting of the amino acid sequence set forth in any one of SEQ ID NOS: 593-612, and further consisting of no more than about 1, 2, 3, 4, 5, 6, 7, 8, 9, or 10 amino acid variations (e.g., substitutions, additions, deletions, etc.).

In some embodiments, the fusion protein is a homodimeric fusion protein comprising two polypeptide chains each comprising an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 16. In some embodiments, the fusion protein is a homodimeric fusion protein comprising two polypeptide chains each comprising an amino acid sequence at least about 85% identical to the amino acid sequence of a protein set forth in Table 16. In some embodiments, the fusion protein is a homodimeric fusion protein comprising two polypeptide chains each comprising an amino acid sequence at least about 90% identical to the amino acid sequence of a protein set forth in Table 16. In some embodiments, the fusion protein is a homodimeric fusion protein comprising two polypeptide chains each comprising an amino acid sequence at least about 95% identical to the amino acid sequence of a protein set forth in Table 16. In some embodiments, the fusion protein is a homodimeric fusion protein comprising two polypeptide chains each comprising an amino acid sequence at least about 99% identical to the amino acid sequence of a protein set forth in Table 16.

In some embodiments, the fusion protein is a homodimeric fusion protein comprising two polypeptide chains each comprising an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 16. In some embodiments, the fusion protein is a homodimeric fusion protein comprising two polypeptide chains each comprising an amino acid sequence about 85% identical to the amino acid sequence of a protein set forth in Table 16. In some embodiments, a fusion protein described herein comprises a protein ((or a functional fragment, functional variant, or functional fragment/variant thereof) comprising an amino acid sequence about 90% identical to the amino acid sequence of a protein set forth in Table 16. In some embodiments, the fusion protein is a homodimeric fusion protein comprising two polypeptide chains each comprising an amino acid sequence about 95% identical to the amino acid sequence of a protein set forth in Table 16. In some embodiments, the fusion protein is a homodimeric fusion protein comprising two polypeptide chains each comprising an amino acid sequence about 99% identical to the amino acid sequence of a protein set forth in Table 16. In some embodiments, the fusion protein is a homodimeric fusion protein comprising two polypeptide chains each comprising an amino acid sequence about 100% identical to the amino acid sequence of a protein set forth in Table 16.

In some embodiments, the fusion protein is a homodimeric fusion protein comprising two polypeptide chains each consisting of an amino acid sequence at least about 85%, 86%, 87%, 88%, 89%, 90%, 91%, 92%, 93%, 94%, 95%, 96%, 97%, 98%, 99%, or 100% identical to the amino acid sequence of a protein set forth in Table 16. In some embodiments, the fusion protein is a homodimeric fusion protein comprising two polypeptide chains each consisting of an amino acid sequence at least about 85% identical to the amino acid sequence of a protein set forth in Table 16. In some embodiments, the fusion protein is a homodimeric fusion protein comprising two polypeptide chains each consisting of an amino acid sequence at least about 90% identical to the amino acid sequence of a protein set forth in Table 16. In some embodiments, the fusion protein is a homodimeric fusion protein comprising two polypeptide chains each consisting of an amino acid sequence at least about 95% identical to the amino acid sequence of a protein set forth in Table 16. In some embodiments, the fusion protein is a homodimeric fusion protein comprising two polypeptide chains each consisting of an amino acid sequence at least about 99% identical to the amino acid sequence of a protein set forth in Table 16.

In some embodiments, the fusion protein is a homodimeric fusion protein comprising two polypeptide chains each consisting of an amino acid sequence about 85%, 86%, 87%, 88%, 89%, 90%, 91%, 92%, 93%, 94%, 95%, 96%, 97%, 98%, 99%, or 100% identical to the amino acid sequence of a protein set forth in Table 16. In some embodiments, the fusion protein is a homodimeric fusion protein comprising two polypeptide chains each consisting of an amino acid sequence about 85% identical to the amino acid sequence of a protein set forth in Table 16. In some embodiments, the fusion protein is a homodimeric fusion protein comprising two polypeptide chains each consisting of an amino acid sequence about 90% identical to the amino acid sequence of a protein set forth in Table 16. In some embodiments, the fusion protein is a homodimeric fusion protein comprising two polypeptide chains each consisting of an amino acid sequence about 95% identical to the amino acid sequence of a protein set forth in Table 16. In some embodiments, the fusion protein is a homodimeric fusion protein comprising two polypeptide chains each consisting of an amino acid sequence about 99% identical to the amino acid sequence of a protein set forth in Table 16. In some embodiments, the fusion protein is a homodimeric fusion protein comprising two polypeptide chains each consisting of an amino acid sequence about 100% identical to the amino acid sequence of a protein set forth in Table 16.

In some embodiments, the fusion protein is a homodimeric fusion protein comprising two polypeptide chains each comprising the amino acid sequence of a protein set forth in Table 16, and further comprising 1 or more but less than 15% (less than 12%, less than 10%, less than 8%), amino acid variations (e.g., substitutions, additions, deletions, etc.). In some embodiments, the fusion protein is a homodimeric fusion protein comprising two polypeptide chains each comprising the amino acid sequence of a protein set forth in Table 16, and further comprising at least about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each comprising the amino acid sequence of a protein set forth in Table 16, and further consisting of at least about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each comprising the amino acid sequence of a protein set forth in Table 16, and further comprising about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each comprising the amino acid sequence of a protein set forth in Table 16, and further consisting of about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each comprising the amino acid sequence of a protein set forth in Table 16, and further comprising or no more than about 1, 2, 3, 4, 5, 6, 7, 8, 9, or 10 amino acid variations (e.g., substitutions, additions, deletions, etc.).

In some embodiments, the fusion protein is a homodimeric fusion protein comprising two polypeptide chains each consisting of the amino acid sequence of a protein set forth in Table 16, and further comprising 1 or more but less than 15% (less than 12%, less than 10%, less than 8%), amino acid variations (e.g., substitutions, additions, deletions, etc.). In some embodiments, the fusion protein is a homodimeric fusion protein comprising two polypeptide chains each consisting of the amino acid sequence of a protein set forth in Table 16, and further comprising at least about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each consisting of the amino acid sequence of a protein set forth in Table 16, and further consisting of at least about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each consisting of the amino acid sequence of a protein set forth in Table 16, and further comprising about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each consisting of the amino acid sequence of a protein set forth in Table 16, and further consisting of about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each consisting of the amino acid sequence of a protein set forth in Table 16, and further comprising or no more than about 1, 2, 3, 4, 5, 6, 7, 8, 9, or 10 amino acid variations (e.g., substitutions, additions, deletions, etc.).

In some embodiments, the fusion protein is a homodimeric fusion protein comprising two polypeptide chains each comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, the fusion protein is a homodimeric fusion protein comprising two polypeptide chains each comprising an amino acid sequence at least 85% identical to the amino acid sequence set forth in any one of SEQ ID NOS: 593-612. In some embodiments, the fusion protein is a homodimeric fusion protein comprising two polypeptide chains each comprising an amino acid sequence at least 90% identical to the amino acid sequence set forth in any one of SEQ ID NOS: 593-612. In some embodiments, the fusion protein is a homodimeric fusion protein comprising two polypeptide chains each comprising an amino acid sequence at least 95% identical to the amino acid sequence set forth in any one of SEQ ID NOS: 593-612. In some embodiments, the fusion protein is a homodimeric fusion protein comprising two polypeptide chains each comprising an amino acid sequence at least 99% identical to the amino acid sequence set forth in any one of SEQ ID NOS: 593-612.

In some embodiments, the fusion protein is a homodimeric fusion protein comprising two polypeptide chains each comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, the fusion protein is a homodimeric fusion protein comprising two polypeptide chains each comprising an amino acid sequence 85% identical to the amino acid sequence set forth in any one of SEQ ID NOS: 593-612. In some embodiments, the fusion protein is a homodimeric fusion protein comprising two polypeptide chains each comprising an amino acid sequence 90% identical to the amino acid sequence set forth in any one of SEQ ID NOS: 593-612. In some embodiments, the fusion protein is a homodimeric fusion protein comprising two polypeptide chains each comprising an amino acid sequence 95% identical to the amino acid sequence set forth in any one of SEQ ID NOS: 593-612. In some embodiments, the fusion protein is a homodimeric fusion protein comprising two polypeptide chains each comprising an amino acid sequence 99% identical to the amino acid sequence set forth in any one of SEQ ID NOS: 593-612. In some embodiments, the fusion protein is a homodimeric fusion protein comprising two polypeptide chains each comprising an amino acid sequence 100% identical to the amino acid sequence set forth in any one of SEQ ID NOS: 593-612.

In some embodiments, the fusion protein is a homodimeric fusion protein comprising two polypeptide chains each consisting of an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, the fusion protein is a homodimeric fusion protein comprising two polypeptide chains each consisting of an amino acid sequence at least 85% identical to the amino acid sequence set forth in any one of SEQ ID NOS: 593-612. In some embodiments, the fusion protein is a homodimeric fusion protein comprising two polypeptide chains each consisting of an amino acid sequence at least 90% identical to the amino acid sequence set forth in any one of SEQ ID NOS: 593-612. In some embodiments, the fusion protein is a homodimeric fusion protein comprising two polypeptide chains each consisting of an amino acid sequence at least 95% identical to the amino acid sequence set forth in any one of SEQ ID NOS: 593-612. In some embodiments, the fusion protein is a homodimeric fusion protein comprising two polypeptide chains each consisting of an amino acid sequence at least 99% identical to the amino acid sequence set forth in any one of SEQ ID NOS: 593-612.

In some embodiments, the fusion protein is a homodimeric fusion protein comprising two polypeptide chains each consisting of an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 593-612. In some embodiments, the fusion protein is a homodimeric fusion protein comprising two polypeptide chains each consisting of an amino acid sequence 85% identical to the amino acid sequence set forth in any one of SEQ ID NOS: 593-612. In some embodiments, the fusion protein is a homodimeric fusion protein comprising two polypeptide chains each consisting of an amino acid sequence 90% identical to the amino acid sequence set forth in any one of SEQ ID NOS: 593-612. In some embodiments, the fusion protein is a homodimeric fusion protein comprising two polypeptide chains each consisting of an amino acid sequence 95% identical to the amino acid sequence set forth in any one of SEQ ID NOS: 593-612. In some embodiments, the fusion protein is a homodimeric fusion protein comprising two polypeptide chains each consisting of an amino acid sequence 99% identical to the amino acid sequence set forth in any one of SEQ ID NOS: 593-612. In some embodiments, the fusion protein is a homodimeric fusion protein comprising two polypeptide chains each consisting of an amino acid sequence 100% identical to the amino acid sequence set forth in any one of SEQ ID NOS: 593-612.

In some embodiments, the fusion protein is a homodimeric fusion protein comprising two polypeptide chains each comprising the amino acid sequence set forth in any one of SEQ ID NOS: 593-612, and further comprising 1 or more but less than 15% (less than 12%, less than 10%, less than 8%), amino acid variations (e.g., substitutions, additions, deletions, etc.). In some embodiments, the fusion protein is a homodimeric fusion protein comprising two polypeptide chains each comprising the amino acid sequence set forth in any one of SEQ ID NOS: 593-612, and further comprising at least about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each comprising the amino acid sequence set forth in any one of SEQ ID NOS: 593-612, and further comprising about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each comprising the amino acid sequence set forth in any one of SEQ ID NOS: 593-612, and further consisting of about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each comprising the amino acid sequence set forth in any one of SEQ ID NOS: 593-612, and further comprising or consists of no more than about 1, 2, 3, 4, 5, 6, 7, 8, 9, or 10 amino acid variations (e.g., substitutions, additions, deletions, etc.).

In some embodiments, the fusion protein is a homodimeric fusion protein comprising two polypeptide chains each consisting of the amino acid sequence set forth in any one of SEQ ID NOS: 593-612, and further comprising 1 or more but less than 15% (less than 12%, less than 10%, less than 8%), amino acid variations (e.g., substitutions, additions, deletions, etc.). In some embodiments, the fusion protein is a homodimeric fusion protein comprising two polypeptide chains each consisting of the amino acid sequence set forth in any one of SEQ ID NOS: 593-612, and further comprising at least about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each consisting of the amino acid sequence set forth in any one of SEQ ID NOS: 593-612, and further comprising about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each consisting of the amino acid sequence set forth in any one of SEQ ID NOS: 593-612, and further consisting of about 1, 2, 3, 4, 5, 6, 7, 8, 9, or 10 amino acid variations (e.g., substitutions, additions, deletions, etc.). In some embodiments, the fusion protein is a homodimeric fusion protein comprising two polypeptide chains each consisting of the amino acid sequence set forth in any one of SEQ ID NOS: 593-612, and further consisting of no more than about 1, 2, 3, 4, 5, 6, 7, 8, 9, or 10 amino acid variations (e.g., substitutions, additions, deletions, etc.). In some embodiments, a fusion protein described herein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 592, 613-626. In some embodiments, a fusion protein described herein comprises an amino acid sequence at least 85% identical to the amino acid sequence set forth in any one of SEQ ID NOS: 592, 613-626. In some embodiments, a fusion protein described herein comprises an amino acid sequence at least 90% identical to the amino acid sequence set forth in any one of SEQ ID NOS: 592, 613-626. In some embodiments, a fusion protein described herein comprises an amino acid sequence at least 95% identical to the amino acid sequence set forth in any one of SEQ ID NOS: 592, 613-626. In some embodiments, a fusion protein described herein comprises an amino acid sequence at least 99% identical to the amino acid sequence set forth in any one of SEQ ID NOS: 592, 613-626.

In some embodiments, a fusion protein described herein comprises an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 592, 613-626. In some embodiments, a fusion protein described herein comprises an amino acid sequence 85% identical to the amino acid sequence set forth in any one of SEQ ID NOS: 592, 613-626. In some embodiments, a fusion protein described herein comprises an amino acid sequence 90% identical to the amino acid sequence set forth in any one of SEQ ID NOS: 592, 613-626. In some embodiments, a fusion protein described herein comprises an amino acid sequence 95% identical to the amino acid sequence set forth in any one of SEQ ID NOS: 592, 613-626. In some embodiments, a fusion protein described herein comprises an amino acid sequence 99% identical to the amino acid sequence set forth in any one of SEQ ID NOS: 592, 613-626. In some embodiments, a fusion protein described herein comprises an amino acid sequence 100% identical to the amino acid sequence set forth in any one of SEQ ID NOS: 592, 613-626.

In some embodiments, a fusion protein described herein comprises the amino acid sequence set forth in any one of SEQ ID NOS: 592, 613-626, and further comprising 1 or more but less than 15% (less than 12%, less than 10%, less than 8%), amino acid variations (e.g., substitutions, additions, deletions, etc.). In some embodiments, a fusion protein described herein comprises the amino acid sequence set forth in any one of SEQ ID NOS: 592, 613-626, and further comprising at least about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 592, 613-626, and further comprising about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 592, 613-626, and further consisting of about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 592, 613-626, and further comprising or consists of no more than about 1, 2, 3, 4, 5, 6, 7, 8, 9, or 10 amino acid variations (e.g., substitutions, additions, deletions, etc.).

In some embodiments, a fusion protein described herein comprises an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 613-616 or 621-622. In some embodiments, a fusion protein described herein comprises an amino acid sequence at least 85% identical to the amino acid sequence set forth in any one of SEQ ID NOS: 613-616 or 621-622. In some embodiments, a fusion protein described herein comprises an amino acid sequence at least 90% identical to the amino acid sequence set forth in any one of SEQ ID NOS: 613-616 or 621-622. In some embodiments, a fusion protein described herein comprises an amino acid sequence at least 95% identical to the amino acid sequence set forth in any one of SEQ ID NOS: 613-616 or 621-622. In some embodiments, a fusion protein described herein comprises an amino acid sequence at least 99% identical to the amino acid sequence set forth in any one of SEQ ID NOS: 613-616 or 621-622.

In some embodiments, a fusion protein described herein comprises an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 613-616 or 621-622. In some embodiments, a fusion protein described herein comprises an amino acid sequence 85% identical to the amino acid sequence set forth in any one of SEQ ID NOS: 613-616 or 621-622. In some embodiments, a fusion protein described herein comprises an amino acid sequence 90% identical to the amino acid sequence set forth in any one of SEQ ID NOS: 613-616 or 621-622. In some embodiments, a fusion protein described herein comprises an amino acid sequence 95% identical to the amino acid sequence set forth in any one of SEQ ID NOS: 613-616 or 621-622. In some embodiments, a fusion protein described herein comprises an amino acid sequence 99% identical to the amino acid sequence set forth in any one of SEQ ID NOS: 613-616 or 621-622. In some embodiments, a fusion protein described herein comprises an amino acid sequence 100% identical to the amino acid sequence set forth in any one of SEQ ID NOS: 613-616 or 621-622.

In some embodiments, a fusion protein described herein comprises the amino acid sequence set forth in any one of SEQ ID NOS: 613-616 or 621-622, and further comprising 1 or more but less than 15% (less than 12%, less than 10%, less than 8%), amino acid variations (e.g., substitutions, additions, deletions, etc.). In some embodiments, a fusion protein described herein comprises the amino acid sequence set forth in any one of SEQ ID NOS: 613-616 or 621-622, and further comprising at least about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 613-616 or 621-622, and further comprising about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 613-616 or 621-622, and further consisting of about 1, 2, 3, 4, 5, 6, 7, 8, 9, or 10 amino acid variations (e.g., substitutions, additions, deletions, etc.). In some embodiments, a fusion protein described herein comprises the amino acid sequence set forth in any one of SEQ ID NOS: 613-616 or 621-622, and further comprising or consists of no more than about 1, 2, 3, 4, 5, 6, 7, 8, 9, or 10 amino acid variations (e.g., substitutions, additions, deletions, etc.).

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 617-620 or 623-626; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 617-620 or 623-626.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in any one of SEQ ID NOS: 617-620 or 623-626; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in any one of SEQ ID NOS: 617-620 or 623-626. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in any one of SEQ ID NOS: 617-620 or 623-626; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in any one of SEQ ID NOS: 617-620 or 623-626.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in any one of SEQ ID NOS: 617-620 or 623-626; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in any one of SEQ ID NOS: 617-620 or 623-626. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in any one of SEQ ID NOS: 617-620 or 623-626; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in any one of SEQ ID NOS: 617-620 or 623-626.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 617-620 or 623-626; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 617-620 or 623-626.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in any one of SEQ ID NOS: 617-620 or 623-626; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in any one of SEQ ID NOS: 617-620 or 623-626.

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 617; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 617.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in SEQ ID NO: 617; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in SEQ ID NO: 617. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in SEQ ID NO: 617; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in SEQ ID NO: 617.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in SEQ ID NO: 617; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in SEQ ID NO: 617. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in SEQ ID NO: 617; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in SEQ ID NO: 617.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 617; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 617.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in SEQ ID NO: 617; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in SEQ ID NO: 617.

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 618; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 618.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in SEQ ID NO: 618; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in SEQ ID NO: 618. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in SEQ ID NO: 618; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in SEQ ID NO: 618.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in SEQ ID NO: 618; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in SEQ ID NO: 618. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in SEQ ID NO: 618; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in SEQ ID NO: 618.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 618; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 618.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in SEQ ID NO: 618; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in SEQ ID NO: 618.

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 619; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 619.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in SEQ ID NO: 619; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in SEQ ID NO: 619. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in SEQ ID NO: 619; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in SEQ ID NO: 619.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in SEQ ID NO: 619; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in SEQ ID NO: 619. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in SEQ ID NO: 619; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in SEQ ID NO: 619.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 619; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 619.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in SEQ ID NO: 619; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in SEQ ID NO: 619.

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 620; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 620.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in SEQ ID NO: 620; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in SEQ ID NO: 620. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in SEQ ID NO: 620; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in SEQ ID NO: 620.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in SEQ ID NO: 620; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in SEQ ID NO: 620. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in SEQ ID NO: 620; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in SEQ ID NO: 620.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 620; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 620.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in SEQ ID NO: 620; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in SEQ ID NO: 620.

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 623; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 623.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in SEQ ID NO: 623; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in SEQ ID NO: 623. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in SEQ ID NO: 623; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in SEQ ID NO: 623.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in SEQ ID NO: 623; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in SEQ ID NO: 623. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in SEQ ID NO: 623; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in SEQ ID NO: 623.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 623; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 623.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in SEQ ID NO: 623; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in SEQ ID NO: 623.

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 624; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 624.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in SEQ ID NO: 624; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in SEQ ID NO: 624. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in SEQ ID NO: 624; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in SEQ ID NO: 624.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in SEQ ID NO: 624; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in SEQ ID NO: 624. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in SEQ ID NO: 624; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in SEQ ID NO: 624.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 624; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 624.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in SEQ ID NO: 624; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in SEQ ID NO: 624.

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 625; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 625.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in SEQ ID NO: 625; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in SEQ ID NO: 625. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in SEQ ID NO: 625; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in SEQ ID NO: 625.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in SEQ ID NO: 625; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in SEQ ID NO: 625. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in SEQ ID NO: 625; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in SEQ ID NO: 625.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 625; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 625.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in SEQ ID NO: 625; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in SEQ ID NO: 625.

In some embodiments, a fusion protein described herein comprises a first polypeptide (e.g., a first light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 626; a third polypeptide (e.g., a second light chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence at least 85%, 86%, 87%, 88%, 89%, 90%, 91%, 92%, 93%, 94%, 95%, 96%, 97%, 98%, 99%, or 100% identical to the amino acid sequence set forth in SEQ ID NO: 626.

In some embodiments, a fusion protein described herein comprises a first polypeptide (e.g., a first light chain) comprising an amino acid sequence at least 85% identical to the amino acid sequence set forth in SEQ ID NO: 592; a second polypeptide (e.g., a first heavy chain) comprising an amino acid sequence at least 85% identical to the amino acid sequence set forth in SEQ ID NO: 626; a third polypeptide (e.g., a second light chain) comprising an amino acid sequence at least 85% identical to the amino acid sequence set forth in SEQ ID NO: 592; and a fourth polypeptide (e.g., a second heavy chain) comprising an amino acid sequence at least 85% identical to the amino acid sequence set forth in SEQ ID NO: 626. In some embodiments, a fusion protein described herein comprises a first polypeptide (e.g., a first light chain) comprising an amino acid sequence at least 90% identical to the amino acid sequence set forth in SEQ ID NO: 592; a second polypeptide (e.g., a first heavy chain) comprising an amino acid sequence at least 90% identical to the amino acid sequence set forth in SEQ ID NO: 626; a third polypeptide (e.g., a second light chain) comprising an amino acid sequence at least 90% identical to the amino acid sequence set forth in SEQ ID NO: 592; and a fourth polypeptide (e.g., a second heavy chain) comprising an amino acid sequence at least 90% identical to the amino acid sequence set forth in SEQ ID NO: 626.

In some embodiments, a fusion protein described herein comprises a first polypeptide (e.g., a first light chain) comprising an amino acid sequence at least 95% identical to the amino acid sequence set forth in SEQ ID NO: 592; a second polypeptide (e.g., a first heavy chain) comprising an amino acid sequence at least 95% identical to the amino acid sequence set forth in SEQ ID NO: 626; a third polypeptide (e.g., a second light chain) comprising an amino acid sequence at least 95% identical to the amino acid sequence set forth in SEQ ID NO: 592; and a fourth polypeptide (e.g., a second heavy chain) comprising an amino acid sequence at least 95% identical to the amino acid sequence set forth in SEQ ID NO: 626. In some embodiments, a fusion protein described herein comprises a first polypeptide (e.g., a first light chain) comprising an amino acid sequence at least 99% identical to the amino acid sequence set forth in SEQ ID NO: 592; a second polypeptide (e.g., a first heavy chain) comprising an amino acid sequence at least 99% identical to the amino acid sequence set forth in SEQ ID NO: 626; a third polypeptide (e.g., a second light chain) comprising an amino acid sequence at least 99% identical to the amino acid sequence set forth in SEQ ID NO: 592; and a fourth polypeptide (e.g., a second heavy chain) comprising an amino acid sequence at least 99% identical to the amino acid sequence set forth in SEQ ID NO: 626.

In some embodiments, a fusion protein described herein comprises a first polypeptide (e.g., a first light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; a second polypeptide (e.g., a first heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 626; a third polypeptide (e.g., a second light chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 592; and a fourth polypeptide (e.g., a second heavy chain) comprising an amino acid sequence 85%, 86%, 87%, 88%, 89%, 90%, 91%, 92%, 93%, 94%, 95%, 96%, 97%, 98%, 99%, or 100% identical to the amino acid sequence set forth in SEQ ID NO: 626.

In some embodiments, a fusion protein described herein comprises a first polypeptide (e.g., a first light chain) comprising an amino acid sequence 100% identical to the amino acid sequence set forth in SEQ ID NO: 592; a second polypeptide (e.g., a first heavy chain) comprising an amino acid sequence 100% identical to the amino acid sequence set forth in SEQ ID NO: 626; a third polypeptide (e.g., a second light chain) comprising an amino acid sequence 100% identical to the amino acid sequence set forth in SEQ ID NO: 592; and a fourth polypeptide (e.g., a second heavy chain) comprising an amino acid sequence 100% identical to the amino acid sequence set forth in SEQ ID NO: 626.

### 5.9 Exemplary Properties of Immunomodulatory Fusion Proteins

The immunomodulatory fusion proteins described herein can exhibit a number of structural and functional properties.

For example, in some embodiments, the amino acid sequence of an immunomodulatory fusion protein described herein contains no more than 3000, 2000, 1500, 1000, 500, 400, 300, 200, or 100 total amino acids (e.g., accounting for all polypeptide chains of a fusion protein). In some embodiments, the amino acid sequence of an immunomodulatory fusion protein described herein contains is less than 3000, 2000, 1500, 1000, 500, 400, 300, 200, or 100 total amino acids (e.g., accounting for all polypeptide chains of a fusion protein). In some embodiments, the amino acid sequence of an immunomodulatory fusion protein described herein contains from about 100-3000, 100-2000, 100-1500, 100-1000, 100-500, 100-400, 100-300, 100-200, 200-3000, 200-2000, 200-1500, 200-1000, 200-500, 200-400,200-300, 300-3000, 300-2000, 300-1500, 300-1000, 300-500, 300-400, 400-3000, 400-2000, 400-1500, 400-1000, 400-500, 500-3000, 500-2000, 500-1500, 500-1000, 1000-3000, 1000-2000, or 1000-1500 total amino acids (e.g., accounting for all polypeptide chains of a fusion protein).

In some embodiments, the immunomodulatory fusion protein is immunosuppressive (e.g., when administered to a subject). In some embodiments, the immunomodulatory fusion protein is anti-inflammatory (e.g., when administered to a subject). In some embodiments, the immunomodulatory fusion protein suppresses pro-inflammatory response (e.g., when administered to a subject).

In some embodiments, the immunomodulatory fusion protein exhibits agonistic activity. In some embodiments, the immunomodulatory fusion protein exhibits antagonistic activity. In some embodiments, the immunomodulatory fusion protein exhibits both agonistic and antagonistic activity. In some embodiments, the immunomodulatory fusion protein exhibits agonistic activity on a plurality of proteins. In some embodiments, the immunomodulatory fusion protein exhibits antagonistic activity on a plurality of proteins. In some embodiments, the immunomodulatory fusion protein exhibits antagonistic activity on at least one protein and agonistic activity on at least one protein.

In some embodiments, the immunomodulatory fusion protein is capable of binding a plurality of target proteins simultaneously. In some embodiments, the immunomodulatory fusion protein is capable of inhibiting or reducing (e.g., preventing) the binding of a plurality of ligands to their respective receptor(s) (e.g., simultaneously). In some embodiments, the immunomodulatory fusion protein is capable of inhibiting or reducing (e.g., preventing) signaling mediated by the binding of a plurality of ligands to their respective receptor(s) (e.g., simultaneously).

In some embodiments, the immunomodulatory fusion protein is capable of specifically binding a plurality of integrins. In some embodiments, the immunomodulatory fusion protein is capable of specifically binding one or more integrin expressed on surface of one or more immune cell (e.g., a T cell). In some embodiments, the immunomodulatory fusion protein is capable of one or more of integrin α1β1, α2β1, α10β1, α11β1, α5β1, α8β1, α5β1, α5β3, α5β5, α5β6, α5β8, αIIbβ3, α3β1, α6β1, α7β1, 0604, α4β1, α9β1, α4β7, αEβ7, αLβ2, αMβ2, αXβ2, and/or αDβ2. In some embodiments, the immunomodulatory fusion protein is capable of specifically binding integrin α4β7.

In some embodiments, the fusion protein is capable of specifically binding hIL-10R. In some embodiments, the fusion protein is capable of specifically binding hTL1A. In some embodiments, the fusion protein is capable of specifically binding TNFα. In some embodiments, the fusion protein is capable of specifically binding CD30L.

In some embodiments, the fusion protein is capable of specifically binding hIL-10R and hTL1A. In some embodiments, the fusion protein is capable of specifically binding hIL-10R and hCD30L. In some embodiments, the fusion protein is capable of simultaneously specifically binding hTL1A and hIL-10R. In some embodiments, the fusion protein is capable of simultaneously specifically binding hTL1A and hTNFα. In some embodiments, the fusion protein is capable of simultaneously specifically binding hTL1A and hCD30L. In some embodiments, the fusion protein is capable of simultaneously specifically binding hTNFα and hIL-10R. In some embodiments, the fusion protein is capable of simultaneously specifically binding hTNFα and hTL1A. In some embodiments, the fusion protein is capable of simultaneously specifically binding hTNFα and hCD30L. In some embodiments, the fusion protein is capable of simultaneously specifically binding hCD30L and hIL-10R. In some embodiments, the fusion protein is capable of simultaneously specifically binding hCD30L and hTL1A. In some embodiments, the fusion protein is capable of simultaneously specifically binding hCD30L and hTNFα.

In some embodiments, the fusion protein is capable of simultaneously specifically binding hIL-10R, hTL1A, and TNFα. In some embodiments, the fusion protein is capable of simultaneously specifically binding hIL-10R, hTL1A, and CD30L. In some embodiments, the fusion protein is capable of simultaneously specifically binding hIL-10R, CD30L, and TNFα. In some embodiments, the fusion protein is capable of simultaneously specifically binding hTL1A, TNFα, and CD30L. In some embodiments, the fusion protein is capable of simultaneously specifically binding hIL-10R, hTL1A, TNFα, and CD30L.

In some embodiments, the immunomodulatory fusion protein is capable of binding a plurality of target proteins simultaneously. In some embodiments, the immunomodulatory fusion protein is capable of inhibiting or reducing (e.g., preventing) the binding of a plurality of ligands to their respective receptor(s) (e.g., simultaneously). In some embodiments, the immunomodulatory fusion protein is capable of inhibiting or reducing (e.g., preventing) signaling mediated by the binding of a plurality of ligands to their respective receptor(s) (e.g., simultaneously).

In some embodiments, the fusion protein is capable of inhibiting or reducing (e.g., preventing) binding of hTL1A to hDR3. In some embodiments, the fusion protein is capable of inhibiting or reducing (e.g., preventing) binding of hTNFα to hTNFR1 and/or hTNFR2. In some embodiments, the fusion protein is capable of inhibiting or reducing (e.g., preventing) binding of hCD30L to hCD30.

In some embodiments, the fusion protein is capable of simultaneously inhibiting or reducing (e.g., preventing) binding of hTL1A to hDR3 and hTNFα to hTNFR1 and/or hTNFR2.

In some embodiments, the fusion protein is capable of simultaneously inhibiting or reducing (e.g., preventing) binding of hTL1A to hDR3 and hCD30L to hCD30. In some embodiments, the fusion protein is capable of simultaneously inhibiting or reducing (e.g., preventing) binding of hTNFα to hTNFR1 and/or hTNFR2 and hTL1A to hDR3.

In some embodiments, the fusion protein is capable of simultaneously inhibiting or reducing (e.g., preventing) binding of hTNFα to hTNFR1 and/or hTNFR2 and hLTα to hTNFR1 and/or hTNFR2. In some embodiments, the fusion protein is capable of simultaneously inhibiting or reducing (e.g., preventing) binding of hTNFα to hTNFR1 and/or hTNFR2 and hCD30L to hCD30. In some embodiments, the fusion protein is capable of simultaneously inhibiting or reducing (e.g., preventing) binding of hCD30L to hCD30 and hTL1A to hDR3. In some embodiments, the fusion protein is capable of simultaneously inhibiting or reducing (e.g., preventing) binding of hCD30L to hCD30 and hTNFα to hTNFR1 and/or hTNFR2.

In some embodiments, the fusion protein is capable of simultaneously inhibiting or reducing (e.g., preventing) binding of hTL1A to hDR3, hTNFα to hTNFR1 and/or hTNFR2, and hCD30L to hCD30.

In some embodiments, the fusion protein is capable of inducing or enhancing hIL-10R (e.g., hIL-10Rβ, hIL-10Rα) signaling (e.g., signaling mediated by binding of an immunomodulatory protein described herein to hIL-10R). In some embodiments, the fusion protein is capable of inhibiting or reducing of hDR3 signaling (e.g., hDR3 signaling mediated by binding of hTL1A to hDR3). In some embodiments, the fusion protein is capable of inhibiting or reducing hTNFR1 and/or hTNFR2 signaling (e.g., hTNFR1 and/or hTNFR2 signaling mediated by binding of hTNFα to hTNFR1 and/or hTNFR2). In some embodiments, the fusion protein is capable of inhibiting or reducing hCD30 signaling (e.g., hCD30 signaling mediated by binding of hCD30L to hCD30).

In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R) and inhibiting or reducing of hDR3 signaling (e.g., hDR3 signaling mediated by binding of hTL1A to hDR3). In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R) and inhibiting or reducing hTNFR1 and/or hTNFR2 signaling (e.g., hTNFR1 and/or hTNFR2 signaling mediated by binding of hTNFα to hTNFR1 and/or hTNFR2).

In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R) and inhibiting or reducing hCD30 signaling (e.g., hCD30 signaling mediated by binding of hCD30L to hCD30). In some embodiments, the fusion protein is capable of simultaneously inhibiting or reducing of hDR3 signaling (e.g., hDR3 signaling mediated by binding of hTL1A to hDR3) and inducing or enhancing hIL-10R (e.g., hIL-10Rβ, hIL-10Rα) signaling (e.g., signaling mediated by binding of an immunomodulatory protein described herein to hIL-10R).

In some embodiments, the fusion protein is capable of simultaneously inhibiting or reducing of hDR3 signaling (e.g., hDR3 signaling mediated by binding of hTL1A to hDR3) and inhibiting or reducing hTNFR1 and/or hTNFR2 signaling (e.g., hTNFR1 and/or hTNFR2 signaling mediated by binding of hTNFα to hTNFR1 and/or hTNFR2). In some embodiments, the fusion protein is capable of simultaneously inhibiting or reducing of hDR3 signaling (e.g., hDR3 signaling mediated by binding of hTL1A to hDR3) and inhibiting or reducing hCD30 signaling (e.g., hCD30 signaling mediated by binding of hCD30L to hCD30).

In some embodiments, the fusion protein is capable of simultaneously inhibiting or reducing hTNFR1 and/or hTNFR2 signaling (e.g., hTNFR1 and/or hTNFR2 signaling mediated by binding of hTNFα to hTNFR1 and/or hTNFR2) and inducing or enhancing hIL-10R (e.g., hIL-10Rβ, hIL-10Rα) signaling (e.g., signaling mediated by binding of an immunomodulatory protein described herein to hIL-10R). In some embodiments, the fusion protein is capable of simultaneously inhibiting or reducing hTNFR1 and/or hTNFR2 signaling (e.g., hTNFR1 and/or hTNFR2 signaling mediated by binding of hTNFα to hTNFR1 and/or hTNFR2) and inhibiting or reducing of hDR3 signaling (e.g., hDR3 signaling mediated by binding of hTL1A to hDR3).

In some embodiments, the fusion protein is capable of simultaneously inhibiting or reducing hTNFR1 and/or hTNFR2 signaling (e.g., hTNFR1 and/or hTNFR2 signaling mediated by binding of hTNFα to hTNFR1 and/or hTNFR2) and/or hTNFR2 and inhibiting or reducing hCD30 signaling (e.g., hCD30 signaling mediated by binding of hCD30L to hCD30). In some embodiments, the fusion protein is capable of simultaneously inhibiting or reducing hCD30 signaling (e.g., hCD30 signaling mediated by binding of hCD30L to hCD30) and inducing or enhancing hIL-10R (e.g., hIL-10Rβ, hIL-10Rα) signaling (e.g., signaling mediated by binding of an immunomodulatory protein described herein to hIL-10R).

In some embodiments, the fusion protein is capable of simultaneously inhibiting or reducing hCD30 signaling (e.g., hCD30 signaling mediated by binding of hCD30L to hCD30) and inhibiting or reducing of hDR3 signaling (e.g., hDR3 signaling mediated by binding of hTL1A to hDR3). In some embodiments, the fusion protein is capable of simultaneously inhibiting or reducing hCD30 signaling (e.g., hCD30 signaling mediated by binding of hCD30L to hCD30) and inhibiting or reducing hTNFR1 and/or hTNFR2 signaling (e.g., hTNFR1 and/or hTNFR2 signaling mediated by binding of hTNFα to hTNFR1 and/or hTNFR2).

In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R), inhibiting or reducing of hDR3 signaling (e.g., hDR3 signaling mediated by binding of hTL1A to hDR3), and inhibiting or reducing hTNFR1 and/or hTNFR2 signaling (e.g., hTNFR1 and/or hTNFR2 signaling mediated by binding of hTNFα to hTNFR1 and/or hTNFR2). In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R), inhibiting or reducing of hDR3 signaling (e.g., hDR3 signaling mediated by binding of hTL1A to hDR3), and inhibiting or reducing hCD30 signaling (e.g., hCD30 signaling mediated by binding of hCD30L to hCD30).

In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R), inhibiting or reducing hCD30 signaling (e.g., hCD30 signaling mediated by binding of hCD30L to hCD30), and inhibiting or reducing hTNFR1 and/or hTNFR2 signaling (e.g., hTNFR1 and/or hTNFR2 signaling mediated by binding of hTNFα to hTNFR1 and/or hTNFR2). In some embodiments, the fusion protein is capable of simultaneously inhibiting or reducing of hDR3 signaling (e.g., hDR3 signaling mediated by binding of hTL1A to hDR3 inhibiting or reducing hTNFR1 and/or hTNFR2 signaling (e.g., hTNFR1 and/or hTNFR2 signaling mediated by binding of hTNFα to hTNFR1 and/or hTNFR2), and inhibiting or reducing hCD30 signaling (e.g., hCD30 signaling mediated by binding of hCD30L to hCD30).

In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R), inhibiting or reducing of hDR3 signaling (e.g., hDR3 signaling mediated by binding of hTL1A to hDR3), inhibiting or reducing hTNFR1 and/or hTNFR2 signaling (e.g., hTNFR1 and/or hTNFR2 signaling mediated by binding of hTNFα to hTNFR1 and/or hTNFR2), and inhibiting or reducing hCD30 signaling (e.g., hCD30 signaling mediated by binding of hCD30L to hCD30).

In some embodiments, the fusion protein is capable of simultaneously specifically binding one or more integrin (e.g., integrin α4β7), hIL-10R, and hTL1A. In some embodiments, the fusion protein is capable of simultaneously specifically binding one or more integrin (e.g., integrin α4β7), hTL1A, and hIL-10R. In some embodiments, the fusion protein is capable of simultaneously specifically binding one or more integrin (e.g., integrin α4β7), hTL1A, and hTNFα. In some embodiments, the fusion protein is capable of simultaneously specifically binding one or more integrin (e.g., integrin α4β7), hTNFα, and hIL-10R. In some embodiments, the fusion protein is capable of simultaneously specifically binding one or more integrin (e.g., integrin α4β7), hTNFα, and hTL1A. In some embodiments, the fusion protein is capable of simultaneously specifically binding one or more integrin (e.g., integrin α4β7), hIL-10R, hTL1A, and TNFα.

In some embodiments, the fusion protein is capable of simultaneously specifically binding integrin α4β7, hIL-10R, and hTL1A. In some embodiments, the fusion protein is capable of simultaneously specifically binding integrin α4β7, hTL1A, and hIL-10R. In some embodiments, the fusion protein is capable of simultaneously specifically binding integrin α4β7, hTL1A, and hTNFα. In some embodiments, the fusion protein is capable of simultaneously specifically binding integrin α4β7, hTNFα, and hIL-10R. In some embodiments, the fusion protein is capable of simultaneously specifically binding integrin α4β7, hTNFα, and hTL1A. In some embodiments, the fusion protein is capable of simultaneously specifically binding integrin α4β7, hIL-10R, hTL1A, and TNFα.

In some embodiments, the fusion protein is capable of simultaneously inhibiting or reducing (e.g., preventing) binding of one or more integrin to its cognate partner. In some embodiments, the fusion protein is capable of simultaneously inhibiting or reducing (e.g., preventing) binding of integrin α4β7 to MADCAM-1.

In some embodiments, the fusion protein is capable of simultaneously inhibiting or reducing (e.g., preventing) binding of one or more integrin to its cognate partner and hIL-10 to hIL-10R (e.g., hIL-10Rβ, hIL-10Rα). In some embodiments, the fusion protein is capable of simultaneously inhibiting or reducing (e.g., preventing) binding of one or more integrin to its cognate partner and hTL1A to hDR3. In some embodiments, the fusion protein is capable of simultaneously inhibiting or reducing (e.g., preventing) binding of one or more integrin to its cognate partner and hTNFα to hTNFR1 and/or hTNFR2.

In some embodiments, the fusion protein is capable of simultaneously inhibiting or reducing (e.g., preventing) binding of one or more integrin to its cognate partner, hIL-10 to hIL-10R (e.g., hIL-10Rβ, hIL-10Rα), and hTL1A to hDR3. In some embodiments, the fusion protein is capable of simultaneously inhibiting or reducing (e.g., preventing) binding of one or more integrin to its cognate partner, hIL-10 to hIL-10R (e.g., hIL-10Rβ, hIL-10Rα), and hTNFα to hTNFR1 and/or hTNFR2. In some embodiments, the fusion protein is capable of simultaneously inhibiting or reducing (e.g., preventing) binding of one or more integrin to its cognate partner, hTL1A to hDR3, and hIL-10 to hIL-10R (e.g., hIL-10Rβ, hIL-10Rα). In some embodiments, the fusion protein is capable of simultaneously inhibiting or reducing (e.g., preventing) binding of one or more integrin to its cognate partner, hTL1A to hDR3, and hTNFα to hTNFR1 and/or hTNFR2. In some embodiments, the fusion protein is capable of simultaneously inhibiting or reducing (e.g., preventing) binding of one or more integrin to its cognate partner, hTNFα to hTNFR1, and/or hTNFR2 and hIL-10 to hIL-OR (e.g., hIL-10Rβ, hIL-10Rα). In some embodiments, the fusion protein is capable of simultaneously inhibiting or reducing (e.g., preventing) binding of one or more integrin to its cognate partner, hTNFα to hTNFR1, and/or hTNFR2 and hTL1A to hDR3. In some embodiments, the fusion protein is capable of simultaneously inhibiting or reducing (e.g., preventing) binding of one or more integrin to its cognate partner, hTNFα to hTNFR1, and/or hTNFR2 and hLTα to hTNFR1 and/or hTNFR2. In some embodiments, the fusion protein is capable of simultaneously inhibiting or reducing (e.g., preventing) binding of one or more integrin to its cognate partner, hIL-10 to hIL-10R (e.g., hIL-10Rβ, hIL-10Rα), hTL1A to hDR3, and hTNFα to hTNFR1 and/or hTNFR2.

In some embodiments, the fusion protein is capable of simultaneously inhibiting or reducing (e.g., preventing) binding of integrin α4β7 to MADCAM-1 and hIL-10 to hIL-10R (e.g., hIL-10Rβ, hIL-10Rα). In some embodiments, the fusion protein is capable of simultaneously inhibiting or reducing (e.g., preventing) binding of integrin α4β7 to MADCAM-1 and hTL1A to hDR3. In some embodiments, the fusion protein is capable of simultaneously inhibiting or reducing (e.g., preventing) binding of integrin α4β7 to MADCAM-1 and hTNFα to hTNFR1 and/or hTNFR2.

In some embodiments, the fusion protein is capable of simultaneously inhibiting or reducing (e.g., preventing) binding of integrin α4β7 to MADCAM-1, hIL-10 to hIL-10R (e.g., hIL-10Rβ, hIL-10Rα), and hTL1A to hDR3. In some embodiments, the fusion protein is capable of simultaneously inhibiting or reducing (e.g., preventing) binding of integrin α4β7 to MADCAM-1, hIL-10 to hIL-10R (e.g., hIL-10Rβ, hIL-10Rα), and hTNFα to hTNFR1 and/or hTNFR2. In some embodiments, the fusion protein is capable of simultaneously inhibiting or reducing (e.g., preventing) binding of integrin α4β7 to MADCAM-1, hTL1A to hDR3, and hIL-10 to hIL-10R (e.g., hIL-10Rβ, hIL-10Rα). In some embodiments, the fusion protein is capable of simultaneously inhibiting or reducing (e.g., preventing) binding of integrin α4β7 to MADCAM-1, hTL1A to hDR3, and hTNFα to hTNFR1 and/or hTNFR2. In some embodiments, the fusion protein is capable of simultaneously inhibiting or reducing (e.g., preventing) binding of integrin α4β7 to MADCAM-1, hTNFα to hTNFR1, and/or hTNFR2 and hIL-10 to hIL-10R (e.g., hIL-10Rβ, hIL-10Rα). In some embodiments, the fusion protein is capable of simultaneously inhibiting or reducing (e.g., preventing) binding of integrin α4β7 to MADCAM-1, hTNFα to hTNFR1, and/or hTNFR2 and hTL1A to hDR3. In some embodiments, the fusion protein is capable of simultaneously inhibiting or reducing (e.g., preventing) binding of integrin α4β7 to MADCAM-1, hTNFα to hTNFR1, and/or hTNFR2 and hLTα to hTNFR1 and/or hTNFR2. In some embodiments, the fusion protein is capable of simultaneously inhibiting or reducing (e.g., preventing) binding of integrin α4β7 to MADCAM-1, hIL-10 to hIL-10R (e.g., hIL-10Rβ, hIL-10Rα), hTL1A to hDR3, and hTNFα to hTNFR1 and/or hTNFR2.

In some embodiments, the fusion protein is capable of inhibiting or reducing signaling by a cognate partner of an integrin (e.g., signaling mediated by binding of the integrin to the cognate partner). In some embodiments, the fusion protein is capable of inhibiting or reducing (e.g., preventing) MADCAM-1 signaling (e.g., signaling mediated by the binding of MADCM-1 to integrin α4β7). In some embodiments, the fusion protein is capable of inhibiting or reducing signaling mediated by the binding of one or more integrin to its cognate partner. In some embodiments, the fusion protein is capable of inhibiting or reducing signaling mediated by the binding of integrin α4β7 to MADCAM-1.

In some embodiments, the fusion protein is capable of inducing or enhancing hIL-10R (e.g., hIL-10Rβ, hIL-10Rα) signaling (e.g., signaling mediated by binding of an immunomodulatory protein described herein to hIL-10R) and inhibiting or reducing signaling by a cognate partner of an integrin (e.g., signaling mediated by binding of the integrin to the cognate partner (e.g., signaling mediated by binding of integrin α4β7 to MADCAM-1)). In some embodiments, the fusion protein is capable of inhibiting or reducing of hDR3 signaling (e.g., hDR3 signaling mediated by binding of hTL1A to hDR3) and inhibiting or reducing signaling by a cognate partner of an integrin (e.g., signaling mediated by binding of the integrin to the cognate partner (e.g., signaling mediated by binding of integrin α4β7 to MADCAM-1)). In some embodiments, the fusion protein is capable of inhibiting or reducing hTNFR1 and/or hTNFR2 signaling (e.g., hTNFR1 and/or hTNFR2 signaling mediated by binding of hTNFα to hTNFR1 and/or hTNFR2) and inhibiting or reducing signaling by a cognate partner of an integrin (e.g., signaling mediated by binding of the integrin to the cognate partner (e.g., signaling mediated by binding of integrin α4β7 to MADCAM-1)).

In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R), inhibiting or reducing of hDR3 signaling (e.g., hDR3 signaling mediated by binding of hTL1A to hDR3), and inhibiting or reducing signaling by a cognate partner of an integrin (e.g., signaling mediated by binding of the integrin to the cognate partner (e.g., signaling mediated by binding of integrin α4β7 to MADCAM-1)). In some embodiments, the fusion protein is capable of simultaneously inducing or enhancing hIL-10R (e.g., hIL-10R3, hIL-10Rα) signaling (e.g., signaling mediated by binding of an immunomodulatory protein described herein to hIL-10R), inhibiting or reducing hTNFR1 and/or hTNFR2 signaling (e.g., hTNFR1 and/or hTNFR2 signaling mediated by binding of hTNFα to hTNFR1 and/or hTNFR2), and inhibiting or reducing signaling by a cognate partner of an integrin (e.g., signaling mediated by binding of the integrin to the cognate partner (e.g., signaling mediated by binding of integrin α4β7 to MADCAM-1)).

In some embodiments, the fusion protein is capable of simultaneously inhibiting or reducing of hDR3 signaling (e.g., hDR3 signaling mediated by binding of hTL1A to hDR3), inducing or enhancing hIL-10R (e.g., hIL-10R3, hIL-10Rα) signaling (e.g., signaling mediated by binding of an immunomodulatory protein described herein to hIL-10R), and inhibiting or reducing signaling by a cognate partner of an integrin (e.g., signaling mediated by binding of the integrin to the cognate partner (e.g., signaling mediated by binding of integrin α4β7 to MADCAM-1)). In some embodiments, the fusion protein is capable of simultaneously inhibiting or reducing of hDR3 signaling (e.g., hDR3 signaling mediated by binding of hTL1A to hDR3), inhibiting or reducing hTNFR1 and/or hTNFR2 signaling (e.g., hTNFR1 and/or hTNFR2 signaling mediated by binding of hTNFα to hTNFR1 and/or hTNFR2), and inhibiting or reducing signaling by a cognate partner of an integrin (e.g., signaling mediated by binding of the integrin to the cognate partner (e.g., signaling mediated by binding of integrin α4β7 to MADCAM-1)).

In some embodiments, the fusion protein is capable of simultaneously inhibiting or reducing hTNFR1 and/or hTNFR2 signaling (e.g., hTNFR1 and/or hTNFR2 signaling mediated by binding of hTNFα to hTNFR1 and/or hTNFR2), inducing or enhancing hIL-10R (e.g., hIL-10Rβ, hIL-10Rα) signaling (e.g., signaling mediated by binding of an immunomodulatory protein described herein to hIL-10R), and inhibiting or reducing signaling by a cognate partner of an integrin (e.g., signaling mediated by binding of the integrin to the cognate partner (e.g., signaling mediated by binding of integrin α4β7 to MADCAM-1)). In some embodiments, the fusion protein is capable of simultaneously inhibiting or reducing hTNFR1 and/or hTNFR2 signaling (e.g., hTNFR1 and/or hTNFR2 signaling mediated by binding of hTNFα to hTNFR1 and/or hTNFR2), inhibiting or reducing of hDR3 signaling (e.g., hDR3 signaling mediated by binding of hTL1A to hDR3), and inhibiting or reducing signaling by a cognate partner of an integrin (e.g., signaling mediated by binding of the integrin to the cognate partner (e.g., signaling mediated by binding of integrin α4β7 to MADCAM-1)).

In some embodiments, the fusion protein is capable of simultaneously inducing or enhancing hIL-10R (e.g., hIL-10Rβ, hIL-10Rα) signaling (e.g., signaling mediated by binding of an immunomodulatory protein described herein to hIL-10R), inhibiting or reducing of hDR3 signaling (e.g., hDR3 signaling mediated by binding of hTL1A to hDR3), inhibiting or reducing hTNFR1 and/or hTNFR2 signaling (e.g., hTNFR1 and/or hTNFR2 signaling mediated by binding of hTNFα to hTNFR1 and/or hTNFR2), and inhibiting or reducing signaling by a cognate partner of an integrin (e.g., signaling mediated by binding of the integrin to the cognate partner (e.g., signaling mediated by binding of integrin α4β7 to MADCAM-1)).

Binding and binding affinity can be measured by standard assays known in the art. For example, binding affinity can be measured by surface plasmon resonance (SPR) (e.g., BIAcore®-based assay), a common method known in the art (see, e.g., Wilson, Science 295:2103, 2002; Wolff et al., Cancer Res. 55:2560, 1993; and U.S. Pat. Nos. 5,283,173, 5,468,614, the full contents of each of which are incorporated by reference herein for all purposes). SPR measures changes in the concentration of molecules at a sensor surface as molecules bind to or dissociate from the surface. The change in the SPR signal is directly proportional to the change in mass concentration close to the surface, thereby allowing measurement of binding kinetics between two molecules (e.g., proteins). The dissociation constant for the complex can be determined by monitoring changes in the refractive index with respect to time as buffer is passed over the chip.

Other suitable assays for measuring the binding of one protein to another (e.g., binding of a protein described herein to a TNFSF ligand) include, for example, immunoassays such as enzyme linked immunosorbent assays (ELISA) and radioimmunoassays (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, analytical ultracentrifugation, spectroscopy, flow cytometry, sequencing and other methods for detection of binding of proteins.

### 5.10 Conjugates & Additional Fusion Proteins

In some embodiments, a fusion protein described herein is operably connected to a heterologous moiety forming a conjugate or a fusion protein, respectively. As such, provided herein are, inter alia, conjugates comprising a fusion protein described herein (or a nucleic acid molecule encoding the same) and further comprising one or more heterologous moieties. Further provided herein are, inter alia, additional fusion proteins comprising a fusion protein described herein (e.g., described above) and further comprising one or more heterologous proteins (or a functional fragment, functional variant, or domain thereof).

Heterologous moieties include, but are not limited to, proteins, peptides, small molecules, nucleic acid molecules (e.g., DNA, RNA, DNA/RNA hybrid molecules), carbohydrates, lipids, and synthetic polymers (e.g., polymers of PEG). In some embodiments, the heterologous moiety is a detectable moiety (e.g., protein, e.g., a fluorescent protein). In some embodiments, the heterologous moiety is a therapeutic agent. In some embodiments, the heterologous moiety is an imaging agent. In some embodiments, the heterologous moiety is a radioligand. In some embodiments, the heterologous moiety is a diagnostic agent. In some embodiments, the heterologous moiety is non-effector moiety, e.g., a protein sequence that acts as a “handle” or linker but has otherwise no independent biological effect. In some embodiments, the heterologous moiety comprises an antibody, an antibody mimetic, or one or more Ig constant region (Fc region). The heterologous moiety can be any one or more of (any combination of) the foregoing.

### 5.10.1 Radioligands

In some embodiments, the heterologous moiety comprises a radioisotope. As such, provided herein are radioligands comprising an immunomodulatory fusion protein (e.g., described herein) operably connected (e.g., through a linker) to one more radioisotope. In some embodiments, the radioisotope acts as a therapeutic agent. In some embodiments, the radioisotope acts as an imaging agent. In some embodiments, the immunomodulatory fusion protein (e.g., described herein) acts as a targeting moiety for the radioisotope. In some embodiments, the radioisotope and the immunomodulatory fusion protein (e.g., described herein) are operably connected through a linker.

Radioisotopes are known in the art. See, e.g., Sgouros, G., Bodei, L., McDevitt, M. R. et al. Radiopharmaceutical therapy in cancer: clinical advances and challenges. Nat Rev Drug Discov 19, 589-608 (2020). https://doi.org/10.1038/s41573-020-0073-9; and Zhang, Longjiang et al. “Delivery of therapeutic radioisotopes using nanoparticle platforms: potential benefit in systemic radiation therapy.” Nanotechnology, science and applications vol. 3 159-70. 3 Dec. 2010, doi:10.2147/NSA.S7462; the entire contents of each of which are incorporated herein by reference for all purposes.

Exemplary radioisotopes include, but are not limited to, Lutetium-177, Radium-223, Iodine-131, Iodine-125, Fluorine-18, Ir-192, Xenon-133, Yttrium-90, Carbon-11, Idium-111, Strontium-89, Copper-67, Copper-64, Rhenium-186, Actinium-225, Astatine-211, Bismuth-213, Bismuth-212, Samarium-153, Holmium-166, Thorium-227, and Lead-212.

Methods of operably connecting proteins to radionuclides (e.g., through one or more linkers) are known in the art. See, e.g., Gupta, Suprit et al. “Antibody labeling with radioiodine and radiometals.” Methods in molecular biology (Clifton, N.J.) vol. 1141 (2014): 147-57. doi:10.5007/978-1-4939-0363-4_9; Marion Chomet, State of the Art in Radiolabeling of Antibodies with Common and Uncommon Radiometals for Preclinical and Clinical Immuno-PET, Bioconjugate Chem. 2021, 32, 7, 1315-1330; Martina Steiner, Dario Neri; Antibody-Radionuclide Conjugates for Cancer Therapy: Historical Considerations and New Trends. Clin Cancer Res 15 Oct. 2011; 17 (20): 6406-6416. https://doi.org/10.1158/1078-0432.CCR-11-0483; the entire contents of each of which are incorporated herein by reference for all purposes.

### 5.10.2 Chimeric Antigen Receptors

In some embodiments, a fusion protein described herein is part of a chimeric antigen receptor (CAR). In some embodiments, a fusion protein described herein is the extracellular antigen-binding domain of a CAR. Standard CAR domains are known in art, including, e.g., transmembrane domains and intracellular signaling domains. See, e.g., WO2024056809, WO2023240064A1, and WO2023205148A1, WO2023133092A1, the entire contents of each of which is incorporated herein by reference for all purposes.

Exemplary transmembrane domains include, e.g., the alpha, beta or zeta chain of T-cell receptor, CD28, CD3 epsilon, CD45, CD4, CD5, CD8 (for example, CD8 alpha, CD8 beta), CD9, CD 16, CD22, CD33, CD37, CD64, CD80, CD86, CD134, CD137, CD154. In some embodiments, a transmembrane domain may include at least the transmembrane region(s) of a costimulatory molecule, for example, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD500 (SEMA4D), CD69, SLAMF6 (NTB-A, Ly108), SLAM (SLAMF1, CD 150, IPO-3), BLAME (SLAMF8), SELPLG (CD 162), LTBR, LAT, GADS, SLP-76, PAG/Cbp, CD 19a, and a ligand that specifically binds with CD83. In some instances, the transmembrane domain can be attached to the extracellular region of the CAR, for example, the antigen-binding domain of the CAR, via a hinge, for example, a hinge from a human protein. For example, in some embodiments, the hinge can be a human Ig (immunoglobulin) hinge, for example, an IgG4 hinge, or a CD8a hinge.

Exemplary intracellular signaling domains include, e.g., the cytoplasmic sequences of the T cell receptor (TCR) and co-receptors that act in concert to initiate signal transduction following antigen receptor engagement, as well as any derivative or variant of these sequences and any recombinant sequence that has the same functional capability. In some embodiments, the intracellular signaling domain comprises a primary signaling domain and one or more costimulatory signaling domain. Exemplary primary signaling domains, include, e.g., intracellular signaling domains of TCR zeta, FcR gamma, FcR beta, CD3 gamma, CD3 delta, CD3 epsilon, CD5, CD22, CD79a, CD79b, CD278 (also known as “ICOS”), FccRI, DAP10, DAP12, CD32, and CD66d. Exemplary of proteins with costimulatory domains suitable for use in CAR described herein include, e.g., MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, 0X40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD 11d, ITGAE, CD 103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRT AM, Ly9 (CD229), CD 160 (BY55), PSGL1, CD500 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, CD 19a, and a ligand that specifically binds with CD83, and the like.

### 5.11 Methods of Making Proteins

Any protein described herein, e.g., any fusion protein (or protein component thereof) may be produced using standard methods known in the art. For example, any fusion protein (or protein component thereof) may be produced by recombinant technology in host cells (e.g., yeast cells, insect cells, mammalian cells, bacteria) that have been transfected or transduced with one or more nucleic acid expression vector (e.g., plasmid, viral vector (e.g., a baculoviral expression vector)) encoding the subject protein (e.g., fusion protein (or any component thereof)). Such general methods are common knowledge in the art. The expression vector typically contains an expression cassette that includes nucleic acid sequences capable of bringing about expression of the nucleic acid molecule encoding the protein of interest, such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the protein of interest are cultured under conditions conducive to expression of the nucleic acid molecule encoding the protein of interest. Culture media is available from various vendors, and a suitable medium can be routinely chosen for a host cell to express a protein of interest. Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The protein may be concentrated, buffer exchanged by ultrafiltration, and the retentate from the ultrafiltration may be filtered through an appropriate filter, e.g., a 0.22 μm filter. See, e.g., Hacker, David (Ed.), Recombinant Protein Expression in Mammalian Cells: Methods and Protocols (Methods in Molecular Biology), Humana Press (2018); and McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes.

The proteins described herein (e.g., fusion proteins (or any component thereof)) may be produced synthetically. The proteins described herein (e.g., including the fusion proteins) may be produced by using an egg-based manufacturing method. In some embodiments, the proteins described herein (e.g., fusion proteins described herein) are produced in yeast.

In some aspects and embodiments, the disclosure features methods of making a protein described herein (e.g., a fusion protein (or any component thereof)). The method includes (a) recombinantly expressing the protein (e.g., a fusion protein described herein); (b) enriching, e.g., purifying, the protein (e.g., the fusion); (c) evaluating the protein (e.g., the fusion protein) for the presence of a process impurity or contaminant, and (d) formulating the protein (e.g., the fusion protein described herein) as a pharmaceutical composition if the protein (e.g., the fusion protein described herein) meets a threshold specification for the process impurity or contaminant. The process impurity or contaminant evaluated may be one or more of, e.g., a process-related impurity such as host cell proteins, host cell DNA, or a cell culture component (e.g., inducers, antibiotics, or media components); a product-related impurity (e.g., precursors, fragments, aggregates, degradation products); or contaminants, e.g., endotoxin, bacteria, viral contaminants.

### 5.12 Nucleic Acid Molecules

In one aspect, provided herein are nucleic acid molecules (e.g., DNA molecules, RNA molecules, hybrid RNA/DNA molecules) encoding any protein described herein (including, e.g., a fusion protein described herein (or any component thereof)). In some embodiments, the nucleic acid molecule is a DNA molecule or an RNA molecule.

In some embodiments, the nucleic acid molecule is codon optimized. Codon optimization may be used to match codon frequencies in target and host organisms to ensure proper folding; bias guanosine (G) and/or cytosine content to increase nucleic acid stability; minimize tandem repeat codons or base runs that may impair gene construction or expression; customize transcriptional and translational control regions; insert or remove protein trafficking sequences; remove/add post translation alteration sites in encoded protein (e.g., glycosylation sites); add, remove, or shuffle protein domains; insert or delete restriction sites; modify ribosome binding sites and mRNA degradation sites; adjust translational rates to allow the various domains of the protein to fold properly; or to reduce or eliminate problem secondary structures within the nucleic acid molecule. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms. In some embodiments, the nucleic acid sequence is modified to optimize the number of G and/or C nucleotides as compared to a reference nucleic acid sequence. An increase in the number of G and C nucleotides may be generated by substitution of codons containing adenosine (T) or thymidine (T) (or uracil (U)) nucleotides by codons containing G or C nucleotides.

### 5.12.1 DNA Molecules

In some embodiments, the nucleic acid molecule is a DNA molecule. In some embodiments, the DNA molecule is a linear coding DNA construct, contained within a plasmid, or contained within a viral vector. In some embodiments, the DNA molecule is a linear coding DNA construct. In some embodiments, the DNA molecule is contained within a plasmid. In some embodiments, the DNA molecule is contained with a viral vector. A more detailed description of viral vectors for both RNA and DNA molecules is provided in § 5.13.2.

The coding DNA may also comprise one or more heterologous nucleic acid elements to mediate expression of the coding region. These include, e.g., promoter(s), enhancer(s), polyadenylation signal(s), synthetic introns, transcriptional termination signals, polyadenylation sequences, and other transcription regulatory elements. A person of ordinary skill in the art is familiar with the transcriptional regulatory elements needed for expression of the coding DNA and can optimize the expression construct (e.g., linear DNA or a plasmid) accordingly.

In some embodiments, a promoter is operably linked to the respective coding nucleic acid sequence encoding a fusion protein (or polypeptide thereof) described herein. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the entire contents of which is incorporated by reference herein for all purposes. Exemplary polyadenylation signals, include, but are not limited to, the bovine growth hormone (BGH) polyadenylation site, SV40 polyadenylation signals, and LTR polyadenylation signals.

In some embodiments, the DNA is contained within a plasmid. A person of ordinary skill in the art is aware of suitable plasmids for expression of the DNA of interest. For example, Suitable plasmid DNA may be generated to allow efficient production of the encoded fusion proteins in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

### 5.12.2 RNA Molecules

In some embodiments, the nucleic acid molecule is an RNA molecule. In some embodiments, the RNA molecule is a translatable RNA molecule. In some embodiments, the RNA is selected from an mRNA, a self-replicating RNA, a circular RNA (e.g., a covalently closed RNA), a viral RNA, or a replicon RNA.

In some embodiments, the RNA molecule a circular RNA. Exemplary circular RNAs are described in e.g., U.S. Ser. No. 11/458,156, US20220143062, US20230212629, US20230072532, U.S. Ser. No. 11/203,767, U.S. Ser. No. 11/352,641, US20210371494, U.S. Ser. No. 11/766,449, US20230226096, WO2021189059, US20190345503, US20220288176, U.S. Ser. No. 11/560,567, WO2022271965, WO2022037692, WO2023024500, WO2023115732, WO2023133684, WO2023143541, WO2023134611, and WO2022247943, the entire contents of each of which are incorporated herein by reference for all purposes.

In some embodiments, the RNA is a mRNA. The basic components of an mRNA molecule typically include at least one coding region (herein a coding region encoding at least one fusion protein (or polypeptide thereof) described herein), a 5′-untranslated region (UTR), a 3′-UTR, a 5′ cap, and a poly-A tail.

In some embodiments, the RNA (e.g., mRNA, circular RNA) comprises at least one heterologous UTR. The UTRs may harbor regulatory sequence elements that determine the RNA (e.g., mRNA) turnover, stability, localization, and/or expression of operably linked coding sequence(s). The heterologous UTRs may be derived from a naturally occurring genes or may be synthetically engineered. In some embodiments, the 5′-UTR comprises elements for controlling gene expression, e.g., ribosomal binding sites, miRNA binding sites. The 5′-UTR may be post-transcriptionally modified or varied, e.g., by enzymatic or post-transcriptional addition of a 5′cap structure. In some embodiments, the 3′-UTR comprises a polyadenylation signal. In some embodiments, the RNA (e.g., mRNA, circular RNA) comprises at least one coding region encoding a fusion protein (or polypeptide thereof) described herein and 5′-UTR and/or a 3′-UTR. In some embodiments, the RNA (e.g., mRNA, circular RNA) comprises at least one coding sequence encoding a fusion protein (or polypeptide thereof) described herein operably connected to at least one heterologous 5′-UTR and at least one 3′-UTR.

In some embodiments, the RNA (e.g., mRNA, circular RNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides. In some embodiments, poly(A) sequence comprises at least 10, 20, 30, 40, 50, 60, 70, 80, 90, 500, 200, 300, 400, or 500 adenosine nucleotides. In some embodiments, the RNA (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides, wherein the 3′ terminal nucleotide of said nucleic acid molecule is an adenosine. In some embodiments, poly(A) sequence comprises at least 10, 20, 30, 40, 50, 60, 70, 80, 90, 500, 200, 300, 400, or 500 adenosine nucleotides, wherein the 3′ terminal nucleotide of said nucleic acid molecule is an adenosine.

In some embodiments, the RNA (e.g., mRNA, circular RNA) comprises a poly(A)(U) sequence. In some embodiments, the RNA (e.g., mRNA, circular RNA) comprises a poly(A)(C) sequence.

In some embodiments, the RNA (e.g., mRNA) comprises a 5′-cap structure. In some embodiments, the 5′-cap structure stabilizes the RNA (e.g., mRNA), enhances expression of the encoded fusion protein (or polypeptide thereof) described herein, and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphorothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine. In some embodiments, the 5′ cap structure comprises m7G, cap0, cap1, cap2, a modified capO, or a modified cap1 structure.

In some embodiments, the RNA (e.g., mRNA, circular RNA) comprises nucleotide analogues/modifications, e.g., backbone modifications, sugar modifications, and/or base modifications. A backbone modification in the context of the present disclosure is a modification, in which phosphates of the backbone of the nucleotides of the RNA (e.g., mRNA) are chemically modified. A sugar modification in the context of the present disclosure is a chemical modification of the sugar of the nucleotides of the RNA (e.g., mRNA). A base modification in the context of the present disclosure is a chemical modification of the base moiety of the nucleotides of the RNA (e.g., mRNA).

In some embodiments, the RNA (e.g., mRNA, circular RNA) comprises at least one modified nucleotide. Exemplary nucleotide analogues/modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

RNA (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in the art. For example, using PureMessenger® (CureVac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.13 Vectors

In one aspect, provided herein are vectors comprising a nucleic acid molecule (e.g., DNA molecule, RNA molecule) described herein (e.g., a nucleic acid molecule described in § 5.12) (e.g., a nucleic acid molecule encoding a fusion protein described herein (or one or more component thereof). In some embodiments, the vector is linear. In some embodiments, the vector is a viral vector. In some embodiments, the vector is a non-viral vector (e.g., a plasmid, a minicircle).

### 5.13.1 Non-Viral Vectors

In some embodiments, the vector is a non-viral vector. In some embodiments, the vector is a minicircle. In some embodiments, the vector is a plasmid. A person of ordinary skill in the art is aware of suitable plasmids for expression of the DNA of interest. For example, plasmid DNA may be generated to allow efficient production of the encoded endonucleases in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

### 5.13.2 Viral Vectors

In some embodiments, the nucleic acid molecules (e.g., DNA or RNA) are contained in a viral vector. Thus, also provided herein are viral vectors comprising the nucleic acid molecules described herein. Such vectors can be easily manipulated by methods well known to the ordinary person of skill in the art. The vector used can be any vector that is suitable for cloning nucleic acids that can be used for transcription of the nucleic acid molecule of interest.

Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, lentivirus vector, anellovector (as described, for example, in U.S. Pat. No. 11,446,344, the entire contents of which is incorporated by reference herein for all purposes).

In some embodiments, the vector is an adenoviral vector (e.g., human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated by reference herein for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated by reference herein for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the transgene (e.g., a fusion protein (or polypeptide thereof) described herein) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™, pCR3.1™, pCMV™, pFRT™, pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, or by electroporation,). The cells are cultured under conditions suitable and for a sufficient time for plasmid expression. The viral particles may be purified from the cell culture medium using standard methods known to the person of ordinary skill in the art. For example, by centrifugation followed by e.g., chromatography or ultrafiltration.

In some embodiments, a fusion protein described herein, can be incorporated into a viral particle for e.g., targeting of a viral particle encoding a gene therapy cassette to a specific location within a subject (e.g., a specific cell, tissue, or organ). As such, further provided herein are, inter alia, viral particles displaying on a fusion protein described herein. Suitable methods of incorporating targeting proteins into viral particles are known in the art, including e.g., genetic fusion to viral capsid or envelope proteins, complexing with bispecific adapters, and chemical linkage. See, e.g., Nettelbeck, Dirk M. “Bispecific Antibodies and Gene Therapy.” Bispecific Antibodies 327-347. 1 Jul. 2011, doi:10.5007/978-3-642-20910-9_18, the entire contents of which are incorporated herein by reference for all purposes.

### 5.14 Cells

In one aspect, provided herein are cells (e.g., host cells) comprising any one or more of a fusion protein described herein, a conjugate described herein, a nucleic acid molecule described herein (e.g., a nucleic acid molecule encoding a fusion protein described herein), a vector described herein (e.g., comprising a nucleic acid molecule encoding a fusion protein described herein), or a carrier described herein (a carrier comprising a fusion protein described herein or a nucleic acid molecule encoding a fusion protein described herein).

In some embodiments, the cell is a eukaryotic cell. In some embodiments, the cell is mammalian cell. In some embodiments, the cell is an animal cell. In some embodiments, the cell is a human cell. In some embodiments, the cell is in vitro. In some embodiments, the cell is in vivo. In some embodiments, the cell is ex vivo.

Standard methods known in the art can be utilized to deliver any one of the foregoing (e.g., fusion protein, vector, nucleic acid molecule, carrier, etc.) into a cell (e.g., a host cell). Standard methods known in the art can be utilized to culture cells (e.g., host cells) in vitro or ex vivo.

In some embodiments, the cell (or population of cells) expresses a protein comprising a fusion protein described herein. In some embodiments, the cell (or population of cells) has been genetically engineered to comprise (e.g., within the cell's genome) a nucleic acid molecule (e.g., described herein) that encodes a fusion protein described herein. In some embodiments, the cell (or population of cells) expresses a protein comprising a fusion protein described herein on the surface of the cell.

In some embodiments, the cell (or population of cells) is a therapeutic cell. In some embodiments, the therapeutic cell (or population of cells) has been genetically engineered to comprise (e.g., within the cell's genome) a nucleic acid molecule (e.g., described herein) that encodes a fusion protein described herein. In some embodiments, the therapeutic cell (or population of cells) expresses a protein comprising a fusion protein described herein on the surface of the cell. In some embodiments, a fusion protein described herein on the surface of the cell acts as a targeting moiety. In some embodiments, the therapeutic cell is an immune cell. In some embodiments, the therapeutic cell is a T cell (e.g., a CD8+ T cell, a CD4+ T cell). In some embodiments, the therapeutic cell is a natural killer cell.

In some embodiments, the cell expresses and/or encodes a chimeric antigen receptor comprising a fusion protein (e.g., described herein) (also referred to herein as a CAR cell). As such, in one aspect, provided herein are cells expressing and/or genetically encoding a chimeric antigen receptor comprising a fusion protein described herein (e.g., utilized as the antigen binding domain of the extracellular domain of the chimeric antigen receptor). In some embodiments, the cell is a T cell (e.g., a CD8+ T cell, a CD4+ T cell). In some embodiments, the cell is a natural killer cell.

### 5.15 Carriers

Any agent described herein, including, e.g., a fusion protein described herein, a nucleic acid molecule described herein (e.g., a nucleic acid molecule encoding a fusion protein described herein), a vector described herein (e.g., a vector comprising a nucleic acid molecule (e.g., described herein) encoding a fusion protein described herein), or a conjugate described herein, can be formulated within a carrier, and/or conjugated to a carrier (e.g., as a targeting moiety).

Exemplary carriers include, but are not limited to, lipid-based carriers (e.g., lipid nanoparticles (LNPs), liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid. Lipid based carriers are further described below in § 5.15.3.

### 5.15.1 Carriers of Immunomodulatory Fusion Proteins

In some embodiments, a fusion protein described herein, a nucleic acid molecule described herein (e.g., encoding a fusion protein described herein), or a vector described herein (e.g., comprising a nucleic acid molecule (e.g., described herein) encoding a fusion protein described herein), or a conjugate described herein, is formulated within a carrier.

As, such, the disclosure provides, inter alia, carriers comprising any one of more of a fusion protein described herein, a nucleic acid molecule described herein (e.g., encoding a fusion protein described herein), or a vector described herein (e.g., comprising a nucleic acid molecule (e.g., described herein) encoding a fusion protein described herein), or a conjugate described herein.

Any of the foregoing agents described herein (e.g., proteins, nucleic acid molecules, vectors, etc.) described herein can be encapsulated within a carrier, chemically conjugated to a carrier, associated with the carrier. In this context, the term “associated” refers to the essentially stable combination of an agent described herein (e.g., a protein, nucleic acid molecule, vector, etc.) with one or more molecules of a carrier (e.g., one or more lipids of a lipid-based carrier, e.g., an LNP, liposome, lipoplex, and/or nanoliposome) into larger complexes or assemblies without covalent binding. In this context, the term “encapsulation” refers to the incorporation of an agent described herein (e.g., a protein, nucleic acid molecule, vector, etc.,) is entirely contained within the interior space of the carrier (e.g., the lipid-based carrier, e.g., the LNP, liposome, lipoplex, and/or nanoliposome).

Exemplary carriers are further described herein (see, e.g., § 5.15.3).

### 5.15.2 Carriers Conjugated to Immunomodulatory Fusion Proteins

In some embodiments, a fusion protein described herein, a nucleic acid molecule described herein (e.g., encoding a fusion protein described herein), a vector described herein (e.g., comprising a nucleic acid molecule (e.g., described herein) encoding a fusion protein described herein), or a conjugate described herein, is conjugated to a carrier described herein (e.g., to target the carrier (e.g., within a human subject)).

As, such, the disclosure provides, inter alia, carriers conjugated to any one of more of a fusion protein described herein, a nucleic acid molecule described herein (e.g., encoding a fusion protein described herein), a vector described herein (e.g., comprising a nucleic acid molecule (e.g., described herein) encoding a fusion protein described herein), or a conjugate described herein. In one aspect, provided herein are carriers conjugated to a fusion protein described herein. As described above, the conjugation of a fusion protein described herein to a carrier can be utilized to generate a targeted carrier (e.g., for targeting the carrier and the associated/encapsulated payload to a specific part of the body, e.g., specific tissue, cell, organ, etc.).

Exemplary carriers are further described herein (see, e.g., § 5.15.3).

### 5.15.3 Lipid Based Carriers/Lipid Nanoformulations

In some embodiments, an agent described herein (e.g., a protein, nucleic acid molecule, vector, etc.) is encapsulated or associated with one or more lipids (e.g., cationic lipids and/or neutral lipids), thereby forming lipid-based carriers such as lipid nanoparticles (LNPs), liposomes, lipoplexes, or nanoliposomes.

In some embodiments, an agent described herein (e.g., a protein, nucleic acid molecule, vector, etc.) is encapsulated in one or more lipids (e.g., cationic lipids and/or neutral lipids), thereby forming lipid-based carriers such as lipid nanoparticles (LNPs), liposomes, lipoplexes, or nanoliposomes. In some embodiments, an agent described herein (e.g., a protein, nucleic acid molecule, vector, etc.) is associated with one or more lipids (e.g., cationic lipids and/or neutral lipids), thereby forming lipid-based carriers such as lipid nanoparticles (LNPs), liposomes, lipoplexes, or nanoliposomes. In some embodiments, an agent described herein (e.g., a protein, nucleic acid molecule, vector, etc.) is encapsulated in LNPs (e.g., as described herein).

The agent (e.g., the protein, nucleic acid molecule, vector, etc.) may be completely or partially located in the interior space of the LNPs, liposomes, lipoplexes, and/or nanoliposomes, within the lipid layer/membrane, or associated with the exterior surface of the lipid layer/membrane. One purpose of incorporating an agent (e.g., a protein, nucleic acid molecule, vector, etc.) into LNPs, liposomes, lipoplexes, and/or nanoliposomes is to protect the agent from an environment which may contain enzymes or chemicals or conditions that degrade the agent from molecules or conditions that cause the rapid excretion of the agent. Moreover, incorporating an agent (e.g., a protein, nucleic acid molecule, vector, etc.) into LNPs, liposomes, lipoplexes, and/or nanoliposomes may promote the uptake of the agent, and hence, may enhance the therapeutic effect of the agent. Accordingly, incorporating an agent described herein (e.g., a protein, nucleic acid molecule, vector, etc.) into LNPs, liposomes, lipoplexes, and/or nanoliposomes may be particularly suitable for a pharmaceutical composition described herein, e.g., for intramuscular and/or intradermal administration.

In some embodiments, an agent described herein (e.g., a protein, nucleic acid molecule, vector, etc.) is formulated into a lipid-based carrier (or lipid nanoformulation). In some embodiments, the lipid-based carrier (or lipid nanoformulation) is a liposome or a lipid nanoparticle (LNP). In one embodiment, the lipid-based carrier is an LNP.

In some embodiments, an agent described herein (e.g., a protein, nucleic acid molecule, vector, etc.) is conjugated to a lipid-based carrier (e.g., described herein) (e.g., an LNP) (e.g., forming targeted lipid-based carriers (targeted LNPs)).

In some embodiments, the lipid-based carrier (or lipid nanoformulation) comprises a cationic lipid (e.g., an ionizable lipid), a non-cationic lipid (e.g., phospholipid), a structural lipid (e.g., cholesterol), and a PEG-modified lipid. In some embodiments, the lipid-based carrier (or lipid nanoformulation) contains one or more an agent described herein (e.g., a protein, nucleic acid molecule, vector, etc.), or a pharmaceutically acceptable salt thereof.

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to the one or more agent described herein (e.g., a protein, nucleic acid molecule, vector, etc.), the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

### 5.15.3.1 Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 30-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, the entire contents of which are incorporated by reference herein for all purposes. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, the entire contents of which are incorporated by reference herein for all purposes.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

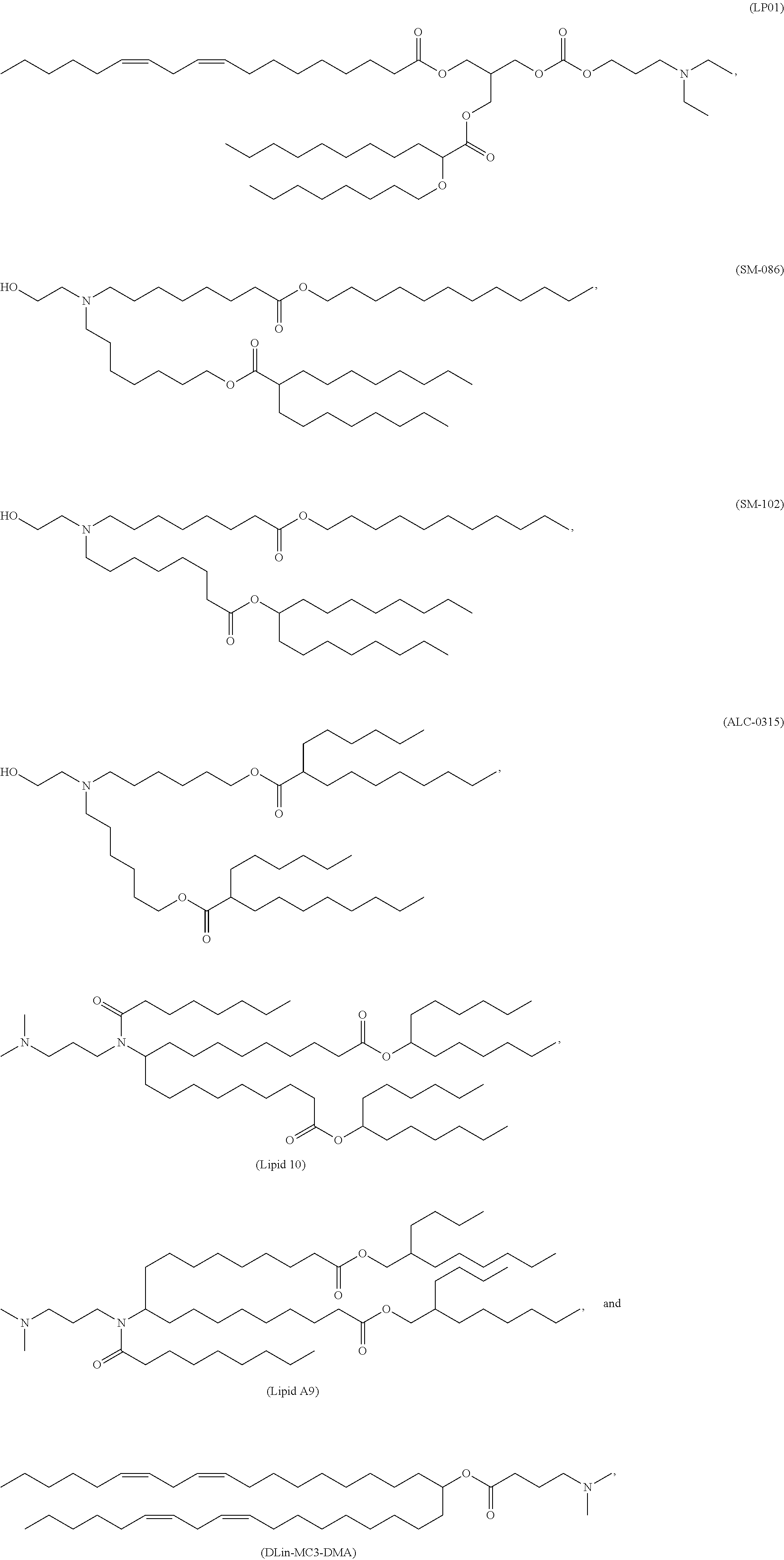

(see WO2017004143A1, the entire contents of which is incorporated herein by reference for all purposes).

In some embodiments, the lipid-based carrier (or lipid nanoformulation) further comprises one or more compounds described by WO 2021/113777 (e.g., a lipid of Formula (3) such as a lipid of Table 3 of WO 2021/113777), the entire contents of which are incorporated by reference herein for all purposes.

In one embodiment, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (the entire contents of which are incorporated by reference herein for all purposes).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; 111-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C12-200 of WO 2010/053572; 7C1 of Dahlman et al (2017); 304-013 or 503-013 of Whitehead et al; TS-P4C2 of U 59,708,628; I of WO 2020/106946; I of WO 2020/106946; (1), (2), (3), or (4) of WO 2021/113777; and any one of Tables 1-16 of WO 2021/113777, the entire contents of each of which are incorporated by reference herein for all purposes.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, the entire contents of each of which are incorporated by reference herein for all purposes.

### 5.15.3.2 Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, the entire contents of which are incorporated by reference herein for all purposes. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US2018/0028664, the entire contents of each of which are incorporated by reference herein for all purposes.

In one embodiment, the lipid-based carrier (or lipid nanoformulation) further comprises one or more non-cationic lipid that is oleic acid or a compound of Formula I, II, or IV of US2018/0028664, the entire contents of which are incorporated by reference herein for all purposes.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

### 5.15.3.3 Structural Lipids

The lipid-based carrier (or lipid nanoformulation) described herein may further comprise one or more structural lipids. As used herein, the term “structural lipid” refers to sterols (e.g., cholesterol) and also to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO2009/127060 or US2010/0130588, the entire contents of each of which are incorporated by reference herein for all purposes. Additional exemplary sterols include phytosterols, including those described in Eygeris et al. (2020), Nano Lett. 2020; 20(6):4543-4549, the entire contents of which are incorporated by reference herein for all purposes.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, the entire contents of each of which are incorporated by reference herein for all purposes.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

### 5.15.3.4 Polymers and Polyethylene Glycol (PEG)—Lipids

In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid. Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-5000](mPEG 5000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO2019/217941, the entire contents of which are incorporated by reference herein for all purposes). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (the entire contents of which are incorporated by reference herein for all purposes), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, the entire contents of each of which are incorporated by reference herein for all purposes.

In some embodiments, the PEG-lipid is a compound of Formula III, III-a-1, III-a-2, III-b-1, III-b-2, or V of US 2018/0028664, which is incorporated herein by reference in its entirety. In some embodiments, the PEG-lipid is of Formula II of US 2015/0376115 or US 2016/0376224, the entire contents of each of which are incorporated by reference herein for all purposes. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

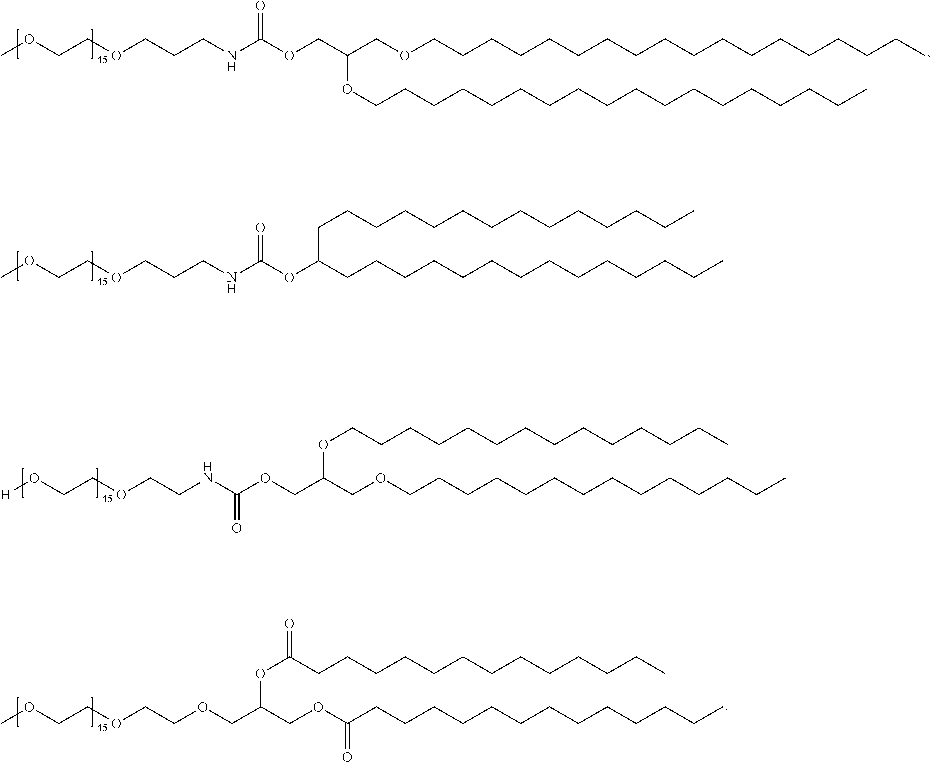

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, the entire contents of which are incorporated by reference herein for all purposes.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol % or 2-5 mol % of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), the entire contents of each of which are incorporated by reference herein for all purposes.

### 5.15.3.5 Percentages of Lipid Nanoformulation Components

In some embodiments, the lipid-based carrier (or lipid nanoformulation) comprises a non-cationic lipid (e.g., a phospholipid), a sterol, a neutral lipid, and optionally conjugated lipid (e.g., a PEGylated lipid) that inhibits aggregation of particles. The amounts of these components can be varied independently and to achieve desired properties. For example, in some embodiments, the ionizable lipid including the lipid compounds described herein is present in an amount from about 20 mol % to about 500 mol % (e.g., 20-90 mol %, 20-80 mol %, 20-70 mol %, 25-500 mol %, 30-70 mol %, 30-60 mol %, 30-40 mol %, 40-50 mol %, or 50-90 mol %) of the total lipid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid components, a conjugated lipid (e.g., a PEGylated lipid) in an amount from about 0.5 mol % to about 20 mol % (e.g., 1-10 mol % or 5-10%) of the total lipid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid components, provided that the total mol % of the lipid component does not exceed 500%.

In some embodiments, the lipid-based carrier (or lipid nanoformulation) comprises about 25-500 mol % of the ionizable lipid including the lipid compounds described herein, about 0-50 mol % phospholipid, about 0-50 mol % sterol, and about 0-10 mol % PEGylated lipid.

In some embodiments, the lipid-based carrier comprises a lipid nanoparticle, wherein the lipid nanoparticle comprises about 25-500 mol % of the ionizable lipid including the lipid compounds described herein, about 0-50 mol % phospholipid, about 0-50 mol % sterol, and about 0-10 mol % PEGylated lipid. In some embodiments, the encapsulation efficiency of the agent may be at least 70%.

In one embodiment, the lipid-based carrier (or lipid nanoformulation) comprises about 25-500 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier comprises a lipid nanoparticle, wherein the lipid nanoparticle comprises about 25-500 mol % of the ionizable lipid including the lipid compounds described herein; about 0-40 mol % phospholipid (e.g., DSPC), about 0-50 mol % sterol (e.g., cholesterol), and about 0-10 mol % PEGylated lipid. In some embodiments, the encapsulation efficiency of an agent described herein may be at least 70%.

In some embodiments, the lipid-based carrier (or lipid nanoformulation) comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, the lipid-based carrier comprises a lipid nanoparticle, wherein the lipid nanoparticle comprises about 30-60 mol % (e.g., about 35-55 mol %, or about 40-50 mol %) of the ionizable lipid including the lipid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid. In some embodiments, the encapsulation efficiency of an agent described herein may be at least 70%.

In some embodiments, molar ratios of ionizable lipid/sterol/phospholipid (or another structural lipid)/PEG-lipid/additional components is varied in the following ranges: ionizable lipid (25-500%); phospholipid (DSPC) (0-40%); sterol (0-50%); and PEG lipid (0-5%).

In some embodiments, the lipid-based carrier comprises a lipid nanoparticle, wherein the lipid nanoparticle comprises molar ratios of ionizable lipid/sterol/phospholipid (or another structural lipid)/PEG-lipid/additional components in the following ranges: ionizable lipid (25-500%); phospholipid (DSPC) (0-40%); sterol (0-50%); and PEG lipid (0-5%). In some embodiments, the encapsulation efficiency of an agent described herein may be at least 70%.

In some embodiments, the lipid-based carrier (or lipid nanoformulation) comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid).

In some embodiments, the lipid-based carrier comprises a lipid nanoparticle, wherein the lipid nanoparticle comprises, by mol % or wt % of the total lipid components, 50-75% ionizable lipid (including the lipid compound as described herein), 20-40% sterol (e.g., cholesterol or derivative), 0 to 10% non-cationic-lipid, and 1-10% conjugated lipid (e.g., the PEGylated lipid). In some embodiments, the encapsulation efficiency of an agent described herein may be at least 70%.

In some embodiments, the lipid-based carrier (or lipid nanoformulation) comprises (i) a cationic lipid comprising from 50 mol % to 65 mol % of the total lipid present in the lipid-based carrier; (ii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iii) a conjugated lipid comprising 0.5 mol % to 2 mol % of the total lipid present in the particle.

In some embodiments, the lipid-based carrier (or lipid nanoformulation) comprises (i) an agent described herein (e.g., a protein, nucleic acid molecule, vector, etc.); (ii) a cationic lipid comprising from 50 mol % to 65 mol % of the total lipid present in the lipid-based carrier; (iii) a non-cationic lipid comprising a mixture of a phospholipid and a cholesterol derivative thereof, wherein the phospholipid comprises from 3 mol % to 15 mol % of the total lipid present in the lipid-based carrier and the cholesterol or derivative thereof comprises from 30 mol % to 40 mol % of the total lipid present in the lipid-based carrier; and (iv) a conjugated lipid comprising 0.5 mol % to 2 mol % of the total lipid present in the particle.

In some embodiments, the lipid-based carrier (or lipid nanoformulation) comprises (i) a cationic lipid comprising from 50 mol % to 85 mol % of the total lipid present in the lipid-based carrier; (ii) a non-cationic lipid comprising from 13 mol % to 49.5 mol % of the total lipid present in the lipid-based carrier; and (iii) a conjugated lipid comprising from 0.5 mol % to 2 mol % of the total lipid present in the lipid-based carrier.

In some embodiments, the lipid-based carrier (or lipid nanoformulation) comprises (i) an agent described herein (e.g., a protein, nucleic acid molecule, vector, etc.); (ii) a cationic lipid comprising from 50 mol % to 85 mol % of the total lipid present in the lipid-based carrier; (iii) a non-cationic lipid comprising from 13 mol % to 49.5 mol % of the total lipid present in the lipid-based carrier; and (iv) a conjugated lipid comprising from 0.5 mol % to 2 mol % of the total lipid present in the lipid-based carrier.

In some embodiments, the phospholipid component in the mixture may be present from 2 mol % to 20 mol %, from 2 mol % to 15 mol %, from 2 mol % to 12 mol %, from 4 mol % to 15 mol %, from 4 mol % to 10 mol %, from 5 mol % to 10 mol %, (or any fraction of these ranges) of the total lipid components. In some embodiments, the lipid-based carrier (or lipid nanoformulation) is phospholipid-free.

In some embodiments, the sterol component (e.g. cholesterol or derivative) in the mixture may comprise from 25 mol % to 45 mol %, from 25 mol % to 40 mol %, from 25 mol % to 35 mol %, from 25 mol % to 30 mol %, from 30 mol % to 45 mol %, from 30 mol % to 40 mol %, from 30 mol % to 35 mol %, from 35 mol % to 40 mol %, from 27 mol % to 37 mol %, or from 27 mol % to 35 mol % (or any fraction of these ranges) of the total lipid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid components.

The ratio of total lipid components to the agent can be varied as desired. For example, the total lipid components to the agent (mass or weight) ratio can be from about 10:1 to about 30:1. In some embodiments, the total lipid components to the agent ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid components and the agent can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation's) overall lipid content can range from about 5 mg/ml to about 30 mg/mL. Nitrogen:phosphate ratios (N:P ratio) is evaluated at values between 0.1 and 500.

The efficiency of encapsulation of an agent described herein (e.g., a protein, nucleic acid molecule, vector, etc.), describes the amount of the agent that is encapsulated or otherwise associated with a lipid nanoformulation (e.g., liposome or LNP) after preparation, relative to the initial amount provided. The encapsulation efficiency is desirably high (e.g., at least 70%, 80%. 90%, 95%, close to 500%). The encapsulation efficiency may be measured, for example, by comparing the amount of the agent in a solution containing the liposome or LNP before and after breaking up the liposome or LNP with one or more organic solvents or detergents. An anion exchange resin may be used to measure the amount of free the agent in a solution. Fluorescence may be used to measure the amount of free the agent in a solution. For the lipid-based carrier (or lipid nanoformulation) described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 500%. In some embodiments, the encapsulation efficiency may be at least 70%. In some embodiments, the encapsulation efficiency may be at least 80%. In some embodiments, the encapsulation efficiency may be at least 90%. In some embodiments, the encapsulation efficiency may be at least 95%.

### 5.16 Pharmaceutical Compositions

In one aspect, provided herein are pharmaceutical compositions comprising any one or more of an agent described herein, including, e.g., a fusion protein described herein, a nucleic acid molecule described herein (e.g., encoding a fusion protein described herein), or a vector described herein (e.g., comprising a nucleic acid molecule (e.g., described herein) encoding a fusion protein described herein), a cell described herein, or a carrier described herein, and a pharmaceutically acceptable excipient (see, e.g., Remington's Pharmaceutical Sciences (1990) Mack Publishing Co., Easton, PA, the entire contents of which is incorporated by reference herein for all purposes).

Also provided herein are pharmaceutical compositions comprising an agent described herein, including, e.g., a fusion protein described herein, a nucleic acid molecule described herein (e.g., encoding a fusion protein described herein), or a vector described herein (e.g., comprising a nucleic acid molecule (e.g., described herein) encoding a fusion protein described herein), a cell described herein, or a carrier described herein, and a pharmaceutically acceptable excipient, wherein the pharmaceutical composition lacks a predetermined threshold amount or a detectable amount of a process impurity or contaminant, e.g., lacks a predetermined threshold amount or a detectable amount of a process-related impurity such as host cell proteins, host cell DNA, or a cell culture component (e.g., inducers, antibiotics, or media components); a product-related impurity (e.g., precursors, fragments, aggregates, degradation products); or a contaminant, e.g., endotoxin, bacteria, viral contaminant.

In one aspect, also provided herein are methods of making pharmaceutical compositions described herein comprising providing an agent described herein, including, e.g., a fusion protein described herein, a nucleic acid molecule described herein (e.g., encoding a fusion protein described herein), or a vector described herein e.g., comprising a nucleic acid molecule (e.g., described herein) encoding a fusion protein described herein), a cell described herein, or a carrier described herein, and formulating it into a pharmaceutically acceptable composition by the addition of one or more pharmaceutically acceptable excipient.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

A pharmaceutical composition may be formulated for any route of administration to a subject. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.17 Combination Regimens

In one aspect, provided herein are combination regimens comprising at least two immunomodulatory proteins (e.g., described herein) (or one or more nucleic acid molecule encoding the same), wherein each of the at least two immunomodulatory proteins comprises an IL-10 binding protein described herein, a TL1A binding protein described herein, a TNFα binding protein described herein, a CD30L binding protein described herein, or a fusion protein described herein (e.g., a fusion protein comprising at least two of the foregoing).

The at least two immunomodulatory proteins of the combination regimen may or may not be combined in a single dosage form. In some embodiments, the at least two proteins of the combination regimen are not combined in a single dosage form. In some embodiments, at least two proteins of the combination regimen are combined in a single dosage form.

The at least two proteins of the combination regimen may be formulated in separate formulations or in single formulation. In some embodiments, the at least two proteins of the combination regimen are formulated in separate formulations. In some embodiments, the at least two proteins of the combination regimen are formulated in a single formulation.

The at least two proteins of the combination regimen may be formulated in separate pharmaceutical formulations or in single pharmaceutical formulation. In some embodiments, the at least two proteins of the combination regimen are formulated in separate pharmaceutical formulations. In some embodiments, the at least two proteins of the combination regimen are formulated in a single pharmaceutical formulation.

The combination regimens described herein can be utilized, e.g., in any of the combination compositions described herein, see e.g., § 5.18; in any of the pharmaceutical compositions described herein see e.g., § 5.16; in any of the methods of use described herein, see e.g., § 5.19; in any of the kits described herein see e.g., § 5.20.

In some embodiments, the combination regimen comprises a first IL-10 binding protein described herein and a second IL-10 binding protein described herein (or one or more nucleic acid molecule encoding each of the foregoing). In some embodiments, the first and second IL-10 binding proteins are the same. In some embodiments, the first and second IL-10 binding proteins are different. In some embodiments, the combination regimen comprises an IL-10 binding protein described herein and a TL1A binding protein described herein (or one or more nucleic acid molecule encoding each of the foregoing). In some embodiments, the combination regimen comprises an IL-10 binding protein described herein and a TNFα binding protein described herein (or one or more nucleic acid molecule encoding each of the foregoing). In some embodiments, the combination regimen comprises an IL-10 binding protein described herein and a CD30L binding protein described herein (or one or more nucleic acid molecule encoding each of the foregoing). In some embodiments, the combination regimen comprises an IL-10 binding protein described herein and a fusion protein described herein (or one or more nucleic acid molecule encoding each of the foregoing).

In some embodiments, the combination regimen comprises a first TL1A binding protein described herein and a second TL1A binding protein described herein (or one or more nucleic acid molecule encoding each of the foregoing). In some embodiments, the first and second TL1A binding proteins are the same. In some embodiments, the first and second TL1A binding proteins are different. In some embodiments, the combination regimen comprises a TL1A binding protein described herein and a TNFα binding protein described herein (or one or more nucleic acid molecule encoding each of the foregoing). In some embodiments, the combination regimen comprises a TL1A binding protein described herein and a CD30L binding protein described herein (or one or more nucleic acid molecule encoding each of the foregoing). In some embodiments, the combination regimen comprises a TL1A binding protein described herein and a fusion protein described herein (or one or more nucleic acid molecule encoding each of the foregoing).

In some embodiments, the combination regimen comprises a first TNFα binding protein described herein and a second TNFα binding protein described herein (or one or more nucleic acid molecule encoding each of the foregoing). In some embodiments, the first and second TNFα binding proteins are the same. In some embodiments, the first and second TNFα binding proteins are different. In some embodiments, the combination regimen comprises a TNFα binding protein described herein and a CD30L binding protein described herein (or one or more nucleic acid molecule encoding each of the foregoing). In some embodiments, the combination regimen comprises a TNFα binding protein described herein and a fusion protein described herein (or one or more nucleic acid molecule encoding each of the foregoing).

In some embodiments, the combination regimen comprises a first CD30L binding protein described herein and a second CD30L binding protein described herein (or one or more nucleic acid molecule encoding each of the foregoing). In some embodiments, the first and second CD30L binding proteins are the same. In some embodiments, the first and second CD30L binding proteins are different. In some embodiments, the combination regimen comprises a CD30L binding protein described herein and a fusion protein described herein (or one or more nucleic acid molecule encoding each of the foregoing).

In some embodiments, the combination regimen comprises a first fusion protein described herein and a second fusion protein described herein (or one or more nucleic acid molecule encoding each of the foregoing). In some embodiments, the first and second fusion proteins are the same. In some embodiments, the first and second fusion proteins are different.

### 5.18 Combination Compositions

In one aspect, provided herein are combination compositions (e.g., pharmaceutical compositions) comprising at least two immunomodulatory proteins (e.g., described herein) (or one or more nucleic acid molecule encoding the same), wherein each of the at least two immunomodulatory proteins comprises an IL-10 binding protein described herein, a TL1A binding protein described herein, a TNFα binding protein described herein, a CD30L binding protein described herein, or a fusion protein described herein (e.g., a fusion protein comprising at least two of the foregoing).

The combination compositions described herein can be utilized, e.g., in any of the combination regimens described herein, see e.g., § 5.17; in any of the pharmaceutical compositions described herein see e.g., § 5.16; in any of the methods of use described herein, see e.g., § 5.19; in any of the kits described herein see e.g., § 5.20.

In some embodiments, the combination composition comprises a first IL-10 binding protein described herein and a second IL-10 binding protein described herein (or one or more nucleic acid molecule encoding each of the foregoing). In some embodiments, the first and second IL-10 binding proteins are the same. In some embodiments, the first and second IL-10 binding proteins are different. In some embodiments, the combination composition comprises an IL-10 binding protein described herein and a TL1A binding protein described herein (or one or more nucleic acid molecule encoding each of the foregoing). In some embodiments, the combination composition comprises an IL-10 binding protein described herein and a TNFα binding protein described herein (or one or more nucleic acid molecule encoding each of the foregoing). In some embodiments, the combination composition comprises an IL-10 binding protein described herein and a CD30L binding protein described herein (or one or more nucleic acid molecule encoding each of the foregoing). In some embodiments, the combination composition comprises an IL-10 binding protein described herein and a fusion protein described herein (or one or more nucleic acid molecule encoding each of the foregoing).

In some embodiments, the combination composition comprises a first TL1A binding protein described herein and a second TL1A binding protein described herein (or one or more nucleic acid molecule encoding each of the foregoing). In some embodiments, the first and second TL1A binding proteins are the same. In some embodiments, the first and second TL1A binding proteins are different. In some embodiments, the combination composition comprises a TL1A binding protein described herein and a TNFα binding protein described herein (or one or more nucleic acid molecule encoding each of the foregoing). In some embodiments, the combination composition comprises a TL1A binding protein described herein and a CD30L binding protein described herein (or one or more nucleic acid molecule encoding each of the foregoing). In some embodiments, the combination composition comprises a TL1A binding protein described herein and a fusion protein described herein (or one or more nucleic acid molecule encoding each of the foregoing).

In some embodiments, the combination composition comprises a first TNFα binding protein described herein and a second TNFα binding protein described herein (or one or more nucleic acid molecule encoding each of the foregoing). In some embodiments, the first and second TNFα binding proteins are the same. In some embodiments, the first and second TNFα binding proteins are different. In some embodiments, the combination composition comprises a TNFα binding protein described herein and a CD30L binding protein described herein (or one or more nucleic acid molecule encoding each of the foregoing). In some embodiments, the combination composition comprises a TNFα binding protein described herein and a fusion protein described herein (or one or more nucleic acid molecule encoding each of the foregoing).

In some embodiments, the combination composition comprises a first CD30L binding protein described herein and a second CD30L binding protein described herein (or one or more nucleic acid molecule encoding each of the foregoing). In some embodiments, the first and second CD30L binding proteins are the same. In some embodiments, the first and second CD30L binding proteins are different. In some embodiments, the combination composition comprises a CD30L binding protein described herein and a fusion protein described herein (or one or more nucleic acid molecule encoding each of the foregoing).

In some embodiments, the combination composition comprises a first fusion protein described herein and a second fusion protein described herein (or one or more nucleic acid molecule encoding each of the foregoing). In some embodiments, the first and second fusion proteins are the same. In some embodiments, the first and second fusion proteins are different.

### 5.19 Methods of Use

Provided herein are various methods of utilizing any one or more agent described herein, including e.g., a fusion protein described herein, a nucleic acid molecule described herein (e.g., encoding a fusion protein described herein), a vector described herein (e.g., comprising a nucleic acid molecule (e.g., described herein) encoding a fusion protein described herein), a conjugate described herein, a cell described herein, a carrier described herein, a pharmaceutical composition described herein, a combination regimen described herein, or a combination composition described herein. Exemplary subjects include mammals, e.g., humans, non-human mammals, e.g., non-human primates. In some embodiments, the subject is a human.

The dosage of an agent described herein (e.g., a fusion protein described herein, a nucleic acid molecule described herein (e.g., encoding a fusion protein described herein), a vector described herein (e.g., comprising a nucleic acid molecule (e.g., described herein) encoding a fusion protein described herein), a conjugate described herein, a cell described herein, a carrier described herein, a pharmaceutical composition described herein, a combination regimen described herein, or a combination composition described herein) to be administered to a subject in accordance with any of the methods described herein can be determined in accordance with standard techniques known to those of ordinary skill in the art, including the route of administration, the age and weight of the subject, and the type (if any) adjuvant is used.

### 5.19.1 Methods of Delivery

Provided herein are methods of delivering a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition described herein, a combination regimen described herein, or a combination composition described herein to a subject, the method comprising administering to the subject the fusion protein, the nucleic acid molecule, the vector described herein, the conjugate, the cell, the carrier, or the pharmaceutical composition, to thereby deliver the fusion protein, the nucleic acid molecule, the vector described herein, the conjugate, the cell, the carrier, the pharmaceutical composition, the combination regimen described herein, or the combination composition described herein to the subject.

In some embodiments, the fusion protein, the nucleic acid molecule, the vector described herein, the conjugate, the cell, the carrier, or the pharmaceutical composition is administered to the subject in an amount and for a time sufficient to deliver the fusion protein, the nucleic acid molecule, the vector described herein, the conjugate, the cell, the carrier, the pharmaceutical composition, the combination regimen described herein, or the combination composition described herein to the subject.

### 5.19.2 Methods of Inhibiting or Reducing (e.g., Preventing) Receptor-Ligand Interactions

Provided herein are methods of inhibiting or reducing (e.g., preventing) binding of a receptor to a cognate ligand, the method comprising administering to the subject a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, or a pharmaceutical composition described herein; to thereby inhibit or reduce (e.g., prevent) binding of a receptor to a cognate ligand in the subject. In some embodiments, the fusion protein, the nucleic acid molecule, the vector described herein, the conjugate, the cell, the carrier, or the pharmaceutical composition is administered to the subject in an amount and for a time sufficient to inhibit or reduce (e.g., prevent) binding of a receptor to a cognate ligand in the subject.

In some embodiments, receptor and cognate ligand is hTL1A and hDR3. In some embodiments, receptor and cognate ligand is hTNFα and hTNFR1 and/or hTNFR2.

Provided herein is a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, or a pharmaceutical composition described herein for use in a method of inhibiting or reducing (e.g., preventing) binding of a receptor to a cognate ligand in a subject in need thereof, the method comprising administering to the subject the fusion protein, the nucleic acid molecule, the vector described herein, the conjugate, the cell, the carrier, or the pharmaceutical composition, to thereby inhibit or reduce (e.g., prevent) binding of a receptor to a cognate ligand in the subject.

Provided herein are uses a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, or a pharmaceutical composition described herein for the manufacture of a medicament for of inhibiting or reducing (e.g., preventing) binding of a receptor to a cognate ligand in a subject in need thereof.

### 5.19.3 Methods of Inhibiting or Reducing (e.g., Preventing) Binding of a Plurality of Respective Receptor Ligand Interactions

Provided herein are methods of inhibiting or reducing (e.g., preventing) binding of a plurality of respective receptor ligand interactions, the method comprising administering to the subject a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition, a combination regimen described herein, or a combination composition described herein described herein; to thereby inhibit or reduce (e.g., prevent) a plurality of respective receptor ligand interactions in the subject. In some embodiments, the fusion protein, the nucleic acid molecule, the vector described herein, the conjugate, the cell, the carrier, the pharmaceutical composition, the combination regimen described herein, or the combination composition described herein is administered to the subject the in an amount and for a time sufficient to inhibit or reduce (e.g., prevent) a plurality of respective receptor ligand interactions in the subject.

In some embodiments, the plurality comprises the binding of at least two of: hTL1A to hDR3, hTNFα to hTNFR1 and/or hTNFR2, and/or hCD30L to hCD30. In some embodiments, the plurality comprises the binding of hTL1A to hDR3 and hTNFα to hTNFR1 and/or hTNFR2. In some embodiments, the plurality comprises the binding of hTL1A to hDR3 and hCD30L to hCD30. In some embodiments, the plurality comprises the binding of hTNFα to hTNFR1 and/or hTNFR2 and hTL1A to hDR3. In some embodiments, the plurality comprises the binding of hTNFα to hTNFR1 and/or hTNFR2 and hLTα to hTNFR1 and/or hTNFR2. In some embodiments, the plurality comprises the binding of hTNFα to hTNFR1 and/or hTNFR2 and hCD30L to hCD30. In some embodiments, the plurality comprises the binding of hCD30L to hCD30 and hTL1A to hDR3. In some embodiments, the plurality comprises the binding of hCD30L to hCD30 and hTNFα to hTNFR1 and/or hTNFR2. In some embodiments, the plurality comprises the binding of hTL1A to hDR3, hTNFα to hTNFR1 and/or hTNFR2, and hCD30L to hCD30.

Provided herein is a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition, a combination regimen described herein, or a combination composition described herein described herein for use in a method of inhibiting or reducing (e.g., preventing) binding of a plurality of respective receptor ligand interactions in a subject in a subject in need thereof, the method comprising administering to the subject the fusion protein, the nucleic acid molecule, the vector described herein, the conjugate, the cell, the carrier, the pharmaceutical composition, the combination regimen described herein, or the combination composition described herein, to thereby inhibit or reduce (e.g., prevent) binding of a plurality of respective receptor ligand interactions in a subject in the subject.

Provided herein are uses a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition described herein, a combination regimen described herein, or a combination composition described herein for the manufacture of a medicament for inhibiting or reducing (e.g., preventing) binding of a plurality of respective receptor ligand interactions in a subject in a subject in need thereof.

### 5.19.4 Methods of Inhibiting or Reducing (e.g., Preventing) Signaling Mediated by a Receptor Ligand Interaction

Provided herein are methods of inhibiting or reducing (e.g., preventing) signaling mediated by the binding of a receptor to its cognate ligand in a subject in need thereof, the method comprising administering to the subject a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, or a pharmaceutical composition described herein; to thereby inhibit or reduce (e.g., prevent) signaling mediated by the binding of the receptor to its cognate ligand in the subject in need thereof. In some embodiments, the fusion protein, the nucleic acid molecule, the vector described herein, the conjugate, the cell, the carrier, or the pharmaceutical composition is administered to the subject the in an amount and for a time sufficient to inhibit or reduce (e.g., prevent) signaling mediated by the binding of the receptor to its cognate ligand in the subject in need thereof.

In some embodiments, receptor and cognate ligand is hTL1A and hDR3. In some embodiments, receptor and cognate ligand is hTNFα and hTNFR1 and/or hTNFR2.

Provided herein is a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, or a pharmaceutical composition described herein for use in a method of inhibiting or reducing (e.g., preventing) signaling mediated by a plurality of respective receptor ligand interactions in a subject in need thereof, the method comprising administering to the subject the fusion protein, the nucleic acid molecule, the vector described herein, the conjugate, the cell, the carrier, or the pharmaceutical composition, to thereby inhibit or reduce (e.g., prevent) signaling mediated by a plurality of respective receptor ligand interactions in the subject.

Provided herein are uses of a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, or a pharmaceutical composition described herein for the manufacture of a medicament for inhibiting or reducing (e.g., preventing) signaling mediated by a plurality of respective receptor ligand interactions in a subject in need thereof.

### 5.19.5 Methods of Inhibiting or Reducing (e.g., Preventing) Signaling Mediated by a Plurality of Respective Receptor Ligand Interactions

Provided herein are methods of inhibiting or reducing (e.g., preventing) signaling mediated by a plurality of respective receptor ligand interactions in a subject in need thereof, the method comprising administering to the subject a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition described herein, a combination regimen described herein, or a combination composition described herein; to thereby inhibit or reduce (e.g., prevent) signaling mediated by a plurality of respective receptor ligand interactions in the subject. In some embodiments, the fusion protein, the nucleic acid molecule, the vector described herein, the conjugate, the cell, the carrier, the pharmaceutical composition, the combination regimen described herein, or the combination composition described herein is administered to the subject the in an amount and for a time sufficient to inhibit or reduce (e.g., prevent) signaling mediated by a plurality of respective receptor ligand interactions in the subject.

In some embodiments, the plurality comprises the signaling mediated by the binding of at least two of: hTL1A to hDR3, hTNFα to hTNFR1 and/or hTNFR2, and/or hCD30L to hCD30. In some embodiments, the plurality comprises the signaling mediated by the binding of hTL1A to hDR3 and hTNFα to hTNFR1 and/or hTNFR2. In some embodiments, the plurality comprises the signaling mediated by the binding of hTL1A to hDR3 and hCD30L to hCD30. In some embodiments, the plurality comprises the signaling mediated by the binding of hTNFα to hTNFR1 and/or hTNFR2 and hTL1A to hDR3. In some embodiments, the plurality comprises the signaling mediated by the binding of hTNFα to hTNFR1 and/or hTNFR2 and hLTα to hTNFR1 and/or hTNFR2. In some embodiments, the plurality comprises the signaling mediated by the binding of hTNFα to hTNFR1 and/or hTNFR2 and hCD30L to hCD30. In some embodiments, the plurality comprises the signaling mediated by the binding of hCD30L to hCD30 and hTL1A to hDR3. In some embodiments, the plurality comprises the signaling mediated by the binding of hCD30L to hCD30 and hTNFα to hTNFR1 and/or hTNFR2. In some embodiments, the plurality comprises the signaling mediated by the binding of hTL1A to hDR3, hTNFα to hTNFR1 and/or hTNFR2, and hCD30L to hCD30.

Provided herein is a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition, a combination regimen described herein, or a combination composition described herein described herein for use in a method of inhibiting or reducing (e.g., preventing) signaling mediated by a plurality of respective receptor ligand interactions in a subject in need thereof, the method comprising administering to the subject the fusion protein, the nucleic acid molecule, the vector described herein, the conjugate, the cell, the carrier, the pharmaceutical composition, the combination regimen described herein, or the combination composition described herein, to thereby inhibit or reduce (e.g., prevent) signaling mediated by a plurality of respective receptor ligand interactions in the subject.

Provided herein are uses of a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition, a combination regimen described herein, or a combination composition described herein described herein for the manufacture of a medicament for inhibiting or reducing (e.g., preventing) signaling mediated by a plurality of respective receptor ligand interactions in a subject in need thereof.

### 5.19.6 Methods of Suppressing or Reducing (e.g., Preventing) a Pro-Inflammatory Immune Response

Provided herein are methods of suppressing or reducing (e.g., preventing) a pro-inflammatory immune response in a subject in need thereof, the method comprising administering to the subject a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition, a combination regimen described herein, or a combination composition described herein described herein, to thereby suppress or reduce (e.g., prevent) a pro-inflammatory immune response in the subject. In some embodiments, the fusion protein, the nucleic acid molecule, the vector described herein, the conjugate, the cell, the carrier, the pharmaceutical composition, the combination regimen described herein, or the combination composition described herein is administered to the subject the in an amount and for a time sufficient to suppress or reduce (e.g., prevent) a pro-inflammatory immune response in the subject.

Provided herein is a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition, a combination regimen described herein, or a combination composition described herein described herein for use in a method of suppressing or reducing (e.g., preventing) a pro-inflammatory immune response in a subject in need thereof, the method comprising administering to the subject the fusion protein, the nucleic acid molecule, the vector described herein, the conjugate, the cell, the carrier, the pharmaceutical composition, the combination regimen described herein, or the combination composition described herein, to thereby suppress or reduce (e.g., prevent) a pro-inflammatory immune response in the subject.

Provided herein are uses of a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition, a combination regimen described herein, or a combination composition described herein described herein for the manufacture of a medicament for suppressing or reducing (e.g., preventing) a pro-inflammatory immune response in a subject in need thereof.

### 5.19.7 Methods of Preventing, Treating, or Ameliorating a Disease in a Subject in Need Thereof

Provided herein are methods of preventing, treating, or ameliorating a disease in a subject in a subject in need thereof, the method comprising administering to the subject a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition described herein, a combination regimen described herein, or a combination composition described herein, to thereby prevent, treat, or ameliorate the disease in the subject. In some embodiments, the fusion protein, the nucleic acid molecule, the vector described herein, the conjugate, the cell, the carrier, the pharmaceutical composition, the combination regimen described herein, or the combination composition described herein is administered to the subject the in an amount and for a time sufficient to prevent, treat, or ameliorate the disease in the subject.

In some embodiments, the disease is a pro-inflammatory disease, an autoimmune disease, or a metabolic inflammatory disease.

In some embodiments, the disease is an autoimmune disease, e.g., an inflammatory bowel disease (e.g., ulcerative colitis, Crohn's disease), rheumatoid arthritis, multiple sclerosis, psoriasis, or autoimmune hepatitis. In some embodiments, the disease is a pro-inflammatory disease, e.g., cytokine storm, arthritis (e.g., osteoarthritis), or graft-versus host disease (e.g., in the context of organ, tissue, or cell transplant). In some embodiments, the disease is an allergy, e.g., allergic asthma or a food allergy. In some embodiments, the disease is an infectious disease, e.g., a viral infection, e.g., hepatitis C. In some embodiments, the disease is a fibrotic disease, e.g., pulmonary fibrosis, liver fibrosis, pancreatitis, chronic kidney disease, cardiovascular fibrosis, or myocardial infarction. In some embodiments, the disease is an arterial disease, e.g., atherosclerosis. In some embodiments, the disease is a disease associated with pregnancy, e.g., pre-eclampsia or inflammation-driven fetal death. In some embodiments, the disease is a nervous system disease, e.g., a neurodegenerative disease (e.g., Parkinson's disease), neuropathic nerve pain, or peripheral nerve pain. In some embodiments, the disease is cancer. In some embodiments, the disease is metabolic inflammatory syndrome. In some embodiments, the disease is insulin resistance, atherosclerosis, or type 2 diabetes. In some embodiments, the subject has one or more of insulin resistance, atherosclerosis, or type 2 diabetes. In some embodiments, the metabolic inflammatory syndrome comprises one or more of insulin resistance, atherosclerosis, or type 2 diabetes. In some embodiments, the disease is a liver disease. In some embodiments, the liver disease is fatty liver, liver inflammation, nonalcoholic steatohepatitis (NASH), or nonalcoholic fatty liver disease (NAFLD).

In some embodiments, the disease is adult inflammatory bowel disease. In some embodiments, the disease is adult ulcerative colitis. In some embodiments, the disease is adult Crohn's disease.

In some embodiments, the disease is inflammatory bowel disease, Crohn's disease, ulcerative colitis, rheumatoid arthritis, psoriatic arthritis, plaque psoriasis, juvenile idiopathic arthritis, Hidradenitis suppurativa, uveitis, non-radiographic axial spondyloarthritis, ankylosing spondylitis, asthma, systemic lupus erythematosus.

In some embodiments, the disease is inflammatory bowel disease, Crohn's disease, ulcerative colitis, rheumatoid arthritis, psoriatic arthritis, ankylosing spondylitis, asthma, systemic lupus erythematosus, or fibrosis.

In some embodiments, the pro-inflammatory disease is an autoimmune disease. In some embodiments, the pro-inflammatory (e.g., autoimmune) disease comprises flares (e.g., relapsing-remitting forms of pro-inflammatory (e.g., autoimmune) diseases). In some embodiments, the agent (e.g., a fusion protein described herein) is administered to the subject prior to, at the start of, at initial signs of, or during a flare. In some embodiments, the pro-inflammatory (e.g., autoimmune) disease is multiple sclerosis, rheumatoid arthritis, inflammatory bowel disease, systemic lupus erythematosus, myasthenia gravis, or psoriasis. In some embodiments, the pro-inflammatory (e.g., autoimmune) disease is relapsing-remitting multiple sclerosis, relapsing-remitting rheumatoid arthritis, relapsing-remitting inflammatory bowel disease, relapsing-remitting systemic lupus erythematosus, relapsing-remitting myasthenia gravis, or relapsing-remitting psoriasis.

In some embodiments, the agent administered to the subject (e.g., the fusion protein described herein) comprises one or more IL-10R binding protein described herein and the disease is a pro-inflammatory disease, an autoimmune disease, or a metabolic inflammatory disease. In some embodiments, the agent administered to the subject (e.g., the fusion protein described herein) comprises one or more IL-10R binding protein described herein and the disease is an autoimmune disease, e.g., an inflammatory bowel disease (e.g., ulcerative colitis, Crohn's disease), rheumatoid arthritis, multiple sclerosis, psoriasis, or autoimmune hepatitis. In some embodiments, the agent administered to the subject (e.g., the fusion protein described herein) comprises one or more IL-10R binding protein described herein and the disease is a pro-inflammatory disease, e.g., cytokine storm, arthritis (e.g., osteoarthritis), or graft-versus host disease (e.g., in the context of organ, tissue, or cell transplant). In some embodiments, the agent administered to the subject (e.g., the fusion protein described herein) comprises one or more IL-10R binding protein described herein and the disease is an allergy, e.g., allergic asthma or a food allergy. In some embodiments, the agent administered to the subject (e.g., the fusion protein described herein) comprises one or more IL-10R binding protein described herein and the disease is an infectious disease, e.g., a viral infection, e.g., hepatitis C. In some embodiments, the agent administered to the subject (e.g., the fusion protein described herein) comprises one or more IL-10R binding protein described herein and the disease is a fibrotic disease, e.g., pulmonary fibrosis, liver fibrosis, pancreatitis, chronic kidney disease, cardiovascular fibrosis, or myocardial infarction. In some embodiments, the agent administered to the subject (e.g., the fusion protein described herein) comprises one or more IL-10R binding protein described herein and the disease is an arterial disease, e.g., atherosclerosis. In some embodiments, the agent administered to the subject (e.g., the fusion protein described herein) comprises one or more IL-10R binding protein described herein and the disease is a disease associated with pregnancy, e.g., pre-eclampsia or inflammation-driven fetal death. In some embodiments, the agent administered to the subject (e.g., the fusion protein described herein) comprises one or more IL-10R binding protein described herein and the disease is a nervous system disease, e.g., a neurodegenerative disease (e.g., Parkinson's disease), neuropathic nerve pain, or peripheral nerve pain. In some embodiments, the agent administered to the subject (e.g., the fusion protein described herein) comprises one or more IL-10R binding protein described herein and the disease is cancer. In some embodiments, the agent administered to the subject (e.g., the fusion protein described herein) comprises one or more IL-10R binding protein described herein and the disease is metabolic inflammatory syndrome. In some embodiments, the agent administered to the subject (e.g., the fusion protein described herein) comprises one or more IL-10R binding protein described herein and the disease is insulin resistance, atherosclerosis, or type 2 diabetes. In some embodiments, the agent administered to the subject (e.g., the fusion protein described herein) comprises one or more IL-10R binding protein described herein and the subject has one or more of insulin resistance, atherosclerosis, or type 2 diabetes. In some embodiments, the agent administered to the subject (e.g., the fusion protein described herein) comprises one or more IL-10R binding protein described herein and the metabolic inflammatory syndrome comprises one or more of insulin resistance, atherosclerosis, or type 2 diabetes. In some embodiments, the agent administered to the subject (e.g., the fusion protein described herein) comprises one or more IL-10R binding protein described herein and the disease is a liver disease. In some embodiments, the agent administered to the subject (e.g., the fusion protein described herein) comprises one or more IL-10R binding protein described herein and the liver disease is fatty liver, liver inflammation, nonalcoholic steatohepatitis (NASH), or nonalcoholic fatty liver disease (NAFLD).

In some embodiments, the agent administered to the subject (e.g., the fusion protein described herein) comprises one or more TNF binding protein described herein, and the disease is a pro-inflammatory disease, an autoimmune disease, or a metabolic inflammatory disease. In some embodiments, the agent administered to the subject (e.g., the fusion protein described herein) comprises one or more TNF binding protein described herein, and the disease is inflammatory bowel disease, Crohn's disease, ulcerative colitis, rheumatoid arthritis, psoriatic arthritis, plaque psoriasis, juvenile idiopathic arthritis, Hidradenitis suppurativa, uveitis, non-radiographic axial spondyloarthritis, ankylosing spondylitis, asthma, or systemic lupus erythematosus.

In some embodiments, the agent administered to the subject (e.g., the fusion protein described herein) comprises one or more TL1A binding protein described herein, and the disease is a pro-inflammatory disease, an autoimmune disease, or a metabolic inflammatory disease. In some embodiments, the agent administered to the subject (e.g., the fusion protein described herein) comprises one or more TL1A binding protein described herein and the disease is inflammatory bowel disease, Crohn's disease, ulcerative colitis, rheumatoid arthritis, psoriatic arthritis, ankylosing spondylitis, asthma, systemic lupus erythematosus, or fibrosis.

In some embodiments, the agent administered to the subject (e.g., the fusion protein described herein) comprises one or more CD30L binding protein described herein, and the disease is a pro-inflammatory disease. In some embodiments, the agent administered to the subject (e.g., the fusion protein described herein) comprises one or more CD30L binding protein described herein, and the disease is an autoimmune disease. In some embodiments, the agent administered to the subject (e.g., the fusion protein described herein) comprises one or more CD30L binding protein described herein, and the pro-inflammatory (e.g., autoimmune) disease comprises flares (e.g., relapsing-remitting forms of pro-inflammatory (e.g., autoimmune) diseases). In some embodiments, the agent administered to the subject (e.g., the fusion protein described herein) comprises one or more CD30L binding protein described herein, and the agent (e.g., a fusion protein described herein) is administered to the subject prior to, at the start of, at initial signs of, or during a flare. In some embodiments, the agent administered to the subject (e.g., the fusion protein described herein) comprises one or more CD30L binding protein described herein, the pro-inflammatory (e.g., autoimmune) disease is multiple sclerosis, rheumatoid arthritis, inflammatory bowel disease, systemic lupus erythematosus, myasthenia gravis, or psoriasis. In some embodiments, the agent administered to the subject (e.g., the fusion protein described herein) comprises one or more CD30L binding protein described herein, and the pro-inflammatory (e.g., autoimmune) disease is relapsing-remitting multiple sclerosis, relapsing-remitting rheumatoid arthritis, relapsing-remitting inflammatory bowel disease, relapsing-remitting systemic lupus erythematosus, relapsing-remitting myasthenia gravis, or relapsing-remitting psoriasis.

### 5.20 Kits

Provided herein are kits comprising an agent described herein, e.g., a fusion protein described herein, a nucleic acid molecule described herein (e.g., encoding a fusion protein described herein), a vector described herein e.g., comprising a nucleic acid molecule (e.g., described herein) encoding a fusion protein described herein), a conjugate described herein, a cell described herein, or a carrier described herein, a pharmaceutical composition described herein, a combination regimen described herein, or a combination composition described herein. In addition, the kit may comprise a liquid vehicle for solubilizing or diluting, and/or technical instructions. The technical instructions of the kit may contain information about administration and dosage and subject groups.

In some embodiments, the agent described herein, e.g., the fusion protein, the nucleic acid molecule, the vector described herein, the conjugate, the cell, the carrier, or the pharmaceutical composition is provided in a separate part of the kit, wherein the agent, e.g., the fusion protein, the nucleic acid molecule, the vector described herein, the conjugate, the cell, the carrier, the pharmaceutical composition, the combination regimen described herein, or the combination composition described herein is optionally lyophilized, spray-dried, or spray-freeze dried. The kit may further contain as a part a vehicle (e.g., buffer solution) for solubilizing the dried or lyophilized agent, e.g., the fusion protein, the nucleic acid molecule, the vector described herein, the conjugate, the cell, the carrier, the pharmaceutical composition, the combination regimen described herein, or the combination composition described herein.

In some embodiments, the kit comprises a single dose container. In some embodiments, the kit comprises a multi-dose container. In some embodiments, the kit comprises an administration device (e.g., an injector for intradermal injection or a syringe for intramuscular injection). In some embodiments, the kit comprises adjuvant in a separate container. The kit may further contain technical instructions for mixing the adjuvant prior to administration or for co-administration.

Any of the kits described herein may be used in any of the methods described herein (see, e.g., § 5.19).

### 6. EXAMPLES

TABLE OF CONTENTS

| | |
|---|---|
| 6.1 | Example 1. Immunomodulatory Fusion Protein Expression. |
| 6.2 | Example 2. Immunomodulatory Fusion Protein Mediated |
| | Dual Inhibition of IL-10 and TNFα Activity. |
| 6.3 | Example 3. Immunomodulatory Fusion Protein Mediated |
| | Dual Inhibition of TNFα and TL1A Activity. |
| 6.4 | Example 4. Immunomodulatory Fusion Protein Expression. |
| 6.5 | Example 5. IFP-13 and IFP-14 Mediated Inhibition of |
| | Integrin α4β7 MAdCAM-1 Binding. |
| 6.6 | Example 6. IFP-13 and IFP-14 Mediated Inhibition of |
| | TNFα Activity. |
| 6.7 | Example 7. IFP-13 and IFP-14 Mediated Inhibition of |
| | TL1A Activity. |
| 6.8 | Example 8. IFP-12 Mediated Inhibition of Integrin |
| | α4β7 MAdCAM-1 Binding. |
| 6.9 | Example 9. IFP-12 Mediated Inhibition of TNFα Activity. |
| 6.10 | Example 10. IFP-13, IFP-14, and IFP-16 Mediated |
| | Inhibition of Integrin α4β7 MAdCAM-1 Binding. |
| 6.11 | Example 11. IFP-13, IFP-14, and IFP-16 Mediated |
| | Inhibition of TNFα Activity. |
| 6.12 | Example 12. IFP-13, IFP-14, and IFP-16 Mediated |
| | Inhibition of TL1A Activity. |
| 6.1 | Example 1. Immunomodulatory Fusion Protein Expression. |

Immunoreceptor Fusion Proteins 1-10 (IFPs 1-10) (SEQ ID NOS: 593-612, respectively) (see Table 16) were generated using standard methods known in the art. Briefly, a DNA nucleic acid molecule encoding each fusion protein was synthesized and inserted into an expression plasmid. Expi293 cells (Thermo Fisher #A14527) were transfected using the Expi293 expression kit (Thermo Fisher #A14635) according to the manufacturer's protocol. Briefly, Expi293 cells were grown in suspension at 37° C., 8% CO2 in Expi293 growth medium (Thermo Fisher #A1435101). The cells were counted using a hemocytometer to ensure a density of 2.5-3 million cells per mL, and a viability above 95%, prior to transfection. Transfections were performed in 2.5 ml of cell containing medium (7.5-9 million cells per reaction). 1 μg/ml of plasmid DNA was pre-incubated with Opti-MEM for 5 minutes at room temperature (RT) and ExpiFectamine was pre-incubated with Opti-MEM for 5 minutes at RT. The plasmid mixture was subsequently mixed with the ExpiFectamine mixture and incubated for 10-20 minutes at RT. After incubation, the mixture was added to the Expi293 cells and incubated overnight. On day 1 post-transfection, ExpiFectamine Enhancer 1 and ExpiFectamine Enhancer 2 were added to the cell culture. On day 3 post-transfection, the supernatant was removed and maintained at −20° C., and the cells were discarded. The amino acid sequence of generated exemplary mature IFPs-1-10, is set forth in SEQ ID NOS: 593-612, respectively (see Table 16 herein).

The amino acid sequence of references proteins utilized in several of the following examples (and expressed generally as described above) is provided below in Table 17.

TABLE 17
Amino Acid Sequence of Exemplary Reference Proteins.

| | | SEQ |
|---|---|---|
| Description | Amio Acid Sequence | ID NO |
| Reference hIgG4 | AESKYGPPCPPCPAPEAAGGPSVFLFPPKPKDTLMISRTPEVTCVVVD | 628 |
| Fc fusion protein | VSQEDPEVQFNWYVDGVEVHNAKTKPREEQFNSTYRVVSVLTVLHQDW | |
| Variant Fc | LNGKEYKCKVSNKGLPSSIEKTISKAKGQPREPQVYTLPPSQEEMTKN | |
| (Fc) (without | QVSLTCLVKGFYPSDIAVEWESNGQPENNYKTTPPVLDSDGSFFLYSR | |
| signal sequence) | LTVDKSRWQEGNVFSCSVMHEALHNHYTQKSLSLSLG | |
| IL-10R Binder | AESKYGPPCPPCPAPEAAGGPSVFLFPPKPKDTLMISRTPEVTCVVVD | 629 |
| (hIL-10 Control) | VSQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTKN | |
| | QVSLTCLVKGFYPSDIAVEWESNGQPENNYKTTPPVLDSDGSFFLYSR | |
| | LTVDKSRWQEGNVFSCSVMHEALHNHYTQKSLSLSLGGGGGGGGSGGG | |
| | GSGGGGSSPGQGTQSENSCTHFPGNLPNMLRDLRDAFSRVKTFFQMKD | |
| | QLDNLLLKESLLEDFKGYLGCQALSEMIQFYLEEVMPQAENQDPDIKA | |
| | HVNSLGENLKTLRLRLRRCHRFLPCENKSKAVEQVKNAFNKLQEKGIY | |
| | KAMSEFDIFINYIEAYMTMKIRN | |
| TNFα Binder | AESKYGPPCPPCPAPEAAGGPSVFLFPPKPKDTLMISRTPEVTCVVVD | 627 |
| (TNFα Control) | VSQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTKN | |
| | QVSLTCLVKGFYPSDIAVEWESNGQPENNYKTTPPVLDSDGSFFLYSR | |
| | LTVDKSRWQEGNVFSCSVMHEALHNHYTQKSLSLSLGGGGGGGGSGGG | |
| | GSGGGGSDSKCGVSEYYNKEHDICCRLCPAGSYAEQLCTKDNDTVCNQ | |
| | CPPNTFLSIPNYISSCLSCRGKCINDHVEDKPCTATSNRICKCKENKT | |
| | CVLKTYDNSCRVCI | |
| IL-10R Binder | AESKYGPPCPPCPAPEAAGGPSVFLFPPKPKDTLMISRTPEVTCVVVD | 630 |
| (IL-10 Control) | VSQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTKN | |
| | QVSLTCLVKGFYPSDIAVEWESNGQPENNYKTTPPVLDSDGSFFLYSR | |
| | LTVDKSRWQEGNVFSCSVMHEALHNHYTQKSLSLSLGGGGGGGGSGGG | |
| | GSGGGGSKGRDSKPSPACDPMHGALAGIFKELRTTYRSVREALQTKDT | |
| | VYYVSLFHEQLLQEMLSPVGCRVTNELMQHYLDGVLPRAFHCGYDNAT | |
| | LNALHALSSSLSTLYQHMLKCPALACTGQTPAWTQFLDTEHKLDPWKG | |
| | TVKATAEMDLLLNYLETFLLQS | |
| TL1A Binder | AESKYGPPCPPCPAPEAAGGPSVFLFPPKPKDTLMISRTPEVTCVVVD | 631 |
| (TL1A Control) | VSQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTKN | |
| | QVSLTCLVKGFYPSDIAVEWESNGQPENNYKTTPPVLDSDGSFFLYSR | |
| | LTVDKSRWQEGNVFSCSVMHEALHNHYTQKSLSLSLGGGGGGGGSGGG | |
| | GSGGGGSSTYRVRSSGLTCSTCPPGTHKERDCSLNTETICKACGEGEY | |
| | TAHNNSLPKCLACKSCFNATEIETKSCDPTSDTICACREGYSINNLGE | |
| | CN | |

### 6.2 Example 2. Immunomodulatory Fusion Protein Mediated Dual Activation of IL-10 and Inhibition of TNFα Activity

The ability of IFP-1 (SEQ ID NO: 594) to activate IL-10/IL-10R signaling and inhibit the activity of TNFα was assessed.

IL-10 activity was assessed through assessment of the ability of IFP-1 to bind to the IL-10R using a cell-based reporter assay known in the art, namely the hIL-10 HEKBlue reporter cell line (InvivoGen #hkb-i110). Briefly, the hIL-10 HEKBlue reporter cell line expresses the hIL-10Rα and hIL-10Rβ subunits, human STAT3, and a STAT3-inducible SEAP (secreted embryonic alkaline phosphatase) reporter. Thereby, binding of IL-10 to the hIL-10R triggers JAK1/STAT3 signaling and the subsequent production of SEAP, which can be quantified using standard methods known in the art. The assay was conducted in accordance with the manufacturer's instructions. Briefly, the hIL-10 HEKBlue reporter cell line (InvivoGen #hkb-i110), was incubated with a dose titration of an IFP-1 (or indicated control (Fc (SEQ ID NO: 628), hIL-10 Control (SEQ ID NO: 629)) in DMEM medium with 10% heat-inactivated fetal bovine serum (FBS) at 37° C., 5% CO₂ for 20-24 hours. Subsequently, QuantiBlue substrate (InvivoGen #rep-qbs) was added to the samples and incubated for 1-3 hours. Colorimetric intensity was determined using a 96 well plate reader at 620-655 nm.

Likewise, TNFα activity was assessed using a commercially available HEK-Blue™ TNF-α cell line (InvivoGen; catalog number: hkb-tnfmyd). The cells are stably transfected with a SEAP reporter gene under the control of the IFN-β minimal promoter fused to five NF-κB (and five AP-1) binding sites and are unresponsive to IL-10 (MyD88 gene knockout). Stimulation of HEK-Blue™ TNF-α cells with TNF-α (through binding to TNFR1 and/or TNFR2) triggers the activation of the NF-κB-inducible promoter and the production of SEAP. The assay was conducted in accordance with the manufacturer's instructions. Briefly, HEK TNF-α cell lines (InvivoGen; catalog number: hkb-tnfmyd) were cultured and propagated for 3-4 passages according to manufacturer's instructions. TNFα (1 ng/mL) was incubated with each of IFP-1, or the indicated control (Fc (SEQ ID NO: 628), TNFα Control (SEQ ID NO: 627)) for 30 min at 37° C. before the addition of HEK TNF-α; and subsequently incubated for 20-24 hrs. The following day, QUANTI-Blue solution was added to the culture supernatants and SEAP levels determined at 620-655 nm as per manufacturer's instructions.

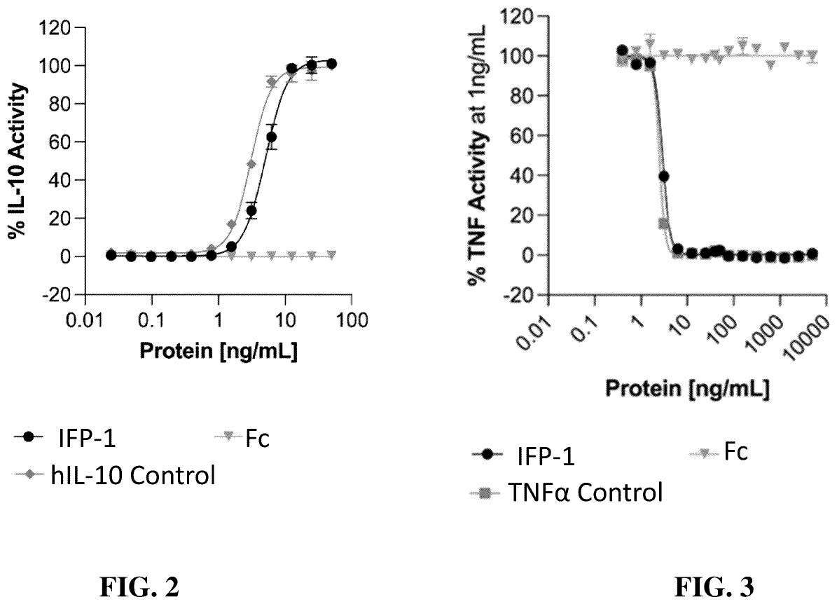

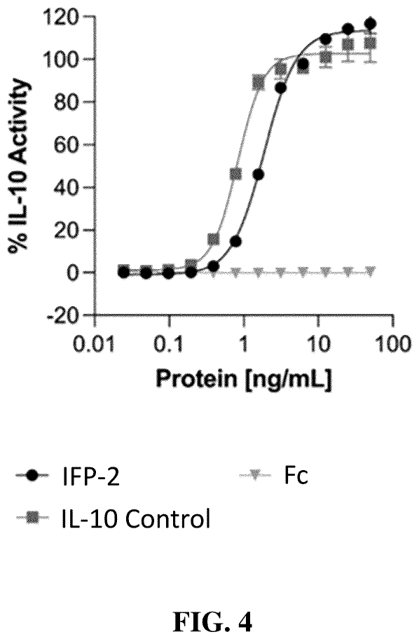

IFP-1 was shown to both bind the IL-10R with agonistic activity (FIG. 2) and neutralize TNFα (antagonistic activity) inhibiting TNFα mediated TNFR1/2 induced NFκB signaling (FIG. 3). IFP-2 (SEQ ID NO: 596) was shown to bind the IL-10R with agonistic activity (FIG. 4).

### 6.3 Example 3. Immunomodulatory Fusion Protein Mediated Dual Inhibition of TNFα and TL1A Activity

The ability of IFP-10 (SEQ ID NO: 612) to inhibit the activity of both TNFα and TL1A was assessed.

TNFα activity was assessed as described above in Example 2. TL1A activity was assessed using a commercially available TL1A responsive luciferase reporter Jurkat cell line. The cell line expresses luciferase under the control of NFκB response element and stably expresses human DR3. Activation of the NFκB signaling pathway by TL1A binding to DR3 induces luciferase expression. The assay was conducted in accordance with the manufacturer's instructions. Briefly, a serial dilution of IFP-10 or the indicated control (TNFα Control (SEQ ID NO: 627), TL1A Control (SEQ ID NO: 631)) was prepared and pre-incubated with 10 nM of human TL1A for one hour. Subsequently, each mixture was added to approximately 32,000 TL1A responsive luciferase reporter Jurkat cells per well in a white, clear-bottomed 96-well plate. The plate was then incubated at 37° C. and 5% CO₂ for about five hours. Afterward, 100 μL of OneStep™ Luciferase Detection Reagent (BPS Bioscience #60690) was added to each well. The plate was shaken at 400 RPM at room temperature for approximately 15 minutes before the luminescence was measured using a Varioskan.

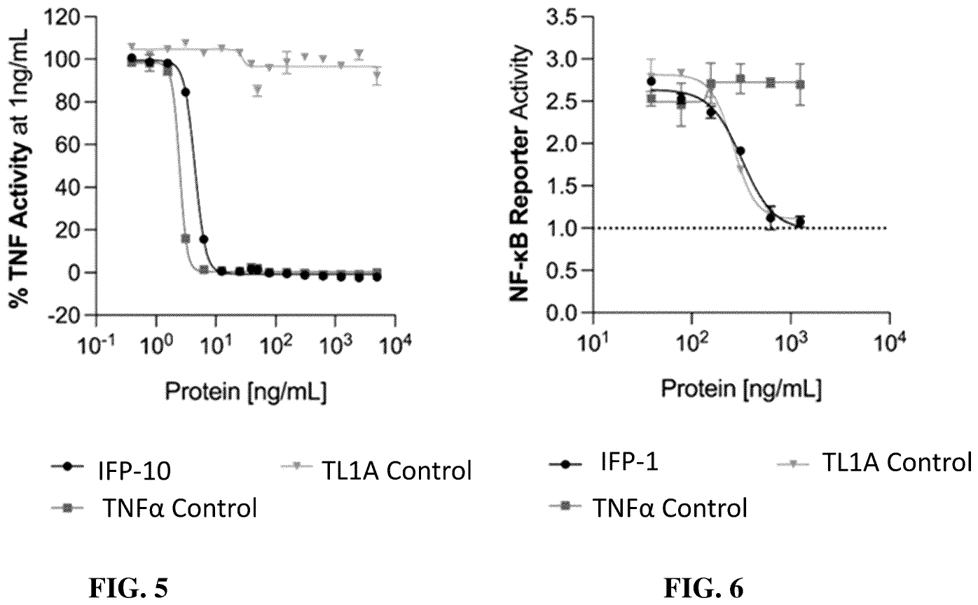

IFP-10 was shown to both neutralize TNFα (antagonistic activity) inhibiting TNFα mediated TNFR1/2 induced NFκB signaling (FIG. 5) and inhibit TL1A mediated NFκB activation (antagonistic activity) (FIG. 6).

### 6.4 Example 4. Immunomodulatory Fusion Protein Expression

Immunoreceptor Fusion Proteins 11-17 (IFPs 1-17) (SEQ ID NOS: 592, 613-626) (see Table 16 for pairings of polypeptides) were generated using standard methods known in the art. Briefly, a DNA nucleic acid molecule encoding each fusion protein was synthesized and inserted into an expression plasmid. Expi293 cells (Thermo Fisher #A14527) were transfected using the Expi293 expression kit (Thermo Fisher #A14635) according to the manufacturer's protocol. Briefly, Expi293 cells were grown in suspension at 37° C., 8% CO2 in Expi293 growth medium (Thermo Fisher #A1435101). The cells were counted using a hemocytometer to ensure a density of 2.5-3 million cells per mL, and a viability above 95%, prior to transfection. Transfections were performed in 2.5 ml of cell containing medium (7.5-9 million cells per reaction). 1 μg/ml of plasmid DNA was pre-incubated with Opti-MEM for 5 minutes at room temperature (RT) and ExpiFectamine was pre-incubated with Opti-MEM for 5 minutes at RT. The plasmid mixture was subsequently mixed with the ExpiFectamine mixture and incubated for 10-20 minutes at RT. After incubation, the mixture was added to the Expi293 cells and incubated overnight. On day 1 post-transfection, ExpiFectamine Enhancer 1 and ExpiFectamine Enhancer 2 were added to the cell culture. On day 3 post-transfection, the supernatant was removed and maintained at −20° C., and the cells were discarded. The amino acid sequence of generated exemplary mature IFPs-11-17, is set forth in) SEQ ID NOS: 592, 613-626 (see Table 16 for pairings of polypeptides).

The amino acid sequence of references proteins utilized in several of the following examples (and expressed generally as described above) is provided below in Table 18.

TABLE 18
Amino Acid Sequence of Exemplary Reference Proteins.

| | | | SEQ |
|---|---|---|---|
| Description | | Amio Acid Sequence | ID NO |
| Anti- | Heavy | QVQLVQSGAEVKKPGASVKVSCKGSGYTFTSYWMHWVRQ | 591 |
| Integrin Ab | Chain | APGQRLEWIGEIDPSESNTNYNQKFKGRVTLTVDISAST | |
| | | AYMELSSLRSEDTAVYYCARGGYDGWDYAIDYWGQGTLV | |
| | | TVSSASTKGPSVFPLAPSSKSTSGGTAALGCLVKDYFPE | |
| | | PVTVSWNSGALTSGVHTFPAVLQSSGLYSLSSVVTVPSS | |
| | | SLGTQTYICNVNHKPSNTKVDKKVEPKSCDKTHTCPPCP | |
| | | APELAGAPSVFLFPPKPKDTLMISRTPEVTCVVVDVSHE | |
| | | DPEVKFNWYVDGVEVHNAKTKPREEQYNSTYRVVSVLTV | |
| | | LHQDWLNGKEYKCKVSNKALPAPIEKTISKAKGQPREPQ | |
| | | VYTLPPSRDELTKNQVSLTCLVKGFYPSDIAVEWESNGQ | |
| | | PENNYKTTPPVLDSDGSFFLYSKLTVDKSRWQQGNVFSC | |
| | | SVMHEALHNHYTQKSLSLSPGK | |
| | Light | DVVMTQSPLSLPVTPGEPASISCRSSQSLAKSYGNTYLS | 592 |
| | Chain | WYLQKPGQSPQLLIYGISNRFSGVPDRFSGSGSGTDFTL | |
| | | KISRVEAEDVGVYYCLQGTHQPYTFGQGTKVEIKRTVAA | |
| | | PSVFIFPPSDEQLKSGTASVVCLLNNFYPREAKVQWKVD | |
| | | NALQSGNSQESVTEQDSKDSTYSLSSTLTLSKADYEKHK | |
| | | VYACEVTHQGLSSPVTKSFNRGEC | |

| | | |
|---|---|---|
| TNFα Binder | AESKYGPPCPPCPAPEAAGGPSVFLFPPKPKDTLMISRT | 627 |
| (TNFα Control) | PEVTCVVVDVSQEDPEVQFNWYVDGVEVHNAKTKPREEQ | |
| | FNSTYRVVSVLTVLHQDWLNGKEYKCKVSNKGLPSSIEK | |
| | TISKAKGQPREPQVYTLPPSQEEMTKNQVSLTCLVKGFY | |
| | PSDIAVEWESNGQPENNYKTTPPVLDSDGSFFLYSRLTV | |
| | DKSRWQEGNVFSCSVMHEALHNHYTQKSLSLSLGGGGGG | |
| | GGSGGGGSGGGGSDSKCGVSEYYNKEHDICCRLCPAGSY | |
| | AEQLCTKDNDTVCNQCPPNTFLSIPNYISSCLSCRGKCI | |
| | NDHVEDKPCTATSNRICKCKENKTCVLKTYDNSCRVCI | |

### 6.5 Example 5. IFP-13 and IFP-14 Mediated Inhibition of Integrin α4β7 MAdCAM-1 Binding

The ability of IFP-13 (SEQ ID NOS: 592 and 618) and IFP-14 (SEQ ID NOS: 592 and 620) to inhibit binding of the integrin α4β7 to MAdCAM-1 was assessed.

Briefly, a commercially available integrin α4β7: MAdCAM-1 [Biotinylated] Inhibitor Screening ELISA Kit (Catalog #EP-155-96 tests, Acro Biosystems) was utilized according to manufacturer's instructions. Briefly, a plate was coated with human integrin α4β7, the respective agent added (IFP-13, IFP-14, or anti-integrin antibody control (SEQ ID NOS: 591-592)), biotinylated human MAdCAM-1 added, and binding assessed using Streptavidin-HRP with a colorimetric substrate to determine the IC50 of each agent by comparing OD readings.

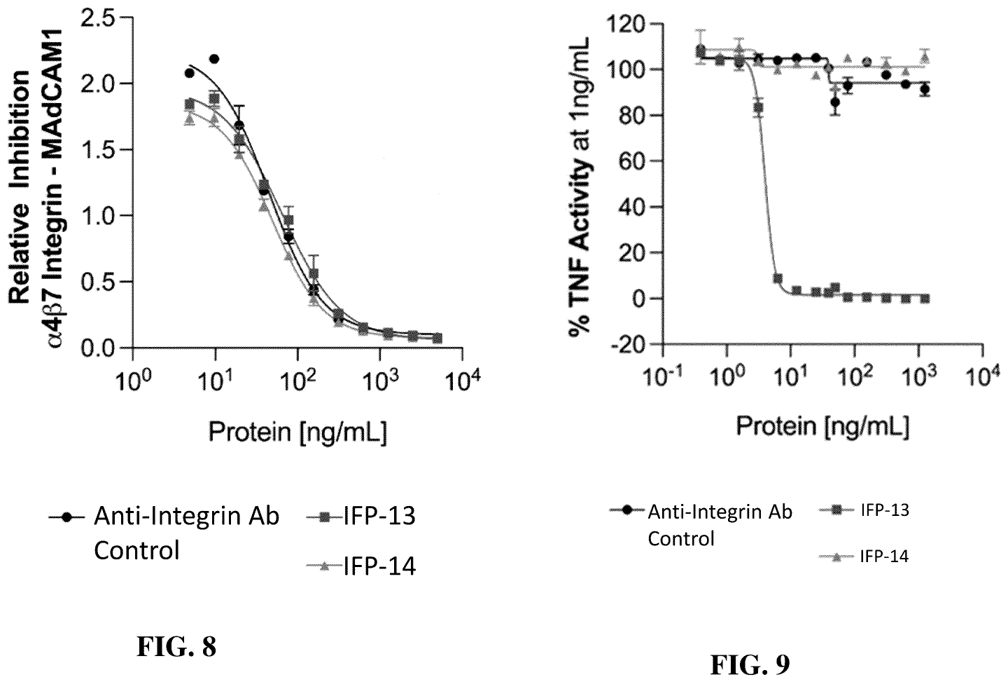

IFP-13 and IFP-14 both showed a dose dependent inhibition of integrin α4β7 MAdCAM-1 binding (FIG. 8) (along with positive control).

### 6.6 Example 6. IFP-13 and IFP-14 Mediated Inhibition of TNFα Activity

The ability of IFP-13 (SEQ ID NOS: 592 and 618) and IFP-14 (SEQ ID NOS: 592 and 620) to inhibit TNFα activity was assessed.

Briefly, TNFα activity was assessed using a commercially available HEK-Blue™ TNF-α cell line (InvivoGen; catalog number: hkb-tnfmyd). The cells are stably transfected with a SEAP reporter gene under the control of the IFN-β minimal promoter fused to five NF-κB (and five AP-1) binding sites and are unresponsive to IL-10 (MyD88 gene knockout). Stimulation of HEK-Blue™ TNF-α cells with TNF-α (through binding to TNFR1 and/or TNFR2) triggers the activation of the NF-κB-inducible promoter and the production of SEAP. The assay was conducted in accordance with the manufacturer's instructions. Briefly, HEK TNF-α cell lines (InvivoGen; catalog number: hkb-tnfmyd) were cultured and propagated for 3-4 passages according to manufacturer's instructions. TNFα (1 ng/mL) was incubated with each of IFP-13, IFP-14, or anti-integrin antibody control (SEQ ID NOS: 591-592) for 30 min at 37° C. before the addition of HEK TNF-α; and subsequently incubated for 20-24 hrs. The following day, QUANTI-Blue solution was added to the culture supernatants and SEAP levels determined at 620-655 nm as per manufacturer's instructions.

IFP-13 showed a dose dependent inhibition of TNFα mediated TNFR1/2 induced NFκB signaling (FIG. 9) (along with positive control); and IFP-14 did not show dose dependent inhibition of TNFα mediated TNFR1/2 induced NFκB signaling (FIG. 9).

### 6.7 Example 7. IFP-13 and IFP-14 Mediated Inhibition of TL1A Activity

The ability of IFP-13 (SEQ ID NOS: 592 and 618) and IFP-14 (SEQ ID NOS: 592 and 620) to inhibit TL1A activity was assessed.

Briefly, TL1A activity was assessed using a commercially available TL1A responsive luciferase reporter Jurkat cell line. The cell line expresses luciferase under the control of NFκB response element and stably expresses human DR3. Activation of the NFκB signaling pathway by TL1A binding to DR3 induces luciferase expression. The assay was conducted in accordance with the manufacturer's instructions. Briefly, a serial dilution of IFP-13, IFP-14, or anti-integrin antibody control (SEQ ID NOS: 591-592) was prepared and pre-incubated with 10 nM of human TL1A for one hour. Subsequently, each mixture was added to approximately 32,000 TL1A responsive luciferase reporter Jurkat cells per well in a white, clear-bottomed 96-well plate. The plate was then incubated at 37° C. and 5% CO₂ for about five hours. Afterward, 100 μL of OneStep™ Luciferase Detection Reagent (BPS Bioscience #60690) was added to each well. The plate was shaken at 400 RPM at room temperature for approximately 15 minutes before the luminescence was measured using a Varioskan.

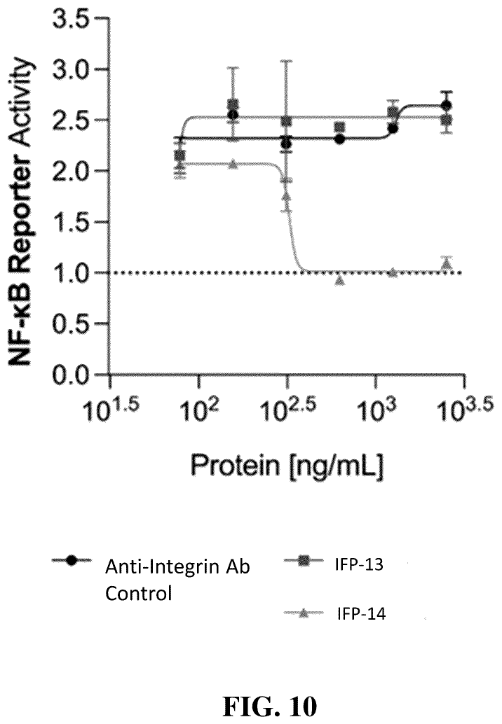

IFP-14 showed a dose dependent inhibition of TL1A mediated NFκB activation (antagonistic activity) (FIG. 10) (along with positive control); and IFP-13 did not show dose dependent inhibition of TL1A mediated NFκB activation (antagonistic activity) (FIG. 10).

### 6.8 Example 8. IFP-12 Mediated Inhibition of Integrin α4β7 MAdCAM-1 Binding

The ability of IFP-12 (SEQ ID NO: 616) to inhibit binding of the integrin α4β7 to MAdCAM-1 was assessed.

Briefly, a commercially available integrin α4β7: MAdCAM-1 [Biotinylated] Inhibitor Screening ELISA Kit (Catalog #EP-155-96 tests, Acro Biosystems) was utilized according to manufacturer's instructions. Briefly, a plate was coated with human integrin α4β7, the respective agent added (IFP-12 or negative control), biotinylated human MAdCAM-1 added, and binding assessed using Streptavidin-HRP with a colorimetric substrate to determine the IC50 of each agent by comparing OD readings.

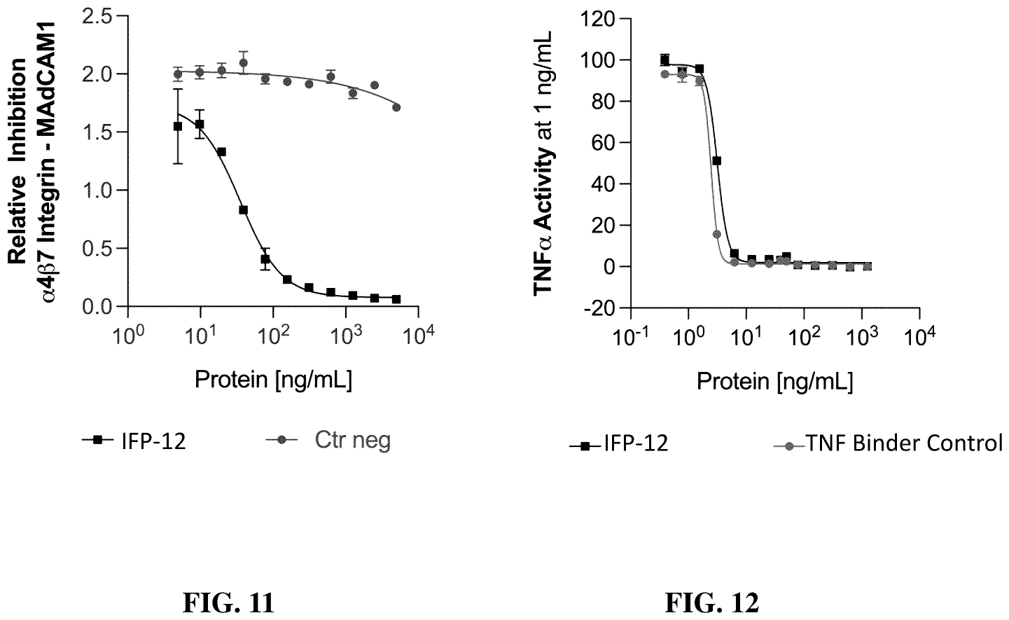

IFP-12 showed a dose dependent inhibition of integrin α4β7 MAdCAM-1 binding (FIG. 11).

### 6.9 Example 9. IFP-12 Mediated Inhibition of TNFα Activity

The ability of IFP-12 (SEQ ID NO: 616) to inhibit TNFα activity was assessed.

Briefly, TNFα activity was assessed using a commercially available HEK-Blue™ TNF-α cell line (InvivoGen; catalog number: hkb-tnfmyd). The cells are stably transfected with a SEAP reporter gene under the control of the IFN-β minimal promoter fused to five NF-κB (and five AP-1) binding sites and are unresponsive to IL-10 (MyD88 gene knockout). Stimulation of HEK-Blue™ TNF-α cells with TNF-α (through binding to TNFR1 and/or TNFR2) triggers the activation of the NF-κB-inducible promoter and the production of SEAP. The assay was conducted in accordance with the manufacturer's instructions. Briefly, HEK TNF-α cell lines (InvivoGen; catalog number: hkb-tnfmyd) were cultured and propagated for 3-4 passages according to manufacturer's instructions. TNFα (1 ng/mL) was incubated with each of IFP-12 or TNF binder control (SEQ ID NO: 627) for 30 min at 37° C. before the addition of HEK TNF-α; and subsequently incubated for 20-24 hrs. The following day, QUANTI-Blue solution was added to the culture supernatants and SEAP levels determined at 620-655 nm as per manufacturer's instructions.

IFP-12 showed a dose dependent inhibition of TNFα mediated TNFR1/2 induced NFκB signaling (FIG. 12).

### 6.10 Example 10. IFP-3, IFP-4, and IFP-6 Mediated Inhibition of Integrin α4β7 MAdCAM-1 Binding

The ability of IFP-13 (SEQ ID NOS: 592 and 518), IFP-14 (SEQ ID NOS: 592 and 620), and IFP-16 (SEQ ID NOS: 592 and 624) to inhibit binding of the integrin α4β7 to MAdCAM-1 was assessed.

Briefly, a commercially available integrin α4β7: MAdCAM-1 [Biotinylated] Inhibitor Screening ELISA Kit (Catalog #EP-155-96 tests, Acro Biosystems) was utilized according to manufacturer's instructions. Briefly, a plate was coated with human integrin α4β7, the respective agent added (IFP-13, IFP-14, IFP-16, or anti-integrin antibody control (SEQ ID NOS: 591-592)), biotinylated human MAdCAM-1 added, and binding assessed using Streptavidin-HRP with a colorimetric substrate to determine the IC50 of each agent by comparing OD readings.

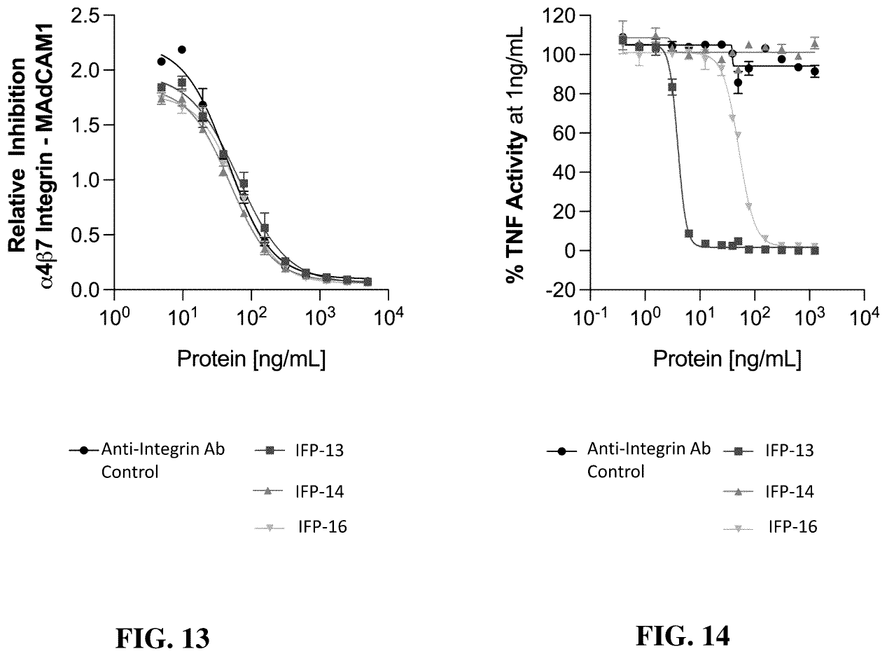

IFP-13, IFP-14, and IFP-16 each showed a dose dependent inhibition of integrin α4β7 MAdCAM-1 binding (FIG. 13).

### 6.11 Example 11. IFP-13, IFP-14, and IFP-16 Mediated Inhibition of TNFα Activity

The ability of IFP-13 (SEQ ID NOS: 592 and 618), IFP-14 (SEQ ID NOS: 592 and 620), and IFP-16 (SEQ ID NOS: 592 and 624) to inhibit TNFα activity was assessed.

Briefly, TNFα activity was assessed using a commercially available HEK-Blue™ TNF-α cell line (InvivoGen; catalog number: hkb-tnfmyd). The cells are stably transfected with a SEAP reporter gene under the control of the IFN-β minimal promoter fused to five NF-κB (and five AP-1) binding sites and are unresponsive to IL-10 (MyD88 gene knockout). Stimulation of HEK-Blue™ TNF-α cells with TNF-α (through binding to TNFR1 and/or TNFR2) triggers the activation of the NF-κB-inducible promoter and the production of SEAP. The assay was conducted in accordance with the manufacturer's instructions. Briefly, HEK TNF-α cell lines (InvivoGen; catalog number: hkb-tnfmyd) were cultured and propagated for 3-4 passages according to manufacturer's instructions. TNFα (1 ng/mL) was incubated with each of IFP-13, IFP-14, IFP-16, or anti-integrin antibody control (SEQ ID NOS: 591-592) for 30 min at 37° C. before the addition of HEK TNF-α; and subsequently incubated for 20-24 hrs. The following day, QUANTI-Blue solution was added to the culture supernatants and SEAP levels determined at 620-655 nm as per manufacturer's instructions.

IFP-13 and IFP-16 each showed a dose dependent inhibition of TNFα mediated TNFR1/2 induced NFκB signaling (FIG. 14) (along with positive control); and IFP-14 did not show dose dependent inhibition of TNFα mediated TNFR1/2 induced NFκB signaling (FIG. 14).

### 6.12 Example 12. IFP-13, IFP-14, and IFP-16 Mediated Inhibition of TL1A Activity

The ability of IFP-13 (SEQ ID NOS: 592 and 618), IFP-14 (SEQ ID NOS: 592 and 620), and IFP-16 (SEQ ID NOS: 592 and 624) to inhibit TL1A activity was assessed.

Briefly, TL1A activity was assessed using a commercially available TL1A responsive luciferase reporter Jurkat cell line. The cell line expresses luciferase under the control of NFκB response element and stably expresses human DR3. Activation of the NFκB signaling pathway by TL1A binding to DR3 induces luciferase expression. The assay was conducted in accordance with the manufacturer's instructions. Briefly, a serial dilution of IFP-13, IFP-14, IFP-16, or anti-integrin antibody control (SEQ ID NOS: 591-592) was prepared and pre-incubated with 10 nM of human TL1A for one hour. Subsequently, each mixture was added to approximately 32,000 TL1A responsive luciferase reporter Jurkat cells per well in a white, clear-bottomed 96-well plate. The plate was then incubated at 37° C. and 5% CO₂ for about five hours. Afterward, 100 μL of OneStep™ Luciferase Detection Reagent (BPS Bioscience #60690) was added to each well. The plate was shaken at 400 RPM at room temperature for approximately 15 minutes before the luminescence was measured using a Varioskan.

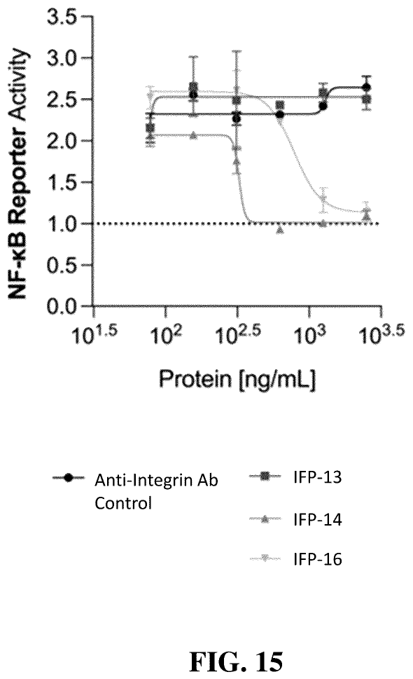

IFP-14 and IFP-16 showed a dose dependent inhibition of TL1A mediated NFκB activation (antagonistic activity) (FIG. 15) (along with positive control); and IFP-13 did not show dose dependent inhibition of TL1A mediated NFκB activation (antagonistic activity) (FIG. 15).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1. A fusion protein comprising any two or more of
(a) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386; and any one or more of
(b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454;
(c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464;
(d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576;
(a-1) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386;
(b-1) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454;
(c-1) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and/or
(d-1) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

2.-119. (canceled)

120. A fusion protein comprising
(a) an integrin binding domain; and one or more of
(b) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 34-386;
(c) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 387-454;
(d) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 455-464; and/or
(e) a protein comprising an amino acid sequence at least about 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 465-576.

121.-258. (canceled)

259. A conjugate comprising the fusion protein of claim 1 operably connected to a heterologous moiety.

260. A radioligand comprising the fusion protein of claim 1 operably connected to a radionuclide.

261. A fusion protein comprising the fusion protein of claim 1 operably connected to a heterologous protein.

262. One or more nucleic acid molecule encoding the fusion protein (or one or more polypeptide thereof) of claim 1.

263. A vector comprising the nucleic acid molecule of claim 262.

264. A carrier comprising the fusion protein of claim 1.

265.-266. (canceled)

267. A cell comprising the fusion protein of claim 1.

268. (canceled)

269. A pharmaceutical composition comprising the fusion protein of claim 1 and a pharmaceutically acceptable excipient.

270. A combination regimen comprising at least two proteins (or one or more nucleic acid molecule encoding the same), wherein each of the at least two proteins comprises an IL-10 binding protein described herein, a TL1A binding protein described herein, a TNFα binding protein described herein, a CD30L binding protein described herein, or a fusion protein described herein.

271. A combination composition comprising at least two proteins (or one or more nucleic acid molecule encoding the same), wherein each of the at least two proteins comprises an IL-10 binding protein described herein, a TL1A binding protein described herein, a TNFα binding protein described herein, a CD30L binding protein described herein, or a fusion protein described herein.

272. A kit comprising the fusion protein of claim 1; and
optionally instructions for using any one or more of the foregoing.

273. A method of delivering a fusion protein to a subject, the method comprising administering to the subject the fusion protein of claim 1 to the subject.

274. A method of inhibiting or reducing binding of a plurality of respective receptor ligand interactions in a subject in need thereof, the method comprising administering to the subject the fusion protein of claim 1, to thereby inhibit or reduce binding of a plurality of respective receptor ligand interactions in the subject.

275. A method of inhibiting or reducing signaling mediated by a plurality of respective receptor ligand interactions in a subject in need thereof, the method comprising administering to the subject the fusion protein of claim 1, to thereby inhibit or reduce signaling mediated by a plurality of respective receptor ligand interactions in in the subject.

276. A method of suppressing or reducing a pro-inflammatory immune response in a subject in need thereof, the method comprising administering to the subject the fusion protein of claim 1, to thereby suppress or reduce a pro-inflammatory immune response in the subject.

277. A method of preventing, treating, or ameliorating a disease in a subject in need thereof, the method comprising administering to the subject the fusion protein of claim 1 or ameliorate the disease in the subject.

278.-281. (canceled)

282. A conjugate comprising the fusion protein of claim 120 operably connected to a heterologous moiety.

283. A radioligand comprising the fusion protein of claim 120 operably connected to a radionuclide.

284. A fusion protein comprising the fusion protein of claim 120 operably connected to a heterologous protein.

285. One or more nucleic acid molecule encoding the fusion protein (or one or more polypeptide thereof) of claim 120.

286. A vector comprising the nucleic acid molecule of claim 285.

287. A carrier comprising the fusion protein of claim 120.

288. A cell comprising the fusion protein of claim 120.

289. A pharmaceutical composition comprising the fusion protein of claim 120; and a pharmaceutically acceptable excipient.

290. A kit comprising the fusion protein of claim 120; and optionally instructions for using any one or more of the foregoing.

291. A method of delivering a fusion protein to a subject, the method comprising administering to the subject the fusion protein of claim 120 to the subject.

292. A method of inhibiting or reducing binding of a plurality of respective receptor ligand interactions in a subject in need thereof, the method comprising administering to the subject the fusion protein of claim 120 binding of a plurality of respective receptor ligand interactions in the subject.

293. A method of inhibiting or reducing signaling mediated by a plurality of respective receptor ligand interactions in a subject in need thereof, the method comprising administering to the subject the fusion protein of claim 120, to thereby inhibit or reduce signaling mediated by a plurality of respective receptor ligand interactions in in the subject.

294. A method of suppressing or reducing a pro-inflammatory immune response in a subject in need thereof, the method comprising administering to the subject the fusion protein of claim 120, to thereby suppress or reduce a pro-inflammatory immune response in the subject.

295. A method of preventing, treating, or ameliorating a disease in a subject in need thereof, the method comprising administering to the subject the fusion protein of claim 120, to thereby prevent, treat, or ameliorate the disease in the subject.
